(19) United States
(12) Patent Application Publication
(10) Pub. No.: US 2024/0115692 A1
(43) Pub. Date: Apr. 11, 2024

(54) COMPOSITIONS AND METHODS FOR INDUCING IMMUNE RESPONSES

(71) Applicant: Arcturus Therapeutics, Inc., San Diego, CA (US)

(72) Inventors: Sean Michael SULLIVAN, Escondido, CA (US); Daiki MATSUDA, San Diego, CA (US); Kiyoshi TACHIKAWA, San Diego, CA (US); Padmanabh CHIVUKULA, San Diego, CA (US); Priya Prakash KARMALI, San Diego, CA (US); Jared Henry DAVIS, Poway, CA (US); Yanjie BAO, San Diego, CA (US)

(21) Appl. No.: 18/351,392

(22) Filed: Jul. 12, 2023

Related U.S. Application Data
(63) Continuation of application No. 17/196,890, filed on Mar. 9, 2021, now Pat. No. 11,759,515.
(60) Provisional application No. 63/073,900, filed on Sep. 2, 2020.
(60) Provisional application No. 62/987,191, filed on Mar. 9, 2020.

(51) Int. Cl. A61K 39/215; A61K 9/51; A61K 39/12; A61K 47/10; A61K 47/20; A61K 47/26; C07K 14/005; C07K 14/18; C12N 7/00; C12N 15/86

(52) U.S. Cl. CPC A61K 39/215; A61K 9/5123; A61K 39/12; A61K 47/10; A61K 47/20; A61K 47/26; C07K 14/005; C07K 14/1808; C12N 7/00; C12N 15/86; A61K 2039/53

(57) ABSTRACT

Provided herein are nucleic acid molecules encoding viral replication proteins and antigenic proteins or fragments thereof. Also provided herein are compositions that include nucleic acid molecules encoding viral replication and antigenic proteins, and lipids. Nucleic acid molecules provided herein are useful for inducing immune responses.

### CROSS-REFERENCES TO RELATED APPLICATIONS

This application claims the benefit of U.S. Provisional Application No. 62/987,191, filed Mar. 9, 2020 and U.S. Provisional Application No. 63/073,900, filed Sep. 2, 2020.

### REFERENCE TO A SEQUENCE LISTING

The instant application contains a Sequence Listing which has been submitted electronically in ASCII format and is hereby incorporated by reference in its entirety. Said ASCII copy, created on Mar. 8, 2021 is named 049386-530001US_SequenceListing_ST25.txt and is 390,698 bytes in size.

### TECHNICAL FIELD

The present disclosure relates generally to inducing immune responses against infectious agents and tumor antigens and more specifically to self-transcribing and replicating RNA for antigen expression.

### BACKGROUND

Infectious diseases and cancer represent significant burdens on health worldwide. According to the World Health Organization (WHO), lower respiratory tract infection was the deadliest infectious disease worldwide in 2016, causing approximately 3 million deaths. Current control measures to curb the rapid worldwide spread of infection diseases, such as national lockdowns, closure of work places and schools, and reduction of international travel are threatening to result in a global economic recession to an extent not seen since the Great Depression.

Cancer is the second leading cause of death globally, accounting for approximately 9.6 million deaths worldwide in 2018. Cancer is a large group of diseases that can affect almost any organ or tissue in the body. Cancer burden continues to grow globally, exerting physical, emotional, and financial strains on patients and health care providers. Self-replicating ribonucleic acids (RNAs), e.g., derived from viral replicons, are useful for expression of proteins, such as heterologous proteins, for a variety of purposes, such as expression of therapeutic proteins and expression of antigens for vaccines. A desirable property of such replicons is the ability for sustained expression of the protein.

Few treatments for infections caused by viruses and eukaryotic organisms are available, and resistance to antibiotics for the treatment of bacterial infections is increasing. In addition, rapid responses, including rapid vaccine development, are required to effectively control emerging infectious diseases and pandemics. Moreover, many cancer treatments include costly and painful surgeries and chemotherapies that are often unsuccessful or only modestly prolong life despite serious side effects. Thus, there exists a need for the prevention and/or treatment of infectious diseases and cancer.

### SUMMARY

In one aspect, the present disclosure provides a nucleic acid molecule comprising a first polynucleotide encoding one or more viral replication proteins, wherein the first polynucleotide is codon-optimized as compared to a wild-type polynucleotide encoding the one or more viral replication proteins; and a second polynucleotide comprising a first transgene encoding a first antigenic protein or a fragment thereof.

In some embodiments, the one or more viral replication proteins may be alphavirus proteins or rubivirus proteins.

In some embodiments, the alphavirus proteins are from Venezuelan Equine Encephalitis Virus (VEEV), Eastern Equine Encephalitis Virus (EEEV), Everglades Virus (EVEV), Mucambo Virus (MUCV), Semliki Forest Virus (SFV), Pixuna Virus (PIXV), Middleburg Virus (MIDV), Chikungunya Virus (CHIKV), O'Nyong-Nyong Virus (ONNV), Ross River Virus (RRV), Barmah Forest Virus (BFV), Getah Virus (GETV), Sagiyama Virus (SAGV), Bebaru Virus (BEBV), Mayaro Virus (MAYV), Una Virus (UNAV), Sindbis Virus (SINV), Aura Virus (AURAV), Whataroa Virus (WHAV), Babanki Virus (BABV), Kyzylagach Virus (KYZV), Western Equine Encephalitis Virus (WEEV), Highland J Virus (HJV), Fort Morgan Virus (FMV), Ndumu Virus (NDUV), Salmonid Alphavirus (SAV), Buggy Creek Virus (BCRV), or any combination thereof.

In some embodiments, the first polynucleotide encodes a polyprotein comprising an alphavirus nsP1 protein, an alphavirus nsP2 protein, an alphavirus nsP3 protein, an alphavirus nsP4 protein, or any combination thereof.

In some embodiments, the first polynucleotide encodes a polyprotein comprising an alphavirus nsP1 protein, an alphavirus nsP2 protein, an alphavirus nsP3 protein, or any combination thereof, and an alphavirus nsP4 protein.

In some embodiments, the nucleic acid molecule further comprises a first intergenic region between a sequence encoding the polyprotein comprising an alphavirus nsP1 protein, an alphavirus nsP2 protein, an alphavirus nsP3 protein, or any combination thereof, and a sequence encoding an alphavirus nsP4 protein.

In some embodiments, the first intergenic region comprises an alphavirus sequence.

In some embodiments, the first polynucleotide comprises a sequence having at least 80% identity to a sequence of SEQ ID NO:72.

In some embodiments, the nucleic acid molecule further comprises a 5′ untranslated region (UTR), such as a viral 5′ UTR, a non-viral 5′ UTR, or a combination of viral and non-viral 5′ UTR sequences. In some embodiments, the 5′ UTR comprises an alphavirus 5′ UTR.

In some embodiments, the alphavirus 5′ UTR comprises a Venezuelan Equine Encephalitis Virus (VEEV), Eastern Equine Encephalitis Virus (EEEV), Everglades Virus (EVEV), Mucambo Virus (MUCV), Semliki Forest Virus (SFV), Pixuna Virus (PIXV), Middleburg Virus (MIDV), Chikungunya Virus (CHIKV), O'Nyong-Nyong Virus (ONNV), Ross River Virus (RRV), Barmah Forest Virus (BFV), Getah Virus (GETV), Sagiyama Virus (SAGV), Bebaru Virus (BEBV), Mayaro Virus (MAYV), Una Virus (UNAV), Sindbis Virus (SINV), Aura Virus (AURAV), Whataroa Virus (WHAV), Babanki Virus (BABV), Kyzylagach Virus (KYZV), Western Equine Encephalitis Virus (WEEV), Highland J Virus (HJV), Fort Morgan Virus (FMV), Ndumu Virus (NDUV), Salmonid Alphavirus (SAV), or Buggy Creek Virus (BCRV) 5′ UTR sequence.

In some embodiments, the 5′ UTR comprises a sequence of SEQ ID NO:73, SEQ ID NO:74, or SEQ ID NO:75.

In some embodiments, the nucleic acid molecule further comprises a 3′ untranslated region (UTR). In some embodiments, the 3′ UTR comprises a viral 3′ UTR, a non-viral 3′ UTR, or a combination of viral and non-viral 3′ UTR sequences. In some embodiments, the 3′ UTR comprises an alphavirus 3′ UTR.

In some embodiments, the alphavirus 3′ UTR comprises a Venezuelan Equine Encephalitis Virus (VEEV), Eastern Equine Encephalitis Virus (EEEV), Everglades Virus (EVEV), Mucambo Virus (MUCV), Semliki Forest Virus (SFV), Pixuna Virus (PIXV), Middleburg Virus (MIDV), Chikungunya Virus (CHIKV), O'Nyong-Nyong Virus (ONNV), Ross River Virus (RRV), Barmah Forest Virus (BFV), Getah Virus (GETV), Sagiyama Virus (SAGV), Bebaru Virus (BEBV), Mayaro Virus (MAYV), Una Virus (UNAV), Sindbis Virus (SINV), Aura Virus (AURAV), Whataroa Virus (WHAV), Babanki Virus (BABV), Kyzylagach Virus (KYZV), Western Equine Encephalitis Virus (WEEV), Highland J Virus (HJV), Fort Morgan Virus (FMV), Ndumu Virus (NDUV), Salmonid Alphavirus (SAV), or Buggy Creek Virus (BCRV) 3′ UTR sequence.

In some embodiments, the 3′ UTR comprises a poly-A sequence. In some embodiments, the 3′ UTR comprises a sequence of SEQ ID NO:76.

In some embodiments, the antigenic protein is a viral protein, a bacterial protein, a fungal protein, a protozoan protein, a parasite protein, or a tumor protein.

In some embodiments, the viral protein is an orthomyxovirus protein, a paramyxovirus protein, a picornavirus protein, a flavivirus protein, a filovirus protein, a rhabdovirus protein, a togavirus protein, an arterivirus protein, a bunyavirus protein, an arenavirus protein, a reovirus protein, a bornavirus protein, a retrovirus protein, an adenovirus protein, a herpesvirus protein, a polyomavirus protein, a papillomavirus protein, a poxvirus protein, or a hepadnavirus protein.

In some embodiments, the antigenic protein is an influenza virus protein, a respiratory syncytial virus (RSV) protein, a human immunodeficiency virus (HIV) protein, a hepatitis C virus (HCV) protein, a cytomegalovirus (CMV) protein, a Lassa Fever Virus (LFV) protein, an Ebola Virus (EBOV) protein, a Mycobacterium protein, a Bacillus protein, a Yersinia protein, a Streptococcus protein, a Pseudomonas protein, a Shigella protein, a Campylobacter protein, a Salmonella protein, a Plasmodium protein, or a Toxoplasma protein.

In some embodiments, the tumor protein is a kidney cancer, renal cancer, urinary bladder cancer, prostate cancer, uterine cancer, breast cancer, cervical cancer, ovarian cancer, lung cancer, liver cancer, stomach cancer, colon cancer, rectal cancer, oral cavity cancer, pharynx cancer, pancreatic cancer, thyroid cancer, melanoma, skin cancer, head and neck cancer, brain cancer, hematopoietic cancer, leukemia, lymphoma, bone cancer, or sarcoma protein.

In some embodiments, the second polynucleotide comprises at least two transgenes.

In some embodiments, a second transgene encodes a second antigenic protein or a fragment thereof or an immunomodulatory protein.

In some embodiments, the second polynucleotide further comprises a sequence encoding a 2A peptide, an internal ribosomal entry site (IRES), or a combination thereof, located between transgenes.

In some embodiments, the immunomodulatory protein is a cytokine, a chemokine, or an interleukin.

In some embodiments, the first and second transgenes encode viral proteins, bacterial proteins, fungal proteins, protozoan proteins, parasite proteins, tumor proteins, immunomodulatory proteins, or any combination thereof.

In some embodiments, the first polynucleotide is located 5′ of the second polynucleotide.

In some embodiments, the nucleic acid molecule further comprises a second intergenic region located between the first polynucleotide and the second polynucleotide.

In some embodiments, the second intergenic region comprises a sequence having at least 85% identity to a sequence of SEQ ID NO:77.

In some embodiments, the nucleic acid molecule is a DNA molecule; or an RNA molecule, wherein T is substituted with U.

In some embodiments, the DNA molecule further comprises a promoter. In some embodiments, the promoter is located 5′ of the 5′UTR.

In some embodiments, the promoter is a T7 promoter, a T3 promoter, or an SP6 promoter.

In some embodiments, the RNA molecule is a self-replicating RNA molecule.

In some embodiments, the RNA molecule further comprises a 5′ cap. In some embodiments, the 5′ cap has a Cap 1 structure, a Cap 1 (m6A) structure, a Cap 2 structure, a Cap 0 structure, or any combination thereof.

In another aspect, provided herein is a nucleic acid molecule comprising a sequence of SEQ ID NO:78; or a sequence of SEQ ID NO:72, SEQ ID NO:73, SEQ ID NO:76, and SEQ ID NO:77, wherein T is substituted with U.

In some embodiments, the nucleic acid molecule is an RNA molecule.

In some embodiments, the nucleic acid molecule further comprises a 5′ cap having a Cap 1 structure.

In yet another aspect, provided herein is a nucleic acid molecule comprising a first polynucleotide comprising a sequence having at least 80% identity to a sequence of SEQ ID NO:72; and a second polynucleotide comprising a first transgene encoding a first antigenic protein or a fragment thereof.

In some embodiments, the nucleic acid molecule further comprises a 5′ untranslated region (UTR).

In some embodiments, the 5′ UTR comprises a viral 5′ UTR, a non-viral 5′ UTR, or a combination of viral and non-viral 5′ UTR sequences. In some embodiments, the 5′ UTR comprises an alphavirus 5′ UTR.

In some embodiments, the alphavirus 5′ UTR comprises a Venezuelan Equine Encephalitis Virus (VEEV), Eastern Equine Encephalitis Virus (EEEV), Everglades Virus (EVEV), Mucambo Virus (MUCV), Semliki Forest Virus (SFV), Pixuna Virus (PIXV), Middleburg Virus (MIDV), Chikungunya Virus (CHIKV), O'Nyong-Nyong Virus (ONNV), Ross River Virus (RRV), Barmah Forest Virus (BFV), Getah Virus (GETV), Sagiyama Virus (SAGV), Bebaru Virus (BEBV), Mayaro Virus (MAYV), Una Virus (UNAV), Sindbis Virus (SINV), Aura Virus (AURAV), Whataroa Virus (WHAV), Babanki Virus (BABV), Kyzylagach Virus (KYZV), Western Equine Encephalitis Virus (WEEV), Highland J Virus (HIV), Fort Morgan Virus (FMV), Ndumu Virus (NDUV), Salmonid Alphavirus (SAV), or Buggy Creek Virus (BCRV) 5′ UTR sequence.

In some embodiments, the 5′ UTR comprises a sequence of SEQ ID NO:73, SEQ ID NO:74, or SEQ ID NO:75.

In some embodiments, the nucleic acid molecule further comprises a 3′ untranslated region (UTR).

In some embodiments, the 3′ UTR comprises a viral 3′ UTR, a non-viral 3′ UTR, or a combination of viral and non-viral 3′ UTR sequences. In some embodiments, the 3′ UTR comprises an alphavirus 3′ UTR.

In some embodiments, the alphavirus 3′ UTR comprises a Venezuelan Equine Encephalitis Virus (VEEV), Eastern Equine Encephalitis Virus (EEEV), Everglades Virus (EVEV), Mucambo Virus (MUCV), Semliki Forest Virus (SFV), Pixuna Virus (PIXV), Middleburg Virus (MIDV), Chikungunya Virus (CHIKV), O'Nyong-Nyong Virus (ONNV), Ross River Virus (RRV), Barmah Forest Virus (BFV), Getah Virus (GETV), Sagiyama Virus (SAGV), Bebaru Virus (BEBV), Mayaro Virus (MAYV), Una Virus (UNAV), Sindbis Virus (SINV), Aura Virus (AURAV), Whataroa Virus (WHAV), Babanki Virus (BABV), Kyzylagach Virus (KYZV), Western Equine Encephalitis Virus (WEEV), Highland J Virus (HJV), Fort Morgan Virus (FMV), Ndumu Virus (NDUV), Salmonid Alphavirus (SAV), or Buggy Creek Virus (BCRV) 3′ UTR sequence.

In some embodiments, the 3′ UTR comprises a poly-A sequence.

In some embodiments, the 3′ UTR comprises a sequence of SEQ ID NO:76.

In some embodiments, the antigenic protein is a viral protein, a bacterial protein, a fungal protein, a protozoan protein, a parasite protein, or a tumor protein.

In some embodiments, the viral protein is an orthomyxovirus protein, a paramyxovirus protein, a picornavirus protein, a flavivirus protein, a filovirus protein, a rhabdovirus protein, a togavirus protein, an arterivirus protein, a bunyavirus protein, an arenavirus protein, a reovirus protein, a bornavirus protein, a retrovirus protein, an adenovirus protein, a herpesvirus protein, a polyomavirus protein, a papillomavirus protein, a poxvirus protein, or a hepadnavirus protein.

In some embodiments, the antigenic protein is an influenza virus protein, a respiratory syncytial virus (RSV) protein, a human immunodeficiency virus (HIV) protein, a hepatitis C virus (HCV) protein, a cytomegalovirus (CMV) protein, a Lassa Fever Virus (LFV) protein, an Ebola Virus (EBOV) protein, a Mycobacterium protein, a Bacillus protein, a Yersinia protein, a Streptococcus protein, a Pseudomonas protein, a Shigella protein, a Campylobacter protein, a Salmonella protein, a Plasmodium protein, or a Toxoplasma protein.

In some embodiments, the tumor protein is a kidney cancer, renal cancer, urinary bladder cancer, prostate cancer, uterine cancer, breast cancer, cervical cancer, ovarian cancer, lung cancer, liver cancer, stomach cancer, colon cancer, rectal cancer, oral cavity cancer, pharynx cancer, pancreatic cancer, thyroid cancer, melanoma, skin cancer, head and neck cancer, brain cancer, hematopoietic cancer, leukemia, lymphoma, bone cancer, or sarcoma protein.

In some embodiments, the second polynucleotide comprises at least two transgenes. In some embodiments, a second transgene encodes a second antigenic protein or a fragment thereof or an immunomodulatory protein.

In some embodiments, the second polynucleotide further comprises a sequence encoding a 2A peptide, an internal ribosomal entry site (IRES), or a combination thereof, located between transgenes.

In some embodiments, the immunomodulatory protein is a cytokine, a chemokine, or an interleukin.

In some embodiments, the first and second transgenes encode viral proteins, bacterial proteins, fungal proteins, protozoan proteins, parasite proteins, tumor proteins, immunomodulatory proteins, or any combination thereof.

In some embodiments, the first polynucleotide is located 5′ of the second polynucleotide.

In some embodiments, the nucleic acid molecule further comprises a second intergenic region located between the first polynucleotide and the second polynucleotide. In some embodiments, the second intergenic region comprises a sequence having at least 85% identity to a sequence of SEQ ID NO:77.

In some embodiments, the nucleic acid molecule is a DNA molecule; or an RNA molecule, wherein T is substituted with U.

In some embodiments, the DNA molecule further comprises a promoter. In some embodiments, the promoter is located 5′ of the 5′UTR.

In some embodiments, the promoter is a T7 promoter, a T3 promoter, or an SP6 promoter.

In some embodiments, the RNA molecule is a self-replicating RNA molecule.

In some embodiments, the RNA molecule further comprises a 5′ cap. In some embodiments, the 5′ cap has a Cap 1 structure, a Cap 1 (m6A) structure, a Cap 2 structure, a Cap 0 structure, or any combination thereof.

In yet another aspect, provided herein is a composition comprising any one of the nucleic acid molecules described herein.

In some embodiments, the lipid comprises an ionizable cationic lipid. In some embodiments, the ionizable cationic lipid has a structure of

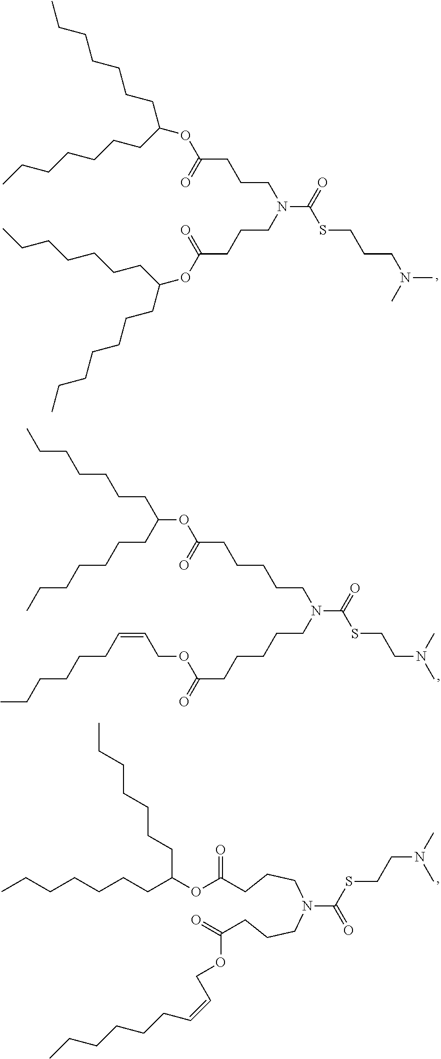

or a pharmaceutically acceptable salt thereof.

In yet another aspect, provided herein is a pharmaceutical composition comprising any one of the nucleic acid molecules described herein, and a lipid formulation.

In some embodiments, the lipid formulation comprises an ionizable cationic lipid. In some embodiments, the ionizable cationic lipid has a structure of

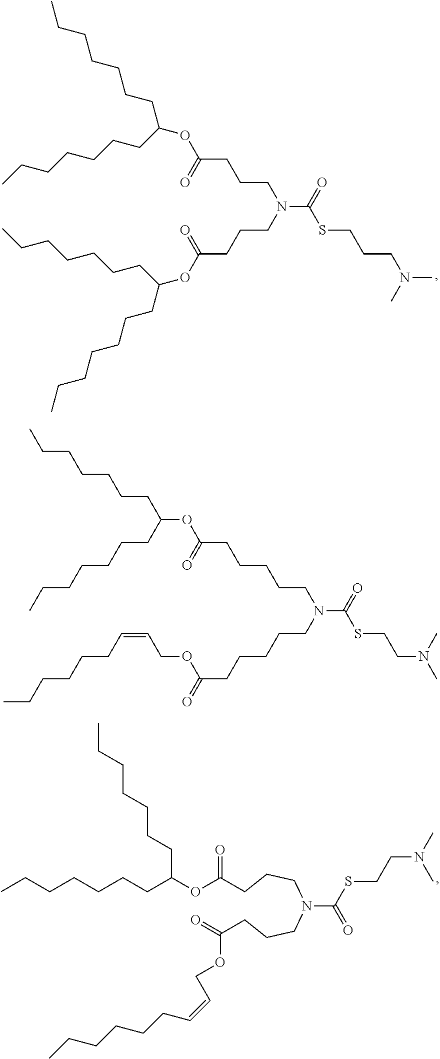

or a pharmaceutically acceptable salt thereof.

In yet another aspect, provided herein is a method of inducing an immune response in a subject comprising administering to the subject an effective amount of any one of the nucleic acid molecules described herein.

In some embodiments, the method comprises administering the nucleic acid molecule intramuscularly, subcutaneously, intradermally, transdermally, intranasally, orally, sublingually, intravenously, intraperitoneally, topically, by aerosol, or by a pulmonary route.

In yet another aspect, provided herein is a method of inducing an immune response in a subject comprising administering to the subject an effective amount of any one of the compositions described herein.

In some embodiments, the method comprises administering the composition intramuscularly, subcutaneously, intradermally, transdermally, intranasally, orally, sublingually, intravenously, intraperitoneally, topically, by aerosol, or by a pulmonary route.

In yet another aspect, provided herein is a method of inducing an immune response in a subject comprising administering to the subject an effective amount of any one of the pharmaceutical compositions described herein.

In some embodiments, the method may comprise administering the pharmaceutical composition intramuscularly, subcutaneously, intradermally, transdermally, intranasally, orally, sublingually, intravenously, intraperitoneally, topically, by aerosol, or by a pulmonary route.

In yet another aspect, the present disclosure provides any of the nucleic acid molecules described herein for use in inducing an immune response to the first antigenic protein or fragment thereof.

In yet another aspect, the present disclosure provides use of any one of the nucleic acid molecules described herein in the manufacture of a medicament for inducing an immune response to the first antigenic protein or fragment thereof.

### BRIEF DESCRIPTION OF THE DRAWINGS

FIG. 1 shows a schematic illustrating one aspect of STARR™ technology.

FIGS. 2A-2D show characterization of STARR™ technology with firefly luciferase transgene expression. (2A) Firefly luciferase (FLuc) expression from STARR™ Fluc, SINV FLuc, and mRNA FLuc was monitored up to day 28 by In Vivo Imaging System (IVIS). The average of total flux (p/s) from 6 injection sites in a mouse group was plotted at each time point with a standard error of mean, SEM. (2B) IVIS picture of three mice (6 injection sites) per group on day 14 is shown for each group that was administered with the test article labeled below the picture. (2C) Luciferase expression from mice that were intramuscularly injected with STARR™ FLuc was monitored by IVIS up to 63 days post administration. (2D) Effect of prior administration of replicon backbone was examined for STARR™ (upper panel) and SINV (lower panel). Replicon encoding FLuc was IM injected at 7 days post dose of replicon with homologous backbone with an irrelevant gene/sequence (labeled STARR™ irr or SINV irr) at day 0. As a reference, a mouse group with PBS administration at day 0 was included in each of STARR™ and SINV group.

FIG. 3 shows that STARR™ elicits antigen-specific IFN-gamma response. Enzyme-linked immune absorbent spot ELISpot was used to count the number of splenocytes that were specifically stimulated by an antigen peptide of the same amino acid sequence encoded in TA STARR™. Neither no peptide (cell only) nor irrelevant peptide (Bgal) elicited significant IFN-gamma from splenocytes from mice vaccinated with STARR™ FLuc or TA STARR™. Stimulation with AH1-A5 peptide resulted in the detection of IFN-gamma-producing cells specifically from the mice that were vaccinated with TA STARR™. Concanavalin A (ConA) was used as a positive control of IFN-gamma production.

FIGS. 4A-4F illustrate reduced tumor growth rate by TA STARR™ vaccination in a CT26 syngeneic mouse model. CT26 murine colorectal carcinoma cells (5×10⁵) were subcutaneously implanted in 10-week old female BALB/c mice (n=8 per group). On days 1 and 8, the mice were vaccinated with STARR™ FLuc, a negative control, or TA STARR™, which encodes AH1A5 epitope. Tumor growth was monitored in mice vaccinated with (4A) STARR™ FLuc without checkpoint inhibitor treatment; (4B) STARR™ FLuc with a combination anti-PD1/PDL1 treatment; (4C) STARR™ FLuc with a combination anti-CTLA4 treatment; (4D) STARR™ vaccine without checkpoint inhibitor treatment; (4E) STARR™ vaccine with a combination treatment of anti-PD1 and anti-PDL1; and (4F) STARR™ vaccine with a combination treatment of anti-CTLA4. The individual tumor growth curves from a mouse group that were administered with STARR™ FLuc and TA STARR™ are shown in upper and lower panels, respectively.

FIG. 5 illustrates prolonged protection by combination treatment of TA STARR™ Vaccine with checkpoint inhibitors. Mice that were treated with TA STARR™ combined with anti-PD1/PDL1 or anti-CTLA4 were found to be resistant to tumor development following the CT26 challenge at day 25 to 42. Naïve mice were used as a control for the CT26 tumor growth.

FIGS. 6A-6C show results from AH1-tetramer staining of CD8+ T-cells in the form of (6A) a graph and (6B and 6C) plots. Splenocytes from the mice group with combination treatment of TA STARR™ and anti-PD1/PDL1 at day 42 were stained with AH1 (H-2Ld)-tetramer. The staining was specific to CD8+ T cells from the mouse group with TA STARR™ treatment, and the population represented 9-17% of total CD8+ T cells from the splenocytes.

FIG. 7 shows HAI titers obtained for self-replicating RNA (STARR™) and mRNA constructs encoding the hemagglutinin of influenza virus A/California/07/2009 (H1N1).

FIGS. 8A-8B show (8A) RNA replication levels and (8B) luciferase reporter gene expression levels for the indicated self-replicating (replicon) RNAs as compared to mRNA.

FIGS. 9A-9C shows duration of luciferase reporter gene expression for self-replicating (replicon) RNA (STARR™), such as (9A) STARR™ FLuc, (9B) STARR™ FLuc IRES-E3L, and (9C) STARR™ FLuc IRES E3L (short 3′ UTR) as compared to mRNA.

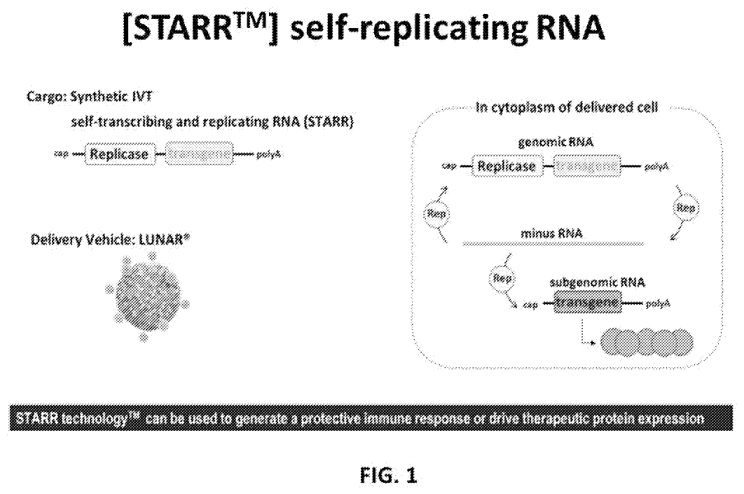

### DETAILED DESCRIPTION

The present disclosure relates to self-replicating RNAs and nucleic acids encoding the same for expression of transgenes such as antigenic proteins and tumor antigens, for example. Also provided herein are methods of administration (e.g., to a host, such as a mammalian subject) of self-replicating RNAs, whereby the self-replicating RNA is translated in vivo and the heterologous protein-coding sequence is expressed and, e.g., can elicit an immune response to the heterologous protein-coding sequence in the recipient or provide a therapeutic effect, where the heterologous protein-coding sequence is a therapeutic protein. Self-replicating RNAs provided herein are useful as vaccines that can be rapidly generated and that can be effective at low and/or single doses. The present disclosure further relates to methods of inducing an immune response using self-replicating RNAs provided herein.

### Definitions

As used herein, the singular forms “a,” “an,” and “the” include plural references unless the context clearly dictates otherwise. Thus, for example, references to “the method” includes one or more methods, and/or steps of the type described herein which will become apparent to those persons skilled in the art upon reading this disclosure and so forth.

“About” as used herein when referring to a measurable value such as an amount, a temporal duration, and the like, is meant to encompass variations of +20%, or ±10%, or ±5%, or even ±1% from the specified value, as such variations are appropriate for the disclosed methods or to perform the disclosed methods.

As used herein, the term “fragment,” when referring to a protein or nucleic acid, for example, means any shorter sequence than the full-length protein or nucleic acid. Accordingly, any sequence of a nucleic acid or protein other than the full-length nucleic acid or protein sequence can be a fragment. In some aspects, a protein fragment includes an epitope. In other aspects, a protein fragment is an epitope.

As used herein, the term “nucleic acid” refers to any deoxyribonucleic acid (DNA) molecule, ribonucleic acid (RNA) molecule, or nucleic acid analogues. A DNA or RNA molecule can be double-stranded or single-stranded and can be of any size. Exemplary nucleic acids include, but are not limited to, chromosomal DNA, plasmid DNA, cDNA, cell-free DNA (cfDNA), mitochondrial DNA, chloroplast DNA, viral DNA, mRNA, tRNA, rRNA, long non-coding RNA, siRNA, micro RNA (miRNA or miR), hnRNA, and viral RNA. Exemplary nucleic analogues include peptide nucleic acid, morpholino- and locked nucleic acid, glycol nucleic acid, and threose nucleic acid. As used herein, the term “nucleic acid molecule” is meant to include fragments of nucleic acid molecules as well as any full-length or non-fragmented nucleic acid molecule, for example. As used herein, the terms “nucleic acid” and “nucleic acid molecule” can be used interchangeably, unless context clearly indicates otherwise.

As used herein, the term “protein” refers to any polymeric chain of amino acids. The terms “peptide” and “polypeptide” can be used interchangeably with the term protein, unless context clearly indicates otherwise, and can also refer to a polymeric chain of amino acids. The term “protein” encompasses native or artificial proteins, protein fragments and polypeptide analogs of a protein sequence. A protein may be monomeric or polymeric. The term “protein” encompasses fragments and variants (including fragments of variants) thereof, unless otherwise contradicted by context.

In general, “sequence identity” or “sequence homology,” which can be used interchangeably, refer to an exact nucleotide-to-nucleotide or amino acid-to-amino acid correspondence of two polynucleotides or polypeptide sequences, respectively. Typically, techniques for determining sequence identity include determining the nucleotide sequence of a polynucleotide and/or determining the amino acid sequence encoded thereby or the amino acid sequence of a polypeptide, and comparing these sequences to a second nucleotide or amino acid sequence.

As used herein, the term “percent (%) sequence identity” or “percent (%) identity,” also including “homology,” refers to the percentage of amino acid residues or nucleotides in a sequence that are identical with the amino acid residues or nucleotides in a reference sequence after aligning the sequences and introducing gaps, if necessary, to achieve the maximum percent sequence identity, and not considering any conservative substitutions as part of the sequence identity. Thus, two or more sequences (polynucleotide or amino acid) can be compared by determining their “percent identity,” also referred to as “percent homology.” The percent identity to a reference sequence (e.g., nucleic acid or amino acid sequences), which may be a sequence within a longer molecule (e.g., polynucleotide or polypeptide), may be calculated as the number of exact matches between two optimally aligned sequences divided by the length of the reference sequence and multiplied by 100. Percent identity may also be determined, for example, by comparing sequence information using the advanced BLAST computer program, including version 2.2.9, available from the National Institutes of Health. The BLAST program is based on the alignment method of Karlin and Altschul, Proc. Natl. Acad. Sci. USA 87:2264-2268 (1990) and as discussed in Altschul et al., J. Mol. Biol. 215:403-410 (1990); Karlin and Altschul, Proc. Natl. Acad. sci. USA 90:5873-5877 (1993); and Altschul et al., Nucleic Acids Res. 25:3389-3402 (1997). Briefly, the BLAST program defines identity as the number of identical aligned symbols (i.e., nucleotides or amino acids), divided by the total number of symbols in the shorter of the two sequences. The program may be used to determine percent identity over the entire length of the sequences being compared. Default parameters are provided to optimize searches with short query sequences, for example, with the blastp program. The program also allows use of an SEG filter to mask-off segments of the query sequences as determined by the SEG program of Wootton and Federhen, Computers and Chemistry 17: 149-163 (1993). Ranges of desired degrees of sequence identity are approximately 80% to 100% and integer values in between. Percent identities between a reference sequence and a claimed sequence can be at least 80%, at least 85%, at least 90%, at least 95%, at least 98%, at least 99%, at least 99.5%, or at least 99.9%. In general, an exact match indicates 100% identity over the length of the reference sequence. Additional programs and methods for comparing sequences and/or assessing sequence identity include the Needleman-Wunsch algorithm (see, e.g., the EMBOSS Needle aligner available at ebi.ac.uk/Tools/psa/emboss needle/, optionally with default settings), the Smith-Waterman algorithm (see, e.g., the EMBOSS Water aligner available at ebi.ac.uk/Tools/psa/emboss water/, optionally with default settings), the similarity search method of Pearson and Lipman, 1988, Proc. Natl. Acad. Sci. USA 85, 2444, or computer programs which use these algorithms (GAP, BESTFIT, FASTA, BLAST P, BLAST N and TFASTA in Wisconsin Genetics Software Package, Genetics Computer Group. 575 Science Drive, Madison, Wis.). In some aspects, reference to percent sequence identity refers to sequence identity as measured using BLAST (Basic Local Alignment Search Tool). In other aspects, ClustalW is used for multiple sequence alignment. Optimal alignment may be assessed using any suitable parameters of a chosen algorithm, including default parameters.

The term “expression” refers to the process by which a nucleic acid sequence or a polynucleotide is transcribed from a DNA template (such as into mRNA or other RNA transcript) and/or the process by which a transcribed mRNA or other RNA is subsequently translated into peptides, polypeptides, or proteins. Transcripts and encoded polypeptides may be collectively referred to as “gene product.”

As used herein, “operably linked,” “operable linkage,” “operatively linked,” or grammatical equivalents thereof refer to juxtaposition of genetic elements, e.g., a promoter, an enhancer, a polyadenylation sequence, etc., wherein the elements are in a relationship permitting them to operate in the expected manner. For instance, a regulatory element, which can comprise promoter and/or enhancer sequences, is operatively linked to a coding region if the regulatory element helps initiate transcription of the coding sequence. There may be intervening residues between the regulatory element and coding region so long as this functional relationship is maintained.

As used herein, the term “drug” or “medicament,” means a pharmaceutical formulation or composition as described herein.

The phrases “administered in combination” or “combined administration” means that two or more agents are administered to a subject at the same time or within an interval such that there may be an overlap of an effect of each agent on the patient. In some embodiments, they are administered within about 60, 30, 15, 10, 5, or 1 minute of one another. In some embodiments, the administrations of the agents are spaced sufficiently closely together such that a combinatorial (e.g., a synergistic) effect is achieved.

As used herein, the terms “self-replicating RNA,” “self-transcribing and self-replicating RNA,” “self-amplifying RNA (saRNA),” and “replicon” may be used interchangeably, unless context clearly indicates otherwise. Generally, the term “replicon” or “viral replicon” refers to a self-replicating subgenomic RNA derived from a viral genome that includes viral genes encoding non-structural proteins important for viral replication and that lacks viral genes encoding structural proteins. A self-replicating RNA can encode further subgenomic RNAs that are not able to self-replicate.

Nucleic Acid Molecules

In some embodiments, provided herein are nucleic acid molecules comprising: (i) a first polynucleotide encoding one or more viral replication proteins, wherein the first polynucleotide is codon-optimized as compared to a wild-type polynucleotide encoding the one or more viral replication proteins; and (ii) a second polynucleotide comprising a first transgene encoding a first antigenic protein or a fragment thereof.

An RNA molecule can encode a single polypeptide immunogen or multiple polypeptides. Multiple immunogens can be presented as a single polypeptide immunogen (fusion polypeptide) or as separate polypeptides. If immunogens are expressed as separate polypeptides from a replicon then one or more of these may be provided with an upstream IRES or an additional viral promoter element. Alternatively, multiple immunogens may be expressed from a polyprotein that encodes individual immunogens fused to a short autocatalytic protease (e.g. foot-and-mouth disease virus 2A protein), or as inteins.

Also provided herein, in some embodiments, are nucleic acid molecules comprising: (i) a first polynucleotide comprising a sequence having at least 80% identity to a sequence of SEQ ID NO:72; and (ii) a second polynucleotide comprising a first transgene encoding a first antigenic protein or a fragment thereof.

Codon Optimization

In some embodiments, first polynucleotides of nucleic acid molecules provided herein encoding one or more viral replication proteins include codon-optimized sequences. As used herein, the term “codon-optimized” means a polynucleotide, nucleic acid sequence, or coding sequence has been redesigned as compared to a wild-type or reference polynucleotide, nucleic acid sequence, or coding sequence by choosing different codons without altering the amino acid sequence of the encoded protein. Accordingly, codon-optimization generally refers to replacement of codons with synonymous codons to optimize expression of a protein while keeping the amino acid sequence of the translated protein the same. Codon optimization of a sequence can increase protein expression levels (Gustafsson et al., Codon bias and heterologous protein expression. 2004, Trends Biotechnol 22: 346-53) of the encoded proteins, for example, and provide other advantages. Variables such as codon usage preference as measured by codon adaptation index (CAI), for example, the presence or frequency of U and other nucleotides, mRNA secondary structures, cis-regulatory sequences, GC content, and other variables may correlate with protein expression levels (Villalobos et al., Gene Designer: a synthetic biology tool for constructing artificial DNA segments. 2006, BMC Bioinformatics 7:285).

Any method of codon optimization can be used to codon optimize polynucleotides and nucleic acid molecules provided herein, and any variable can be altered by codon optimization. Accordingly, any combination of codon optimization methods can be used. Exemplary methods include the high codon adaptation index (CAI) method, the Low U method, and others. The CAI method chooses a most frequently used synonymous codon for an entire protein coding sequence. As an example, the most frequently used codon for each amino acid can be deduced from 74,218 protein-coding genes from a human genome. The Low U method targets U-containing codons that can be replaced with a synonymous codon with fewer U moieties, generally without changing other codons. If there is more than one choice for replacement, the more frequently used codon can be selected. Any polynucleotide, nucleic acid sequence, or codon sequence provided herein can be codon-optimized. This method may be used in conjunction with the disclosed RNAs to design coding sequences that are to be synthesized with, for example, 5-methoxyuridine or N1-methyl pseudouridine. Methods of codon optimization in combination with the use of a modified nucleotide monomer are described in U.S. 2018/0327471, the contents of which are herein incorporated by reference.

In some embodiments, the nucleotide sequence of any region of the RNA or DNA templates described herein may be codon optimized. Preferably, the primary cDNA template may include reducing the occurrence or frequency of appearance of certain nucleotides in the template strand. For example, the occurrence of a nucleotide in a template may be reduced to a level below 25% of said nucleotides in the template. In further examples, the occurrence of a nucleotide in a template may be reduced to a level below 20% of said nucleotides in the template. In some examples, the occurrence of a nucleotide in a template may be reduced to a level below 16% of said nucleotides in the template. Preferably, the occurrence of a nucleotide in a template may be reduced to a level below 15%, and preferably may be reduced to a level below 12% of said nucleotides in the template.

In some embodiments, the nucleotide reduced is uridine. For example, the present disclosure provides nucleic acids with altered uracil content wherein at least one codon in the wild-type sequence has been replaced with an alternative codon to generate a uracil-altered sequence. Altered uracil sequences can have at least one of the following properties:
(i) an increase or decrease in global uracil content (i.e., the percentage of uracil of the total nucleotide content in the nucleic acid of a section of the nucleic acid, e.g., the open reading frame);
(ii) an increase or decrease in local uracil content (i.e., changes in uracil content are limited to specific subsequences);
(iii) a change in uracil distribution without a change in the global uracil content;
(iv) a change in uracil clustering (e.g., number of clusters, location of clusters, or distance between clusters); or
(v) combinations thereof.

In some embodiments, the percentage of uracil nucleobases in the nucleic acid sequence is reduced with respect to the percentage of uracil nucleobases in the wild-type nucleic acid sequence. For example, 30% of nucleobases may be uracil in the wild-type sequence but the nucleobases that are uracil are preferably lower than 15%, preferably lower than 12% and preferably lower than 10% of the nucleobases in the nucleic acid sequences of the disclosure. The percentage uracil content can be determined by dividing the number of uracil in a sequence by the total number of nucleotides and multiplying by 100.

In some embodiments, the percentage of uracil nucleobases in a subsequence of the nucleic acid sequence is reduced with respect to the percentage of uracil nucleobases in the corresponding subsequence of the wild-type sequence. For example, the wild-type sequence may have a 5′-end region (e.g., 30 codons) with a local uracil content of 30%, and the uracil content in that same region could be reduced to preferably 15% or lower, preferably 12% or lower and preferably 10% or lower in the nucleic acid sequences of the disclosure. These subsequences can also be part of the wild-type sequences of the heterologous 5′ and 3′ UTR sequences of the present disclosure.

In some embodiments, codons in the nucleic acid sequence of the disclosure reduce or modify, for example, the number, size, location, or distribution of uracil clusters that could have deleterious effects on protein translation. Although lower uracil content is desirable in certain aspects, the uracil content, and in particular the local uracil content, of some subsequences of the wild-type sequence can be greater than the wild-type sequence and still maintain beneficial features (e.g., increased expression).

In some embodiments, the uracil-modified sequence induces a lower Toll-Like Receptor (TLR) response when compared to the wild-type sequence. Several TLRs recognize and respond to nucleic acids. Double-stranded (ds)RNA, a frequent viral constituent, has been shown to activate TLR3. Single-stranded (ss)RNA activates TLR7. RNA oligonucleotides, for example RNA with phosphorothioate internucleotide linkages, are ligands of human TLR8. DNA containing unmethylated CpG motifs, characteristic of bacterial and viral DNA, activate TLR9.

As used herein, the term “TLR response” is defined as the recognition of single-stranded RNA by a TLR7 receptor, and preferably encompasses the degradation of the RNA and/or physiological responses caused by the recognition of the single-stranded RNA by the receptor. Methods to determine and quantify the binding of an RNA to a TLR7 are known in the art. Similarly, methods to determine whether an RNA has triggered a TLR7-mediated physiological response (e.g., cytokine secretion) are well known in the art. In some embodiments, a TLR response can be mediated by TLR3, TLR8, or TLR9 instead of TLR7. Suppression of TLR7-mediated response can be accomplished via nucleoside modification. RNA undergoes over a hundred different nucleoside modifications in nature. Human rRNA, for example, has ten times more pseudouracil (′P) and 25 times more 2′-O-methylated nucleosides than bacterial rRNA. Bacterial RNA contains no nucleoside modifications, whereas mammalian RNAs have modified nucleosides such as 5-methylcytidine (m5C), N6-methyladenosine (m6A), inosine and many 2′-O-methylated nucleosides in addition to N7-methylguanosine (m7G).

In some embodiments, the uracil content of polynucleotides disclosed herein is less than about 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2% or 1% of the total nucleobases in the sequence in the reference sequence. In some embodiments, the uracil content of polynucleotides disclosed herein is between about 5% and about 25%. In some embodiments, the uracil content of polynucleotides disclosed herein is between about 15% and about 25%.

In some embodiments, first polynucleotides of nucleic acid molecules provided herein comprise a sequence having at least 80%, at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9%, and any number or range in between, identity to a sequence of SEQ ID NO:72. In some embodiments, first polynucleotides of nucleic acid molecules provided herein comprise a sequence of SEQ ID NO:72.

In some aspects, first polynucleotides and second polynucleotides of nucleic acid molecules provided herein are included in the same (i.e., a single) or in separate nucleic acid molecules. Generally, first polynucleotides and second polynucleotides of nucleic acid molecules provided herein are included in a single nucleic acid molecule. In one aspect, the first polynucleotide is located 5′ of the second polynucleotide. In one aspect, first polynucleotides and second polynucleotides of nucleic acid molecules provided herein are included in separate nucleic acid molecules. In yet another aspect, first polynucleotides and second polynucleotides are included in two separate nucleic acid molecules.

In some aspects, first polynucleotides and second polynucleotides are included in the same (i.e., a single) nucleic acid molecule. First polynucleotides and second polynucleotides of nucleic acid molecules provided herein can be contiguous, i.e., adjacent to each other without nucleotides in between. In one aspect, an intergenic region is located between the first polynucleotide and the second polynucleotide. In another aspect, the intergenic region located between the first polynucleotide and the second polynucleotide is a second intergenic region, with a first intergenic region included in the first polynucleotide as described below. As used herein, the terms “intergenic region” and intergenic sequence” can be used interchangeably, unless context clearly indicates otherwise.

An intergenic region located between the first polynucleotide and the second polynucleotide can be of any length and can have any nucleotide sequence. As an example, the intergenic region between the first polynucleotide and the second polynucleotide can include about one nucleotide, about two nucleotides, about three nucleotides, about four nucleotides, about five nucleotides, about six nucleotides, about seven nucleotides, about eight nucleotides, about nine nucleotides, about ten nucleotides, about 11 nucleotides, about 12 nucleotides, about 13 nucleotides, about 14 nucleotides, about 15 nucleotides, about 16 nucleotides, about 17 nucleotides, about 18 nucleotides, about 19 nucleotides, about 20 nucleotides, about 21 nucleotides, about 22 nucleotides, about 23 nucleotides, about 24 nucleotides, about 25 nucleotides, about 26 nucleotides, about 27 nucleotides, about 28 nucleotides, about 29 nucleotides, about 30 nucleotides, about 31 nucleotides, about 32 nucleotides, about 33 nucleotides, about 34 nucleotides, about 35 nucleotides, about 36 nucleotides, about 37 nucleotides, about 38 nucleotides, about 39 nucleotides, about 40 nucleotides, about 41 nucleotides, about 42 nucleotides, about 43 nucleotides, about 44 nucleotides, about 45 nucleotides, about 46 nucleotides, about 47 nucleotides, about 48 nucleotides, about 49 nucleotides, about 50 nucleotides, about 60 nucleotides, about 70 nucleotides, about 80 nucleotides, about 90 nucleotides, about 100 nucleotides, about 125 nucleotides, about 150 nucleotides, about 175 nucleotides, about 200 nucleotides, about 250 nucleotides, about 300 nucleotides, about 350 nucleotides, about 400 nucleotides, about 450 nucleotides, about 500 nucleotides, about 600 nucleotides, about 700 nucleotides, about 800 nucleotides, about 1,000 nucleotides, about 1,500 nucleotides, about 2,000 nucleotides, about 2,500 nucleotides, about 3,000 nucleotides, about 3,500 nucleotides, about 4,000 nucleotides, about 4,500 nucleotides, about 5,000 nucleotides, about 6,000 nucleotides, about 7,000 nucleotides, about 8,000 nucleotides, about 9,000 nucleotides, about 10,000 nucleotides, and any number or range in between. In one aspect, the intergenic region between first and second polynucleotides includes about 10-100 nucleotides, about 10-200 nucleotides, about 10-300 nucleotides, about 10-400 nucleotides, or about 10-500 nucleotides. In another aspect, the intergenic region between first and second polynucleotides includes about 1-10 nucleotides, about 1-20 nucleotides, about 1-30 nucleotides, about 1-40 nucleotides, or about 1-50 nucleotides. In yet another aspect, the region includes about 44 nucleotides. In one aspect, the intergenic region between first and second polynucleotides of nucleic acid molecules provided herein is a second intergenic region.

In one aspect, the intergenic region between first and second polynucleotides includes a viral sequence. The intergenic region between first and second polynucleotides can include a sequence from any virus, such as alphaviruses and rubiviruses, for example. In one aspect, the intergenic region between the first polynucleotide and the second polynucleotide comprises an alphavirus sequence, such as a sequence from Venezuelan Equine Encephalitis Virus (VEEV), Eastern Equine Encephalitis Virus (EEEV), Everglades Virus (EVEV), Mucambo Virus (MUCV), Semliki Forest Virus (SFV), Pixuna Virus (PIXV), Middleburg Virus (MIDV), Chikungunya Virus (CHIKV), ONyong-Nyong Virus (ONNV), Ross River Virus (RRV), Barmah Forest Virus (BFV), Getah Virus (GETV), Sagiyama Virus (SAGV), Bebaru Virus (BEBV), Mayaro Virus (MAYV), Una Virus (UNAV), Sindbis Virus (STNV), Aura Virus (AURAV), Whataroa Virus (WHAV), Babanki Virus (BABV), Kyzylagach Virus (KYZV), Western Equine Encephalitis Virus (WEEV), Highland J Virus (HJV), Fort Morgan Virus (FMV), Ndumu Virus (NDUV), Salmonid Alphavirus (SAV), Buggy Creek Virus (BCRV), or any combination thereof. In another aspect, the intergenic region between first and second polynucleotides comprises a sequence from Venezuelan Equine Encephalitis Virus (VEEV). In yet another aspect, the intergenic region between first and second polynucleotides comprises a sequence having at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9%, and any number or range in between, identity to SEQ ID NO:77. In a further aspect, the intergenic region between first and second polynucleotides comprises a sequence of SEQ ID NO:77. In yet a further aspect, the intergenic region between first and second polynucleotides is a second intergenic region comprising a sequence having at least 85% identity to SEQ ID NO:77.

Natural and Modified Nucleotides

A self-replicating RNA of the disclosure can comprise one or more chemically modified nucleotides. Examples of nucleic acid monomers include non-natural, modified, and chemically-modified nucleotides, including any such nucleotides known in the art. Nucleotides can be artificially modified at either the base portion or the sugar portion. In nature, most polynucleotides comprise nucleotides that are “unmodified” or “natural” nucleotides, which include the purine bases adenine (A) and guanine (G), and the pyrimidine bases thymine (T), cytosine (C) and uracil (U). These bases are typically fixed to a ribose or deoxy ribose at the 1′ position. The use of RNA polynucleotides comprising chemically modified nucleotides have been shown to improve RNA expression, expression rates, half-life and/or expressed protein concentrations. RNA polynucleotides comprising chemically modified nucleotides have also been useful in optimizing protein localization thereby avoiding deleterious bio-responses such as immune responses and/or degradation pathways.

Examples of modified or chemically-modified nucleotides include 5-hydroxycytidines, 5-alkylcytidines, 5-hydroxyalkylcytidines, 5-carboxycytidines, 5-formylcytidines, 5-alkoxycytidines, 5-alkynylcytidines, 5-halocytidines, 2-thiocytidines, N4-alkylcytidines, N4-aminocytidines, N4-acetylcytidines, and N4,N4-dialkylcytidines.

Examples of modified or chemically-modified nucleotides include 5-hydroxycytidine, 5-methylcytidine, 5-hydroxymethylcytidine, 5-carboxycytidine, 5-formylcytidine, 5-methoxycytidine, 5-propynylcytidine, 5-bromocytidine, 5-iodocytidine, 2-thiocytidine; N4-methylcytidine, N4-aminocytidine, N4-acetylcytidine, and N4,N4-dimethylcytidine.

Examples of modified or chemically-modified nucleotides include 5-hydroxyuridines, 5-alkyluridines, 5-hydroxyalkyluridines, 5-carboxyuridines, 5-carboxyalkylesteruridines, 5-formyluridines, 5-alkoxyuridines, 5-alkynyluridines, 5-halouridines, 2-thiouridines, and 6-alkyluridines.

Examples of modified or chemically-modified nucleotides include 5-hydroxyuridine, 5-methyluridine, 5-hydroxymethyluridine, 5-carboxyuridine, 5-carboxymethylesteruridine, 5-formyluridine, 5-methoxyuridine (also referred to herein as “5MeOU”), 5-propynyluridine, 5-bromouridine, 5-fluorouridine, 5-iodouridine, 2-thiouridine, and 6-methyluridine.

Examples of modified or chemically-modified nucleotides include 5-methoxycarbonylmethyl-2-thiouridine, 5-methylaminomethyl-2-thiouridine, 5-carbamoylmethyluridine, 5-carbamoylmethyl-2′-O-methyluridine, 1-methyl-3-(3-amino-3-carboxypropy)pseudouridine, 5-methylaminomethyl-2-selenouridine, 5-carboxymethyluridine, 5-methyldihydrouridine, 5-taurinomethyluridine, 5-taurinomethyl-2-thiouridine, 5-(isopentenylaminomethyl)uridine, 2′-O-methylpseudouridine, 2-thio-2′O-methyluridine, and 3,2′-O-dimethyluridine.

Examples of modified or chemically-modified nucleotides include N6-methyladenosine, 2-aminoadenosine, 3-methyladenosine, 8-azaadenosine, 7-deazaadenosine, 8-oxoadenosine, 8-bromoadenosine, 2-methylthio-N6-methyladenosine, N6-isopentenyladenosine, 2-methylthio-N6-isopentenyladenosine, N6-(cis-hydroxyisopentenyl)adenosine, 2-methylthio-N6-(cis-hydroxyisopentenyl)adenosine, N6-glycinylcarbamoyladenosine, N6-threonylcarbamoyl-adenosine, N6-methyl-N6-threonylcarbamoyl-adenosine, 2-methylthio-N6-threonylcarbamoyl-adenosine, N6,N6-dimethyladenosine, N6-hydroxynorvalylcarbamoyladenosine, 2-methylthio-N6-hydroxynorvalylcarbamoyl-adenosine, N6-acetyl-adenosine, 7-methyl-adenine, 2-methylthio-adenine, 2-methoxy-adenine, alpha-thio-adenosine, 2′-O-methyl-adenosine, N6,2′-O-dimethyl-adenosine, N6,N6,2′-O-trimethyl-adenosine, 1,2′-O-dimethyl-adenosine, 2′-O-ribosyladenosine, 2-amino-N6-methyl-purine, 1-thio-adenosine, 2′-F-ara-adenosine, 2′-F-adenosine, 2′-OH-ara-adenosine, and N6-(19-amino-pentaoxanonadecyl)-adenosine.

Examples of modified or chemically-modified nucleotides include N1-alkylguanosines, N2-alkylguanosines, thienoguanosines, 7-deazaguanosines, 8-oxoguanosines, 8-bromoguanosines, 06-alkylguanosines, xanthosines, inosines, and N1-alkylinosines.

Examples of modified or chemically-modified nucleotides include N1-methylguanosine, N2-methylguanosine, thienoguanosine, 7-deazaguanosine, 8-oxoguanosine, 8-bromoguanosine, 06-methylguanosine, xanthosine, inosine, and N1-methylinosine.

Examples of modified or chemically-modified nucleotides include pseudouridines. Examples of pseudouridines include Nhalkylpseudouridines, N1-cycloalkylpseudouridines, N1-hydroxypseudouridines, N1-hydroxyalkylpseudouridines, N1-phenylpseudouridines, N1-phenylalkylpseudouridines, N1-aminoalkylpseudouridines, N3-alkylpseudouridines, N6-alkylpseudouridines, N6-alkoxypseudouridines, N6-hydroxypseudouridines, N6-hydroxyalkylpseudouridines, N6-morpholinopseudouridines, N6-phenylpseudouridines, and N6-halopseudouridines. Examples of pseudouridines include N1-alkyl-N6-alkylpseudouridines, N1-alkyl-N6-alkoxypseudouridines, N1-alkyl-N6-hydroxypseudouridines, N1-alkyl-N6-hydroxyalkylpseudouridines, N1-alkyl-N6-morpholinopseudouridines, N1-alkyl-N6-phenylpseudouridines, and N1-alkyl-N6-halopseudouridines. In these examples, the alkyl, cycloalkyl, and phenyl substituents may be unsubstituted, or further substituted with alkyl, halo, haloalkyl, amino, or nitro substituents.

Examples of pseudouridines include N1-methylpseudouridine (also referred to herein as “N1MPU”),N1-ethylpseudouridine, N1-propylpseudouridine, N1-cyclopropylpseudouridine, N1-phenylpseudouridine, N1-aminomethylpseudouridine, N3-methylpseudouridine, N1-hydroxypseudouridine, and N1-hydroxymethylpseudouridine.

Examples of nucleic acid monomers include modified and chemically-modified nucleotides, including any such nucleotides known in the art.

Examples of modified and chemically-modified nucleotide monomers include any such nucleotides known in the art, for example, 2′-O-methyl ribonucleotides, 2′-O-methyl purine nucleotides, 2′-deoxy-2′-fluoro ribonucleotides, 2′-deoxy-2′-fluoro pyrimidine nucleotides, 2′-deoxy ribonucleotides, 2′-deoxy purine nucleotides, universal base nucleotides, 5-C-methyl-nucleotides, and inverted deoxyabasic monomer residues.

Examples of modified and chemically-modified nucleotide monomers include 3′-end stabilized nucleotides, 3′-glyceryl nucleotides, 3′-inverted abasic nucleotides, and 3′-inverted thymidine.

Examples of modified and chemically-modified nucleotide monomers include locked nucleic acid nucleotides (LNA), 2′-O,4′-C-methylene-(D-ribofuranosyl) nucleotides, 2′-methoxyethoxy (MOE) nucleotides, 2′-methyl-thio-ethyl, 2′-deoxy-2′-fluoro nucleotides, and 2′-O-methyl nucleotides. In an exemplary embodiment, the modified monomer is a locked nucleic acid nucleotide (LNA).

Examples of modified and chemically-modified nucleotide monomers include 2′,4′-constrained 2′-O-methoxyethyl (cMOE) and 2′-O-Ethyl (cEt) modified DNAs.

Examples of modified and chemically-modified nucleotide monomers include 2′-amino nucleotides, 2′-O-amino nucleotides, 2′-C-allyl nucleotides, and 2′-O-allyl nucleotides.

Examples of modified and chemically-modified nucleotide monomers include N6-methyladenosine nucleotides.

Examples of modified and chemically-modified nucleotide monomers include nucleotide monomers with modified bases 5-(3-amino)propyluridine, 5-(2-mercapto)ethyluridine, 5-bromouridine; 8-bromoguanosine, or 7-deazaadenosine.

Examples of modified and chemically-modified nucleotide monomers include 2′-O-aminopropyl substituted nucleotides.

Examples of modified and chemically-modified nucleotide monomers include replacing the 2′-OH group of a nucleotide with a 2′-R, a 2′-OR, a 2′-halogen, a 2′-SR, or a 2′-amino, where R can be H, alkyl, alkenyl, or alkynyl.

Example of base modifications described above can be combined with additional modifications of nucleoside or nucleotide structure, including sugar modifications and linkage modifications. Certain modified or chemically-modified nucleotide monomers may be found in nature.

Preferred nucleotide modifications include N1-methylpseudouridine and 5-methoxyuridine.

Viral Replication Proteins and Polynucleotides Encoding Them

Provided herein, in some embodiments, are nucleic acid molecules comprising a first polynucleotide encoding one or more viral replication proteins. As used herein, the term “replication protein” or “viral replication protein” refers to any protein or any protein subunit of a protein complex that functions in replication of a viral genome. Generally, viral replication proteins are non-structural proteins. Viral replication proteins encoded by nucleic acid molecules provided herein can function in the replication of any viral genome. The viral genome can be a single-stranded positive-sense RNA genome, a single-stranded negative-sense RNA genome, a double-stranded RNA genome, a single-stranded positive-sense DNA genome, a single-stranded negative-sense DNA genome, or a double-stranded DNA genome. Viral genomes can include a single nucleic acid molecule or more than one nucleic acid molecule. Nucleic acid molecules provided herein can encode one or more viral replication proteins from any virus or virus family, including animal viruses and plant viruses, for example. Viral replication proteins encoded by first polynucleotides included in nucleic acid molecules provided herein can be expressed from self-replicating RNA.

First polynucleotide sequences of nucleic acid molecules provided herein can encode one or more togavirus replication proteins. In some aspects, the one or more viral replication proteins encoded by first polynucleotides of nucleic acid molecules provided herein are alphavirus proteins. In some embodiments, the one or more viral replication proteins encoded by first polynucleotides of nucleic acid molecules provided herein are rubivirus proteins. First polynucleotide sequences of nucleic acid molecules provided herein can encode any alphavirus replication protein and any rubivirus replication protein. Exemplary replication proteins from alphaviruses include proteins from Venezuelan Equine Encephalitis Virus (VEEV), Eastern Equine Encephalitis Virus (EEEV), Everglades Virus (EVEV), Mucambo Virus (MUCV), Semliki Forest Virus (SFV), Pixuna Virus (PIXV), Middleburg Virus (MIDV), Chikungunya Virus (CHIKV), O'Nyong-Nyong Virus (ONNV), Ross River Virus (RRV), Barmah Forest Virus (BFV), Getah Virus (GETV), Sagiyama Virus (SAGV), Bebaru Virus (BEBV), Mayaro Virus (MAYV), Una Virus (UNAV), Sindbis Virus (SINV), Aura Virus (AURAV), Whataroa Virus (WHAV), Babanki Virus (BABV), Kyzylagach Virus (KYZV), Western Equine Encephalitis Virus (WEEV), Highland J Virus (HJV), Fort Morgan Virus (FMV), Ndumu Virus (NDUV), Salmonid Alphavirus (SAV), Buggy Creek Virus (BCRV), and any combination thereof. Exemplary rubivirus replication proteins include proteins from rubella virus.

Viral replication proteins encoded by first polynucleotides of nucleic acid molecules provided herein can be expressed as one or more polyproteins or as separate or single proteins. Generally, polyproteins are precursor proteins that are cleaved to generate individual or separate proteins. Accordingly, proteins derived from a precursor polyprotein can be expressed from a single open reading frame (ORF). As used herein, the term “ORF” refers to a nucleotide sequence that begins with a start codon, generally ATG, and that ends with a stop codon, such as TAA, TAG, or TGA, for example. It will be appreciated that T is present in DNA, while U is present in RNA. Accordingly, a start codon of ATG in DNA corresponds to AUG in RNA, and the stop codons TAA, TAG, and TGA in DNA correspond to UAA, UAG, and UGA in RNA. It will further be appreciated that for any sequence provided in the present disclosure, T is present in DNA, while U is present in RNA. Accordingly, for any sequence provided herein, T present in DNA is substituted with U for an RNA molecule, and U present in RNA is substituted with T for a DNA molecule.

The protease cleaving a polyprotein can be a viral protease or a cellular protease. In some aspects, the first polynucleotide of nucleic acid molecules provided herein encodes a polyprotein comprising an alphavirus nsP1 protein, an alphavirus nsP2 protein, an alphavirus nsP3 protein, an alphavirus nsP4 protein, or any combination thereof. In other aspects, the first polynucleotide of nucleic acid molecules provided herein encodes a polyprotein comprising an alphavirus nsP1 protein, an alphavirus nsP2 protein, an alphavirus nsP3 protein, or any combination thereof, and an alphavirus nsP4 protein. In some aspects, the polyprotein is a VEEV polyprotein. In other aspects, the alphavirus nsP1, nsP2, nsP3, and nsP4 proteins are VEEV proteins.

In one aspect, first polynucleotides of nucleic acid molecules provided herein lack a stop codon between sequences encoding an nsP3 protein and an nsP4 protein. Accordingly, in some aspects, first polynucleotides of nucleic acid molecules provided herein encode a P1234 polyprotein comprising nsP1, nsP2, nsP3, and nsP4. First polynucleotides of nucleic acid molecules provided herein can also include a stop codon between sequences encoding an nsP3 and an nsP4 protein. Accordingly, in some aspects, first polynucleotides of nucleic acid molecules provided herein encode a P123 polyprotein comprising nsP1, nsP2, and nsP3 and a P1234 polyprotein comprising nsP1, nsP2, nsP3, and nsP4 as a result of stop codon readthrough, for example. In other aspects, first polynucleotides of nucleic acid molecules provided herein encode a polyprotein having at least 80%, at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9%, and any number or range in between, identity to a sequence of SEQ ID NO:79. In some embodiments, first polynucleotides of nucleic acid molecules provided herein encode a polyprotein having a sequence of SEQ ID NO:79. Further exemplary polyproteins comprise a sequence of SEQ ID NO:80 or SEQ ID NO:81. In one aspect, nsP2 and nsP3 proteins include mutations. Exemplary mutations include G1309R and 51583G mutations of VEEV proteins. In another aspect, the nsP1, nsP2, and nsP4 proteins are VEEV proteins, and the nsP3 protein is a chikungunya virus (CHIKV) nsP3 protein.

In some aspects, first polynucleotides of nucleic acid molecules provided herein can include a first intergenic region. In some aspects, the first intergenic region is located between a sequence encoding a polyprotein comprising an alphavirus nsP1 protein, an alphavirus nsP2 protein, an alphavirus nsP3 protein, or any combination thereof, and a sequence encoding an alphavirus nsP4 protein. A first intergenic region can comprise any sequence, such as any viral or non-viral sequence. In one aspect, the first intergenic region comprises a viral sequence. In another aspect, the first intergenic region comprises an alphavirus sequence. In yet another aspect, the alphavirus is VEEV. In one aspect, nsP2 and nsP3 proteins include mutations. Exemplary mutations include G1309R and S1583G mutations of VEEV proteins. In another aspect, the nsP1, nsP2, and nsP4 proteins are VEEV proteins, and the nsP3 protein is a chikungunya virus (CHIKV) nsP3 protein.

5′ Untranslated Region (5′ UTR)

Nucleic acid molecules provided herein can further comprise untranslated regions (UTRs). Untranslated regions, including 5′ UTRs and 3′ UTRs, for example, can affect RNA stability and/or efficiency of RNA translation, such as translation of cellular and viral mRNAs, for example. 5′ UTRs and 3′ UTRs can also affect stability and translation of viral genomic RNAs and self-replicating RNAs, including virally derived self-replicating RNAs or replicons. Exemplary viral genomic RNAs whose stability and/or efficiency of translation can be affected by 5′ UTRs and 3′ UTRs include the genome nucleic acid of positive-sense RNA viruses. Both genome nucleic acid of positive-sense RNA viruses and self-replicating RNAs, including virally derived self-replicating RNAs or replicons, can be translated upon infection or introduction into a cell.

In some aspects, nucleic acid molecules provided herein further include a 5′ untranslated region (5′ UTR). Any 5′ UTR sequence can be included in nucleic acid molecules provided herein. In some embodiments, nucleic acid molecules provided herein include a viral 5′ UTR. In one aspect, nucleic acid molecules provided herein include a non-viral 5′ UTR. Any non-viral 5′ UTR can be included in nucleic acid molecules provided herein, such as 5′ UTRs of transcripts expressed in any cell or organ, including muscle, skin, subcutaneous tissue, liver, spleen, lymph nodes, antigen-presenting cells, and others. In another aspect, nucleic acid molecules provided herein include a 5′ UTR comprising viral and non-viral sequences. Accordingly, a 5′ UTR included in nucleic acid molecules provided herein can comprise a combination of viral and non-viral 5′ UTR sequences. In some aspects, the 5′ UTR included in nucleic acid molecules provided herein is located upstream of or 5′ of the first polynucleotide that encodes one or more viral replication proteins. In other aspects, the 5′ UTR is located 5′ of or upstream of the first polynucleotide of nucleic acid molecules provided herein that encodes one or more viral replication proteins, and the first polynucleotide is located 5′ of or upstream of the second polynucleotide of nucleic acid molecules provided herein.

In one aspect, the 5′ UTR of nucleic acid molecules provided herein comprises an alphavirus 5′ UTR. A 5′ UTR from any alphavirus can be included in nucleic acid molecules provided herein, including 5′ UTR sequences from Venezuelan Equine Encephalitis Virus (VEEV), Eastern Equine Encephalitis Virus (EEEV), Everglades Virus (EVEV), Mucambo Virus (MUCV), Semliki Forest Virus (SFV), Pixuna Virus (PIXV), Middleburg Virus (MIDV), Chikungunya Virus (CHIKV), O'Nyong-Nyong Virus (ONNV), Ross River Virus (RRV), Barmah Forest Virus (BFV), Getah Virus (GETV), Sagiyama Virus (SAGV), Bebaru Virus (BEBV), Mayaro Virus (MAYV), Una Virus (UNAV), Sindbis Virus (STNV), Aura Virus (AURAV), Whataroa Virus (WHAV), Babanki Virus (BABV), Kyzylagach Virus (KYZV), Western Equine Encephalitis Virus (WEEV), Highland J Virus (HJV), Fort Morgan Virus (FMV), Ndumu Virus (NDUV), Salmonid Alphavirus (SAV), or Buggy Creek Virus (BCRV). In another aspect, the 5′ UTR comprises a sequence having at least 80%, at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9%, and any number or range in between, identity to a sequence of SEQ ID NO:73, SEQ ID NO:74, or SEQ ID NO:75. In yet another aspect, the 5′ UTR comprises a sequence of SEQ ID NO:73, SEQ ID NO:74, or SEQ ID NO:75.

In some embodiments, the 5′ UTR comprises a sequence selected from the 5′ UTRs of human IL-6, alanine aminotransferase 1, human apolipoprotein E, human fibrinogen alpha chain, human transthyretin, human haptoglobin, human alpha-1-antichymotrypsin, human antithrombin, human alpha-1-antitrypsin, human albumin, human beta globin, human complement C3, human complement C5, SynK (thylakoid potassium channel protein derived from the cyanobacteria, Synechocystis sp.), mouse beta globin, mouse albumin, and a tobacco etch virus, or fragments of any of the foregoing. Preferably, the 5′ UTR is derived from a tobacco etch virus (TEV). Preferably, an mRNA described herein comprises a 5′ UTR sequence that is derived from a gene expressed by Arabidopsis thaliana. Preferably, the 5′ UTR sequence of a gene expressed by Arabidopsis thaliana is AT1G58420. Examples of 5 UTRs and 3′ UTRs are described in PCT/US2018/035419, the contents of which are herein incorporated by reference. Preferred 5′ UTR sequences comprise SEQ ID NOs: 5, 25-27 and 28-45: as shown in Table 1.

TABLE 1
5′ UTR Sequences

| Name | Sequence | Seq ID No.: |
|---|---|---|
| EV | UCAACACAACAUAUACAAAACAAACGAAUCUCAAGCAAUC | SEQ ID NO: 5 |
| | AAGCAUUCUACUUCUAUUGCAGCAAUUUAAAUCAUUUCU | |
| | UUUAAAGCAAAAGCAAUUUUCUGAAAAUUUUCACCAUUU | |
| | ACGAACGAUAG | |
| AT1G58420 | AUUAUUACAUCAAAACAAAAAGCCGCCA | SEQ ID NO: 6 |
| ARC5-2 | CUUAAGGGGGCGCUGCCUACGGAGGUGGCAGCCAUCUCCU | SEQ ID NO: 7 |
| | UCUCGGCAUCAAGCUUACCAUGGUGCCCCAGGCCCUGCUC | |
| | UUGGUCCCGCUGCUGGUGUUCCCCCUCUGCUUCGGCAAGU | |
| | UCCCCAUCUACACCAUCCCCGACAAGCUGGGGCCGUGGAG | |
| | CCCCAUCGACAUCCACCACCUGUCCUGCCCCAACAACCUCG | |
| | UGGUCGAGGACGAGGGCUGCACCAACCUGAGCGGGUUCUC | |
| | CUAC | |
| HCV | UGAGUGUCGU ACAGCCUCCA GGCCCCCCCC | SEQ ID NO: 8 |
| | UCCCGGGAGA GCCAUAGUGG | |
| | UCUGCGGAACCGGUGAGUAC ACCGGAAUUG | |
| | CCGGGAAGAC UGGGUCCUUU CUUGGAUAAA | |
| | CCCACUCUAUGCCCGGCCAU UUGGGCGUGC | |
| | CCCCGCAAGA CUGCUAGCCG AGUAGUGUUG GGUUGCG | |
| HUMAN | AAUUAUUGGUUAAAGAAGUAUAUUAGUGCUAAUUUCCCU | SEQ ID NO: 9 |
| ALBUMIN | CCGUUUGUCCUAGCUUUUCUCUUCUGUCAACCCCACACGC | |
| | CUUUGGCACA | |
| EMCV | CUCCCUCCCC CCCCCCUAAC GUUACUGGCC | SEQ ID NO: 10 |
| | GAAGCCGCUU GGAAUAAGGC CGGUGUGCGU | |
| | UUGUCUAUAU GUUAUUUUCC ACCAUAUUGC | |
| | CGUCUUUUGG CAAUGUGAGG GCCCGGAAAC | |
| | CUGGCCCUGU CUUCUUGACG AGCAUUCCUA | |
| | GGGGUCUUUC CCCUCUCGCC AAAGGAAUGC | |
| | AAGGUCUGUU GAAUGUCGUG AAGGAAGCAG | |
| | UUCCUCUGGA AGCUUCUUGA AGACAAACAA | |
| | CGUCUGUAGC GACCCUUUGC AGGCAGCGGA | |
| | ACCCCCCACC UGGCGACAGG UGCCUCUGCG | |
| | GCCAAAAGCC ACGUGUAUAA GAUACACCUG | |
| | CAAAGGCGGC ACAACCCCAG UGCCACGUUG | |
| | UGAGUUGGAU AGUUGUGGAA AGAGUCAAAU | |
| | GGCUCUCCUC AAGCGUAUUC AACAAGGGGC | |
| | UGAAGGAUGC CCAGAAGGUA CCCCAUUGUA | |
| | UGGGAUCUGA UCUGGGGCCU CGGUGCACAU | |
| | GCUUUACGUG UGUUUAGUCG AGGUUAAAAA | |
| | ACGUCUAGGC CCCCCGAACC ACGGGGACGU | |
| | GGUUUUCCUU UGAAAAACAC GAUGAUAAU | |
| AT1G67090 | CACAAAGAGUAAAGAAGAACA | SEQ ID NO: 25 |
| AT1G35720 | AACACUAAAAGUAGAAGAAAA | SEq ID NO: 26 |
| AT5G45900 | CUCAGAAAGAUAAGAUCAGCC | SEQ ID NO: 27 |
| AT5G61250 | AACCAAUCGAAAGAAACCAAA | SEQ ID NO: 28 |
| AT5G46430 | CUCUAAUCACCAGGAGUAAAA | SEQ ID NO: 29 |
| AT5G47110 | GAGAGAGAUCUUAACAAAAAA | SEQ ID NO: 30 |
| AT1G03110 | UGUGUAACAACAACAACAACA | SEQ ID NO: 31 |
| AT3G12380 | CCGCAGUAGGAAGAGAAAGCC | SEQ ID NO: 32 |
| AT5G45910 | AAAAAAAAAAGAAAUCAUAAA | SEQ ID NO: 33 |
| AT1G07260 | GAGAGAAGAAAGAAGAAGACG | SEQ ID NO: 34 |
| AT3G55500 | CAAUUAAAAAUACUUACCAAA | SEQ ID NO: 35 |
| AT3G46230 | GCAAACAGAGUAAGCGAAACG | SEQ ID NO: 36 |
| AT2G36170 | GCGAAGAAGACGAACGCAAAG | SEQ ID NO: 37 |
| AT1G10660 | UUAGGACUGUAUUGACUGGCC | SEQ ID NO: 38 |
| AT4G14340 | AUCAUCGGAAUUCGGAAAAAG | SEQ ID NO: 39 |
| AT1G49310 | AAAACAAAAGUUAAAGCAGAC | SEQ ID NO: 40 |
| AT4G14360 | UUUAUCUCAAAUAAGAAGGCA | SEQ ID NO: 41 |
| AT1G28520 | GGUGGGGAGGUGAGAUUUCUU | SEQ ID NO: 42 |
| AT1G20160 | UGAUUAGGAAACUACAAAGCC | SEQ ID NO: 43 |
| AT5G37370 | CAUUUUUCAAUUUCAUAAAAC | SEQ ID NO: 44 |
| AT4G11320 | UUACUUUUAAGCCCAACAAAA | SEQ ID NO: 45 |
| AT5G40850 | GGCGUGUGUGUGUGUUGUUGA | SEQ ID NO: 46 |
| AT1G06150 | GUGGUGAAGGGGAAGGUUUAG | SEQ ID NO: 47 |
| AT2G26080 | UUGUUUUUUUUUGGUUUGGUU | SEQ ID NO: 48 |

3′ Untranslated Region (3′ UTR)

In some aspects, nucleic acid molecules provided herein further include a 3′ untranslated region (3′ UTR). Any 3′ UTR sequence can be included in nucleic acid molecules provided herein. In one aspect, nucleic acid molecules provided herein include a viral 3′ UTR. In another aspect, nucleic acid molecules provided herein include a non-viral 3′ UTR. Any non-viral 3′ UTR can be included in nucleic acid molecules provided herein, such as 3′ UTRs of transcripts expressed in any cell or organ, including muscle, skin, subcutaneous tissue, liver, spleen, lymph nodes, antigen-presenting cells, and others. In some aspects, nucleic acid molecules provided herein include a 3′ UTR comprising viral and non-viral sequences. Accordingly, a 3′ UTR included in nucleic acid molecules provided herein can comprise a combination of viral and non-viral 3′ UTR sequences. In one aspect, the 3′ UTR is located 3′ of or downstream of the second polynucleotide of nucleic acid molecules provided herein that comprises a first transgene encoding a first antigenic protein or a fragment thereof. In another aspect, the 3′ UTR is located 3′ of or downstream of the second polynucleotide of nucleic acid molecules provided herein that comprises a first transgene encoding a first antigenic protein or a fragment thereof, and the second polynucleotide is located 3′ of or downstream of the first polynucleotide of nucleic acid molecules provided herein.

In one aspect, the 3′ UTR of nucleic acid molecules provided herein comprises an alphavirus 3′ UTR. A 3′ UTR from any alphavirus can be included in nucleic acid molecules provided herein, including 3′ UTR sequences from Venezuelan Equine Encephalitis Virus (VEEV), Eastern Equine Encephalitis Virus (EEEV), Everglades Virus (EVEV), Mucambo Virus (MUCV), Semliki Forest Virus (SFV), Pixuna Virus (PIXY), Middleburg Virus (MIDV), Chikungunya Virus (CHIKV), O'Nyong-Nyong Virus (ONNV), Ross River Virus (RRV), Barmah Forest Virus (BFV), Getah Virus (GETV), Sagiyama Virus (SAGV), Bebaru Virus (BEBV), Mayaro Virus (MAYV), Una Virus (UNAV), Sindbis Virus (SINV), Aura Virus (AURAV), Whataroa Virus (WHAV), Babanki Virus (BABV), Kyzylagach Virus (KYZV), Western Equine Encephalitis Virus (WEEV), Highland J Virus (HJV), Fort Morgan Virus (FMV), Ndumu Virus (NDUV), Salmonid Alphavirus (SAV), or Buggy Creek Virus (BCRV). In another aspect, the 3′ UTR comprises a sequence having at least 80%, at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9%, and any number or range in between, identity to a sequence of SEQ ID NO:5. In yet another aspect, the 3′ UTR comprises a poly-A sequence. In a further aspect, the 3′ UTR comprises a sequence of SEQ ID NO:5.

In some embodiments, the 3′ UTR comprises a sequence selected from the 3′ UTRs of alanine aminotransferase 1, human apolipoprotein E, human fibrinogen alpha chain, human haptoglobin, human antithrombin, human alpha globin, human beta globin, human complement C3, human growth factor, human hepcidin, MALAT-1, mouse beta globin, mouse albumin, and Xenopus beta globin, or fragments of any of the foregoing. In some embodiments, the 3′ UTR is derived from Xenopus beta globin. Exemplary 3′ UTR sequences include SEQ ID NOs: 16-22 as shown in Table 2.

TABLE 2
3′ UTR sequences.

| Name | Sequence | Seq ID No.: |
|---|---|---|
| XBG | CUAGUGACUGACUAGGAUCUGGUUACCACUAAACCAG | SEQ ID NO: 16 |
| | CCUCAAGAACACCCGAAUGGAGUCUCUAAGCUACAUA | |
| | AUACCAACUUACACUUACAAAAUGUUGUCCCCCAAAA | |
| | UGUAGCCAUUCGUAUCUGCUCCUAAUAAAAAGAAAGU | |
| | UUCUUCACAU | |
| HUMAN | UGCAAGGCUGGCCGGAAGCCCUUGCCUGAAAGCAAGA | SEQ ID NO: 17 |
| HAPTOGLOBIN | UUUCAGCCUGGAAGAGGGCAAAGUGGACGGGAGUGG | |
| | ACAGGAGUGGAUGCGAUAAGAUGUGGUUUGAAGCUG | |
| | AUGGGUGCCAGCCCUGCAUUGCUGAGUCAAUCAAUAA | |
| | AGAGCUUUCUUUUGACCCAU | |
| HUMAN | ACGCCGAAGCCUGCAGCCAUGCGACCCCACGCCACCCC | SEQ ID NO: 18 |
| APOLIPOPROTEINE | GUGCCUCCUGCCUCCGCGCAGCCUGCAGCGGGAGACC | |
| | CUGUCCCCGCCCCAGCCGUCCUCCUGGGGUGGACCCU | |
| | AGUUUAAUAAAGAUUCACCAAGUUUCACGCA | |
| HCV | UAGAGCGGCAAACCCUAGCUACACUCCAUAGCUAGUU | SEQ ID NO: 19 |
| | UCUUUUUUUUUUGUUUUUUUUUUUUUUUUUUUUUUU | |
| | UUUUUUUUUUUUUUUUCCUUUCUUUUCCUUCUUUUU | |
| | UUCCUCUUUUCUUGGUGGCUCCAUCUUAGCCCUAGUC | |
| | ACGGCUAGCUGUGAAAGGUCCGUGAGCCGCAUGACUG | |
| | CAGAGAGUGCCGUAACUGGUCUCUCUGCAGAUCAUGU | |
| MOUSE | ACACAUCACAACCACAACCUUCUCAGGCUACCCUGAG | SEQ ID NO: 20 |
| ALBUMIN | AAAAAAAGACAUGAAGACUCAGGACUCAUCUUUUCUG | |
| | UUGGUGUAAAAUCAACACCCUAAGGAACACAAAUUUC | |
| | UUUAAACAUUUGACUUCUUGUCUCUGUGCUGCAAUUA | |
| | AUAAAAAAUGGAAAGAAUCUAC | |
| HUMAN ALPHA | GCUGGAGCCUCGGUAGCCGUUCCUCCUGCCCGCUGGG | SEQ ID NO: 21 |
| GLOBIN | CCUCCCAACGGGCCCUCCUCCCCUCCUUGCACCGGCCC | |
| | UUCCUGGUCUUUGAAUAAAGUCUGAGUGGGCAGCA | |
| EMCV | UAGUGCAGUCAC UGGCACAACG CGUUGCCCGG | SEQ ID NO: 22 |
| | UAAGCCAAUC GGGUAUACAC | |
| | GGUCGUCAUACUGCAGACAG GGUUCUUCUA | |
| | CUUUGCAAGA UAGUCUAGAG UAGUAAAAUA | |
| | AAUAGUAUAAG | |

Triple Stop Codon

In some embodiments, the self-replicating RNA may comprise a sequence immediately downstream of a coding region (i.e., ORF) that creates a triple stop codon. A triple stop codon is a sequence of three consecutive stop codons. The triple stop codon can ensure total insulation of an expression cassette and may be incorporated to enhance the efficiency of translation. In some embodiments, a self-replicating RNA of the disclosure may comprise a triple combination of any of the sequences UAG, UGA, or UAA immediately downstream of a ORF described herein. The triple combination can be three of the same codons, three different codons, or any other permutation of the three stop codons.

Translation Enhancers and Kozak Sequences

For translation initiation, proper interactions between ribosomes and mRNAs must be established to determine the exact position of the translation initiation region. However, ribosomes also must dissociate from the translation initiation region to slide toward the downstream sequence during mRNA translation. Translation enhancers upstream from initiation sequences of mRNAs enhance the yields of protein biosynthesis. Several studies have investigated the effects of translation enhancers. In some embodiments, an mRNA described herein comprises a translation enhancer sequence. These translation enhancer sequences enhance the translation efficiency of a self-replicating RNA of the disclosure and thereby provide increased production of the protein encoded by the mRNA. The translation enhancer region may be located in the 5′ or 3′ UTR of an mRNA sequence. Examples of translation enhancer regions include naturally-occurring enhancer regions from the TEV 5′ UTR and the Xenopus beta-globin 3′ UTR. Exemplary 5′ UTR enhancer sequences include but are not limited to those derived from mRNAs encoding human heat shock proteins (HSP) including HSP70-P2, HSP70-M1 HSP72-M2, HSP17.9 and HSP70-P1. Preferred translation enhancer sequences used in accordance with the embodiments of the present disclosure are represented by SEQ ID Nos: 11-15 as shown in Table 3.

TABLE 3
5′ UTR Enhancers

| Name | Sequence | Seq ID No.: |
|---|---|---|
| HSP70-P2 | GUCAGCUUUCAAACUCUUUGUUUCUUGUUUGUUGAUUGAGAA | SEQ ID NO: 11 |
| | UA | |
| HSP70-M1 | CUCUCGCCUGAGAAAAAAAAUCCACGAACCAAUUUCUCAGCA | SEQ ID NO: 12 |
| | ACCAGCAGCACG | |
| HSP72-M2 | ACCUGUGAGGGUUCGAAGGAAGUAGCAGUGUUUUUUGUUCCU | SEQ ID NO: 13 |
| | AGAGGAAGAG | |
| HSP17.9 | ACACAGAAACAUUCGCAAAAACAAAAUCCCAGUAUCAAAAUU | SEQ ID NO: 14 |
| | CUUCUCUUUUUUUCAUAUUUCGCAAAGAC | |
| HSP70-P1 | CAGAAAAAUUUGCUACAUUGUUUCACAAACUUCAAAUAUUAU | SEQ ID NO: 15 |
| | UCAUUUAUUU | |

In some embodiments, a self-replicating RNA of the disclosure comprises a Kozak sequence. As is understood in the art, a Kozak sequence is a short consensus sequence centered around the translational initiation site of eukaryotic mRNAs that allows for efficient initiation of translation of the mRNA. See, for example, Kozak, Marilyn (1988) Mol. and Cell Biol, 8:2737-2744; Kozak, Marilyn (1991) J. Biol. Chem, 266: 19867-19870; Kozak, Marilyn (1990) Proc Natl. Acad. Sci. USA, 87:8301-8305; and Kozak, Marilyn (1989) J. Cell Biol, 108:229-241. It ensures that a protein is correctly translated from the genetic message, mediating ribosome assembly and translation initiation. The ribosomal translation machinery recognizes the AUG initiation codon in the context of the Kozak sequence. A Kozak sequence may be inserted upstream of the coding sequence for the protein of interest, downstream of a 5′ UTR or inserted upstream of the coding sequence for the protein of interest and downstream of a 5′ UTR. In some embodiments, a self-replicating RNA described herein comprises a Kozak sequence having the amino acid sequence GCCACC (SEQ ID NO: 23). Preferably a self-replicating RNA described herein comprises a partial Kozak sequence “p” having the amino acid sequence GCCA (SEQ ID NO: 24).

Transgenes

Transgenes included in nucleic acid molecules provided herein can encode an antigenic protein or a fragment thereof. In some embodiments, second polynucleotides of nucleic acid molecules provided herein comprise a first transgene. A first transgene included in second polynucleotides of nucleic acid molecules provided herein can encode a first antigenic protein or a fragment thereof. A transgene included in second polynucleotides of nucleic acid molecules provided herein can comprise a sequence encoding the full amino acid sequence of an antigenic protein or a sequence encoding any suitable portion or fragment of the full amino acid sequence of an antigenic protein. Any antigenic protein can be encoded by transgenes included in nucleic acid molecules provided herein. In one aspect, the antigenic protein is a viral protein, a bacterial protein, a fungal protein, a protozoan protein, a parasite protein, or a tumor protein or tumor antigen. Transgenes included in nucleic acid molecules provided herein can be expressed from a subgenomic RNA.

In another embodiment, the antigenic protein, when administered to a mammalian subject, raises an immune response to a pathogen, optionally wherein the pathogen is bacterial, viral, fungal, protozoan, or cancerous. In some more particular embodiments, the antigenic protein is expressed on the outer surface of the pathogen; while in other more particular embodiments, the antigen may be a non-surface antigen, e.g., useful as a T-cell epitope. The immunogen may elicit an immune response against a pathogen (e.g. a bacterium, a virus, a fungus or a parasite) but, in some other embodiments, it elicits an immune response against an allergen or a tumor antigen. The immune response may comprise an antibody response (usually including IgG) and/or a cell mediated immune response. The polypeptide immunogen will typically elicit an immune response that recognizes the corresponding pathogen (or allergen or tumor) polypeptide, but in some embodiments, the polypeptide may act as a mimotope to elicit an immune response that recognizes a saccharide. The immunogen will typically be a surface polypeptide e.g. an adhesin, a hemagglutinin, an envelope glycoprotein, a spike glycoprotein, etc.

Any viral, bacterial, fungal, protozoan, parasite, or tumor protein can be encoded by transgenes included in nucleic acid molecules provided herein. A protein from any infectious agent can be encoded by transgenes included in nucleic acid molecules provided herein. As used herein, the term “infectious agent” refers to any agent capable of infecting an organism, including humans and animals, and causing disease or deterioration in health. The terms “infectious agent” and “infectious pathogen” may be used interchangeably, unless context clearly indicates otherwise.

In some aspects, the viral protein encoded by transgenes included in nucleic acid molecules provided herein is an orthomyxovirus protein, a paramyxovirus protein, a picornavirus protein, a flavivirus protein, a filovirus protein, a rhabdovirus protein, a togavirus protein, an arterivirus protein, a bunyavirus protein, an arenavirus protein, a reovirus protein, a bornavirus protein, a retrovirus protein, an adenovirus protein, a herpesvirus protein, a polyomavirus protein, a papillomavirus protein, a poxvirus protein, or a hepadnavirus protein. In other aspects, the antigenic protein is an influenza virus protein, a respiratory syncytial virus (RSV) protein, a human immunodeficiency virus (HIV) protein, a hepatitis C virus (HCV) protein, a cytomegalovirus (CMV) protein, a Lassa Fever Virus (LFV) protein, an Ebola Virus (EBOV) protein, a Mycobacterium protein, a Bacillus protein, a Yersinia protein, a Streptococcus protein, a Pseudomonas protein, a Shigella protein, a Campylobacter protein, a Salmonella protein, a Plasmodium protein, or a Toxoplasma protein.

In one aspect, the antigenic protein is from a prokaryotic organism, including gram positive bacteria, gram negative bacteria, or other bacteria, such as Bacillus (e.g., Bacillus anthracis), Mycobacterium (e.g., Mycobacterium tuberculosis, Mycobacterium Leprae), Shigella (e.g., Shigella sonnei, Shigella dysenteriae, Shigella flexneri), Helicobacter (e.g., Helicobacter pylori), Salmonella (e.g., Salmonella enterica, Salmonella typhi, Salmonella typhimurium), Neisseria (e.g., Neisseria gonorrhoeae, Neisseria meningitidis), Moraxella (e.g., Moraxella catarrhalis), Haemophilus (e.g., Haemophilus influenzae), Klebsiella (e.g., Klebsiella pneumoniae), Legionella (e.g., Legionella pneumophila), Pseudomonas (e.g., Pseudomonas aeruginosa), Acinetobacter (e.g., Acinetobacter baumannii), Listeria (e.g., Listeria monocytogenes), Staphylococcus (e.g., Staphylococcus aureus), Streptococcus (e.g., Streptococcus pneumoniae, Streptococcus pyogenes, Streptococcus agalactiae), Corynebacterium (e.g., Corynebacterium diphtheria), Clostridium (e.g., Clostridium botulinum, Clostridium tetani, Clostridium difficile), Chlamydia (e.g., Chlamydia pneumonia, Chlamydia trachomatis), Caphylobacter (e.g., Caphylobacter jejuni), Bordetella (e.g., Bordetella pertussis), Enterococcus (e.g., Enterococcus faecalis, Enterococcus faecum), Vibrio (e.g., Vibrio cholerae), Yersinia (e.g., Yersinia pestis), Burkholderia (e.g., Burkholderia cepacia complex), Coxiella (e.g., Coxiella burnetti), Francisella (e.g., Francisella tularensis), and Escherichia (e.g., enterotoxigenic, enterohemorrhagic or Shiga toxin producing E. coli, such as ETEC, EHEC, EPEC, EIEC, and EAEC)). In another aspect, the antigenic protein is from a eukaryotic organism, including protists and fungi, such as Plasmodium (e.g., Plasmodium falciparum, Plasmodium vivax, Plasmodium ovale, Plasmodium malariae, Plasmodium diarrhea), Candida (e.g., Candida albicans), Aspergillus (e.g., Aspergillus fumigatus), Cryptococcus (e.g., Cryptococcus neoformans), Histoplasma (e.g., Histoplasma capsulatum), Pneumocystis (e.g., Pneumocystis jirovecii), and Coccidiodes (e.g., Coccidiodes immitis).

In one aspect, the antigenic protein encoded by first transgenes of second polynucleotides included in nucleic acid molecules provided herein is an influenza virus protein or a fragment thereof. In another aspect, the second polynucleotide includes one or more transgenes encoding one or more influenza virus proteins or fragments thereof. Exemplary influenza virus proteins that can be encoded by transgenes of second polynucleotides included in nucleic acid molecules provided herein include proteins from any human or animal virus, including influenza A virus, influenza B virus, influenza C virus, influenza D virus, or any combination thereof. Exemplary influenza proteins include hemagglutinin (HA), neuraminidase (NA), M2, M1, NP, NS1, NS2, PA, PB1, PB2, and PB1-F2. Hemagglutinin proteins from any influenza virus subtype, such as H1-H18 and any emerging hemagglutinin, and neuraminidase proteins from any influenza virus subtype, such as N1-N11 and any emerging neuraminidase, can be antigenic proteins encoded by transgenes included in second polynucleotides of nucleic acid molecules provided herein. Any suitable fragment of influenza virus proteins can be encoded by transgenes included in second polynucleotides of nucleic acid molecules provided herein, including, for example, one or more helper T lymphocyte (HTL) epitope, one or more cytotoxic T lymphocyte (CTL) epitope, or any combination thereof.

Transgenes included in second polynucleotides of nucleic acid molecules provided herein can express tumor proteins or tumor antigens. Tumor proteins or tumor antigens can be from any tumor, including solid and liquid tumors, for example. As used herein, the terms “tumor protein” or “tumor antigen,” which may be used interchangeably unless context clearly indicates otherwise, refer to any protein antigen that is present on the surface of a tumor cell or expressed in a tumor cell. Tumor proteins or tumor antigens include tumor-specific antigens that are present on tumor cells but generally not on other cells and tumor-associated antigens that are generally present on tumor cells and on normal cells. Tumor proteins or tumor antigens also include neoantigens. As used herein, the term “neoantigen” refers to tumor-specific mutations that can be unique to a patient's cancer or tumor.

As used herein, the term “tumor” refers to a mass or lump of tissue that is formed by an accumulation of abnormal cells. A tumor can be benign (i.e., not cancer), malignant (i.e., cancer), or premalignant (i.e., precancerous). The terms “tumor” and “neoplasm” can be used interchangeably. Generally, a cancerous tumor is malignant. As used herein, the term “solid tumor” refers to an abnormal mass of tissue that usually does not contain cysts or liquid areas. Exemplary solid tumors include sarcomas and carcinomas. As used herein, the term “liquid tumors” refers to tumors or cancers present in body fluids such as blood and bone marrow. Exemplary liquid tumors include hematopoietic tumors, such as leukemias and lymphomas, notwithstanding the ability of lymphomas to grow as solid tumors by growing in a lymph node, for example. The term “liquid tumor” can be used interchangeably with the term “blood cancer,” unless context clearly indicates otherwise.

Exemplary tumor proteins or tumor antigens include products of mutated oncogenes, products of mutated tumor suppressor genes, products of mutated genes other than oncogenes or tumor suppressors, tumor antigens produced by oncogenic viruses, altered cell surface glycoproteins, oncofetal antigens, and others. Tumor proteins or tumor antigens also include immune regulatory molecules, such as immune checkpoint inhibitors and immune stimulatory molecules. Tumor antigens further include altered cell surface glycolipids. In some aspects, the tumor protein or tumor antigen encoded by transgenes included in second polynucleotides of nucleic acid molecules provided herein is a kidney cancer, renal cancer, urinary bladder cancer, prostate cancer, uterine cancer, breast cancer, cervical cancer, ovarian cancer, lung cancer, liver cancer, stomach cancer, colon cancer, rectal cancer, oral cavity cancer, pharynx cancer, pancreatic cancer, thyroid cancer, melanoma, skin cancer, head and neck cancer, brain cancer, hematopoietic cancer, leukemia, lymphoma, bone cancer, or sarcoma protein. Exemplary tumor proteins or tumor antigens include KRAS, NRAS, HRAS, HER2, BRCA1, BRCA2, carcinoembryonic antigen (CEA), MUC1, guanylyl-cyclase C, NY-ESO-1, melanoma-associated antigen (e.g., MAGE-1, MAGE-3), p53, survivin, alphafetoprotein (AFP), CA-125, epithelial tumor antigen (ETA), tyrosinase, prostate-specific antigen (PSA), prostate-specific membrane antigen (PSMA), prostate stem cell antigen (PSCA), human aspartyl (asparaginyl) β-hydroxylase (HAAH), EphA2, and others.

In some aspects, the tumor protein or tumor antigen encoded by transgenes included in nucleic acid molecules provided herein is a wild-type protein or a fragment or epitope thereof. In other aspects, the tumor protein or tumor antigen encoded by transgenes included in second polynucleotides of nucleic acid molecules provided herein is a mutant protein or a fragment or epitope thereof. In one aspect, the tumor protein or tumor antigen is KRAS, NRAS, HRAS, HER2, BRCA1, BRCA2, carcinoembryonic antigen (CEA), MUC1, guanylyl-cyclase C, NY-ESO-1, melanoma-associated antigen (e.g., MAGE-1, MAGE-3), p53, survivin, alphafetoprotein (AFP), CA-125, epithelial tumor antigen (ETA), tyrosinase, prostate-specific antigen (PSA), prostate-specific membrane antigen (PSMA), prostate stem cell antigen (PSCA), human aspartyl (asparaginyl) β-hydroxylase (HAAH), EphA2, or any mutant thereof. In another aspect, the tumor protein or tumor antigen is KRAS or a fragment or epitope thereof. In another aspect, the tumor protein or tumor antigen is KRASG12D, KRASG12C, KRASG12V, or KRASG13D. Any KRAS that includes any mutation can be encoded by transgenes included in second polynucleotides of nucleic acid molecules provided herein.

In some aspects, transgenes included in second polynucleotides of nucleic acid molecules provided herein encode a reporter or a marker, including selectable markers. Reporters and markers can include fluorescent proteins, such as green fluorescent protein (GFP), red fluorescent protein (RFP), yellow fluorescent protein (YFP), luciferase enzymes, such as firefly and Renilla luciferases, and antibiotic selection markers, for example.

In some aspects, the second polynucleotide of nucleic acid molecules provided herein comprises at least two transgenes. Any number of transgenes can be included in second polynucleotides of nucleic acid molecules provided herein, such as one, two, three, four, five, six, seven, eight, nine, ten, or more transgenes. In one aspect, the second polynucleotide of nucleic acid molecules provided herein includes a second transgene encoding a second antigenic protein or a fragment thereof or an immunomodulatory protein. In one aspect, the second polynucleotide further comprises an internal ribosomal entry site (IRES), a sequence encoding a 2A peptide, or a combination thereof, located between transgenes. As used herein, the term “2A peptide” refers to a small (generally 18-22 amino acids) sequence that allows for efficient, stoichiometric production of discrete protein products within a single reading frame through a ribosomal skipping event within the 2A peptide sequence. As used herein, the term “internal ribosomal entry site” or “IRES” refers to a nucleotide sequence that allows for the initiation of protein translation of a messenger RNA (mRNA) sequence in the absence of an AUG start codon or without using an AUG start codon. An IRES can be found anywhere in an mRNA sequence, such as at or near the beginning, at or near the middle, or at or near the end of the mRNA sequence, for example.

Any number of transgenes included in second polynucleotides of nucleic acid molecules provided herein can be expressed via any combination of 2A peptide and IRES sequences. For example, a second transgene located 3′ of a first transgene can be expressed via a 2A peptide sequence or via an IRES sequence. As another example, a second transgene located 3′ of a first transgene and a third transgene located 3′ of the second transgene can be expressed via 2A peptide sequences located between the first and second transgenes and the second and third transgenes, via an IRES sequence located between the first and second transgenes and the second and third transgenes, via a 2A peptide sequence located between the first and second transgenes and an IRES located between the second and third transgenes, or via an IRES sequence located between the first and second transgenes and a 2A peptide sequence located between the second and third transgenes. Similar configurations and combinations of 2A peptide and IRES sequences located between transgenes are contemplated for any number of transgenes included in second polynucleotides of nucleic acid molecules provided herein. In addition to expression via 2A peptide and IRES sequences, two or more transgenes included in nucleic acid molecules provided herein can also be expressed from separate subgenomic RNAs.

A second, third, fourth, fifth, sixth, seventh, eighth, ninth, tenth, etc., transgene included in second polynucleotides of nucleic acid molecules provided herein can encode an immunomodulatory protein or a functional fragment or functional variant thereof. Any immunomodulatory protein or a functional fragment or functional variant thereof can be encoded by a transgene included in second polynucleotides.

As used herein, the terms “functional variant” or “functional fragment” refer to a molecule, including a nucleic acid or protein, for example, that comprises a nucleotide and/or amino acid sequence that is altered by one or more nucleotides and/or amino acids compared to the nucleotide and/or amino acid sequences of the parent or reference molecule. For a protein, a functional variant is still able to function in a manner that is similar to the parent molecule. In other words, the modifications in the amino acid and/or nucleotide sequence of the parent molecule do not significantly affect or alter the functional characteristics of the molecule encoded by the nucleotide sequence or containing the amino acid sequence. The functional variant may have conservative sequence modifications including nucleotide and amino acid substitutions, additions and deletions. These modifications can be introduced by standard techniques known in the art, such as site-directed mutagenesis and random PCR-mediated mutagenesis. Functional variants can also include, but are not limited to, derivatives that are substantially similar in primary structural sequence, but which contain, e.g., in vitro or in vivo modifications, chemical and/or biochemical, that are not found in the parent molecule. Such modifications include, inter alia, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI-anchor formation, hydroxylation, iodination, methylation, myristoylation, oxidation, pegylation, proteolytic processing, phosphorylation, prenylation, racemization, selenoylation, sulfation, transfer-RNA-mediated addition of amino acids to proteins such as arginylation, ubiquitination, and the like.

In one aspect, a second transgene included in second polynucleotides of nucleic acid molecules provided herein encodes a cytokine, a chemokine, or an interleukin. Exemplary cytokines include interferons, TNF-α, TGF-β, G-CSF, and GM-CSF. Exemplary chemokines include CCL3, CCL26, and CXCL7. Exemplary interleukins include IL-I, IL-2, IL-3, IL-4, IL-5, IL-6, IL-7, IL-8, IL-10, IL-12, IL-15, IL-18, IL-21, and IL-23. Any transgene or combination of transgenes encoding any cytokine, chemokine, interleukin, or combinations thereof, can be included in second polynucleotides of nucleic acid molecules provided herein.

In one aspect, first and second transgenes included in second polynucleotides of nucleic acid molecules provided herein encode viral proteins, bacterial proteins, fungal proteins, protozoan proteins, parasite proteins, tumor proteins, immunomodulatory proteins, or any combination thereof. In yet another aspect, first, second, third, fourth, fifth, sixth, seventh, eighth, ninth, tenth, or more transgenes included in second polynucleotides of nucleic acid molecules provided herein encode viral proteins, bacterial proteins, fungal proteins, protozoan proteins, parasite proteins, tumor proteins, immunomodulatory proteins, or any combination thereof.

DNA and RNA Molecules

Nucleic acid molecules provided herein can be DNA molecules or RNA molecules. It will be appreciated that T present in DNA is substituted with U in RNA, and vice versa. In one aspect, nucleic acid molecules provided herein are DNA molecules. In another aspect, DNA molecules provided herein further comprise a promoter. As used herein, the term “promoter” refers to a regulatory sequence that initiates transcription. A promoter can be operably linked to first and second polynucleotides of nucleic acid molecules provided herein. Generally, promoters included in DNA molecules provided herein include promoters for in vitro transcription (IVT). Any suitable promoter for in vitro transcription can be included in DNA molecules provided herein, such as a T7 promoter, a T3 promoter, an SP6 promoter, and others. In one aspect, DNA molecules provided herein comprise a T7 promoter. In another aspect, the promoter is located 5′ of the 5′ UTR included in DNA molecules provided herein. In yet another aspect, the promoter is a T7 promoter located 5′ of the 5′ UTR included in DNA molecules provided herein. In yet another aspect, the promoter overlaps with the 5′ UTR. A promoter and a 5′ UTR can overlap by about one nucleotide, about two nucleotides, about three nucleotides, about four nucleotides, about five nucleotides, about six nucleotides, about seven nucleotides, about eight nucleotides, about nine nucleotides, about ten nucleotides, about 11 nucleotides, about 12 nucleotides, about 13 nucleotides, about 14 nucleotides, about 15 nucleotides, about 16 nucleotides, about 17 nucleotides, about 18 nucleotides, about 19 nucleotides, about 20 nucleotides, about 21 nucleotides, about 22 nucleotides, about 23 nucleotides, about 24 nucleotides, about 25 nucleotides, about 26 nucleotides, about 27 nucleotides, about 28 nucleotides, about 29 nucleotides, about 30 nucleotides, about 31 nucleotides, about 32 nucleotides, about 33 nucleotides, about 34 nucleotides, about 35 nucleotides, about 36 nucleotides, about 37 nucleotides, about 38 nucleotides, about 39 nucleotides, about 40 nucleotides, about 41 nucleotides, about 42 nucleotides, about 43 nucleotides, about 44 nucleotides, about 45 nucleotides, about 46 nucleotides, about 47 nucleotides, about 48 nucleotides, about 49 nucleotides, about 50 nucleotides, or more nucleotides.

In some aspects, DNA molecules provided herein include a promoter for in vivo transcription. Generally, the promoter for in vivo transcription is an RNA polymerase II (RNA pol II) promoter. Any RNA pol II promoter can be included in DNA molecules provided herein, including constitutive promoters, inducible promoters, and tissue-specific promoters. Exemplary constitutive promoters include a cytomegalovirus (CMV) promoter, an EF1α promoter, an SV40 promoter, a PGK1 promoter, a Ubc promoter, a human beta actin promoter, a CAG promoter, and others. Any tissue-specific promoter can be included in DNA molecules provided herein. In one aspect, the RNA pol II promoter is a muscle-specific promoter, skin-specific promoter, subcutaneous tissue-specific promoter, liver-specific promoter, spleen-specific promoter, lymph node-specific promoter, or a promoter with any other tissue specificity. DNA molecules provided herein can also include an enhancer. Any enhancer that increases transcription can be included in DNA molecules provided herein.

In some aspects, nucleic acid molecules provided herein are RNA molecules. An RNA molecule provided herein can be generated by in vitro transcription (IVT) of DNA molecules provided herein. In one aspect, RNA molecules provided herein are self-replicating RNA molecules. In another aspect, RNA molecules provided herein further comprise a 5′ cap. Any 5′ cap can be included in RNA molecules provided herein, including 5′ caps having a Cap 1 structure, a Cap 1 (m6A) structure, a Cap 2 structure, a Cap 0 structure, or any combination thereof. In one aspect, RNA molecules provided herein include a 5′ cap having Cap 1 structure. In yet another aspect, RNA molecules provided herein are self-replicating RNA molecules comprising a 5′ cap having a Cap 1 structure. In a further aspect, RNA molecules provided herein comprise a cap having a Cap 1 structure, wherein a m7G is linked via a 5′-5′ triphosphate to the 5′ end of the 5′ UTR. In yet a further aspect, RNA molecules provided herein comprise a cap having a Cap 1 structure, wherein a m7G is linked via a 5′-5′ triphosphate to the 5′ end of the 5′ UTR comprising a sequence of SEQ ID NO:73. Any method of capping can be used, including, but not limited to using a Vaccinia Capping enzyme (New England Biolabs, Ipswich, Mass.) and co-transcriptional capping or capping at or shortly after initiation of in vitro transcription (IVT), by for example, including a capping agent as part of an in vitro transcription (IVT) reaction. (Nuc. Acids Symp. (2009) 53:129).

Provided herein, in some embodiments, are nucleic acid molecules comprising (a) a sequence of SEQ ID NO:78; or (b) a sequence of SEQ ID NO:72, SEQ ID NO:73, SEQ ID NO:76, and SEQ ID NO:77, wherein T is substituted with U. In one aspect, nucleic acid molecules provided herein are RNA molecules. In another aspect, RNA molecules provided herein further comprise a 5′ cap having a Cap 1 structure. Any RNA molecules provided herein can be self-replicating RNA molecules.

Only those mRNAs that carry the Cap structure are active in Cap dependent translation; “decapitation” of mRNA results in an almost complete loss of their template activity for protein synthesis (Nature, 255:33-37, (1975); J. Biol. Chem., vol. 253:5228-5231, (1978); and Proc. Natl. Acad. Sci. USA, 72:1189-1193, (1975)).

Another element of eukaryotic mRNA is the presence of 2′-O-methyl nucleoside residues at transcript position 1 (Cap 1), and in some cases, at transcript positions 1 and 2 (Cap 2). The 2′-O-methylation of mRNA provides higher efficacy of mRNA translation in vivo (Proc. Natl. Acad. Sci. USA, 77:3952-3956 (1980)) and further improves nuclease stability of the 5′-capped mRNA. The mRNA with Cap 1 (and Cap 2) is a distinctive mark that allows cells to recognize the bona fide mRNA 5′ end, and in some instances, to discriminate against transcripts emanating from infectious genetic elements (Nucleic Acid Research 43: 482-492 (2015)).

Some examples of 5′ cap structures and methods for preparing mRNAs comprising the same are given in WO2015/051169A2, WO/2015/061491, US 2018/0273576, and U.S. Pat. Nos. 8,093,367, 8,304,529, and 10,487,105. In some embodiments, the 5′ cap is m7GpppAmpG, which is known in the art. In some embodiments, the 5′ cap is m7GpppG or m7GpppGm, which are known in the art. Structural formulas for embodiments of 5′ cap structures are provided below.

In some embodiments, a self-replicating RNA of the disclosure comprises a 5′ cap having the structure of Formula (Cap I).

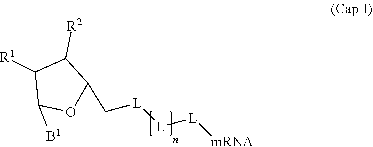

wherein B¹ is a natural or modified nucleobase; R¹ and R² are each independently selected from a halogen, OH, and OCH₃; each L is independently selected from the group consisting of phosphate, phophorothioate, and boranophosphate wherein each L is linked by diester bonds; n is 0 or 1. and mRNA represents an mRNA of the present disclosure linked at its 5′ end. In some embodiments B¹ is G, m⁷G, or A. In some embodiments, n is 0. In some embodiments n is 1. In some embodiments, B¹ is A or m⁶A and R¹ is OCH₃; wherein G is guanine, m⁷G is 7-methylguanine, A is adenine, and m⁶A is N⁶-methyladenine.

In some embodiments, a self-replicating RNA of the disclosure comprises a 5′ cap having the structure of Formula (Cap II).

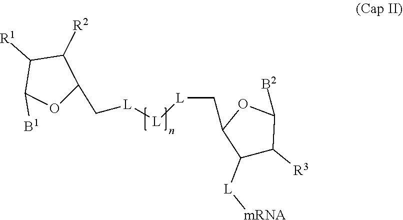

wherein B¹ and B² are each independently a natural or modified nucleobase; R¹, R², and R³ are each independently selected from a halogen, OH, and OCH₃, each L is independently selected from the group consisting of phosphate, phophorothioate, and boranophosphate wherein each L is linked by diester bonds; mRNA represents an mRNA of the present disclosure linked at its 5′ end; and n is 0 or 1. In some embodiments B¹ is G, m⁷G, or A. In some embodiments, n is 0. In some embodiments, n is 1. In some embodiments, B¹ is A or m⁶A and R¹ is OCH₃; wherein G is guanine, m⁷G is 7-methylguanine, A is adenine, and m⁶A is N⁶-methyladenine.

In some embodiments, a self-replicating RNA of the disclosure comprises a 5′ cap having the structure of Formula (Cap III).

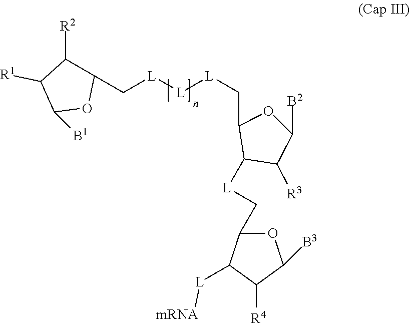

wherein B¹, B², and B³ are each independently a natural or modified nucleobase; R¹, R², R³, and R⁴ are each independently selected from a halogen, OH, and OCH₃; each L is independently selected from the group consisting of phosphate, phosphorothioate, and boranophosphate wherein each L is linked by diester bonds; mRNA represents an mRNA of the present disclosure linked at its 5′ end; and n is 0 or 1. In some embodiments, at least one of R¹, R², R³, and R⁴ is OH. In some embodiments B¹ is G, m⁷G, or A. In some embodiments, B¹ is A or m⁶A and R¹ is OCH₃; wherein G is guanine, m⁷G is 7-methylguanine, A is adenine, and m⁶A is N⁶-methyladenine. In some embodiments, n is 1.

In some embodiments, a self-replicating RNA of the disclosure comprises a m7GpppG 5′ cap analog having the structure of Formula (Cap IV).

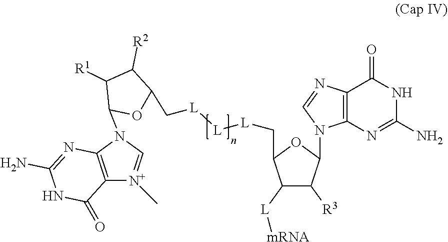

wherein, R¹, R², and R³ are each independently selected from a halogen, OH, and OCH₃, each L is independently selected from the group consisting of phosphate, phosphorothioate, and boranophosphate wherein each L is linked by diester bonds; mRNA represents an mRNA of the present disclosure linked at its 5′ end; n is 0 or 1. In some embodiments, at least one of R¹, R², and R³ is OH. In some embodiments, the 5′ cap is m⁷GpppG wherein R¹, R², and R³ are each OH, n is 1, and each L is a phosphate. In some embodiments, n is 1. In some embodiments, the 5′ cap is m7GpppGm, wherein R¹ and R² are each OH, R³ is OCH₃, each L is a phosphate, mRNA is the mRNA encoding an enzyme having OTC activity linked at its 5′ end, and n is 1.

In some embodiments, a self-replicating RNA of the disclosure comprises a m7Gpppm7G 5′ cap analog having the structure of Formula (Cap V).

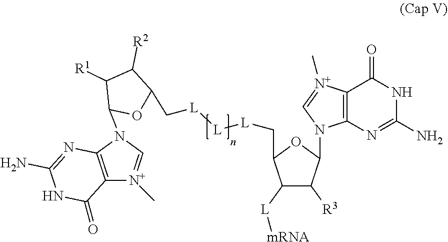

wherein, R¹, R², and R³ are each independently selected from a halogen, OH, and OCH₃, each L is independently selected from the group consisting of phosphate, phosphorothioate, and boranophosphate wherein each L is linked by diester bonds; mRNA represents an mRNA of the present disclosure linked at its 5′ end; and n is 0 or 1. In some embodiments, at least one of R¹, R², and R³ is OH. In some embodiments, n is 1.

In some embodiments, a self-replicating RNA of the disclosure comprises a m7Gpppm7GpN, 5′ cap analog, wherein N is a natural or modified nucleotide, the 5′ cap analog having the structure of Formula (Cap VI).

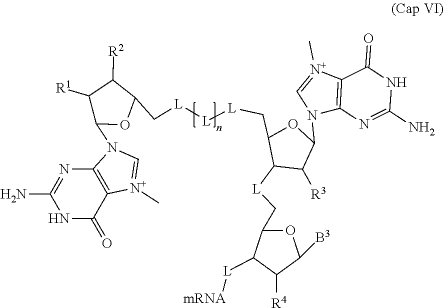

wherein B³ is a natural or modified nucleobase; R¹, R², R³, and R⁴ are each independently selected from a halogen, OH, and OCH₃, each L is independently selected from the group consisting of phosphate, phosphorothioate, and boranophosphate wherein each L is linked by diester bonds; mRNA represents an mRNA of the present disclosure linked at its 5′ end; and n is 0 or 3. In some embodiments, at least one of R¹, R², R³, and R⁴ is OH. In some embodiments B¹ is G, m⁷G, or A. In some embodiments, B¹ is A or m⁶A and R¹ is OCH₃; wherein G is guanine, m⁷G is 7-methylguanine, A is adenine, and m⁶A is N⁶-methyladenine. In some embodiments, n is 1.

In some embodiments, a self-replicating RNA of the disclosure comprises a m7Gpppm7GpG 5′ cap analog having the structure of Formula (Cap VII).

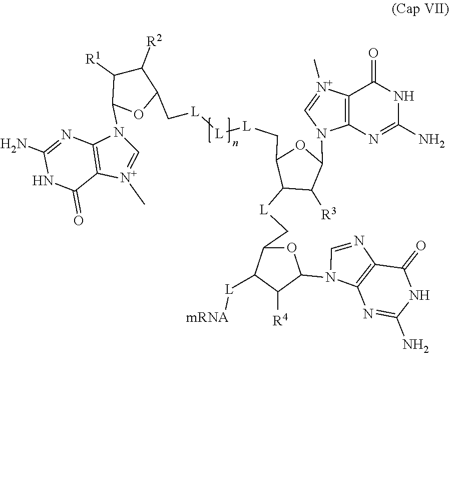

wherein, R¹, R², R³, and R⁴ are each independently selected from a halogen, OH, and OCH₃, each L is independently selected from the group consisting of phosphate, phosphorothioate, and boranophosphate wherein each L is linked by diester bonds; mRNA represents an mRNA of the present disclosure linked at its 5′ end; and n is 0 or 1. In some embodiments, at least one of R¹, R², R³, and R⁴ is OH. In some embodiments, n is 1.

In some embodiments, a self-replicating RNA of the disclosure comprises a m7Gpppm7Gpm7G 5′ cap analog having the structure of Formula (Cap VIII).

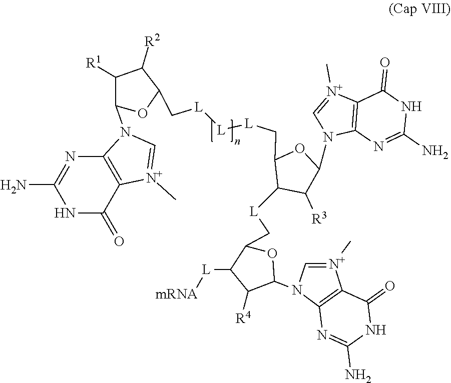

wherein, R¹, R², R³, and R⁴ are each independently selected from a halogen, OH, and OCH₃, each L is independently selected from the group consisting of phosphate, phosphorothioate, and boranophosphate wherein each L is linked by diester bonds; mRNA represents an mRNA of the present disclosure linked at its 5′ end; n is 0 or 1. In some embodiments, at least one of R¹, R², R³, and R⁴ is OH. In some embodiments, n is 1.

In some embodiments, a self-replicating RNA of the disclosure comprises a m7GpppA 5′ cap analog having the structure of Formula (Cap IX).

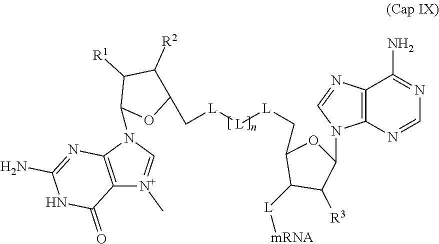

wherein, R¹, R², and R³ are each independently selected from a halogen, OH, and OCH₃, each L is independently selected from the group consisting of phosphate, phosphorothioate, and boranophosphate wherein each L is linked by diester bonds; mRNA represents an mRNA of the present disclosure linked at its 5′ end; and n is 0 or 1. In some embodiments, at least one of R¹, R², and R³ is OH. In some embodiments, n is 1.

In some embodiments, a self-replicating RNA of the disclosure comprises a m7GpppApN 5′ cap analog, wherein N is a natural or modified nucleotide, and the 5′ cap has the structure of Formula (Cap X).

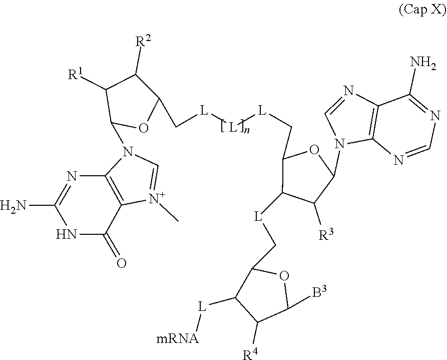

wherein B³ is a natural or modified nucleobase; R¹, R², R³, and R⁴ are each independently selected from a halogen, OH, and OCH₃, each L is independently selected from the group consisting of phosphate, phosphorothioate, and boranophosphate wherein each L is linked by diester bonds; mRNA represents an mRNA of the present disclosure linked at its 5′ end; and n is 0 or 1. In some embodiments, at least one of R¹, R², R³, and R⁴ is OH. In some embodiments B³ is G, m⁷G, A or m⁶A; wherein G is guanine, m⁷G is 7-methylguanine, A is adenine, and m⁶A is N⁶-methyladenine. In some embodiments, n is 1.

In some embodiments, a self-replicating RNA of the disclosure comprises a m7GpppAmpG 5′ cap analog having the structure of Formula (Cap XI).

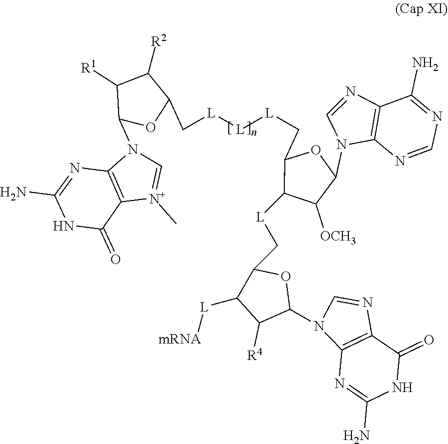

wherein, R¹, R², and R⁴ are each independently selected from a halogen, OH, and OCH₃, each L is independently selected from the group consisting of phosphate, phosphorothioate, and boranophosphate wherein each L is linked by diester bonds; mRNA represents an mRNA of the present disclosure linked at its 5′ end; and n is 0 or 1. In some embodiments, at least one of R¹, R², and R⁴ is OH. In some embodiments, the compound of Formula Cap XI is m⁷GpppAmpG, wherein R¹, R², and R⁴ are each OH, n is 1, and each L is a phosphate linkage. In some embodiments, n is 1.

In some embodiments, a self-replicating RNA of the disclosure comprises a m7GpppApm7G 5′ cap analog having the structure of Formula (Cap XII).

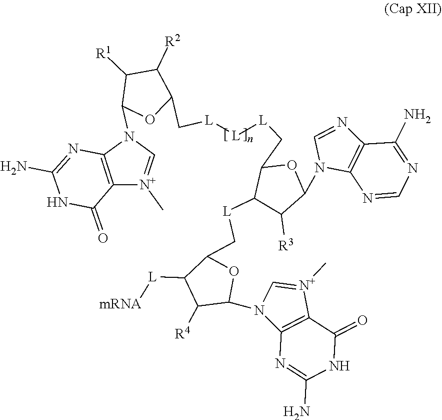

wherein, R¹, R², R³, and R⁴ are each independently selected from a halogen, OH, and OCH₃, each L is independently selected from the group consisting of phosphate, phosphorothioate, and boranophosphate wherein each L is linked by diester bonds; mRNA represents an mRNA of the present disclosure linked at its 5′ end; and n is 0 or 1. In some embodiments, at least one of R¹, R², R³, and R⁴ is OH. In some embodiments, n is 1.

In some embodiments, a self-replicating RNA of the disclosure comprises a m7GpppAmpm7G 5′ cap analog having the structure of Formula (Cap XIII).

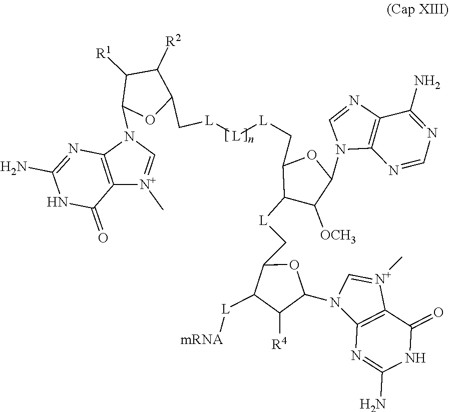

wherein, R¹, R², and R⁴ are each independently selected from a halogen, OH, and OCH₃, each L is independently selected from the group consisting of phosphate, phosphorothioate, and boranophosphate wherein each L is linked by diester bonds; mRNA represents an mRNA of the present disclosure linked at its 5′ end; and n is 0 or 1. In some embodiments, at least one of R¹, R², and R⁴ is OH. In some embodiments, n is 1.

Poly-Adenine (Poly-A) Tail

Polyadenylation is the addition of a poly(A) tail, a chain of adenine nucleotides usually about 100-120 monomers in length, to a mRNA. In eukaryotes, polyadenylation is part of the process that produces mature mRNA for translation and begins as the transcription of a gene terminates. The 3′-most segment of a newly made pre-mRNA is first cleaved off by a set of proteins; these proteins then synthesize the poly(A) tail at the 3′ end. The poly(A) tail is important for the nuclear export, translation, and stability of mRNA. The tail is shortened over time, and, when it is short enough, the mRNA is enzymatically degraded. However, in a few cell types, mRNAs with short poly(A) tails are stored for later activation by re-polyadenylation in the cytosol.

Preferably, a self-replicating RNA of the disclosure comprises a 3′ tail region, which can serve to protect the RNA from exonuclease degradation. The tail region may be a 3′poly(A) and/or 3′poly(C) region. Preferably, the tail region is a 3′ poly(A) tail. As used herein a “3′ poly(A) tail” is a polymer of sequential adenine nucleotides that can range in size from, for example: 10 to 250 sequential adenine nucleotides; 60-125 sequential adenine nucleotides, 90-125 sequential adenine nucleotides, 95-125 sequential adenine nucleotides, 95-121 sequential adenine nucleotides, 100 to 121 sequential adenine nucleotides, 110-121 sequential adenine nucleotides; 112-121 sequential adenine nucleotides; 114-121 adenine sequential nucleotides; or 115 to 121 sequential adenine nucleotides. Preferably, a 3′ poly(A) tail as described herein comprise 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, or 125 sequential adenine nucleotides. 3′ Poly(A) tails can be added using a variety of methods known in the art, e.g., using poly(A) polymerase to add tails to synthetic or in vitro transcribed RNA. Other methods include the use of a transcription vector to encode poly(A) tails or the use of a ligase (e.g., via splint ligation using a T4 RNA ligase and/or T4 DNA ligase), wherein poly(A) may be ligated to the 3′ end of a sense RNA. In some embodiments, a combination of any of the above methods is utilized.

Design and Synthesis of Self-Replicating RNA

The constructs for exemplary self-replicating RNA sequences of the present disclosure are provided in Table 4.

TABLE 4
Comparison of STARR^{TM} self-replicating RNA of the disclosure with
comparative self-replicating RNA as described

| | | Sequence | |
|---|---|---|---|
| Construct | Position | Type | Sequence |
| STARR^{TM} | 5' UTR | nucleotide | ATGGGCGGCGCATGAGAGAAGCCCAGACCAATTACCT |
| (SEQ ID NO: | | | ACCCAAA |
| 49) | | | |
| STARR^{TM} | non- | nucleotide | ATGGAGAAAGTTCACGTTGACATCGAGGAAGACAGCC |
| (SEQ ID NO: | structural | | CATTCCTCAGAGCTTTGCAGCGGAGCTTCCCGCAGTTT |
| 50) | gene ORF | | GAGGTAGAAGCCAAGCAGGTCACTGATAATGACCATG |
| | | | CTAATGCCAGAGCGTTTTCGCATCTGGCTTCAAAACTG |
| | | | ATCGAAACGGAGGTGGACCCATCCGACACGATCCTTG |
| | | | ACATTGGAAGTGCGCCCGCCCGCAGAATGTATTCTAA |
| | | | GCACAAGTATCATTGTATCTGTCCGATGAGATGTGCGG |
| | | | AAGATCCGGACAGATTGTATAAGTATGCAACTAAGCT |
| | | | GAAGAAAAACTGTAAGGAAATAACTGATAAGGAATTG |
| | | | GACAAGAAAATGAAGGAGCTGGCCGCCGTCATGAGCG |
| | | | ACCCTGACCTGGAAACTGAGACTATGTGCCTCCACGA |
| | | | CGACGAGTCGTGTCGCTACGAAGGGCAAGTCGCTGTT |
| | | | TACCAGGATGTATACGCCGTCGACGGCCCCACCAGCC |
| | | | TGTACCACCAGGCCAACAAGGGCGTGAGGGTGGCCTA |
| | | | CTGGATCGGCTTCGACACCACACCCTTCATGTTCAAGA |
| | | | ACCTGGCCGGCGCCTACCCCAGCTACAGCACCAACTG |
| | | | GGCCGACGAGACCGTGCTGACCGCCAGGAACATCGGC |
| | | | CTGTGCAGCAGCGACGTGATGGAGAGGAGCCGGAGAG |
| | | | GCATGAGCATCCTGAGGAAGAAATACCTGAAGCCCAG |
| | | | CAACAACGTGCTGTTCAGCGTGGGCAGCACCATCTAC |
| | | | CACGAGAAGAGGGACCTGCTCAGGAGCTGGCACCTGC |
| | | | CCAGCGTGTTCCACCTGAGGGGCAAGCAGAACTACAC |
| | | | CTGCAGGTGCGAGACCATCGTGAGCTGCGACGGCTAC |
| | | | GTGGTGAAGAGGATCGCCATCAGCCCCGGCCTGTACG |
| | | | GCAAGCCCAGCGGCTACGCCGCTACAATGCACAGGGA |
| | | | GGGCTTCCTGTGCTGCAAGGTGACCGACACCCTGAAC |
| | | | GGCGAGAGGGTGAGCTTCCCCGTGTGCACCTACGTGC |
| | | | CCGCCACCCTGTGCGACCAGATGACCGGCATCCTGGC |
| | | | CACCGACGTGAGCGCCGACGACGCCCAGAAGCTGCTC |
| | | | GTGGGCCTGAACCAGAGGATCGTGGTCAACGGCAGGA |
| | | | CCCAGAGGAACACCAACACAATGAAGAACTACCTGCT |
| | | | GCCCGTGGTGGCCCAGGCTTTCGCCAGGTGGGCCAAG |
| | | | GAGTACAAGGAGGACCAGGAAGACGAGAGGCCCCTG |
| | | | GGCCTGAGGGACAGGCAGCTGGTGATGGGCTGCTGCT |
| | | | GGGCCTTCAGGCGGCACAAGATCACCAGCATCTACAA |
| | | | GAGGCCCGACACCCAGACCATCATCAAGGTGAACAGC |
| | | | GACTTCCACAGCTTCGTGCTGCCCAGGATCGGCAGCA |
| | | | ACACCCTGGAGATCGGCCTGAGGACCCGGATCAGGAA |
| | | | GATGCTGGAGGAACACAAGGAGCCCAGCCCACTGATC |
| | | | ACCGCCGAGGACGTGCAGGAGGCCAAGTGCGCTGCCG |
| | | | ACGAGGCCAAGGAGGTGAGGGAGGCCGAGGAACTGA |
| | | | GGGCCGCCCTGCCACCCCTGGCTGCCGACGTGGAGGA |
| | | | ACCCACCCTGGAAGCCGACGTGGACCTGATGCTGCAG |
| | | | GAGGCCGGCGCCGGAAGCGTGGAGACACCCAGGGGC |
| | | | CTGATCAAGGTGACCAGCTACGACGGCGAGGACAAGA |
| | | | TCGGCAGCTACGCCGTGCTGAGCCCACAGGCCGTGCT |
| | | | GAAGTCCGAGAAGCTGAGCTGCATCCACCCACTGGCC |
| | | | GAGCAGGTGATCGTGATCACCCACAGCGGCAGGAAGG |
| | | | GCAGGTACGCCGTGGAGCCCTACCACGGCAAGGTGGT |
| | | | CGTGCCCGAGGGCCACGCCATCCCCGTGCAGGACTTC |
| | | | CAGGCCCTGAGCGAGAGCGCCACCATCGTGTACAACG |
| | | | AGAGGGAGTTCGTGAACAGGTACCTGCACCATATCGC |
| | | | CACCCACGGCGGAGCCCTGAACACCGACGAGGAATAC |
| | | | TACAAGACCGTGAAGCCCAGCGAGCACGACGGCGAGT |
| | | | ACCTGTACGACATCGACAGGAAGCAGTGCGTGAAGAA |
| | | | AGAGCTGGTGACCGGCCTGGGACTGACCGGCGAGCTG |
| | | | GTGGACCCACCCTTCCACGAGTTCGCCTACGAGAGCCT |
| | | | GAGGACCAGACCCGCCGCTCCCTACCAGGTGCCCACC |
| | | | ATCGGCGTGTACGGCGTGCCCGGCAGCGGAAAGAGCG |
| | | | GCATCATCAAGAGCGCCGTGACCAAGAAAGACCTGGT |
| | | | GGTCAGCGCCAAGAAAGAGAACTGCGCCGAGATCATC |
| | | | AGGGACGTGAAGAAGATGAAAGGCCTGGACGTGAAC |
| | | | GCGCGCACCGTGGACAGCGTGCTGCTGAACGGCTGCA |
| | | | AGCACCCCGTGGAGACCCTGTACATCGACGAGGCCTT |
| | | | CGCTTGCCACGCCGGCACCCTGAGGGCCCTGATCGCC |
| | | | ATCATCAGGCCCAAGAAAGCCGTGCTGTGCGGCGACC |
| | | | CCAAGCAGTGCGGCTTCTTCAACATGATGTGCCTGAAG |
| | | | GTGCACTTCAACCACGAGATCTGCACCCAGGTGTTCCA |
| | | | CAAGAGCATCAGCAGGCGGTGCACCAAGAGCGTGACC |
| | | | AGCGTCGTGAGCACCCTGTTCTACGACAAGAAAATGA |
| | | | GGACCACCAACCCCAAGGAGACCAAAATCGTGATCGA |
| | | | CACCACAGGCAGCACCAAGCCCAAGCAGGACGACCTG |
| | | | ATCCTGACCTGCTTCAGGGGCTGGGTGAAGCAGCTGC |
| | | | AGATCGACTACAAGGGCAACGAGATCATGACCGCCGC |
| | | | TGCCAGCCAGGGCCTGACCAGGAAGGGCGTGTACGCC |
| | | | GTGAGGTACAAGGTGAACGAGAACCCACTGTACGCTC |
| | | | CCACCAGCGAGCACGTGAACGTGCTGCTGACCAGGAC |
| | | | CGAGGACAGGATCGTGTGGAAGACCCTGGCCGGCGAC |
| | | | CCCTGGATCAAGACCCTGACCGCCAAGTACCCCGGCA |
| | | | ACTTCACCGCCACCATCGAAGAGTGGCAGGCCGAGCA |
| | | | CGACGCCATCATGAGGCACATCCTGGAGAGGCCCGAC |
| | | | CCCACCGACGTGTTCCAGAACAAGGCCAACGTGTGCT |
| | | | GGGCCAAGGCCCTGGTGCCCGTGCTGAAGACCGCCGG |
| | | | CATCGACATGACCACAGAGCAGTGGAACACCGTGGAC |
| | | | TACTTCGAGACCGACAAGGCCCACAGCGCCGAGATCG |
| | | | TGCTGAACCAGCTGTGCGTGAGGTTCTTCGGCCTGGAC |
| | | | CTGGACAGCGGCCTGTTCAGCGCCCCCACCGTGCCACT |
| | | | GAGCATCAGGAACAACCACTGGGACAACAGCCCCAGC |
| | | | CCAAACATGTACGGCCTGAACAAGGAGGTGGTCAGGC |
| | | | AGCTGAGCAGGCGGTACCCACAGCTGCCCAGGGCCGT |
| | | | GGCCACCGGCAGGGTGTACGACATGAACACCGGCACC |
| | | | CTGAGGAACTACGACCCCAGGATCAACCTGGTGCCCG |
| | | | TGAACAGGCGGCTGCCCCACGCCCTGGTGCTGCACCA |
| | | | CAACGAGCACCCACAGAGCGACTTCAGCTCCTTCGTG |
| | | | AGCAAGCTGAAAGGCAGGACCGTGCTGGTCGTGGGCG |
| | | | AGAAGCTGAGCGTGCCCGGCAAGATGGTGGACTGGCT |
| | | | GAGCGACAGGCCCGAGGCCACCTTCCGGGCCAGGCTG |
| | | | GACCTCGGCATCCCCGGCGACGTGCCCAAGTACGACA |
| | | | TCATCTTCGTGAACGTCAGGACCCCATACAAGTACCAC |
| | | | CATTACCAGCAGTGCGAGGACCACGCCATCAAGCTGA |
| | | | GCATGCTGACCAAGAAGGCCTGCCTGCACCTGAACCC |
| | | | CGGAGGCACCTGCGTGAGCATCGGCTACGGCTACGCC |
| | | | GACAGGGCCAGCGAGAGCATCATTGGCGCCATCGCCA |
| | | | GGCTGTTCAAGTTCAGCAGGGTGTGCAAACCCAAGAG |
| | | | CAGCCTGGAGGAAACCGAGGTGCTGTTCGTGTTCATC |
| | | | GGCTACGACCGGAAGGCCAGGACCCACAACCCCTACA |
| | | | AGCTGAGCAGCACCCTGACAAACATCTACACCGGCAG |
| | | | CAGGCTGCACGAGGCCGGCTGCGCCCCCAGCTACCAC |
| | | | GTGGTCAGGGGCGATATCGCCACCGCCACCGAGGGCG |
| | | | TGATCATCAACGCTGCCAACAGCAAGGGCCAGCCCGG |
| | | | AGGCGGAGTGTGCGGCGCCCTGTACAAGAAGTTCCCC |
| | | | GAGAGCTTCGACCTGCAGCCCATCGAGGTGGGCAAGG |
| | | | CCAGGCTGGTGAAGGGCGCCGCTAAGCACATCATCCA |
| | | | CGCCGTGGGCCCCAACTTCAACAAGGTGAGCGAGGTG |
| | | | GAAGGCGACAAGCAGCTGGCCGAAGCCTACGAGAGC |
| | | | ATCGCCAAGATCGTGAACGACAATAACTACAAGAGCG |
| | | | TGGCCATCCCACTGCTCAGCACCGGCATCTTCAGCGGC |
| | | | AACAAGGACAGGCTGACCCAGAGCCTGAACCACCTGC |
| | | | TCACCGCCCTGGACACCACCGATGCCGACGTGGCCAT |
| | | | CTACTGCAGGGACAAGAAGTGGGAGATGACCCTGAAG |
| | | | GAGGCCGTGGCCAGGCGGGAGGCCGTGGAAGAGATCT |
| | | | GCATCAGCGACGACTCCAGCGTGACCGAGCCCGACGC |
| | | | CGAGCTGGTGAGGGTGCACCCCAAGAGCTCCCTGGCC |
| | | | GGCAGGAAGGGCTACAGCACCAGCGACGGCAAGACCT |
| | | | TCAGCTACCTGGAGGGCACCAAGTTCCACCAGGCCGC |
| | | | TAAGGACATCGCCGAGATCAACGCTATGTGGCCCGTG |
| | | | GCCACCGAGGCCAACGAGCAGGTGTGCATGTACATCC |
| | | | TGGGCGAGAGCATGTCCAGCATCAGGAGCAAGTGCCC |
| | | | CGTGGAGGAAAGCGAGGCCAGCACACCACCCAGCACC |
| | | | CTGCCCTGCCTGTGCATCCACGCTATGACACCCGAGAG |
| | | | GGTGCAGCGGCTGAAGGCCAGCAGGCCCGAGCAGATC |
| | | | ACCGTGTGCAGCTCCTTCCCACTGCCCAAGTACAGGAT |
| | | | CACCGGCGTGCAGAAGATCCAGTGCAGCCAGCCCATC |
| | | | CTGTTCAGCCCAAAGGTGCCCGCCTACATCCACCCCAG |
| | | | GAAGTACCTGGTGGAGACCCCACCCGTGGACGAGACA |
| | | | CCCGAGCCAAGCGCCGAGAACCAGAGCACCGAGGGC |
| | | | ACACCCGAGCAGCCACCCCTGATCACCGAGGACGAGA |
| | | | CAAGGACCCGGACCCCAGAGCCCATCATTATCGAGGA |
| | | | AGAGGAAGAGGACAGCATCAGCCTGCTGAGCGACGGC |
| | | | CCCACCCACCAGGTGCTGCAGGTGGAGGCCGACATCC |
| | | | ACGGCCCACCCAGCGTGTCCAGCTCCAGCTGGAGCAT |
| | | | CCCACACGCCAGCGACTTCGACGTGGACAGCCTGAGC |
| | | | ATCCTGGACACCCTGGAGGGCGCCAGCGTGACCTCCG |
| | | | GCGCCACCAGCGCCGAGACCAACAGCTACTTCGCCAA |
| | | | GAGCATGGAGTTCCTGGCCAGGCCCGTGCCAGCTCCC |
| | | | AGGACCGTGTTCAGGAACCCACCCCACCCAGCTCCCA |
| | | | GGACCAGGACCCCAAGCCTGGCTCCCAGCAGGGCCTG |
| | | | CAGCAGGACCAGCCTGGTGAGCACCCCACCCGGCGTG |
| | | | AACAGGGTGATCACCAGGGAGGAACTGGAGGCCCTGA |
| | | | CACCCAGCAGGACCCCCAGCAGGTCCGTGAGCAGGAC |
| | | | TAGTCTGGTGTCCAACCCACCCGGCGTGAACAGGGTG |
| | | | ATCACCAGGGAGGAATTCGAGGCCTTCGTGGCCCAGC |
| | | | AACAGAGACGGTTCGACGCCGGCGCCTACATCTTCAG |
| | | | CAGCGACACCGGCCAGGGACACCTGCAGCAAAAGAGC |
| | | | GTGAGGCAGACCGTGCTGAGCGAGGTGGTGCTGGAGA |
| | | | GGACCGAGCTGGAAATCAGCTACGCCCCCAGGCTGGA |
| | | | CCAGGAGAAGGAGGAACTGCTCAGGAAGAAACTGCA |
| | | | GCTGAACCCCACCCCAGCCAACAGGAGCAGGTACCAG |
| | | | AGCAGGAAGGTGGAGAACATGAAGGCCATCACCGCCA |
| | | | GGCGGATCCTGCAGGGCCTGGGACACTACCTGAAGGC |
| | | | CGAGGGCAAGGTGGAGTGCTACAGGACCCTGCACCCC |
| | | | GTGCCACTGTACAGCTCCAGCGTGAACAGGGCCTTCTC |
| | | | CAGCCCCAAGGTGGCCGTGGAGGCCTGCAACGCTATG |
| | | | CTGAAGGAGAACTTCCCCACCGTGGCCAGCTACTGCA |
| | | | TCATCCCCGAGTACGACGCCTACCTGGACATGGTGGA |
| | | | CGGCGCCAGCTGCTGCCTGGACACCGCCAGCTTCTGCC |
| | | | CCGCCAAGCTGAGGAGCTTCCCCAAGAAACACAGCTA |
| | | | CCTGGAGCCCACCATCAGGAGCGCCGTGCCCAGCGCC |
| | | | ATCCAGAACACCCTGCAGAACGTGCTGGCCGCTGCCA |
| | | | CCAAGAGGAACTGCAACGTGACCCAGATGAGGGAGCT |
| | | | GCCCGTGCTGGACAGCGCTGCCTTCAACGTGGAGTGCT |
| | | | TCAAGAAATACGCCTGCAACAACGAGTACTGGGAGAC |
| | | | CTTCAAGGAGAACCCCATCAGGCTGACCGAAGAGAAC |
| | | | GTGGTGAACTACATCACCAAGCTGAAGGGCCCCAAGG |
| | | | CCGCTGCCCTGTTCGCTAAGACCCACAACCTGAACATG |
| | | | CTGCAGGACATCCCAATGGACAGGTTCGTGATGGACC |
| | | | TGAAGAGGGACGTGAAGGTGACACCCGGCACCAAGCA |
| | | | CACCGAGGAGAGGCCCAAGGTGCAGGTGATCCAGGCC |
| | | | GCTGACCCACTGGCCACCGCCTACCTGTGCGGCATCCA |
| | | | CAGGGAGCTGGTGAGGCGGCTGAACGCCGTGCTGCTG |
| | | | CCCAACATCCACACCCTGTTCGACATGAGCGCCGAGG |
| | | | ACTTCGACGCCATCATCGCCGAGCACTTCCAGCCCGGC |
| | | | GACTGCGTGCTGGAGACCGACATCGCCAGCTTCGACA |
| | | | AGAGCGAGGATGACGCTATGGCCCTGACCGCTCTGAT |
| | | | GATCCTGGAGGACCTGGGCGTGGACGCCGAGCTGCTC |
| | | | ACCCTGATCGAGGCTGCCTTCGGCGAGATCAGCTCCAT |
| | | | CCACCTGCCCACCAAGACCAAGTTCAAGTTCGGCGCT |
| | | | ATGATGAAAAGCGGAATGTTCCTGACCCTGTTCGTGA |
| | | | ACACCGTGATCAACATTGTGATCGCCAGCAGGGTGCT |
| | | | GCGGGAGAGGCTGACCGGCAGCCCCTGCGCTGCCTTC |
| | | | ATCGGCGACGACAACATCGTGAAGGGCGTGAAAAGCG |
| | | | ACAAGCTGATGGCCGACAGGTGCGCCACCTGGCTGAA |
| | | | CATGGAGGTGAAGATCATCGACGCCGTGGTGGGCGAG |
| | | | AAGGCCCCCTACTTCTGCGGCGGATTCATCCTGTGCGA |
| | | | CAGCGTGACCGGCACCGCCTGCAGGGTGGCCGACCCC |
| | | | CTGAAGAGGCTGTTCAAGCTGGGCAAGCCACTGGCCG |
| | | | CTGACGATGAGCACGACGATGACAGGCGGAGGGCCCT |
| | | | GCACGAGGAAAGCACCAGGTGGAACAGGGTGGGCAT |
| | | | CCTGAGCGAGCTGTGCAAGGCCGTGGAGAGCAGGTAC |
| | | | GAGACCGTGGGCACCAGCATCATCGTGATGGCTATGA |
| | | | CCACACTGGCCAGCTCCGTCAAGAGCTTCTCCTACCTG |
| | | | AGGGGGGCCCCTATAACTCTCTACGGCTAA |
| STARR^{TM} | non- | amino acid | MEKVHVDIEEDSPFLRALQRSFPQFEVEAKQVTDNDHAN |
| (SEQ ID NO: | structural | | ARAFSHLASKLIETEVDPSDTILDIGSAPARRMYSKHKYH |
| 51) | gene ORF | | CICPMRCAEDPDRLYKYATKLKKNCKEITDKELDKKMK |
| | | | ELAAVMSDPDLETETMCLHDDESCRYEGQVAVYQDVY |
| | | | AVDGPTSLYHQANKGVRVAYWIGFDTTPFMFKNLAGAY |
| | | | PSYSTNWADETVLTARNIGLCSSDVMERSRRGMSILRKK |
| | | | YLKPSNNVLFSVGSTIYHEKRDLLRSWHLPSVFHLRGKQ |
| | | | NYTCRCETIVSCDGYVVKRIAISPGLYGKPSGYAATMHR |
| | | | EGFLCCKVTDTLNGERVSFPVCTYVPATLCDQMTGILAT |
| | | | DVSADDAQKLLVGLNQRIVVNGRTQRNTNTMKNYLLPV |
| | | | VAQAFARWAKEYKEDQEDERPLGLRDRQLVMGCCWAF |
| | | | RRHKITSIYKRPDTQTIIKVNSDFHSFVLPRIGSNTLEIGLR |
| | | | TRIRKMLEEHKEPSPLITAEDVQEAKCAADEAKEVREAE |
| | | | ELRAALPPLAADVEEPTLEADVDLMLQEAGAGSVETPRG |
| | | | LIKVTSYDGEDKIGSYAVLSPQAVLKSEKLSCIHPLAEQVI |
| | | | VITHSGRKGRYAVEPYHGKVVVPEGHAIPVQDFQALSES |
| | | | ATIVYNEREFVNRYLHHIATHGGALNTDEEYYKTVKPSE |
| | | | HDGEYLYDIDRKQCVKKELVTGLGLTGELVDPPFHEFAY |
| | | | ESLRTRPAAPYQVPTIGVYGVPGSGKSGIIKSAVTKKDLV |
| | | | VSAKKENCAEIIRDVKKMKGLDVNARTVDSVLLNGCKH |
| | | | PVETLYIDEAFACHAGTLRALIAIIRPKKAVLCGDPKQCG |
| | | | FFNMMCLKVHFNHEICTQVFHKSISRRCTKSVTSVVSTLF |
| | | | YDKKMRTTNPKETKIVIDTTGSTKPKQDDLILTCFRGWV |
| | | | KQLQIDYKGNEIMTAAASQGLTRKGVYAVRYKVNENPL |
| | | | YAPTSEHVNVLLTRTEDRIVWKTLAGDPWIKTLTAKYPG |
| | | | NFTATIEEWQAEHDAIMRHILERPDPTDVFQNKANVCWA |
| | | | KALVPVLKTAGIDMTTEQWNTVDYFETDKAHSAEIVLN |
| | | | QLCVRFFGLDLDSGLFSAPTVPLSIRNNHWDNSPSPNMY |
| | | | GLNKEVVRQLSRRYPQLPRAVATGRVYDMNTGTLRNYD |
| | | | PRINLVPVNRRLPHALVLHHNEHPQSDFSSFVSKLKGRTV |
| | | | LVVGEKLSVPGKMVDWLSDRPEATFRARLDLGIPGDVP |
| | | | KYDIIFVNVRTPYKYHHYQQCEDHAIKLSMLTKKACLHL |
| | | | NPGGTCVSIGYGYADRASESIIGAIARLFKFSRVCKPKSSL |
| | | | EETEVLFVFIGYDRKARTHNPYKLSSTLTNIYTGSRLHEA |
| | | | GCAPSYHVVRGDIATATEGVIINAANSKGQPGGGVCGAL |
| | | | YKKFPESFDLQPIEVGKARLVKGAAKHIIHAVGPNFNKVS |
| | | | EVEGDKQLAEAYESIAKIVNDNNYKSVAIPLLSTGIFSGN |
| | | | KDRLTQSLNHLLTALDTTDADVAIYCRDKKWEMTLKEA |
| | | | VARREAVEEICISDDSSVTEPDAELVRVHPKSSLAGRKGY |
| | | | STSDGKTFSYLEGTKFHQAAKDIAEINAMWPVATEANEQ |
| | | | VCMYILGESMSSIRSKCPVEESEASTPPSTLPCLCIHAMTP |
| | | | ERVQRLKASRPEQITVCSSFPLPKYRITGVQKIQCSQPILFS |
| | | | PKVPAYIHPRKYLVETPPVDETPEPSAENQSTEGTPEQPPL |
| | | | ITEDETRTRTPEPIIIEEEEEDSISLLSDGPTHQVLQVEADIH |
| | | | GPPSVSSSSWSIPHASDFDVDSLSILDTLEGASVTSGATSA |
| | | | ETNSYFAKSMEFLARPVPAPRTVFRNPPHPAPRTRTPSLA |
| | | | PSRACSRTSLVSTPPGVNRVITREELEALTPSRTPSRSVSR |
| | | | TSLVSNPPGVNRVITREEFEAFVAQQQRRFDAGAYIFSSD |
| | | | TGQGHLQQKSVRQTVLSEVVLERTELEISYAPRLDQEKE |
| | | | ELLRKKLQLNPTPANRSRYQSRKVENMKAITARRILQGL |
| | | | GHYLKAEGKVECYRTLHPVPLYSSSVNRAFSSPKVAVEA |
| | | | CNAMLKENFPTVASYCIIPEYDAYLDMVDGASCCLDTAS |
| | | | FCPAKLRSFPKKHSYLEPTIRSAVPSAIQNTLQNVLAAAT |
| | | | KRNCNVTQMRELPVLDSAAFNVECFKKYACNNEYWETF |
| | | | KENPIRLTEENVVNYITKLKGPKAAALFAKTHNLNMLQD |
| | | | IPMDRFVMDLKRDVKVTPGTKHTEERPKVQVIQAADPL |
| | | | ATAYLCGIHRELVRRLNAVLLPNIHTLFDMSAEDFDAIIA |
| | | | EHFQPGDCVLETDIASFDKSEDDAMALTALMILEDLGVD |
| | | | AELLTLIEAAFGEISSIHLPTKTKFKFGAMMKSGMFLTLF |
| | | | VNTVINIVIASRVLRERLTGSPCAAFIGDDNIVKGVKSDK |
| | | | LMADRCATWLNMEVKIIDAVVGEKAPYFCGGFILCDSVT |
| | | | GTACRVADPLKRLFKLGKPLAADDEHDDDRRRALHEES |
| | | | TRWNRVGILSELCKAVESRYETVGTSIIVMAMTTLASSV |
| | | | KSFSYLRGAPITLYG* |
| STARR^{TM} | intergenic | nucleotide | CCTGAATGGACTACGACATAGTCTAGTCCGCCAAGGC |
| (SEQ ID NO: | region | | CGCCACC |
| 52) | | | |
| STARR^{TM} | transgene | nucleotide | n/a (depends on gene of our interest) |
| | ORF | | |
| STARR^{TM} | 3′ UTR | nucleotide | ACTCGAGTATGTTACGTGCAAAGGTGATTGTCACCCCC |
| (SEQ ID NO: | | | CGAAAGACCATATTGTGACACACCCTCAGTATCACGC |
| 53) | | | CCAAACATTTACAGCCGCGGTGTCAAAAACCGCGTGG |
| | | | ACGTGGTTAACATCCCTGCTGGGAGGATCAGCCGTAA |
| | | | TTATTATAATTGGCTTGGTGCTGGCTACTATTGTGGCC |
| | | | ATGTACGTGCTGACCAACCAGAAACATAATTGAATAC |
| | | | AGCAGCAATTGGCAAGCTGCTTACATAGAACTCGCGG |
| | | | CGATTGGCATGCCGCCTTAAAATTTTTATTTTATTTTTT |
| | | | CTTTTCTTTTCCGAATCGGATTTTGTTTTTAATATTTCA |
| | | | AAAAAAAAAAAAAAAAAAAAAAAATCTAGAAAAAAA |
| | | | AAAAAAAAAAAAAAAAAAAAAAAAAAAAAAAAAAA |
| | | | AAAAAAAAAAAAAAAAAAAAAAAAAAAAAAAAAAA |
| | | | AAAAAAAAAAAAAAAAAAAAAA |
| Comparitive | 5' UTR | nucleotide | unknown |
| Original | non- | nucleotide | ATGCCCGAGAAGGTGCACGTGGACATCGAGGAGGACA |
| (SEQ ID NO: | structural | | GCCCCTTCCTGAGGGCCCTGCAGAGGAGCTTCCCACA |
| 54) | gene ORF | | GTTCGAAGTGGAGGCCAAGCAGGTGACCGACAACGAC |
| | | | CACGCCAACGCCAGGGCCTTCAGCCACCTGGCCAGCA |
| | | | AGCTGATCGAGACCGAGGTGGACCCCAGCGACACCAT |
| | | | CCTGGACATCGGCAGCGCCCCAGCCAGGAGAATGTAC |
| | | | AGCAAGCACAAGTACCACTGCATCTGCCCCATGAGGT |
| | | | GCGCCGAGGACCCCGACAGGCTGTACAAGTACGCCAC |
| | | | CAAACTGAAGAAGAACTGCAAGGAGATCACCGACAA |
| | | | GGAGCTGGACAAGAAAATGAAGGAGCTGGCCGCCGTG |
| | | | ATGAGCGACCCCGACCTGGAGACCGAGACAATGTGCC |
| | | | TGCACGACGACGAGAGCTGCAGGTACGAGGGCCAGGT |
| | | | GGCCGTCTACCAGGACGTGTACGCCGTCGACGGCCCC |
| | | | ACCAGCCTGTACCACCAGGCCAACAAGGGCGTGAGGG |
| | | | TGGCCTACTGGATCGGCTTCGACACCACACCCTTCATG |
| | | | TTCAAGAACCTGGCCGGCGCCTACCCCAGCTACAGCA |
| | | | CCAACTGGGCCGACGAGACCGTGCTGACCGCCAGGAA |
| | | | CATCGGCCTGTGCAGCAGCGACGTGATGGAGAGGAGC |
| | | | CGGAGAGGCATGAGCATCCTGAGGAAGAAATACCTGA |
| | | | AGCCCAGCAACAACGTGCTGTTCAGCGTGGGCAGCAC |
| | | | CATCTACCACGAGAAGAGGGACCTGCTCAGGAGCTGG |
| | | | CACCTGCCCAGCGTGTTCCACCTGAGGGGCAAGCAGA |
| | | | ACTACACCTGCAGGTGCGAGACCATCGTGAGCTGCGA |
| | | | CGGCTACGTGGTGAAGAGGATCGCCATCAGCCCCGGC |
| | | | CTGTACGGCAAGCCCAGCGGCTACGCCGCTACAATGC |
| | | | ACAGGGAGGGCTTCCTGTGCTGCAAGGTGACCGACAC |
| | | | CCTGAACGGCGAGAGGGTGAGCTTCCCCGTGTGCACC |
| | | | TACGTGCCCGCCACCCTGTGCGACCAGATGACCGGCA |
| | | | TCCTGGCCACCGACGTGAGCGCCGACGACGCCCAGAA |
| | | | GCTGCTCGTGGGCCTGAACCAGAGGATCGTGGTCAAC |
| | | | GGCAGGACCCAGAGGAACACCAACACAATGAAGAAC |
| | | | TACCTGCTGCCCGTGGTGGCCCAGGCTTTCGCCAGGTG |
| | | | GGCCAAGGAGTACAAGGAGGACCAGGAAGACGAGAG |
| | | | GCCCCTGGGCCTGAGGGACAGGCAGCTGGTGATGGGC |
| | | | TGCTGCTGGGCCTTCAGGCGGCACAAGATCACCAGCA |
| | | | TCTACAAGAGGCCCGACACCCAGACCATCATCAAGGT |
| | | | GAACAGCGACTTCCACAGCTTCGTGCTGCCCAGGATC |
| | | | GGCAGCAACACCCTGGAGATCGGCCTGAGGACCCGGA |
| | | | TCAGGAAGATGCTGGAGGAACACAAGGAGCCCAGCCC |
| | | | ACTGATCACCGCCGAGGACGTGCAGGAGGCCAAGTGC |
| | | | GCTGCCGACGAGGCCAAGGAGGTGAGGGAGGCCGAG |
| | | | GAACTGAGGGCCGCCCTGCCACCCCTGGCTGCCGACG |
| | | | TGGAGGAACCCACCCTGGAAGCCGACGTGGACCTGAT |
| | | | GCTGCAGGAGGCCGGCGCCGGAAGCGTGGAGACACCC |
| | | | AGGGGCCTGATCAAGGTGACCAGCTACGACGGCGAGG |
| | | | ACAAGATCGGCAGCTACGCCGTGCTGAGCCCACAGGC |
| | | | CGTGCTGAAGTCCGAGAAGCTGAGCTGCATCCACCCA |
| | | | CTGGCCGAGCAGGTGATCGTGATCACCCACAGCGGCA |
| | | | GGAAGGGCAGGTACGCCGTGGAGCCCTACCACGGCAA |
| | | | GGTGGTCGTGCCCGAGGGCCACGCCATCCCCGTGCAG |
| | | | GACTTCCAGGCCCTGAGCGAGAGCGCCACCATCGTGT |
| | | | ACAACGAGAGGGAGTTCGTGAACAGGTACCTGCACCA |
| | | | TATCGCCACCCACGGCGGAGCCCTGAACACCGACGAG |
| | | | GAATACTACAAGACCGTGAAGCCCAGCGAGCACGACG |
| | | | GCGAGTACCTGTACGACATCGACAGGAAGCAGTGCGT |
| | | | GAAGAAAGAGCTGGTGACCGGCCTGGGACTGACCGGC |
| | | | GAGCTGGTGGACCCACCCTTCCACGAGTTCGCCTACGA |
| | | | GAGCCTGAGGACCAGACCCGCCGCTCCCTACCAGGTG |
| | | | CCCACCATCGGCGTGTACGGCGTGCCCGGCAGCGGAA |
| | | | AGAGCGGCATCATCAAGAGCGCCGTGACCAAGAAAGA |
| | | | CCTGGTGGTCAGCGCCAAGAAAGAGAACTGCGCCGAG |
| | | | ATCATCAGGGACGTGAAGAAGATGAAAGGCCTGGACG |
| | | | TGAACGCGCGCACCGTGGACAGCGTGCTGCTGAACGG |
| | | | CTGCAAGCACCCCGTGGAGACCCTGTACATCGACGAG |
| | | | GCCTTCGCTTGCCACGCCGGCACCCTGAGGGCCCTGAT |
| | | | CGCCATCATCAGGCCCAAGAAAGCCGTGCTGTGCGGC |
| | | | GACCCCAAGCAGTGCGGCTTCTTCAACATGATGTGCCT |
| | | | GAAGGTGCACTTCAACCACGAGATCTGCACCCAGGTG |
| | | | TTCCACAAGAGCATCAGCAGGCGGTGCACCAAGAGCG |
| | | | TGACCAGCGTCGTGAGCACCCTGTTCTACGACAAGAA |
| | | | AATGAGGACCACCAACCCCAAGGAGACCAAAATCGTG |
| | | | ATCGACACCACAGGCAGCACCAAGCCCAAGCAGGACG |
| | | | ACCTGATCCTGACCTGCTTCAGGGGCTGGGTGAAGCA |
| | | | GCTGCAGATCGACTACAAGGGCAACGAGATCATGACC |
| | | | GCCGCTGCCAGCCAGGGCCTGACCAGGAAGGGCGTGT |
| | | | ACGCCGTGAGGTACAAGGTGAACGAGAACCCACTGTA |
| | | | CGCTCCCACCAGCGAGCACGTGAACGTGCTGCTGACC |
| | | | AGGACCGAGGACAGGATCGTGTGGAAGACCCTGGCCG |
| | | | GCGACCCCTGGATCAAGACCCTGACCGCCAAGTACCC |
| | | | CGGCAACTTCACCGCCACCATCGAAGAGTGGCAGGCC |
| | | | GAGCACGACGCCATCATGAGGCACATCCTGGAGAGGC |
| | | | CCGACCCCACCGACGTGTTCCAGAACAAGGCCAACGT |
| | | | GTGCTGGGCCAAGGCCCTGGTGCCCGTGCTGAAGACC |
| | | | GCCGGCATCGACATGACCACAGAGCAGTGGAACACCG |
| | | | TGGACTACTTCGAGACCGACAAGGCCCACAGCGCCGA |
| | | | GATCGTGCTGAACCAGCTGTGCGTGAGGTTCTTCGGCC |
| | | | TGGACCTGGACAGCGGCCTGTTCAGCGCCCCCACCGT |
| | | | GCCACTGAGCATCAGGAACAACCACTGGGACAACAGC |
| | | | CCCAGCCCAAACATGTACGGCCTGAACAAGGAGGTGG |
| | | | TCAGGCAGCTGAGCAGGCGGTACCCACAGCTGCCCAG |
| | | | GGCCGTGGCCACCGGCAGGGTGTACGACATGAACACC |
| | | | GGCACCCTGAGGAACTACGACCCCAGGATCAACCTGG |
| | | | TGCCCGTGAACAGGCGGCTGCCCCACGCCCTGGTGCT |
| | | | GCACCACAACGAGCACCCACAGAGCGACTTCAGCTCC |
| | | | TTCGTGAGCAAGCTGAAAGGCAGGACCGTGCTGGTCG |
| | | | TGGGCGAGAAGCTGAGCGTGCCCGGCAAGATGGTGGA |
| | | | CTGGCTGAGCGACAGGCCCGAGGCCACCTTCCGGGCC |
| | | | AGGCTGGACCTCGGCATCCCCGGCGACGTGCCCAAGT |
| | | | ACGACATCATCTTCGTGAACGTCAGGACCCCATACAA |
| | | | GTACCACCATTACCAGCAGTGCGAGGACCACGCCATC |
| | | | AAGCTGAGCATGCTGACCAAGAAGGCCTGCCTGCACC |
| | | | TGAACCCCGGAGGCACCTGCGTGAGCATCGGCTACGG |
| | | | CTACGCCGACAGGGCCAGCGAGAGCATCATTGGCGCC |
| | | | ATCGCCAGGCTGTTCAAGTTCAGCAGGGTGTGCAAAC |
| | | | CCAAGAGCAGCCTGGAGGAAACCGAGGTGCTGTTCGT |
| | | | GTTCATCGGCTACGACCGGAAGGCCAGGACCCACAAC |
| | | | CCCTACAAGCTGAGCAGCACCCTGACAAACATCTACA |
| | | | CCGGCAGCAGGCTGCACGAGGCCGGCTGCGCCCCCAG |
| | | | CTACCACGTGGTCAGGGGCGATATCGCCACCGCCACC |
| | | | GAGGGCGTGATCATCAACGCTGCCAACAGCAAGGGCC |
| | | | AGCCCGGAGGCGGAGTGTGCGGCGCCCTGTACAAGAA |
| | | | GTTCCCCGAGAGCTTCGACCTGCAGCCCATCGAGGTG |
| | | | GGCAAGGCCAGGCTGGTGAAGGGCGCCGCTAAGCACA |
| | | | TCATCCACGCCGTGGGCCCCAACTTCAACAAGGTGAG |
| | | | CGAGGTGGAAGGCGACAAGCAGCTGGCCGAAGCCTAC |
| | | | GAGAGCATCGCCAAGATCGTGAACGACAATAACTACA |
| | | | AGAGCGTGGCCATCCCACTGCTCAGCACCGGCATCTTC |
| | | | AGCGGCAACAAGGACAGGCTGACCCAGAGCCTGAACC |
| | | | ACCTGCTCACCGCCCTGGACACCACCGATGCCGACGT |
| | | | GGCCATCTACTGCAGGGACAAGAAGTGGGAGATGACC |
| | | | CTGAAGGAGGCCGTGGCCAGGCGGGAGGCCGTGGAA |
| | | | GAGATCTGCATCAGCGACGACTCCAGCGTGACCGAGC |
| | | | CCGACGCCGAGCTGGTGAGGGTGCACCCCAAGAGCTC |
| | | | CCTGGCCGGCAGGAAGGGCTACAGCACCAGCGACGGC |
| | | | AAGACCTTCAGCTACCTGGAGGGCACCAAGTTCCACC |
| | | | AGGCCGCTAAGGACATCGCCGAGATCAACGCTATGTG |
| | | | GCCCGTGGCCACCGAGGCCAACGAGCAGGTGTGCATG |
| | | | TACATCCTGGGCGAGAGCATGTCCAGCATCAGGAGCA |
| | | | AGTGCCCCGTGGAGGAAAGCGAGGCCAGCACACCACC |
| | | | CAGCACCCTGCCCTGCCTGTGCATCCACGCTATGACAC |
| | | | CCGAGAGGGTGCAGCGGCTGAAGGCCAGCAGGCCCGA |
| | | | GCAGATCACCGTGTGCAGCTCCTTCCCACTGCCCAAGT |
| | | | ACAGGATCACCGGCGTGCAGAAGATCCAGTGCAGCCA |
| | | | GCCCATCCTGTTCAGCCCAAAGGTGCCCGCCTACATCC |
| | | | ACCCCAGGAAGTACCTGGTGGAGACCCCACCCGTGGA |
| | | | CGAGACACCCGAGCCAAGCGCCGAGAACCAGAGCACC |
| | | | GAGGGCACACCCGAGCAGCCACCCCTGATCACCGAGG |
| | | | ACGAGACAAGGACCCGGACCCCAGAGCCCATCATTAT |
| | | | CGAGGAAGAGGAAGAGGACAGCATCAGCCTGCTGAG |
| | | | CGACGGCCCCACCCACCAGGTGCTGCAGGTGGAGGCC |
| | | | GACATCCACGGCCCACCCAGCGTGTCCAGCTCCAGCT |
| | | | GGAGCATCCCACACGCCAGCGACTTCGACGTGGACAG |
| | | | CCTGAGCATCCTGGACACCCTGGAGGGCGCCAGCGTG |
| | | | ACCTCCGGCGCCACCAGCGCCGAGACCAACAGCTACT |
| | | | TCGCCAAGAGCATGGAGTTCCTGGCCAGGCCCGTGCC |
| | | | AGCTCCCAGGACCGTGTTCAGGAACCCACCCCACCCA |
| | | | GCTCCCAGGACCAGGACCCCAAGCCTGGCTCCCAGCA |
| | | | GGGCCTGCAGCAGGACCAGCCTGGTGAGCACCCCACC |
| | | | CGGCGTGAACAGGGTGATCACCAGGGAGGAACTGGAG |
| | | | GCCCTGACACCCAGCAGGACCCCCAGCAGGTCCGTGA |
| | | | GCAGGACTAGTCTGGTGTCCAACCCACCCGGCGTGAA |
| | | | CAGGGTGATCACCAGGGAGGAATTCGAGGCCTTCGTG |
| | | | GCCCAGCAACAGAGACGGTTCGACGCCGGCGCCTACA |
| | | | TCTTCAGCAGCGACACCGGCCAGGGACACCTGCAGCA |
| | | | AAAGAGCGTGAGGCAGACCGTGCTGAGCGAGGTGGTG |
| | | | CTGGAGAGGACCGAGCTGGAAATCAGCTACGCCCCCA |
| | | | GGCTGGACCAGGAGAAGGAGGAACTGCTCAGGAAGA |
| | | | AACTGCAGCTGAACCCCACCCCAGCCAACAGGAGCAG |
| | | | GTACCAGAGCAGGAAGGTGGAGAACATGAAGGCCATC |
| | | | ACCGCCAGGCGGATCCTGCAGGGCCTGGGACACTACC |
| | | | TGAAGGCCGAGGGCAAGGTGGAGTGCTACAGGACCCT |
| | | | GCACCCCGTGCCACTGTACAGCTCCAGCGTGAACAGG |
| | | | GCCTTCTCCAGCCCCAAGGTGGCCGTGGAGGCCTGCA |
| | | | ACGCTATGCTGAAGGAGAACTTCCCCACCGTGGCCAG |
| | | | CTACTGCATCATCCCCGAGTACGACGCCTACCTGGACA |
| | | | TGGTGGACGGCGCCAGCTGCTGCCTGGACACCGCCAG |
| | | | CTTCTGCCCCGCCAAGCTGAGGAGCTTCCCCAAGAAA |
| | | | CACAGCTACCTGGAGCCCACCATCAGGAGCGCCGTGC |
| | | | CCAGCGCCATCCAGAACACCCTGCAGAACGTGCTGGC |
| | | | CGCTGCCACCAAGAGGAACTGCAACGTGACCCAGATG |
| | | | AGGGAGCTGCCCGTGCTGGACAGCGCTGCCTTCAACG |
| | | | TGGAGTGCTTCAAGAAATACGCCTGCAACAACGAGTA |
| | | | CTGGGAGACCTTCAAGGAGAACCCCATCAGGCTGACC |
| | | | GAAGAGAACGTGGTGAACTACATCACCAAGCTGAAGG |
| | | | GCCCCAAGGCCGCTGCCCTGTTCGCTAAGACCCACAA |
| | | | CCTGAACATGCTGCAGGACATCCCAATGGACAGGTTC |
| | | | GTGATGGACCTGAAGAGGGACGTGAAGGTGACACCCG |
| | | | GCACCAAGCACACCGAGGAGAGGCCCAAGGTGCAGGT |
| | | | GATCCAGGCCGCTGACCCACTGGCCACCGCCTACCTGT |
| | | | GCGGCATCCACAGGGAGCTGGTGAGGCGGCTGAACGC |
| | | | CGTGCTGCTGCCCAACATCCACACCCTGTTCGACATGA |
| | | | GCGCCGAGGACTTCGACGCCATCATCGCCGAGCACTT |
| | | | CCAGCCCGGCGACTGCGTGCTGGAGACCGACATCGCC |
| | | | AGCTTCGACAAGAGCGAGGATGACGCTATGGCCCTGA |
| | | | CCGCTCTGATGATCCTGGAGGACCTGGGCGTGGACGC |
| | | | CGAGCTGCTCACCCTGATCGAGGCTGCCTTCGGCGAG |
| | | | ATCAGCTCCATCCACCTGCCCACCAAGACCAAGTTCAA |
| | | | GTTCGGCGCTATGATGAAAAGCGGAATGTTCCTGACC |
| | | | CTGTTCGTGAACACCGTGATCAACATTGTGATCGCCAG |
| | | | CAGGGTGCTGCGGGAGAGGCTGACCGGCAGCCCCTGC |
| | | | GCTGCCTTCATCGGCGACGACAACATCGTGAAGGGCG |
| | | | TGAAAAGCGACAAGCTGATGGCCGACAGGTGCGCCAC |
| | | | CTGGCTGAACATGGAGGTGAAGATCATCGACGCCGTG |
| | | | GTGGGCGAGAAGGCCCCCTACTTCTGCGGCGGATTCA |
| | | | TCCTGTGCGACAGCGTGACCGGCACCGCCTGCAGGGT |
| | | | GGCCGACCCCCTGAAGAGGCTGTTCAAGCTGGGCAAG |
| | | | CCACTGGCCGCTGACGATGAGCACGACGATGACAGGO |
| | | | GGAGGGCCCTGCACGAGGAAAGCACCAGGTGGAACA |
| | | | GGGTGGGCATCCTGAGCGAGCTGTGCAAGGCCGTGGA |
| | | | GAGCAGGTACGAGACCGTGGGCACCAGCATCATCGTG |
| | | | ATGGCTATGACCACACTGGCCAGCTCCGTCAAGAGCTT |
| | | | CTCCTACCTGAGGGGGGCCCCTATAACTCTCTACGGCT |
| | | | AA |
| Comparitive | non- | amino acid | MPEKVHVDIEEDSPFLRALQRSFPQFEVEAKQVTDNDHA |
| (SEQ ID NO: | structural | | NARAFSHLASKLIETEVDPSDTILDIGSAPARRMYSKHKY |
| 55) | gene ORF | | HCICPMRCAEDPDRLYKYATKLKKNCKEITDKELDKKM |
| | | | KELAAVMSDPDLETETMCLHDDESCRYEGQVAVYQDV |
| | | | YAVDGPTSLYHQANKGVRVAYWIGFDTTPFMFKNLAGA |
| | | | YPSYSTNWADETVLTARNIGLCSSDVMERSRRGMSILRK |
| | | | KYLKPSNNVLFSVGSTIYHEKRDLLRSWHLPSVFHLRGK |
| | | | QNYTCRCETIVSCDGYVVKRIAISPGLYGKPSGYAATMH |
| | | | REGFLCCKVTDTLNGERVSFPVCTYVPATLCDQMTGILA |
| | | | TDVSADDAQKLLVGLNQRIVVNGRTQRNTNTMKNYLLP |
| | | | VVAQAFARWAKEYKEDQEDERPLGLRDRQLVMGCCWA |
| | | | FRRHKITSIYKRPDTQTIIKVNSDFHSFVLPRIGSNTLEIGL |
| | | | RTRIRKMLEEHKEPSPLITAEDVQEAKCAADEAKEVREA |
| | | | EELRAALPPLAADVEEPTLEADVDLMLQEAGAGSVETPR |
| | | | GLIKVTSYDGEDKIGSYAVLSPQAVLKSEKLSCIHPLAEQ |
| | | | VIVITHSGRKGRYAVEPYHGKVVVPEGHAIPVQDFQALS |
| | | | ESATIVYNEREFVNRYLHHIATHGGALNTDEEYYKTVKP |
| | | | SEHDGEYLYDIDRKQCVKKELVTGLGLTGELVDPPFHEF |
| | | | AYESLRTRPAAPYQVPTIGVYGVPGSGKSGIIKSAVTKKD |
| | | | LVVSAKKENCAEIIRDVKKMKGLDVNARTVDSVLLNGC |
| | | | KHPVETLYIDEAFACHAGTLRALIAIIRPKKAVLCGDPKQ |
| | | | CGFFNMMCLKVHFNHEICTQVFHKSISRRCTKSVTSVVS |
| | | | TLFYDKKMRTTNPKETKIVIDTTGSTKPKQDDLILTCFRG |
| | | | WVKQLQIDYKGNEIMTAAASQGLTRKGVYAVRYKVNE |
| | | | NPLYAPTSEHVNVLLTRTEDRIVWKTLAGDPWIKTLTAK |
| | | | YPGNFTATIEEWQAEHDAIMRHILERPDPTDVFQNKANV |
| | | | CWAKALVPVLKTAGIDMTTEQWNTVDYFETDKAHSAEI |
| | | | VLNQLCVRFFGLDLDSGLFSAPTVPLSIRNNHWDNSPSPN |
| | | | MYGLNKEVVRQLSRRYPQLPRAVATGRVYDMNTGTLR |
| | | | NYDPRINLVPVNRRLPHALVLHHNEHPQSDFSSFVSKLK |
| | | | GRTVLVVGEKLSVPGKMVDWLSDRPEATFRARLDLGIP |
| | | | GDVPKYDIIFVNVRTPYKYHHYQQCEDHAIKLSMLTKKA |
| | | | CLHLNPGGTCVSIGYGYADRASESIIGAIARLFKFSRVCKP |
| | | | KSSLEETEVLFVFIGYDRKARTHNPYKLSSTLTNIYTGSRL |
| | | | HEAGCAPSYHVVRGDIATATEGVIINAANSKGQPGGGVC |
| | | | GALYKKFPESFDLQPIEVGKARLVKGAAKHIIHAVGPNF |
| | | | NKVSEVEGDKQLAEAYESIAKIVNDNNYKSVAIPLLSTGI |
| | | | FSGNKDRLTQSLNHLLTALDTTDADVAIYCRDKKWEMT |
| | | | LKEAVARREAVEEICISDDSSVTEPDAELVRVHPKSSLAG |
| | | | RKGYSTSDGKTFSYLEGTKFHQAAKDIAEINAMWPVATE |
| | | | ANEQVCMYILGESMSSIRSKCPVEESEASTPPSTLPCLCIH |
| | | | AMTPERVQRLKASRPEQITVCSSFPLPKYRITGVQKIQCS |
| | | | QPILFSPKVPAYIHPRKYLVETPPVDETPEPSAENQSTEGT |
| | | | PEQPPLITEDETRTRTPEPIIIEEEEEDSISLLSDGPTHQVLQ |
| | | | VEADIHGPPSVSSSSWSIPHASDFDVDSLSILDTLEGASVT |
| | | | SGATSAETNSYFAKSMEFLARPVPAPRTVFRNPPHPAPRT |
| | | | RTPSLAPSRACSRTSLVSTPPGVNRVITREELEALTPSRTP |
| | | | SRSVSRTSLVSNPPGVNRVITREEFEAFVAQQQRRFDAGA |
| | | | YIFSSDTGQGHLQQKSVRQTVLSEVVLERTELEISYAPRL |
| | | | DQEKEELLRKKLQLNPTPANRSRYQSRKVENMKAITARR |
| | | | ILQGLGHYLKAEGKVECYRTLHPVPLYSSSVNRAFSSPK |
| | | | VAVEACNAMLKENFPTVASYCIIPEYDAYLDMVDGASC |
| | | | CLDTASFCPAKLRSFPKKHSYLEPTIRSAVPSAIQNTLQNV |
| | | | LAAATKRNCNVTQMRELPVLDSAAFNVECFKKYACNNE |
| | | | YWETFKENPIRLTEENVVNYITKLKGPKAAALFAKTHNL |
| | | | NMLQDIPMDRFVMDLKRDVKVTPGTKHTEERPKVQVIQ |
| | | | AADPLATAYLCGIHRELVRRLNAVLLPNIHTLFDMSAED |
| | | | FDAIIAEHFQPGDCVLETDIASFDKSEDDAMALTALMILE |
| | | | DLGVDAELLTLIEAAFGEISSIHLPTKTKFKFGAMMKSGM |
| | | | FLTLFVNTVINIVIASRVLRERLTGSPCAAFIGDDNIVKGV |
| | | | KSDKLMADRCATWLNMEVKIIDAVVGEKAPYFCGGFIL |
| | | | CDSVTGTACRVADPLKRLFKLGKPLAADDEHDDDRRRA |
| | | | LHEESTRWNRVGILSELCKAVESRYETVGTSIIVMAMTTL |
| | | | ASSVKSFSYLRGAPITLYG* |
| Comparitive | intergenic | nucleotide | unknown |
| | region | | |
| Comparitive | 3′ UTR | nucleotide | unknown |

RNA sequences can include any combination of the RNA sequences listed in Table 4. In some embodiments, RNA sequences of the present disclosure include any combination of the RNA sequences listed in Table 4 in which 0% to 100%, 1% to 100%, 25% to 100%, 50% to 100% and 75% to 100% of the uracil nucleotides of the mRNA sequences are modified. In some embodiments, 1% to 100% of the uracil nucleotides are N1-methylpseudouridine or 5-methoxyuridine. In some embodiments, 100% of the uracil nucleotides are N1-methylpseudouridine. In some embodiments, 100% of the uracil nucleotides are 5-methoxyuridine.

A self-replicating RNA of the disclosure may be obtained by any suitable means. Methods for the manufacture of self-replicating RNA are known in the art and would be readily apparent to a person of ordinary skill. A self-replicating RNA of the disclosure may be prepared according to any available technique including, but not limited to chemical synthesis, in vitro transcription (IVT) or enzymatic or chemical cleavage of a longer precursor, etc.

In some embodiments, a self-replicating RNA of the disclosure is produced from a primary complementary DNA (cDNA) construct. The cDNA constructs can be produced on an RNA template by the action of a reverse transcriptase (e.g., RNA-dependent DNA-polymerase). The process of design and synthesis of the primary cDNA constructs described herein generally includes the steps of gene construction, RNA production (either with or without modifications) and purification. In the IVT method, a target polynucleotide sequence encoding a self-replicating RNA of the disclosure is first selected for incorporation into a vector which will be amplified to produce a cDNA template. Optionally, the target polynucleotide sequence and/or any flanking sequences may be codon optimized. The cDNA template is then used to produce a self-replicating RNA of the disclosure through in vitro transcription (IVT). After production, the self-replicating RNA of the disclosure may undergo purification and clean-up processes. The steps of which are provided in more detail below.

The step of gene construction may include, but is not limited to gene synthesis, vector amplification, plasmid purification, plasmid linearization and clean-up, and cDNA template synthesis and clean-up. Once a protein of interest is selected for production, a primary construct is designed. Within the primary construct, a first region of linked nucleosides encoding the polypeptide of interest may be constructed using an open reading frame (ORF) of a selected nucleic acid (DNA or RNA) transcript. The ORF may comprise the wild type ORF, an isoform, variant or a fragment thereof. As used herein, an “open reading frame” or “ORF” is meant to refer to a nucleic acid sequence (DNA or RNA) which is capable of encoding a polypeptide of interest. ORFs often begin with the start codon, ATG and end with a nonsense or termination codon or signal.

The cDNA templates may be transcribed to produce a self-replicating RNA of the disclosure using an in vitro transcription (IVT) system. The system typically comprises a transcription buffer, nucleotide triphosphates (NTPs), an RNase inhibitor and a polymerase. The NTPs may be selected from, but are not limited to, those described herein including natural and unnatural (modified) NTPs. The polymerase may be selected from, but is not limited to, T7 RNA polymerase, T3 RNA polymerase and mutant polymerases such as, but not limited to, polymerases able to incorporate modified nucleic acids.

The primary cDNA template or transcribed RNA sequence may also undergo capping and/or tailing reactions. A capping reaction may be performed by methods known in the art to add a 5′ cap to the 5′ end of the primary construct. Methods for capping include, but are not limited to, using a Vaccinia Capping enzyme (New England Biolabs, Ipswich, Mass.) or capping at initiation of in vitro transcription, by for example, including a capping agent as part of the IVT reaction. (Nuc. Acids Symp. (2009) 53:129). A poly(A) tailing reaction may be performed by methods known in the art, such as, but not limited to, 2′ O-methyltransferase and by methods as described herein. If the primary construct generated from cDNA does not include a poly-T, it may be beneficial to perform the poly(A)-tailing reaction before the primary construct is cleaned.

The present disclosure also provides expression vectors comprising a nucleotide sequence encoding a self-replicating RNA that is preferably operably linked to at least one regulatory sequence. Regulatory sequences are art-recognized and are selected to direct expression of the encoded polypeptide.

Accordingly, the term regulatory sequence includes promoters, enhancers, and other expression control elements. The design of the expression vector may depend on such factors as the choice of the host cell to be transformed and/or the type of protein desired to be expressed.

The present disclosure also provides polynucleotides (e.g. DNA, RNA, cDNA, mRNA, etc.) directed to a self-replicating RNA of the disclosure that may be operably linked to one or more regulatory nucleotide sequences in an expression construct, such as a vector or plasmid. In certain embodiments, such constructs are DNA constructs. Regulatory nucleotide sequences will generally be appropriate for a host cell used for expression. Numerous types of appropriate expression vectors and suitable regulatory sequences are known in the art for a variety of host cells.

Typically, said one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated by the embodiments of the present disclosure. The promoters may be either naturally occurring promoters, or hybrid promoters that combine elements of more than one promoter.

An expression construct may be present in a cell on an episome, such as a plasmid, or the expression construct may be inserted in a chromosome. In some embodiments, the expression vector contains a selectable marker gene to allow the selection of transformed host cells. Selectable marker genes are well known in the art and will vary with the host cell used.

The present disclosure also provides a host cell transfected with a self-replicating RNA or DNA described herein. The host cell may be any prokaryotic or eukaryotic cell. For example, a polypeptide encoded by a self-replicating RNA may be expressed in bacterial cells such as E. coli, insect cells (e.g., using a baculovirus expression system), yeast, or mammalian cells. Other suitable host cells are known to those skilled in the art.

The present disclosure also provides a host cell comprising a vector comprising a polynucleotide which encodes a self-replicating RNA sequence provided herein.

A host cell transfected with an expression vector comprising a self-replicating RNA of the disclosure can be cultured under appropriate conditions to allow expression of the amplification of the self-replicating RNA and translation of the polypeptide to occur. The polypeptide may be secreted and isolated from a mixture of cells and medium containing the polypeptides. Alternatively, the polypeptides may be retained in the cytoplasm or in a membrane fraction and the cells harvested, lysed and the protein isolated. A cell culture includes host cells, media and other byproducts. Suitable media for cell culture are well known in the art.

The expressed proteins described herein can be isolated from cell culture medium, host cells, or both using techniques known in the art for purifying proteins, including ion-exchange chromatography, gel filtration chromatography, ultrafiltration, electrophoresis, and immunoaffinity purification with antibodies specific for particular epitopes of the polypeptide.

Compositions and Pharmaceutical Compositions

Provided herein, in some embodiments, are compositions comprising any of the nucleic acid molecules provided herein. Compositions provided herein can include a lipid. Any lipid can be included in compositions provided herein. In one aspect, the lipid is an ionizable cationic lipid. Any ionizable cationic lipid can be included in compositions comprising nucleic acid molecules provided herein.

Also provided herein, in some embodiments, are pharmaceutical compositions comprising any of the nucleic acid molecules provided herein and a lipid formulation. Any lipid can be included in lipid formulations of pharmaceutical compositions provided herein. In one aspect, lipid formulations of pharmaceutical compositions provided herein include an ionizable cationic lipid. Exemplary ionizable cationic lipids of compositions and pharmaceutical compositions provided herein include the following:

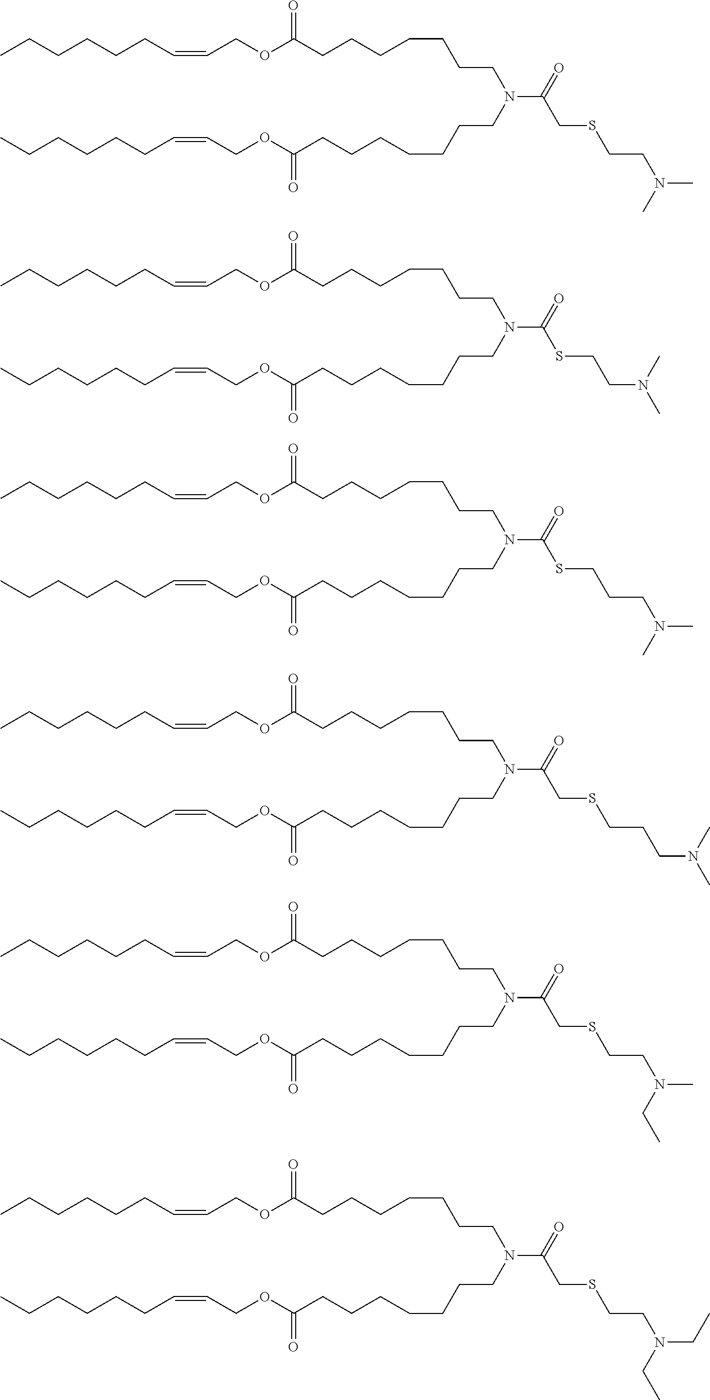

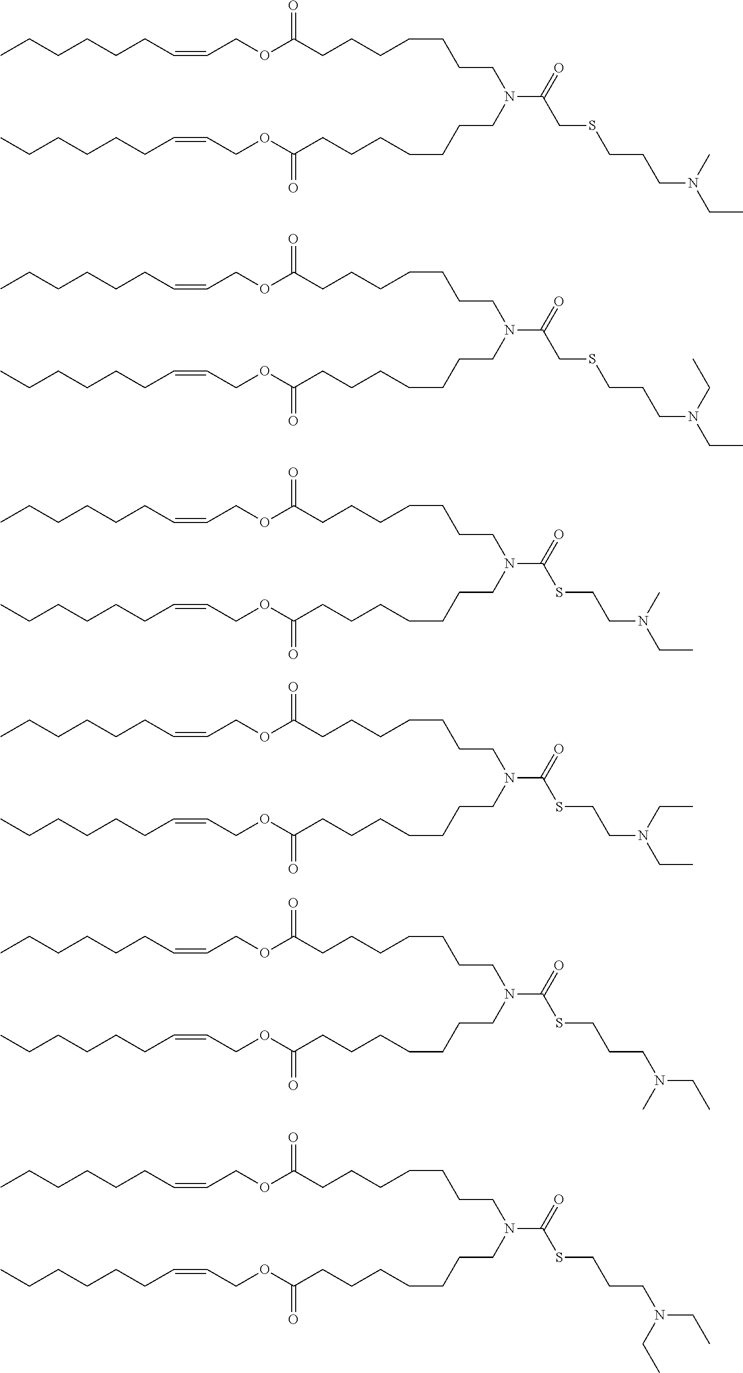

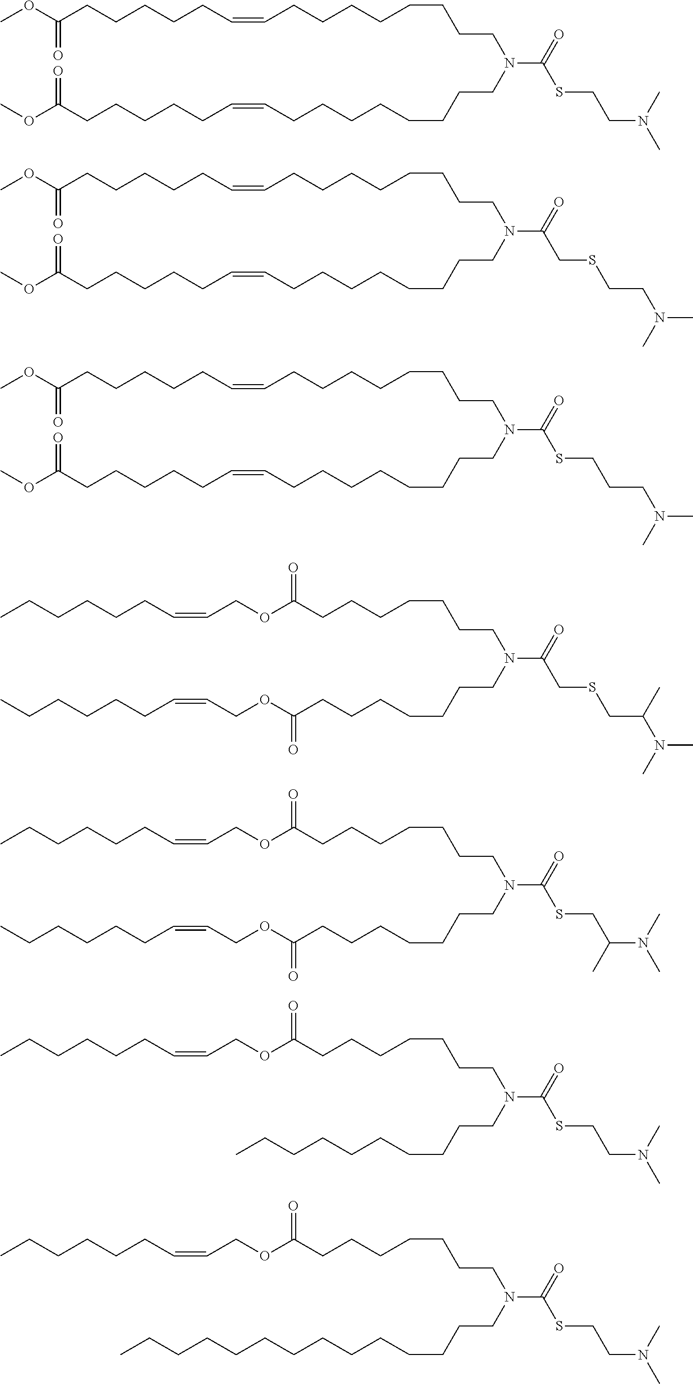

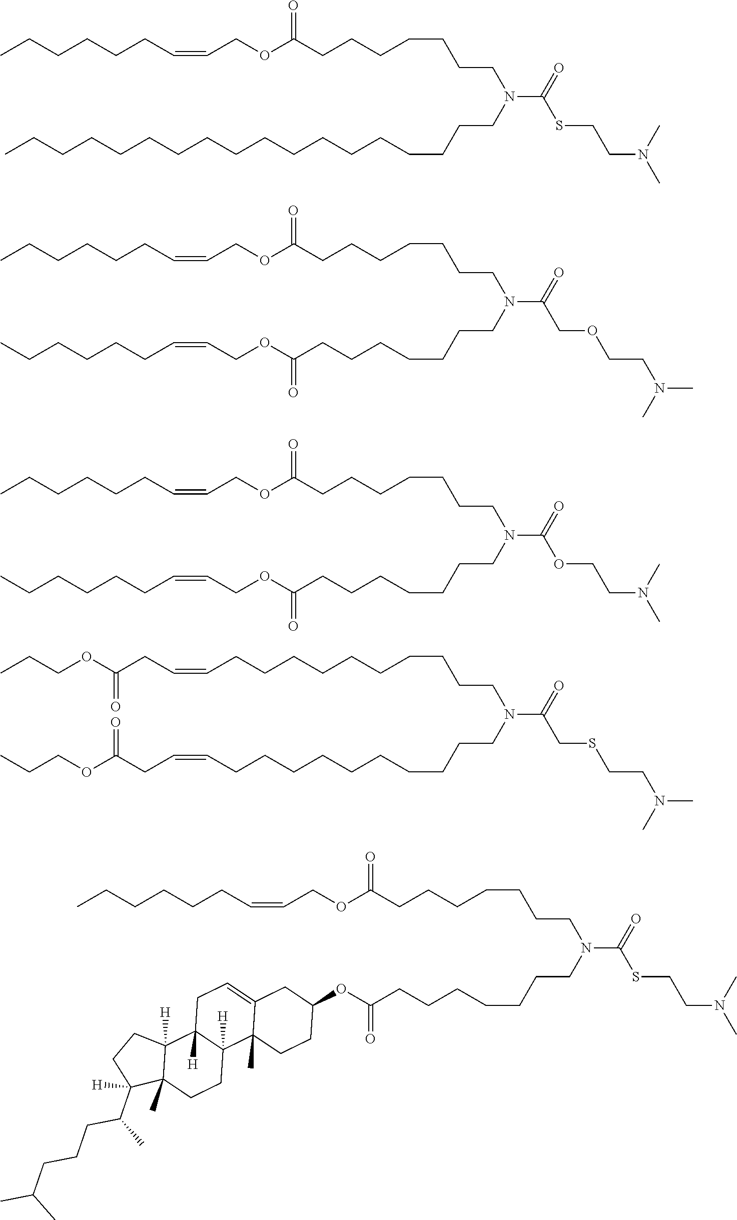

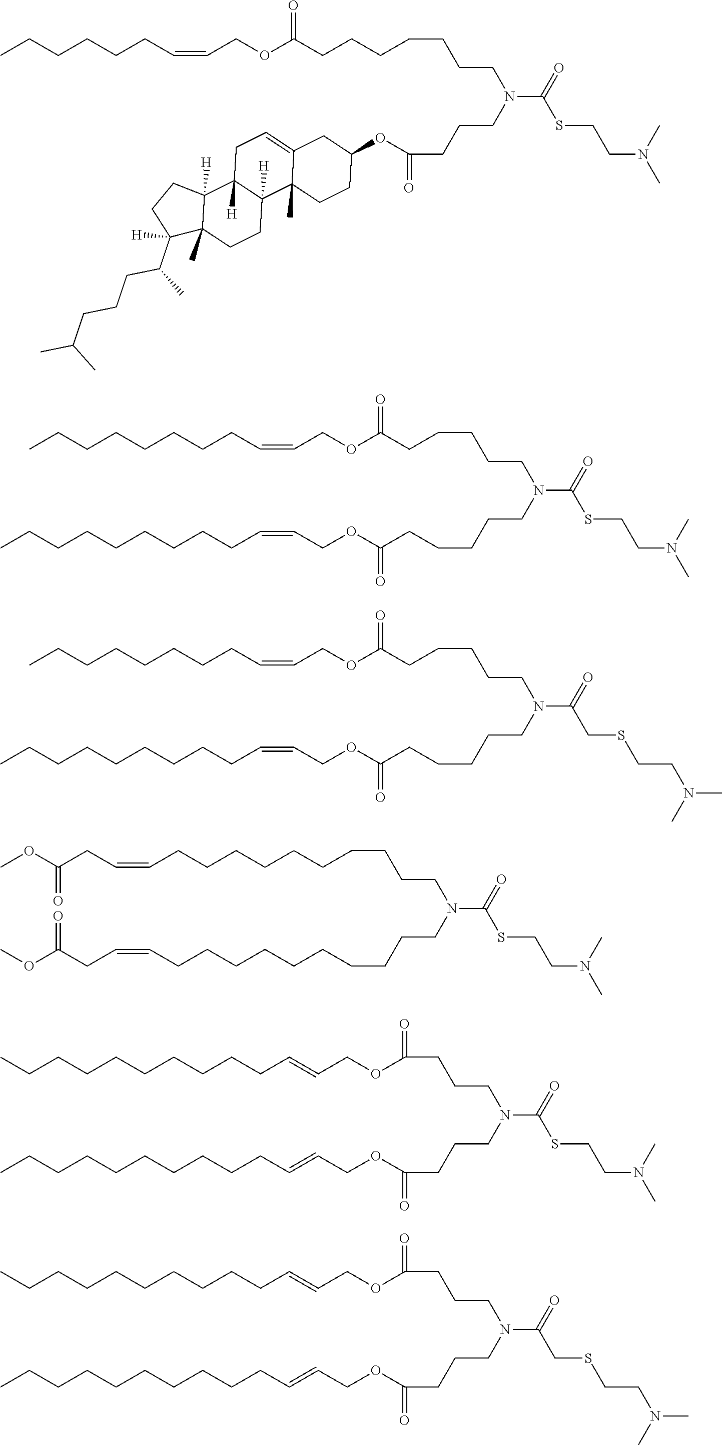

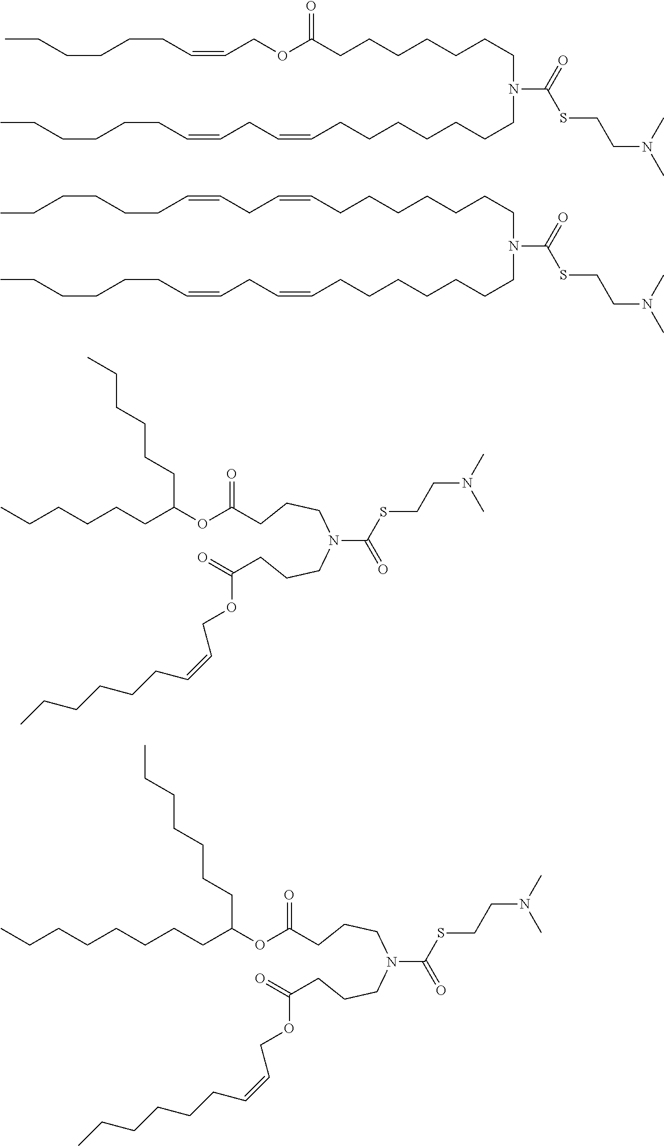

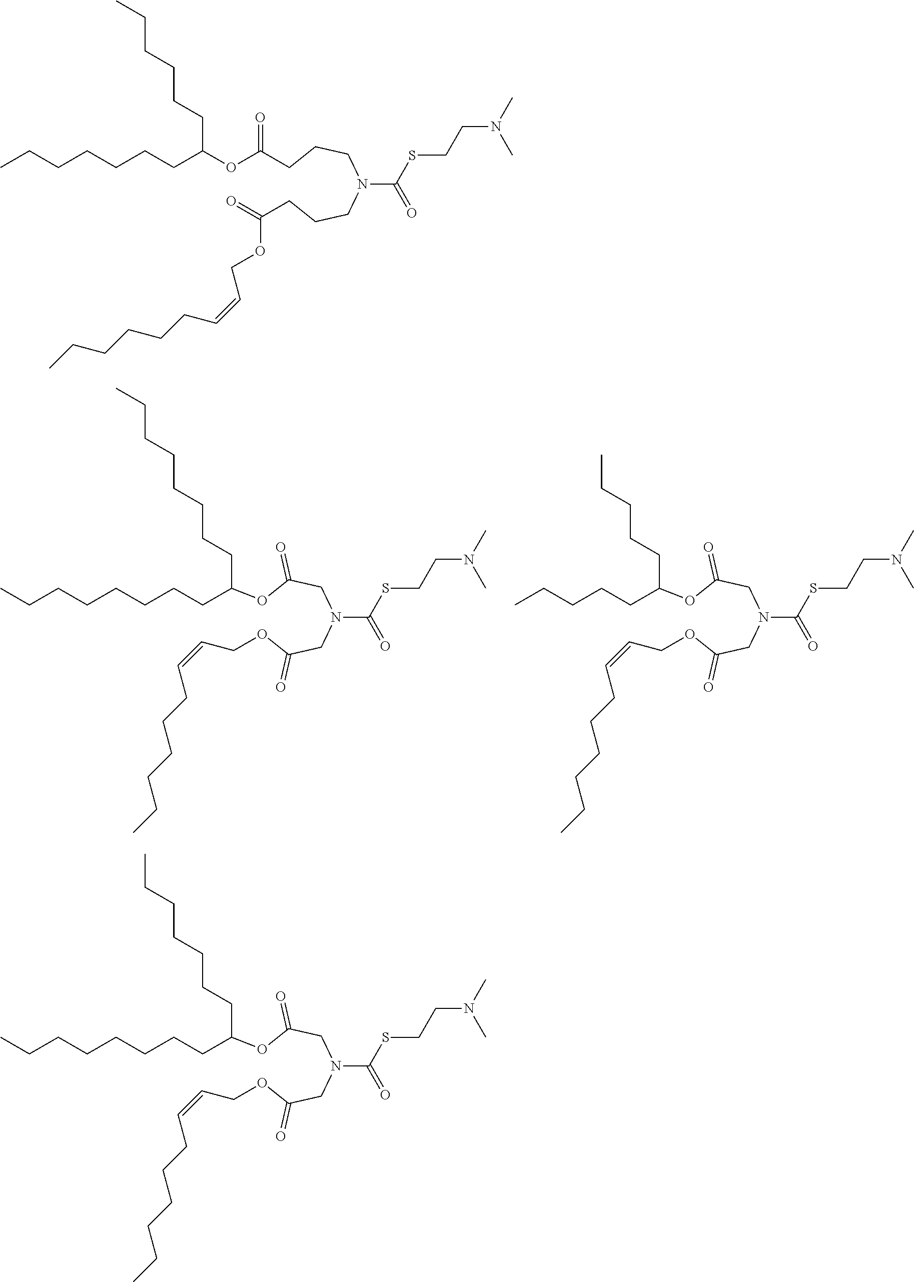

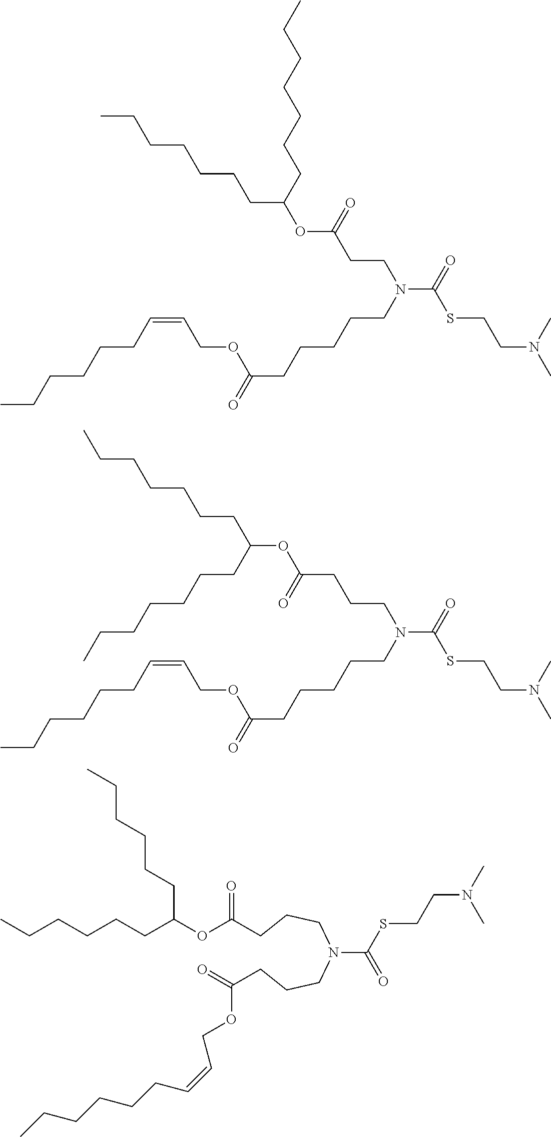

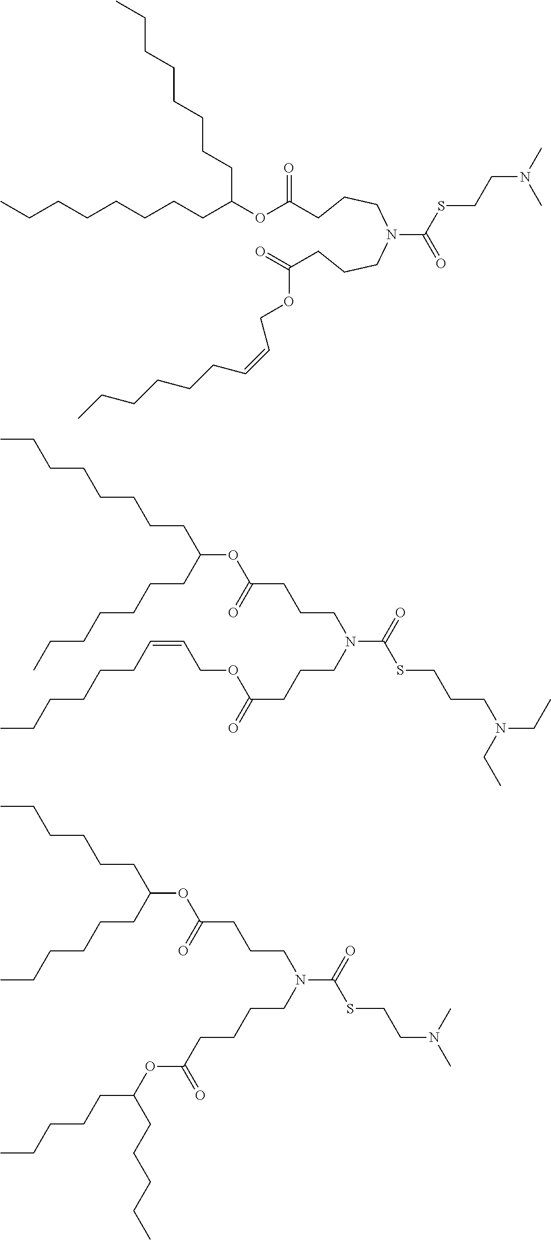

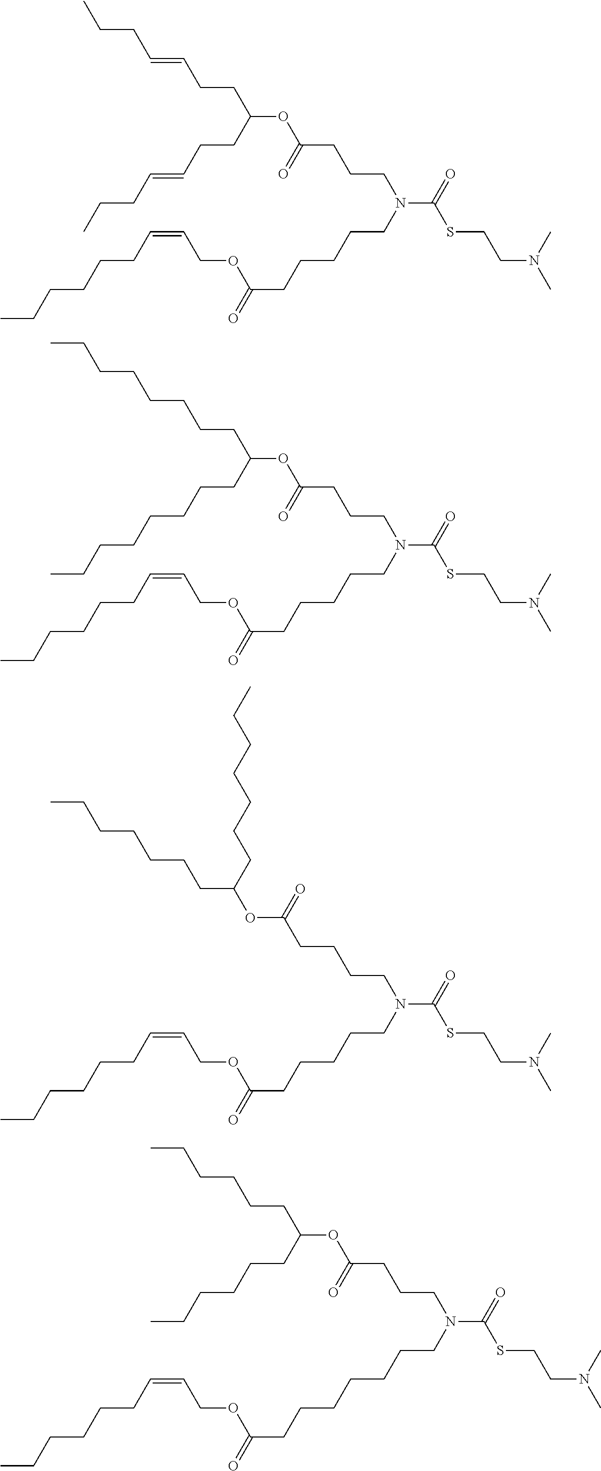

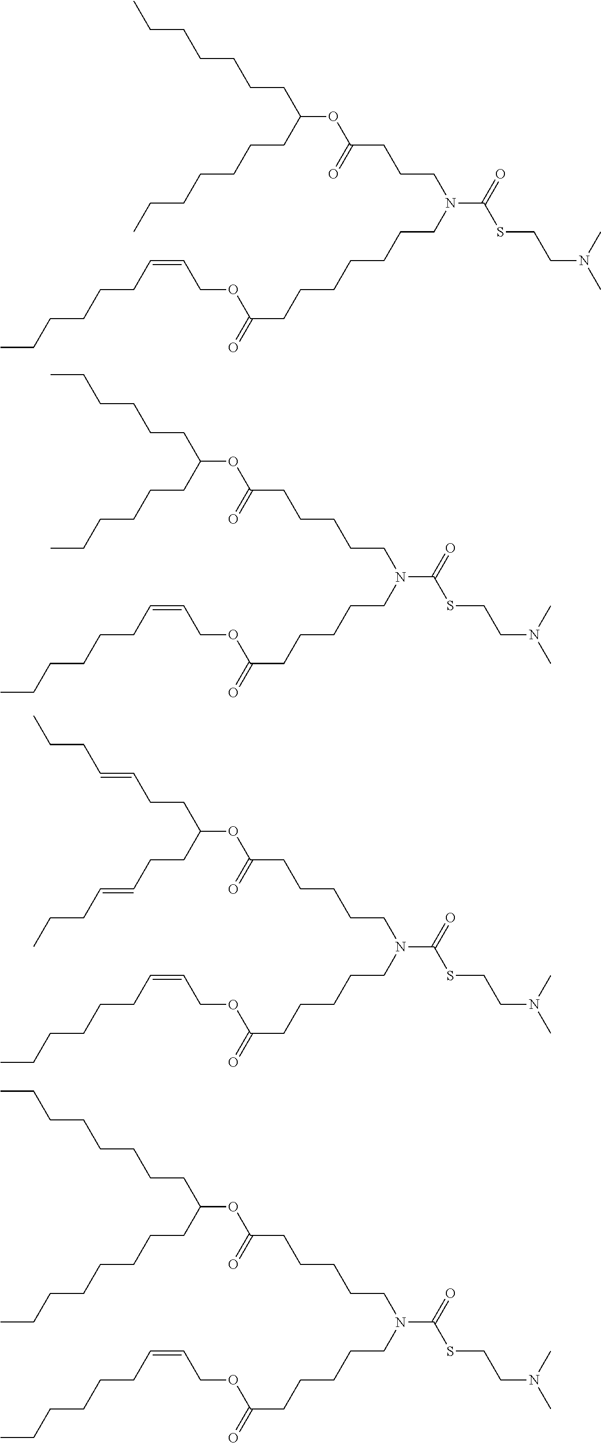

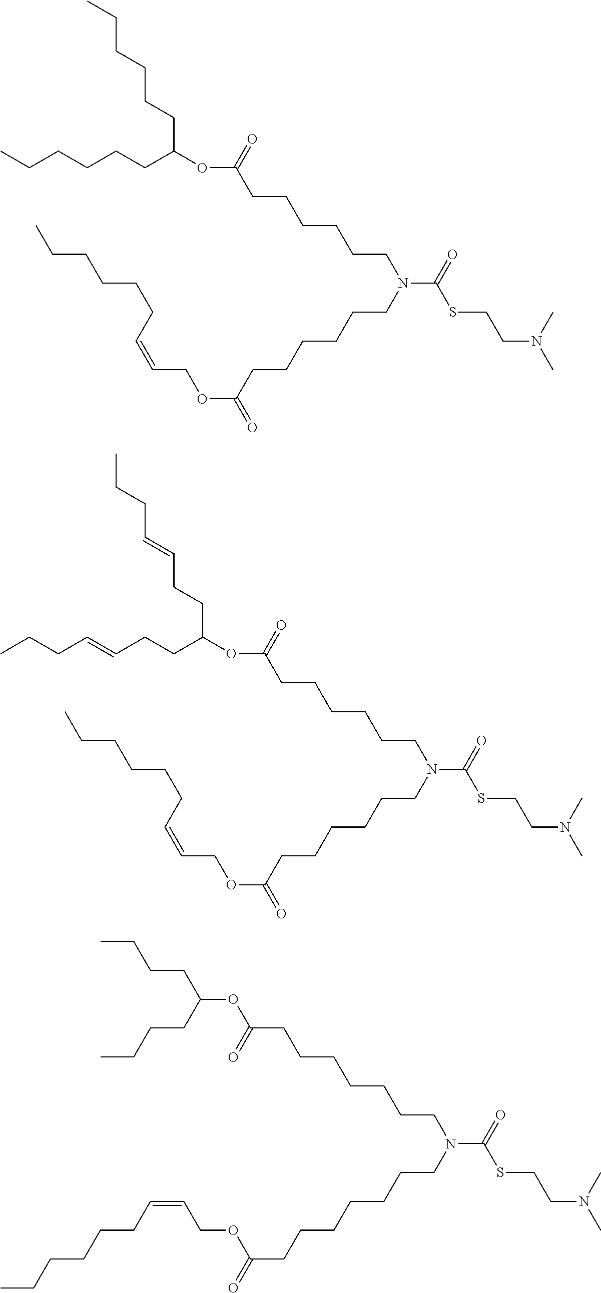

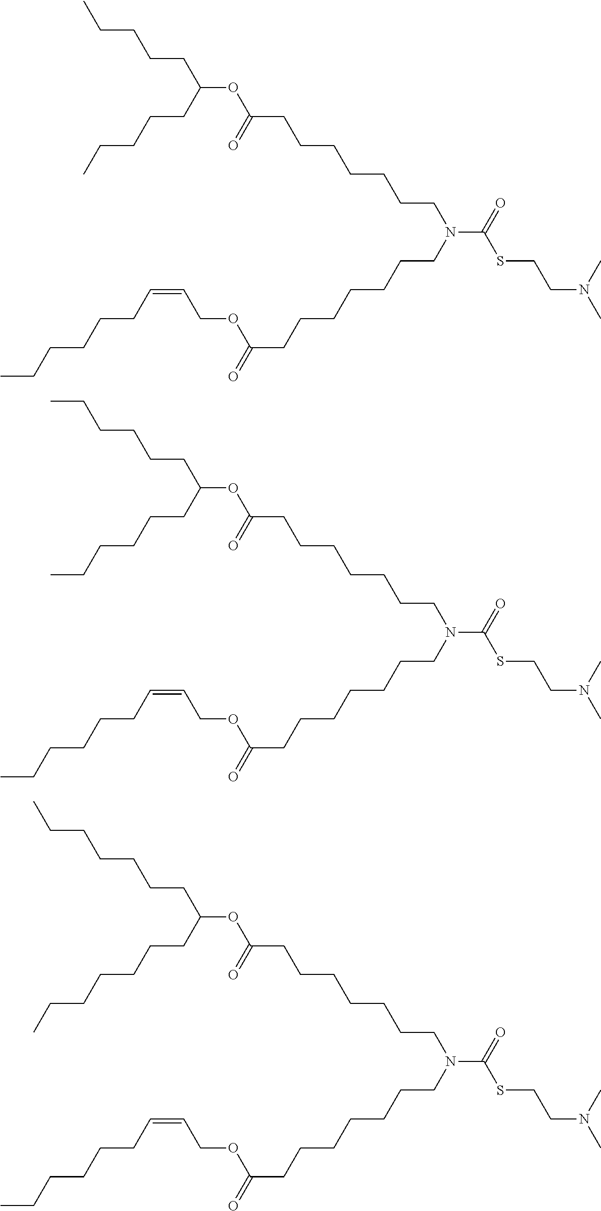

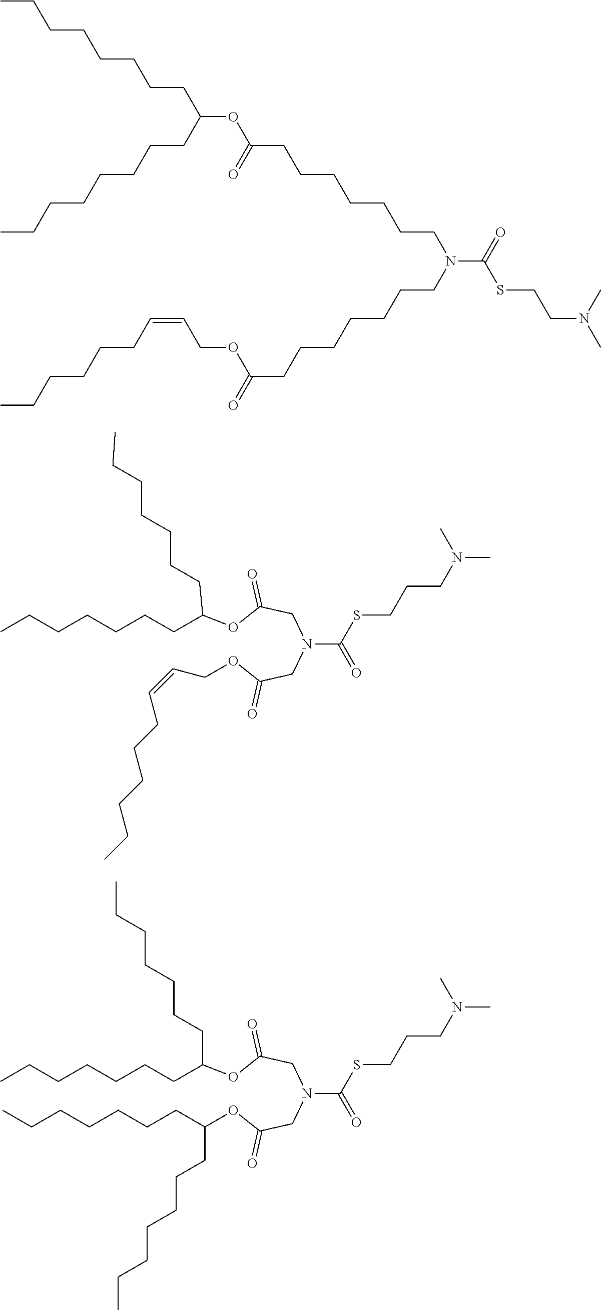

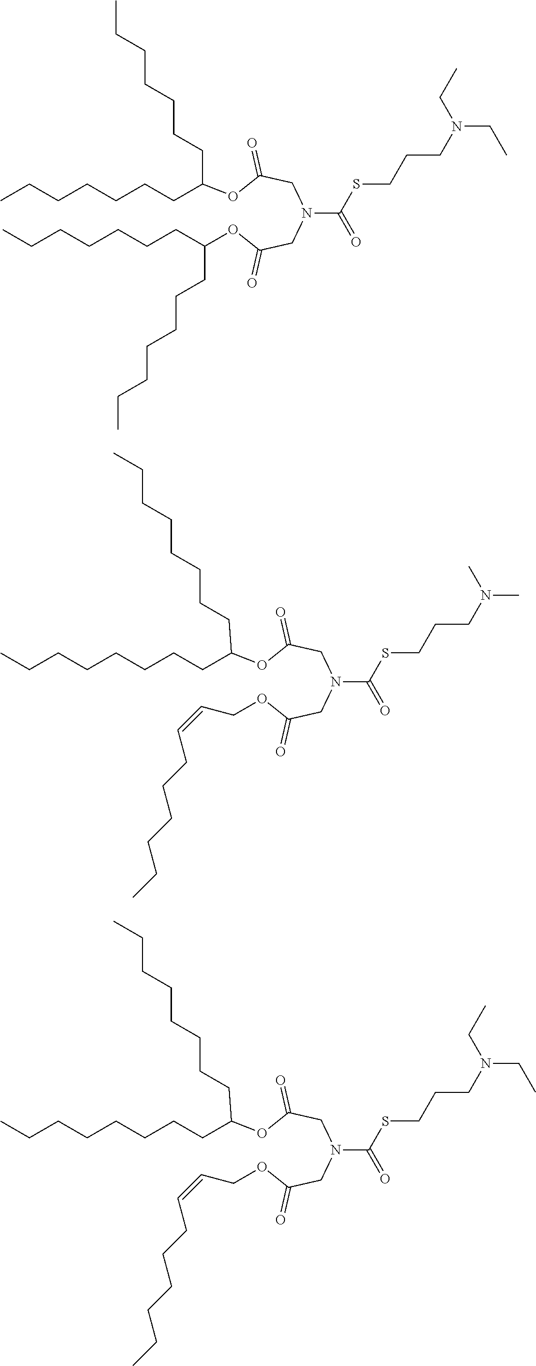

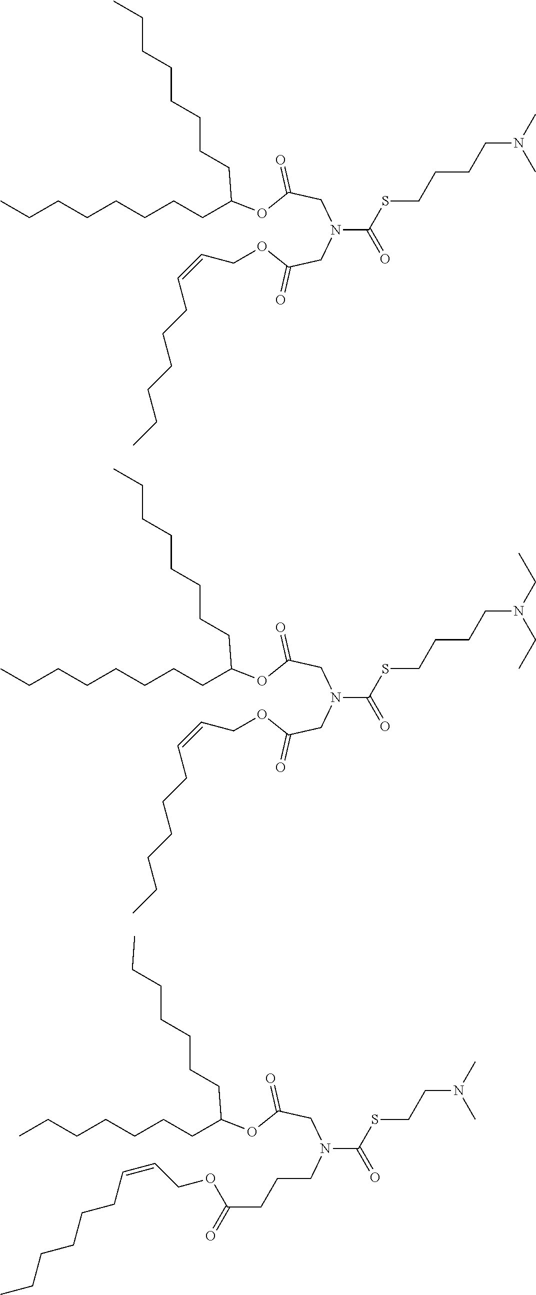

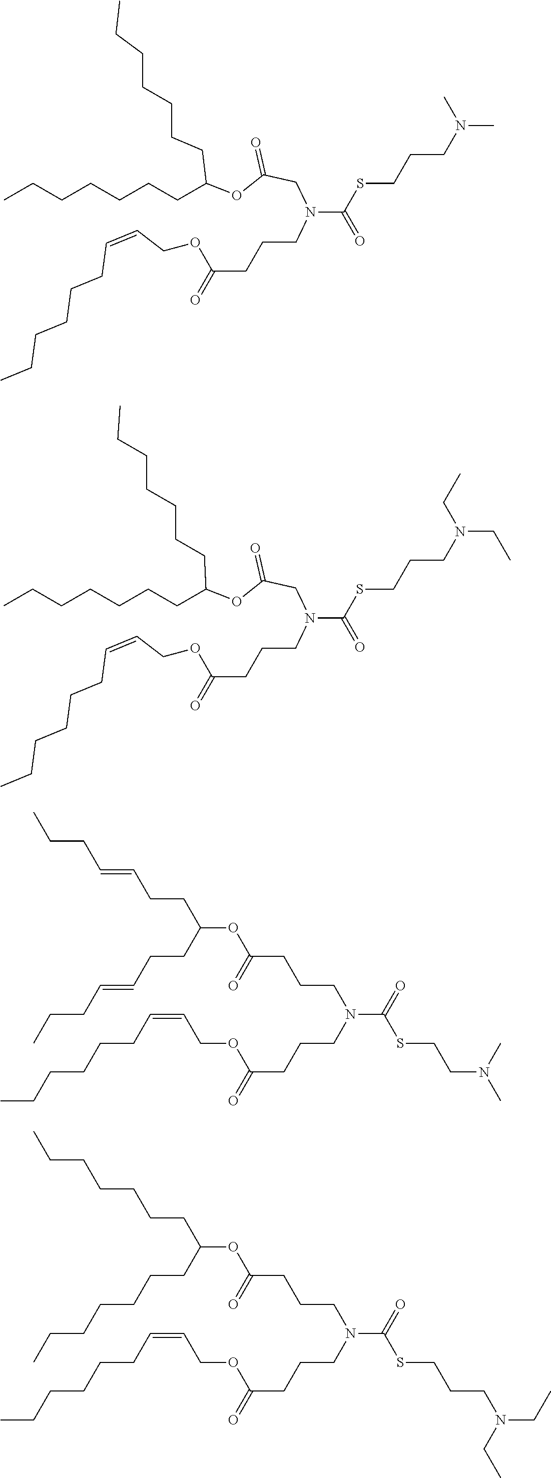

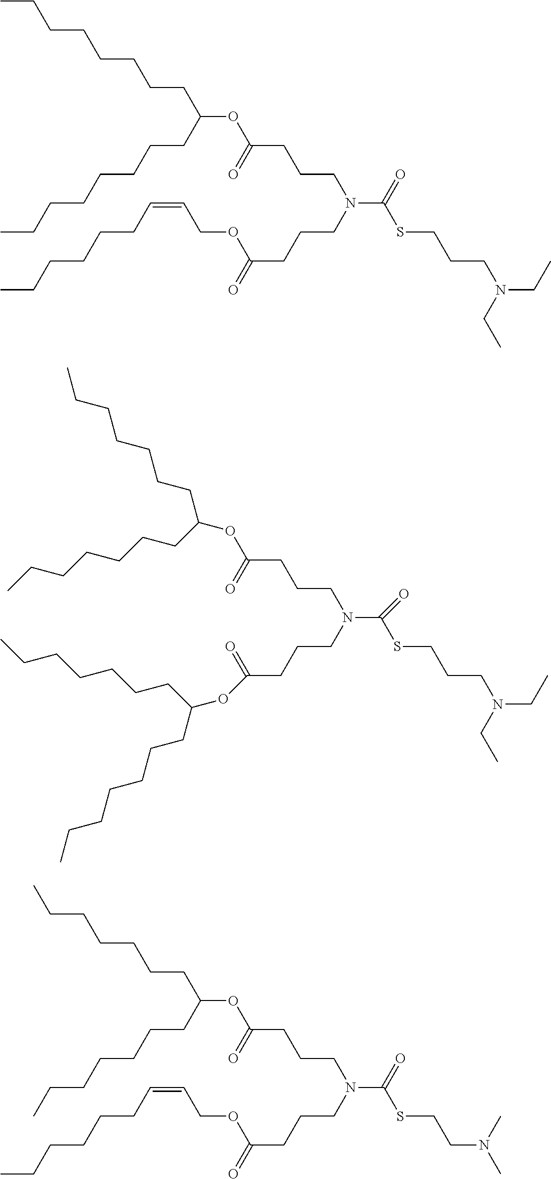

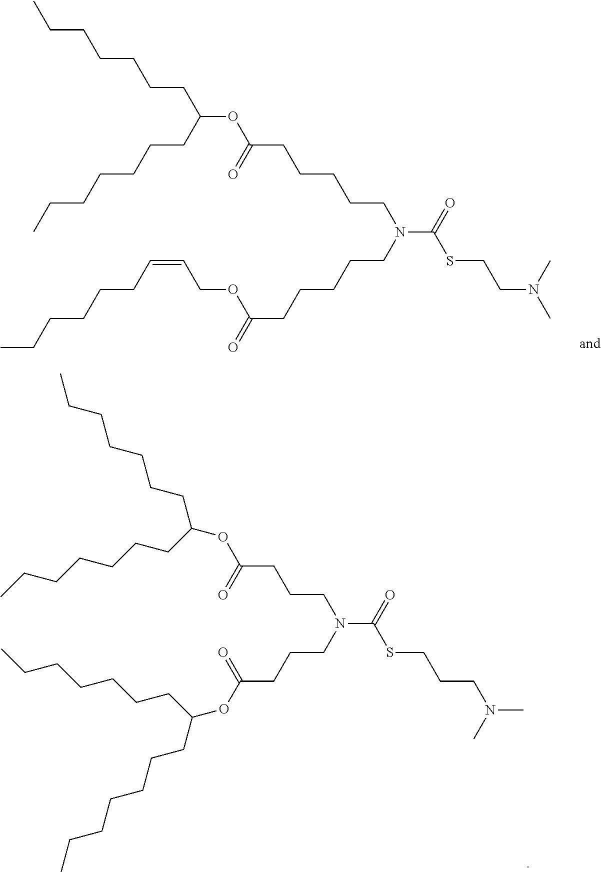

In one aspect, the ionizable cationic lipid of compositions provided herein has a structure of

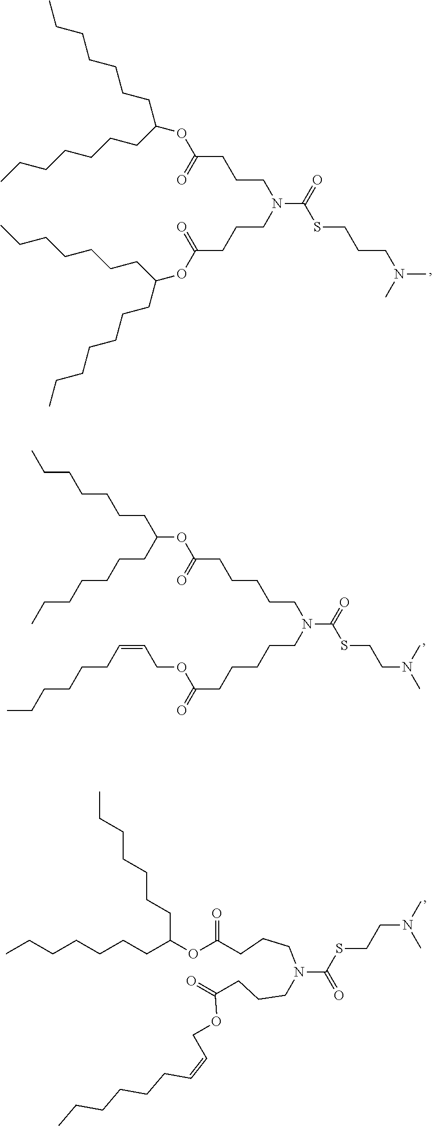

or a pharmaceutically acceptable salt thereof.

In another aspect, the ionizable cationic lipid of compositions provided herein has a structure of

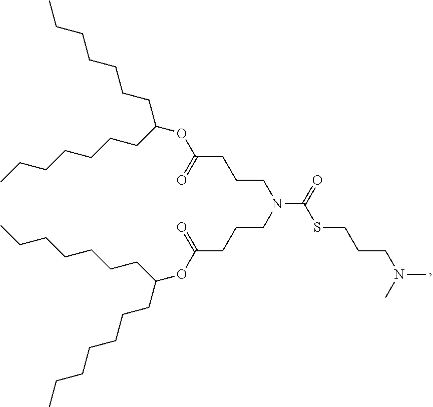 [SMILES: CCCCCCCC(CCCCCCC)OC(=O)CCCN(CCCC(=O)OC(CCCCCCC)CCCCCCC)C(=O)SCCCN(C)C]

or a pharmaceutically acceptable salt thereof.

In one aspect, the ionizable cationic lipid included in lipid formulations of pharmaceutical compositions provided herein has a structure of

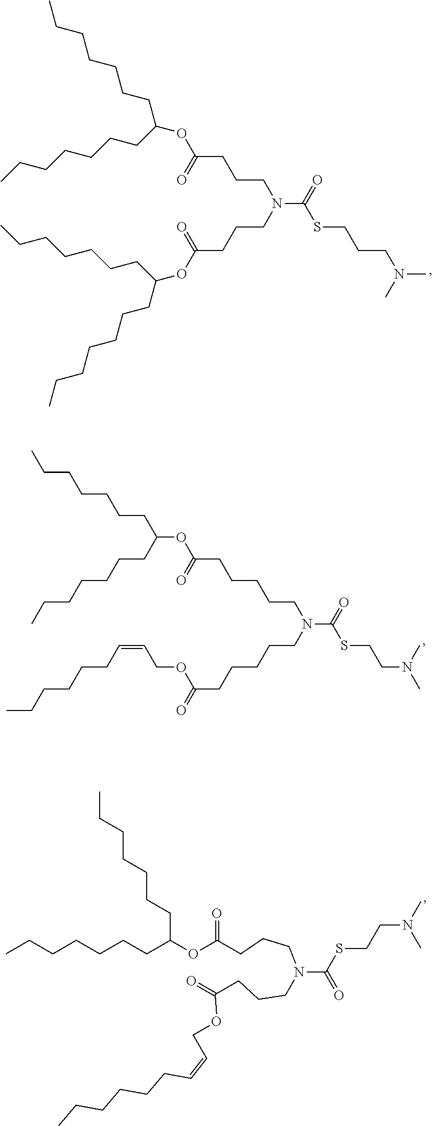

or a pharmaceutically acceptable salt thereof.

In another aspect, the ionizable cationic lipid included in lipid formulations of pharmaceutical compositions provided herein has a structure of

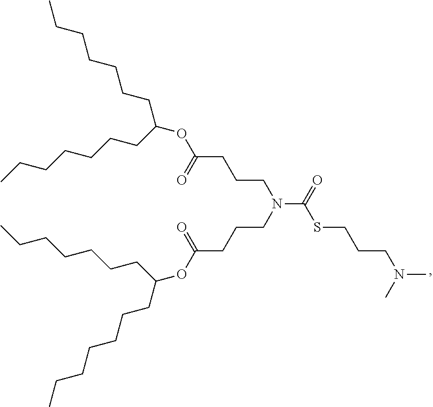 [SMILES: CCCCCCCC(CCCCCCC)OC(=O)CCCN(CCCC(=O)OC(CCCCCCC)CCCCCCC)C(=O)SCCCN(C)C]

or a pharmaceutically acceptable salt thereof.

Lipid Formulations/LNPs

Therapies based on the intracellular delivery of nucleic acids to target cells face both extracellular and intracellular barriers. Indeed, naked nucleic acid materials cannot be easily systemically administered due to their toxicity, low stability in serum, rapid renal clearance, reduced uptake by target cells, phagocyte uptake and their ability in activating the immune response, all features that preclude their clinical development. When exogenous nucleic acid material (e.g., mRNA) enters the human biological system, it is recognized by the reticuloendothelial system (RES) as foreign pathogens and cleared from blood circulation before having the chance to encounter target cells within or outside the vascular system. It has been reported that the half-life of naked nucleic acid in the blood stream is around several minutes (Kawabata K, Takakura Y, Hashida MPharm Res. 1995 June; 12(6):825-30). Chemical modification and a proper delivery method can reduce uptake by the RES and protect nucleic acids from degradation by ubiquitous nucleases, which increase stability and efficacy of nucleic acid-based therapies. In addition, RNAs or DNAs are anionic hydrophilic polymers that are not favorable for uptake by cells, which are also anionic at the surface. The success of nucleic acid-based therapies thus depends largely on the development of vehicles or vectors that can efficiently and effectively deliver genetic material to target cells and obtain sufficient levels of expression in vivo with minimal toxicity.

Moreover, upon internalization into a target cell, nucleic acid delivery vectors are challenged by intracellular barriers, including endosome entrapment, lysosomal degradation, nucleic acid unpacking from vectors, translocation across the nuclear membrane (for DNA), release at the cytoplasm (for RNA), and so on. Successful nucleic acid-based therapy thus depends upon the ability of the vector to deliver the nucleic acids to the target sites inside of the cells in order to obtain sufficient levels of a desired activity such as expression of a gene.

While several gene therapies have been able to successfully utilize a viral delivery vector (e.g., AAV), lipid-based formulations have been increasingly recognized as one of the most promising delivery systems for RNA and other nucleic acid compounds due to their biocompatibility and their ease of large-scale production. One of the most significant advances in lipid-based nucleic acid therapies happened in August 2018 when Patisiran (ALN-TTR02) was the first siRNA therapeutic approved by the Food and Drug Administration (FDA) and by the European Commission (EC). ALN-TTR02 is an siRNA formulation based upon the so-called Stable Nucleic Acid Lipid Particle (SNALP) transfecting technology. Despite the success of Patisiran, the delivery of nucleic acid therapeutics, including mRNA, via lipid formulations is still under ongoing development.

Some art-recognized lipid-formulated delivery vehicles for nucleic acid therapeutics include, according to various embodiments, polymer based carriers, such as polyethyleneimine (PEI), lipid nanoparticles and liposomes, nanoliposomes, ceramide-containing nanoliposomes, multivesicular liposomes, proteoliposomes, both natural and synthetically-derived exosomes, natural, synthetic and semi-synthetic lamellar bodies, nanoparticulates, micelles, and emulsions. These lipid formulations can vary in their structure and composition, and as can be expected in a rapidly evolving field, several different terms have been used in the art to describe a single type of delivery vehicle. At the same time, the terms for lipid formulations have varied as to their intended meaning throughout the scientific literature, and this inconsistent use has caused confusion as to the exact meaning of several terms for lipid formulations. Among the several potential lipid formulations, liposomes, cationic liposomes, and lipid nanoparticles are specifically described in detail and defined herein for the purposes of the present disclosure.

Liposomes

Conventional liposomes are vesicles that consist of at least one bilayer and an internal aqueous compartment. Bilayer membranes of liposomes are typically formed by amphiphilic molecules, such as lipids of synthetic or natural origin that comprise spatially separated hydrophilic and hydrophobic domains (Lasic, Trends Biotechnol., 16: 307-321, 1998). Bilayer membranes of the liposomes can also be formed by amphiphilic polymers and surfactants (e.g., polymerosomes, niosomes, etc.). They generally present as spherical vesicles and can range in size from 20 nm to a few microns. Liposomal formulations can be prepared as a colloidal dispersion or they can be lyophilized to reduce stability risks and to improve the shelf-life for liposome-based drugs. Methods of preparing liposomal compositions are known in the art and would be within the skill of an ordinary artisan.

Liposomes that have only one bilayer are referred to as being unilamellar, and those having more than one bilayer are referred to as multilamellar. The most common types of liposomes are small unilamellar vesicles (SUV), large unilamellar vesicle (LUV), and multilamellar vesicles (MLV). In contrast to liposomes, lysosomes, micelles, and reversed micelles are composed of monolayers of lipids. Generally, a liposome is thought of as having a single interior compartment, however some formulations can be multivesicular liposomes (MVL), which consist of numerous discontinuous internal aqueous compartments separated by several nonconcentric lipid bilayers.

Liposomes have long been perceived as drug delivery vehicles because of their superior biocompatibility, given that liposomes are basically analogs of biological membranes, and can be prepared from both natural and synthetic phospholipids (Int J Nanomedicine. 2014; 9:1833-1843). In their use as drug delivery vehicles, because a liposome has an aqueous solution core surrounded by a hydrophobic membrane, hydrophilic solutes dissolved in the core cannot readily pass through the bilayer, and hydrophobic compounds will associate with the bilayer. Thus, a liposome can be loaded with hydrophobic and/or hydrophilic molecules. When a liposome is used to carry a nucleic acid such as RNA, the nucleic acid will be contained within the liposomal compartment in an aqueous phase.

Cationic Liposomes

Liposomes can be composed of cationic, anionic, and/or neutral lipids. As an important subclass of liposomes, cationic liposomes are liposomes that are made in whole or part from positively charged lipids, or more specifically a lipid that comprises both a cationic group and a lipophilic portion. In addition to the general characteristics profiled above for liposomes, the positively charged moieties of cationic lipids used in cationic liposomes provide several advantages and some unique structural features. For example, the lipophilic portion of the cationic lipid is hydrophobic and thus will direct itself away from the aqueous interior of the liposome and associate with other nonpolar and hydrophobic species. Conversely, the cationic moiety will associate with aqueous media and more importantly with polar molecules and species with which it can complex in the aqueous interior of the cationic liposome. For these reasons, cationic liposomes are increasingly being researched for use in gene therapy due to their favorability towards negatively charged nucleic acids via electrostatic interactions, resulting in complexes that offer biocompatibility, low toxicity, and the possibility of the large-scale production required for in vivo clinical applications. Cationic lipids suitable for use in cationic liposomes are listed herein below.

Lipid Nanoparticles

In contrast to liposomes and cationic liposomes, lipid nanoparticles (LNP) have a structure that includes a single monolayer or bilayer of lipids that encapsulates a compound in a solid phase. Thus, unlike liposomes, lipid nanoparticles do not have an aqueous phase or other liquid phase in its interior, but rather the lipids from the bilayer or monolayer shell are directly complexed to the internal compound thereby encapsulating it in a solid core. Lipid nanoparticles are typically spherical vesicles having a relatively uniform dispersion of shape and size. While sources vary on what size qualifies a lipid particle as being a nanoparticle, there is some overlap in agreement that a lipid nanoparticle can have a diameter in the range of from 10 nm to 1000 nm. However, more commonly they are considered to be smaller than 120 nm or even 100 nm.

For lipid nanoparticle nucleic acid delivery systems, the lipid shell is formulated to include an ionizable cationic lipid which can complex to and associate with the negatively charged backbone of the nucleic acid core. Ionizable cationic lipids with apparent pKa values below about 7 have the benefit of providing a cationic lipid for complexing with the nucleic acid's negatively charged backbone and loading into the lipid nanoparticle at pH values below the pKa of the ionizable lipid where it is positively charged. Then, at physiological pH values, the lipid nanoparticle can adopt a relatively neutral exterior allowing for a significant increase in the circulation half-lives of the particles following i.v. administration. In the context of nucleic acid delivery, lipid nanoparticles offer many advantages over other lipid-based nucleic acid delivery systems including high nucleic acid encapsulation efficiency, potent transfection, improved penetration into tissues to deliver therapeutics, and low levels of cytotoxicity and immunogenicity.

Prior to the development of lipid nanoparticle delivery systems for nucleic acids, cationic lipids were widely studied as synthetic materials for delivery of nucleic acid medicines. In these early efforts, after mixing together at physiological pH, nucleic acids were condensed by cationic lipids to form lipid-nucleic acid complexes known as lipoplexes. However, lipoplexes proved to be unstable and characterized by broad size distributions ranging from the submicron scale to a few microns. Lipoplexes, such as the Lipofectamine® reagent, have found considerable utility for in vitro transfection. However, these first-generation lipoplexes have not proven useful in vivo. The large particle size and positive charge (imparted by the cationic lipid) result in rapid plasma clearance, hemolytic and other toxicities, as well as immune system activation.

In some aspects, nucleic acid molecules provided herein and lipids or lipid formulations provided herein form a lipid nanoparticle (LNP).

In other aspects, nucleic acid molecules provided herein are incorporated into a lipid formulation (i.e., a lipid-based delivery vehicle).

In the context of the present disclosure, a lipid-based delivery vehicle typically serves to transport a desired RNA to a target cell or tissue. The lipid-based delivery vehicle can be any suitable lipid-based delivery vehicle known in the art. In some aspects, the lipid-based delivery vehicle is a liposome, a cationic liposome, or a lipid nanoparticle containing a self-replicating RNA of the disclosure. In some aspects, the lipid-based delivery vehicle comprises a nanoparticle or a bilayer of lipid molecules and a self-replicating RNA of the disclosure. In some aspects, the lipid bilayer further comprises a neutral lipid or a polymer. In some aspects, the lipid formulation comprises a liquid medium. In some aspects, the formulation further encapsulates a nucleic acid. In some aspects, the lipid formulation further comprises a nucleic acid and a neutral lipid or a polymer. In some aspects, the lipid formulation encapsulates the nucleic acid.

The description provides lipid formulations comprising one or more self-replicating RNA molecules encapsulated within the lipid formulation. In some aspects, the lipid formulation comprises liposomes. In some aspects, the lipid formulation comprises cationic liposomes. In some aspects, the lipid formulation comprises lipid nanoparticles.

In some aspects, the self-replicating RNA is fully encapsulated within the lipid portion of the lipid formulation such that the RNA in the lipid formulation is resistant in aqueous solution to nuclease degradation. In other aspects, the lipid formulations described herein are substantially non-toxic to animals such as humans and other mammals.

The lipid formulations of the disclosure also typically have a total lipid:RNA ratio (mass/mass ratio) of from about 1:1 to about 100:1, from about 1:1 to about 50:1, from about 2:1 to about 45:1, from about 3:1 to about 40:1, from about 5:1 to about 45:1, or from about 10:1 to about 40:1, or from about 15:1 to about 40:1, or from about 20:1 to about 40:1; or from about 25:1 to about 45:1; or from about 30:1 to about 45:1; or from about 32:1 to about 42:1; or from about 34:1 to about 42:1. In some aspects, the total lipid:RNA ratio (mass/mass ratio) is from about 30:1 to about 45:1. The ratio may be any value or subvalue within the recited ranges, including endpoints.

The lipid formulations of the present disclosure typically have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm, about 105 nm, about 110 nm, about 115 nm, about 120 nm, about 125 nm, about 130 nm, about 135 nm, about 140 nm, about 145 nm, or about 150 nm, and are substantially non-toxic. The diameter may be any value or subvalue within the recited ranges, including endpoints. In addition, nucleic acids, when present in the lipid nanoparticles of the present disclosure, generally are resistant in aqueous solution to degradation with a nuclease.

In some aspects, the lipid formulations comprise a self-replicating RNA, a cationic lipid (e.g., one or more cationic lipids or salts thereof described herein), a phospholipid, and a conjugated lipid that inhibits aggregation of the particles (e.g., one or more PEG-lipid conjugates). The lipid formulations can also include cholesterol. In one aspect, the cationic lipid is an ionizable cationic lipid.

In the nucleic acid-lipid formulations, the RNA may be fully encapsulated within the lipid portion of the formulation, thereby protecting the nucleic acid from nuclease degradation. In some aspects, a lipid formulation comprising an RNA is fully encapsulated within the lipid portion of the lipid formulation, thereby protecting the nucleic acid from nuclease degradation. In certain aspects, the RNA in the lipid formulation is not substantially degraded after exposure of the particle to a nuclease at 37° C. for at least 20, 30, 45, or 60 minutes. In certain other aspects, the RNA in the lipid formulation is not substantially degraded after incubation of the formulation in serum at 37° C. for at least 30, 45, or 60 minutes or at least 2, 3, 4, 5, 6, 7, 8, 9, 10, 12, 14, 16, 18, 20, 22, 24, 26, 28, 30, 32, 34, or 36 hours. In some aspects, the RNA is complexed with the lipid portion of the formulation. One of the benefits of the formulations of the present disclosure is that the nucleic acid-lipid compositions are substantially non-toxic to animals such as humans and other mammals.

In the context of nucleic acids, full encapsulation may be determined by performing a membrane-impermeable fluorescent dye exclusion assay, which uses a dye that has enhanced fluorescence when associated with nucleic acid. Encapsulation is determined by adding the dye to a lipid formulation, measuring the resulting fluorescence, and comparing it to the fluorescence observed upon addition of a small amount of nonionic detergent. Detergent-mediated disruption of the lipid layer releases the encapsulated nucleic acid, allowing it to interact with the membrane-impermeable dye. Nucleic acid encapsulation may be calculated as E=(I0−I)/I0, where/and I0 refers to the fluorescence intensities before and after the addition of detergent.

In some aspects, the present disclosure provides a nucleic acid-lipid composition comprising a plurality of nucleic acid-liposomes, nucleic acid-cationic liposomes, or nucleic acid-lipid nanoparticles. In some aspects, the nucleic acid-lipid composition comprises a plurality of RNA-liposomes. In some aspects, the nucleic acid-lipid composition comprises a plurality of RNA-cationic liposomes. In some aspects, the nucleic acid-lipid composition comprises a plurality of RNA-lipid nanoparticles.

In some aspects, the lipid formulations comprise RNA that is fully encapsulated within the lipid portion of the formulation, such that from about 30% to about 100%, from about 40% to about 100%, from about 50% to about 100%, from about 60% to about 100%, from about 70% to about 100%, from about 80% to about 100%, from about 90% to about 100%, from about 30% to about 95%, from about 40% to about 95%, from about 50% to about 95%, from about 60% to about 95%, from about 70% to about 95%, from about 80% to about 95%, from about 85% to about 95%, from about 90% to about 95%, from about 30% to about 90%, from about 40% to about 90%, from about 50% to about 90%, from about 60% to about 90%, from about 70% to about 90%, from about 80% to about 90%, or at least about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, or about 99% (or any fraction thereof or range therein) of the particles have the RNA encapsulated therein. The amount may be any value or subvalue within the recited ranges, including endpoints. The RNA included in any RNA-lipid composition or RNA-lipid formulation provided herein can be a self-replicating RNA.

Depending on the intended use of the lipid formulation, the proportions of the components can be varied, and the delivery efficiency of a particular formulation can be measured using assays known in the art.

In some aspects, nucleic acid molecules provided herein are lipid formulated. The lipid formulation is preferably selected from, but not limited to, liposomes, cationic liposomes, and lipid nanoparticles. In one aspect, a lipid formulation is a cationic liposome or a lipid nanoparticle (LNP) comprising:
(a) an RNA of the present disclosure,
(b) a cationic lipid,
(c) an aggregation reducing agent (such as polyethylene glycol (PEG) lipid or PEG-modified lipid),
(d) optionally a non-cationic lipid (such as a neutral lipid), and
(e) optionally, a sterol.

In another aspect, the cationic lipid is an ionizable cationic lipid. Any ionizable cationic lipid can be included in lipid formulations, including exemplary cationic lipids provided herein.

Cationic Lipids

In one aspect, the lipid nanoparticle formulation comprises (i) at least one cationic lipid; (ii) a helper lipid; (iii) a sterol (e.g., cholesterol); and (iv) a PEG-lipid. In another aspect, the cationic lipid is an ionizable cationic lipid. In yet another aspect, the lipid nanoparticle formulation comprises (i) at least one cationic lipid; (ii) a helper lipid; (iii) a sterol (e.g., cholesterol); and (iv) a PEG-lipid, in a molar ratio of about 40-70% ionizable cationic lipid: about 2-15% helper lipid: about 20-45% sterol; about 0.5-5% PEG-lipid. In a further aspect, the cationic lipid is an ionizable cationic lipid.

In one aspect, the lipid nanoparticle formulation consists of (i) at least one cationic lipid; (ii) a helper lipid; (iii) a sterol (e.g., cholesterol); and (iv) a PEG-lipid. In another aspect, the cationic lipid is an ionizable cationic lipid. In yet another aspect, the lipid nanoparticle formulation consists of (i) at least one cationic lipid; (ii) a helper lipid; (iii) a sterol (e.g., cholesterol); and (iv) a PEG-lipid, in a molar ratio of about 40-70% ionizable cationic lipid: about 2-15% helper lipid: about 20-45% sterol; about 0.5-5% PEG-lipid. In a further aspect, the cationic lipid is an ionizable cationic lipid.

In the presently disclosed lipid formulations, the cationic lipid may be, for example, N,N-dioleyl-N,N-dimethylammonium chloride (DODAC), N,N-distearyl-N,N-dimethylammonium bromide (DDAB), 1,2-dioleoyltrimethylammoniumpropane chloride (DOTAP) (also known as N-(2,3-dioleoyloxy)propyl)-N,N,N-trimethylammonium chloride and 1,2-Dioleyloxy-3-trimethylaminopropane chloride salt), N-(1-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-di-y-linolenyloxy-N,N-dimethylaminopropane (γ-DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.Cl), 1,2-Dilinoleoyl-3-trimethylaminopropane chloride salt (DLin-TAP.Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanediol (DOAP), 1,2-Dilinoleyloxo-3-(2-N,N-dimethylamino)ethoxypropane (DLin-EG-DMA), 2,2-Dilinoleyl-4-dimethylaminomethyl-[1,3]-dioxolane (DLin-K-DMA) or analogs thereof, (3aR,5s,6aS)-N,N-dimethyl-2,2-di((9Z,12Z)-octadeca-9,12-dienyl)tetrahydro-3aH-cyclopenta[d][1,3]dioxol-5-amine, (6Z,9Z,28Z,31Z)-heptatriaconta-6,9,28,31-tetraen-19-yl4-(dimethylamino)butanoate (MC3), 1,1′-(2-(4-(2-((2-(bis(2-hydroxydodecyl)amino)ethyl)(2-hydroxydodecyl)amino)ethyl)piperazin-1-yl)ethylazanediyl)didodecan-2-ol (C12-200), 2,2-dilinoleyl-4-(2-dimethylaminoethyl)-[1,3]-dioxolane (DLin-K-C2-DMA), 2,2-dilinoleyl-4-dimethylaminomethyl-[1,3]-dioxolane (DLin-K-DMA), (6Z,9Z,28Z,31Z)-heptatriaconta-6,9,28 31-tetraen-19-yl 4-(dimethylamino) butanoate (DLin-M-C3-DMA), 3-((6Z,9Z,28Z,31Z)-heptatriaconta-6,9,28,31-tetraen-19-yloxy)-N,N-dimethylpropan-1-amine (MC3 Ether), 4-((6Z,9Z,28Z,31 Z)-heptatriaconta-6,9,28,31-tetraen-19-yloxy)-N,N-dimethylbutan-1-amine (MC4 Ether), or any combination thereof. Other cationic lipids include, but are not limited to, N,N-distearyl-N,N-dimethylammonium bromide (DDAB), 3P—(N—(N′,N′-dimethylaminoethane)-carbamoyl)cholesterol (DC-Choi), N-(1-(2,3-dioleyloxy)propyl)-N-2-(sperminecarboxamido)ethyl)-N,N-dimethylammonium trifluoracetate (DOSPA), dioctadecylamidoglycyl carboxyspermine (DOGS), 1,2-dileoyl-sn-3-phosphoethanolamine (DOPE), 1,2-dioleoyl-3-dimethylammonium propane (DODAP), N-(1,2-dimyristyloxyprop-3-yl)-N,N-dimethyl-N-hydroxyethyl ammonium bromide (DMRIE), and 2,2-Dilinoleyl-4-dimethylaminoethyl-11,31-dioxolane (XTC). Additionally, commercial preparations of cationic lipids can be used, such as, e.g., LIPOFECTIN (including DOTMA and DOPE, available from GIBCO/BRL), and Lipofectamine (comprising DOSPA and DOPE, available from GIBCO/BRL).

Other suitable cationic lipids are disclosed in International Publication Nos. WO 09/086558, WO 09/127060, WO 10/048536, WO 10/054406, WO 10/088537, WO 10/129709, and WO 2011/153493; U.S. Patent Publication Nos. 2011/0256175, 2012/0128760, and 2012/0027803; U.S. Pat. No. 8,158,601; and Love et al., PNAS, 107(5), 1864-69, 2010, the contents of which are herein incorporated by reference.

The RNA-lipid formulations of the present disclosure can comprise a helper lipid, which can be referred to as a neutral helper lipid, non-cationic lipid, non-cationic helper lipid, anionic lipid, anionic helper lipid, or a neutral lipid. It has been found that lipid formulations, particularly cationic liposomes and lipid nanoparticles have increased cellular uptake if helper lipids are present in the formulation. (Curr. Drug Metab. 2014; 15(9):882-92). For example, some studies have indicated that neutral and zwitterionic lipids such as 1,2-dioleoylsn-glycero-3-phosphatidylcholine (DOPC), Di-Oleoyl-Phosphatidyl-Ethanoalamine (DOPE) and 1,2-DiStearoyl-sn-glycero-3-PhosphoCholine (DSPC), being more fusogenic (i.e., facilitating fusion) than cationic lipids, can affect the polymorphic features of lipid-nucleic acid complexes, promoting the transition from a lamellar to a hexagonal phase, and thus inducing fusion and a disruption of the cellular membrane. (Nanomedicine (Lond). 2014 January; 9(1):105-20). In addition, the use of helper lipids can help to reduce any potential detrimental effects from using many prevalent cationic lipids such as toxicity and immunogenicity.

Non-limiting examples of non-cationic lipids suitable for lipid formulations of the present disclosure include phospholipids such as lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidic acid, cerebrosides, dicetylphosphate, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dioleoylphosphatidylethanolamine 4-(N-maleimidomethyl)-cyclohexane-1-carboxylate (DOPE-mal), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), lysophosphatidylcholine, dilinoleoylphosphatidylcholine, and mixtures thereof. Other diacylphosphatidylcholine and diacylphosphatidylethanolamine phospholipids can also be used. The acyl groups in these lipids are preferably acyl groups derived from fatty acids having C10-C24 carbon chains, e.g., lauroyl, myristoyl, palmitoyl, stearoyl, or oleoyl.

Additional examples of non-cationic lipids include sterols such as cholesterol and derivatives thereof. As a helper lipid, cholesterol increases the spacing of the charges of the lipid layer interfacing with the nucleic acid making the charge distribution match that of the nucleic acid more closely. (J. R. Soc. Interface. 2012 Mar. 7; 9(68): 548-561). Non-limiting examples of cholesterol derivatives include polar analogues such as 5α-cholestanol, 5α-coprostanol, cholesteryl-(2′-hydroxy)-ethyl ether, cholesteryl-(4′-hydroxy)-butyl ether, and 6-ketocholestanol; non-polar analogues such as 5α-cholestane, cholestenone, 5α-cholestanone, 5α-cholestanone, and cholesteryl decanoate; and mixtures thereof. In some aspects, the cholesterol derivative is a polar analogue such as cholesteryl-(4′-hydroxy)-butyl ether.

In some aspects, the helper lipid present in the lipid formulation comprises or consists of a mixture of one or more phospholipids and cholesterol or a derivative thereof. In other aspects, the neutral lipid present in the lipid formulation comprises or consists of one or more phospholipids, e.g., a cholesterol-free lipid formulation. In yet other aspects, the neutral lipid present in the lipid formulation comprises or consists of cholesterol or a derivative thereof, e.g., a phospholipid-free lipid formulation.

Other examples of helper lipids include nonphosphorous containing lipids such as, e.g., stearylamine, dodecylamine, hexadecylamine, acetyl palmitate, glycerol ricinoleate, hexadecyl stearate, isopropyl myristate, amphoteric acrylic polymers, triethanolamine-lauryl sulfate, alkyl-aryl sulfate polyethyloxylated fatty acid amides, dioctadecyldimethyl ammonium bromide, ceramide, and sphingomyelin.

Other suitable cationic lipids include those having alternative fatty acid groups and other dialkylamino groups, including those, in which the alkyl substituents are different (e.g., N-ethyl-N-methylamino-, and N-propyl-N-ethylamino-). These lipids are part of a subcategory of cationic lipids referred to as amino lipids. In some embodiments of the lipid formulations described herein, the cationic lipid is an amino lipid. In general, amino lipids having less saturated acyl chains are more easily sized, particularly when the complexes must be sized below about 0.3 microns, for purposes of filter sterilization. Amino lipids containing unsaturated fatty acids with carbon chain lengths in the range of C14 to C22 may be used. Other scaffolds can also be used to separate the amino group and the fatty acid or fatty alkyl portion of the amino lipid.

In some embodiments, the lipid formulation comprises the cationic lipid with Formula I according to the patent application PCT/EP2017/064066. In this context, the disclosure of PCT/EP2017/064066 is also incorporated herein by reference.

In some embodiments, amino or cationic lipids of the present disclosure are ionizable and have at least one protonatable or deprotonatable group, such that the lipid is positively charged at a pH at or below physiological pH (e.g., pH 7.4), and neutral at a second pH, preferably at or above physiological pH. Of course, it will be understood that the addition or removal of protons as a function of pH is an equilibrium process, and that the reference to a charged or a neutral lipid refers to the nature of the predominant species and does not require that all of the lipid be present in the charged or neutral form. Lipids that have more than one protonatable or deprotonatable group, or which are zwitterionic, are not excluded from use in the disclosure. In certain embodiments, the protonatable lipids have a pKa of the protonatable group in the range of about 4 to about 11. In some embodiments, the ionizable cationic lipid has a pKa of about 5 to about 7. In some embodiments, the pKa of an ionizable cationic lipid is about 6 to about 7.

In some embodiments, the lipid formulation comprises an ionizable cationic lipid of Formula I:

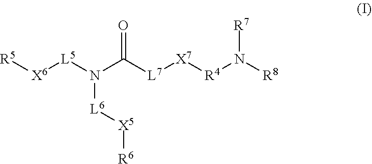

or a pharmaceutically acceptable salt or solvate thereof, wherein R5 and R6 are each independently selected from the group consisting of a linear or branched C1-C31 alkyl, C2-C31 alkenyl or C2-C31 alkynyl and cholesteryl; L5 and L6 are each independently selected from the group consisting of a linear C1-C20 alkyl and C2-C20 alkenyl; X5 is —C(O)O—, whereby —C(O)O—R6 is formed or —OC(O)— whereby —OC(O)—R6 is formed; X6 is —C(O)O— whereby —C(O)O—R5 is formed or —OC(O)— whereby —OC(O)—R5 is formed; X7 is S or O; L7 is absent or lower alkyl; R4 is a linear or branched C1-C6 alkyl; and R7 and R8 are each independently selected from the group consisting of a hydrogen and a linear or branched C1-C6 alkyl.

In some embodiments, X7 is S.

In some embodiments, X5 is —C(O)O—, whereby —C(O)O—R6 is formed and X6 is —C(O)O— whereby —C(O)O—R5 is formed.

In some embodiments, R7 and R8 are each independently selected from the group consisting of methyl, ethyl and isopropyl.

In some embodiments, L5 and L6 are each independently a C1-C10 alkyl. In some embodiments, L5 is C1-C3 alkyl, and L6 is C1-C5 alkyl. In some embodiments, L6 is C1-C2 alkyl. In some embodiments, L5 and L6 are each a linear C7 alkyl. In some embodiments, L5 and L6 are each a linear C9 alkyl.

In some embodiments, R5 and R6 are each independently an alkenyl. In some embodiments, R6 is alkenyl. In some embodiments, R6 is C2-C9 alkenyl. In some embodiments, the alkenyl comprises a single double bond. In some embodiments, R5 and R6 are each alkyl. In some embodiments, R5 is a branched alkyl. In some embodiments, R5 and R6 are each independently selected from the group consisting of a C9 alkyl, C9 alkenyl and C9 alkynyl. In some embodiments, R5 and R6 are each independently selected from the group consisting of a C11 alkyl, C11 alkenyl and C11 alkynyl. In some embodiments, R5 and R6 are each independently selected from the group consisting of a C7 alkyl, C7 alkenyl and C7 alkynyl. In some embodiments, R5 is —CH((CH2)pCH3)2 or —CH((CH2)pCH3)((CH2)p-1CH3), wherein p is 4-8. In some embodiments, p is 5 and L5 is a C1-C3 alkyl. In some embodiments, p is 6 and L5 is a C3 alkyl. In some embodiments, p is 7. In some embodiments, p is 8 and L5 is a C1-C3 alkyl. In some embodiments, R5 consists of —CH((CH2)pCH3)((CH2)p-1CH3), wherein p is 7 or 8.

In some embodiments, R4 is ethylene or propylene. In some embodiments, R4 is n-propylene or isobutylene.

In some embodiments, L7 is absent, R4 is ethylene, X7 is S and R7 and R8 are each methyl. In some embodiments, L7 is absent, R4 is n-propylene, X7 is S and R7 and R8 are each methyl. In some embodiments, L7 is absent, R4 is ethylene, X7 is S and R7 and R8 are each ethyl.

In some embodiments, X7 is S, X5 is —C(O)O—, whereby —C(O)O—R6 is formed, X6 is —C(O)O— whereby —C(O)O—R5 is formed, L5 and L6 are each independently a linear C3-C7 alkyl, L7 is absent, R5 is —CH((CH2)pCH3)2, and R6 is C7-C12 alkenyl. In some further embodiments, p is 6 and R6 is C9 alkenyl.

In some embodiments, the lipid formulation can comprise an ionizable cationic lipid selected from the group consisting of LIPID #1 to LIPID #8:

TABLE 5

| LIPID # | STRUCTURE |
|---|---|
| 1 | 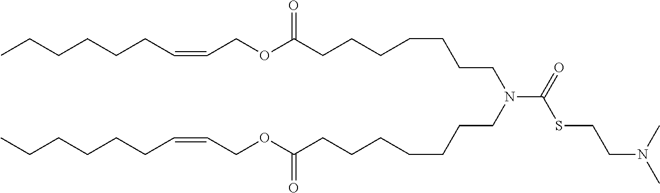 [SMILES: CCCCCC/C=C\COC(=O)CCCCCCCN(CCCCCCCC(=O)OC/C=C\CCCCCC)C(=O)SCCN(C)C] |
| 2 | 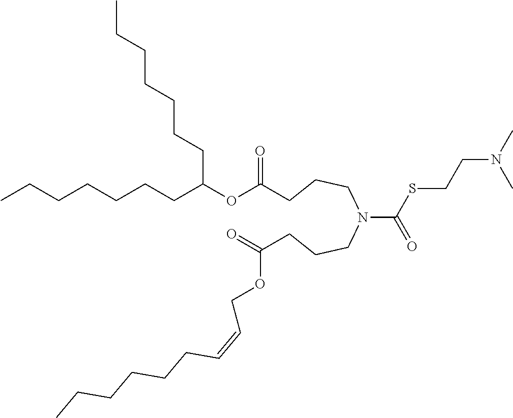 [SMILES: CCCCCC/C=C\COC(=O)CCCN(CCCC(=O)OC(CCCCCCC)CCCCCCC)C(=O)SCCN(C)C] |
| 3 | 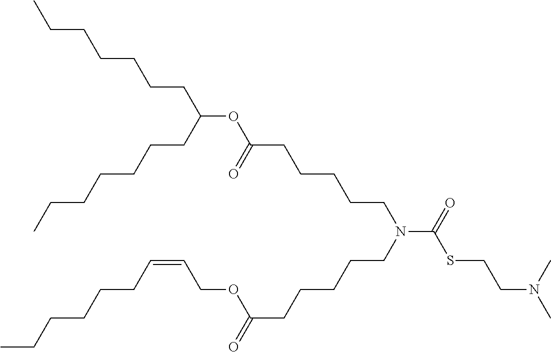 [SMILES: CCCCCC/C=C\COC(=O)CCCCCN(CCCCCC(=O)OC(CCCCCCC)CCCCCCC)C(=O)SCCN(C)C] |
| 4 | 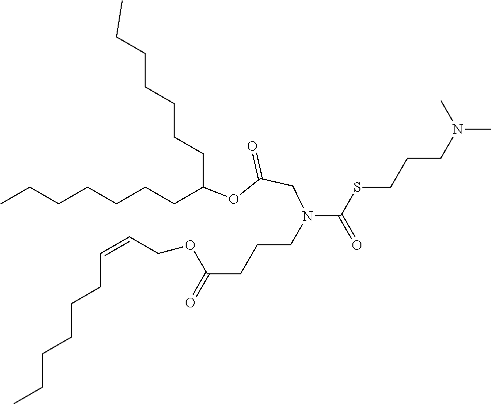 [SMILES: CCCCCC/C=C\COC(=O)CCCN(CC(=O)OC(CCCCCCC)CCCCCCC)C(=O)SCCCN(C)C] |
| 5 | 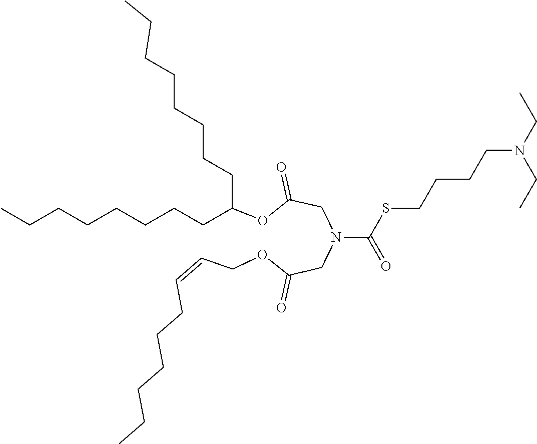 [SMILES: CCCCCC/C=C\COC(=O)CN(CC(=O)OC(CCCCCCCC)CCCCCCCC)C(=O)SCCCCN(CC)CC] |
| 6 | 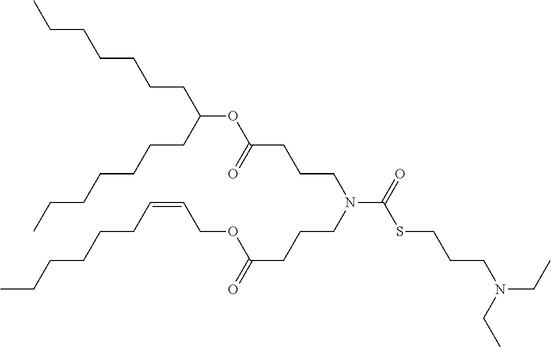 [SMILES: CCCCCC/C=C\COC(=O)CCCN(CCCC(=O)OC(CCCCCCC)CCCCCCC)C(=O)SCCCN(CC)CC] |
| 7 | 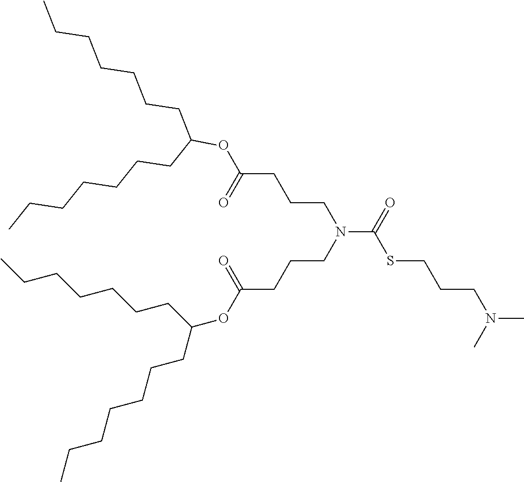 [SMILES: CCCCCCCC(CCCCCCC)OC(=O)CCCN(CCCC(=O)OC(CCCCCCC)CCCCCCC)C(=O)SCCCN(C)C] |
| 8 | 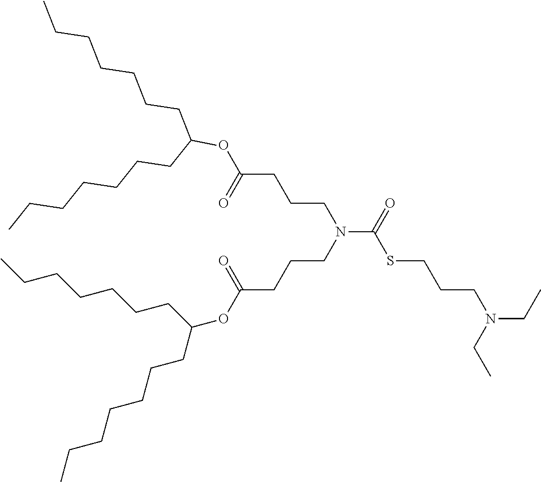 [SMILES: CCCCCCCC(CCCCCCC)OC(=O)CCCN(CCCC(=O)OC(CCCCCCC)CCCCCCC)C(=O)SCCCN(CC)CC] |

In some embodiments, the lipid formulation comprises an ionizable cationic lipid having a structure selected from

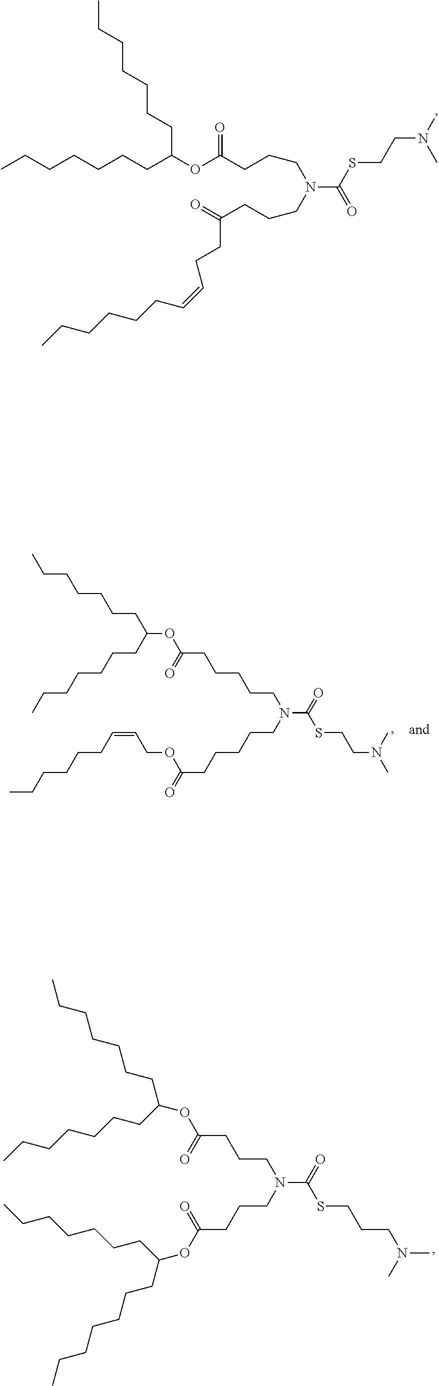

or a pharmaceutically acceptable salt thereof.
In some preferred embodiments, the lipid formulation comprises an ionizable cationic lipid having the structure

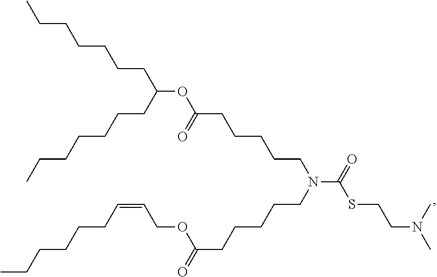 [SMILES: CCCCCC/C=C\COC(=O)CCCCCN(CCCCCC(=O)OC(CCCCCCC)CCCCCCC)C(=O)SCCN(C)C]

or a pharmaceutically acceptable salt thereof.

In embodiments, any one or more lipids recited herein may be expressly excluded.

In some aspects, the helper lipid comprises from about 2 mol % to about 20 mol %, from about 3 mol % to about 18 mol %, from about 4 mol % to about 16 mol %, about 5 mol % to about 14 mol %, from about 6 mol % to about 12 mol %, from about 5 mol % to about 10 mol %, from about 5 mol % to about 9 mol %, or about 2 mol %, about 3 mol %, about 4 mol %, about 5 mol %, about 6 mol %, about 7 mol %, about 8 mol %, about 9 mol %, about 10 mol %, about 11 mol %, or about 12 mol % (or any fraction thereof or the range therein) of the total lipid present in the lipid formulation.

The cholesterol or cholesterol derivative in the lipid formulation may comprise up to about 40 mol %, about 45 mol %, about 50 mol %, about 55 mol %, or about 60 mol % of the total lipid present in the lipid formulation. In some aspects, the cholesterol or cholesterol derivative comprises about 15 mol % to about 45 mol %, about 20 mol % to about 40 mol %, about 25 mol % to about 35 mol %, or about 28 mol % to about 35 mol %; or about 25 mol %, about 26 mol %, about 27 mol %, about 28 mol %, about 29 mol %, about 30 mol %, about 31 mol %, about 32 mol %, about 33 mol %, about 34 mol %, about 35 mol %, about 36 mol %, or about 37 mol % of the total lipid present in the lipid formulation.

In some aspects, the phospholipid component in the mixture may comprise from about 2 mol % to about 20 mol %, from about 3 mol % to about 18 mol %, from about 4 mol % to about 16 mol %, about 5 mol % to about 14 mol %, from about 6 mol % to about 12 mol %, from about 5 mol % to about 10 mol %, from about 5 mol % to about 9 mol %, or about 2 mol %, about 3 mol %, about 4 mol %, about 5 mol %, about 6 mol %, about 7 mol %, about 8 mol %, about 9 mol %, about 10 mol %, about 11 mol %, or about 12 mol % (or any fraction thereof or the range therein) of the total lipid present in the lipid formulation.

The percentage of helper lipid present in the lipid formulation is a target amount, and the actual amount of helper lipid present in the formulation may vary, for example, by ±5 mol %.

A lipid formulation that includes a cationic lipid compound or ionizable cationic lipid compound may be on a molar basis about 30-70% cationic lipid compound, about 25-40% cholesterol, about 2-15% helper lipid, and about 0.5-5% of a polyethylene glycol (PEG) lipid, wherein the percent is of the total lipid present in the formulation. In some aspects, the composition is about 40-65% cationic lipid compound, about 25-35% cholesterol, about 3-9% helper lipid, and about 0.5-3% of a PEG-lipid, wherein the percent is of the total lipid present in the formulation.

The formulation may be a lipid particle formulation, for example containing 8-30% nucleic acid compound, 5-30% helper lipid, and 0-20% cholesterol; 4-25% cationic lipid, 4-25% helper lipid, 2-25% cholesterol, 10-35% cholesterol-PEG, and 5% cholesterol-amine; or 2-30% cationic lipid, 2-30% helper lipid, 1-15% cholesterol, 2-35% cholesterol-PEG, and 1-20% cholesterol-amine; or up to 90% cationic lipid and 2-10% helper lipids, or even 100% cationic lipid.

Lipid Conjugates

The lipid formulations described herein may further comprise a lipid conjugate. The conjugated lipid is useful in that it prevents the aggregation of particles. Suitable conjugated lipids include, but are not limited to, PEG-lipid conjugates, cationic-polymer-lipid conjugates, and mixtures thereof. Furthermore, lipid delivery vehicles can be used for specific targeting by attaching ligands (e.g., antibodies, peptides, and carbohydrates) to its surface or to the terminal end of the attached PEG chains (Front Pharmacol. 2015 Dec. 1; 6:286).

In some aspects, the lipid conjugate is a PEG-lipid. The inclusion of polyethylene glycol (PEG) in a lipid formulation as a coating or surface ligand, a technique referred to as PEGylation, helps to protect nanoparticles from the immune system and their escape from RES uptake (Nanomedicine (Lond). 2011 June; 6(4):715-28). PEGylation has been used to stabilize lipid formulations and their payloads through physical, chemical, and biological mechanisms. Detergent-like PEG lipids (e.g., PEG-DSPE) can enter the lipid formulation to form a hydrated layer and steric barrier on the surface. Based on the degree of PEGylation, the surface layer can be generally divided into two types, brush-like and mushroom-like layers. For PEG-DSPE-stabilized formulations, PEG will take on the mushroom conformation at a low degree of PEGylation (usually less than 5 mol %) and will shift to brush conformation as the content of PEG-DSPE is increased past a certain level (Journal of Nanomaterials. 2011; 2011:12). PEGylation leads to a significant increase in the circulation half-life of lipid formulations (Annu. Rev. Biomed. Eng. 2011 Aug. 15; 130:507-30; J. Control Release. 2010 Aug. 3; 145(3):178-81).

Examples of PEG-lipids include, but are not limited to, PEG coupled to dialkyloxypropyls (PEG-DAA), PEG coupled to diacylglycerol (PEG-DAG), PEG coupled to phospholipids such as phosphatidylethanolamine (PEG-PE), PEG conjugated to ceramides, PEG conjugated to cholesterol or a derivative thereof, and mixtures thereof.

PEG is a linear, water-soluble polymer of ethylene PEG repeating units with two terminal hydroxyl groups. PEGs are classified by their molecular weights and include the following: monomethoxypolyethylene glycol (MePEG-OH), monomethoxypolyethylene glycol-succinate (MePEG-S), monomethoxypolyethylene glycol-succinimidyl succinate (MePEG-S-NHS), monomethoxypolyethylene glycol-amine (MePEG-NH2), monomethoxypolyethylene glycol-tresylate (MePEG-TRES), monomethoxypolyethylene glycol-imidazolyl-carbonyl (MePEG-IM), as well as such compounds containing a terminal hydroxyl group instead of a terminal methoxy group (e.g., HO-PEG-S, HO-PEG-S-NHS, HO-PEG-NH2).

The PEG moiety of the PEG-lipid conjugates described herein may comprise an average molecular weight ranging from about 550 daltons to about 10,000 daltons. In certain aspects, the PEG moiety has an average molecular weight of from about 750 daltons to about 5,000 daltons (e.g., from about 1,000 daltons to about 5,000 daltons, from about 1,500 daltons to about 3,000 daltons, from about 750 daltons to about 3,000 daltons, from about 750 daltons to about 2,000 daltons). In some aspects, the PEG moiety has an average molecular weight of about 2,000 daltons or about 750 daltons. The average molecular weight may be any value or subvalue within the recited ranges, including endpoints.

In certain aspects, the PEG can be optionally substituted by an alkyl, alkoxy, acyl, or aryl group. The PEG can be conjugated directly to the lipid or may be linked to the lipid via a linker moiety. Any linker moiety suitable for coupling the PEG to a lipid can be used including, e.g., non-ester-containing linker moieties and ester-containing linker moieties. In one aspect, the linker moiety is a non-ester-containing linker moiety. Exemplary non-ester-containing linker moieties include, but are not limited to, amido (—C(O)NH—), amino (—NR—), carbonyl (—C(O)—), carbamate (—NHC(O)O—), urea (—NHC(O)NH—), disulfide (—S—S—), ether (—O—), succinyl (—(O)CCH2CH2C(O)—), succinamidyl (—NHC(O)CH2CH2C(O)NH—), ether, as well as combinations thereof (such as a linker containing both a carbamate linker moiety and an amido linker moiety). In one aspect, a carbamate linker is used to couple the PEG to the lipid.

In some aspects, an ester-containing linker moiety is used to couple the PEG to the lipid. Exemplary ester-containing linker moieties include, e.g., carbonate (—OC(O)O—), succinoyl, phosphate esters (—O—(O)POH—O—), sulfonate esters, and combinations thereof.

Phosphatidylethanolamines having a variety of acyl chain groups of varying chain lengths and degrees of saturation can be conjugated to PEG to form the lipid conjugate. Such phosphatidylethanolamines are commercially available or can be isolated or synthesized using conventional techniques known to those of skill in the art. Phosphatidylethanolamines containing saturated or unsaturated fatty acids with carbon chain lengths in the range of C10 to C20 are preferred. Phosphatidylethanolamines with mono- or di-unsaturated fatty acids and mixtures of saturated and unsaturated fatty acids can also be used. Suitable phosphatidylethanolamines include, but are not limited to, dimyristoyl-phosphatidylethanolamine (DMPE), dipalmitoyl-phosphatidylethanolamine (DPPE), dioleoyl-phosphatidylethanolamine (DOPE), and distearoyl-phosphatidylethanolamine (DSPE).

In some aspects, the PEG-DAA conjugate is a PEG-didecyloxypropyl (C10) conjugate, a PEG-dilauryloxypropyl (C12) conjugate, a PEG-dimyristyloxypropyl (C14) conjugate, a PEG-dipalmityloxypropyl (C16) conjugate, or a PEG-distearyloxypropyl (C18) conjugate. In some aspects, the PEG has an average molecular weight of about 750 or about 2,000 daltons. In some aspects, the terminal hydroxyl group of the PEG is substituted with a methyl group.

In addition to the foregoing, other hydrophilic polymers can be used in place of PEG. Examples of suitable polymers that can be used in place of PEG include, but are not limited to, polyvinylpyrrolidone, polymethyloxazoline, polyethyloxazoline, polyhydroxypropyl, methacrylamide, polymethacrylamide, and polydimethylacrylamide, polylactic acid, polyglycolic acid, and derivatized celluloses such as hydroxymethylcellulose or hydroxyethylcellulose.

In some aspects, the lipid conjugate (e.g., PEG-lipid) comprises from about 0.1 mol % to about 2 mol %, from about 0.5 mol % to about 2 mol %, from about 1 mol % to about 2 mol %, from about 0.6 mol % to about 1.9 mol %, from about 0.7 mol % to about 1.8 mol %, from about 0.8 mol % to about 1.7 mol %, from about 0.9 mol % to about 1.6 mol %, from about 0.9 mol % to about 1.8 mol %, from about 1 mol % to about 1.8 mol %, from about 1 mol % to about 1.7 mol %, from about 1.2 mol % to about 1.8 mol %, from about 1.2 mol % to about 1.7 mol %, from about 1.3 mol % to about 1.6 mol %, or from about 1.4 mol % to about 1.6 mol % (or any fraction thereof or range therein) of the total lipid present in the lipid formulation. In other embodiments, the lipid conjugate (e.g., PEG-lipid) comprises about 0.5%, 0.6%, 0.7%, 0.8%, 0.9%, 1.0%, 1.2%, 1.3%, 1.4%, 1.5%, 1.6%, 1.7%, 1.8%, 1.9%, 2.0%, 2.5%, 3.0%, 3.5%, 4.0%, 4.5%, or 5%, (or any fraction thereof or range therein) of the total lipid present in the lipid formulation. The amount may be any value or subvalue within the recited ranges, including endpoints.

The percentage of lipid conjugate (e.g., PEG-lipid) present in the lipid formulations of the disclosure is a target amount, and the actual amount of lipid conjugate present in the formulation may vary, for example, by ±0.5 mol %. One of ordinary skill in the art will appreciate that the concentration of the lipid conjugate can be varied depending on the lipid conjugate employed and the rate at which the lipid formulation is to become fusogenic.

Mechanism of Action for Cellular Uptake of Lipid Formulations

In some aspects, lipid formulations for the intracellular delivery of nucleic acids, particularly liposomes, cationic liposomes, and lipid nanoparticles, are designed for cellular uptake by penetrating target cells through exploitation of the target cells' endocytic mechanisms where the contents of the lipid delivery vehicle are delivered to the cytosol of the target cell. (Nucleic Acid Therapeutics, 28(3):146-157, 2018). Prior to endocytosis, functionalized ligands such as PEG-lipid at the surface of the lipid delivery vehicle are shed from the surface, which triggers internalization into the target cell. During endocytosis, some part of the plasma membrane of the cell surrounds the vector and engulfs it into a vesicle that then pinches off from the cell membrane, enters the cytosol and ultimately enters and moves through the endolysosomal pathway. For ionizable cationic lipid-containing delivery vehicles, the increased acidity as the endosome ages results in a vehicle with a strong positive charge on the surface. Interactions between the delivery vehicle and the endosomal membrane then result in a membrane fusion event that leads to cytosolic delivery of the payload. For RNA payloads, the cell's own internal translation processes will then translate the RNA into the encoded protein. The encoded protein can further undergo postranslational processing, including transportation to a targeted organelle or location within the cell or excretion from the cell.

By controlling the composition and concentration of the lipid conjugate, one can control the rate at which the lipid conjugate exchanges out of the lipid formulation and, in turn, the rate at which the lipid formulation becomes fusogenic. In addition, other variables including, e.g., pH, temperature, or ionic strength, can be used to vary and/or control the rate at which the lipid formulation becomes fusogenic. Other methods which can be used to control the rate at which the lipid formulation becomes fusogenic will become apparent to those of skill in the art upon reading this disclosure. Also, by controlling the composition and concentration of the lipid conjugate, one can control the liposomal or lipid particle size.

Lipid Formulation Manufacture

There are many different methods for the preparation of lipid formulations comprising a nucleic acid. (Curr. Drug Metabol. 2014, 15, 882-892; Chem. Phys. Lipids 2014, 177, 8-18; Int. J. Pharm. Stud. Res. 2012, 3, 14-20). The techniques of thin film hydration, double emulsion, reverse phase evaporation, microfluidic preparation, dual assymetric centrifugation, ethanol injection, detergent dialysis, spontaneous vesicle formation by ethanol dilution, and encapsulation in preformed liposomes are briefly described herein.

Thin Film Hydration

In Thin Film Hydration (TFH) or the Bangham method, the lipids are dissolved in an organic solvent, then evaporated through the use of a rotary evaporator leading to a thin lipid layer formation. After the layer hydration by an aqueous buffer solution containing the compound to be loaded, Multilamellar Vesicles (MLVs) are formed, which can be reduced in size to produce Small or Large Unilamellar vesicles (LUV and SUV) by extrusion through membranes or by the sonication of the starting MLV.

Double Emulsion

Lipid formulations can also be prepared through the Double Emulsion technique, which involves lipids dissolution in a water/organic solvent mixture. The organic solution, containing water droplets, is mixed with an excess of aqueous medium, leading to a water-in-oil-in-water (W/O/W) double emulsion formation. After mechanical vigorous shaking, part of the water droplets collapse, giving Large Unilamellar Vesicles (LUVs).

Reverse Phase Evaporation

The Reverse Phase Evaporation (REV) method also allows one to achieve LUVs loaded with nucleic acid. In this technique a two-phase system is formed by phospholipids dissolution in organic solvents and aqueous buffer. The resulting suspension is then sonicated briefly until the mixture becomes a clear one-phase dispersion. The lipid formulation is achieved after the organic solvent evaporation under reduced pressure. This technique has been used to encapsulate different large and small hydrophilic molecules including nucleic acids.

Microfluidic Preparation

The Microfluidic method, unlike other bulk techniques, gives the possibility of controlling the lipid hydration process. The method can be classified in continuous-flow microfluidic and droplet-based microfluidic, according to the way in which the flow is manipulated. In the microfluidic hydrodynamic focusing (MHF) method, which operates in a continuous flow mode, lipids are dissolved in isopropyl alcohol which is hydrodynamically focused in a microchannel cross junction between two aqueous buffer streams. Vesicles size can be controlled by modulating the flow rates, thus controlling the lipids solution/buffer dilution process. The method can be used for producing oligonucleotide (ON) lipid formulations by using a microfluidic device consisting of three-inlet and one-outlet ports.

Dual Asymmetric Centrifugation

Dual Asymmetric Centrifugation (DAC) differs from more common centrifugation as it uses an additional rotation around its own vertical axis. An efficient homogenization is achieved due to the two overlaying movements generated: the sample is pushed outwards, as in a normal centrifuge, and then it is pushed towards the center of the vial due to the additional rotation. By mixing lipids and an NaCl-solution a viscous vesicular phospholipid gel (VPC) is achieved, which is then diluted to obtain a lipid formulation dispersion. The lipid formulation size can be regulated by optimizing DAC speed, lipid concentration and homogenization time.

Ethanol Injection

The Ethanol Injection (EI) method can be used for nucleic acid encapsulation. This method provides the rapid injection of an ethanolic solution, in which lipids are dissolved, into an aqueous medium containing nucleic acids to be encapsulated, through the use of a needle. Vesicles are spontaneously formed when the phospholipids are dispersed throughout the medium.

Detergent Dialysis

The Detergent dialysis method can be used to encapsulate nucleic acids. Briefly lipid and plasmid are solubilized in a detergent solution of appropriate ionic strength, after removing the detergent by dialysis, a stabilized lipid formulation is formed. Unencapsulated nucleic acid is then removed by ion-exchange chromatography and empty vesicles by sucrose density gradient centrifugation. The technique is highly sensitive to the cationic lipid content and to the salt concentration of the dialysis buffer, and the method is also difficult to scale.

Spontaneous Vesicle Formation by Ethanol Dilution

Stable lipid formulations can also be produced through the Spontaneous Vesicle Formation by Ethanol Dilution method in which a stepwise or dropwise ethanol dilution provides the instantaneous formation of vesicles loaded with nucleic acid by the controlled addition of lipid dissolved in ethanol to a rapidly mixing aqueous buffer containing the nucleic acid.

Encapsulation in Preformed Liposomes

The entrapment of nucleic acids can also be obtained starting with preformed liposomes through two different methods: (1) A simple mixing of cationic liposomes with nucleic acids which gives electrostatic complexes called “lipoplexes”, where they can be successfully used to transfect cell cultures, but are characterized by their low encapsulation efficiency and poor performance in vivo; and (2) a liposomal destabilization, slowly adding absolute ethanol to a suspension of cationic vesicles up to a concentration of 40% v/v followed by the dropwise addition of nucleic acids achieving loaded vesicles; however, the two main steps characterizing the encapsulation process are too sensitive, and the particles have to be downsized.

Excipients

The pharmaceutical compositions disclosed herein can be formulated using one or more excipients to: (1) increase stability; (2) increase cell transfection; (3) permit a sustained or delayed release (e.g., from a depot formulation of the polynucleotide, primary construct, or RNA); (4) alter the biodistribution (e.g., target the polynucleotide, primary construct, or RNA to specific tissues or cell types); (5) increase the translation of encoded protein in vivo; and/or (6) alter the release profile of encoded protein in vivo.

The pharmaceutical compositions described herein may be prepared by any method known or hereafter developed in the art of pharmacology. In general, such preparatory methods include the step of associating the active ingredient (i.e., nucleic acid) with an excipient and/or one or more other accessory ingredients. A pharmaceutical composition in accordance with the present disclosure may be prepared, packaged, and/or sold in bulk, as a single unit dose, and/or as a plurality of single unit doses.

Pharmaceutical compositions may additionally comprise a pharmaceutically acceptable excipient, which, as used herein, includes, but is not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired.

In addition to traditional excipients such as any and all solvents, dispersion media, diluents, or other liquid vehicles, dispersion or suspension aids, surface active agents, isotonic agents, thickening or emulsifying agents, preservatives, excipients of the present disclosure can include, without limitation, liposomes, lipid nanoparticles, polymers, lipoplexes, core-shell nanoparticles, peptides, proteins, cells transfected with primary DNA construct, or RNA (e.g., for transplantation into a subject), hyaluronidase, nanoparticle mimics and combinations thereof.

Accordingly, the pharmaceutical compositions described herein can include one or more excipients, each in an amount that together increases the stability of the nucleic acid in the lipid formulation, increases cell transfection by the nucleic acid, increases the expression of the encoded protein, and/or alters the release profile of encoded proteins. Further, the RNA of the present disclosure may be formulated using self-assembled nucleic acid nanoparticles.

Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, Md., 2006; incorporated herein by reference in its entirety). The use of a conventional excipient medium may be contemplated within the scope of the embodiments of the present disclosure, except insofar as any conventional excipient medium may be incompatible with a substance or its derivatives, such as by producing any undesirable biological effect or otherwise interacting in a deleterious manner with any other component(s) of the pharmaceutical composition.

The pharmaceutical compositions of this disclosure may further contain as pharmaceutically acceptable carriers substances as required to approximate physiological conditions, such as pH adjusting and buffering agents, tonicity adjusting agents, and wetting agents, for example, sodium acetate, sodium lactate, sodium chloride, potassium chloride, calcium chloride, sorbitan monolaurate, triethanolamine oleate, and mixtures thereof. For solid compositions, conventional nontoxic pharmaceutically acceptable carriers can be used which include, for example, pharmaceutical grades of mannitol, lactose, starch, magnesium stearate, sodium saccharin, talcum, cellulose, glucose, sucrose, magnesium carbonate, and the like.

In certain embodiments of the disclosure, the RNA-lipid formulation may be administered in a time release formulation, for example in a composition which includes a slow release polymer. The active agent can be prepared with carriers that will protect against rapid release, for example a controlled release vehicle such as a polymer, microencapsulated delivery system, or a bioadhesive gel. Prolonged delivery of the RNA, in various compositions of the disclosure can be brought about by including in the composition agents that delay absorption, for example, aluminum monostearate hydrogels and gelatin.

Methods of Inducing Immune Responses

Provided herein, in some embodiments, are methods of inducing an immune response in a subject. Any type of immune response can be induced using the methods provided herein, including adaptive and innate immune responses. In one aspect, immune responses induced using the methods provided herein include an antibody response, a cellular immune response, or both an antibody response and a cellular immune response.

Methods of inducing an immune response provided herein include administering to a subject an effective amount of any nucleic acid molecule provided herein. In one aspect, methods of inducing an immune response include administering to a subject an effective amount of any composition comprising a nucleic acid molecule and a lipid provided herein. In another aspect, methods of inducing an immune response include administering to a subject an effective amount of any pharmaceutical composition comprising a nucleic acid molecule and a lipid formulation provided herein. In some aspects, nucleic acid molecules, compositions, and pharmaceutical composition provided here are vaccines that can elicit a protective or a therapeutic immune response, for example.

As used herein, the term “subject” refers to any individual or patient on which the methods disclosed herein are performed. The term “subject” can be used interchangeably with the term “individual” or “patient.” The subject can be a human, although the subject may be an animal, as will be appreciated by those in the art. Thus, other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject. As used herein, the term “effective amount” or “therapeutically effective amount” refers to that amount of a nucleic acid molecule, composition, or pharmaceutical composition described herein that is sufficient to effect the intended application, including but not limited to inducing an immune response and/or disease treatment, as defined herein. The therapeutically effective amount may vary depending upon the intended application (e.g., inducing an immune response, treatment, application in vivo), or the subject or patient and disease condition being treated, e.g., the weight and age of the subject, the species, the severity of the disease condition, the manner of administration and the like, which can readily be determined by one of ordinary skill in the art. The term also applies to a dose that will induce a particular response in a target cell. The specific dose will vary depending on the particular nucleic acid molecule, composition, or pharmaceutical composition chosen, the dosing regimen to be followed, whether it is administered in combination with other compounds, timing of administration, the tissue to which it is administered, and the physical delivery system in which it is carried.

Exemplary doses of nucleic acid molecules that can be administered include about 0.01 μg, about 0.02 μg, about 0.03 μg, about 0.04 μg, about 0.05 μg, about 0.06 μg, about 0.07 μg, about 0.08 μg, about 0.09 μg, about 0.1 μg, about 0.2 μg, about 0.3 μg, about 0.4 μg, about 0.5 μg, about 0.6 μg, about 0.7 μg, about 0.8 μg, about 0.9 μg, about 1.0 μg, about 1.5 μg, about 2.0 μg, about 2.5 μg, about 3.0 μg, about 3.5 μg, about 4.0 μg, about 4.5 μg, about 5.0 μg, about 5.5 μg about 6.0 μg, about 6.5 μg about 7.0 μg, about 7.5 μg, about 8.0 μg, about 8.5 μg, about 9.0 μg, about 9.5 μg, about 10 μg, about 11 μg, about 12 μg, about 13μ, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29 μg, about 30 μg, about 35 μg, about 40 μg, about 45 μg, about 50 μg, about 55 μg, about 60 μg, about 65 μg, about 70 μg, about 75 μg, about 80 μg, about 85 μg, about 90 μg, about 95 μg, about 100 μg, about 125 μg, about 150 μg, about 175 μg, about 200 μg, about 250 μg, about 300 μg, about 350 μg, about 400 μg, about 450 μg, about 500 μg, about 600 μg, about 700 μg, about 800 μg, about 900 μg, about 1,000 μg, or more, and any number or range in between. In one aspect, the nucleic acid molecules are RNA molecules. In another aspect, the nucleic acid molecules are DNA molecules. Nucleic acid molecules can have a unit dosage comprising about 0.01 μg to about 1,000 μg or more nucleic acid in a single dose.

In some aspects, compositions provided herein that can be administered include about 0.01 μg, about 0.02 μg, about 0.03 μg, about 0.04 μg, about 0.05 μg, about 0.06 μg, about 0.07 μg, about 0.08 μg, about 0.09 μg, about 0.1 μg, about 0.2 μg, about 0.3 μg, about 0.4 μg, about 0.5 μg, about 0.6 μg, about 0.7 μg, about 0.8 μg, about 0.9 μg, about 1.0 μg, about 1.5 μg, about 2.0 μg, about 2.5 μg, about 3.0 μg, about 3.5 μg, about 4.0 μg, about 4.5 μg, about 5.0 μg, about 5.5 μg, about 6.0 μg, about 6.5 μg, about 7.0 μg, about 7.5 μg, about 8.0 μg, about 8.5 μg, about 9.0 μg, about 9.5 μg, about 10 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29 μg, about 30 μg, about 35 μg, about 40 μg, about 45 μg, about 50 μg, about 55 μg, about 60 μg, about 65 μg, about 70 μg, about 75 μg, about 80 μg, about 85 μg, about 90 μg, about 95 μg, about 100 μg, about 125 μg, about 150 μg, about 175 μg, about 200 μg, about 250 μg, about 300 μg, about 350 μg, about 400 μg, about 450 μg, about 500 μg, about 600 μg, about 700 μg, about 800 μg, about 900 μg, about 1,000 μg, or more, and any number or range in between, nucleic acid and lipid. In other aspects, pharmaceutical compositions provided herein that can be administered include about 0.01 μg, about 0.02 μg, about 0.03 μg, about 0.04 μg, about 0.05 μg, about 0.06 μg, about 0.07 μg, about 0.08 μg, about 0.09 μg, about 0.1 μg, about 0.2 μg, about 0.3 μg, about 0.4 μg, about 0.5 μg, about 0.6 μg, about 0.7 μg, about 0.8 μg, about 0.9 μg, about 1.0 μg, about 1.5 μg, about 2.0 μg, about 2.5 μg, about 3.0 μg, about 3.5 μg, about 4.0 μg, about 4.5 μg, about 5.0 μg, about 5.5 μg, about 6.0 μg, about 6.5 μg, about 7.0 μg, about 7.5 μg, about 8.0 μg, about 8.5 μg, about 9.0 μg, about 9.5 μg, about 10 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29 μg, about 30 μg, about 35 μg, about 40 μg, about 45 μg, about 50 μg, about 55 μg, about 60 μg, about 65 μg, about 70 μg, about 75 μg, about 80 μg, about 85 μg, about 90 μg, about 95 μg, about 100 μg, about 125 μg, about 150 μg, about 175 μg, about 200 μg, about 250 μg, about 300 μg, about 350 μg, about 400 μg, about 450 μg, about 500 μg, about 600 μg, about 700 μg, about 800 μg, about 900 μg, about 1,000 μg, or more, and any number or range in between, nucleic acid and lipid formulation.

In one aspect, compositions provided herein can have a unit dosage comprising about 0.01 μg to about 1,000 μg or more nucleic acid and lipid in a single dose. In another aspect, pharmaceutical compositions provided herein can have a unit dosage comprising about 0.01 μg to about 1,000 μg or more nucleic acid and lipid formulation in a single dose. A vaccine unit dosage can correspond to the unit dosage of nucleic acid molecules, compositions, or pharmaceutical compositions provided herein and that can be administered to a subject. In one aspect, vaccine compositions of the instant disclosure have a unit dosage comprising about 0.01 μg to about 1,000 μg or more nucleic acid and lipid formulation in a single dose. In another aspect, vaccine compositions of the instant disclosure have a unit dosage comprising about 0.01 μg to about 50 μg nucleic acid and lipid formulation in a single dose. In yet another aspect, vaccine compositions of the instant disclosure have a unit dosage comprising about 0.2 μg to about 20 μg nucleic acid and lipid formulation in a single dose.

A dosage form of the composition of this disclosure can be solid, which can be reconstituted in a liquid prior to administration. The solid can be administered as a powder. The solid can be in the form of a capsule, tablet, or gel. In some embodiments, the pharmaceutical composition comprises a nucleic acid lipid formulation that has been lyophilized.

In a preferred embodiment, the dosage form of the pharmaceutical compositions described herein can be a liquid suspension of self-replicating RNA lipid nanoparticles described herein. In some embodiments, the liquid suspension is in a buffered solution. In some embodiments, the buffered solution comprises a buffer selected from the group consisting of HEPES, MOPS, TES, and TRIS. In some embodiments, the buffer has a pH of about 7.4. In some preferred embodiments, the buffer is HEPES. In some further embodiments, the buffered solution further comprises a cryoprotectant. In some embodiments, the cryoprotectant is selected from a sugar and glycerol or a combination of a sugar and glycerol. In some embodiments, the sugar is a dimeric sugar. In some embodiments, the sugar is sucrose. In some preferred embodiments, the buffer comprises HEPES, sucrose, and glycerol at a pH of 7.4. In some embodiments, the suspension is frozen during storage and thawed prior to administration. In some embodiments, the suspension is frozen at a temperature below about 70° C. In some embodiments, the suspension is diluted with sterile water during intravenous administration. In some embodiments, intravenous administration comprises diluting the suspension with about 2 volumes to about 6 volumes of sterile water. In some embodiments, the suspension comprises about 0.1 mg to about 3.0 mg self-replicating RNA/mL, about 15 mg/mL to about 25 mg/mL of an ionizable cationic lipid, about 0.5 mg/mL to about 2.5 mg/mL of a PEG-lipid, about 1.8 mg/mL to about 3.5 mg/mL of a helper lipid, about 4.5 mg/mL to about 7.5 mg/mL of a cholesterol, about 7 mg/mL to about 15 mg/mL of a buffer, about 2.0 mg/mL to about 4.0 mg/mL of NaCl, about 70 mg/mL to about 110 mg/mL of sucrose, and about 50 mg/mL to about 70 mg/mL of glycerol. In some embodiments, a lyophilized self-replicating RNA-lipid nanoparticle formulation can be resuspended in a buffer as described herein.

In some embodiments, the compositions of the disclosure are administered to a subject such that a self-replicating RNA concentration of at least about 0.05 mg/kg, at least about 0.1 mg/kg, at least about 0.5 mg/kg, at least about 1.0 mg/kg, at least about 2.0 mg/kg, at least about 3.0 mg/kg, at least about 4.0 mg/kg, at least about 5.0 mg/kg of body weight is administered in a single dose or as part of single treatment cycle. In some embodiments, the compositions of the disclosure are administered to a subject such that a total amount of at least about 0.1 mg, at least about 0.5 mg, at least about 1.0 mg, at least about 2.0 mg, at least about 3.0 mg, at least about 4.0 mg, at least about 5.0 mg, at least about 6.0 mg, at least about 7.0 mg, at least about 8.0 mg, at least about 9.0 mg, at least about 10 mg, at least about 15 mg, at least about 20 mg, at least about 25 mg, at least about 30 mg, at least about 35 mg, at least about 40 mg, at least about 45 mg, at least about 50 mg, at least about 55 mg, at least about 60 mg, at least about 65 mg, at least about 70 mg, at least about 75 mg, at least about 80 mg, at least about 85 mg, at least about 90 mg, at least about 95 mg, at least about 100 mg, at least about 105 mg, at least about 110 mg, at least about 115 mg, at least about 120 mg, or at least about 125 mg self-replicating RNA is administered in one or more doses up to a maximum dose of about 300 mg, about 350 mg, about 400 mg, about 450 mg, or about 500 mg self-replicating RNA.

Any route of administration can be included in methods provided herein. In some aspects, nucleic acid molecules, compositions, and pharmaceutical compositions provided herein are administered intramuscularly, subcutaneously, intradermally, transdermally, intranasally, orally, sublingually, intravenously, intraperitoneally, topically, by aerosol, or by a pulmonary route, such as by inhalation or by nebulization, for example. In some embodiments, the pharmaceutical compositions described are administered systemically. Suitable routes of administration include, for example, rectal, vaginal, transmucosal, or intestinal administration; parenteral delivery, including intramedullary injections, as well as intrathecal, direct intraventricular, intravenous, intraperitoneal, or intranasal. In particular embodiments, the intramuscular administration is to a muscle selected from the group consisting of skeletal muscle, smooth muscle and cardiac muscle. In some embodiments, the pharmaceutical composition is administered intravenously.

Pharmaceutical compositions may be administered to any desired tissue. In some embodiments, the self-replicating RNA delivered is expressed in a tissue different from the tissue in which the lipid formulation or pharmaceutical composition was administered. In preferred embodiments, self-replicating RNA is delivered and expressed in the liver.

In other aspects, nucleic acid molecules, compositions, and pharmaceutical compositions provided herein are administered intramuscularly.

In some aspects, the subject in which an immune response is induced is a healthy subject. As used herein, the term “healthy subject” refers to a subject not having a condition or disease, including an infectious disease or cancer, for example, or not having a condition or disease against which an immune response is induced. Accordingly, in some aspects, a nucleic acid molecule, composition, or pharmaceutical composition provided herein is administered prophylactically to prevent an infectious disease or cancer, for example. In other aspects, the subject in which an immune response is induced has cancer. The subject may suffer from any cancer or have any tumor, including solid and liquid tumors. In one aspect, the cancer is kidney cancer, renal cancer, urinary bladder cancer, prostate cancer, uterine cancer, breast cancer, cervical cancer, ovarian cancer, lung cancer, liver cancer, stomach cancer, colon cancer, rectal cancer, oral cavity cancer, pharynx cancer, pancreatic cancer, thyroid cancer, melanoma, skin cancer, head and neck cancer, brain cancer, hematopoietic cancer, leukemia, lymphoma, bone cancer, or sarcoma. Accordingly, a nucleic acid molecule, composition, or pharmaceutical composition provided herein can be administered therapeutically, i.e., to treat a condition or disease, such as cancer, after the onset of the condition or disease.

As used herein, the terms “treat,” “treatment,” “therapy,” “therapeutic,” and the like refer to obtaining a desired pharmacologic and/or physiologic effect, including, but not limited to, alleviating, delaying or slowing the progression, reducing the effects or symptoms, preventing onset, inhibiting, ameliorating the onset of a diseases or disorder, obtaining a beneficial or desired result with respect to a disease, disorder, or medical condition, such as a therapeutic benefit and/or a prophylactic benefit. “Treatment,” as used herein, includes any treatment of a disease in a mammal, particularly in a human, and includes: (a) preventing the disease from occurring in a subject, including a subject which is predisposed to the disease or at risk of acquiring the disease but has not yet been diagnosed as having it; (b) inhibiting the disease, i.e., arresting its development; and (c) relieving the disease, i.e., causing regression of the disease. A therapeutic benefit includes eradication or amelioration of the underlying disorder being treated. Also, a therapeutic benefit is achieved with the eradication or amelioration of one or more of the physiological symptoms associated with the underlying disorder such that an improvement is observed in the subject, notwithstanding that the subject may still be afflicted with the underlying disorder. In some aspects, for prophylactic benefit, treatment or compositions for treatment, including pharmaceutical compositions, are administered to a subject at risk of developing a particular disease, or to a subject reporting one or more of the physiological symptoms of a disease, even though a diagnosis of this disease may not have been made. The methods of the present disclosure may be used with any mammal or other animal. In some aspects, treatment results in a decrease or cessation of symptoms. A prophylactic effect includes delaying or eliminating the appearance of a disease or condition, delaying or eliminating the onset of symptoms of a disease or condition, slowing, halting, or reversing the progression of a disease or condition, or any combination thereof.

Nucleic acid molecules, compositions, and pharmaceutical compositions provided herein can be administered once or multiple times. Accordingly, nucleic acid molecules, compositions, and pharmaceutical compositions provided herein can be administered one, two, three, four, five, six, seven, eight, nine, ten, or more times. Timing between two or more administrations can be one week, two weeks, three weeks, four weeks, five weeks, six weeks, seven weeks, eight weeks, nine weeks, weeks, ten weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, or more weeks, and any number or range in between. In some aspects, timing between two or more administrations is one month, two months, three months, four months, five months, six months, seven months, eight months, nine months, ten months, 11 months, 12 months, 13 months, 14 months, 15 months, 16 months, 17 months, 18 months, 19 months, 20 months, 21 months, 22 months, 23 months, 24 months, or more months, and any number or range in between. In other aspects, timing between two or more administrations can be one year, two years, three years, four years, five years, six years, seven years, eight years, nine years, ten years, or more years, and any number or range in between, Timing between the first and any subsequent administration can be the same or different. In one aspect, nucleic acid molecules, compositions, or pharmaceutical compositions provided herein are administered once.

More than one nucleic acid molecule, composition, or pharmaceutical composition can be administered in the methods provided herein. In one aspect, two or more nucleic acid molecules, compositions, or pharmaceutical compositions provided herein are administered simultaneously. In another aspect, two or more nucleic acid molecules, compositions, or pharmaceutical compositions provided herein are administered sequentially. Simultaneous and sequential administrations can include any number and any combination of nucleic acid molecules, compositions, or pharmaceutical compositions provided herein. Multiple nucleic acid molecules, compositions, or pharmaceutical compositions that are administered together or sequentially can include transgenes encoding different antigenic proteins or fragments thereof. In this manner, immune responses against different antigenic targets can be induced. Two, three, four, five, six, seven, eight, nine, ten, or more nucleic acid molecules, compositions, or pharmaceutical compositions including transgenes encoding different antigenic proteins or fragments thereof can be administered simultaneously or sequentially. Any combination of nucleic acid molecules, compositions, and pharmaceutical compositions including any combination of transgenes can be administered simultaneously or sequentially. In some aspects, administration is simultaneous. In other aspects, administration is sequential. Timing between two or more administrations can be one week, two weeks, three weeks, four weeks, five weeks, six weeks, seven weeks, eight weeks, nine weeks, weeks, ten weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, or more weeks, and any number or range in between. In some aspects, timing between two or more administrations is one month, two months, three months, four months, five months, six months, seven months, eight months, nine months, ten months, 11 months, 12 months, 13 months, 14 months, 15 months, months, 16 months, 17 months, 18 months, 19 months, 20 months, 21 months, 22 months, 23 months, 24 months, or more months, and any number or range in between. In other aspects, timing between two or more administrations can be one year, two years, three years, four years, five years, six years, seven years, eight years, nine years, ten years, or more years, and any number or range in between, Timing between the first and any subsequent administration can be the same or different. Nucleic acid molecules, compositions, and pharmaceutical compositions provided herein can be administered with any other vaccine or treatment.

Following administration of the composition to the subject, the protein product encoded by the self-replicating RNA of the disclosure (e.g., an antigen) is detectable in the target tissues for at least about one to seven days or longer. For example, the protein product may be detectable in the target tissues at a concentration (e.g., a therapeutic concentration) of at least about 0.025-1.5 μg/ml (e.g., at least about 0.050 μg/ml, at least about 0.075 μg/ml, at least about 0.1 μg/ml, at least about 0.2 μg/ml, at least about 0.3 μg/ml, at least about 0.4 μg/ml, at least about 0.5 μg/ml, at least about 0.6 μg/ml, at least about 0.7 μg/ml, at least about 0.8 μg/ml, at least about 0.9 μg/ml, at least about 1.0 μg/ml, at least about 1.1 μg/ml, at least about 1.2 μg/ml, at least about 1.3 μg/ml, at least about 1.4 μg/ml, or at least about 1.5 μg/ml), for at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45 days or longer following administration of the composition to the subject.

In some embodiments, a pharmaceutical composition of the present disclosure is administered to a subject once per month. In some embodiments, a pharmaceutical composition of the present disclosure is administered to a subject twice per month. In some embodiments, a pharmaceutical composition of the present disclosure is administered to a subject three times per month. In some embodiments, a pharmaceutical composition of the present disclosure is administered to a subject four times per month.

Alternatively, the compositions of the present disclosure may be administered in a local rather than systemic manner, for example, via injection of the pharmaceutical composition directly into a targeted tissue, preferably in a depot or sustained release formulation. Local delivery can be affected in various ways, depending on the tissue to be targeted. For example, aerosols containing compositions of the present disclosure can be inhaled (for nasal, tracheal, or bronchial delivery); compositions of the present disclosure can be injected into the site of injury, disease manifestation, or pain, for example; compositions can be provided in lozenges for oral, tracheal, or esophageal application; can be supplied in liquid, tablet or capsule form for administration to the stomach or intestines, can be supplied in suppository form for rectal or vaginal application; or can even be delivered to the eye by use of creams, drops, or even injection. Formulations containing compositions of the present disclosure complexed with therapeutic molecules or ligands can even be surgically administered, for example in association with a polymer or other structure or substance that can allow the compositions to diffuse from the site of implantation to surrounding cells. Alternatively, they can be applied surgically without the use of polymers or supports.

The self-replicating RNA, formulations thereof, or encoded proteins described herein may be used in combination with one or more other therapeutic, prophylactic, diagnostic, or imaging agents. By “in combination with,” it is not intended to imply that the agents must be administered at the same time and/or formulated for delivery together, although these methods of delivery are within the scope of the present disclosure. Compositions can be administered concurrently with, prior to, or subsequent to, one or more other desired therapeutics or medical procedures. In general, each agent will be administered at a dose and/or on a time schedule determined for that agent. Preferably, the methods of treatment of the present disclosure encompass the delivery of pharmaceutical, prophylactic, diagnostic, or imaging compositions in combination with agents that may improve their bioavailability, reduce and/or modify their metabolism, inhibit their excretion, and/or modify their distribution within the body. As a non-limiting example, a self-replicating RNA of the disclosure may be used in combination with a pharmaceutical agent for immunizing or vaccinating a subject. In general, it is expected that agents utilized in combination with the presently disclosed self-replicating RNA and formulations thereof be utilized at levels that do not exceed the levels at which they are utilized individually. In some embodiments, the levels utilized in combination will be lower than those utilized individually. In one embodiment, the combinations, each or together may be administered according to the split dosing regimens as are known in the art.

Ranges: throughout this disclosure, various aspects can be presented in range format. It should be understood that any description in range format is merely for convenience and brevity and not meant to be limiting. Accordingly, the description of a range should be considered to have specifically disclosed all possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example 1, 2, 2.1, 2.2, 2.5, 3, 4, 4.75, 4.8, 4.85, 4.95, 5, 5.5, 5.75, 5.9, 5.00, and 6. This applies to a range of any breadth.

### Example 1

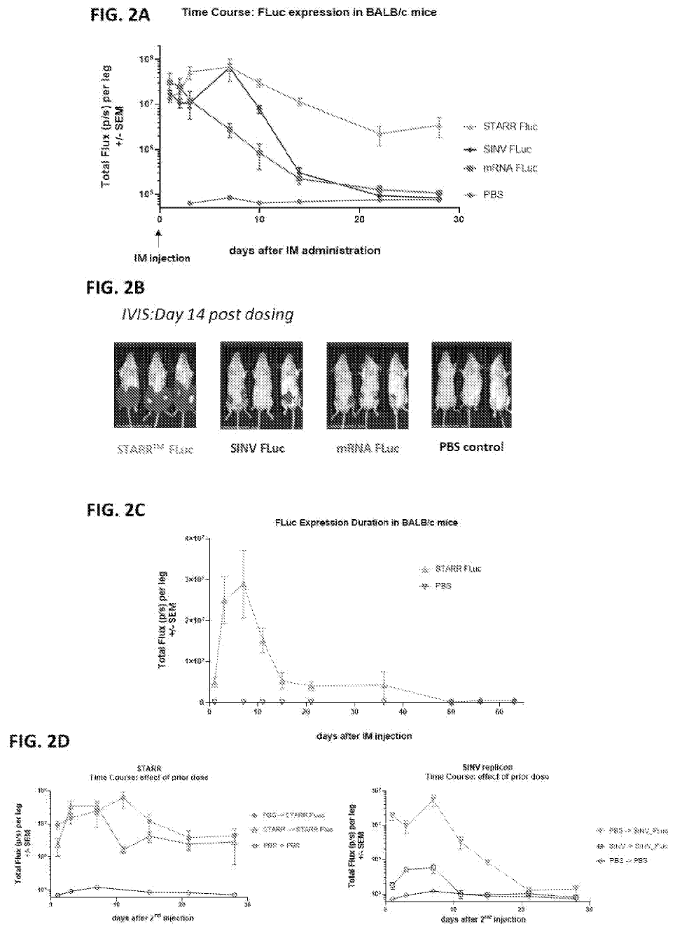

This example describes characterization of self-replicating (STARR™) technology using firefly luciferase transgene expression. In vitro transcripts were formulated with lipid nanoparticles (LNP) at a concentration of 0.1 mg/ml, and injected intramuscularly in both legs of female BALB/C mice (n=3) at a dose of 5 ug per leg. Expression of firefly luciferase (FLuc) was measured by IVIS Lumina LT Series III (PerkinElmer) by administering 100 ul of 1.5 mg Xenolight D-luciferin (PerkinElmer) in PBS via intraperitoneal injection ˜10 min prior to the measurement. Six data points per group of mice were obtained at each time point (FIGS. 2A-2D).

Firefly luciferase (FLuc) expression was monitored from STARR™ Fluc, SINV FLuc, and mRNA FLuc up to day 28 by In Vivo Imaging System (IVIS). Enhanced levels and durations of transgene expression from STARR™ were observed. The expression from STARR™ Fluc peaked around day 3 to 7 and declined until day 22. Fluc expression from SINV FLuc also peaked on day 10, however, the expression was reduced at a significantly faster rate than STARR™ FLuc. Additionally, the expression on day 3 was significantly lower than STARR™ FLuc. FLuc expression from the conventional mRNA backbone was highest at day 1, the earliest time point in this study, and declined at a slightly faster rate than that of STARR™—Fluc (FIG. 2A). FIG. 2B shows that at 14 days post dosing, FLuc expression from STARR™ FLuc was higher than the other groups by about two orders of magnitude. FIG. 2D shows that the effect of the STARR™ backbone remained minimal throughout the experimental period (up to day 28), while prior administration of STNV replicon backbone resulted in a reduction of FLuc transgene expression by ˜2 orders of magnitude.

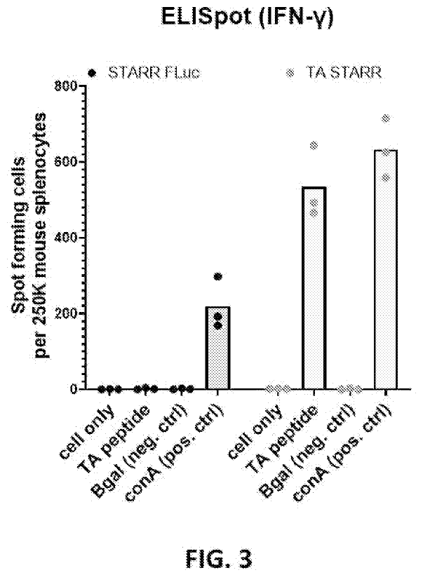

A cancer vaccine substrate, TA STARR™, was constructed next with the STARR™ backbone that encodes AH1A5 epitope from gp70, an envelope glycoprotein of endogenous Murine leukemia virus. AH1 (SPSYVYHQF) (SEQ ID NO:110) is an H-2Ld-restricted antigen of gp70423-431, which is expressed in tumor cells such as the CT26 colorectal cancer cell line, but not expressed in most of the normal tissues. AH1-A5 is a mutated sequence with SPSYAYHQF (SEQ ID NO:111) (the mutation underlined) with enhanced affinity to the T cell receptor (Slansky, et al., 2000, Immunity 13: 529-538). The open reading frame of the TA STARR™ subgenomic RNA contains a cassette with a signal peptide from the HLA class I antigen, gp70 sequence containing AH1A5 epitope, ovalbumin epitope (OVA323-339), and MHC class I trafficking signal (Kreiter, et al. 2008, J Immunol 180: 309-318). Three female BALB/c mice were intramuscularly injected with 10 ug of LNP formulated STARR™ transcripts, STARR™ FLuc or TA STARR™, on day 0 and day 7. On day 16, the spleens were harvested and the splenocytes were isolated. Splenocytes (2.5×10⁵ cells) were incubated with or without AH1A5 (SPSYAYHQF) (SEQ ID NO:111), beta-gal peptide (TPHPARIGL) (SEQ ID NO:112) at 1 ug/ml, and 1× Concanavalin A (Life Technologies). ELISpot detecting murine IFN-gamma (ImmunoSpot) was performed according to the manufacturer's instructions. As can be seen in FIG. 3, TA STARR™ elicited antigen-specific IFN-gamma responses.

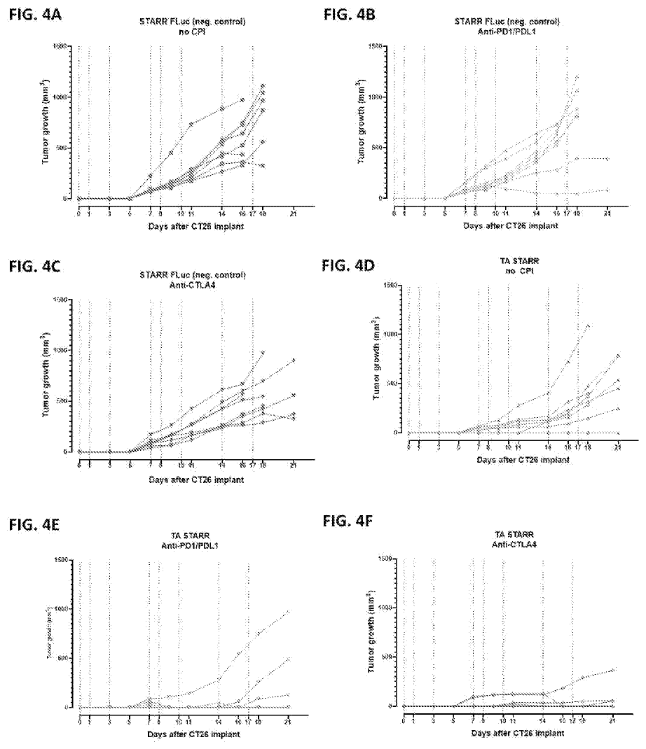

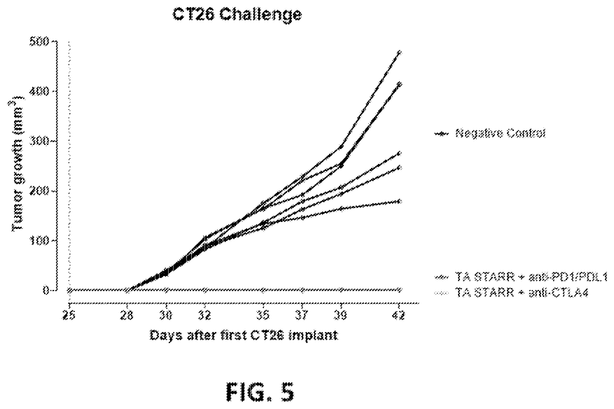

BALB/c mice, 10 week-old female, were subcutaneously implanted in the right flank with 5×10⁵ cells of CT26 cells in PBS. A day later, LNP formulated STARR™ RNA was injected intramuscularly in the left leg at a dose of 10 ug in 100 ul. The mice were administered another booster shot on day 8 with the same dose. For a group with combination treatment of anti-mouse PD1 (RMP1-14, BioXCell) and anti-mouse PDL1 (10F.9G2, BioXcell), the combined checkpoint inhibitor (100 ug each) was administered via intraperitoneal injection in the right quadrant twice weekly for two weeks starting on day 3. For a group with the treatment of anti-mouse CTLA4 (9H10, BioxCell), 200 ug of the checkpoint inhibitor was administered in the same manner but starting on day 7. Five mice of the group with the combo treatment of TA STARR™ vaccine and the checkpoint inhibitors remained tumor-free on day 25, and were further challenged by subcutaneous implantation of CT26 (5×105 cells) in the right flank where the implantation site was slightly above the first implantation site. Naïve mice were used as a control group. The tumor growth was monitored for another 17 days (i.e. up to day 42 since the first CT26 implantation) before euthanization. FIGS. 4A-4F illustrate reduced tumor growth resulting from TA STARR™ vaccination and FIG. 5 shows prolonged protection resulting from treatment with the TA STARR™ vaccine in combination with checkpoint inhibitors.

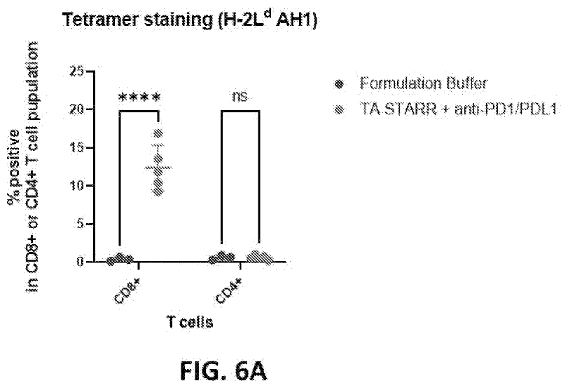

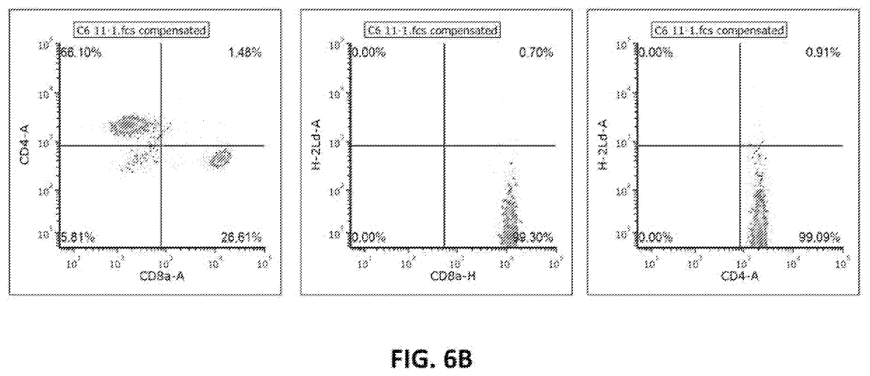

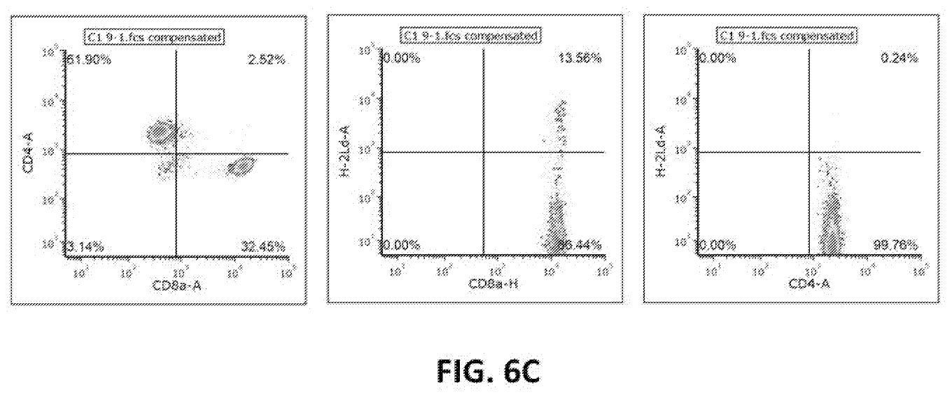

Splenocytes from the combination treatment group with TA STARR™ and anti-PD1/PDL1 were harvested for tetramer staining with AH1 peptide. Splenocytes from the control group with the LNP formulation buffer with the same dosing schedule were used as a negative control. The splenocytes (2×10⁶ cells) were incubated with AH1 (H-2Ld)-tetramer (MBL) followed by appropriate fluorescent-labeled antibodies (Alexa Fluor 488 anti-CD8a (53-6.7), Pacific Orange anti-CD4 (RM4-5), and Pacific Blue anti-mouse CD3E (145-2C11), (eBioscience) and DRAQ7 (Invitrogen) by following the manufacture's recommendation, and 500 K events were analyzed by ZE5 Cell Analyzer (Bio-Rad). Results are shown in FIGS. 6A-6C.

TABLE 6
Transgene ORF nucleotide sequence

| | RNA | | |
|---|---|---|---|
| mARM | back | | |
| # | bone | transgene | Sequence |
| 2809 | STARR^{TM} | Fluc | AUGGAAGAUGCCAAAAACAUUAAGAAGGGCCCAGCGCCAUUCUACC |
| (SEQ | | | CACUCGAAGACGGGACCGCCGGCGAGCAGCUGCACAAAGCCAUGAA |
| ID | | | GCGCUACGCCCUGGUGCCCGGCACCAUCGCCUUUACCGACGCACAU |
| NO: 84) | | | AUCGAGGUGGACAUUACCUACGCCGAGUACUUCGAGAUGAGCGUUC |
| | | | GGCUGGCAGAAGCUAUGAAGCGCUAUGGGCUGAAUACAAACCAUCG |
| | | | GAUCGUGGUGUGCAGCGAGAAUAGCUUGCAGUUCUUCAUGCCCGUG |
| | | | UUGGGUGCCCUGUUCAUCGGUGUGGCUGUGGCCCCAGCUAACGACA |
| | | | UCUACAACGAGCGCGAGCUGCUGAACAGCAUGGGCAUCAGCCAGCC |
| | | | CACCGUCGUAUUCGUGAGCAAGAAAGGGCUGCAAAAGAUCCUCAAC |
| | | | GUGCAAAAGAAGCUACCGAUCAUACAAAAGAUCAUCAUCAUGGAUA |
| | | | GCAAGACCGACUACCAGGGCUUCCAAAGCAUGUACACCUUCGUGAC |
| | | | UUCCCAUUUGCCACCCGGCUUCAACGAGUACGACUUCGUGCCCGAG |
| | | | AGCUUCGACCGGGACAAAACCAUCGCCCUGAUCAUGAACAGUAGUG |
| | | | GCAGUACCGGAUUGCCCAAGGGCGUAGCCCUACCGCACCGCACCGC |
| | | | UUGUGUCCGAUUCAGUCAUGCCCGCGACCCCAUCUUCGGCAACCAG |
| | | | AUCAUCCCCGACACCGCUAUCCUCAGCGUGGUGCCAUUUCACCACG |
| | | | GCUUCGGCAUGUUCACCACGCUGGGCUACUUGAUCUGCGGCUUUCG |
| | | | GGUCGUGCUCAUGUACCGCUUCGAGGAGGAGCUAUUCUUGCGCAGC |
| | | | UUGCAAGACUAUAAGAUUCAAUCUGCCCUGCUGGUGCCCACACUAU |
| | | | UUAGCUUCUUCGCUAAGAGCACUCUCAUCGACAAGUACGACCUAAG |
| | | | CAACUUGCACGAGAUCGCCAGCGGCGGGGCGCCGCUCAGCAAGGAG |
| | | | GUAGGUGAGGCCGUGGCCAAACGCUUCCACCUACCAGGCAUCCGAC |
| | | | AGGGCUACGGCCUGACAGAAACAACCAGCGCCAUUCUGAUCACCCC |
| | | | CGAAGGGGACGACAAGCCUGGCGCAGUAGGCAAGGUGGUGCCCUUC |
| | | | UUCGAGGCUAAGGUGGUGGACUUGGACACCGGUAAGACACUGGGUG |
| | | | UGAACCAGCGCGGCGAGCUGUGCGUCCGUGGCCCCAUGAUCAUGAG |
| | | | CGGCUACGUUAACAACCCCGAGGCUACAAACGCUCUCAUCGACAAG |
| | | | GACGGCUGGCUGCACAGCGGCGACAUCGCCUACUGGGACGAGGACG |
| | | | AGCACUUCUUCAUCGUGGACCGGCUGAAGUCCCUGAUCAAAUACAA |
| | | | GGGCUACCAGGUAGCCCCAGCCGAACUGGAGAGCAUCCUGCUGCAA |
| | | | CACCCCAACAUCUUCGACGCCGGGGUCGCCGGCCUGCCCGACGACG |
| | | | AUGCCGGCGAGCUGCCCGCCGCAGUCGUCGUGCUGGAACACGGUAA |
| | | | AACCAUGACCGAGAAGGAGAUCGUGGACUAUGUGGCCAGCCAGGUU |
| | | | ACAACCGCCAAGAAGCUGCGCGGUGGUGUUGUGUUCGUGGACGAGG |
| | | | UGCCUAAAGGACUGACCGGCAAGUUGGACGCCCGCAAGAUCCGCGA |
| | | | GAUUCUCAUUAAGGCCAAGAAGGGCGGCAAGAUCGCCGUGUAA |
| 2842 | SINV | Fluc | AUGGAAGAUGCCAAAAACAUUAAGAAGGGCCCAGCGCCAUUCUACC |
| (SEQ | replicon | | CACUCGAAGACGGGACCGCCGGCGAGCAGCUGCACAAAGCCAUGAA |
| ID | | | GCGCUACGCCCUGGUGCCCGGCACCAUCGCCUUUACCGACGCACAU |
| NO: 85) | | | AUCGAGGUGGACAUUACCUACGCCGAGUACUUCGAGAUGAGCGUUC |
| | | | GGCUGGCAGAAGCUAUGAAGCGCUAUGGGCUGAAUACAAACCAUCG |
| | | | GAUCGUGGUGUGCAGCGAGAAUAGCUUGCAGUUCUUCAUGCCCGUG |
| | | | UUGGGUGCCCUGUUCAUCGGUGUGGCUGUGGCCCCAGCUAACGACA |
| | | | UCUACAACGAGCGCGAGCUGCUGAACAGCAUGGGCAUCAGCCAGCC |
| | | | CACCGUCGUAUUCGUGAGCAAGAAAGGGCUGCAAAAGAUCCUCAAC |
| | | | GUGCAAAAGAAGCUACCGAUCAUACAAAAGAUCAUCAUCAUGGAUA |
| | | | GCAAGACCGACUACCAGGGCUUCCAAAGCAUGUACACCUUCGUGAC |
| | | | UUCCCAUUUGCCACCCGGCUUCAACGAGUACGACUUCGUGCCCGAG |
| | | | AGCUUCGACCGGGACAAAACCAUCGCCCUGAUCAUGAACAGUAGUG |
| | | | GCAGUACCGGAUUGCCCAAGGGCGUAGCCCUACCGCACCGCACCGC |
| | | | UUGUGUCCGAUUCAGUCAUGCCCGCGACCCCAUCUUCGGCAACCAG |
| | | | AUCAUCCCCGACACCGCUAUCCUCAGCGUGGUGCCAUUUCACCACG |
| | | | GCUUCGGCAUGUUCACCACGCUGGGCUACUUGAUCUGCGGCUUUCG |
| | | | GGUCGUGCUCAUGUACCGCUUCGAGGAGGAGCUAUUCUUGCGCAGC |
| | | | UUGCAAGACUAUAAGAUUCAAUCUGCCCUGCUGGUGCCCACACUAU |
| | | | UUAGCUUCUUCGCUAAGAGCACUCUCAUCGACAAGUACGACCUAAG |
| | | | CAACUUGCACGAGAUCGCCAGCGGCGGGGCGCCGCUCAGCAAGGAG |
| | | | GUAGGUGAGGCCGUGGCCAAACGCUUCCACCUACCAGGCAUCCGAC |
| | | | AGGGCUACGGCCUGACAGAAACAACCAGCGCCAUUCUGAUCACCCC |
| | | | CGAAGGGGACGACAAGCCUGGCGCAGUAGGCAAGGUGGUGCCCUUC |
| | | | UUCGAGGCUAAGGUGGUGGACUUGGACACCGGUAAGACACUGGGUG |
| | | | UGAACCAGCGCGGCGAGCUGUGCGUCCGUGGCCCCAUGAUCAUGAG |
| | | | CGGCUACGUUAACAACCCCGAGGCUACAAACGCUCUCAUCGACAAG |
| | | | GACGGCUGGCUGCACAGCGGCGACAUCGCCUACUGGGACGAGGACG |
| | | | AGCACUUCUUCAUCGUGGACCGGCUGAAGUCCCUGAUCAAAUACAA |
| | | | GGGCUACCAGGUAGCCCCAGCCGAACUGGAGAGCAUCCUGCUGCAA |
| | | | CACCCCAACAUCUUCGACGCCGGGGUCGCCGGCCUGCCCGACGACG |
| | | | AUGCCGGCGAGCUGCCCGCCGCAGUCGUCGUGCUGGAACACGGUAA |
| | | | AACCAUGACCGAGAAGGAGAUCGUGGACUAUGUGGCCAGCCAGGUU |
| | | | ACAACCGCCAAGAAGCUGCGCGGUGGUGUUGUGUUCGUGGACGAGG |
| | | | UGCCUAAAGGACUGACCGGCAAGUUGGACGCCCGCAAGAUCCGCGA |
| | | | GAUUCUCAUUAAGGCCAAGAAGGGCGGCAAGAUCGCCGUGUAA |
| 1782 | mRNA | Fluc | AUGGAAGAUGCCAAAAACAUUAAGAAGGGCCCAGCGCCAUUCUACC |
| (SEQ | (TEV- | | CACUCGAAGACGGGACCGCCGGCGAGCAGCUGCACAAAGCCAUGAA |
| ID | XbG) | | GCGCUACGCCCUGGUGCCCGGCACCAUCGCCUUUACCGACGCACAU |
| NO: 86) | | | AUCGAGGUGGACAUUACCUACGCCGAGUACUUCGAGAUGAGCGUUC |
| | | | GGCUGGCAGAAGCUAUGAAGCGCUAUGGGCUGAAUACAAACCAUCG |
| | | | GAUCGUGGUGUGCAGCGAGAAUAGCUUGCAGUUCUUCAUGCCCGUG |
| | | | UUGGGUGCCCUGUUCAUCGGUGUGGCUGUGGCCCCAGCUAACGACA |
| | | | UCUACAACGAGCGCGAGCUGCUGAACAGCAUGGGCAUCAGCCAGCC |
| | | | CACCGUCGUAUUCGUGAGCAAGAAAGGGCUGCAAAAGAUCCUCAAC |
| | | | GUGCAAAAGAAGCUACCGAUCAUACAAAAGAUCAUCAUCAUGGAUA |
| | | | GCAAGACCGACUACCAGGGCUUCCAAAGCAUGUACACCUUCGUGAC |
| | | | UUCCCAUUUGCCACCCGGCUUCAACGAGUACGACUUCGUGCCCGAG |
| | | | AGCUUCGACCGGGACAAAACCAUCGCCCUGAUCAUGAACAGUAGUG |
| | | | GCAGUACCGGAUUGCCCAAGGGCGUAGCCCUACCGCACCGCACCGC |
| | | | UUGUGUCCGAUUCAGUCAUGCCCGCGACCCCAUCUUCGGCAACCAG |
| | | | AUCAUCCCCGACACCGCUAUCCUCAGCGUGGUGCCAUUUCACCACG |
| | | | GCUUCGGCAUGUUCACCACGCUGGGCUACUUGAUCUGCGGCUUUCG |
| | | | GGUCGUGCUCAUGUACCGCUUCGAGGAGGAGCUAUUCUUGCGCAGC |
| | | | UUGCAAGACUAUAAGAUUCAAUCUGCCCUGCUGGUGCCCACACUAU |
| | | | UUAGCUUCUUCGCUAAGAGCACUCUCAUCGACAAGUACGACCUAAG |
| | | | CAACUUGCACGAGAUCGCCAGCGGCGGGGCGCCGCUCAGCAAGGAG |
| | | | GUAGGUGAGGCCGUGGCCAAACGCUUCCACCUACCAGGCAUCCGAC |
| | | | AGGGCUACGGCCUGACAGAAACAACCAGCGCCAUUCUGAUCACCCC |
| | | | CGAAGGGGACGACAAGCCUGGCGCAGUAGGCAAGGUGGUGCCCUUC |
| | | | UUCGAGGCUAAGGUGGUGGACUUGGACACCGGUAAGACACUGGGUG |
| | | | UGAACCAGCGCGGCGAGCUGUGCGUCCGUGGCCCCAUGAUCAUGAG |
| | | | CGGCUACGUUAACAACCCCGAGGCUACAAACGCUCUCAUCGACAAG |
| | | | GACGGCUGGCUGCACAGCGGCGACAUCGCCUACUGGGACGAGGACG |
| | | | AGCACUUCUUCAUCGUGGACCGGCUGAAGUCCCUGAUCAAAUACAA |
| | | | GGGCUACCAGGUAGCCCCAGCCGAACUGGAGAGCAUCCUGCUGCAA |
| | | | CACCCCAACAUCUUCGACGCCGGGGUCGCCGGCCUGCCCGACGACG |
| | | | AUGCCGGCGAGCUGCCCGCCGCAGUCGUCGUGCUGGAACACGGUAA |
| | | | AACCAUGACCGAGAAGGAGAUCGUGGACUAUGUGGCCAGCCAGGUU |
| | | | ACAACCGCCAAGAAGCUGCGCGGUGGUGUUGUGUUCGUGGACGAGG |
| | | | UGCCUAAAGGACUGACCGGCAAGUUGGACGCCCGCAAGAUCCGCGA |
| | | | GAUUCUCAUUAAGGCCAAGAAGGGCGGCAAGAUCGCCGUGUAA |
| 2847 | STARR^{TM} | KRAS | AUGAAGUUGGUGGUUGUGGGGGCCGGGGGUGUUGGCAAAAGCGCCC |
| (SEQ ID | | epitope wt | UUACAAUUUGA |
| NO: 87) | | | |
| 2862 | SINV | Empty | AUGGAUCCUAGACGCUACGCCCCAAUGAUCCGACCAGCAAAACUCG |
| (SEQ ID | replicon | | AUGUACUUCCGAGGAACUGA |
| NO: 88) | | | |
| 3060 | STARR^{TM} | Signal | AUGAGAGUGACAGCCCCUAGAACCUUACUGCUUCUGCUUUGGGGAG |
| (SEQ | | peptide- | CUGUUGCUCUGACAGAGACAUGGGCUGGAUCUCUGAGCGAGGUGAC |
| ID | | gp70 with | CGGCCAGGGCCUGUGCAUCGGCGCCGUGCCCAAGACCCACCAGGUG |
| NO: 89) | | AH1A5- | CUGUGCAACACCACCCAGAAGACCAGCGACGGCAGCUACUACCUGG |
| | | MITD | CCGCUCCCACCGGCACCACCUGGGCCUGCAGCACCGGCCUGACCCC |
| | | | UUGCAUCAGCACCACCAUCCUGAACCUGACCACCGACUACUGCGUG |
| | | | CUGGUGGAGCUGUGGCCCAGGGUGACCUACCACAGCCCCAGCUACG |
| | | | CCUACCACCAGUUCGAGAGGAGGGCCAAGUACAAGAGGGAGCCCGU |
| | | | GAGCCUGACCCUGGCCCUGCUGCUGGGCGGCCUGACAAUGGGCGGC |
| | | | AUCGCCGCCGGCGUGGGCACCGGCACCACCGCCCUGGUGGCCACCC |
| | | | AGCAGUUCCAGCAGCUGCAGGCCGCCAUGCACGACGACCUGAAGGA |
| | | | GGUGGAGAAGUCCAUCACCAACCUGGAGAAGUCCCUGACCAGCCUG |
| | | | AGCGAGGUGGUGCUGCAGAACAGGAGGGGCCUGGACCUGCUGUUCC |
| | | | UGAAGGAGGGCGGCCUGUGCGCCGCCCUGAAGGAGGAGUGCUGCCU |
| | | | GUACGCCGACCACACCGGCCUGGUGAUCGUGGGCAUUGUCGCUGGC |
| | | | CUGGCCGUCCUCGCCGUGGUGGUGAUUGGAGCUGUGGUCGCAGCUG |
| | | | UUAUGUGCAGAAGAAAGUCAUCCGGCGGAAAGGGAGGCUCCUACUC |
| | | | UCAGGCUGCUUCUGCUACAGUGCCUAGAGCUCUUAUGUGUUUAUCU |
| | | | CAGCUGUAA |
| 3061 | STARR^{TM} | Signal | AUGAGAGUGACAGCCCCUAGAACCUUACUGCUUCUGCUUUGGGGAG |
| (SEQ | | peptide- | CUGUUGCUCUGACAGAGACAUGGGCUGGAUCUUACCACAGCCCCAG |
| ID | | AH1A5 OVA- | CUACGCCUACCACCAGUUCGAGAGGGGGGGAGGAGGCUCCGGGGGA |
| NO: 90) | | MITD | GGAGGCUCCCUGAAGAUCAGCCAGGCCGUGCACGCCGCCCACGCCG |
| | | | AGAUCAACGAGGCCGGCCGGGAGGUGAUCGUGGGCAUUGUCGCUGG |
| | | | CCUGGCCGUCCUCGCCGUGGUGGUGAUUGGAGCUGUGGUCGCAGCU |
| | | | GUUAUGUGCAGAAGAAAGUCAUCCGGCGGAAAGGGAGGCUCCUACU |
| | | | CUCAGGCUGCUUCUGCUACAGUGCCUAGAGCUCUUAUGUGUUUAUC |
| | | | UCAGCUGUAA |
| 3076 | STARR^{TM} | Signal | AUGAGAGUGACAGCCCCUAGAACCUUACUGCUUCUGCUUUGGGGAG |
| (SEQ | | peptide- | CUGUUGCUCUGACAGAGACAUGGGCUGGAUCUCUGAGCGAGGUGAC |
| ID | | gp70 with | CGGCCAGGGCCUGUGCAUCGGCGCCGUGCCCAAGACCCACCAGGUG |
| NO: 91) | | AH1A5- | CUGUGCAACACCACCCAGAAGACCAGCGACGGCAGCUACUACCUGG |
| | | MITD-FLAG | CCGCUCCCACCGGCACCACCUGGGCCUGCAGCACCGGCCUGACCCC |
| | | | UUGCAUCAGCACCACCAUCCUGAACCUGACCACCGACUACUGCGUG |
| | | | CUGGUGGAGCUGUGGCCCAGGGUGACCUACCACAGCCCCAGCUACG |
| | | | CCUACCACCAGUUCGAGAGGAGGGCCAAGUACAAGAGGGAGCCCGU |
| | | | GAGCCUGACCCUGGCCCUGCUGCUGGGCGGCCUGACAAUGGGCGGC |
| | | | AUCGCCGCCGGCGUGGGCACCGGCACCACCGCCCUGGUGGCCACCC |
| | | | AGCAGUUCCAGCAGCUGCAGGCCGCCAUGCACGACGACCUGAAGGA |
| | | | GGUGGAGAAGUCCAUCACCAACCUGGAGAAGUCCCUGACCAGCCUG |
| | | | AGCGAGGUGGUGCUGCAGAACAGGAGGGGCCUGGACCUGCUGUUCC |
| | | | UGAAGGAGGGCGGCCUGUGCGCCGCCCUGAAGGAGGAGUGCUGCCU |
| | | | GUACGCCGACCACACCGGCCUGGUGAUCGUGGGCAUUGUCGCUGGC |
| | | | CUGGCCGUCCUCGCCGUGGUGGUGAUUGGAGCUGUGGUCGCAGCUG |
| | | | UUAUGUGCAGAAGAAAGUCAUCCGGCGGAAAGGGAGGCUCCUACUC |
| | | | UCAGGCUGCUUCUGCUACAGUGCCUAGAGCUCUUAUGUGUUUAUCU |
| | | | CAGCUGGGCGGCGGAGGCAGCGACUACAAGGACGACGAUGACAAGU |
| | | | AA |
| 3068 | STARR | Signal | AUGAGAGUGACAGCCCCUAGAACCUUACUGCUUCUGCUUUGGGGAG |
| (SEQ | | peptide- | CUGUUGCUCUGACAGAGACAUGGGCUGGAUCUUACCACAGCCCCAG |
| ID | | AH1A5 OVA- | CUACGCCUACCACCAGUUCGAGAGGGGGGGAGGAGGCUCCGGGGGA |
| NO: 92) | | MITD-FLAG | GGAGGCUCCCUGAAGAUCAGCCAGGCCGUGCACGCCGCCCACGCCG |
| | | | AGAUCAACGAGGCCGGCCGGGAGGUGAUCGUGGGCAUUGUCGCUGG |
| | | | CCUGGCCGUCCUCGCCGUGGUGGUGAUUGGAGCUGUGGUCGCAGCU |
| | | | GUUAUGUGCAGAAGAAAGUCAUCCGGCGGAAAGGGAGGCUCCUACU |
| | | | CUCAGGCUGCUUCUGCUACAGUGCCUAGAGCUCUUAUGUGUUUAUC |
| | | | UCAGCUGGGCGGCGGAGGCAGCGACUACAAGGACGACGAUGACAAG |
| | | | UAA |

Transgene ORF amino acid sequence

| mARM | transgene | |
|---|---|---|
| # | description | Sequence |
| 2809, | Fluc | MEDAKNIKKGPAPFYPLEDGTAGEQLHKAMKRYALVPGTIAFTDAH |
| 2842, | | IEVDITYAEYFEMSVRLAEAMKRYGLNTNHRIVVCSENSLQFFMPV |
| 1782 | | LGALFIGVAVAPANDIYNERELLNSMGISQPTVVFVSKKGLQKILN |
| (SEQ | | VQKKLPIIQKIIIMDSKTDYQGFQSMYTFVTSHLPPGFNEYDFVPE |
| ID | | SFDRDKTIALIMNSSGSTGLPKGVALPHRTACVRFSHARDPIFGNQ |
| NO: 93) | | IIPDTAILSVVPFHHGFGMFTTLGYLICGFRVVLMYRFEEELFLRS |
| | | LQDYKIQSALLVPTLFSFFAKSTLIDKYDLSNLHEIASGGAPLSKE |
| | | VGEAVAKRFHLPGIRQGYGLTETTSAILITPEGDDKPGAVGKVVPF |
| | | FEAKVVDLDTGKTLGVNQRGELCVRGPMIMSGYVNNPEATNALIDK |
| | | DGWLHSGDIAYWDEDEHFFIVDRLKSLIKYKGYQVAPAELESILLQ |
| | | HPNIFDAGVAGLPDDDAGELPAAVVVLEHGKTMTEKEIVDYVASQV |
| | | TTAKKLRGGVVFVDEVPKGLTGKLDARKIREILIKAKKGGKIAV* |
| 2847 | KRAS | MKLVVVGAGGVGKSALTI* |
| (SEQ ID | epitope wt | |
| NO: 94) | | |
| 2862 | empty | MDPRRYAPMIRPAKLDVLPRN* |
| (SEQ ID | | |
| NO: 95) | | |
| 3060 | Signal | MRVTAPRTLLLLLWGAVALTETWAGSLSEVTGQGLCIGAVPKTHQV |
| (SEQ | peptide- | LCNTTQKTSDGSYYLAAPTGTTWACSTGLTPCISTTILNLTTDYCV |
| ID | gp70 with | LVELWPRVTYHSPSYAYHQFERRAKYKREPVSLTLALLLGGLTMGG |
| NO: 96) | AH1A5- | IAAGVGTGTTALVATQQFQQLQAAMHDDLKEVEKSITNLEKSLTSL |
| | MITD | SEVVLQNRRGLDLLFLKEGGLCAALKEECCLYADHTGLVIVGIVAG |
| | | LAVLAVVVIGAVVAAVMCRRKSSGGKGGSYSQAASATVPRALMCLS |
| | | QL* |
| 3061 | Signal | MRVTAPRTLLLLLWGAVALTETWAGSYHSPSYAYHQFERGGGGSGG |
| (SEQ | peptide- | GGSLKISQAVHAAHAEINEAGREVIVGIVAGLAVLAVVVIGAVVAA |
| ID | AH1A5 OVA- | VMCRRKSSGGKGGSYSQAASATVPRALMCLSQL* |
| NO: 97) | MITD | |
| 3076 | Signal | MRVTAPRTLLLLLWGAVALTETWAGSLSEVTGQGLCIGAVPKTHQV |
| (SEQ | peptide- | LCNTTQKTSDGSYYLAAPTGTTWACSTGLTPCISTTILNLTTDYCV |
| ID | gp70 with | LVELWPRVTYHSPSYAYHQFERRAKYKREPVSLTLALLLGGLTMGG |
| NO: 98) | AH1A5- | IAAGVGTGTTALVATQQFQQLQAAMHDDLKEVEKSITNLEKSLTSL |
| | MITD-FLAG | SEVVLQNRRGLDLLFLKEGGLCAALKEECCLYADHTGLVIVGIVAG |
| | | LAVLAVVVIGAVVAAVMCRRKSSGGKGGSYSQAASATVPRALMCLS |
| | | QLGGGGSDYKDDDDK* |
| 3068 | Signal | MRVTAPRTLLLLLWGAVALTETWAGSYHSPSYAYHQFERGGGGSGG |
| (SEQ | peptide- | GGSLKISQAVHAAHAEINEAGREVIVGIVAGLAVLAVVVIGAVVAA |
| NO: 99) | AH1A5 OVA- | VMCRRKSSGGKGGSYSQAASATVPRALMCLSQLGGGGSDYKDDDDK |
| | MITD-FLAG | * |

whole RNA sequence

| mARM | brief | | |
|---|---|---|---|
| # | name | | Sequence |
| 2809 | STARR^{TM} | 2809 | AUGGGCGGCGCAUGAGAGAAGCCCAGACCAAUUACCUACCCAAAAU |
| (SEQ | Fluc | | GGAGAAAGUUCACGUUGACAUCGAGGAAGACAGCCCAUUCCUCAGA |
| ID | | | GCUUUGCAGCGGAGCUUCCCGCAGUUUGAGGUAGAAGCCAAGCAGG |
| NO: 100) | | | UCACUGAUAAUGACCAUGCUAAUGCCAGAGCGUUUUCGCAUCUGGC |
| | | | UUCAAAACUGAUCGAAACGGAGGUGGACCCAUCCGACACGAUCCUU |
| | | | GACAUUGGAAGUGCGCCCGCCCGCAGAAUGUAUUCUAAGCACAAGU |
| | | | AUCAUUGUAUCUGUCCGAUGAGAUGUGCGGAAGAUCCGGACAGAUU |
| | | | GUAUAAGUAUGCAACUAAGCUGAAGAAAAACUGUAAGGAAAUAACU |
| | | | GAUAAGGAAUUGGACAAGAAAAUGAAGGAGCUGGCCGCCGUCAUGA |
| | | | GCGACCCUGACCUGGAAACUGAGACUAUGUGCCUCCACGACGACGA |
| | | | GUCGUGUCGCUACGAAGGGCAAGUCGCUGUUUACCAGGAUGUAUAC |
| | | | GCCGUCGACGGCCCCACCAGCCUGUACCACCAGGCCAACAAGGGCG |
| | | | UGAGGGUGGCCUACUGGAUCGGCUUCGACACCACACCCUUCAUGUU |
| | | | CAAGAACCUGGCCGGCGCCUACCCCAGCUACAGCACCAACUGGGCC |
| | | | GACGAGACCGUGCUGACCGCCAGGAACAUCGGCCUGUGCAGCAGCG |
| | | | ACGUGAUGGAGAGGAGCCGGAGAGGCAUGAGCAUCCUGAGGAAGAA |
| | | | AUACCUGAAGCCCAGCAACAACGUGCUGUUCAGCGUGGGCAGCACC |
| | | | AUCUACCACGAGAAGAGGGACCUGCUCAGGAGCUGGCACCUGCCCA |
| | | | GCGUGUUCCACCUGAGGGGCAAGCAGAACUACACCUGCAGGUGCGA |
| | | | GACCAUCGUGAGCUGCGACGGCUACGUGGUGAAGAGGAUCGCCAUC |
| | | | AGCCCCGGCCUGUACGGCAAGCCCAGCGGCUACGCCGCUACAAUGC |
| | | | ACAGGGAGGGCUUCCUGUGCUGCAAGGUGACCGACACCCUGAACGG |
| | | | CGAGAGGGUGAGCUUCCCCGUGUGCACCUACGUGCCCGCCACCCUG |
| | | | UGCGACCAGAUGACCGGCAUCCUGGCCACCGACGUGAGCGCCGACG |
| | | | ACGCCCAGAAGCUGCUCGUGGGCCUGAACCAGAGGAUCGUGGUCAA |
| | | | CGGCAGGACCCAGAGGAACACCAACACAAUGAAGAACUACCUGCUG |
| | | | CCCGUGGUGGCCCAGGCUUUCGCCAGGUGGGCCAAGGAGUACAAGG |
| | | | AGGACCAGGAAGACGAGAGGCCCCUGGGCCUGAGGGACAGGCAGCU |
| | | | GGUGAUGGGCUGCUGCUGGGCCUUCAGGCGGCACAAGAUCACCAGC |
| | | | AUCUACAAGAGGCCCGACACCCAGACCAUCAUCAAGGUGAACAGCG |
| | | | ACUUCCACAGCUUCGUGCUGCCCAGGAUCGGCAGCAACACCCUGGA |
| | | | GAUCGGCCUGAGGACCCGGAUCAGGAAGAUGCUGGAGGAACACAAG |
| | | | GAGCCCAGCCCACUGAUCACCGCCGAGGACGUGCAGGAGGCCAAGU |
| | | | GCGCUGCCGACGAGGCCAAGGAGGUGAGGGAGGCCGAGGAACUGAG |
| | | | GGCCGCCCUGCCACCCCUGGCUGCCGACGUGGAGGAACCCACCCUG |
| | | | GAAGCCGACGUGGACCUGAUGCUGCAGGAGGCCGGCGCCGGAAGCG |
| | | | UGGAGACACCCAGGGGCCUGAUCAAGGUGACCAGCUACGACGGCGA |
| | | | GGACAAGAUCGGCAGCUACGCCGUGCUGAGCCCACAGGCCGUGCUG |
| | | | AAGUCCGAGAAGCUGAGCUGCAUCCACCCACUGGCCGAGCAGGUGA |
| | | | UCGUGAUCACCCACAGCGGCAGGAAGGGCAGGUACGCCGUGGAGCC |
| | | | CUACCACGGCAAGGUGGUCGUGCCCGAGGGCCACGCCAUCCCCGUG |
| | | | CAGGACUUCCAGGCCCUGAGCGAGAGCGCCACCAUCGUGUACAACG |
| | | | AGAGGGAGUUCGUGAACAGGUACCUGCACCAUAUCGCCACCCACGG |
| | | | CGGAGCCCUGAACACCGACGAGGAAUACUACAAGACCGUGAAGCCC |
| | | | AGCGAGCACGACGGCGAGUACCUGUACGACAUCGACAGGAAGCAGU |
| | | | GCGUGAAGAAAGAGCUGGUGACCGGCCUGGGACUGACCGGCGAGCU |
| | | | GGUGGACCCACCCUUCCACGAGUUCGCCUACGAGAGCCUGAGGACC |
| | | | AGACCCGCCGCUCCCUACCAGGUGCCCACCAUCGGCGUGUACGGCG |
| | | | UGCCCGGCAGCGGAAAGAGCGGCAUCAUCAAGAGCGCCGUGACCAA |
| | | | GAAAGACCUGGUGGUCAGCGCCAAGAAAGAGAACUGCGCCGAGAUC |
| | | | AUCAGGGACGUGAAGAAGAUGAAAGGCCUGGACGUGAACGCGCGCA |
| | | | CCGUGGACAGCGUGCUGCUGAACGGCUGCAAGCACCCCGUGGAGAC |
| | | | CCUGUACAUCGACGAGGCCUUCGCUUGCCACGCCGGCACCCUGAGG |
| | | | GCCCUGAUCGCCAUCAUCAGGCCCAAGAAAGCCGUGCUGUGCGGCG |
| | | | ACCCCAAGCAGUGCGGCUUCUUCAACAUGAUGUGCCUGAAGGUGCA |
| | | | CUUCAACCACGAGAUCUGCACCCAGGUGUUCCACAAGAGCAUCAGC |
| | | | AGGCGGUGCACCAAGAGCGUGACCAGCGUCGUGAGCACCCUGUUCU |
| | | | ACGACAAGAAAAUGAGGACCACCAACCCCAAGGAGACCAAAAUCGU |
| | | | GAUCGACACCACAGGCAGCACCAAGCCCAAGCAGGACGACCUGAUC |
| | | | CUGACCUGCUUCAGGGGCUGGGUGAAGCAGCUGCAGAUCGACUACA |
| | | | AGGGCAACGAGAUCAUGACCGCCGCUGCCAGCCAGGGCCUGACCAG |
| | | | GAAGGGCGUGUACGCCGUGAGGUACAAGGUGAACGAGAACCCACUG |
| | | | UACGCUCCCACCAGCGAGCACGUGAACGUGCUGCUGACCAGGACCG |
| | | | AGGACAGGAUCGUGUGGAAGACCCUGGCCGGCGACCCCUGGAUCAA |
| | | | GACCCUGACCGCCAAGUACCCCGGCAACUUCACCGCCACCAUCGAA |
| | | | GAGUGGCAGGCCGAGCACGACGCCAUCAUGAGGCACAUCCUGGAGA |
| | | | GGCCCGACCCCACCGACGUGUUCCAGAACAAGGCCAACGUGUGCUG |
| | | | GGCCAAGGCCCUGGUGCCCGUGCUGAAGACCGCCGGCAUCGACAUG |
| | | | ACCACAGAGCAGUGGAACACCGUGGACUACUUCGAGACCGACAAGG |
| | | | CCCACAGCGCCGAGAUCGUGCUGAACCAGCUGUGCGUGAGGUUCUU |
| | | | CGGCCUGGACCUGGACAGCGGCCUGUUCAGCGCCCCCACCGUGCCA |
| | | | CUGAGCAUCAGGAACAACCACUGGGACAACAGCCCCAGCCCAAACA |
| | | | UGUACGGCCUGAACAAGGAGGUGGUCAGGCAGCUGAGCAGGCGGUA |
| | | | CCCACAGCUGCCCAGGGCCGUGGCCACCGGCAGGGUGUACGACAUG |
| | | | AACACCGGCACCCUGAGGAACUACGACCCCAGGAUCAACCUGGUGC |
| | | | CCGUGAACAGGCGGCUGCCCCACGCCCUGGUGCUGCACCACAACGA |
| | | | GCACCCACAGAGCGACUUCAGCUCCUUCGUGAGCAAGCUGAAAGGC |
| | | | AGGACCGUGCUGGUCGUGGGCGAGAAGCUGAGCGUGCCCGGCAAGA |
| | | | UGGUGGACUGGCUGAGCGACAGGCCCGAGGCCACCUUCCGGGCCAG |
| | | | GCUGGACCUCGGCAUCCCCGGCGACGUGCCCAAGUACGACAUCAUC |
| | | | UUCGUGAACGUCAGGACCCCAUACAAGUACCACCAUUACCAGCAGU |
| | | | GCGAGGACCACGCCAUCAAGCUGAGCAUGCUGACCAAGAAGGCCUG |
| | | | CCUGCACCUGAACCCCGGAGGCACCUGCGUGAGCAUCGGCUACGGC |
| | | | UACGCCGACAGGGCCAGCGAGAGCAUCAUUGGCGCCAUCGCCAGGC |
| | | | UGUUCAAGUUCAGCAGGGUGUGCAAACCCAAGAGCAGCCUGGAGGA |
| | | | AACCGAGGUGCUGUUCGUGUUCAUCGGCUACGACCGGAAGGCCAGG |
| | | | ACCCACAACCCCUACAAGCUGAGCAGCACCCUGACAAACAUCUACA |
| | | | CCGGCAGCAGGCUGCACGAGGCCGGCUGCGCCCCCAGCUACCACGU |
| | | | GGUCAGGGGCGAUAUCGCCACCGCCACCGAGGGCGUGAUCAUCAAC |
| | | | GCUGCCAACAGCAAGGGCCAGCCCGGAGGCGGAGUGUGCGGCGCCC |
| | | | UGUACAAGAAGUUCCCCGAGAGCUUCGACCUGCAGCCCAUCGAGGU |
| | | | GGGCAAGGCCAGGCUGGUGAAGGGCGCCGCUAAGCACAUCAUCCAC |
| | | | GCCGUGGGCCCCAACUUCAACAAGGUGAGCGAGGUGGAAGGCGACA |
| | | | AGCAGCUGGCCGAAGCCUACGAGAGCAUCGCCAAGAUCGUGAACGA |
| | | | CAAUAACUACAAGAGCGUGGCCAUCCCACUGCUCAGCACCGGCAUC |
| | | | UUCAGCGGCAACAAGGACAGGCUGACCCAGAGCCUGAACCACCUGC |
| | | | UCACCGCCCUGGACACCACCGAUGCCGACGUGGCCAUCUACUGCAG |
| | | | GGACAAGAAGUGGGAGAUGACCCUGAAGGAGGCCGUGGCCAGGCGG |
| | | | GAGGCCGUGGAAGAGAUCUGCAUCAGCGACGACUCCAGCGUGACCG |
| | | | AGCCCGACGCCGAGCUGGUGAGGGUGCACCCCAAGAGCUCCCUGGC |
| | | | CGGCAGGAAGGGCUACAGCACCAGCGACGGCAAGACCUUCAGCUAC |
| | | | CUGGAGGGCACCAAGUUCCACCAGGCCGCUAAGGACAUCGCCGAGA |
| | | | UCAACGCUAUGUGGCCCGUGGCCACCGAGGCCAACGAGCAGGUGUG |
| | | | CAUGUACAUCCUGGGCGAGAGCAUGUCCAGCAUCAGGAGCAAGUGC |
| | | | CCCGUGGAGGAAAGCGAGGCCAGCACACCACCCAGCACCCUGCCCU |
| | | | GCCUGUGCAUCCACGCUAUGACACCCGAGAGGGUGCAGCGGCUGAA |
| | | | GGCCAGCAGGCCCGAGCAGAUCACCGUGUGCAGCUCCUUCCCACUG |
| | | | CCCAAGUACAGGAUCACCGGCGUGCAGAAGAUCCAGUGCAGCCAGC |
| | | | CCAUCCUGUUCAGCCCAAAGGUGCCCGCCUACAUCCACCCCAGGAA |
| | | | GUACCUGGUGGAGACCCCACCCGUGGACGAGACACCCGAGCCAAGC |
| | | | GCCGAGAACCAGAGCACCGAGGGCACACCCGAGCAGCCACCCCUGA |
| | | | UCACCGAGGACGAGACAAGGACCCGGACCCCAGAGCCCAUCAUUAU |
| | | | CGAGGAAGAGGAAGAGGACAGCAUCAGCCUGCUGAGCGACGGCCCC |
| | | | ACCCACCAGGUGCUGCAGGUGGAGGCCGACAUCCACGGCCCACCCA |
| | | | GCGUGUCCAGCUCCAGCUGGAGCAUCCCACACGCCAGCGACUUCGA |
| | | | CGUGGACAGCCUGAGCAUCCUGGACACCCUGGAGGGCGCCAGCGUG |
| | | | ACCUCCGGCGCCACCAGCGCCGAGACCAACAGCUACUUCGCCAAGA |
| | | | GCAUGGAGUUCCUGGCCAGGCCCGUGCCAGCUCCCAGGACCGUGUU |
| | | | CAGGAACCCACCCCACCCAGCUCCCAGGACCAGGACCCCAAGCCUG |
| | | | GCUCCCAGCAGGGCCUGCAGCAGGACCAGCCUGGUGAGCACCCCAC |
| | | | CCGGCGUGAACAGGGUGAUCACCAGGGAGGAACUGGAGGCCCUGAC |
| | | | ACCCAGCAGGACCCCCAGCAGGUCCGUGAGCAGGACUAGUCUGGUG |
| | | | UCCAACCCACCCGGCGUGAACAGGGUGAUCACCAGGGAGGAAUUCG |
| | | | AGGCCUUCGUGGCCCAGCAACAGAGACGGUUCGACGCCGGCGCCUA |
| | | | CAUCUUCAGCAGCGACACCGGCCAGGGACACCUGCAGCAAAAGAGC |
| | | | GUGAGGCAGACCGUGCUGAGCGAGGUGGUGCUGGAGAGGACCGAGC |
| | | | UGGAAAUCAGCUACGCCCCCAGGCUGGACCAGGAGAAGGAGGAACU |
| | | | GCUCAGGAAGAAACUGCAGCUGAACCCCACCCCAGCCAACAGGAGC |
| | | | AGGUACCAGAGCAGGAAGGUGGAGAACAUGAAGGCCAUCACCGCCA |
| | | | GGCGGAUCCUGCAGGGCCUGGGACACUACCUGAAGGCCGAGGGCAA |
| | | | GGUGGAGUGCUACAGGACCCUGCACCCCGUGCCACUGUACAGCUCC |
| | | | AGCGUGAACAGGGCCUUCUCCAGCCCCAAGGUGGCCGUGGAGGCCU |
| | | | GCAACGCUAUGCUGAAGGAGAACUUCCCCACCGUGGCCAGCUACUG |
| | | | CAUCAUCCCCGAGUACGACGCCUACCUGGACAUGGUGGACGGCGCC |
| | | | AGCUGCUGCCUGGACACCGCCAGCUUCUGCCCCGCCAAGCUGAGGA |
| | | | GCUUCCCCAAGAAACACAGCUACCUGGAGCCCACCAUCAGGAGCGC |
| | | | CGUGCCCAGCGCCAUCCAGAACACCCUGCAGAACGUGCUGGCCGCU |
| | | | GCCACCAAGAGGAACUGCAACGUGACCCAGAUGAGGGAGCUGCCCG |
| | | | UGCUGGACAGCGCUGCCUUCAACGUGGAGUGCUUCAAGAAAUACGC |
| | | | CUGCAACAACGAGUACUGGGAGACCUUCAAGGAGAACCCCAUCAGG |
| | | | CUGACCGAAGAGAACGUGGUGAACUACAUCACCAAGCUGAAGGGCC |
| | | | CCAAGGCCGCUGCCCUGUUCGCUAAGACCCACAACCUGAACAUGCU |
| | | | GCAGGACAUCCCAAUGGACAGGUUCGUGAUGGACCUGAAGAGGGAC |
| | | | GUGAAGGUGACACCCGGCACCAAGCACACCGAGGAGAGGCCCAAGG |
| | | | UGCAGGUGAUCCAGGCCGCUGACCCACUGGCCACCGCCUACCUGUG |
| | | | CGGCAUCCACAGGGAGCUGGUGAGGCGGCUGAACGCCGUGCUGCUG |
| | | | CCCAACAUCCACACCCUGUUCGACAUGAGCGCCGAGGACUUCGACG |
| | | | CCAUCAUCGCCGAGCACUUCCAGCCCGGCGACUGCGUGCUGGAGAC |
| | | | CGACAUCGCCAGCUUCGACAAGAGCGAGGAUGACGCUAUGGCCCUG |
| | | | ACCGCUCUGAUGAUCCUGGAGGACCUGGGCGUGGACGCCGAGCUGC |
| | | | UCACCCUGAUCGAGGCUGCCUUCGGCGAGAUCAGCUCCAUCCACCU |
| | | | GCCCACCAAGACCAAGUUCAAGUUCGGCGCUAUGAUGAAAAGCGGA |
| | | | AUGUUCCUGACCCUGUUCGUGAACACCGUGAUCAACAUUGUGAUCG |
| | | | CCAGCAGGGUGCUGCGGGAGAGGCUGACCGGCAGCCCCUGCGCUGC |
| | | | CUUCAUCGGCGACGACAACAUCGUGAAGGGCGUGAAAAGCGACAAG |
| | | | CUGAUGGCCGACAGGUGCGCCACCUGGCUGAACAUGGAGGUGAAGA |
| | | | UCAUCGACGCCGUGGUGGGCGAGAAGGCCCCCUACUUCUGCGGCGG |
| | | | AUUCAUCCUGUGCGACAGCGUGACCGGCACCGCCUGCAGGGUGGCC |
| | | | GACCCCCUGAAGAGGCUGUUCAAGCUGGGCAAGCCACUGGCCGCUG |
| | | | ACGAUGAGCACGACGAUGACAGGCGGAGGGCCCUGCACGAGGAAAG |
| | | | CACCAGGUGGAACAGGGUGGGCAUCCUGAGCGAGCUGUGCAAGGCC |
| | | | GUGGAGAGCAGGUACGAGACCGUGGGCACCAGCAUCAUCGUGAUGG |
| | | | CUAUGACCACACUGGCCAGCUCCGUCAAGAGCUUCUCCUACCUGAG |
| | | | GGGGGCCCCUAUAACUCUCUACGGCUAACCUGAAUGGACUACGACA |
| | | | UAGUCUAGUCCGCCAAGGCCGCCACCAUGGAAGAUGCCAAAAACAU |
| | | | UAAGAAGGGCCCAGCGCCAUUCUACCCACUCGAAGACGGGACCGCC |
| | | | GGCGAGCAGCUGCACAAAGCCAUGAAGCGCUACGCCCUGGUGCCCG |
| | | | GCACCAUCGCCUUUACCGACGCACAUAUCGAGGUGGACAUUACCUA |
| | | | CGCCGAGUACUUCGAGAUGAGCGUUCGGCUGGCAGAAGCUAUGAAG |
| | | | CGCUAUGGGCUGAAUACAAACCAUCGGAUCGUGGUGUGCAGCGAGA |
| | | | AUAGCUUGCAGUUCUUCAUGCCCGUGUUGGGUGCCCUGUUCAUCGG |
| | | | UGUGGCUGUGGCCCCAGCUAACGACAUCUACAACGAGCGCGAGCUG |
| | | | CUGAACAGCAUGGGCAUCAGCCAGCCCACCGUCGUAUUCGUGAGCA |
| | | | AGAAAGGGCUGCAAAAGAUCCUCAACGUGCAAAAGAAGCUACCGAU |
| | | | CAUACAAAAGAUCAUCAUCAUGGAUAGCAAGACCGACUACCAGGGC |
| | | | UUCCAAAGCAUGUACACCUUCGUGACUUCCCAUUUGCCACCCGGCU |
| | | | UCAACGAGUACGACUUCGUGCCCGAGAGCUUCGACCGGGACAAAAC |
| | | | CAUCGCCCUGAUCAUGAACAGUAGUGGCAGUACCGGAUUGCCCAAG |
| | | | GGCGUAGCCCUACCGCACCGCACCGCUUGUGUCCGAUUCAGUCAUG |
| | | | CCCGCGACCCCAUCUUCGGCAACCAGAUCAUCCCCGACACCGCUAU |
| | | | CCUCAGCGUGGUGCCAUUUCACCACGGCUUCGGCAUGUUCACCACG |
| | | | CUGGGCUACUUGAUCUGCGGCUUUCGGGUCGUGCUCAUGUACCGCU |
| | | | UCGAGGAGGAGCUAUUCUUGCGCAGCUUGCAAGACUAUAAGAUUCA |
| | | | AUCUGCCCUGCUGGUGCCCACACUAUUUAGCUUCUUCGCUAAGAGC |
| | | | ACUCUCAUCGACAAGUACGACCUAAGCAACUUGCACGAGAUCGCCA |
| | | | GCGGCGGGGCGCCGCUCAGCAAGGAGGUAGGUGAGGCCGUGGCCAA |
| | | | ACGCUUCCACCUACCAGGCAUCCGACAGGGCUACGGCCUGACAGAA |
| | | | ACAACCAGCGCCAUUCUGAUCACCCCCGAAGGGGACGACAAGCCUG |
| | | | GCGCAGUAGGCAAGGUGGUGCCCUUCUUCGAGGCUAAGGUGGUGGA |
| | | | CUUGGACACCGGUAAGACACUGGGUGUGAACCAGCGCGGCGAGCUG |
| | | | UGCGUCCGUGGCCCCAUGAUCAUGAGCGGCUACGUUAACAACCCCG |
| | | | AGGCUACAAACGCUCUCAUCGACAAGGACGGCUGGCUGCACAGCGG |
| | | | CGACAUCGCCUACUGGGACGAGGACGAGCACUUCUUCAUCGUGGAC |
| | | | CGGCUGAAGUCCCUGAUCAAAUACAAGGGCUACCAGGUAGCCCCAG |
| | | | CCGAACUGGAGAGCAUCCUGCUGCAACACCCCAACAUCUUCGACGC |
| | | | CGGGGUCGCCGGCCUGCCCGACGACGAUGCCGGCGAGCUGCCCGCC |
| | | | GCAGUCGUCGUGCUGGAACACGGUAAAACCAUGACCGAGAAGGAGA |
| | | | UCGUGGACUAUGUGGCCAGCCAGGUUACAACCGCCAAGAAGCUGCG |
| | | | CGGUGGUGUUGUGUUCGUGGACGAGGUGCCUAAAGGACUGACCGGC |
| | | | AAGUUGGACGCCCGCAAGAUCCGCGAGAUUCUCAUUAAGGCCAAGA |
| | | | AGGGCGGCAAGAUCGCCGUGUAACUCGAGUAUGUUACGUGCAAAGG |
| | | | UGAUUGUCACCCCCCGAAAGACCAUAUUGUGACACACCCUCAGUAU |
| | | | CACGCCCAAACAUUUACAGCCGCGGUGUCAAAAACCGCGUGGACGU |
| | | | GGUUAACAUCCCUGCUGGGAGGAUCAGCCGUAAUUAUUAUAAUUGG |
| | | | CUUGGUGCUGGCUACUAUUGUGGCCAUGUACGUGCUGACCAACCAG |
| | | | AAACAUAAUUGAAUACAGCAGCAAUUGGCAAGCUGCUUACAUAGAA |
| | | | CUCGCGGCGAUUGGCAUGCCGCCUUAAAAUUUUUAUUUUAUUUUUU |
| | | | CUUUUCUUUUCCGAAUCGGAUUUUGUUUUUAAUAUUUCAAAAAAAA |
| | | | AAAAAAAAAAAAAAAAAUCUAGAAAAAAAAAAAAAAAAAAAAAAAA |
| | | | AAAAAAAAAAAAAAAAAAAAAAAAAAAAAAAAAAAAAAAAAAAAAA |
| | | | AAAAAAAAAAAAAAAAAAAAAAAAAAAAAA |
| 2842 | SINV | 2842 | AUUGACGGCGUAGUACACACUAUUGAAUCAAACAGCCGACCAAUUG |
| (SEQ | Fluc | | CACUACCAUCACAAUGGAGAAGCCAGUAGUAAACGUAGACGUAGAC |
| ID | | | CCCCAGAGUCCGUUUGUCGUGCAACUGCAAAAAAGCUUCCCGCAAU |
| NO: 101) | | | UUGAGGUAGUAGCACAGCAGGUCACUCCAAAUGACCAUGCUAAUGC |
| | | | CAGAGCAUUUUCGCAUCUGGCCAGUAAACUAAUCGAGCUGGAGGUU |
| | | | CCUACCACAGCGACGAUCUUGGACAUAGGCAGCGCACCGGCUCGUA |
| | | | GAAUGUUUUCCGAGCACCAGUAUCAUUGUGUCUGCCCCAUGCGUAG |
| | | | UCCAGAAGACCCGGACCGCAUGAUGAAAUAUGCCAGUAAACUGGCG |
| | | | GAAAAAGCGUGCAAGAUUACAAACAAGAACUUGCAUGAGAAGAUUA |
| | | | AGGAUCUCCGGACCGUACUUGAUACGCCGGAUGCUGAAACACCAUC |
| | | | GCUCUGCUUUCACAACGAUGUUACCUGCAACAUGCGUGCCGAAUAU |
| | | | UCCGUCAUGCAGGACGUGUAUAUCAACGCUCCCGGAACUAUCUAUC |
| | | | AUCAGGCUAUGAAAGGCGUGCGGACCCUGUACUGGAUUGGCUUCGA |
| | | | CACCACCCAGUUCAUGUUCUCGGCUAUGGCAGGUUCGUACCCUGCG |
| | | | UACAACACCAACUGGGCCGACGAGAAAGUCCUUGAAGCGCGUAACA |
| | | | UCGGACUUUGCAGCACAAAGCUGAGUGAAGGUAGGACAGGAAAAUU |
| | | | GUCGAUAAUGAGGAAGAAGGAGUUGAAGCCCGGGUCGCGGGUUUAU |
| | | | UUCUCCGUAGGAUCGACACUUUAUCCAGAACACAGAGCCAGCUUGC |
| | | | AGAGCUGGCAUCUUCCAUCGGUGUUCCACUUGAAUGGAAAGCAGUC |
| | | | GUACACUUGCCGCUGUGAUACAGUGGUGAGUUGCGAAGGCUACGUA |
| | | | GUGAAGAAAAUCACCAUCAGUCCCGGGAUCACGGGAGAAACCGUGG |
| | | | GAUACGCGGUUACACACAAUAGCGAGGGCUUCUUGCUAUGCAAAGU |
| | | | UACUGACACAGUAAAAGGAGAACGGGUAUCGUUCCCUGUGUGCACG |
| | | | UACAUCCCGGCCACCAUAUGCGAUCAGAUGACUGGUAUAAUGGCCA |
| | | | CGGAUAUAUCACCUGACGAUGCACAAAAACUUCUGGUUGGGCUCAA |
| | | | CCAGCGAAUUGUCAUUAACGGUAGGACUAACAGGAACACCAACACC |
| | | | AUGCAAAAUUACCUUCUGCCGAUCAUAGCACAAGGGUUCAGCAAAU |
| | | | GGGCUAAGGAGCGCAAGGAUGAUCUUGAUAACGAGAAAAUGCUGGG |
| | | | UACUAGAGAACGCAAGCUUACGUAUGGCUGCUUGUGGGCGUUUCGC |
| | | | ACUAAGAAAGUACAUUCGUUUUAUCGCCCACCUGGAACGCAGACCU |
| | | | GCGUAAAAGUCCCAGCCUCUUUUAGCGCUUUUCCCAUGUCGUCCGU |
| | | | AUGGACGACCUCUUUGCCCAUGUCGCUGAGGCAGAAAUUGAAACUG |
| | | | GCAUUGCAACCAAAGAAGGAGGAAAAACUGCUGCAGGUCUCGGAGG |
| | | | AAUUAGUCAUGGAGGCCAAGGCUGCUUUUGAGGAUGCUCAGGAGGA |
| | | | AGCCAGAGCGGAGAAGCUCCGAGAAGCACUUCCACCAUUAGUGGCA |
| | | | GACAAAGGCAUCGAGGCAGCCGCAGAAGUUGUCUGCGAAGUGGAGG |
| | | | GGCUCCAGGCGGACAUCGGAGCAGCAUUAGUUGAAACCCCGCGCGG |
| | | | UCACGUAAGGAUAAUACCUCAAGCAAAUGACCGUAUGAUCGGACAG |
| | | | UAUAUCGUUGUCUCGCCAAACUCUGUGCUGAAGAAUGCCAAACUCG |
| | | | CACCAGCGCACCCGCUAGCAGAUCAGGUUAAGAUCAUAACACACUC |
| | | | CGGAAGAUCAGGAAGGUACGCGGUCGAACCAUACGACGCUAAAGUA |
| | | | CUGAUGCCAGCAGGAGGUGCCGUACCAUGGCCAGAAUUCCUAGCAC |
| | | | UGAGUGAGAGCGCCACGUUAGUGUACAACGAAAGAGAGUUUGUGAA |
| | | | CCGCAAACUAUACCACAUUGCCAUGCAUGGCCCCGCCAAGAAUACA |
| | | | GAAGAGGAGCAGUACAAGGUUACAAAGGCAGAGCUUGCAGAAACAG |
| | | | AGUACGUGUUUGACGUGGACAAGAAGCGUUGCGUUAAGAAGGAAGA |
| | | | AGCCUCAGGUCUGGUCCUCUCGGGAGAACUGACCAACCCUCCCUAU |
| | | | CAUGAGCUAGCUCUGGAGGGACUGAAGACCCGACCUGCGGUCCCGU |
| | | | ACAAGGUCGAAACAAUAGGAGUGAUAGGCACACCGGGGUCGGGCAA |
| | | | GUCAGCUAUUAUCAAGUCAACUGUCACGGCACGAGAUCUUGUUACC |
| | | | AGCGGAAAGAAAGAAAAUUGUCGCGAAAUUGAGGCCGACGUGCUAA |
| | | | GACUGAGGGGUAUGCAGAUUACGUCGAAGACAGUAGAUUCGGUUAU |
| | | | GCUCAACGGAUGCCACAAAGCCGUAGAAGUGCUGUACGUUGACGAA |
| | | | GCGUUCGCGUGCCACGCAGGAGCACUACUUGCCUUGAUUGCUAUCG |
| | | | UCAGGCCCCGCAAGAAGGUAGUACUAUGCGGAGACCCCAUGCAAUG |
| | | | CGGAUUCUUCAACAUGAUGCAACUAAAGGUACAUUUCAAUCACCCU |
| | | | GAAAAAGACAUAUGCACCAAGACAUUCUACAAGUAUAUCUCCCGGC |
| | | | GUUGCACACAGCCAGUUACAGCUAUUGUAUCGACACUGCAUUACGA |
| | | | UGGAAAGAUGAAAACCACGAACCCGUGCAAGAAGAACAUUGAAAUC |
| | | | GAUAUUACAGGGGCCACAAAGCCGAAGCCAGGGGAUAUCAUCCUGA |
| | | | CAUGUUUCCGCGGGUGGGUUAAGCAAUUGCAAAUCGACUAUCCCGG |
| | | | ACAUGAAGUAAUGACAGCCGCGGCCUCACAAGGGCUAACCAGAAAA |
| | | | GGAGUGUAUGCCGUCCGGCAAAAAGUCAAUGAAAACCCACUGUACG |
| | | | CGAUCACAUCAGAGCAUGUGAACGUGUUGCUCACCCGCACUGAGGA |
| | | | CAGGCUAGUGUGGAAAACCUUGCAGGGCGACCCAUGGAUUAAGCAG |
| | | | CUCACUAACAUACCUAAAGGAAACUUUCAGGCUACUAUAGAGGACU |
| | | | GGGAAGCUGAACACAAGGGAAUAAUUGCUGCAAUAAACAGCCCCAC |
| | | | UCCCCGUGCCAAUCCGUUCAGCUGCAAGACCAACGUUUGCUGGGCG |
| | | | AAAGCAUUGGAACCGAUACUAGCCACGGCCGGUAUCGUACUUACCG |
| | | | GUUGCCAGUGGAGCGAACUGUUCCCACAGUUUGCGGAUGACAAACC |
| | | | ACAUUCGGCCAUUUACGCCUUAGACGUAAUUUGCAUUAAGUUUUUC |
| | | | GGCAUGGACUUGACAAGCGGACUGUUUUCUAAACAGAGCAUCCCAC |
| | | | UAACGUACCAUCCCGCCGAUUCAGCGAGGCCGGUAGCUCAUUGGGA |
| | | | CAACAGCCCAGGAACCCGCAAGUAUGGGUACGAUCACGCCAUUGCC |
| | | | GCCGAACUCUCCCGUAGAUUUCCGGUGUUCCAGCUAGCUGGGAAGG |
| | | | GCACACAACUUGAUUUGCAGACGGGGAGAACCAGAGUUAUCUCUGC |
| | | | ACAGCAUAACCUGGUCCCGGUGAACCGCAAUCUUCCUCACGCCUUA |
| | | | GUCCCCGAGUACAAGGAGAAGCAACCCGGCCCGGUCGAAAAAUUCU |
| | | | UGAACCAGUUCAAACACCACUCAGUACUUGUGGUAUCAGAGGAAAA |
| | | | AAUUGAAGCUCCCCGUAAGAGAAUCGAAUGGAUCGCCCCGAUUGGC |
| | | | AUAGCCGGUGCAGAUAAGAACUACAACCUGGCUUUCGGGUUUCCGC |
| | | | CGCAGGCACGGUACGACCUGGUGUUCAUCAACAUUGGAACUAAAUA |
| | | | CAGAAACCACCACUUUCAGCAGUGCGAAGACCAUGCGGCGACCUUA |
| | | | AAAACCCUUUCGCGUUCGGCCCUGAAUUGCCUUAACCCAGGAGGCA |
| | | | CCCUCGUGGUGAAGUCCUAUGGCUACGCCGACCGCAACAGUGAGGA |
| | | | CGUAGUCACCGCUCUUGCCAGAAAGUUUGUCAGGGUGUCUGCAGCG |
| | | | AGACCAGAUUGUGUCUCAAGCAAUACAGAAAUGUACCUGAUUUUCC |
| | | | GACAACUAGACAACAGCCGUACACGGCAAUUCACCCCGCACCAUCU |
| | | | GAAUUGCGUGAUUUCGUCCGUGUAUGAGGGUACAAGAGAUGGAGUU |
| | | | GGAGCCGCGCCGUCAUACCGCACCAAAAGGGAGAAUAUUGCUGACU |
| | | | GUCAAGAGGAAGCAGUUGUCAACGCAGCCAAUCCGCUGGGUAGACC |
| | | | AGGCGAAGGAGUCUGCCGUGCCAUCUAUAAACGUUGGCCGACCAGU |
| | | | UUUACCGAUUCAGCCACGGAGACAGGCACCGCAAGAAUGACUGUGU |
| | | | GCCUAGGAAAGAAAGUGAUCCACGCGGUCGGCCCUGAUUUCCGGAA |
| | | | GCACCCAGAAGCAGAAGCCUUGAAAUUGCUACAAAACGCCUACCAU |
| | | | GCAGUGGCAGACUUAGUAAAUGAACAUAACAUCAAGUCUGUCGCCA |
| | | | UUCCACUGCUAUCUACAGGCAUUUACGCAGCCGGAAAAGACCGCCU |
| | | | UGAAGUAUCACUUAACUGCUUGACAACCGCGCUAGACAGAACUGAC |
| | | | GCGGACGUAACCAUCUAUUGCCUGGAUAAGAAGUGGAAGGAAAGAA |
| | | | UCGACGCGGCACUCCAACUUAAGGAGUCUGUAACAGAGCUGAAGGA |
| | | | UGAAGAUAUGGAGAUCGACGAUGAGUUAGUAUGGAUCCAUCCAGAC |
| | | | AGUUGCUUGAAGGGAAGAAAGGGAUUCAGUACUACAAAAGGAAAAU |
| | | | UGUAUUCGUACUUCGAAGGCACCAAAUUCCAUCAAGCAGCAAAAGA |
| | | | CAUGGCGGAGAUAAAGGUCCUGUUCCCUAAUGACCAGGAAAGUAAU |
| | | | GAACAACUGUGUGCCUACAUAUUGGGUGAGACCAUGGAAGCAAUCC |
| | | | GCGAAAAGUGCCCGGUCGACCAUAACCCGUCGUCUAGCCCGCCCAA |
| | | | AACGUUGCCGUGCCUUUGCAUGUAUGCCAUGACGCCAGAAAGGGUC |
| | | | CACAGACUUAGAAGCAAUAACGUCAAAGAAGUUACAGUAUGCUCCU |
| | | | CCACCCCCCUUCCUAAGCACAAAAUUAAGAAUGUUCAGAAGGUUCA |
| | | | GUGCACGAAAGUAGUCCUGUUUAAUCCGCACACUCCCGCAUUCGUU |
| | | | CCCGCCCGUAAGUACAUAGAAGUGCCAGAACAGCCUACCGCUCCUC |
| | | | CUGCACAGGCCGAGGAGGCCCCCGAAGUUGUAGCGACACCGUCACC |
| | | | AUCUACAGCUGAUAACACCUCGCUUGAUGUCACAGACAUCUCACUG |
| | | | GAUAUGGAUGACAGUAGCGAAGGCUCACUUUUUUCGAGCUUUAGCG |
| | | | GAUCGGACAACUCUAUUACUAGUAUGGACAGUUGGUCGUCAGGACC |
| | | | UAGUUCACUAGAGAUAGUAGACCGAAGGCAGGUGGUGGUGGCUGAC |
| | | | GUUCAUGCCGUCCAAGAGCCUGCCCCUAUUCCACCGCCAAGGCUAA |
| | | | AGAAGAUGGCCCGCCUGGCAGCGGCAAGAAAAGAGCCCACUCCACC |
| | | | GGCAAGCAAUAGCUCUGAGUCCCUCCACCUCUCUUUUGGUGGGGUA |
| | | | UCCAUGUCCCUCGGAUCAAUUUUCGACGGAGAGACGGCCCGCCAGG |
| | | | CAGCGGUACAACCCCUGGCAACAGGCCCCACGGAUGUGCCUAUGUC |
| | | | UUUCGGAUCGUUUUCCGACGGAGAGAUUGAUGAGCUGAGCCGCAGA |
| | | | GUAACUGAGUCCGAACCCGUCCUGUUUGGAUCAUUUGAACCGGGCG |
| | | | AAGUGAACUCAAUUAUAUCGUCCCGAUCAGCCGUAUCUUUUCCUCU |
| | | | ACGCAAGCAGAGACGUAGACGCAGGAGCAGGAGGACUGAAUACUGA |
| | | | CUAACCGGGGUAGGUGGGUACAUAUUUUCGACGGACACAGGCCCUG |
| | | | GGCACUUGCAAAAGAAGUCCGUUCUGCAGAACCAGCUUACAGAACC |
| | | | GACCUUGGAGCGCAAUGUCCUGGAAAGAAUUCAUGCCCCGGUGCUC |
| | | | GACACGUCGAAAGAGGAACAACUCAAACUCAGGUACCAGAUGAUGC |
| | | | CCACCGAAGCCAACAAAAGUAGGUACCAGUCUCGUAAAGUAGAAAA |
| | | | UCAGAAAGCCAUAACCACUGAGCGACUACUGUCAGGACUACGACUG |
| | | | UAUAACUCUGCCACAGAUCAGCCAGAAUGCUAUAAGAUCACCUAUC |
| | | | CGAAACCAUUGUACUCCAGUAGCGUACCGGCGAACUACUCCGAUCC |
| | | | ACAGUUCGCUGUAGCUGUCUGUAACAACUAUCUGCAUGAGAACUAU |
| | | | CCGACAGUAGCAUCUUAUCAGAUUACUGACGAGUACGAUGCUUACU |
| | | | UGGAUAUGGUAGACGGGACAGUCGCCUGCCUGGACACUGCAACCUU |
| | | | CUGCCCCGCUAAGCUUAGAAGUUACCCGAAAAAACAUGAGUAUAGA |
| | | | GCCCCGAAUAUCCGCAGUGCGGUUCCAUCAGCGAUGCAGAACACGC |
| | | | UACAAAAUGUGCUCAUUGCCGCAACUAAAAGAAAUUGCAACGUCAC |
| | | | GCAGAUGCGUGAACUGCCAACACUGGACUCAGCGACAUUCAAUGUC |
| | | | GAAUGCUUUCGAAAAUAUGCAUGUAAUGACGAGUAUUGGGAGGAGU |
| | | | UCGCUCGGAAGCCAAUUAGGAUUACCACUGAGUUUGUCACCGCAUA |
| | | | UGUAGCUAGACUGAAAGGCCCUAAGGCCGCCGCACUAUUUGCAAAG |
| | | | ACGUAUAAUUUGGUCCCAUUGCAAGAAGUGCCUAUGGAUAGAUUCG |
| | | | UCAUGGACAUGAAAAGAGACGUGAAAGUUACACCAGGCACGAAACA |
| | | | CACAGAAGAAAGACCGAAAGUACAAGUGAUACAAGCCGCAGAACCC |
| | | | CUGGCGACUGCUUACUUAUGCGGGAUUCACCGGGAAUUAGUGCGUA |
| | | | GGCUUACGGCCGUCUUGCUUCCAAACAUUCACACGCUUUUUGACAU |
| | | | GUCGGCGGAGGAUUUUGAUGCAAUCAUAGCAGAACACUUCAAGCAA |
| | | | GGCGACCCGGUACUGGAGACGGAUAUCGCAUCAUUCGACAAAAGCC |
| | | | AAGACGACGCUAUGGCGUUAACCGGUCUGAUGAUCUUGGAGGACCU |
| | | | GGGUGUGGAUCAACCACUACUCGACUUGAUCGAGUGCGCCUUUGGA |
| | | | GAAAUAUCAUCCACCCAUCUACCUACGGGUACUCGUUUUAAAUUCG |
| | | | GGGCGAUGAUGAAAUCCGGAAUGUUCCUCACACUUUUUGUCAACAC |
| | | | AGUUUUGAAUGUCGUUAUCGCCAGCAGAGUACUAGAGGAGCGGCUU |
| | | | AAAACGUCCAGAUGUGCAGCGUUCAUUGGCGACGACAACAUCAUAC |
| | | | AUGGAGUAGUAUCUGACAAAGAAAUGGCUGAGAGGUGCGCCACCUG |
| | | | GCUCAACAUGGAGGUUAAGAUCAUCGACGCAGUCAUCGGUGAGAGA |
| | | | CCACCUUACUUCUGCGGCGGAUUUAUCUUGCAAGAUUCGGUUACUU |
| | | | CCACAGCGUGCCGCGUGGCGGAUCCCCUGAAAAGGCUGUUUAAGUU |
| | | | GGGUAAACCGCUCCCAGCCGACGACGAGCAAGACGAAGACAGAAGA |
| | | | CGCGCUCUGCUAGAUGAAACAAAGGCGUGGUUUAGAGUAGGUAUAA |
| | | | CAGGCACUUUAGCAGUGGCCGUGACGACCCGGUAUGAGGUAGACAA |
| | | | UAUUACACCUGUCCUACUGGCAUUGAGAACUUUUGCCCAGAGCAAA |
| | | | AGAGCAUUCCAAGCCAUCAGAGGGGAAAUAAAGCAUCUCUACGGUG |
| | | | GUCCUAAAUAGUCAGCAUAGUACAUUUCAUCUGACUAAUACUACAA |
| | | | CACCACCACCAUGGAAGAUGCCAAAAACAUUAAGAAGGGCCCAGCG |
| | | | CCAUUCUACCCACUCGAAGACGGGACCGCCGGCGAGCAGCUGCACA |
| | | | AAGCCAUGAAGCGCUACGCCCUGGUGCCCGGCACCAUCGCCUUUAC |
| | | | CGACGCACAUAUCGAGGUGGACAUUACCUACGCCGAGUACUUCGAG |
| | | | AUGAGCGUUCGGCUGGCAGAAGCUAUGAAGCGCUAUGGGCUGAAUA |
| | | | CAAACCAUCGGAUCGUGGUGUGCAGCGAGAAUAGCUUGCAGUUCUU |
| | | | CAUGCCCGUGUUGGGUGCCCUGUUCAUCGGUGUGGCUGUGGCCCCA |
| | | | GCUAACGACAUCUACAACGAGCGCGAGCUGCUGAACAGCAUGGGCA |
| | | | UCAGCCAGCCCACCGUCGUAUUCGUGAGCAAGAAAGGGCUGCAAAA |
| | | | GAUCCUCAACGUGCAAAAGAAGCUACCGAUCAUACAAAAGAUCAUC |
| | | | AUCAUGGAUAGCAAGACCGACUACCAGGGCUUCCAAAGCAUGUACA |
| | | | CCUUCGUGACUUCCCAUUUGCCACCCGGCUUCAACGAGUACGACUU |
| | | | CGUGCCCGAGAGCUUCGACCGGGACAAAACCAUCGCCCUGAUCAUG |
| | | | AACAGUAGUGGCAGUACCGGAUUGCCCAAGGGCGUAGCCCUACCGC |
| | | | ACCGCACCGCUUGUGUCCGAUUCAGUCAUGCCCGCGACCCCAUCUU |
| | | | CGGCAACCAGAUCAUCCCCGACACCGCUAUCCUCAGCGUGGUGCCA |
| | | | UUUCACCACGGCUUCGGCAUGUUCACCACGCUGGGCUACUUGAUCU |
| | | | GCGGCUUUCGGGUCGUGCUCAUGUACCGCUUCGAGGAGGAGCUAUU |
| | | | CUUGCGCAGCUUGCAAGACUAUAAGAUUCAAUCUGCCCUGCUGGUG |
| | | | CCCACACUAUUUAGCUUCUUCGCUAAGAGCACUCUCAUCGACAAGU |
| | | | ACGACCUAAGCAACUUGCACGAGAUCGCCAGCGGCGGGGCGCCGCU |
| | | | CAGCAAGGAGGUAGGUGAGGCCGUGGCCAAACGCUUCCACCUACCA |
| | | | GGCAUCCGACAGGGCUACGGCCUGACAGAAACAACCAGCGCCAUUC |
| | | | UGAUCACCCCCGAAGGGGACGACAAGCCUGGCGCAGUAGGCAAGGU |
| | | | GGUGCCCUUCUUCGAGGCUAAGGUGGUGGACUUGGACACCGGUAAG |
| | | | ACACUGGGUGUGAACCAGCGCGGCGAGCUGUGCGUCCGUGGCCCCA |
| | | | UGAUCAUGAGCGGCUACGUUAACAACCCCGAGGCUACAAACGCUCU |
| | | | CAUCGACAAGGACGGCUGGCUGCACAGCGGCGACAUCGCCUACUGG |
| | | | GACGAGGACGAGCACUUCUUCAUCGUGGACCGGCUGAAGUCCCUGA |
| | | | UCAAAUACAAGGGCUACCAGGUAGCCCCAGCCGAACUGGAGAGCAU |
| | | | CCUGCUGCAACACCCCAACAUCUUCGACGCCGGGGUCGCCGGCCUG |
| | | | CCCGACGACGAUGCCGGCGAGCUGCCCGCCGCAGUCGUCGUGCUGG |
| | | | AACACGGUAAAACCAUGACCGAGAAGGAGAUCGUGGACUAUGUGGC |
| | | | CAGCCAGGUUACAACCGCCAAGAAGCUGCGCGGUGGUGUUGUGUUC |
| | | | GUGGACGAGGUGCCUAAAGGACUGACCGGCAAGUUGGACGCCCGCA |
| | | | AGAUCCGCGAGAUUCUCAUUAAGGCCAAGAAGGGCGGCAAGAUCGC |
| | | | CGUGUAAACGCGUGCUAGACCAUGGAUCCUAGACGCUACGCCCCAA |
| | | | UGAUCCGACCAGCAAAACUCGAUGUACUUCCGAGGAACUGAUGUGC |
| | | | AUAAUGCAUCAGGCUGGUACAUUAGAUCCCCGCUUACCGCGGGCAA |
| | | | UAUAGCAACACUAAAAACUCGAUGUACUUCCGAGGAAGCGCAGUGC |
| | | | AUAAUGCUGCGCAGUGUUGCCACAUAACCACUAUAUUAACCAUUUA |
| | | | UCUAGCGGACGCCAAAAACUCAAUGUAUUUCUGAGGAAGCGUGGUG |
| | | | CAUAAUGCCACGCAGCGUCUGCAUAACUUUUAUUAUUUCUUUUAUU |
| | | | AAUCAACAAAAUUUUGUUUUUAACAUUUCAAAAAAAAAAAAAAAAA |
| | | | AAAAAAAAUCUAGAAAAAAAAAAAAAAAAAAAAAAAAAAAAAAAAA |
| | | | AAAAAAAAAAAAAAAAAAAAAAAAAAAAAAAAAAAAAAAAAAAAAA |
| | | | AAAAAAAAAAAAAAAAAAAAA |
| 1782 | mRNA | 1782 | AGGAAACUUAAGUCAACACAACAUAUACAAAACAAACGAAUCUCAA |
| (SEQ | Fluc | | GCAAUCAAGCAUUCUACUUCUAUUGCAGCAAUUUAAAUCAUUUCUU |
| ID | | | UUAAAGCAAAAGCAAUUUUCUGAAAAUUUUCACCAUUUACGAACGA |
| NO: 102) | | | UAGCCAUGGAAGAUGCCAAAAACAUUAAGAAGGGCCCAGCGCCAUU |
| | | | CUACCCACUCGAAGACGGGACCGCCGGCGAGCAGCUGCACAAAGCC |
| | | | AUGAAGCGCUACGCCCUGGUGCCCGGCACCAUCGCCUUUACCGACG |
| | | | CACAUAUCGAGGUGGACAUUACCUACGCCGAGUACUUCGAGAUGAG |
| | | | CGUUCGGCUGGCAGAAGCUAUGAAGCGCUAUGGGCUGAAUACAAAC |
| | | | CAUCGGAUCGUGGUGUGCAGCGAGAAUAGCUUGCAGUUCUUCAUGC |
| | | | CCGUGUUGGGUGCCCUGUUCAUCGGUGUGGCUGUGGCCCCAGCUAA |
| | | | CGACAUCUACAACGAGCGCGAGCUGCUGAACAGCAUGGGCAUCAGC |
| | | | CAGCCCACCGUCGUAUUCGUGAGCAAGAAAGGGCUGCAAAAGAUCC |
| | | | UCAACGUGCAAAAGAAGCUACCGAUCAUACAAAAGAUCAUCAUCAU |
| | | | GGAUAGCAAGACCGACUACCAGGGCUUCCAAAGCAUGUACACCUUC |
| | | | GUGACUUCCCAUUUGCCACCCGGCUUCAACGAGUACGACUUCGUGC |
| | | | CCGAGAGCUUCGACCGGGACAAAACCAUCGCCCUGAUCAUGAACAG |
| | | | UAGUGGCAGUACCGGAUUGCCCAAGGGCGUAGCCCUACCGCACCGC |
| | | | ACCGCUUGUGUCCGAUUCAGUCAUGCCCGCGACCCCAUCUUCGGCA |
| | | | ACCAGAUCAUCCCCGACACCGCUAUCCUCAGCGUGGUGCCAUUUCA |
| | | | CCACGGCUUCGGCAUGUUCACCACGCUGGGCUACUUGAUCUGCGGC |
| | | | UUUCGGGUCGUGCUCAUGUACCGCUUCGAGGAGGAGCUAUUCUUGC |
| | | | GCAGCUUGCAAGACUAUAAGAUUCAAUCUGCCCUGCUGGUGCCCAC |
| | | | ACUAUUUAGCUUCUUCGCUAAGAGCACUCUCAUCGACAAGUACGAC |
| | | | CUAAGCAACUUGCACGAGAUCGCCAGCGGCGGGGCGCCGCUCAGCA |
| | | | AGGAGGUAGGUGAGGCCGUGGCCAAACGCUUCCACCUACCAGGCAU |
| | | | CCGACAGGGCUACGGCCUGACAGAAACAACCAGCGCCAUUCUGAUC |
| | | | ACCCCCGAAGGGGACGACAAGCCUGGCGCAGUAGGCAAGGUGGUGC |
| | | | CCUUCUUCGAGGCUAAGGUGGUGGACUUGGACACCGGUAAGACACU |
| | | | GGGUGUGAACCAGCGCGGCGAGCUGUGCGUCCGUGGCCCCAUGAUC |
| | | | AUGAGCGGCUACGUUAACAACCCCGAGGCUACAAACGCUCUCAUCG |
| | | | ACAAGGACGGCUGGCUGCACAGCGGCGACAUCGCCUACUGGGACGA |
| | | | GGACGAGCACUUCUUCAUCGUGGACCGGCUGAAGUCCCUGAUCAAA |
| | | | UACAAGGGCUACCAGGUAGCCCCAGCCGAACUGGAGAGCAUCCUGC |
| | | | UGCAACACCCCAACAUCUUCGACGCCGGGGUCGCCGGCCUGCCCGA |
| | | | CGACGAUGCCGGCGAGCUGCCCGCCGCAGUCGUCGUGCUGGAACAC |
| | | | GGUAAAACCAUGACCGAGAAGGAGAUCGUGGACUAUGUGGCCAGCC |
| | | | AGGUUACAACCGCCAAGAAGCUGCGCGGUGGUGUUGUGUUCGUGGA |
| | | | CGAGGUGCCUAAAGGACUGACCGGCAAGUUGGACGCCCGCAAGAUC |
| | | | CGCGAGAUUCUCAUUAAGGCCAAGAAGGGCGGCAAGAUCGCCGUGU |
| | | | AACUCGAGCUAGUGACUGACUAGGAUCUGGUUACCACUAAACCAGC |
| | | | CUCAAGAACACCCGAAUGGAGUCUCUAAGCUACAUAAUACCAACUU |
| | | | ACACUUACAAAAUGUUGUCCCCCAAAAUGUAGCCAUUCGUAUCUGC |
| | | | UCCUAAUAAAAAGAAAGUUUCUUCACAUUCUAGAAAAAAAAAAAAA |
| | | | AAAAAAAAAAAAAAAAAAAAAAAAAAAAAAAAAAAAAAAAAAAAAA |
| | | | AAAAAAAAAAAAAAAAAAAAAAAAAAAAAAAAAAAAAAAAAAAAAA |
| | | | AAAAAAAAAAAAAAAA |
| 2847 | STARR^{TM} | 2847 | AUGGGCGGCGCAUGAGAGAAGCCCAGACCAAUUACCUACCCAAAAU |
| (SEQ | KRAS | | GGAGAAAGUUCACGUUGACAUCGAGGAAGACAGCCCAUUCCUCAGA |
| ID | wt | | GCUUUGCAGCGGAGCUUCCCGCAGUUUGAGGUAGAAGCCAAGCAGG |
| NO: 103) | | | UCACUGAUAAUGACCAUGCUAAUGCCAGAGCGUUUUCGCAUCUGGC |
| | | | UUCAAAACUGAUCGAAACGGAGGUGGACCCAUCCGACACGAUCCUU |
| | | | GACAUUGGAAGUGCGCCCGCCCGCAGAAUGUAUUCUAAGCACAAGU |
| | | | AUCAUUGUAUCUGUCCGAUGAGAUGUGCGGAAGAUCCGGACAGAUU |
| | | | GUAUAAGUAUGCAACUAAGCUGAAGAAAAACUGUAAGGAAAUAACU |
| | | | GAUAAGGAAUUGGACAAGAAAAUGAAGGAGCUGGCCGCCGUCAUGA |
| | | | GCGACCCUGACCUGGAAACUGAGACUAUGUGCCUCCACGACGACGA |
| | | | GUCGUGUCGCUACGAAGGGCAAGUCGCUGUUUACCAGGAUGUAUAC |
| | | | GCCGUCGACGGCCCCACCAGCCUGUACCACCAGGCCAACAAGGGCG |
| | | | UGAGGGUGGCCUACUGGAUCGGCUUCGACACCACACCCUUCAUGUU |
| | | | CAAGAACCUGGCCGGCGCCUACCCCAGCUACAGCACCAACUGGGCC |
| | | | GACGAGACCGUGCUGACCGCCAGGAACAUCGGCCUGUGCAGCAGCG |
| | | | ACGUGAUGGAGAGGAGCCGGAGAGGCAUGAGCAUCCUGAGGAAGAA |
| | | | AUACCUGAAGCCCAGCAACAACGUGCUGUUCAGCGUGGGCAGCACC |
| | | | AUCUACCACGAGAAGAGGGACCUGCUCAGGAGCUGGCACCUGCCCA |
| | | | GCGUGUUCCACCUGAGGGGCAAGCAGAACUACACCUGCAGGUGCGA |
| | | | GACCAUCGUGAGCUGCGACGGCUACGUGGUGAAGAGGAUCGCCAUC |
| | | | AGCCCCGGCCUGUACGGCAAGCCCAGCGGCUACGCCGCUACAAUGC |
| | | | ACAGGGAGGGCUUCCUGUGCUGCAAGGUGACCGACACCCUGAACGG |
| | | | CGAGAGGGUGAGCUUCCCCGUGUGCACCUACGUGCCCGCCACCCUG |
| | | | UGCGACCAGAUGACCGGCAUCCUGGCCACCGACGUGAGCGCCGACG |
| | | | ACGCCCAGAAGCUGCUCGUGGGCCUGAACCAGAGGAUCGUGGUCAA |
| | | | CGGCAGGACCCAGAGGAACACCAACACAAUGAAGAACUACCUGCUG |
| | | | CCCGUGGUGGCCCAGGCUUUCGCCAGGUGGGCCAAGGAGUACAAGG |
| | | | AGGACCAGGAAGACGAGAGGCCCCUGGGCCUGAGGGACAGGCAGCU |
| | | | GGUGAUGGGCUGCUGCUGGGCCUUCAGGCGGCACAAGAUCACCAGC |
| | | | AUCUACAAGAGGCCCGACACCCAGACCAUCAUCAAGGUGAACAGCG |
| | | | ACUUCCACAGCUUCGUGCUGCCCAGGAUCGGCAGCAACACCCUGGA |
| | | | GAUCGGCCUGAGGACCCGGAUCAGGAAGAUGCUGGAGGAACACAAG |
| | | | GAGCCCAGCCCACUGAUCACCGCCGAGGACGUGCAGGAGGCCAAGU |
| | | | GCGCUGCCGACGAGGCCAAGGAGGUGAGGGAGGCCGAGGAACUGAG |
| | | | GGCCGCCCUGCCACCCCUGGCUGCCGACGUGGAGGAACCCACCCUG |
| | | | GAAGCCGACGUGGACCUGAUGCUGCAGGAGGCCGGCGCCGGAAGCG |
| | | | UGGAGACACCCAGGGGCCUGAUCAAGGUGACCAGCUACGACGGCGA |
| | | | GGACAAGAUCGGCAGCUACGCCGUGCUGAGCCCACAGGCCGUGCUG |
| | | | AAGUCCGAGAAGCUGAGCUGCAUCCACCCACUGGCCGAGCAGGUGA |
| | | | UCGUGAUCACCCACAGCGGCAGGAAGGGCAGGUACGCCGUGGAGCC |
| | | | CUACCACGGCAAGGUGGUCGUGCCCGAGGGCCACGCCAUCCCCGUG |
| | | | CAGGACUUCCAGGCCCUGAGCGAGAGCGCCACCAUCGUGUACAACG |
| | | | AGAGGGAGUUCGUGAACAGGUACCUGCACCAUAUCGCCACCCACGG |
| | | | CGGAGCCCUGAACACCGACGAGGAAUACUACAAGACCGUGAAGCCC |
| | | | AGCGAGCACGACGGCGAGUACCUGUACGACAUCGACAGGAAGCAGU |
| | | | GCGUGAAGAAAGAGCUGGUGACCGGCCUGGGACUGACCGGCGAGCU |
| | | | GGUGGACCCACCCUUCCACGAGUUCGCCUACGAGAGCCUGAGGACC |
| | | | AGACCCGCCGCUCCCUACCAGGUGCCCACCAUCGGCGUGUACGGCG |
| | | | UGCCCGGCAGCGGAAAGAGCGGCAUCAUCAAGAGCGCCGUGACCAA |
| | | | GAAAGACCUGGUGGUCAGCGCCAAGAAAGAGAACUGCGCCGAGAUC |
| | | | AUCAGGGACGUGAAGAAGAUGAAAGGCCUGGACGUGAACGCGCGCA |
| | | | CCGUGGACAGCGUGCUGCUGAACGGCUGCAAGCACCCCGUGGAGAC |
| | | | CCUGUACAUCGACGAGGCCUUCGCUUGCCACGCCGGCACCCUGAGG |
| | | | GCCCUGAUCGCCAUCAUCAGGCCCAAGAAAGCCGUGCUGUGCGGCG |
| | | | ACCCCAAGCAGUGCGGCUUCUUCAACAUGAUGUGCCUGAAGGUGCA |
| | | | CUUCAACCACGAGAUCUGCACCCAGGUGUUCCACAAGAGCAUCAGC |
| | | | AGGCGGUGCACCAAGAGCGUGACCAGCGUCGUGAGCACCCUGUUCU |
| | | | ACGACAAGAAAAUGAGGACCACCAACCCCAAGGAGACCAAAAUCGU |
| | | | GAUCGACACCACAGGCAGCACCAAGCCCAAGCAGGACGACCUGAUC |
| | | | CUGACCUGCUUCAGGGGCUGGGUGAAGCAGCUGCAGAUCGACUACA |
| | | | AGGGCAACGAGAUCAUGACCGCCGCUGCCAGCCAGGGCCUGACCAG |
| | | | GAAGGGCGUGUACGCCGUGAGGUACAAGGUGAACGAGAACCCACUG |
| | | | UACGCUCCCACCAGCGAGCACGUGAACGUGCUGCUGACCAGGACCG |
| | | | AGGACAGGAUCGUGUGGAAGACCCUGGCCGGCGACCCCUGGAUCAA |
| | | | GACCCUGACCGCCAAGUACCCCGGCAACUUCACCGCCACCAUCGAA |
| | | | GAGUGGCAGGCCGAGCACGACGCCAUCAUGAGGCACAUCCUGGAGA |
| | | | GGCCCGACCCCACCGACGUGUUCCAGAACAAGGCCAACGUGUGCUG |
| | | | GGCCAAGGCCCUGGUGCCCGUGCUGAAGACCGCCGGCAUCGACAUG |
| | | | ACCACAGAGCAGUGGAACACCGUGGACUACUUCGAGACCGACAAGG |
| | | | CCCACAGCGCCGAGAUCGUGCUGAACCAGCUGUGCGUGAGGUUCUU |
| | | | CGGCCUGGACCUGGACAGCGGCCUGUUCAGCGCCCCCACCGUGCCA |
| | | | CUGAGCAUCAGGAACAACCACUGGGACAACAGCCCCAGCCCAAACA |
| | | | UGUACGGCCUGAACAAGGAGGUGGUCAGGCAGCUGAGCAGGCGGUA |
| | | | CCCACAGCUGCCCAGGGCCGUGGCCACCGGCAGGGUGUACGACAUG |
| | | | AACACCGGCACCCUGAGGAACUACGACCCCAGGAUCAACCUGGUGC |
| | | | CCGUGAACAGGCGGCUGCCCCACGCCCUGGUGCUGCACCACAACGA |
| | | | GCACCCACAGAGCGACUUCAGCUCCUUCGUGAGCAAGCUGAAAGGC |
| | | | AGGACCGUGCUGGUCGUGGGCGAGAAGCUGAGCGUGCCCGGCAAGA |
| | | | UGGUGGACUGGCUGAGCGACAGGCCCGAGGCCACCUUCCGGGCCAG |
| | | | GCUGGACCUCGGCAUCCCCGGCGACGUGCCCAAGUACGACAUCAUC |
| | | | UUCGUGAACGUCAGGACCCCAUACAAGUACCACCAUUACCAGCAGU |
| | | | GCGAGGACCACGCCAUCAAGCUGAGCAUGCUGACCAAGAAGGCCUG |
| | | | CCUGCACCUGAACCCCGGAGGCACCUGCGUGAGCAUCGGCUACGGC |
| | | | UACGCCGACAGGGCCAGCGAGAGCAUCAUUGGCGCCAUCGCCAGGC |
| | | | UGUUCAAGUUCAGCAGGGUGUGCAAACCCAAGAGCAGCCUGGAGGA |
| | | | AACCGAGGUGCUGUUCGUGUUCAUCGGCUACGACCGGAAGGCCAGG |
| | | | ACCCACAACCCCUACAAGCUGAGCAGCACCCUGACAAACAUCUACA |
| | | | CCGGCAGCAGGCUGCACGAGGCCGGCUGCGCCCCCAGCUACCACGU |
| | | | GGUCAGGGGCGAUAUCGCCACCGCCACCGAGGGCGUGAUCAUCAAC |
| | | | GCUGCCAACAGCAAGGGCCAGCCCGGAGGCGGAGUGUGCGGCGCCC |
| | | | UGUACAAGAAGUUCCCCGAGAGCUUCGACCUGCAGCCCAUCGAGGU |
| | | | GGGCAAGGCCAGGCUGGUGAAGGGCGCCGCUAAGCACAUCAUCCAC |
| | | | GCCGUGGGCCCCAACUUCAACAAGGUGAGCGAGGUGGAAGGCGACA |
| | | | AGCAGCUGGCCGAAGCCUACGAGAGCAUCGCCAAGAUCGUGAACGA |
| | | | CAAUAACUACAAGAGCGUGGCCAUCCCACUGCUCAGCACCGGCAUC |
| | | | UUCAGCGGCAACAAGGACAGGCUGACCCAGAGCCUGAACCACCUGC |
| | | | UCACCGCCCUGGACACCACCGAUGCCGACGUGGCCAUCUACUGCAG |
| | | | GGACAAGAAGUGGGAGAUGACCCUGAAGGAGGCCGUGGCCAGGCGG |
| | | | GAGGCCGUGGAAGAGAUCUGCAUCAGCGACGACUCCAGCGUGACCG |
| | | | AGCCCGACGCCGAGCUGGUGAGGGUGCACCCCAAGAGCUCCCUGGC |
| | | | CGGCAGGAAGGGCUACAGCACCAGCGACGGCAAGACCUUCAGCUAC |
| | | | CUGGAGGGCACCAAGUUCCACCAGGCCGCUAAGGACAUCGCCGAGA |
| | | | UCAACGCUAUGUGGCCCGUGGCCACCGAGGCCAACGAGCAGGUGUG |
| | | | CAUGUACAUCCUGGGCGAGAGCAUGUCCAGCAUCAGGAGCAAGUGC |
| | | | CCCGUGGAGGAAAGCGAGGCCAGCACACCACCCAGCACCCUGCCCU |
| | | | GCCUGUGCAUCCACGCUAUGACACCCGAGAGGGUGCAGCGGCUGAA |
| | | | GGCCAGCAGGCCCGAGCAGAUCACCGUGUGCAGCUCCUUCCCACUG |
| | | | CCCAAGUACAGGAUCACCGGCGUGCAGAAGAUCCAGUGCAGCCAGC |
| | | | CCAUCCUGUUCAGCCCAAAGGUGCCCGCCUACAUCCACCCCAGGAA |
| | | | GUACCUGGUGGAGACCCCACCCGUGGACGAGACACCCGAGCCAAGC |
| | | | GCCGAGAACCAGAGCACCGAGGGCACACCCGAGCAGCCACCCCUGA |
| | | | UCACCGAGGACGAGACAAGGACCCGGACCCCAGAGCCCAUCAUUAU |
| | | | CGAGGAAGAGGAAGAGGACAGCAUCAGCCUGCUGAGCGACGGCCCC |
| | | | ACCCACCAGGUGCUGCAGGUGGAGGCCGACAUCCACGGCCCACCCA |
| | | | GCGUGUCCAGCUCCAGCUGGAGCAUCCCACACGCCAGCGACUUCGA |
| | | | CGUGGACAGCCUGAGCAUCCUGGACACCCUGGAGGGCGCCAGCGUG |
| | | | ACCUCCGGCGCCACCAGCGCCGAGACCAACAGCUACUUCGCCAAGA |
| | | | GCAUGGAGUUCCUGGCCAGGCCCGUGCCAGCUCCCAGGACCGUGUU |
| | | | CAGGAACCCACCCCACCCAGCUCCCAGGACCAGGACCCCAAGCCUG |
| | | | GCUCCCAGCAGGGCCUGCAGCAGGACCAGCCUGGUGAGCACCCCAC |
| | | | CCGGCGUGAACAGGGUGAUCACCAGGGAGGAACUGGAGGCCCUGAC |
| | | | ACCCAGCAGGACCCCCAGCAGGUCCGUGAGCAGGACUAGUCUGGUG |
| | | | UCCAACCCACCCGGCGUGAACAGGGUGAUCACCAGGGAGGAAUUCG |
| | | | AGGCCUUCGUGGCCCAGCAACAGAGACGGUUCGACGCCGGCGCCUA |
| | | | CAUCUUCAGCAGCGACACCGGCCAGGGACACCUGCAGCAAAAGAGC |
| | | | GUGAGGCAGACCGUGCUGAGCGAGGUGGUGCUGGAGAGGACCGAGC |
| | | | UGGAAAUCAGCUACGCCCCCAGGCUGGACCAGGAGAAGGAGGAACU |
| | | | GCUCAGGAAGAAACUGCAGCUGAACCCCACCCCAGCCAACAGGAGC |
| | | | AGGUACCAGAGCAGGAAGGUGGAGAACAUGAAGGCCAUCACCGCCA |
| | | | GGCGGAUCCUGCAGGGCCUGGGACACUACCUGAAGGCCGAGGGCAA |
| | | | GGUGGAGUGCUACAGGACCCUGCACCCCGUGCCACUGUACAGCUCC |
| | | | AGCGUGAACAGGGCCUUCUCCAGCCCCAAGGUGGCCGUGGAGGCCU |
| | | | GCAACGCUAUGCUGAAGGAGAACUUCCCCACCGUGGCCAGCUACUG |
| | | | CAUCAUCCCCGAGUACGACGCCUACCUGGACAUGGUGGACGGCGCC |
| | | | AGCUGCUGCCUGGACACCGCCAGCUUCUGCCCCGCCAAGCUGAGGA |
| | | | GCUUCCCCAAGAAACACAGCUACCUGGAGCCCACCAUCAGGAGCGC |
| | | | CGUGCCCAGCGCCAUCCAGAACACCCUGCAGAACGUGCUGGCCGCU |
| | | | GCCACCAAGAGGAACUGCAACGUGACCCAGAUGAGGGAGCUGCCCG |
| | | | UGCUGGACAGCGCUGCCUUCAACGUGGAGUGCUUCAAGAAAUACGC |
| | | | CUGCAACAACGAGUACUGGGAGACCUUCAAGGAGAACCCCAUCAGG |
| | | | CUGACCGAAGAGAACGUGGUGAACUACAUCACCAAGCUGAAGGGCC |
| | | | CCAAGGCCGCUGCCCUGUUCGCUAAGACCCACAACCUGAACAUGCU |
| | | | GCAGGACAUCCCAAUGGACAGGUUCGUGAUGGACCUGAAGAGGGAC |
| | | | GUGAAGGUGACACCCGGCACCAAGCACACCGAGGAGAGGCCCAAGG |
| | | | UGCAGGUGAUCCAGGCCGCUGACCCACUGGCCACCGCCUACCUGUG |
| | | | CGGCAUCCACAGGGAGCUGGUGAGGCGGCUGAACGCCGUGCUGCUG |
| | | | CCCAACAUCCACACCCUGUUCGACAUGAGCGCCGAGGACUUCGACG |
| | | | CCAUCAUCGCCGAGCACUUCCAGCCCGGCGACUGCGUGCUGGAGAC |
| | | | CGACAUCGCCAGCUUCGACAAGAGCGAGGAUGACGCUAUGGCCCUG |
| | | | ACCGCUCUGAUGAUCCUGGAGGACCUGGGCGUGGACGCCGAGCUGC |
| | | | UCACCCUGAUCGAGGCUGCCUUCGGCGAGAUCAGCUCCAUCCACCU |
| | | | GCCCACCAAGACCAAGUUCAAGUUCGGCGCUAUGAUGAAAAGCGGA |
| | | | AUGUUCCUGACCCUGUUCGUGAACACCGUGAUCAACAUUGUGAUCG |
| | | | CCAGCAGGGUGCUGCGGGAGAGGCUGACCGGCAGCCCCUGCGCUGC |
| | | | CUUCAUCGGCGACGACAACAUCGUGAAGGGCGUGAAAAGCGACAAG |
| | | | CUGAUGGCCGACAGGUGCGCCACCUGGCUGAACAUGGAGGUGAAGA |
| | | | UCAUCGACGCCGUGGUGGGCGAGAAGGCCCCCUACUUCUGCGGCGG |
| | | | AUUCAUCCUGUGCGACAGCGUGACCGGCACCGCCUGCAGGGUGGCC |
| | | | GACCCCCUGAAGAGGCUGUUCAAGCUGGGCAAGCCACUGGCCGCUG |
| | | | ACGAUGAGCACGACGAUGACAGGCGGAGGGCCCUGCACGAGGAAAG |
| | | | CACCAGGUGGAACAGGGUGGGCAUCCUGAGCGAGCUGUGCAAGGCC |
| | | | GUGGAGAGCAGGUACGAGACCGUGGGCACCAGCAUCAUCGUGAUGG |
| | | | CUAUGACCACACUGGCCAGCUCCGUCAAGAGCUUCUCCUACCUGAG |
| | | | GGGGGCCCCUAUAACUCUCUACGGCUAACCUGAAUGGACUACGACA |
| | | | UAGUCUAGUCCGCCAAGGCCGCCACCCAUGAAGUUGGUGGUUGUGG |
| | | | GGGCCGGGGGUGUUGGCAAAAGCGCCCUUACAAUUUGACUCGAGUA |
| | | | UGUUACGUGCAAAGGUGAUUGUCACCCCCCGAAAGACCAUAUUGUG |
| | | | ACACACCCUCAGUAUCACGCCCAAACAUUUACAGCCGCGGUGUCAA |
| | | | AAACCGCGUGGACGUGGUUAACAUCCCUGCUGGGAGGAUCAGCCGU |
| | | | AAUUAUUAUAAUUGGCUUGGUGCUGGCUACUAUUGUGGCCAUGUAC |
| | | | GUGCUGACCAACCAGAAACAUAAUUGAAUACAGCAGCAAUUGGCAA |
| | | | GCUGCUUACAUAGAACUCGCGGCGAUUGGCAUGCCGCCUUAAAAUU |
| | | | UUUAUUUUAUUUUUUCUUUUCUUUUCCGAAUCGGAUUUUGUUUUUA |
| | | | AUAUUUCAAAAAAAAAAAAAAAAAAAAAAAAAUCUAGAAAAAAAAA |
| | | | AAAAAAAAAAAAAAAAAAAAAAAAAAAAAAAAAAAAAAAAAAAAAA |
| | | | AAAAAAAAAAAAAAAAAAAAAAAAAAAAAAAAAAAAAAAAAAAAA |
| 2862 | SINV | 2862 | AUUGACGGCGUAGUACACACUAUUGAAUCAAACAGCCGACCAAUUG |
| (SEQ | empty | | CACUACCAUCACAAUGGAGAAGCCAGUAGUAAACGUAGACGUAGAC |
| ID | | | CCCCAGAGUCCGUUUGUCGUGCAACUGCAAAAAAGCUUCCCGCAAU |
| NO: 104) | | | UUGAGGUAGUAGCACAGCAGGUCACUCCAAAUGACCAUGCUAAUGC |
| | | | CAGAGCAUUUUCGCAUCUGGCCAGUAAACUAAUCGAGCUGGAGGUU |
| | | | CCUACCACAGCGACGAUCUUGGACAUAGGCAGCGCACCGGCUCGUA |
| | | | GAAUGUUUUCCGAGCACCAGUAUCAUUGUGUCUGCCCCAUGCGUAG |
| | | | UCCAGAAGACCCGGACCGCAUGAUGAAAUAUGCCAGUAAACUGGCG |
| | | | GAAAAAGCGUGCAAGAUUACAAACAAGAACUUGCAUGAGAAGAUUA |
| | | | AGGAUCUCCGGACCGUACUUGAUACGCCGGAUGCUGAAACACCAUC |
| | | | GCUCUGCUUUCACAACGAUGUUACCUGCAACAUGCGUGCCGAAUAU |
| | | | UCCGUCAUGCAGGACGUGUAUAUCAACGCUCCCGGAACUAUCUAUC |
| | | | AUCAGGCUAUGAAAGGCGUGCGGACCCUGUACUGGAUUGGCUUCGA |
| | | | CACCACCCAGUUCAUGUUCUCGGCUAUGGCAGGUUCGUACCCUGCG |
| | | | UACAACACCAACUGGGCCGACGAGAAAGUCCUUGAAGCGCGUAACA |
| | | | UCGGACUUUGCAGCACAAAGCUGAGUGAAGGUAGGACAGGAAAAUU |
| | | | GUCGAUAAUGAGGAAGAAGGAGUUGAAGCCCGGGUCGCGGGUUUAU |
| | | | UUCUCCGUAGGAUCGACACUUUAUCCAGAACACAGAGCCAGCUUGC |
| | | | AGAGCUGGCAUCUUCCAUCGGUGUUCCACUUGAAUGGAAAGCAGUC |
| | | | GUACACUUGCCGCUGUGAUACAGUGGUGAGUUGCGAAGGCUACGUA |
| | | | GUGAAGAAAAUCACCAUCAGUCCCGGGAUCACGGGAGAAACCGUGG |
| | | | GAUACGCGGUUACACACAAUAGCGAGGGCUUCUUGCUAUGCAAAGU |
| | | | UACUGACACAGUAAAAGGAGAACGGGUAUCGUUCCCUGUGUGCACG |
| | | | UACAUCCCGGCCACCAUAUGCGAUCAGAUGACUGGUAUAAUGGCCA |
| | | | CGGAUAUAUCACCUGACGAUGCACAAAAACUUCUGGUUGGGCUCAA |
| | | | CCAGCGAAUUGUCAUUAACGGUAGGACUAACAGGAACACCAACACC |
| | | | AUGCAAAAUUACCUUCUGCCGAUCAUAGCACAAGGGUUCAGCAAAU |
| | | | GGGCUAAGGAGCGCAAGGAUGAUCUUGAUAACGAGAAAAUGCUGGG |
| | | | UACUAGAGAACGCAAGCUUACGUAUGGCUGCUUGUGGGCGUUUCGC |
| | | | ACUAAGAAAGUACAUUCGUUUUAUCGCCCACCUGGAACGCAGACCU |
| | | | GCGUAAAAGUCCCAGCCUCUUUUAGCGCUUUUCCCAUGUCGUCCGU |
| | | | AUGGACGACCUCUUUGCCCAUGUCGCUGAGGCAGAAAUUGAAACUG |
| | | | GCAUUGCAACCAAAGAAGGAGGAAAAACUGCUGCAGGUCUCGGAGG |
| | | | AAUUAGUCAUGGAGGCCAAGGCUGCUUUUGAGGAUGCUCAGGAGGA |
| | | | AGCCAGAGCGGAGAAGCUCCGAGAAGCACUUCCACCAUUAGUGGCA |
| | | | GACAAAGGCAUCGAGGCAGCCGCAGAAGUUGUCUGCGAAGUGGAGG |
| | | | GGCUCCAGGCGGACAUCGGAGCAGCAUUAGUUGAAACCCCGCGCGG |
| | | | UCACGUAAGGAUAAUACCUCAAGCAAAUGACCGUAUGAUCGGACAG |
| | | | UAUAUCGUUGUCUCGCCAAACUCUGUGCUGAAGAAUGCCAAACUCG |
| | | | CACCAGCGCACCCGCUAGCAGAUCAGGUUAAGAUCAUAACACACUC |
| | | | CGGAAGAUCAGGAAGGUACGCGGUCGAACCAUACGACGCUAAAGUA |
| | | | CUGAUGCCAGCAGGAGGUGCCGUACCAUGGCCAGAAUUCCUAGCAC |
| | | | UGAGUGAGAGCGCCACGUUAGUGUACAACGAAAGAGAGUUUGUGAA |
| | | | CCGCAAACUAUACCACAUUGCCAUGCAUGGCCCCGCCAAGAAUACA |
| | | | GAAGAGGAGCAGUACAAGGUUACAAAGGCAGAGCUUGCAGAAACAG |
| | | | AGUACGUGUUUGACGUGGACAAGAAGCGUUGCGUUAAGAAGGAAGA |
| | | | AGCCUCAGGUCUGGUCCUCUCGGGAGAACUGACCAACCCUCCCUAU |
| | | | CAUGAGCUAGCUCUGGAGGGACUGAAGACCCGACCUGCGGUCCCGU |
| | | | ACAAGGUCGAAACAAUAGGAGUGAUAGGCACACCGGGGUCGGGCAA |
| | | | GUCAGCUAUUAUCAAGUCAACUGUCACGGCACGAGAUCUUGUUACC |
| | | | AGCGGAAAGAAAGAAAAUUGUCGCGAAAUUGAGGCCGACGUGCUAA |
| | | | GACUGAGGGGUAUGCAGAUUACGUCGAAGACAGUAGAUUCGGUUAU |
| | | | GCUCAACGGAUGCCACAAAGCCGUAGAAGUGCUGUACGUUGACGAA |
| | | | GCGUUCGCGUGCCACGCAGGAGCACUACUUGCCUUGAUUGCUAUCG |
| | | | UCAGGCCCCGCAAGAAGGUAGUACUAUGCGGAGACCCCAUGCAAUG |
| | | | CGGAUUCUUCAACAUGAUGCAACUAAAGGUACAUUUCAAUCACCCU |
| | | | GAAAAAGACAUAUGCACCAAGACAUUCUACAAGUAUAUCUCCCGGC |
| | | | GUUGCACACAGCCAGUUACAGCUAUUGUAUCGACACUGCAUUACGA |
| | | | UGGAAAGAUGAAAACCACGAACCCGUGCAAGAAGAACAUUGAAAUC |
| | | | GAUAUUACAGGGGCCACAAAGCCGAAGCCAGGGGAUAUCAUCCUGA |
| | | | CAUGUUUCCGCGGGUGGGUUAAGCAAUUGCAAAUCGACUAUCCCGG |
| | | | ACAUGAAGUAAUGACAGCCGCGGCCUCACAAGGGCUAACCAGAAAA |
| | | | GGAGUGUAUGCCGUCCGGCAAAAAGUCAAUGAAAACCCACUGUACG |
| | | | CGAUCACAUCAGAGCAUGUGAACGUGUUGCUCACCCGCACUGAGGA |
| | | | CAGGCUAGUGUGGAAAACCUUGCAGGGCGACCCAUGGAUUAAGCAG |
| | | | CUCACUAACAUACCUAAAGGAAACUUUCAGGCUACUAUAGAGGACU |
| | | | GGGAAGCUGAACACAAGGGAAUAAUUGCUGCAAUAAACAGCCCCAC |
| | | | UCCCCGUGCCAAUCCGUUCAGCUGCAAGACCAACGUUUGCUGGGCG |
| | | | AAAGCAUUGGAACCGAUACUAGCCACGGCCGGUAUCGUACUUACCG |
| | | | GUUGCCAGUGGAGCGAACUGUUCCCACAGUUUGCGGAUGACAAACC |
| | | | ACAUUCGGCCAUUUACGCCUUAGACGUAAUUUGCAUUAAGUUUUUC |
| | | | GGCAUGGACUUGACAAGCGGACUGUUUUCUAAACAGAGCAUCCCAC |
| | | | UAACGUACCAUCCCGCCGAUUCAGCGAGGCCGGUAGCUCAUUGGGA |
| | | | CAACAGCCCAGGAACCCGCAAGUAUGGGUACGAUCACGCCAUUGCC |
| | | | GCCGAACUCUCCCGUAGAUUUCCGGUGUUCCAGCUAGCUGGGAAGG |
| | | | GCACACAACUUGAUUUGCAGACGGGGAGAACCAGAGUUAUCUCUGC |
| | | | ACAGCAUAACCUGGUCCCGGUGAACCGCAAUCUUCCUCACGCCUUA |
| | | | GUCCCCGAGUACAAGGAGAAGCAACCCGGCCCGGUCGAAAAAUUCU |
| | | | UGAACCAGUUCAAACACCACUCAGUACUUGUGGUAUCAGAGGAAAA |
| | | | AAUUGAAGCUCCCCGUAAGAGAAUCGAAUGGAUCGCCCCGAUUGGC |
| | | | AUAGCCGGUGCAGAUAAGAACUACAACCUGGCUUUCGGGUUUCCGC |
| | | | CGCAGGCACGGUACGACCUGGUGUUCAUCAACAUUGGAACUAAAUA |
| | | | CAGAAACCACCACUUUCAGCAGUGCGAAGACCAUGCGGCGACCUUA |
| | | | AAAACCCUUUCGCGUUCGGCCCUGAAUUGCCUUAACCCAGGAGGCA |
| | | | CCCUCGUGGUGAAGUCCUAUGGCUACGCCGACCGCAACAGUGAGGA |
| | | | CGUAGUCACCGCUCUUGCCAGAAAGUUUGUCAGGGUGUCUGCAGCG |
| | | | AGACCAGAUUGUGUCUCAAGCAAUACAGAAAUGUACCUGAUUUUCC |
| | | | GACAACUAGACAACAGCCGUACACGGCAAUUCACCCCGCACCAUCU |
| | | | GAAUUGCGUGAUUUCGUCCGUGUAUGAGGGUACAAGAGAUGGAGUU |
| | | | GGAGCCGCGCCGUCAUACCGCACCAAAAGGGAGAAUAUUGCUGACU |
| | | | GUCAAGAGGAAGCAGUUGUCAACGCAGCCAAUCCGCUGGGUAGACC |
| | | | AGGCGAAGGAGUCUGCCGUGCCAUCUAUAAACGUUGGCCGACCAGU |
| | | | UUUACCGAUUCAGCCACGGAGACAGGCACCGCAAGAAUGACUGUGU |
| | | | GCCUAGGAAAGAAAGUGAUCCACGCGGUCGGCCCUGAUUUCCGGAA |
| | | | GCACCCAGAAGCAGAAGCCUUGAAAUUGCUACAAAACGCCUACCAU |
| | | | GCAGUGGCAGACUUAGUAAAUGAACAUAACAUCAAGUCUGUCGCCA |
| | | | UUCCACUGCUAUCUACAGGCAUUUACGCAGCCGGAAAAGACCGCCU |
| | | | UGAAGUAUCACUUAACUGCUUGACAACCGCGCUAGACAGAACUGAC |
| | | | GCGGACGUAACCAUCUAUUGCCUGGAUAAGAAGUGGAAGGAAAGAA |
| | | | UCGACGCGGCACUCCAACUUAAGGAGUCUGUAACAGAGCUGAAGGA |
| | | | UGAAGAUAUGGAGAUCGACGAUGAGUUAGUAUGGAUCCAUCCAGAC |
| | | | AGUUGCUUGAAGGGAAGAAAGGGAUUCAGUACUACAAAAGGAAAAU |
| | | | UGUAUUCGUACUUCGAAGGCACCAAAUUCCAUCAAGCAGCAAAAGA |
| | | | CAUGGCGGAGAUAAAGGUCCUGUUCCCUAAUGACCAGGAAAGUAAU |
| | | | GAACAACUGUGUGCCUACAUAUUGGGUGAGACCAUGGAAGCAAUCC |
| | | | GCGAAAAGUGCCCGGUCGACCAUAACCCGUCGUCUAGCCCGCCCAA |
| | | | AACGUUGCCGUGCCUUUGCAUGUAUGCCAUGACGCCAGAAAGGGUC |
| | | | CACAGACUUAGAAGCAAUAACGUCAAAGAAGUUACAGUAUGCUCCU |
| | | | CCACCCCCCUUCCUAAGCACAAAAUUAAGAAUGUUCAGAAGGUUCA |
| | | | GUGCACGAAAGUAGUCCUGUUUAAUCCGCACACUCCCGCAUUCGUU |
| | | | CCCGCCCGUAAGUACAUAGAAGUGCCAGAACAGCCUACCGCUCCUC |
| | | | CUGCACAGGCCGAGGAGGCCCCCGAAGUUGUAGCGACACCGUCACC |
| | | | AUCUACAGCUGAUAACACCUCGCUUGAUGUCACAGACAUCUCACUG |
| | | | GAUAUGGAUGACAGUAGCGAAGGCUCACUUUUUUCGAGCUUUAGCG |
| | | | GAUCGGACAACUCUAUUACUAGUAUGGACAGUUGGUCGUCAGGACC |
| | | | UAGUUCACUAGAGAUAGUAGACCGAAGGCAGGUGGUGGUGGCUGAC |
| | | | GUUCAUGCCGUCCAAGAGCCUGCCCCUAUUCCACCGCCAAGGCUAA |
| | | | AGAAGAUGGCCCGCCUGGCAGCGGCAAGAAAAGAGCCCACUCCACC |
| | | | GGCAAGCAAUAGCUCUGAGUCCCUCCACCUCUCUUUUGGUGGGGUA |
| | | | UCCAUGUCCCUCGGAUCAAUUUUCGACGGAGAGACGGCCCGCCAGG |
| | | | CAGCGGUACAACCCCUGGCAACAGGCCCCACGGAUGUGCCUAUGUC |
| | | | UUUCGGAUCGUUUUCCGACGGAGAGAUUGAUGAGCUGAGCCGCAGA |
| | | | GUAACUGAGUCCGAACCCGUCCUGUUUGGAUCAUUUGAACCGGGCG |
| | | | AAGUGAACUCAAUUAUAUCGUCCCGAUCAGCCGUAUCUUUUCCUCU |
| | | | ACGCAAGCAGAGACGUAGACGCAGGAGCAGGAGGACUGAAUACUGA |
| | | | CUAACCGGGGUAGGUGGGUACAUAUUUUCGACGGACACAGGCCCUG |
| | | | GGCACUUGCAAAAGAAGUCCGUUCUGCAGAACCAGCUUACAGAACC |
| | | | GACCUUGGAGCGCAAUGUCCUGGAAAGAAUUCAUGCCCCGGUGCUC |
| | | | GACACGUCGAAAGAGGAACAACUCAAACUCAGGUACCAGAUGAUGC |
| | | | CCACCGAAGCCAACAAAAGUAGGUACCAGUCUCGUAAAGUAGAAAA |
| | | | UCAGAAAGCCAUAACCACUGAGCGACUACUGUCAGGACUACGACUG |
| | | | UAUAACUCUGCCACAGAUCAGCCAGAAUGCUAUAAGAUCACCUAUC |
| | | | CGAAACCAUUGUACUCCAGUAGCGUACCGGCGAACUACUCCGAUCC |
| | | | ACAGUUCGCUGUAGCUGUCUGUAACAACUAUCUGCAUGAGAACUAU |
| | | | CCGACAGUAGCAUCUUAUCAGAUUACUGACGAGUACGAUGCUUACU |
| | | | UGGAUAUGGUAGACGGGACAGUCGCCUGCCUGGACACUGCAACCUU |
| | | | CUGCCCCGCUAAGCUUAGAAGUUACCCGAAAAAACAUGAGUAUAGA |
| | | | GCCCCGAAUAUCCGCAGUGCGGUUCCAUCAGCGAUGCAGAACACGC |
| | | | UACAAAAUGUGCUCAUUGCCGCAACUAAAAGAAAUUGCAACGUCAC |
| | | | GCAGAUGCGUGAACUGCCAACACUGGACUCAGCGACAUUCAAUGUC |
| | | | GAAUGCUUUCGAAAAUAUGCAUGUAAUGACGAGUAUUGGGAGGAGU |
| | | | UCGCUCGGAAGCCAAUUAGGAUUACCACUGAGUUUGUCACCGCAUA |
| | | | UGUAGCUAGACUGAAAGGCCCUAAGGCCGCCGCACUAUUUGCAAAG |
| | | | ACGUAUAAUUUGGUCCCAUUGCAAGAAGUGCCUAUGGAUAGAUUCG |
| | | | UCAUGGACAUGAAAAGAGACGUGAAAGUUACACCAGGCACGAAACA |
| | | | CACAGAAGAAAGACCGAAAGUACAAGUGAUACAAGCCGCAGAACCC |
| | | | CUGGCGACUGCUUACUUAUGCGGGAUUCACCGGGAAUUAGUGCGUA |
| | | | GGCUUACGGCCGUCUUGCUUCCAAACAUUCACACGCUUUUUGACAU |
| | | | GUCGGCGGAGGAUUUUGAUGCAAUCAUAGCAGAACACUUCAAGCAA |
| | | | GGCGACCCGGUACUGGAGACGGAUAUCGCAUCAUUCGACAAAAGCC |
| | | | AAGACGACGCUAUGGCGUUAACCGGUCUGAUGAUCUUGGAGGACCU |
| | | | GGGUGUGGAUCAACCACUACUCGACUUGAUCGAGUGCGCCUUUGGA |
| | | | GAAAUAUCAUCCACCCAUCUACCUACGGGUACUCGUUUUAAAUUCG |
| | | | GGGCGAUGAUGAAAUCCGGAAUGUUCCUCACACUUUUUGUCAACAC |
| | | | AGUUUUGAAUGUCGUUAUCGCCAGCAGAGUACUAGAGGAGCGGCUU |
| | | | AAAACGUCCAGAUGUGCAGCGUUCAUUGGCGACGACAACAUCAUAC |
| | | | AUGGAGUAGUAUCUGACAAAGAAAUGGCUGAGAGGUGCGCCACCUG |
| | | | GCUCAACAUGGAGGUUAAGAUCAUCGACGCAGUCAUCGGUGAGAGA |
| | | | CCACCUUACUUCUGCGGCGGAUUUAUCUUGCAAGAUUCGGUUACUU |
| | | | CCACAGCGUGCCGCGUGGCGGAUCCCCUGAAAAGGCUGUUUAAGUU |
| | | | GGGUAAACCGCUCCCAGCCGACGACGAGCAAGACGAAGACAGAAGA |
| | | | CGCGCUCUGCUAGAUGAAACAAAGGCGUGGUUUAGAGUAGGUAUAA |
| | | | CAGGCACUUUAGCAGUGGCCGUGACGACCCGGUAUGAGGUAGACAA |
| | | | UAUUACACCUGUCCUACUGGCAUUGAGAACUUUUGCCCAGAGCAAA |
| | | | AGAGCAUUCCAAGCCAUCAGAGGGGAAAUAAAGCAUCUCUACGGUG |
| | | | GUCCUAAAUAGUCAGCAUAGUACAUUUCAUCUGACUAAUACUACAA |
| | | | CACCACCACCACGCGUGCUAGACCAUGGAUCCUAGACGCUACGCCC |
| | | | CAAUGAUCCGACCAGCAAAACUCGAUGUACUUCCGAGGAACUGAUG |
| | | | UGCAUAAUGCAUCAGGCUGGUACAUUAGAUCCCCGCUUACCGCGGG |
| | | | CAAUAUAGCAACACUAAAAACUCGAUGUACUUCCGAGGAAGCGCAG |
| | | | UGCAUAAUGCUGCGCAGUGUUGCCACAUAACCACUAUAUUAACCAU |
| | | | UUAUCUAGCGGACGCCAAAAACUCAAUGUAUUUCUGAGGAAGCGUG |
| | | | GUGCAUAAUGCCACGCAGCGUCUGCAUAACUUUUAUUAUUUCUUUU |
| | | | AUUAAUCAACAAAAUUUUGUUUUUAACAUUUCAAAAAAAAAAAAAA |
| | | | AAAAAAAAAAAUCUAGAAAAAAAAAAAAAAAAAAAAAAAAAAAAAA |
| | | | AAAAAAAAAAAAAAAAAAAAAAAAAAAAAAAAAAAAAAAAAAAAAA |
| | | | AAAAAAAAAAAAAAAAAAAAAAAA |
| 3060 | STARR^{TM} | 3060 | AUGGGCGGCGCAUGAGAGAAGCCCAGACCAAUUACCUACCCAAAAU |
| (SEQ | gp70 | | GGAGAAAGUUCACGUUGACAUCGAGGAAGACAGCCCAUUCCUCAGA |
| ID | | | GCUUUGCAGCGGAGCUUCCCGCAGUUUGAGGUAGAAGCCAAGCAGG |
| NO: 105) | | | UCACUGAUAAUGACCAUGCUAAUGCCAGAGCGUUUUCGCAUCUGGC |
| | | | UUCAAAACUGAUCGAAACGGAGGUGGACCCAUCCGACACGAUCCUU |
| | | | GACAUUGGAAGUGCGCCCGCCCGCAGAAUGUAUUCUAAGCACAAGU |
| | | | AUCAUUGUAUCUGUCCGAUGAGAUGUGCGGAAGAUCCGGACAGAUU |
| | | | GUAUAAGUAUGCAACUAAGCUGAAGAAAAACUGUAAGGAAAUAACU |
| | | | GAUAAGGAAUUGGACAAGAAAAUGAAGGAGCUGGCCGCCGUCAUGA |
| | | | GCGACCCUGACCUGGAAACUGAGACUAUGUGCCUCCACGACGACGA |
| | | | GUCGUGUCGCUACGAAGGGCAAGUCGCUGUUUACCAGGAUGUAUAC |
| | | | GCCGUCGACGGCCCCACCAGCCUGUACCACCAGGCCAACAAGGGCG |
| | | | UGAGGGUGGCCUACUGGAUCGGCUUCGACACCACACCCUUCAUGUU |
| | | | CAAGAACCUGGCCGGCGCCUACCCCAGCUACAGCACCAACUGGGCC |
| | | | GACGAGACCGUGCUGACCGCCAGGAACAUCGGCCUGUGCAGCAGCG |
| | | | ACGUGAUGGAGAGGAGCCGGAGAGGCAUGAGCAUCCUGAGGAAGAA |
| | | | AUACCUGAAGCCCAGCAACAACGUGCUGUUCAGCGUGGGCAGCACC |
| | | | AUCUACCACGAGAAGAGGGACCUGCUCAGGAGCUGGCACCUGCCCA |
| | | | GCGUGUUCCACCUGAGGGGCAAGCAGAACUACACCUGCAGGUGCGA |
| | | | GACCAUCGUGAGCUGCGACGGCUACGUGGUGAAGAGGAUCGCCAUC |
| | | | AGCCCCGGCCUGUACGGCAAGCCCAGCGGCUACGCCGCUACAAUGC |
| | | | ACAGGGAGGGCUUCCUGUGCUGCAAGGUGACCGACACCCUGAACGG |
| | | | CGAGAGGGUGAGCUUCCCCGUGUGCACCUACGUGCCCGCCACCCUG |
| | | | UGCGACCAGAUGACCGGCAUCCUGGCCACCGACGUGAGCGCCGACG |
| | | | ACGCCCAGAAGCUGCUCGUGGGCCUGAACCAGAGGAUCGUGGUCAA |
| | | | CGGCAGGACCCAGAGGAACACCAACACAAUGAAGAACUACCUGCUG |
| | | | CCCGUGGUGGCCCAGGCUUUCGCCAGGUGGGCCAAGGAGUACAAGG |
| | | | AGGACCAGGAAGACGAGAGGCCCCUGGGCCUGAGGGACAGGCAGCU |
| | | | GGUGAUGGGCUGCUGCUGGGCCUUCAGGCGGCACAAGAUCACCAGC |
| | | | AUCUACAAGAGGCCCGACACCCAGACCAUCAUCAAGGUGAACAGCG |
| | | | ACUUCCACAGCUUCGUGCUGCCCAGGAUCGGCAGCAACACCCUGGA |
| | | | GAUCGGCCUGAGGACCCGGAUCAGGAAGAUGCUGGAGGAACACAAG |
| | | | GAGCCCAGCCCACUGAUCACCGCCGAGGACGUGCAGGAGGCCAAGU |
| | | | GCGCUGCCGACGAGGCCAAGGAGGUGAGGGAGGCCGAGGAACUGAG |
| | | | GGCCGCCCUGCCACCCCUGGCUGCCGACGUGGAGGAACCCACCCUG |
| | | | GAAGCCGACGUGGACCUGAUGCUGCAGGAGGCCGGCGCCGGAAGCG |
| | | | UGGAGACACCCAGGGGCCUGAUCAAGGUGACCAGCUACGACGGCGA |
| | | | GGACAAGAUCGGCAGCUACGCCGUGCUGAGCCCACAGGCCGUGCUG |
| | | | AAGUCCGAGAAGCUGAGCUGCAUCCACCCACUGGCCGAGCAGGUGA |
| | | | UCGUGAUCACCCACAGCGGCAGGAAGGGCAGGUACGCCGUGGAGCC |
| | | | CUACCACGGCAAGGUGGUCGUGCCCGAGGGCCACGCCAUCCCCGUG |
| | | | CAGGACUUCCAGGCCCUGAGCGAGAGCGCCACCAUCGUGUACAACG |
| | | | AGAGGGAGUUCGUGAACAGGUACCUGCACCAUAUCGCCACCCACGG |
| | | | CGGAGCCCUGAACACCGACGAGGAAUACUACAAGACCGUGAAGCCC |
| | | | AGCGAGCACGACGGCGAGUACCUGUACGACAUCGACAGGAAGCAGU |
| | | | GCGUGAAGAAAGAGCUGGUGACCGGCCUGGGACUGACCGGCGAGCU |
| | | | GGUGGACCCACCCUUCCACGAGUUCGCCUACGAGAGCCUGAGGACC |
| | | | AGACCCGCCGCUCCCUACCAGGUGCCCACCAUCGGCGUGUACGGCG |
| | | | UGCCCGGCAGCGGAAAGAGCGGCAUCAUCAAGAGCGCCGUGACCAA |
| | | | GAAAGACCUGGUGGUCAGCGCCAAGAAAGAGAACUGCGCCGAGAUC |
| | | | AUCAGGGACGUGAAGAAGAUGAAAGGCCUGGACGUGAACGCGCGCA |
| | | | CCGUGGACAGCGUGCUGCUGAACGGCUGCAAGCACCCCGUGGAGAC |
| | | | CCUGUACAUCGACGAGGCCUUCGCUUGCCACGCCGGCACCCUGAGG |
| | | | GCCCUGAUCGCCAUCAUCAGGCCCAAGAAAGCCGUGCUGUGCGGCG |
| | | | ACCCCAAGCAGUGCGGCUUCUUCAACAUGAUGUGCCUGAAGGUGCA |
| | | | CUUCAACCACGAGAUCUGCACCCAGGUGUUCCACAAGAGCAUCAGC |
| | | | AGGCGGUGCACCAAGAGCGUGACCAGCGUCGUGAGCACCCUGUUCU |
| | | | ACGACAAGAAAAUGAGGACCACCAACCCCAAGGAGACCAAAAUCGU |
| | | | GAUCGACACCACAGGCAGCACCAAGCCCAAGCAGGACGACCUGAUC |
| | | | CUGACCUGCUUCAGGGGCUGGGUGAAGCAGCUGCAGAUCGACUACA |
| | | | AGGGCAACGAGAUCAUGACCGCCGCUGCCAGCCAGGGCCUGACCAG |
| | | | GAAGGGCGUGUACGCCGUGAGGUACAAGGUGAACGAGAACCCACUG |
| | | | UACGCUCCCACCAGCGAGCACGUGAACGUGCUGCUGACCAGGACCG |
| | | | AGGACAGGAUCGUGUGGAAGACCCUGGCCGGCGACCCCUGGAUCAA |
| | | | GACCCUGACCGCCAAGUACCCCGGCAACUUCACCGCCACCAUCGAA |
| | | | GAGUGGCAGGCCGAGCACGACGCCAUCAUGAGGCACAUCCUGGAGA |
| | | | GGCCCGACCCCACCGACGUGUUCCAGAACAAGGCCAACGUGUGCUG |
| | | | GGCCAAGGCCCUGGUGCCCGUGCUGAAGACCGCCGGCAUCGACAUG |
| | | | ACCACAGAGCAGUGGAACACCGUGGACUACUUCGAGACCGACAAGG |
| | | | CCCACAGCGCCGAGAUCGUGCUGAACCAGCUGUGCGUGAGGUUCUU |
| | | | CGGCCUGGACCUGGACAGCGGCCUGUUCAGCGCCCCCACCGUGCCA |
| | | | CUGAGCAUCAGGAACAACCACUGGGACAACAGCCCCAGCCCAAACA |
| | | | UGUACGGCCUGAACAAGGAGGUGGUCAGGCAGCUGAGCAGGCGGUA |
| | | | CCCACAGCUGCCCAGGGCCGUGGCCACCGGCAGGGUGUACGACAUG |
| | | | AACACCGGCACCCUGAGGAACUACGACCCCAGGAUCAACCUGGUGC |
| | | | CCGUGAACAGGCGGCUGCCCCACGCCCUGGUGCUGCACCACAACGA |
| | | | GCACCCACAGAGCGACUUCAGCUCCUUCGUGAGCAAGCUGAAAGGC |
| | | | AGGACCGUGCUGGUCGUGGGCGAGAAGCUGAGCGUGCCCGGCAAGA |
| | | | UGGUGGACUGGCUGAGCGACAGGCCCGAGGCCACCUUCCGGGCCAG |
| | | | GCUGGACCUCGGCAUCCCCGGCGACGUGCCCAAGUACGACAUCAUC |
| | | | UUCGUGAACGUCAGGACCCCAUACAAGUACCACCAUUACCAGCAGU |
| | | | GCGAGGACCACGCCAUCAAGCUGAGCAUGCUGACCAAGAAGGCCUG |
| | | | CCUGCACCUGAACCCCGGAGGCACCUGCGUGAGCAUCGGCUACGGC |
| | | | UACGCCGACAGGGCCAGCGAGAGCAUCAUUGGCGCCAUCGCCAGGC |
| | | | UGUUCAAGUUCAGCAGGGUGUGCAAACCCAAGAGCAGCCUGGAGGA |
| | | | AACCGAGGUGCUGUUCGUGUUCAUCGGCUACGACCGGAAGGCCAGG |
| | | | ACCCACAACCCCUACAAGCUGAGCAGCACCCUGACAAACAUCUACA |
| | | | CCGGCAGCAGGCUGCACGAGGCCGGCUGCGCCCCCAGCUACCACGU |
| | | | GGUCAGGGGCGAUAUCGCCACCGCCACCGAGGGCGUGAUCAUCAAC |
| | | | GCUGCCAACAGCAAGGGCCAGCCCGGAGGCGGAGUGUGCGGCGCCC |
| | | | UGUACAAGAAGUUCCCCGAGAGCUUCGACCUGCAGCCCAUCGAGGU |
| | | | GGGCAAGGCCAGGCUGGUGAAGGGCGCCGCUAAGCACAUCAUCCAC |
| | | | GCCGUGGGCCCCAACUUCAACAAGGUGAGCGAGGUGGAAGGCGACA |
| | | | AGCAGCUGGCCGAAGCCUACGAGAGCAUCGCCAAGAUCGUGAACGA |
| | | | CAAUAACUACAAGAGCGUGGCCAUCCCACUGCUCAGCACCGGCAUC |
| | | | UUCAGCGGCAACAAGGACAGGCUGACCCAGAGCCUGAACCACCUGC |
| | | | UCACCGCCCUGGACACCACCGAUGCCGACGUGGCCAUCUACUGCAG |
| | | | GGACAAGAAGUGGGAGAUGACCCUGAAGGAGGCCGUGGCCAGGCGG |
| | | | GAGGCCGUGGAAGAGAUCUGCAUCAGCGACGACUCCAGCGUGACCG |
| | | | AGCCCGACGCCGAGCUGGUGAGGGUGCACCCCAAGAGCUCCCUGGC |
| | | | CGGCAGGAAGGGCUACAGCACCAGCGACGGCAAGACCUUCAGCUAC |
| | | | CUGGAGGGCACCAAGUUCCACCAGGCCGCUAAGGACAUCGCCGAGA |
| | | | UCAACGCUAUGUGGCCCGUGGCCACCGAGGCCAACGAGCAGGUGUG |
| | | | CAUGUACAUCCUGGGCGAGAGCAUGUCCAGCAUCAGGAGCAAGUGC |
| | | | CCCGUGGAGGAAAGCGAGGCCAGCACACCACCCAGCACCCUGCCCU |
| | | | GCCUGUGCAUCCACGCUAUGACACCCGAGAGGGUGCAGCGGCUGAA |
| | | | GGCCAGCAGGCCCGAGCAGAUCACCGUGUGCAGCUCCUUCCCACUG |
| | | | CCCAAGUACAGGAUCACCGGCGUGCAGAAGAUCCAGUGCAGCCAGC |
| | | | CCAUCCUGUUCAGCCCAAAGGUGCCCGCCUACAUCCACCCCAGGAA |
| | | | GUACCUGGUGGAGACCCCACCCGUGGACGAGACACCCGAGCCAAGC |
| | | | GCCGAGAACCAGAGCACCGAGGGCACACCCGAGCAGCCACCCCUGA |
| | | | UCACCGAGGACGAGACAAGGACCCGGACCCCAGAGCCCAUCAUUAU |
| | | | CGAGGAAGAGGAAGAGGACAGCAUCAGCCUGCUGAGCGACGGCCCC |
| | | | ACCCACCAGGUGCUGCAGGUGGAGGCCGACAUCCACGGCCCACCCA |
| | | | GCGUGUCCAGCUCCAGCUGGAGCAUCCCACACGCCAGCGACUUCGA |
| | | | CGUGGACAGCCUGAGCAUCCUGGACACCCUGGAGGGCGCCAGCGUG |
| | | | ACCUCCGGCGCCACCAGCGCCGAGACCAACAGCUACUUCGCCAAGA |
| | | | GCAUGGAGUUCCUGGCCAGGCCCGUGCCAGCUCCCAGGACCGUGUU |
| | | | CAGGAACCCACCCCACCCAGCUCCCAGGACCAGGACCCCAAGCCUG |
| | | | GCUCCCAGCAGGGCCUGCAGCAGGACCAGCCUGGUGAGCACCCCAC |
| | | | CCGGCGUGAACAGGGUGAUCACCAGGGAGGAACUGGAGGCCCUGAC |
| | | | ACCCAGCAGGACCCCCAGCAGGUCCGUGAGCAGGACUAGUCUGGUG |
| | | | UCCAACCCACCCGGCGUGAACAGGGUGAUCACCAGGGAGGAAUUCG |
| | | | AGGCCUUCGUGGCCCAGCAACAGAGACGGUUCGACGCCGGCGCCUA |
| | | | CAUCUUCAGCAGCGACACCGGCCAGGGACACCUGCAGCAAAAGAGC |
| | | | GUGAGGCAGACCGUGCUGAGCGAGGUGGUGCUGGAGAGGACCGAGC |
| | | | UGGAAAUCAGCUACGCCCCCAGGCUGGACCAGGAGAAGGAGGAACU |
| | | | GCUCAGGAAGAAACUGCAGCUGAACCCCACCCCAGCCAACAGGAGC |
| | | | AGGUACCAGAGCAGGAAGGUGGAGAACAUGAAGGCCAUCACCGCCA |
| | | | GGCGGAUCCUGCAGGGCCUGGGACACUACCUGAAGGCCGAGGGCAA |
| | | | GGUGGAGUGCUACAGGACCCUGCACCCCGUGCCACUGUACAGCUCC |
| | | | AGCGUGAACAGGGCCUUCUCCAGCCCCAAGGUGGCCGUGGAGGCCU |
| | | | GCAACGCUAUGCUGAAGGAGAACUUCCCCACCGUGGCCAGCUACUG |
| | | | CAUCAUCCCCGAGUACGACGCCUACCUGGACAUGGUGGACGGCGCC |
| | | | AGCUGCUGCCUGGACACCGCCAGCUUCUGCCCCGCCAAGCUGAGGA |
| | | | GCUUCCCCAAGAAACACAGCUACCUGGAGCCCACCAUCAGGAGCGC |
| | | | CGUGCCCAGCGCCAUCCAGAACACCCUGCAGAACGUGCUGGCCGCU |
| | | | GCCACCAAGAGGAACUGCAACGUGACCCAGAUGAGGGAGCUGCCCG |
| | | | UGCUGGACAGCGCUGCCUUCAACGUGGAGUGCUUCAAGAAAUACGC |
| | | | CUGCAACAACGAGUACUGGGAGACCUUCAAGGAGAACCCCAUCAGG |
| | | | CUGACCGAAGAGAACGUGGUGAACUACAUCACCAAGCUGAAGGGCC |
| | | | CCAAGGCCGCUGCCCUGUUCGCUAAGACCCACAACCUGAACAUGCU |
| | | | GCAGGACAUCCCAAUGGACAGGUUCGUGAUGGACCUGAAGAGGGAC |
| | | | GUGAAGGUGACACCCGGCACCAAGCACACCGAGGAGAGGCCCAAGG |
| | | | UGCAGGUGAUCCAGGCCGCUGACCCACUGGCCACCGCCUACCUGUG |
| | | | CGGCAUCCACAGGGAGCUGGUGAGGCGGCUGAACGCCGUGCUGCUG |
| | | | CCCAACAUCCACACCCUGUUCGACAUGAGCGCCGAGGACUUCGACG |
| | | | CCAUCAUCGCCGAGCACUUCCAGCCCGGCGACUGCGUGCUGGAGAC |
| | | | CGACAUCGCCAGCUUCGACAAGAGCGAGGAUGACGCUAUGGCCCUG |
| | | | ACCGCUCUGAUGAUCCUGGAGGACCUGGGCGUGGACGCCGAGCUGC |
| | | | UCACCCUGAUCGAGGCUGCCUUCGGCGAGAUCAGCUCCAUCCACCU |
| | | | GCCCACCAAGACCAAGUUCAAGUUCGGCGCUAUGAUGAAAAGCGGA |
| | | | AUGUUCCUGACCCUGUUCGUGAACACCGUGAUCAACAUUGUGAUCG |
| | | | CCAGCAGGGUGCUGCGGGAGAGGCUGACCGGCAGCCCCUGCGCUGC |
| | | | CUUCAUCGGCGACGACAACAUCGUGAAGGGCGUGAAAAGCGACAAG |
| | | | CUGAUGGCCGACAGGUGCGCCACCUGGCUGAACAUGGAGGUGAAGA |
| | | | UCAUCGACGCCGUGGUGGGCGAGAAGGCCCCCUACUUCUGCGGCGG |
| | | | AUUCAUCCUGUGCGACAGCGUGACCGGCACCGCCUGCAGGGUGGCC |
| | | | GACCCCCUGAAGAGGCUGUUCAAGCUGGGCAAGCCACUGGCCGCUG |
| | | | ACGAUGAGCACGACGAUGACAGGCGGAGGGCCCUGCACGAGGAAAG |
| | | | CACCAGGUGGAACAGGGUGGGCAUCCUGAGCGAGCUGUGCAAGGCC |
| | | | GUGGAGAGCAGGUACGAGACCGUGGGCACCAGCAUCAUCGUGAUGG |
| | | | CUAUGACCACACUGGCCAGCUCCGUCAAGAGCUUCUCCUACCUGAG |
| | | | GGGGGCCCCUAUAACUCUCUACGGCUAACCUGAAUGGACUACGACA |
| | | | UAGUCUAGUCCGCCAAGGCCGCCACCAUGAGAGUGACAGCCCCUAG |
| | | | AACCUUACUGCUUCUGCUUUGGGGAGCUGUUGCUCUGACAGAGACA |
| | | | UGGGCUGGAUCUCUGAGCGAGGUGACCGGCCAGGGCCUGUGCAUCG |
| | | | GCGCCGUGCCCAAGACCCACCAGGUGCUGUGCAACACCACCCAGAA |
| | | | GACCAGCGACGGCAGCUACUACCUGGCCGCUCCCACCGGCACCACC |
| | | | UGGGCCUGCAGCACCGGCCUGACCCCUUGCAUCAGCACCACCAUCC |
| | | | UGAACCUGACCACCGACUACUGCGUGCUGGUGGAGCUGUGGCCCAG |
| | | | GGUGACCUACCACAGCCCCAGCUACGCCUACCACCAGUUCGAGAGG |
| | | | AGGGCCAAGUACAAGAGGGAGCCCGUGAGCCUGACCCUGGCCCUGC |
| | | | UGCUGGGCGGCCUGACAAUGGGCGGCAUCGCCGCCGGCGUGGGCAC |
| | | | CGGCACCACCGCCCUGGUGGCCACCCAGCAGUUCCAGCAGCUGCAG |
| | | | GCCGCCAUGCACGACGACCUGAAGGAGGUGGAGAAGUCCAUCACCA |
| | | | ACCUGGAGAAGUCCCUGACCAGCCUGAGCGAGGUGGUGCUGCAGAA |
| | | | CAGGAGGGGCCUGGACCUGCUGUUCCUGAAGGAGGGCGGCCUGUGC |
| | | | GCCGCCCUGAAGGAGGAGUGCUGCCUGUACGCCGACCACACCGGCC |
| | | | UGGUGAUCGUGGGCAUUGUCGCUGGCCUGGCCGUCCUCGCCGUGGU |
| | | | GGUGAUUGGAGCUGUGGUCGCAGCUGUUAUGUGCAGAAGAAAGUCA |
| | | | UCCGGCGGAAAGGGAGGCUCCUACUCUCAGGCUGCUUCUGCUACAG |
| | | | UGCCUAGAGCUCUUAUGUGUUUAUCUCAGCUGUAAACUCGAGUAUG |
| | | | UUACGUGCAAAGGUGAUUGUCACCCCCCGAAAGACCAUAUUGUGAC |
| | | | ACACCCUCAGUAUCACGCCCAAACAUUUACAGCCGCGGUGUCAAAA |
| | | | ACCGCGUGGACGUGGUUAACAUCCCUGCUGGGAGGAUCAGCCGUAA |
| | | | UUAUUAUAAUUGGCUUGGUGCUGGCUACUAUUGUGGCCAUGUACGU |
| | | | GCUGACCAACCAGAAACAUAAUUGAAUACAGCAGCAAUUGGCAAGC |
| | | | UGCUUACAUAGAACUCGCGGCGAUUGGCAUGCCGCCUUAAAAUUUU |
| | | | UAUUUUAUUUUUUCUUUUCUUUUCCGAAUCGGAUUUUGUUUUUAAU |
| | | | AUUUCAAAAAAAAAAAAAAAAAAAAAAAAAUCUAGAAAAAAAAAAA |
| | | | AAAAAAAAAAAAAAAAAAAAAAAAAAAAAAAAAAAAAAAAAAAAAA |
| | | | AAAAAAAAAAAAAAAAAAAAAAAAAAAAAAAAAAAAAAAAAAA |
| 3061 | STARR^{TM} | 3061 | AUGGGCGGCGCAUGAGAGAAGCCCAGACCAAUUACCUACCCAAAAU |
| (SEQ | AH1A5 | | GGAGAAAGUUCACGUUGACAUCGAGGAAGACAGCCCAUUCCUCAGA |
| ID | | | GCUUUGCAGCGGAGCUUCCCGCAGUUUGAGGUAGAAGCCAAGCAGG |
| NO: 106) | | | UCACUGAUAAUGACCAUGCUAAUGCCAGAGCGUUUUCGCAUCUGGC |
| | | | UUCAAAACUGAUCGAAACGGAGGUGGACCCAUCCGACACGAUCCUU |
| | | | GACAUUGGAAGUGCGCCCGCCCGCAGAAUGUAUUCUAAGCACAAGU |
| | | | AUCAUUGUAUCUGUCCGAUGAGAUGUGCGGAAGAUCCGGACAGAUU |
| | | | GUAUAAGUAUGCAACUAAGCUGAAGAAAAACUGUAAGGAAAUAACU |
| | | | GAUAAGGAAUUGGACAAGAAAAUGAAGGAGCUGGCCGCCGUCAUGA |
| | | | GCGACCCUGACCUGGAAACUGAGACUAUGUGCCUCCACGACGACGA |
| | | | GUCGUGUCGCUACGAAGGGCAAGUCGCUGUUUACCAGGAUGUAUAC |
| | | | GCCGUCGACGGCCCCACCAGCCUGUACCACCAGGCCAACAAGGGCG |
| | | | UGAGGGUGGCCUACUGGAUCGGCUUCGACACCACACCCUUCAUGUU |
| | | | CAAGAACCUGGCCGGCGCCUACCCCAGCUACAGCACCAACUGGGCC |
| | | | GACGAGACCGUGCUGACCGCCAGGAACAUCGGCCUGUGCAGCAGCG |
| | | | ACGYGAYGGAGAGGAGCCGGAGAGGCAYGAGCAYCCYGAGGAAGAA |
| | | | AUACCUGAAGCCCAGCAACAACGUGCUGUUCAGCGUGGGCAGCACC |
| | | | AUCUACCACGAGAAGAGGGACCUGCUCAGGAGCUGGCACCUGCCCA |
| | | | GCGUGUUCCACCUGAGGGGCAAGCAGAACUACACCUGCAGGUGCGA |
| | | | GACCAUCGUGAGCUGCGACGGCUACGUGGUGAAGAGGAUCGCCAUC |
| | | | AGCCCCGGCCUGUACGGCAAGCCCAGCGGCUACGCCGCUACAAUGC |
| | | | ACAGGGAGGGCUUCCUGUGCUGCAAGGUGACCGACACCCUGAACGG |
| | | | CGAGAGGGUGAGCUUCCCCGUGUGCACCUACGUGCCCGCCACCCUG |
| | | | UGCGACCAGAUGACCGGCAUCCUGGCCACCGACGUGAGCGCCGACG |
| | | | ACGCCCAGAAGCUGCUCGUGGGCCUGAACCAGAGGAUCGUGGUCAA |
| | | | CGGCAGGACCCAGAGGAACACCAACACAAUGAAGAACUACCUGCUG |
| | | | CCCGUGGUGGCCCAGGCUUUCGCCAGGUGGGCCAAGGAGUACAAGG |
| | | | AGGACCAGGAAGACGAGAGGCCCCUGGGCCUGAGGGACAGGCAGCU |
| | | | GGUGAUGGGCUGCUGCUGGGCCUUCAGGCGGCACAAGAUCACCAGC |
| | | | AUCUACAAGAGGCCCGACACCCAGACCAUCAUCAAGGUGAACAGCG |
| | | | ACUUCCACAGCUUCGUGCUGCCCAGGAUCGGCAGCAACACCCUGGA |
| | | | GAUCGGCCUGAGGACCCGGAUCAGGAAGAUGCUGGAGGAACACAAG |
| | | | GAGCCCAGCCCACUGAUCACCGCCGAGGACGUGCAGGAGGCCAAGU |
| | | | GCGCUGCCGACGAGGCCAAGGAGGUGAGGGAGGCCGAGGAACUGAG |
| | | | GGCCGCCCUGCCACCCCUGGCUGCCGACGUGGAGGAACCCACCCUG |
| | | | GAAGCCGACGUGGACCUGAUGCUGCAGGAGGCCGGCGCCGGAAGCG |
| | | | UGGAGACACCCAGGGGCCUGAUCAAGGUGACCAGCUACGACGGCGA |
| | | | GGACAAGAUCGGCAGCUACGCCGUGCUGAGCCCACAGGCCGUGCUG |
| | | | AAGUCCGAGAAGCUGAGCUGCAUCCACCCACUGGCCGAGCAGGUGA |
| | | | UCGUGAUCACCCACAGCGGCAGGAAGGGCAGGUACGCCGUGGAGCC |
| | | | CUACCACGGCAAGGUGGUCGUGCCCGAGGGCCACGCCAUCCCCGUG |
| | | | CAGGACUUCCAGGCCCUGAGCGAGAGCGCCACCAUCGUGUACAACG |
| | | | AGAGGGAGUUCGUGAACAGGUACCUGCACCAUAUCGCCACCCACGG |
| | | | CGGAGCCCUGAACACCGACGAGGAAUACUACAAGACCGUGAAGCCC |
| | | | AGCGAGCACGACGGCGAGUACCUGUACGACAUCGACAGGAAGCAGU |
| | | | GCGUGAAGAAAGAGCUGGUGACCGGCCUGGGACUGACCGGCGAGCU |
| | | | GGUGGACCCACCCUUCCACGAGUUCGCCUACGAGAGCCUGAGGACC |
| | | | AGACCCGCCGCUCCCUACCAGGUGCCCACCAUCGGCGUGUACGGCG |
| | | | UGCCCGGCAGCGGAAAGAGCGGCAUCAUCAAGAGCGCCGUGACCAA |
| | | | GAAAGACCUGGUGGUCAGCGCCAAGAAAGAGAACUGCGCCGAGAUC |
| | | | AUCAGGGACGUGAAGAAGAUGAAAGGCCUGGACGUGAACGCGCGCA |
| | | | CCGUGGACAGCGUGCUGCUGAACGGCUGCAAGCACCCCGUGGAGAC |
| | | | CCUGUACAUCGACGAGGCCUUCGCUUGCCACGCCGGCACCCUGAGG |
| | | | GCCCUGAUCGCCAUCAUCAGGCCCAAGAAAGCCGUGCUGUGCGGCG |
| | | | ACCCCAAGCAGUGCGGCUUCUUCAACAUGAUGUGCCUGAAGGUGCA |
| | | | CUUCAACCACGAGAUCUGCACCCAGGUGUUCCACAAGAGCAUCAGC |
| | | | AGGCGGUGCACCAAGAGCGUGACCAGCGUCGUGAGCACCCUGUUCU |
| | | | ACGACAAGAAAAUGAGGACCACCAACCCCAAGGAGACCAAAAUCGU |
| | | | GAUCGACACCACAGGCAGCACCAAGCCCAAGCAGGACGACCUGAUC |
| | | | CUGACCUGCUUCAGGGGCUGGGUGAAGCAGCUGCAGAUCGACUACA |
| | | | AGGGCAACGAGAUCAUGACCGCCGCUGCCAGCCAGGGCCUGACCAG |
| | | | GAAGGGCGUGUACGCCGUGAGGUACAAGGUGAACGAGAACCCACUG |
| | | | UACGCUCCCACCAGCGAGCACGUGAACGUGCUGCUGACCAGGACCG |
| | | | AGGACAGGAUCGUGUGGAAGACCCUGGCCGGCGACCCCUGGAUCAA |
| | | | GACCCUGACCGCCAAGUACCCCGGCAACUUCACCGCCACCAUCGAA |
| | | | GAGUGGCAGGCCGAGCACGACGCCAUCAUGAGGCACAUCCUGGAGA |
| | | | GGCCCGACCCCACCGACGUGUUCCAGAACAAGGCCAACGUGUGCUG |
| | | | GGCCAAGGCCCUGGUGCCCGUGCUGAAGACCGCCGGCAUCGACAUG |
| | | | ACCACAGAGCAGUGGAACACCGUGGACUACUUCGAGACCGACAAGG |
| | | | CCCACAGCGCCGAGAUCGUGCUGAACCAGCUGUGCGUGAGGUUCUU |
| | | | CGGCCUGGACCUGGACAGCGGCCUGUUCAGCGCCCCCACCGUGCCA |
| | | | CUGAGCAUCAGGAACAACCACUGGGACAACAGCCCCAGCCCAAACA |
| | | | UGUACGGCCUGAACAAGGAGGUGGUCAGGCAGCUGAGCAGGCGGUA |
| | | | CCCACAGCUGCCCAGGGCCGUGGCCACCGGCAGGGUGUACGACAUG |
| | | | AACACCGGCACCCUGAGGAACUACGACCCCAGGAUCAACCUGGUGC |
| | | | CCGUGAACAGGCGGCUGCCCCACGCCCUGGUGCUGCACCACAACGA |
| | | | GCACCCACAGAGCGACUUCAGCUCCUUCGUGAGCAAGCUGAAAGGC |
| | | | AGGACCGUGCUGGUCGUGGGCGAGAAGCUGAGCGUGCCCGGCAAGA |
| | | | UGGUGGACUGGCUGAGCGACAGGCCCGAGGCCACCUUCCGGGCCAG |
| | | | GCUGGACCUCGGCAUCCCCGGCGACGUGCCCAAGUACGACAUCAUC |
| | | | UUCGUGAACGUCAGGACCCCAUACAAGUACCACCAUUACCAGCAGU |
| | | | GCGAGGACCACGCCAUCAAGCUGAGCAUGCUGACCAAGAAGGCCUG |
| | | | CCUGCACCUGAACCCCGGAGGCACCUGCGUGAGCAUCGGCUACGGC |
| | | | UACGCCGACAGGGCCAGCGAGAGCAUCAUUGGCGCCAUCGCCAGGC |
| | | | UGUUCAAGUUCAGCAGGGUGUGCAAACCCAAGAGCAGCCUGGAGGA |
| | | | AACCGAGGUGCUGUUCGUGUUCAUCGGCUACGACCGGAAGGCCAGG |
| | | | ACCCACAACCCCUACAAGCUGAGCAGCACCCUGACAAACAUCUACA |
| | | | CCGGCAGCAGGCUGCACGAGGCCGGCUGCGCCCCCAGCUACCACGU |
| | | | GGUCAGGGGCGAUAUCGCCACCGCCACCGAGGGCGUGAUCAUCAAC |
| | | | GCUGCCAACAGCAAGGGCCAGCCCGGAGGCGGAGUGUGCGGCGCCC |
| | | | UGUACAAGAAGUUCCCCGAGAGCUUCGACCUGCAGCCCAUCGAGGU |
| | | | GGGCAAGGCCAGGCUGGUGAAGGGCGCCGCUAAGCACAUCAUCCAC |
| | | | GCCGUGGGCCCCAACUUCAACAAGGUGAGCGAGGUGGAAGGCGACA |
| | | | AGCAGCUGGCCGAAGCCUACGAGAGCAUCGCCAAGAUCGUGAACGA |
| | | | CAAUAACUACAAGAGCGUGGCCAUCCCACUGCUCAGCACCGGCAUC |
| | | | UUCAGCGGCAACAAGGACAGGCUGACCCAGAGCCUGAACCACCUGC |
| | | | UCACCGCCCUGGACACCACCGAUGCCGACGUGGCCAUCUACUGCAG |
| | | | GGACAAGAAGUGGGAGAUGACCCUGAAGGAGGCCGUGGCCAGGCGG |
| | | | GAGGCCGUGGAAGAGAUCUGCAUCAGCGACGACUCCAGCGUGACCG |
| | | | AGCCCGACGCCGAGCUGGUGAGGGUGCACCCCAAGAGCUCCCUGGC |
| | | | CGGCAGGAAGGGCUACAGCACCAGCGACGGCAAGACCUUCAGCUAC |
| | | | CUGGAGGGCACCAAGUUCCACCAGGCCGCUAAGGACAUCGCCGAGA |
| | | | UCAACGCUAUGUGGCCCGUGGCCACCGAGGCCAACGAGCAGGUGUG |
| | | | CAUGUACAUCCUGGGCGAGAGCAUGUCCAGCAUCAGGAGCAAGUGC |
| | | | CCCGUGGAGGAAAGCGAGGCCAGCACACCACCCAGCACCCUGCCCU |
| | | | GCCUGUGCAUCCACGCUAUGACACCCGAGAGGGUGCAGCGGCUGAA |
| | | | GGCCAGCAGGCCCGAGCAGAUCACCGUGUGCAGCUCCUUCCCACUG |
| | | | CCCAAGUACAGGAUCACCGGCGUGCAGAAGAUCCAGUGCAGCCAGC |
| | | | CCAUCCUGUUCAGCCCAAAGGUGCCCGCCUACAUCCACCCCAGGAA |
| | | | GUACCUGGUGGAGACCCCACCCGUGGACGAGACACCCGAGCCAAGC |
| | | | GCCGAGAACCAGAGCACCGAGGGCACACCCGAGCAGCCACCCCUGA |
| | | | UCACCGAGGACGAGACAAGGACCCGGACCCCAGAGCCCAUCAUUAU |
| | | | CGAGGAAGAGGAAGAGGACAGCAUCAGCCUGCUGAGCGACGGCCCC |
| | | | ACCCACCAGGUGCUGCAGGUGGAGGCCGACAUCCACGGCCCACCCA |
| | | | GCGUGUCCAGCUCCAGCUGGAGCAUCCCACACGCCAGCGACUUCGA |
| | | | CGUGGACAGCCUGAGCAUCCUGGACACCCUGGAGGGCGCCAGCGUG |
| | | | ACCUCCGGCGCCACCAGCGCCGAGACCAACAGCUACUUCGCCAAGA |
| | | | GCAUGGAGUUCCUGGCCAGGCCCGUGCCAGCUCCCAGGACCGUGUU |
| | | | CAGGAACCCACCCCACCCAGCUCCCAGGACCAGGACCCCAAGCCUG |
| | | | GCUCCCAGCAGGGCCUGCAGCAGGACCAGCCUGGUGAGCACCCCAC |
| | | | CCGGCGUGAACAGGGUGAUCACCAGGGAGGAACUGGAGGCCCUGAC |
| | | | ACCCAGCAGGACCCCCAGCAGGUCCGUGAGCAGGACUAGUCUGGUG |
| | | | UCCAACCCACCCGGCGUGAACAGGGUGAUCACCAGGGAGGAAUUCG |
| | | | AGGCCUUCGUGGCCCAGCAACAGAGACGGUUCGACGCCGGCGCCUA |
| | | | CAUCUUCAGCAGCGACACCGGCCAGGGACACCUGCAGCAAAAGAGC |
| | | | GUGAGGCAGACCGUGCUGAGCGAGGUGGUGCUGGAGAGGACCGAGC |
| | | | UGGAAAUCAGCUACGCCCCCAGGCUGGACCAGGAGAAGGAGGAACU |
| | | | GCUCAGGAAGAAACUGCAGCUGAACCCCACCCCAGCCAACAGGAGC |
| | | | AGGUACCAGAGCAGGAAGGUGGAGAACAUGAAGGCCAUCACCGCCA |
| | | | GGCGGAUCCUGCAGGGCCUGGGACACUACCUGAAGGCCGAGGGCAA |
| | | | GGUGGAGUGCUACAGGACCCUGCACCCCGUGCCACUGUACAGCUCC |
| | | | AGCGUGAACAGGGCCUUCUCCAGCCCCAAGGUGGCCGUGGAGGCCU |
| | | | GCAACGCUAUGCUGAAGGAGAACUUCCCCACCGUGGCCAGCUACUG |
| | | | CAUCAUCCCCGAGUACGACGCCUACCUGGACAUGGUGGACGGCGCC |
| | | | AGCUGCUGCCUGGACACCGCCAGCUUCUGCCCCGCCAAGCUGAGGA |
| | | | GCUUCCCCAAGAAACACAGCUACCUGGAGCCCACCAUCAGGAGCGC |
| | | | CGUGCCCAGCGCCAUCCAGAACACCCUGCAGAACGUGCUGGCCGCU |
| | | | GCCACCAAGAGGAACUGCAACGUGACCCAGAUGAGGGAGCUGCCCG |
| | | | UGCUGGACAGCGCUGCCUUCAACGUGGAGUGCUUCAAGAAAUACGC |
| | | | CUGACCGAAGAGAACGUGGUGAACUACAUCACCAAGCUGAAGGGCC |
| | | | CCAAGGCCGCUGCCCUGUUCGCUAAGACCCACAACCUGAACAUGCU |
| | | | GCAGGACAUCCCAAUGGACAGGUUCGUGAUGGACCUGAAGAGGGAC |
| | | | GUGAAGGUGACACCCGGCACCAAGCACACCGAGGAGAGGCCCAAGG |
| | | | UGCAGGUGAUCCAGGCCGCUGACCCACUGGCCACCGCCUACCUGUG |
| | | | CGGCAUCCACAGGGAGCUGGUGAGGCGGCUGAACGCCGUGCUGCUG |
| | | | CCCAACAUCCACACCCUGUUCGACAUGAGCGCCGAGGACUUCGACG |
| | | | CCAUCAUCGCCGAGCACUUCCAGCCCGGCGACUGCGUGCUGGAGAC |
| | | | CGACAUCGCCAGCUUCGACAAGAGCGAGGAUGACGCUAUGGCCCUG |
| | | | ACCGCUCUGAUGAUCCUGGAGGACCUGGGCGUGGACGCCGAGCUGC |
| | | | UCACCCUGAUCGAGGCUGCCUUCGGCGAGAUCAGCUCCAUCCACCU |
| | | | GCCCACCAAGACCAAGUUCAAGUUCGGCGCUAUGAUGAAAAGCGGA |
| | | | AUGUUCCUGACCCUGUUCGUGAACACCGUGAUCAACAUUGUGAUCG |
| | | | CCAGCAGGGUGCUGCGGGAGAGGCUGACCGGCAGCCCCUGCGCUGC |
| | | | CUUCAUCGGCGACGACAACAUCGUGAAGGGCGUGAAAAGCGACAAG |
| | | | CUGAUGGCCGACAGGUGCGCCACCUGGCUGAACAUGGAGGUGAAGA |
| | | | UCAUCGACGCCGUGGUGGGCGAGAAGGCCCCCUACUUCUGCGGCGG |
| | | | AUUCAUCCUGUGCGACAGCGUGACCGGCACCGCCUGCAGGGUGGCC |
| | | | GACCCCCUGAAGAGGCUGUUCAAGCUGGGCAAGCCACUGGCCGCUG |
| | | | ACGAUGAGCACGACGAUGACAGGCGGAGGGCCCUGCACGAGGAAAG |
| | | | CACCAGGUGGAACAGGGUGGGCAUCCUGAGCGAGCUGUGCAAGGCC |
| | | | GUGGAGAGCAGGUACGAGACCGUGGGCACCAGCAUCAUCGUGAUGG |
| | | | CUAUGACCACACUGGCCAGCUCCGUCAAGAGCUUCUCCUACCUGAG |
| | | | GGGGGCCCCUAUAACUCUCUACGGCUAACCUGAAUGGACUACGACA |
| | | | UAGUCUAGUCCGCCAAGGCCGCCACCAUGAGAGUGACAGCCCCUAG |
| | | | AACCUUACUGCUUCUGCUUUGGGGAGCUGUUGCUCUGACAGAGACA |
| | | | UGGGCUGGAUCUUACCACAGCCCCAGCUACGCCUACCACCAGUUCG |
| | | | AGAGGGGGGGAGGAGGCUCCGGGGGAGGAGGCUCCCUGAAGAUCAG |
| | | | CCAGGCCGUGCACGCCGCCCACGCCGAGAUCAACGAGGCCGGCCGG |
| | | | GAGGUGAUCGUGGGCAUUGUCGCUGGCCUGGCCGUCCUCGCCGUGG |
| | | | UGGUGAUUGGAGCUGUGGUCGCAGCUGUUAUGUGCAGAAGAAAGUC |
| | | | AUCCGGCGGAAAGGGAGGCUCCUACUCUCAGGCUGCUUCUGCUACA |
| | | | GUGCCUAGAGCUCUUAUGUGUUUAUCUCAGCUGUAAACUCGAGUAU |
| | | | GUUACGUGCAAAGGUGAUUGUCACCCCCCGAAAGACCAUAUUGUGA |
| | | | CACACCCUCAGUAUCACGCCCAAACAUUUACAGCCGCGGUGUCAAA |
| | | | AACCGCGUGGACGUGGUUAACAUCCCUGCUGGGAGGAUCAGCCGUA |
| | | | AUUAUUAUAAUUGGCUUGGUGCUGGCUACUAUUGUGGCCAUGUACG |
| | | | UGCUGACCAACCAGAAACAUAAUUGAAUACAGCAGCAAUUGGCAAG |
| | | | CUGCUUACAUAGAACUCGCGGCGAUUGGCAUGCCGCCUUAAAAUUU |
| | | | UUAUUUUAUUUUUUCUUUUCUUUUCCGAAUCGGAUUUUGUUUUUAA |
| | | | UAUUUCAAAAAAAAAAAAAAAAAAAAAAAAAUCUAGAAAAAAAAAA |
| | | | AAAAAAAAAAAAAAAAAAAAAAAAAAAAAAAAAAAAAAAAAAAAAA |
| | | | AAAAAAAAAAAAAAAAAAAAAAAAAAAAAAAAAAAAAAAAAAAA |
| 3067 | STARR^{TM} | 3067 | AUGGGCGGCGCAUGAGAGAAGCCCAGACCAAUUACCUACCCAAAAU |
| (SEQ | gp70- | | GGAGAAAGUUCACGUUGACAUCGAGGAAGACAGCCCAUUCCUCAGA |
| ID | FLAG | | GCUUUGCAGCGGAGCUUCCCGCAGUUUGAGGUAGAAGCCAAGCAGG |
| NO: 107) | | | UCACUGAUAAUGACCAUGCUAAUGCCAGAGCGUUUUCGCAUCUGGC |
| | | | UUCAAAACUGAUCGAAACGGAGGUGGACCCAUCCGACACGAUCCUU |
| | | | GACAUUGGAAGUGCGCCCGCCCGCAGAAUGUAUUCUAAGCACAAGU |
| | | | AUCAUUGUAUCUGUCCGAUGAGAUGUGCGGAAGAUCCGGACAGAUU |
| | | | GUAUAAGUAUGCAACUAAGCUGAAGAAAAACUGUAAGGAAAUAACU |
| | | | GAUAAGGAAUUGGACAAGAAAAUGAAGGAGCUGGCCGCCGUCAUGA |
| | | | GCGACCCUGACCUGGAAACUGAGACUAUGUGCCUCCACGACGACGA |
| | | | GUCGUGUCGCUACGAAGGGCAAGUCGCUGUUUACCAGGAUGUAUAC |
| | | | GCCGUCGACGGCCCCACCAGCCUGUACCACCAGGCCAACAAGGGCG |
| | | | UGAGGGUGGCCUACUGGAUCGGCUUCGACACCACACCCUUCAUGUU |
| | | | CAAGAACCUGGCCGGCGCCUACCCCAGCUACAGCACCAACUGGGCC |
| | | | GACGAGACCGUGCUGACCGCCAGGAACAUCGGCCUGUGCAGCAGCG |
| | | | ACGUGAUGGAGAGGAGCCGGAGAGGCAUGAGCAUCCUGAGGAAGAA |
| | | | AUACCUGAAGCCCAGCAACAACGUGCUGUUCAGCGUGGGCAGCACC |
| | | | AUCUACCACGAGAAGAGGGACCUGCUCAGGAGCUGGCACCUGCCCA |
| | | | GCGUGUUCCACCUGAGGGGCAAGCAGAACUACACCUGCAGGUGCGA |
| | | | GACCAUCGUGAGCUGCGACGGCUACGUGGUGAAGAGGAUCGCCAUC |
| | | | AGCCCCGGCCUGUACGGCAAGCCCAGCGGCUACGCCGCUACAAUGC |
| | | | ACAGGGAGGGCUUCCUGUGCUGCAAGGUGACCGACACCCUGAACGG |
| | | | CGAGAGGGUGAGCUUCCCCGUGUGCACCUACGUGCCCGCCACCCUG |
| | | | UGCGACCAGAUGACCGGCAUCCUGGCCACCGACGUGAGCGCCGACG |
| | | | ACGCCCAGAAGCUGCUCGUGGGCCUGAACCAGAGGAUCGUGGUCAA |
| | | | CGGCAGGACCCAGAGGAACACCAACACAAUGAAGAACUACCUGCUG |
| | | | CCCGUGGUGGCCCAGGCUUUCGCCAGGUGGGCCAAGGAGUACAAGG |
| | | | AGGACCAGGAAGACGAGAGGCCCCUGGGCCUGAGGGACAGGCAGCU |
| | | | GGUGAUGGGCUGCUGCUGGGCCUUCAGGCGGCACAAGAUCACCAGC |
| | | | AUCUACAAGAGGCCCGACACCCAGACCAUCAUCAAGGUGAACAGCG |
| | | | ACUUCCACAGCUUCGUGCUGCCCAGGAUCGGCAGCAACACCCUGGA |
| | | | GAUCGGCCUGAGGACCCGGAUCAGGAAGAUGCUGGAGGAACACAAG |
| | | | GAGCCCAGCCCACUGAUCACCGCCGAGGACGUGCAGGAGGCCAAGU |
| | | | GCGCUGCCGACGAGGCCAAGGAGGUGAGGGAGGCCGAGGAACUGAG |
| | | | GGCCGCCCUGCCACCCCUGGCUGCCGACGUGGAGGAACCCACCCUG |
| | | | GAAGCCGACGUGGACCUGAUGCUGCAGGAGGCCGGCGCCGGAAGCG |
| | | | UGGAGACACCCAGGGGCCUGAUCAAGGUGACCAGCUACGACGGCGA |
| | | | GGACAAGAUCGGCAGCUACGCCGUGCUGAGCCCACAGGCCGUGCUG |
| | | | AAGUCCGAGAAGCUGAGCUGCAUCCACCCACUGGCCGAGCAGGUGA |
| | | | UCGUGAUCACCCACAGCGGCAGGAAGGGCAGGUACGCCGUGGAGCC |
| | | | CUACCACGGCAAGGUGGUCGUGCCCGAGGGCCACGCCAUCCCCGUG |
| | | | CAGGACUUCCAGGCCCUGAGCGAGAGCGCCACCAUCGUGUACAACG |
| | | | AGAGGGAGUUCGUGAACAGGUACCUGCACCAUAUCGCCACCCACGG |
| | | | CGGAGCCCUGAACACCGACGAGGAAUACUACAAGACCGUGAAGCCC |
| | | | AGCGAGCACGACGGCGAGUACCUGUACGACAUCGACAGGAAGCAGU |
| | | | GCGUGAAGAAAGAGCUGGUGACCGGCCUGGGACUGACCGGCGAGCU |
| | | | GGUGGACCCACCCUUCCACGAGUUCGCCUACGAGAGCCUGAGGACC |
| | | | AGACCCGCCGCUCCCUACCAGGUGCCCACCAUCGGCGUGUACGGCG |
| | | | UGCCCGGCAGCGGAAAGAGCGGCAUCAUCAAGAGCGCCGUGACCAA |
| | | | GAAAGACCUGGUGGUCAGCGCCAAGAAAGAGAACUGCGCCGAGAUC |
| | | | AUCAGGGACGUGAAGAAGAUGAAAGGCCUGGACGUGAACGCGCGCA |
| | | | CCGUGGACAGCGUGCUGCUGAACGGCUGCAAGCACCCCGUGGAGAC |
| | | | CCUGUACAUCGACGAGGCCUUCGCUUGCCACGCCGGCACCCUGAGG |
| | | | GCCCUGAUCGCCAUCAUCAGGCCCAAGAAAGCCGUGCUGUGCGGCG |
| | | | ACCCCAAGCAGUGCGGCUUCUUCAACAUGAUGUGCCUGAAGGUGCA |
| | | | CUUCAACCACGAGAUCUGCACCCAGGUGUUCCACAAGAGCAUCAGC |
| | | | AGGCGGUGCACCAAGAGCGUGACCAGCGUCGUGAGCACCCUGUUCU |
| | | | ACGACAAGAAAAUGAGGACCACCAACCCCAAGGAGACCAAAAUCGU |
| | | | GAUCGACACCACAGGCAGCACCAAGCCCAAGCAGGACGACCUGAUC |
| | | | CUGACCUGCUUCAGGGGCUGGGUGAAGCAGCUGCAGAUCGACUACA |
| | | | AGGGCAACGAGAUCAUGACCGCCGCUGCCAGCCAGGGCCUGACCAG |
| | | | GAAGGGCGUGUACGCCGUGAGGUACAAGGUGAACGAGAACCCACUG |
| | | | UACGCUCCCACCAGCGAGCACGUGAACGUGCUGCUGACCAGGACCG |
| | | | AGGACAGGAUCGUGUGGAAGACCCUGGCCGGCGACCCCUGGAUCAA |
| | | | GACCCUGACCGCCAAGUACCCCGGCAACUUCACCGCCACCAUCGAA |
| | | | GAGUGGCAGGCCGAGCACGACGCCAUCAUGAGGCACAUCCUGGAGA |
| | | | GGCCCGACCCCACCGACGUGUUCCAGAACAAGGCCAACGUGUGCUG |
| | | | GGCCAAGGCCCUGGUGCCCGUGCUGAAGACCGCCGGCAUCGACAUG |
| | | | ACCACAGAGCAGUGGAACACCGUGGACUACUUCGAGACCGACAAGG |
| | | | CCCACAGCGCCGAGAUCGUGCUGAACCAGCUGUGCGUGAGGUUCUU |
| | | | CGGCCUGGACCUGGACAGCGGCCUGUUCAGCGCCCCCACCGUGCCA |
| | | | CUGAGCAUCAGGAACAACCACUGGGACAACAGCCCCAGCCCAAACA |
| | | | UGUACGGCCUGAACAAGGAGGUGGUCAGGCAGCUGAGCAGGCGGUA |
| | | | CCCACAGCUGCCCAGGGCCGUGGCCACCGGCAGGGUGUACGACAUG |
| | | | AACACCGGCACCCUGAGGAACUACGACCCCAGGAUCAACCUGGUGC |
| | | | CCGUGAACAGGCGGCUGCCCCACGCCCUGGUGCUGCACCACAACGA |
| | | | GCACCCACAGAGCGACUUCAGCUCCUUCGUGAGCAAGCUGAAAGGC |
| | | | AGGACCGUGCUGGUCGUGGGCGAGAAGCUGAGCGUGCCCGGCAAGA |
| | | | UGGUGGACUGGCUGAGCGACAGGCCCGAGGCCACCUUCCGGGCCAG |
| | | | GCUGGACCUCGGCAUCCCCGGCGACGUGCCCAAGUACGACAUCAUC |
| | | | UUCGUGAACGUCAGGACCCCAUACAAGUACCACCAUUACCAGCAGU |
| | | | GCGAGGACCACGCCAUCAAGCUGAGCAUGCUGACCAAGAAGGCCUG |
| | | | CCUGCACCUGAACCCCGGAGGCACCUGCGUGAGCAUCGGCUACGGC |
| | | | UACGCCGACAGGGCCAGCGAGAGCAUCAUUGGCGCCAUCGCCAGGC |
| | | | UGUUCAAGUUCAGCAGGGUGUGCAAACCCAAGAGCAGCCUGGAGGA |
| | | | AACCGAGGUGCUGUUCGUGUUCAUCGGCUACGACCGGAAGGCCAGG |
| | | | ACCCACAACCCCUACAAGCUGAGCAGCACCCUGACAAACAUCUACA |
| | | | CCGGCAGCAGGCUGCACGAGGCCGGCUGCGCCCCCAGCUACCACGU |
| | | | GGUCAGGGGCGAUAUCGCCACCGCCACCGAGGGCGUGAUCAUCAAC |
| | | | GCUGCCAACAGCAAGGGCCAGCCCGGAGGCGGAGUGUGCGGCGCCC |
| | | | UGUACAAGAAGUUCCCCGAGAGCUUCGACCUGCAGCCCAUCGAGGU |
| | | | GGGCAAGGCCAGGCUGGUGAAGGGCGCCGCUAAGCACAUCAUCCAC |
| | | | GCCGUGGGCCCCAACUUCAACAAGGUGAGCGAGGUGGAAGGCGACA |
| | | | AGCAGCUGGCCGAAGCCUACGAGAGCAUCGCCAAGAUCGUGAACGA |
| | | | CAAUAACUACAAGAGCGUGGCCAUCCCACUGCUCAGCACCGGCAUC |
| | | | UUCAGCGGCAACAAGGACAGGCUGACCCAGAGCCUGAACCACCUGC |
| | | | UCACCGCCCUGGACACCACCGAUGCCGACGUGGCCAUCUACUGCAG |
| | | | GGACAAGAAGUGGGAGAUGACCCUGAAGGAGGCCGUGGCCAGGCGG |
| | | | GAGGCCGUGGAAGAGAUCUGCAUCAGCGACGACUCCAGCGUGACCG |
| | | | AGCCCGACGCCGAGCUGGUGAGGGUGCACCCCAAGAGCUCCCUGGC |
| | | | CGGCAGGAAGGGCUACAGCACCAGCGACGGCAAGACCUUCAGCUAC |
| | | | CUGGAGGGCACCAAGUUCCACCAGGCCGCUAAGGACAUCGCCGAGA |
| | | | UCAACGCUAUGUGGCCCGUGGCCACCGAGGCCAACGAGCAGGUGUG |
| | | | CAUGUACAUCCUGGGCGAGAGCAUGUCCAGCAUCAGGAGCAAGUGC |
| | | | CCCGUGGAGGAAAGCGAGGCCAGCACACCACCCAGCACCCUGCCCU |
| | | | GCCUGUGCAUCCACGCUAUGACACCCGAGAGGGUGCAGCGGCUGAA |
| | | | GGCCAGCAGGCCCGAGCAGAUCACCGUGUGCAGCUCCUUCCCACUG |
| | | | CCCAAGUACAGGAUCACCGGCGUGCAGAAGAUCCAGUGCAGCCAGC |
| | | | CCAUCCUGUUCAGCCCAAAGGUGCCCGCCUACAUCCACCCCAGGAA |
| | | | GUACCUGGUGGAGACCCCACCCGUGGACGAGACACCCGAGCCAAGC |
| | | | GCCGAGAACCAGAGCACCGAGGGCACACCCGAGCAGCCACCCCUGA |
| | | | UCACCGAGGACGAGACAAGGACCCGGACCCCAGAGCCCAUCAUUAU |
| | | | CGAGGAAGAGGAAGAGGACAGCAUCAGCCUGCUGAGCGACGGCCCC |
| | | | ACCCACCAGGUGCUGCAGGUGGAGGCCGACAUCCACGGCCCACCCA |
| | | | GCGUGUCCAGCUCCAGCUGGAGCAUCCCACACGCCAGCGACUUCGA |
| | | | CGUGGACAGCCUGAGCAUCCUGGACACCCUGGAGGGCGCCAGCGUG |
| | | | ACCUCCGGCGCCACCAGCGCCGAGACCAACAGCUACUUCGCCAAGA |
| | | | GCAUGGAGUUCCUGGCCAGGCCCGUGCCAGCUCCCAGGACCGUGUU |
| | | | CAGGAACCCACCCCACCCAGCUCCCAGGACCAGGACCCCAAGCCUG |
| | | | GCUCCCAGCAGGGCCUGCAGCAGGACCAGCCUGGUGAGCACCCCAC |
| | | | CCGGCGUGAACAGGGUGAUCACCAGGGAGGAACUGGAGGCCCUGAC |
| | | | ACCCAGCAGGACCCCCAGCAGGUCCGUGAGCAGGACUAGUCUGGUG |
| | | | UCCAACCCACCCGGCGUGAACAGGGUGAUCACCAGGGAGGAAUUCG |
| | | | AGGCCUUCGUGGCCCAGCAACAGAGACGGUUCGACGCCGGCGCCUA |
| | | | CAUCUUCAGCAGCGACACCGGCCAGGGACACCUGCAGCAAAAGAGC |
| | | | GUGAGGCAGACCGUGCUGAGCGAGGUGGUGCUGGAGAGGACCGAGC |
| | | | UGGAAAUCAGCUACGCCCCCAGGCUGGACCAGGAGAAGGAGGAACU |
| | | | GCUCAGGAAGAAACUGCAGCUGAACCCCACCCCAGCCAACAGGAGC |
| | | | AGGUACCAGAGCAGGAAGGUGGAGAACAUGAAGGCCAUCACCGCCA |
| | | | GGCGGAUCCUGCAGGGCCUGGGACACUACCUGAAGGCCGAGGGCAA |
| | | | GGUGGAGUGCUACAGGACCCUGCACCCCGUGCCACUGUACAGCUCC |
| | | | AGCGUGAACAGGGCCUUCUCCAGCCCCAAGGUGGCCGUGGAGGCCU |
| | | | GCAACGCUAUGCUGAAGGAGAACUUCCCCACCGUGGCCAGCUACUG |
| | | | CAUCAUCCCCGAGUACGACGCCUACCUGGACAUGGUGGACGGCGCC |
| | | | AGCUGCUGCCUGGACACCGCCAGCUUCUGCCCCGCCAAGCUGAGGA |
| | | | GCUUCCCCAAGAAACACAGCUACCUGGAGCCCACCAUCAGGAGCGC |
| | | | CGUGCCCAGCGCCAUCCAGAACACCCUGCAGAACGUGCUGGCCGCU |
| | | | GCCACCAAGAGGAACUGCAACGUGACCCAGAUGAGGGAGCUGCCCG |
| | | | UGCUGGACAGCGCUGCCUUCAACGUGGAGUGCUUCAAGAAAUACGC |
| | | | CUGCAACAACGAGUACUGGGAGACCUUCAAGGAGAACCCCAUCAGG |
| | | | CUGACCGAAGAGAACGUGGUGAACUACAUCACCAAGCUGAAGGGCC |
| | | | CCAAGGCCGCUGCCCUGUUCGCUAAGACCCACAACCUGAACAUGCU |
| | | | GCAGGACAUCCCAAUGGACAGGUUCGUGAUGGACCUGAAGAGGGAC |
| | | | GUGAAGGUGACACCCGGCACCAAGCACACCGAGGAGAGGCCCAAGG |
| | | | UGCAGGUGAUCCAGGCCGCUGACCCACUGGCCACCGCCUACCUGUG |
| | | | CGGCAUCCACAGGGAGCUGGUGAGGCGGCUGAACGCCGUGCUGCUG |
| | | | CCCAACAUCCACACCCUGUUCGACAUGAGCGCCGAGGACUUCGACG |
| | | | CCAUCAUCGCCGAGCACUUCCAGCCCGGCGACUGCGUGCUGGAGAC |
| | | | CGACAUCGCCAGCUUCGACAAGAGCGAGGAUGACGCUAUGGCCCUG |
| | | | ACCGCUCUGAUGAUCCUGGAGGACCUGGGCGUGGACGCCGAGCUGC |
| | | | UCACCCUGAUCGAGGCUGCCUUCGGCGAGAUCAGCUCCAUCCACCU |
| | | | GCCCACCAAGACCAAGUUCAAGUUCGGCGCUAUGAUGAAAAGCGGA |
| | | | AUGUUCCUGACCCUGUUCGUGAACACCGUGAUCAACAUUGUGAUCG |
| | | | CCAGCAGGGUGCUGCGGGAGAGGCUGACCGGCAGCCCCUGCGCUGC |
| | | | CUUCAUCGGCGACGACAACAUCGUGAAGGGCGUGAAAAGCGACAAG |
| | | | CUGAUGGCCGACAGGUGCGCCACCUGGCUGAACAUGGAGGUGAAGA |
| | | | UCAUCGACGCCGUGGUGGGCGAGAAGGCCCCCUACUUCUGCGGCGG |
| | | | AUUCAUCCUGUGCGACAGCGUGACCGGCACCGCCUGCAGGGUGGCC |
| | | | GACCCCCUGAAGAGGCUGUUCAAGCUGGGCAAGCCACUGGCCGCUG |
| | | | ACGAUGAGCACGACGAUGACAGGCGGAGGGCCCUGCACGAGGAAAG |
| | | | CACCAGGUGGAACAGGGUGGGCAUCCUGAGCGAGCUGUGCAAGGCC |
| | | | GUGGAGAGCAGGUACGAGACCGUGGGCACCAGCAUCAUCGUGAUGG |
| | | | CUAUGACCACACUGGCCAGCUCCGUCAAGAGCUUCUCCUACCUGAG |
| | | | GGGGGCCCCUAUAACUCUCUACGGCUAACCUGAAUGGACUACGACA |
| | | | UAGUCUAGUCCGCCAAGGCCGCCACCAUGAGAGUGACAGCCCCUAG |
| | | | AACCUUACUGCUUCUGCUUUGGGGAGCUGUUGCUCUGACAGAGACA |
| | | | UGGGCUGGAUCUCUGAGCGAGGUGACCGGCCAGGGCCUGUGCAUCG |
| | | | GCGCCGUGCCCAAGACCCACCAGGUGCUGUGCAACACCACCCAGAA |
| | | | GACCAGCGACGGCAGCUACUACCUGGCCGCUCCCACCGGCACCACC |
| | | | UGGGCCUGCAGCACCGGCCUGACCCCUUGCAUCAGCACCACCAUCC |
| | | | UGAACCUGACCACCGACUACUGCGUGCUGGUGGAGCUGUGGCCCAG |
| | | | GGUGACCUACCACAGCCCCAGCUACGCCUACCACCAGUUCGAGAGG |
| | | | AGGGCCAAGUACAAGAGGGAGCCCGUGAGCCUGACCCUGGCCCUGC |
| | | | UGCUGGGCGGCCUGACAAUGGGCGGCAUCGCCGCCGGCGUGGGCAC |
| | | | CGGCACCACCGCCCUGGUGGCCACCCAGCAGUUCCAGCAGCUGCAG |
| | | | GCCGCCAUGCACGACGACCUGAAGGAGGUGGAGAAGUCCAUCACCA |
| | | | ACCUGGAGAAGUCCCUGACCAGCCUGAGCGAGGUGGUGCUGCAGAA |
| | | | CAGGAGGGGCCUGGACCUGCUGUUCCUGAAGGAGGGCGGCCUGUGC |
| | | | GCCGCCCUGAAGGAGGAGUGCUGCCUGUACGCCGACCACACCGGCC |
| | | | UGGUGAUCGUGGGCAUUGUCGCUGGCCUGGCCGUCCUCGCCGUGGU |
| | | | GGUGAUUGGAGCUGUGGUCGCAGCUGUUAUGUGCAGAAGAAAGUCA |
| | | | UCCGGCGGAAAGGGAGGCUCCUACUCUCAGGCUGCUUCUGCUACAG |
| | | | UGCCUAGAGCUCUUAUGUGUUUAUCUCAGCUGGGCGGCGGAGGCAG |
| | | | CGACUACAAGGACGACGAUGACAAGUAAACUCGAGUAUGUUACGUG |
| | | | CAAAGGUGAUUGUCACCCCCCGAAAGACCAUAUUGUGACACACCCU |
| | | | CAGUAUCACGCCCAAACAUUUACAGCCGCGGUGUCAAAAACCGCGU |
| | | | GGACGUGGUUAACAUCCCUGCUGGGAGGAUCAGCCGUAAUUAUUAU |
| | | | AAUUGGCUUGGUGCUGGCUACUAUUGUGGCCAUGUACGUGCUGACC |
| | | | AACCAGAAACAUAAUUGAAUACAGCAGCAAUUGGCAAGCUGCUUAC |
| | | | AUAGAACUCGCGGCGAUUGGCAUGCCGCCUUAAAAUUUUUAUUUUA |
| | | | UUUUUUCUUUUCUUUUCCGAAUCGGAUUUUGUUUUUAAUAUUUCAA |
| | | | AAAAAAAAAAAAAAAAAAAAAAAUCUAGAAAAAAAAAAAAAAAAAA |
| | | | AAAAAAAAAAAAAAAAAAAAAAAAAAAAAAAAAAAAAAAAAAAAAA |
| | | | AAAAAAAAAAAAAAAAAAAAAAAAAAAAAAAAAAAA |
| 3068 | STARR^{TM} | 3068 | AUGGGCGGCGCAUGAGAGAAGCCCAGACCAAUUACCUACCCAAAAU |
| (SEQ | AH1A | | GGAGAAAGUUCACGUUGACAUCGAGGAAGACAGCCCAUUCCUCAGA |
| ID | 5- | | GCUUUGCAGCGGAGCUUCCCGCAGUUUGAGGUAGAAGCCAAGCAGG |
| NO: 108) | FLAG | | UCACUGAUAAUGACCAUGCUAAUGCCAGAGCGUUUUCGCAUCUGGC |
| | | | UUCAAAACUGAUCGAAACGGAGGUGGACCCAUCCGACACGAUCCUU |
| | | | GACAUUGGAAGUGCGCCCGCCCGCAGAAUGUAUUCUAAGCACAAGU |
| | | | AUCAUUGUAUCUGUCCGAUGAGAUGUGCGGAAGAUCCGGACAGAUU |
| | | | GUAUAAGUAUGCAACUAAGCUGAAGAAAAACUGUAAGGAAAUAACU |
| | | | GAUAAGGAAUUGGACAAGAAAAUGAAGGAGCUGGCCGCCGUCAUGA |
| | | | GCGACCCUGACCUGGAAACUGAGACUAUGUGCCUCCACGACGACGA |
| | | | GUCGUGUCGCUACGAAGGGCAAGUCGCUGUUUACCAGGAUGUAUAC |
| | | | GCCGUCGACGGCCCCACCAGCCUGUACCACCAGGCCAACAAGGGCG |
| | | | UGAGGGUGGCCUACUGGAUCGGCUUCGACACCACACCCUUCAUGUU |
| | | | CAAGAACCUGGCCGGCGCCUACCCCAGCUACAGCACCAACUGGGCC |
| | | | GACGAGACCGUGCUGACCGCCAGGAACAUCGGCCUGUGCAGCAGCG |
| | | | ACGUGAUGGAGAGGAGCCGGAGAGGCAUGAGCAUCCUGAGGAAGAA |
| | | | AUACCUGAAGCCCAGCAACAACGUGCUGUUCAGCGUGGGCAGCACC |
| | | | AUCUACCACGAGAAGAGGGACCUGCUCAGGAGCUGGCACCUGCCCA |
| | | | GCGUGUUCCACCUGAGGGGCAAGCAGAACUACACCUGCAGGUGCGA |
| | | | GACCAUCGUGAGCUGCGACGGCUACGUGGUGAAGAGGAUCGCCAUC |
| | | | AGCCCCGGCCUGUACGGCAAGCCCAGCGGCUACGCCGCUACAAUGC |
| | | | ACAGGGAGGGCUUCCUGUGCUGCAAGGUGACCGACACCCUGAACGG |
| | | | CGAGAGGGUGAGCUUCCCCGUGUGCACCUACGUGCCCGCCACCCUG |
| | | | UGCGACCAGAUGACCGGCAUCCUGGCCACCGACGUGAGCGCCGACG |
| | | | ACGCCCAGAAGCUGCUCGUGGGCCUGAACCAGAGGAUCGUGGUCAA |
| | | | CGGCAGGACCCAGAGGAACACCAACACAAUGAAGAACUACCUGCUG |
| | | | CCCGUGGUGGCCCAGGCUUUCGCCAGGUGGGCCAAGGAGUACAAGG |
| | | | AGGACCAGGAAGACGAGAGGCCCCUGGGCCUGAGGGACAGGCAGCU |
| | | | GGUGAUGGGCUGCUGCUGGGCCUUCAGGCGGCACAAGAUCACCAGC |
| | | | AUCUACAAGAGGCCCGACACCCAGACCAUCAUCAAGGUGAACAGCG |
| | | | ACUUCCACAGCUUCGUGCUGCCCAGGAUCGGCAGCAACACCCUGGA |
| | | | GAUCGGCCUGAGGACCCGGAUCAGGAAGAUGCUGGAGGAACACAAG |
| | | | GAGCCCAGCCCACUGAUCACCGCCGAGGACGUGCAGGAGGCCAAGU |
| | | | GCGCUGCCGACGAGGCCAAGGAGGUGAGGGAGGCCGAGGAACUGAG |
| | | | GGCCGCCCUGCCACCCCUGGCUGCCGACGUGGAGGAACCCACCCUG |
| | | | GAAGCCGACGUGGACCUGAUGCUGCAGGAGGCCGGCGCCGGAAGCG |
| | | | UGGAGACACCCAGGGGCCUGAUCAAGGUGACCAGCUACGACGGCGA |
| | | | GGACAAGAUCGGCAGCUACGCCGUGCUGAGCCCACAGGCCGUGCUG |
| | | | AAGUCCGAGAAGCUGAGCUGCAUCCACCCACUGGCCGAGCAGGUGA |
| | | | UCGUGAUCACCCACAGCGGCAGGAAGGGCAGGUACGCCGUGGAGCC |
| | | | CUACCACGGCAAGGUGGUCGUGCCCGAGGGCCACGCCAUCCCCGUG |
| | | | CAGGACUUCCAGGCCCUGAGCGAGAGCGCCACCAUCGUGUACAACG |
| | | | AGAGGGAGUUCGUGAACAGGUACCUGCACCAUAUCGCCACCCACGG |
| | | | CGGAGCCCUGAACACCGACGAGGAAUACUACAAGACCGUGAAGCCC |
| | | | AGCGAGCACGACGGCGAGUACCUGUACGACAUCGACAGGAAGCAGU |
| | | | GCGUGAAGAAAGAGCUGGUGACCGGCCUGGGACUGACCGGCGAGCU |
| | | | GGUGGACCCACCCUUCCACGAGUUCGCCUACGAGAGCCUGAGGACC |
| | | | AGACCCGCCGCUCCCUACCAGGUGCCCACCAUCGGCGUGUACGGCG |
| | | | UGCCCGGCAGCGGAAAGAGCGGCAUCAUCAAGAGCGCCGUGACCAA |
| | | | GAAAGACCUGGUGGUCAGCGCCAAGAAAGAGAACUGCGCCGAGAUC |
| | | | AUCAGGGACGUGAAGAAGAUGAAAGGCCUGGACGUGAACGCGCGCA |
| | | | CCGUGGACAGCGUGCUGCUGAACGGCUGCAAGCACCCCGUGGAGAC |
| | | | CCUGUACAUCGACGAGGCCUUCGCUUGCCACGCCGGCACCCUGAGG |
| | | | GCCCUGAUCGCCAUCAUCAGGCCCAAGAAAGCCGUGCUGUGCGGCG |
| | | | ACCCCAAGCAGUGCGGCUUCUUCAACAUGAUGUGCCUGAAGGUGCA |
| | | | CUUCAACCACGAGAUCUGCACCCAGGUGUUCCACAAGAGCAUCAGC |
| | | | AGGCGGUGCACCAAGAGCGUGACCAGCGUCGUGAGCACCCUGUUCU |
| | | | ACGACAAGAAAAUGAGGACCACCAACCCCAAGGAGACCAAAAUCGU |
| | | | GAUCGACACCACAGGCAGCACCAAGCCCAAGCAGGACGACCUGAUC |
| | | | CUGACCUGCUUCAGGGGCUGGGUGAAGCAGCUGCAGAUCGACUACA |
| | | | AGGGCAACGAGAUCAUGACCGCCGCUGCCAGCCAGGGCCUGACCAG |
| | | | GAAGGGCGUGUACGCCGUGAGGUACAAGGUGAACGAGAACCCACUG |
| | | | UACGCUCCCACCAGCGAGCACGUGAACGUGCUGCUGACCAGGACCG |
| | | | AGGACAGGAUCGUGUGGAAGACCCUGGCCGGCGACCCCUGGAUCAA |
| | | | GACCCUGACCGCCAAGUACCCCGGCAACUUCACCGCCACCAUCGAA |
| | | | GAGUGGCAGGCCGAGCACGACGCCAUCAUGAGGCACAUCCUGGAGA |
| | | | GGCCCGACCCCACCGACGUGUUCCAGAACAAGGCCAACGUGUGCUG |
| | | | GGCCAAGGCCCUGGUGCCCGUGCUGAAGACCGCCGGCAUCGACAUG |
| | | | ACCACAGAGCAGUGGAACACCGUGGACUACUUCGAGACCGACAAGG |
| | | | CCCACAGCGCCGAGAUCGUGCUGAACCAGCUGUGCGUGAGGUUCUU |
| | | | CGGCCUGGACCUGGACAGCGGCCUGUUCAGCGCCCCCACCGUGCCA |
| | | | CUGAGCAUCAGGAACAACCACUGGGACAACAGCCCCAGCCCAAACA |
| | | | UGUACGGCCUGAACAAGGAGGUGGUCAGGCAGCUGAGCAGGCGGUA |
| | | | CCCACAGCUGCCCAGGGCCGUGGCCACCGGCAGGGUGUACGACAUG |
| | | | AACACCGGCACCCUGAGGAACUACGACCCCAGGAUCAACCUGGUGC |
| | | | CCGUGAACAGGCGGCUGCCCCACGCCCUGGUGCUGCACCACAACGA |
| | | | GCACCCACAGAGCGACUUCAGCUCCUUCGUGAGCAAGCUGAAAGGC |
| | | | AGGACCGUGCUGGUCGUGGGCGAGAAGCUGAGCGUGCCCGGCAAGA |
| | | | UGGUGGACUGGCUGAGCGACAGGCCCGAGGCCACCUUCCGGGCCAG |
| | | | GCUGGACCUCGGCAUCCCCGGCGACGUGCCCAAGUACGACAUCAUC |
| | | | UUCGUGAACGUCAGGACCCCAUACAAGUACCACCAUUACCAGCAGU |
| | | | GCGAGGACCACGCCAUCAAGCUGAGCAUGCUGACCAAGAAGGCCUG |
| | | | CCUGCACCUGAACCCCGGAGGCACCUGCGUGAGCAUCGGCUACGGC |
| | | | UACGCCGACAGGGCCAGCGAGAGCAUCAUUGGCGCCAUCGCCAGGC |
| | | | UGUUCAAGUUCAGCAGGGUGUGCAAACCCAAGAGCAGCCUGGAGGA |
| | | | AACCGAGGUGCUGUUCGUGUUCAUCGGCUACGACCGGAAGGCCAGG |
| | | | ACCCACAACCCCUACAAGCUGAGCAGCACCCUGACAAACAUCUACA |
| | | | CCGGCAGCAGGCUGCACGAGGCCGGCUGCGCCCCCAGCUACCACGU |
| | | | GGUCAGGGGCGAUAUCGCCACCGCCACCGAGGGCGUGAUCAUCAAC |
| | | | GCUGCCAACAGCAAGGGCCAGCCCGGAGGCGGAGUGUGCGGCGCCC |
| | | | UGUACAAGAAGUUCCCCGAGAGCUUCGACCUGCAGCCCAUCGAGGU |
| | | | GGGCAAGGCCAGGCUGGUGAAGGGCGCCGCUAAGCACAUCAUCCAC |
| | | | GCCGUGGGCCCCAACUUCAACAAGGUGAGCGAGGUGGAAGGCGACA |
| | | | AGCAGCUGGCCGAAGCCUACGAGAGCAUCGCCAAGAUCGUGAACGA |
| | | | CAAUAACUACAAGAGCGUGGCCAUCCCACUGCUCAGCACCGGCAUC |
| | | | UUCAGCGGCAACAAGGACAGGCUGACCCAGAGCCUGAACCACCUGC |
| | | | UCACCGCCCUGGACACCACCGAUGCCGACGUGGCCAUCUACUGCAG |
| | | | GGACAAGAAGUGGGAGAUGACCCUGAAGGAGGCCGUGGCCAGGCGG |
| | | | GAGGCCGUGGAAGAGAUCUGCAUCAGCGACGACUCCAGCGUGACCG |
| | | | AGCCCGACGCCGAGCUGGUGAGGGUGCACCCCAAGAGCUCCCUGGC |
| | | | CGGCAGGAAGGGCUACAGCACCAGCGACGGCAAGACCUUCAGCUAC |
| | | | CUGGAGGGCACCAAGUUCCACCAGGCCGCUAAGGACAUCGCCGAGA |
| | | | UCAACGCUAUGUGGCCCGUGGCCACCGAGGCCAACGAGCAGGUGUG |
| | | | CAUGUACAUCCUGGGCGAGAGCAUGUCCAGCAUCAGGAGCAAGUGC |
| | | | CCCGUGGAGGAAAGCGAGGCCAGCACACCACCCAGCACCCUGCCCU |
| | | | GCCUGUGCAUCCACGCUAUGACACCCGAGAGGGUGCAGCGGCUGAA |
| | | | GGCCAGCAGGCCCGAGCAGAUCACCGUGUGCAGCUCCUUCCCACUG |
| | | | CCCAAGUACAGGAUCACCGGCGUGCAGAAGAUCCAGUGCAGCCAGC |
| | | | CCAUCCUGUUCAGCCCAAAGGUGCCCGCCUACAUCCACCCCAGGAA |
| | | | GUACCUGGUGGAGACCCCACCCGUGGACGAGACACCCGAGCCAAGC |
| | | | GCCGAGAACCAGAGCACCGAGGGCACACCCGAGCAGCCACCCCUGA |
| | | | UCACCGAGGACGAGACAAGGACCCGGACCCCAGAGCCCAUCAUUAU |
| | | | CGAGGAAGAGGAAGAGGACAGCAUCAGCCUGCUGAGCGACGGCCCC |
| | | | ACCCACCAGGUGCUGCAGGUGGAGGCCGACAUCCACGGCCCACCCA |
| | | | GCGUGUCCAGCUCCAGCUGGAGCAUCCCACACGCCAGCGACUUCGA |
| | | | CGUGGACAGCCUGAGCAUCCUGGACACCCUGGAGGGCGCCAGCGUG |
| | | | ACCUCCGGCGCCACCAGCGCCGAGACCAACAGCUACUUCGCCAAGA |
| | | | GCAUGGAGUUCCUGGCCAGGCCCGUGCCAGCUCCCAGGACCGUGUU |
| | | | CAGGAACCCACCCCACCCAGCUCCCAGGACCAGGACCCCAAGCCUG |
| | | | GCUCCCAGCAGGGCCUGCAGCAGGACCAGCCUGGUGAGCACCCCAC |
| | | | CCGGCGUGAACAGGGUGAUCACCAGGGAGGAACUGGAGGCCCUGAC |
| | | | ACCCAGCAGGACCCCCAGCAGGUCCGUGAGCAGGACUAGUCUGGUG |
| | | | UCCAACCCACCCGGCGUGAACAGGGUGAUCACCAGGGAGGAAUUCG |
| | | | AGGCCUUCGUGGCCCAGCAACAGAGACGGUUCGACGCCGGCGCCUA |
| | | | CAUCUUCAGCAGCGACACCGGCCAGGGACACCUGCAGCAAAAGAGC |
| | | | GUGAGGCAGACCGUGCUGAGCGAGGUGGUGCUGGAGAGGACCGAGC |
| | | | UGGAAAUCAGCUACGCCCCCAGGCUGGACCAGGAGAAGGAGGAACU |
| | | | GCUCAGGAAGAAACUGCAGCUGAACCCCACCCCAGCCAACAGGAGC |
| | | | AGGUACCAGAGCAGGAAGGUGGAGAACAUGAAGGCCAUCACCGCCA |
| | | | GGCGGAUCCUGCAGGGCCUGGGACACUACCUGAAGGCCGAGGGCAA |
| | | | GGUGGAGUGCUACAGGACCCUGCACCCCGUGCCACUGUACAGCUCC |
| | | | AGCGUGAACAGGGCCUUCUCCAGCCCCAAGGUGGCCGUGGAGGCCU |
| | | | GCAACGCUAUGCUGAAGGAGAACUUCCCCACCGUGGCCAGCUACUG |
| | | | CAUCAUCCCCGAGUACGACGCCUACCUGGACAUGGUGGACGGCGCC |
| | | | AGCUGCUGCCUGGACACCGCCAGCUUCUGCCCCGCCAAGCUGAGGA |
| | | | GCUUCCCCAAGAAACACAGCUACCUGGAGCCCACCAUCAGGAGCGC |
| | | | CGUGCCCAGCGCCAUCCAGAACACCCUGCAGAACGUGCUGGCCGCU |
| | | | GCCACCAAGAGGAACUGCAACGUGACCCAGAUGAGGGAGCUGCCCG |
| | | | UGCUGGACAGCGCUGCCUUCAACGUGGAGUGCUUCAAGAAAUACGC |
| | | | CUGCAACAACGAGUACUGGGAGACCUUCAAGGAGAACCCCAUCAGG |
| | | | CUGACCGAAGAGAACGUGGUGAACUACAUCACCAAGCUGAAGGGCC |
| | | | CCAAGGCCGCUGCCCUGUUCGCUAAGACCCACAACCUGAACAUGCU |
| | | | GCAGGACAUCCCAAUGGACAGGUUCGUGAUGGACCUGAAGAGGGAC |
| | | | GUGAAGGUGACACCCGGCACCAAGCACACCGAGGAGAGGCCCAAGG |
| | | | UGCAGGUGAUCCAGGCCGCUGACCCACUGGCCACCGCCUACCUGUG |
| | | | CGGCAUCCACAGGGAGCUGGUGAGGCGGCUGAACGCCGUGCUGCUG |
| | | | CCCAACAUCCACACCCUGUUCGACAUGAGCGCCGAGGACUUCGACG |
| | | | CCAUCAUCGCCGAGCACUUCCAGCCCGGCGACUGCGUGCUGGAGAC |
| | | | CGACAUCGCCAGCUUCGACAAGAGCGAGGAUGACGCUAUGGCCCUG |
| | | | ACCGCUCUGAUGAUCCUGGAGGACCUGGGCGUGGACGCCGAGCUGC |
| | | | UCACCCUGAUCGAGGCUGCCUUCGGCGAGAUCAGCUCCAUCCACCU |
| | | | GCCCACCAAGACCAAGUUCAAGUUCGGCGCUAUGAUGAAAAGCGGA |
| | | | AUGUUCCUGACCCUGUUCGUGAACACCGUGAUCAACAUUGUGAUCG |
| | | | CCAGCAGGGUGCUGCGGGAGAGGCUGACCGGCAGCCCCUGCGCUGC |
| | | | CUUCAUCGGCGACGACAACAUCGUGAAGGGCGUGAAAAGCGACAAG |
| | | | CUGAUGGCCGACAGGUGCGCCACCUGGCUGAACAUGGAGGUGAAGA |
| | | | UCAUCGACGCCGUGGUGGGCGAGAAGGCCCCCUACUUCUGCGGCGG |
| | | | AUUCAUCCUGUGCGACAGCGUGACCGGCACCGCCUGCAGGGUGGCC |
| | | | GACCCCCUGAAGAGGCUGUUCAAGCUGGGCAAGCCACUGGCCGCUG |
| | | | ACGAUGAGCACGACGAUGACAGGCGGAGGGCCCUGCACGAGGAAAG |
| | | | CACCAGGUGGAACAGGGUGGGCAUCCUGAGCGAGCUGUGCAAGGCC |
| | | | GUGGAGAGCAGGUACGAGACCGUGGGCACCAGCAUCAUCGUGAUGG |
| | | | CUAUGACCACACUGGCCAGCUCCGUCAAGAGCUUCUCCUACCUGAG |
| | | | GGGGGCCCCUAUAACUCUCUACGGCUAACCUGAAUGGACUACGACA |
| | | | UAGUCUAGUCCGCCAAGGCCGCCACCAUGAGAGUGACAGCCCCUAG |
| | | | AACCUUACUGCUUCUGCUUUGGGGAGCUGUUGCUCUGACAGAGACA |
| | | | UGGGCUGGAUCUUACCACAGCCCCAGCUACGCCUACCACCAGUUCG |
| | | | AGAGGGGGGGAGGAGGCUCCGGGGGAGGAGGCUCCCUGAAGAUCAG |
| | | | CCAGGCCGUGCACGCCGCCCACGCCGAGAUCAACGAGGCCGGCCGG |
| | | | GAGGUGAUCGUGGGCAUUGUCGCUGGCCUGGCCGUCCUCGCCGUGG |
| | | | UGGUGAUUGGAGCUGUGGUCGCAGCUGUUAUGUGCAGAAGAAAGUC |
| | | | AUCCGGCGGAAAGGGAGGCUCCUACUCUCAGGCUGCUUCUGCUACA |
| | | | GUGCCUAGAGCUCUUAUGUGUUUAUCUCAGCUGGGCGGCGGAGGCA |
| | | | GCGACUACAAGGACGACGAUGACAAGUAAACUCGAGUAUGUUACGU |
| | | | GCAAAGGUGAUUGUCACCCCCCGAAAGACCAUAUUGUGACACACCC |
| | | | UCAGUAUCACGCCCAAACAUUUACAGCCGCGGUGUCAAAAACCGCG |
| | | | UGGACGUGGUUAACAUCCCUGCUGGGAGGAUCAGCCGUAAUUAUUA |
| | | | UAAUUGGCUUGGUGCUGGCUACUAUUGUGGCCAUGUACGUGCUGAC |
| | | | CAACCAGAAACAUAAUUGAAUACAGCAGCAAUUGGCAAGCUGCUUA |
| | | | CAUAGAACUCGCGGCGAUUGGCAUGCCGCCUUAAAAUUUUUAUUUU |
| | | | AUUUUUUCUUUUCUUUUCCGAAUCGGAUUUUGUUUUUAAUAUUUCA |
| | | | AAAAAAAAAAAAAAAAAAAAAAAAUCUAGAAAAAAAAAAAAAAAAA |
| | | | AAAAAAAAAAAAAAAAAAAAAAAAAAAAAAAAAAAAAAAAAAAAAA |
| | | | AAAAAAAAAAAAAAAAAAAAAAAAAAAAAAAAAAAAA |

non structural protein of SINV

| mARM | | |
|---|---|---|
| # | | |
| 2842 | SINV | MEKPVVNVDVDPQSPFVVQLQKSFPQFEVVAQQVTPNDHANARAFS |
| and | nsP1- | HLASKLIELEVPTTATILDIGSAPARRMFSEHQYHCVCPMRSPEDP |
| 2862 | 4 AA | DRMMKYASKLAEKACKITNKNLHEKIKDLRTVLDTPDAETPSLCFH |
| (SEQ | | NDVTCNMRAEYSVMQDVYINAPGTIYHQAMKGVRTLYWIGFDTTQF |
| ID | | MFSAMAGSYPAYNTNWADEKVLEARNIGLCSTKLSEGRTGKLSIMR |
| NO: 109) | | KKELKPGSRVYFSVGSTLYPEHRASLQSWHLPSVFHLNGKQSYTCR |
| | | CDTVVSCEGYVVKKITISPGITGETVGYAVTHNSEGFLLCKVTDTV |
| | | KGERVSFPVCTYIPATICDQMTGIMATDISPDDAQKLLVGLNQRIV |
| | | INGRTNRNTNTMQNYLLPIIAQGFSKWAKERKDDLDNEKMLGTRER |
| | | KLTYGCLWAFRTKKVHSFYRPPGTQTCVKVPASFSAFPMSSVWTTS |
| | | LPMSLRQKLKLALQPKKEEKLLQVSEELVMEAKAAFEDAQEEARAE |
| | | KLREALPPLVADKGIEAAAEVVCEVEGLQADIGAALVETPRGHVRI |
| | | IPQANDRMIGQYIVVSPNSVLKNAKLAPAHPLADQVKIITHSGRSG |
| | | RYAVEPYDAKVLMPAGGAVPWPEFLALSESATLVYNEREFVNRKLY |
| | | HIAMHGPAKNTEEEQYKVTKAELAETEYVFDVDKKRCVKKEEASGL |
| | | VLSGELTNPPYHELALEGLKTRPAVPYKVETIGVIGTPGSGKSAII |
| | | KSTVTARDLVTSGKKENCREIEADVLRLRGMQITSKTVDSVMLNGC |
| | | HKAVEVLYVDEAFACHAGALLALIAIVRPRKKVVLCGDPMQCGFFN |
| | | MMQLKVHFNHPEKDICTKTFYKYISRRCTQPVTAIVSTLHYDGKMK |
| | | TTNPCKKNIEIDITGATKPKPGDIILTCFRGWVKQLQIDYPGHEVM |
| | | TAAASQGLTRKGVYAVRQKVNENPLYAITSEHVNVLLTRTEDRLVW |
| | | KTLQGDPWIKQLTNIPKGNFQATIEDWEAEHKGIIAAINSPTPRAN |
| | | PFSCKTNVCWAKALEPILATAGIVLTGCQWSELFPQFADDKPHSAI |
| | | YALDVICIKFFGMDLTSGLFSKQSIPLTYHPADSARPVAHWDNSPG |
| | | TRKYGYDHAIAAELSRRFPVFQLAGKGTQLDLQTGRTRVISAQHNL |
| | | VPVNRNLPHALVPEYKEKQPGPVEKFLNQFKHHSVLVVSEEKIEAP |
| | | RKRIEWIAPIGIAGADKNYNLAFGFPPQARYDLVFINIGTKYRNHH |
| | | FQQCEDHAATLKTLSRSALNCLNPGGTLVVKSYGYADRNSEDVVTA |
| | | LARKFVRVSAARPDCVSSNTEMYLIFRQLDNSRTRQFTPHHLNCVI |
| | | SSVYEGTRDGVGAAPSYRTKRENIADCQEEAVVNAANPLGRPGEGV |
| | | CRAIYKRWPTSFTDSATETGTARMTVCLGKKVIHAVGPDFRKHPEA |
| | | EALKLLQNAYHAVADLVNEHNIKSVAIPLLSTGIYAAGKDRLEVSL |
| | | NCLTTALDRTDADVTIYCLDKKWKERIDAALQLKESVTELKDEDME |
| | | IDDELVWIHPDSCLKGRKGFSTTKGKLYSYFEGTKFHQAAKDMAEI |
| | | KVLFPNDQESNEQLCAYILGETMEAIREKCPVDHNPSSSPPKTLPC |
| | | LCMYAMTPERVHRLRSNNVKEVTVCSSTPLPKHKIKNVQKVQCTKV |
| | | VLFNPHTPAFVPARKYIEVPEQPTAPPAQAEEAPEVVATPSPSTAD |
| | | NTSLDVTDISLDMDDSSEGSLFSSFSGSDNSITSMDSWSSGPSSLE |
| | | IVDRRQVVVADVHAVQEPAPIPPPRLKKMARLAAARKEPTPPASNS |
| | | SESLHLSFGGVSMSLGSIFDGETARQAAVQPLATGPTDVPMSFGSF |
| | | SDGEIDELSRRVTESEPVLFGSFEPGEVNSIISSRSAVSFPLRKQR |
| | | RRRRSRRTEY*LTGVGGYIFSTDTGPGHLQKKSVLQNQLTEPTLER |
| | | NVLERIHAPVLDTSKEEQLKLRYQMMPTEANKSRYQSRKVENQKAI |
| | | TTERLLSGLRLYNSATDQPECYKITYPKPLYSSSVPANYSDPQFAV |
| | | AVCNNYLHENYPTVASYQITDEYDAYLDMVDGTVACLDTATFCPAK |
| | | LRSYPKKHEYRAPNIRSAVPSAMQNTLQNVLIAATKRNCNVTQMRE |
| | | LPTLDSATFNVECFRKYACNDEYWEEFARKPIRITTEFVTAYVARL |
| | | KGPKAAALFAKTYNLVPLQEVPMDRFVMDMKRDVKVTPGTKHTEER |
| | | PKVQVIQAAEPLATAYICGIHRELVRRLTAVLLPNIHTLFDMSAED |
| | | FDAIIAEHFKQGDPVLETDIASFDKSQDDAMALTGLMILEDLGVDQ |
| | | PLLDLIECAFGEISSTHLPTGTRFKFGAMMKSGMFLTLFVNTVLNV |
| | | VIASRVLEERLKTSRCAAFIGDDNIIHGVVSDKEMAERCATWLNME |
| | | VKIIDAVIGERPPYFCGGFILQDSVTSTACRVADPLKRLFKLGKPL |
| | | PADDEQDEDRRRALLDETKAWFRVGITGTLAVAVTTRYEVDNITPV |
| | | LLALRTFAQSKRAFQAIRGEIKHLYGGPK |

### Example 2

This example describes analysis of the immunogenicity of influenza hemagglutinin (HA) expressed from self-replicating RNA or mRNA.

Self-replicating RNA and mRNA vaccine constructs were designed to encode the full-length hemagglutinin (HA) protein from influenza virus A/California/07/2009 (H1N1) (SEQ ID NO:113 and 114). The mRNA vaccine construct encoding HA included a tobacco etch virus (TEV) 5′ UTR and a Xenopus beta-globin (Xbg) 3′ UTR. Both self-replicating RNA (SEQ ID NO:56; entire RNA mARM3039) and mRNA vaccine constructs (SEQ ID NO:116; entire RNA sequence mARM3038) were encapsulated in the same lipid nanoparticle (LNP) composition that included four lipid excipients (an ionizable cationic lipid, 1,2-distearoyl-sn-glycero-3-phosphocholine (DSPC), cholesterol, and PEG2000-DMG) dispersed in HEPES buffer (pH 8.0) containing sodium chloride and the cryoprotectants sucrose and glycerol. The N:P ratio of complexing lipid and RNA was approximately 9:1. The ionizable cationic lipid had the following structure:

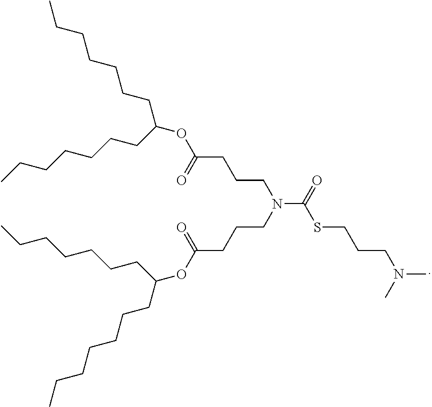 [SMILES: CCCCCCCC(CCCCCCC)OC(=O)CCCN(CCCC(=O)OC(CCCCCCC)CCCCCCC)C(=O)SCCCN(C)C]

Five female, 8-10 week old Balb/c mice were injected intramuscularly with 2 mg of mRNA or self-replicating RNA encoding HA. Mice were bled on days 14, 28, 42, and 56, followed by hemagglutination inhibition (HAI) assay using serially diluted sera. The reciprocal of the highest dilution of serum that caused inhibition of hemagglutination was considered the HAI titer, with a titer of 1/40 being protective against influenza virus infection and four-fold higher titers than baseline indicating seroconversion.

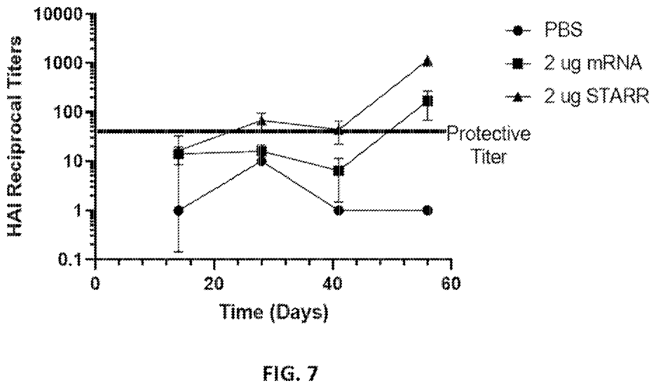

Results in FIG. 7 show that greater HAI titers were obtained with self-replicating RNA encoding HA as compared to mRNA encoding HA. HAI titers for the self-replicating RNA construct encoding HA were greater than HAI titers for the mRNA encoding HA at all time points beginning at day 28. In addition, protective HAI titers were seen for the self-replicating RNA construct encoding HA beginning at day 28 that were maintained for at least 56 days. By contrast, mRNA encoding HA showed protective HAI titers only at day 56 that were lower than HAI titers seen for the self-replicating RNA HA construct. At all other time points, HAI titers for the mRNA construct encoding HA were below the protective titer threshold, with an HAI titer that was comparable to injection with PBS control at day 28.

These results show that the self-replicating RNA construct encoding HA elicited protective HA antibody titers, with greater HAI titers as compared to the mRNA construct encoding HA.

### Example 3

This example describes dsRNA production and luciferase expression for self-replicating RNA.

Several self-replicating RNA systems from different alphaviruses were tested for expression in vitro using either green fluorescent protein (GFP) or firefly luciferase (Luc) as reporter genes. Initial transfection of cells with increasing amounts of self-replicating RNA resulted in expression of reporter genes at a lower dose compared to mRNA. However, as the amount of input self-replicating RNA increased, detectable expression of the reporter gene decreased.

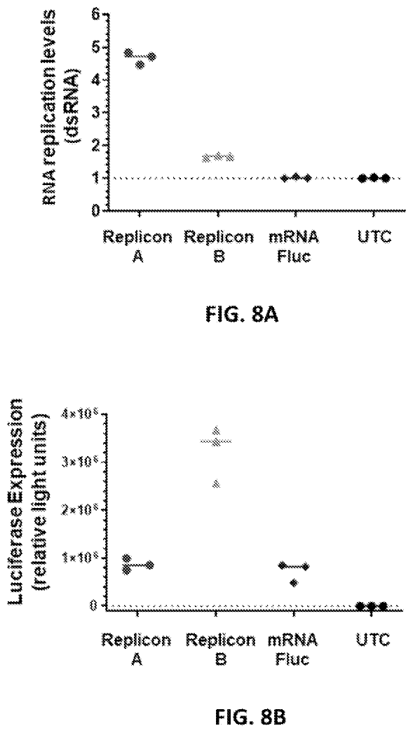

Self-replicating RNA produces double stranded RNA (dsRNA) as an intermediate in the amplification process. Overproduction of dsRNA can suppress translation. To evaluate the effect of dsRNA production on transgene expression, dsRNA and the expression of reporter gene luciferase were measured simultaneously. HEK293 cells were transfected with 2 μg of replicon A (SEQ ID NO:115; entire RNA sequence mARM2826) or replicon B (SEQ ID NO:100, entire RNA sequence mARM2809) self-replicating RNA, or mRNA expressing Luc (SEQ ID NO:102, entire mRNA sequence mARM1782) using a commercial RNA transfection reagent. Untransfected cells (UTC) served as a control. dsRNA production (FIG. 8A) was quantified using immunohistochemical staining for dsRNA, followed by fluorescence quantification using a fluorescence scanner 24 hours after transfection. Luciferase expression (FIG. 8B) was assayed by measuring bioluminescence in parallel.

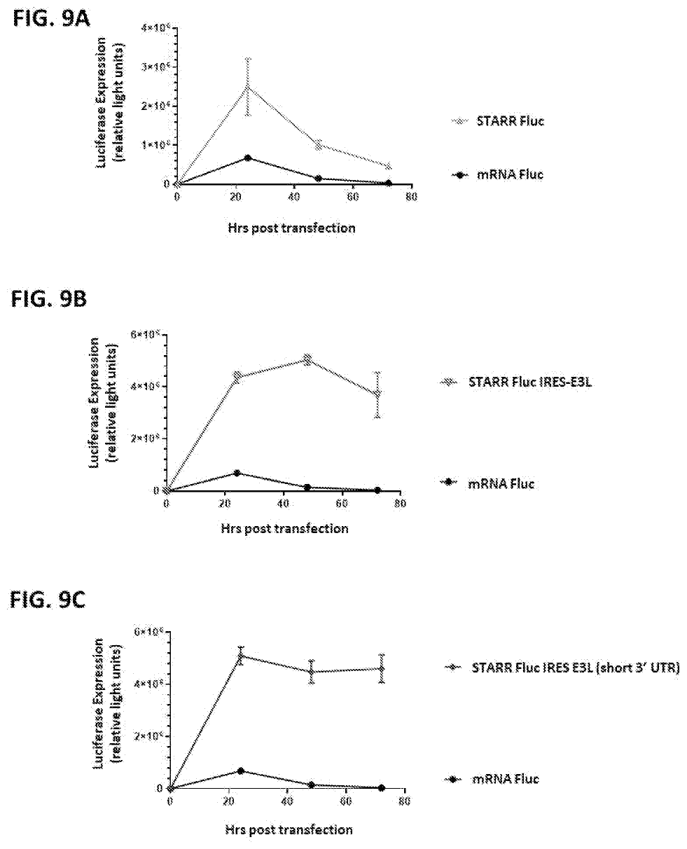

Replicon A produced a 3-fold higher level of dsRNA than replicon B 24 hrs after transfection (FIG. 8A). However, replicon B produced a 2.4-fold higher expression level of luciferase compared to replicon A. Furthermore, the level of luciferase expression from replicon A was equivalent to that observed for mRNA. Thus, even though replicon A had the ability to amplify the amount of replicon RNA and transcribed mRNA encoding luciferase, translation of the amplified mRNA was inhibited, consistent with overproduction of dsRNA inhibiting translation. Furthermore, higher levels of luciferase gene expression were seen for replicon RNA as compared to mRNA at 24, 48, and 72 hours after transfection of HEK293 cells (FIG. 9A). Self-replicating RNA with an expression cassette that included a luciferase reporter gene followed by an IRES and E3L also showed robust luciferase expression (FIGS. 9B, 9C; SEQ ID NOs: 118 and 119). Luciferase expression was also seen for a self-replicating RNA that expressed E3L from a first subgenomic promoter and a luciferase reporter gene from a second subgenomic promoter located 3′ of the E3L open reading frame (not shown). Thus, not only did replicon RNA produce higher levels of luciferase gene expression compared to mRNA, but replicon RNA also showed increased duration of expression over a 72-hr period.

### Example Sequences

Additional illustrative sequences are provided below, features of which are described in Table 7:

TABLE 7

| SEQ ID NO | Description |
|---|---|
| SEQ ID NO: 72 | nsP1-4 ORF, codon-optimized |
| SEQ ID NO: 73 | 5′ UTR |
| SEQ ID NO: 74 | 5′ UTR |
| SEQ ID NO: 75 | 5′ UTR |
| SEQ ID NO: 76 | 3′ UTR |
| SEQ ID NO: 77 | Intergenic region between nsP1-4 ORF and antigenic |
| | protein ORF |
| SEQ ID NO: 78 | Replicon sequence comprising SEQ ID NO: 72, SEQ |
| | ID NO: 73, SEQ ID NO: 76, and SEQ ID NO: 77 |
| SEQ ID NO: 79 | nsP1-4 protein sequence |
| SEQ ID NO: 80 | nsP1-4 protein sequence |
| SEQ ID NO: 81 | nsP1-4 protein sequence |
| SEQ ID NO: 82 | 5′ UTR (TEV) |
| SEQ ID NO: 83 | 3′ UTR (Xbg) |

SEQ ID NO: 72
ATGGAGAAAGTTCACGTTGACATCGAGGAAGACAGCCCATTCCTCAGAGCTTTG
CAGCGGAGCTTCCCGCAGTTTGAGGTAGAAGCCAAGCAGGTCACTGATAATGAC
CATGCTAATGCCAGAGCGTTTTCGCATCTGGCTTCAAAACTGATCGAAACGGAGG
TGGACCCATCCGACACGATCCTTGACATTGGAAGTGCGCCCGCCCGCAGAATGT
ATTCTAAGCACAAGTATCATTGTATCTGTCCGATGAGATGTGCGGAAGATCCGGA
CAGATTGTATAAGTATGCAACTAAGCTGAAGAAAAACTGTAAGGAAATAACTGA
TAAGGAATTGGACAAGAAAATGAAGGAGCTGGCCGCCGTCATGAGCGACCCTGA
CCTGGAAACTGAGACTATGTGCCTCCACGACGACGAGTCGTGTCGCTACGAAGG
GCAAGTCGCTGTTTACCAGGATGTATACGCCGTCGACGGCCCCACCAGCCTGTAC
CACCAGGCCAACAAGGGCGTGAGGGTGGCCTACTGGATCGGCTTCGACACCACA
CCCTTCATGTTCAAGAACCTGGCCGGCGCCTACCCCAGCTACAGCACCAACTGGG
CCGACGAGACCGTGCTGACCGCCAGGAACATCGGCCTGTGCAGCAGCGACGTGA
TGGAGAGGAGCCGGAGAGGCATGAGCATCCTGAGGAAGAAATACCTGAAGCCC
AGCAACAACGTGCTGTTCAGCGTGGGCAGCACCATCTACCACGAGAAGAGGGAC
CTGCTCAGGAGCTGGCACCTGCCCAGCGTGTTCCACCTGAGGGGCAAGCAGAAC
TACACCTGCAGGTGCGAGACCATCGTGAGCTGCGACGGCTACGTGGTGAAGAGG
ATCGCCATCAGCCCCGGCCTGTACGGCAAGCCCAGCGGCTACGCCGCTACAATG
CACAGGGAGGGCTTCCTGTGCTGCAAGGTGACCGACACCCTGAACGGCGAGAGG
GTGAGCTTCCCCGTGTGCACCTACGTGCCCGCCACCCTGTGCGACCAGATGACCG
GCATCCTGGCCACCGACGTGAGCGCCGACGACGCCCAGAAGCTGCTCGTGGGCC
TGAACCAGAGGATCGTGGTCAACGGCAGGACCCAGAGGAACACCAACACAATG
AAGAACTACCTGCTGCCCGTGGTGGCCCAGGCTTTCGCCAGGTGGGCCAAGGAG
TACAAGGAGGACCAGGAAGACGAGAGGCCCCTGGGCCTGAGGGACAGGCAGCT
GGTGATGGGCTGCTGCTGGGCCTTCAGGCGGCACAAGATCACCAGCATCTACAA
GAGGCCCGACACCCAGACCATCATCAAGGTGAACAGCGACTTCCACAGCTTCGT
GCTGCCCAGGATCGGCAGCAACACCCTGGAGATCGGCCTGAGGACCCGGATCAG
GAAGATGCTGGAGGAACACAAGGAGCCCAGCCCACTGATCACCGCCGAGGACGT
GCAGGAGGCCAAGTGCGCTGCCGACGAGGCCAAGGAGGTGAGGGAGGCCGAGG
AACTGAGGGCCGCCCTGCCACCCCTGGCTGCCGACGTGGAGGAACCCACCCTGG
AAGCCGACGTGGACCTGATGCTGCAGGAGGCCGGCGCCGGAAGCGTGGAGACA
CCCAGGGGCCTGATCAAGGTGACCAGCTACGACGGCGAGGACAAGATCGGCAGC
TACGCCGTGCTGAGCCCACAGGCCGTGCTGAAGTCCGAGAAGCTGAGCTGCATC
CACCCACTGGCCGAGCAGGTGATCGTGATCACCCACAGCGGCAGGAAGGGCAGG
TACGCCGTGGAGCCCTACCACGGCAAGGTGGTCGTGCCCGAGGGCCACGCCATC
CCCGTGCAGGACTTCCAGGCCCTGAGCGAGAGCGCCACCATCGTGTACAACGAG
AGGGAGTTCGTGAACAGGTACCTGCACCATATCGCCACCCACGGCGGAGCCCTG
AACACCGACGAGGAATACTACAAGACCGTGAAGCCCAGCGAGCACGACGGCGA
GTACCTGTACGACATCGACAGGAAGCAGTGCGTGAAGAAAGAGCTGGTGACCGG
CCTGGGACTGACCGGCGAGCTGGTGGACCCACCCTTCCACGAGTTCGCCTACGA
GAGCCTGAGGACCAGACCCGCCGCTCCCTACCAGGTGCCCACCATCGGCGTGTA
CGGCGTGCCCGGCAGCGGAAAGAGCGGCATCATCAAGAGCGCCGTGACCAAGA
AAGACCTGGTGGTCAGCGCCAAGAAAGAGAACTGCGCCGAGATCATCAGGGAC
GTGAAGAAGATGAAAGGCCTGGACGTGAACGCGCGCACCGTGGACAGCGTGCTG
CTGAACGGCTGCAAGCACCCCGTGGAGACCCTGTACATCGACGAGGCCTTCGCTT
GCCACGCCGGCACCCTGAGGGCCCTGATCGCCATCATCAGGCCCAAGAAAGCCG
TGCTGTGCGGCGACCCCAAGCAGTGCGGCTTCTTCAACATGATGTGCCTGAAGGT
GCACTTCAACCACGAGATCTGCACCCAGGTGTTCCACAAGAGCATCAGCAGGCG
GTGCACCAAGAGCGTGACCAGCGTCGTGAGCACCCTGTTCTACGACAAGAAAAT
GAGGACCACCAACCCCAAGGAGACCAAAATCGTGATCGACACCACAGGCAGCA
CCAAGCCCAAGCAGGACGACCTGATCCTGACCTGCTTCAGGGGCTGGGTGAAGC
AGCTGCAGATCGACTACAAGGGCAACGAGATCATGACCGCCGCTGCCAGCCAGG
GCCTGACCAGGAAGGGCGTGTACGCCGTGAGGTACAAGGTGAACGAGAACCCAC
TGTACGCTCCCACCAGCGAGCACGTGAACGTGCTGCTGACCAGGACCGAGGACA
GGATCGTGTGGAAGACCCTGGCCGGCGACCCCTGGATCAAGACCCTGACCGCCA
AGTACCCCGGCAACTTCACCGCCACCATCGAAGAGTGGCAGGCCGAGCACGACG
CCATCATGAGGCACATCCTGGAGAGGCCCGACCCCACCGACGTGTTCCAGAACA
AGGCCAACGTGTGCTGGGCCAAGGCCCTGGTGCCCGTGCTGAAGACCGCCGGCA
TCGACATGACCACAGAGCAGTGGAACACCGTGGACTACTTCGAGACCGACAAGG
CCCACAGCGCCGAGATCGTGCTGAACCAGCTGTGCGTGAGGTTCTTCGGCCTGGA
CCTGGACAGCGGCCTGTTCAGCGCCCCCACCGTGCCACTGAGCATCAGGAACAA
CCACTGGGACAACAGCCCCAGCCCAAACATGTACGGCCTGAACAAGGAGGTGGT
CAGGCAGCTGAGCAGGCGGTACCCACAGCTGCCCAGGGCCGTGGCCACCGGCAG
GGTGTACGACATGAACACCGGCACCCTGAGGAACTACGACCCCAGGATCAACCT
GGTGCCCGTGAACAGGCGGCTGCCCCACGCCCTGGTGCTGCACCACAACGAGCA
CCCACAGAGCGACTTCAGCTCCTTCGTGAGCAAGCTGAAAGGCAGGACCGTGCT
GGTCGTGGGCGAGAAGCTGAGCGTGCCCGGCAAGATGGTGGACTGGCTGAGCGA
CAGGCCCGAGGCCACCTTCCGGGCCAGGCTGGACCTCGGCATCCCCGGCGACGT
GCCCAAGTACGACATCATCTTCGTGAACGTCAGGACCCCATACAAGTACCACCAT
TACCAGCAGTGCGAGGACCACGCCATCAAGCTGAGCATGCTGACCAAGAAGGCC
TGCCTGCACCTGAACCCCGGAGGCACCTGCGTGAGCATCGGCTACGGCTACGCC
GACAGGGCCAGCGAGAGCATCATTGGCGCCATCGCCAGGCTGTTCAAGTTCAGC
AGGGTGTGCAAACCCAAGAGCAGCCTGGAGGAAACCGAGGTGCTGTTCGTGTTC
ATCGGCTACGACCGGAAGGCCAGGACCCACAACCCCTACAAGCTGAGCAGCACC
CTGACAAACATCTACACCGGCAGCAGGCTGCACGAGGCCGGCTGCGCCCCCAGC
TACCACGTGGTCAGGGGCGATATCGCCACCGCCACCGAGGGCGTGATCATCAAC
GCTGCCAACAGCAAGGGCCAGCCCGGAGGCGGAGTGTGCGGCGCCCTGTACAAG
AAGTTCCCCGAGAGCTTCGACCTGCAGCCCATCGAGGTGGGCAAGGCCAGGCTG
GTGAAGGGCGCCGCTAAGCACATCATCCACGCCGTGGGCCCCAACTTCAACAAG
GTGAGCGAGGTGGAAGGCGACAAGCAGCTGGCCGAAGCCTACGAGAGCATCGC
CAAGATCGTGAACGACAATAACTACAAGAGCGTGGCCATCCCACTGCTCAGCAC
CGGCATCTTCAGCGGCAACAAGGACAGGCTGACCCAGAGCCTGAACCACCTGCT
CACCGCCCTGGACACCACCGATGCCGACGTGGCCATCTACTGCAGGGACAAGAA
GTGGGAGATGACCCTGAAGGAGGCCGTGGCCAGGCGGGAGGCCGTGGAAGAGA
TCTGCATCAGCGACGACTCCAGCGTGACCGAGCCCGACGCCGAGCTGGTGAGGG
TGCACCCCAAGAGCTCCCTGGCCGGCAGGAAGGGCTACAGCACCAGCGACGGCA
AGACCTTCAGCTACCTGGAGGGCACCAAGTTCCACCAGGCCGCTAAGGACATCG
CCGAGATCAACGCTATGTGGCCCGTGGCCACCGAGGCCAACGAGCAGGTGTGCA
TGTACATCCTGGGCGAGAGCATGTCCAGCATCAGGAGCAAGTGCCCCGTGGAGG
AAAGCGAGGCCAGCACACCACCCAGCACCCTGCCCTGCCTGTGCATCCACGCTA
TGACACCCGAGAGGGTGCAGCGGCTGAAGGCCAGCAGGCCCGAGCAGATCACC
GTGTGCAGCTCCTTCCCACTGCCCAAGTACAGGATCACCGGCGTGCAGAAGATCC
AGTGCAGCCAGCCCATCCTGTTCAGCCCAAAGGTGCCCGCCTACATCCACCCCAG
GAAGTACCTGGTGGAGACCCCACCCGTGGACGAGACACCCGAGCCAAGCGCCGA
GAACCAGAGCACCGAGGGCACACCCGAGCAGCCACCCCTGATCACCGAGGACG
AGACAAGGACCCGGACCCCAGAGCCCATCATTATCGAGGAAGAGGAAGAGGAC
AGCATCAGCCTGCTGAGCGACGGCCCCACCCACCAGGTGCTGCAGGTGGAGGCC
GACATCCACGGCCCACCCAGCGTGTCCAGCTCCAGCTGGAGCATCCCACACGCC
AGCGACTTCGACGTGGACAGCCTGAGCATCCTGGACACCCTGGAGGGCGCCAGC
GTGACCTCCGGCGCCACCAGCGCCGAGACCAACAGCTACTTCGCCAAGAGCATG
GAGTTCCTGGCCAGGCCCGTGCCAGCTCCCAGGACCGTGTTCAGGAACCCACCCC
ACCCAGCTCCCAGGACCAGGACCCCAAGCCTGGCTCCCAGCAGGGCCTGCAGCA
GGACCAGCCTGGTGAGCACCCCACCCGGCGTGAACAGGGTGATCACCAGGGAGG
AACTGGAGGCCCTGACACCCAGCAGGACCCCCAGCAGGTCCGTGAGCAGGACTA
GTCTGGTGTCCAACCCACCCGGCGTGAACAGGGTGATCACCAGGGAGGAATTCG
AGGCCTTCGTGGCCCAGCAACAGAGACGGTTCGACGCCGGCGCCTACATCTTCA
GCAGCGACACCGGCCAGGGACACCTGCAGCAAAAGAGCGTGAGGCAGACCGTG
CTGAGCGAGGTGGTGCTGGAGAGGACCGAGCTGGAAATCAGCTACGCCCCCAGG
CTGGACCAGGAGAAGGAGGAACTGCTCAGGAAGAAACTGCAGCTGAACCCCAC
CCCAGCCAACAGGAGCAGGTACCAGAGCAGGAAGGTGGAGAACATGAAGGCCA
TCACCGCCAGGCGGATCCTGCAGGGCCTGGGACACTACCTGAAGGCCGAGGGCA
AGGTGGAGTGCTACAGGACCCTGCACCCCGTGCCACTGTACAGCTCCAGCGTGA
ACAGGGCCTTCTCCAGCCCCAAGGTGGCCGTGGAGGCCTGCAACGCTATGCTGA
AGGAGAACTTCCCCACCGTGGCCAGCTACTGCATCATCCCCGAGTACGACGCCTA
CCTGGACATGGTGGACGGCGCCAGCTGCTGCCTGGACACCGCCAGCTTCTGCCCC
GCCAAGCTGAGGAGCTTCCCCAAGAAACACAGCTACCTGGAGCCCACCATCAGG
AGCGCCGTGCCCAGCGCCATCCAGAACACCCTGCAGAACGTGCTGGCCGCTGCC
ACCAAGAGGAACTGCAACGTGACCCAGATGAGGGAGCTGCCCGTGCTGGACAGC
GCTGCCTTCAACGTGGAGTGCTTCAAGAAATACGCCTGCAACAACGAGTACTGG
GAGACCTTCAAGGAGAACCCCATCAGGCTGACCGAAGAGAACGTGGTGAACTAC
ATCACCAAGCTGAAGGGCCCCAAGGCCGCTGCCCTGTTCGCTAAGACCCACAAC
CTGAACATGCTGCAGGACATCCCAATGGACAGGTTCGTGATGGACCTGAAGAGG
GACGTGAAGGTGACACCCGGCACCAAGCACACCGAGGAGAGGCCCAAGGTGCA
GGTGATCCAGGCCGCTGACCCACTGGCCACCGCCTACCTGTGCGGCATCCACAG
GGAGCTGGTGAGGCGGCTGAACGCCGTGCTGCTGCCCAACATCCACACCCTGTTC
GACATGAGCGCCGAGGACTTCGACGCCATCATCGCCGAGCACTTCCAGCCCGGC
GACTGCGTGCTGGAGACCGACATCGCCAGCTTCGACAAGAGCGAGGATGACGCT
ATGGCCCTGACCGCTCTGATGATCCTGGAGGACCTGGGCGTGGACGCCGAGCTG
CTCACCCTGATCGAGGCTGCCTTCGGCGAGATCAGCTCCATCCACCTGCCCACCA
AGACCAAGTTCAAGTTCGGCGCTATGATGAAAAGCGGAATGTTCCTGACCCTGTT
CGTGAACACCGTGATCAACATTGTGATCGCCAGCAGGGTGCTGCGGGAGAGGCT
GACCGGCAGCCCCTGCGCTGCCTTCATCGGCGACGACAACATCGTGAAGGGCGT
GAAAAGCGACAAGCTGATGGCCGACAGGTGCGCCACCTGGCTGAACATGGAGGT
GAAGATCATCGACGCCGTGGTGGGCGAGAAGGCCCCCTACTTCTGCGGCGGATT
CATCCTGTGCGACAGCGTGACCGGCACCGCCTGCAGGGTGGCCGACCCCCTGAA
GAGGCTGTTCAAGCTGGGCAAGCCACTGGCCGCTGACGATGAGCACGACGATGA
CAGGCGGAGGGCCCTGCACGAGGAAAGCACCAGGTGGAACAGGGTGGGCATCC
TGAGCGAGCTGTGCAAGGCCGTGGAGAGCAGGTACGAGACCGTGGGCACCAGC
ATCATCGTGATGGCTATGACCACACTGGCCAGCTCCGTCAAGAGCTTCTCCTACC
TGAGGGGGGCCCCTATAACTCTCTACGGCTAA
SEQ ID NO: 73
ATGGGCGGCGCATGAGAGAAGCCCAGACCAATTACCTACCCAAA
SEQ ID NO: 74
GATGGGCGGCGCATGAGAGAAGCCCAGACCAATTACCTACCCAAA
SEQ ID NO: 75
GATAGGCGGCGCATGAGAGAAGCCCAGACCAATTACCTACCCAAA
SEQ ID NO: 76
ACTCGAGTATGTTACGTGCAAAGGTGATTGTCACCCCCCGAAAGACCATATTGTG
ACACACCCTCAGTATCACGCCCAAACATTTACAGCCGCGGTGTCAAAAACCGCG
TGGACGTGGTTAACATCCCTGCTGGGAGGATCAGCCGTAATTATTATAATTGGCT
TGGTGCTGGCTACTATTGTGGCCATGTACGTGCTGACCAACCAGAAACATAATTG
AATACAGCAGCAATTGGCAAGCTGCTTACATAGAACTCGCGGCGATTGGCATGC
CGCCTTAAAATTTTTATTTTATTTTTTCTTTTCTTTTCCGAATCGGATTTTGTTTTTA
ATATTTCAAAAAAAAAAAAAAAAAAAAAAAAATCTAGAAAAAAAAAAAAAAAA
AAAAAAAAAAAAAAAAAAAAAAAAAAAAAAAAAAAAAAAAAAAAAAAAAAAA
AAAAAAAAAAAAAAAAAAAAAAAAAAAAAAAA
SEQ ID NO: 77
CCTGAATGGACTACGACATAGTCTAGTCCGCCAAGGCCGCCACC
SEQ ID NO: 78
ATGGGCGGCGCATGAGAGAAGCCCAGACCAATTACCTACCCAAAATGGAGAAA
GTTCACGTTGACATCGAGGAAGACAGCCCATTCCTCAGAGCTTTGCAGCGGAGCT
TCCCGCAGTTTGAGGTAGAAGCCAAGCAGGTCACTGATAATGACCATGCTAATG
CCAGAGCGTTTTCGCATCTGGCTTCAAAACTGATCGAAACGGAGGTGGACCCATC
CGACACGATCCTTGACATTGGAAGTGCGCCCGCCCGCAGAATGTATTCTAAGCAC
AAGTATCATTGTATCTGTCCGATGAGATGTGCGGAAGATCCGGACAGATTGTATA
AGTATGCAACTAAGCTGAAGAAAAACTGTAAGGAAATAACTGATAAGGAATTGG
ACAAGAAAATGAAGGAGCTGGCCGCCGTCATGAGCGACCCTGACCTGGAAACTG
AGACTATGTGCCTCCACGACGACGAGTCGTGTCGCTACGAAGGGCAAGTCGCTG
TTTACCAGGATGTATACGCCGTCGACGGCCCCACCAGCCTGTACCACCAGGCCAA
CAAGGGCGTGAGGGTGGCCTACTGGATCGGCTTCGACACCACACCCTTCATGTTC
AAGAACCTGGCCGGCGCCTACCCCAGCTACAGCACCAACTGGGCCGACGAGACC
GTGCTGACCGCCAGGAACATCGGCCTGTGCAGCAGCGACGTGATGGAGAGGAGC
CGGAGAGGCATGAGCATCCTGAGGAAGAAATACCTGAAGCCCAGCAACAACGT
GCTGTTCAGCGTGGGCAGCACCATCTACCACGAGAAGAGGGACCTGCTCAGGAG
CTGGCACCTGCCCAGCGTGTTCCACCTGAGGGGCAAGCAGAACTACACCTGCAG
GTGCGAGACCATCGTGAGCTGCGACGGCTACGTGGTGAAGAGGATCGCCATCAG
CCCCGGCCTGTACGGCAAGCCCAGCGGCTACGCCGCTACAATGCACAGGGAGGG
CTTCCTGTGCTGCAAGGTGACCGACACCCTGAACGGCGAGAGGGTGAGCTTCCC
CGTGTGCACCTACGTGCCCGCCACCCTGTGCGACCAGATGACCGGCATCCTGGCC
ACCGACGTGAGCGCCGACGACGCCCAGAAGCTGCTCGTGGGCCTGAACCAGAGG
ATCGTGGTCAACGGCAGGACCCAGAGGAACACCAACACAATGAAGAACTACCTG
CTGCCCGTGGTGGCCCAGGCTTTCGCCAGGTGGGCCAAGGAGTACAAGGAGGAC
CAGGAAGACGAGAGGCCCCTGGGCCTGAGGGACAGGCAGCTGGTGATGGGCTG
CTGCTGGGCCTTCAGGCGGCACAAGATCACCAGCATCTACAAGAGGCCCGACAC
CCAGACCATCATCAAGGTGAACAGCGACTTCCACAGCTTCGTGCTGCCCAGGATC
GGCAGCAACACCCTGGAGATCGGCCTGAGGACCCGGATCAGGAAGATGCTGGAG
GAACACAAGGAGCCCAGCCCACTGATCACCGCCGAGGACGTGCAGGAGGCCAA
GTGCGCTGCCGACGAGGCCAAGGAGGTGAGGGAGGCCGAGGAACTGAGGGCCG
CCCTGCCACCCCTGGCTGCCGACGTGGAGGAACCCACCCTGGAAGCCGACGTGG
ACCTGATGCTGCAGGAGGCCGGCGCCGGAAGCGTGGAGACACCCAGGGGCCTGA
TCAAGGTGACCAGCTACGACGGCGAGGACAAGATCGGCAGCTACGCCGTGCTGA
GCCCACAGGCCGTGCTGAAGTCCGAGAAGCTGAGCTGCATCCACCCACTGGCCG
AGCAGGTGATCGTGATCACCCACAGCGGCAGGAAGGGCAGGTACGCCGTGGAGC
CCTACCACGGCAAGGTGGTCGTGCCCGAGGGCCACGCCATCCCCGTGCAGGACT
TCCAGGCCCTGAGCGAGAGCGCCACCATCGTGTACAACGAGAGGGAGTTCGTGA
ACAGGTACCTGCACCATATCGCCACCCACGGCGGAGCCCTGAACACCGACGAGG
AATACTACAAGACCGTGAAGCCCAGCGAGCACGACGGCGAGTACCTGTACGACA
TCGACAGGAAGCAGTGCGTGAAGAAAGAGCTGGTGACCGGCCTGGGACTGACCG
GCGAGCTGGTGGACCCACCCTTCCACGAGTTCGCCTACGAGAGCCTGAGGACCA
GACCCGCCGCTCCCTACCAGGTGCCCACCATCGGCGTGTACGGCGTGCCCGGCA
GCGGAAAGAGCGGCATCATCAAGAGCGCCGTGACCAAGAAAGACCTGGTGGTC
AGCGCCAAGAAAGAGAACTGCGCCGAGATCATCAGGGACGTGAAGAAGATGAA
AGGCCTGGACGTGAACGCGCGCACCGTGGACAGCGTGCTGCTGAACGGCTGCAA
GCACCCCGTGGAGACCCTGTACATCGACGAGGCCTTCGCTTGCCACGCCGGCACC
CTGAGGGCCCTGATCGCCATCATCAGGCCCAAGAAAGCCGTGCTGTGCGGCGAC
CCCAAGCAGTGCGGCTTCTTCAACATGATGTGCCTGAAGGTGCACTTCAACCACG
AGATCTGCACCCAGGTGTTCCACAAGAGCATCAGCAGGCGGTGCACCAAGAGCG
TGACCAGCGTCGTGAGCACCCTGTTCTACGACAAGAAAATGAGGACCACCAACC
CCAAGGAGACCAAAATCGTGATCGACACCACAGGCAGCACCAAGCCCAAGCAG
GACGACCTGATCCTGACCTGCTTCAGGGGCTGGGTGAAGCAGCTGCAGATCGAC
TACAAGGGCAACGAGATCATGACCGCCGCTGCCAGCCAGGGCCTGACCAGGAAG
GGCGTGTACGCCGTGAGGTACAAGGTGAACGAGAACCCACTGTACGCTCCCACC
AGCGAGCACGTGAACGTGCTGCTGACCAGGACCGAGGACAGGATCGTGTGGAAG
ACCCTGGCCGGCGACCCCTGGATCAAGACCCTGACCGCCAAGTACCCCGGCAAC
TTCACCGCCACCATCGAAGAGTGGCAGGCCGAGCACGACGCCATCATGAGGCAC
ATCCTGGAGAGGCCCGACCCCACCGACGTGTTCCAGAACAAGGCCAACGTGTGC
TGGGCCAAGGCCCTGGTGCCCGTGCTGAAGACCGCCGGCATCGACATGACCACA
GAGCAGTGGAACACCGTGGACTACTTCGAGACCGACAAGGCCCACAGCGCCGAG
ATCGTGCTGAACCAGCTGTGCGTGAGGTTCTTCGGCCTGGACCTGGACAGCGGCC
TGTTCAGCGCCCCCACCGTGCCACTGAGCATCAGGAACAACCACTGGGACAACA
GCCCCAGCCCAAACATGTACGGCCTGAACAAGGAGGTGGTCAGGCAGCTGAGCA
GGCGGTACCCACAGCTGCCCAGGGCCGTGGCCACCGGCAGGGTGTACGACATGA
ACACCGGCACCCTGAGGAACTACGACCCCAGGATCAACCTGGTGCCCGTGAACA
GGCGGCTGCCCCACGCCCTGGTGCTGCACCACAACGAGCACCCACAGAGCGACT
TCAGCTCCTTCGTGAGCAAGCTGAAAGGCAGGACCGTGCTGGTCGTGGGCGAGA
AGCTGAGCGTGCCCGGCAAGATGGTGGACTGGCTGAGCGACAGGCCCGAGGCCA
CCTTCCGGGCCAGGCTGGACCTCGGCATCCCCGGCGACGTGCCCAAGTACGACA
TCATCTTCGTGAACGTCAGGACCCCATACAAGTACCACCATTACCAGCAGTGCGA
GGACCACGCCATCAAGCTGAGCATGCTGACCAAGAAGGCCTGCCTGCACCTGAA
CCCCGGAGGCACCTGCGTGAGCATCGGCTACGGCTACGCCGACAGGGCCAGCGA
GAGCATCATTGGCGCCATCGCCAGGCTGTTCAAGTTCAGCAGGGTGTGCAAACC
CAAGAGCAGCCTGGAGGAAACCGAGGTGCTGTTCGTGTTCATCGGCTACGACCG
GAAGGCCAGGACCCACAACCCCTACAAGCTGAGCAGCACCCTGACAAACATCTA
CACCGGCAGCAGGCTGCACGAGGCCGGCTGCGCCCCCAGCTACCACGTGGTCAG
GGGCGATATCGCCACCGCCACCGAGGGCGTGATCATCAACGCTGCCAACAGCAA
GGGCCAGCCCGGAGGCGGAGTGTGCGGCGCCCTGTACAAGAAGTTCCCCGAGAG
CTTCGACCTGCAGCCCATCGAGGTGGGCAAGGCCAGGCTGGTGAAGGGCGCCGC
TAAGCACATCATCCACGCCGTGGGCCCCAACTTCAACAAGGTGAGCGAGGTGGA
AGGCGACAAGCAGCTGGCCGAAGCCTACGAGAGCATCGCCAAGATCGTGAACG
ACAATAACTACAAGAGCGTGGCCATCCCACTGCTCAGCACCGGCATCTTCAGCG
GCAACAAGGACAGGCTGACCCAGAGCCTGAACCACCTGCTCACCGCCCTGGACA
CCACCGATGCCGACGTGGCCATCTACTGCAGGGACAAGAAGTGGGAGATGACCC
TGAAGGAGGCCGTGGCCAGGCGGGAGGCCGTGGAAGAGATCTGCATCAGCGAC
GACTCCAGCGTGACCGAGCCCGACGCCGAGCTGGTGAGGGTGCACCCCAAGAGC
TCCCTGGCCGGCAGGAAGGGCTACAGCACCAGCGACGGCAAGACCTTCAGCTAC
CTGGAGGGCACCAAGTTCCACCAGGCCGCTAAGGACATCGCCGAGATCAACGCT
ATGTGGCCCGTGGCCACCGAGGCCAACGAGCAGGTGTGCATGTACATCCTGGGC
GAGAGCATGTCCAGCATCAGGAGCAAGTGCCCCGTGGAGGAAAGCGAGGCCAG
CACACCACCCAGCACCCTGCCCTGCCTGTGCATCCACGCTATGACACCCGAGAGG
GTGCAGCGGCTGAAGGCCAGCAGGCCCGAGCAGATCACCGTGTGCAGCTCCTTC
CCACTGCCCAAGTACAGGATCACCGGCGTGCAGAAGATCCAGTGCAGCCAGCCC
ATCCTGTTCAGCCCAAAGGTGCCCGCCTACATCCACCCCAGGAAGTACCTGGTGG
AGACCCCACCCGTGGACGAGACACCCGAGCCAAGCGCCGAGAACCAGAGCACC
GAGGGCACACCCGAGCAGCCACCCCTGATCACCGAGGACGAGACAAGGACCCG
GACCCCAGAGCCCATCATTATCGAGGAAGAGGAAGAGGACAGCATCAGCCTGCT
GAGCGACGGCCCCACCCACCAGGTGCTGCAGGTGGAGGCCGACATCCACGGCCC
ACCCAGCGTGTCCAGCTCCAGCTGGAGCATCCCACACGCCAGCGACTTCGACGT
GGACAGCCTGAGCATCCTGGACACCCTGGAGGGCGCCAGCGTGACCTCCGGCGC
CACCAGCGCCGAGACCAACAGCTACTTCGCCAAGAGCATGGAGTTCCTGGCCAG
GCCCGTGCCAGCTCCCAGGACCGTGTTCAGGAACCCACCCCACCCAGCTCCCAG
GACCAGGACCCCAAGCCTGGCTCCCAGCAGGGCCTGCAGCAGGACCAGCCTGGT
GAGCACCCCACCCGGCGTGAACAGGGTGATCACCAGGGAGGAACTGGAGGCCCT
GACACCCAGCAGGACCCCCAGCAGGTCCGTGAGCAGGACTAGTCTGGTGTCCAA
CCCACCCGGCGTGAACAGGGTGATCACCAGGGAGGAATTCGAGGCCTTCGTGGC
CCAGCAACAGAGACGGTTCGACGCCGGCGCCTACATCTTCAGCAGCGACACCGG
CCAGGGACACCTGCAGCAAAAGAGCGTGAGGCAGACCGTGCTGAGCGAGGTGG
TGCTGGAGAGGACCGAGCTGGAAATCAGCTACGCCCCCAGGCTGGACCAGGAGA
AGGAGGAACTGCTCAGGAAGAAACTGCAGCTGAACCCCACCCCAGCCAACAGG
AGCAGGTACCAGAGCAGGAAGGTGGAGAACATGAAGGCCATCACCGCCAGGCG
GATCCTGCAGGGCCTGGGACACTACCTGAAGGCCGAGGGCAAGGTGGAGTGCTA
CAGGACCCTGCACCCCGTGCCACTGTACAGCTCCAGCGTGAACAGGGCCTTCTCC
AGCCCCAAGGTGGCCGTGGAGGCCTGCAACGCTATGCTGAAGGAGAACTTCCCC
ACCGTGGCCAGCTACTGCATCATCCCCGAGTACGACGCCTACCTGGACATGGTGG
ACGGCGCCAGCTGCTGCCTGGACACCGCCAGCTTCTGCCCCGCCAAGCTGAGGA
GCTTCCCCAAGAAACACAGCTACCTGGAGCCCACCATCAGGAGCGCCGTGCCCA
GCGCCATCCAGAACACCCTGCAGAACGTGCTGGCCGCTGCCACCAAGAGGAACT
GCAACGTGACCCAGATGAGGGAGCTGCCCGTGCTGGACAGCGCTGCCTTCAACG
TGGAGTGCTTCAAGAAATACGCCTGCAACAACGAGTACTGGGAGACCTTCAAGG
AGAACCCCATCAGGCTGACCGAAGAGAACGTGGTGAACTACATCACCAAGCTGA
AGGGCCCCAAGGCCGCTGCCCTGTTCGCTAAGACCCACAACCTGAACATGCTGC
AGGACATCCCAATGGACAGGTTCGTGATGGACCTGAAGAGGGACGTGAAGGTGA
CACCCGGCACCAAGCACACCGAGGAGAGGCCCAAGGTGCAGGTGATCCAGGCC
GCTGACCCACTGGCCACCGCCTACCTGTGCGGCATCCACAGGGAGCTGGTGAGG
CGGCTGAACGCCGTGCTGCTGCCCAACATCCACACCCTGTTCGACATGAGCGCCG
AGGACTTCGACGCCATCATCGCCGAGCACTTCCAGCCCGGCGACTGCGTGCTGG
AGACCGACATCGCCAGCTTCGACAAGAGCGAGGATGACGCTATGGCCCTGACCG
CTCTGATGATCCTGGAGGACCTGGGCGTGGACGCCGAGCTGCTCACCCTGATCGA
GGCTGCCTTCGGCGAGATCAGCTCCATCCACCTGCCCACCAAGACCAAGTTCAAG
TTCGGCGCTATGATGAAAAGCGGAATGTTCCTGACCCTGTTCGTGAACACCGTGA
TCAACATTGTGATCGCCAGCAGGGTGCTGCGGGAGAGGCTGACCGGCAGCCCCT
GCGCTGCCTTCATCGGCGACGACAACATCGTGAAGGGCGTGAAAAGCGACAAGC
TGATGGCCGACAGGTGCGCCACCTGGCTGAACATGGAGGTGAAGATCATCGACG
CCGTGGTGGGCGAGAAGGCCCCCTACTTCTGCGGCGGATTCATCCTGTGCGACAG
CGTGACCGGCACCGCCTGCAGGGTGGCCGACCCCCTGAAGAGGCTGTTCAAGCT
GGGCAAGCCACTGGCCGCTGACGATGAGCACGACGATGACAGGCGGAGGGCCCT
GCACGAGGAAAGCACCAGGTGGAACAGGGTGGGCATCCTGAGCGAGCTGTGCA
AGGCCGTGGAGAGCAGGTACGAGACCGTGGGCACCAGCATCATCGTGATGGCTA
TGACCACACTGGCCAGCTCCGTCAAGAGCTTCTCCTACCTGAGGGGGGCCCCTAT
AACTCTCTACGGCTAACCTGAATGGACTACGACATAGTCTAGTCCGCCAAGGCCG
CCACCACTCGAGTATGTTACGTGCAAAGGTGATTGTCACCCCCCGAAAGACCATA
TTGTGACACACCCTCAGTATCACGCCCAAACATTTACAGCCGCGGTGTCAAAAAC
CGCGTGGACGTGGTTAACATCCCTGCTGGGAGGATCAGCCGTAATTATTATAATT
GGCTTGGTGCTGGCTACTATTGTGGCCATGTACGTGCTGACCAACCAGAAACATA
ATTGAATACAGCAGCAATTGGCAAGCTGCTTACATAGAACTCGCGGCGATTGGC
ATGCCGCCTTAAAATTTTTATTTTATTTTTTCTTTTCTTTTCCGAATCGGATTTTGT
TTTTAATATTTCAAAAAAAAAAAAAAAAAAAAAAAAATCTAGAAAAAAAAAAA
AAAAAAAAAAAAAAAAAAAAAAAAAAAAAAAAAAAAAAAAAAAAAAAAAAAA
AAAAAAAAAAAAAAAAAAAAAAAAAAAAAAAAAAAAA
SEQ ID NO: 79
MEKVHVDIEEDSPFLRALQRSFPQFEVEAKQVTDNDHANARAFSHLASKLIETEVDP
SDTILDIGSAPARRMYSKHKYHCICPMRCAEDPDRLYKYATKLKKNCKEITDKELDK
KMKELAAVMSDPDLETETMCLHDDESCRYEGQVAVYQDVYAVDGPTSLYHQANK
GVRVAYWIGFDTTPFMFKNLAGAYPSYSTNWADETVLTARNIGLCSSDVMERSRRG
MSILRKKYLKPSNNVLFSVGSTIYHEKRDLLRSWHLPSVFHLRGKQNYTCRCETIVSC
DGYVVKRIAISPGLYGKPSGYAATMHREGFLCCKVTDTLNGERVSFPVCTYVPATLC
DQMTGILATDVSADDAQKLLVGLNQRIVVNGRTQRNTNTMKNYLLPVVAQAFARW
AKEYKEDQEDERPLGLRDRQLVMGCCWAFRRHKITSIYKRPDTQTIIKVNSDFHSFV
LPRIGSNTLEIGLRTRIRKMLEEHKEPSPLITAEDVQEAKCAADEAKEVREAEELRAA
LPPLAADVEEPTLEADVDLMLQEAGAGSVETPRGLIKVTSYDGEDKIGSYAVLSPQA
VLKSEKLSCIHPLAEQVIVITHSGRKGRYAVEPYHGKVVVPEGHAIPVQDFQALSESA
TIVYNEREFVNRYLHHIATHGGALNTDEEYYKTVKPSEHDGEYLYDIDRKQCVKKEL
VTGLGLTGELVDPPFHEFAYESLRTRPAAPYQVPTIGVYGVPGSGKSGIIKSAVTKKD
LVVSAKKENCAEIIRDVKKMKGLDVNARTVDSVLLNGCKHPVETLYIDEAFACHAG
TLRALIAIIRPKKAVLCGDPKQCGFFNMMCLKVHFNHEICTQVFHKSISRRCTKSVTS
VVSTLFYDKKMRTTNPKETKIVIDTTGSTKPKQDDLILTCFRGWVKQLQIDYKGNEI
MTAAASQGLTRKGVYAVRYKVNENPLYAPTSEHVNVLLTRTEDRIVWKTLAGDPW
IKTLTAKYPGNFTATIEEWQAEHDAIMRHILERPDPTDVFQNKANVCWAKALVPVL
KTAGIDMTTEQWNTVDYFETDKAHSAEIVLNQLCVRFFGLDLDSGLFSAPTVPLSIR
NNHWDNSPSPNMYGLNKEVVRQLSRRYPQLPRAVATGRVYDMNTGTLRNYDPRIN
LVPVNRRLPHALVLHHNEHPQSDFSSFVSKLKGRTVLVVGEKLSVPGKMVDWLSDR
PEATFRARLDLGIPGDVPKYDIIFVNVRTPYKYHHYQQCEDHAIKLSMLTKKACLHL
NPGGTCVSIGYGYADRASESIIGAIARLFKFSRVCKPKSSLEETEVLFVFIGYDRKART
HNPYKLSSTLTNIYTGSRLHEAGCAPSYHVVRGDIATATEGVIINAANSKGQPGGGV
CGALYKKFPESFDLQPIEVGKARLVKGAAKHIIHAVGPNFNKVSEVEGDKQLAEAYE
SIAKIVNDNNYKSVAIPLLSTGIFSGNKDRLTQSLNHLLTALDTTDADVAIYCRDKKW
EMTLKEAVARREAVEEICISDDSSVTEPDAELVRVHPKSSLAGRKGYSTSDGKTFSYL
EGTKFHQAAKDIAEINAMWPVATEANEQVCMYILGESMSSIRSKCPVEESEASTPPST
LPCLCIHAMTPERVQRLKASRPEQITVCSSFPLPKYRITGVQKIQCSQPILFSPKVPAYI
HPRKYLVETPPVDETPEPSAENQSTEGTPEQPPLITEDETRTRTPEPIIIEEEEEDSISLLS
DGPTHQVLQVEADIHGPPSVSSSSWSIPHASDFDVDSLSILDTLEGASVTSGATSAETN
SYFAKSMEFLARPVPAPRTVFRNPPHPAPRTRTPSLAPSRACSRTSLVSTPPGVNRVIT
REELEALTPSRTPSRSVSRTSLVSNPPGVNRVITREEFEAFVAQQQRRFDAGAYIFSSD
TGQGHLQQKSVRQTVLSEVVLERTELEISYAPRLDQEKEELLRKKLQLNPTPANRSR
YQSRKVENMKAITARRILQGLGHYLKAEGKVECYRTLHPVPLYSSSVNRAFSSPKVA
VEACNAMLKENFPTVASYCIIPEYDAYLDMVDGASCCLDTASFCPAKLRSFPKKHSY
LEPTIRSAVPSAIQNTLQNVLAAATKRNCNVTQMRELPVLDSAAFNVECFKKYACNN
EYWETFKENPIRLTEENVVNYITKLKGPKAAALFAKTHNLNMLQDIPMDRFVMDLK
RDVKVTPGTKHTEERPKVQVIQAADPLATAYLCGIHRELVRRLNAVLLPNIHTLFDM
SAEDFDAIIAEHFQPGDCVLETDIASFDKSEDDAMALTALMILEDLGVDAELLTLIEA
AFGEISSIHLPTKTKFKFGAMMKSGMFLTLFVNTVINIVIASRVLRERLTGSPCAAFIG
DDNIVKGVKSDKLMADRCATWLNMEVKIIDAVVGEKAPYFCGGFILCDSVTGTACR
VADPLKRLFKLGKPLAADDEHDDDRRRALHEESTRWNRVGILSELCKAVESRYETV
GTSIIVMAMTTLASSVKSFSYLRGAPITLYG
SEQ ID NO: 80
MPEKVHVDIEEDSPFLRALQRSFPQFEVEAKQVTDNDHANARAFSHLASKLIETEVD
PSDTILDIGSAPARRMYSKHKYHCICPMRCAEDPDRLYKYATKLKKNCKEITDKELD
KKMKELAAVMSDPDLETETMCLHDDESCRYEGQVAVYQDVYAVDGPTSLYHQAN
KGVRVAYWIGFDTTPFMFKNLAGAYPSYSTNWADETVLTARNIGLCSSDVMERSRR
GMSILRKKYLKPSNNVLFSVGSTIYHEKRDLLRSWHLPSVFHLRGKQNYTCRCETIVS
CDGYVVKRIAISPGLYGKPSGYAATMHREGFLCCKVTDTLNGERVSFPVCTYVPATL
CDQMTGILATDVSADDAQKLLVGLNQRIVVNGRTQRNTNTMKNYLLPVVAQAFAR
WAKEYKEDQEDERPLGLRDRQLVMGCCWAFRRHKITSIYKRPDTQTIIKVNSDFHSF
VLPRIGSNTLEIGLRTRIRKMLEEHKEPSPLITAEDVQEAKCAADEAKEVREAEELRA
ALPPLAADVEEPTLEADVDLMLQEAGAGSVETPRGLIKVTSYDGEDKIGSYAVLSPQ
AVLKSEKLSCIHPLAEQVIVITHSGRKGRYAVEPYHGKVVVPEGHAIPVQDFQALSES
ATIVYNEREFVNRYLHHIATHGGALNTDEEYYKTVKPSEHDGEYLYDIDRKQCVKK
ELVTGLGLTGELVDPPFHEFAYESLRTRPAAPYQVPTIGVYGVPGSGKSGIIKSAVTK
KDLVVSAKKENCAEIIRDVKKMKGLDVNARTVDSVLLNGCKHPVETLYIDEAFACH
AGTLRALIAIIRPKKAVLCGDPKQCGFFNMMCLKVHFNHEICTQVFHKSISRRCTKSV
TSVVSTLFYDKKMRTTNPKETKIVIDTTGSTKPKQDDLILTCFRGWVKQLQIDYKGN
EIMTAAASQGLTRKGVYAVRYKVNENPLYAPTSEHVNVLLTRTEDRIVWKTLAGDP
WIKTLTAKYPGNFTATIEEWQAEHDAIMRHILERPDPTDVFQNKANVCWAKALVPV
LKTAGIDMTTEQWNTVDYFETDKAHSAEIVLNQLCVRFFGLDLDSGLFSAPTVPLSIR
NNHWDNSPSPNMYGLNKEVVRQLSRRYPQLPRAVATGRVYDMNTGTLRNYDPRIN
LVPVNRRLPHALVLHHNEHPQSDFSSFVSKLKGRTVLVVGEKLSVPGKMVDWLSDR
PEATFRARLDLGIPGDVPKYDIIFVNVRTPYKYHHYQQCEDHAIKLSMLTKKACLHL
NPGGTCVSIGYGYADRASESIIGAIARLFKFSRVCKPKSSLEETEVLFVFIGYDRKART
HNPYKLSSTLTNIYTGSRLHEAGCAPSYHVVRGDIATATEGVIINAANSKGQPGGGV
CGALYKKFPESFDLQPIEVGKARLVKGAAKHIIHAVGPNFNKVSEVEGDKQLAEAYE
SIAKIVNDNNYKSVAIPLLSTGIFSGNKDRLTQSLNHLLTALDTTDADVAIYCRDKKW
EMTLKEAVARREAVEEICISDDSSVTEPDAELVRVHPKSSLAGRKGYSTSDGKTFSYL
EGTKFHQAAKDIAEINAMWPVATEANEQVCMYILGESMSSIRSKCPVEESEASTPPST
LPCLCIHAMTPERVQRLKASRPEQITVCSSFPLPKYRITGVQKIQCSQPILFSPKVPAYI
HPRKYLVETPPVDETPEPSAENQSTEGTPEQPPLITEDETRTRTPEPIIIEEEEEDSISLLS
DGPTHQVLQVEADIHGPPSVSSSSWSIPHASDFDVDSLSILDTLEGASVTSGATSAETN
SYFAKSMEFLARPVPAPRTVFRNPPHPAPRTRTPSLAPSRACSRTSLVSTPPGVNRVIT
REELEALTPSRTPSRSVSRTSLVSNPPGVNRVITREEFEAFVAQQQRRFDAGAYIFSSD
TGQGHLQQKSVRQTVLSEVVLERTELEISYAPRLDQEKEELLRKKLQLNPTPANRSR
YQSRKVENMKAITARRILQGLGHYLKAEGKVECYRTLHPVPLYSSSVNRAFSSPKVA
VEACNAMLKENFPTVASYCIIPEYDAYLDMVDGASCCLDTASFCPAKLRSFPKKHSY
LEPTIRSAVPSAIQNTLQNVLAAATKRNCNVTQMRELPVLDSAAFNVECFKKYACNN
EYWETFKENPIRLTEENVVNYITKLKGPKAAALFAKTHNLNMLQDIPMDRFVMDLK
RDVKVTPGTKHTEERPKVQVIQAADPLATAYLCGIHRELVRRLNAVLLPNIHTLFDM
SAEDFDAIIAEHFQPGDCVLETDIASFDKSEDDAMALTALMILEDLGVDAELLTLIEA
AFGEISSIHLPTKTKFKFGAMMKSGMFLTLFVNTVINIVIASRVLRERLTGSPCAAFIG
DDNIVKGVKSDKLMADRCATWLNMEVKIIDAVVGEKAPYFCGGFILCDSVTGTACR
VADPLKRLFKLGKPLAADDEHDDDRRRALHEESTRWNRVGILSELCKAVESRYETV
GTSIIVMAMTTLASSVKSFSYLRGAPITLYG
SEQ ID NO: 81
MEKVHVDIEEDSPFLRALQRSFPQFEVEAKQVTDNDHANARAFSHLASKLIETEVDP
SDTILDIGSAPARRMYSKHKYHCICPMRCAEDPDRLYKYATKLKKNCKEITDKELDK
KMKELAAVMSDPDLETETMCLHDDESCRYEGQVAVYQDVYAVDGPTSLYHQANK
GVRVAYWIGFDTTPFMFKNLAGAYPSYSTNWADETVLTARNIGLCSSDVMERSRRG
MSILRKKYLKPSNNVLFSVGSTIYHEKRDLLRSWHLPSVFHLRGKQNYTCRCETIVSC
DGYVVKRIAISPGLYGKPSGYAATMHREGFLCCKVTDTLNGERVSFPVCTYVPATLC
DQMTGILATDVSADDAQKLLVGLNQRIVVNGRTQRNTNTMKNYLLPVVAQAFARW
AKEYKEDQEDERPLGLRDRQLVMGCCWAFRRHKITSIYKRPDTQTIIKVNSDFHSFV
LPRIGSNTLEIGLRTRIRKMLEEHKEPSPLITAEDIQEAKCAADEAKEVREAEELRAAL
PPLAADFEEPTLEADVDLMLQEAGAGSVETPRGLIKVTSYAGEDKIGSYAVLSPQAV
LKSEKLSCIHPLAEQVIVITHSGRKGRYAVEPYHGKVVVPEGHAIPVQDFQALSESAT
IVYNEREFVNRYLHHIATHGGALNTDEEYYKTVKPSEHDGEYLYDIDRKQCVKKEL
VTGLGLTGELVDPPFHEFAYESLRTRPAAPYQVPTIGVYGVPGSGKSGIIKSAVTKKD
LVVSAKKENCAEIIRDVKKMKGLDVNARTVDSVLLNGCKHPVETLYIDEAFACHAG
TLRALIAIIRPKKAVLCGDPKQCGFFNMMCLKVHFNHEICTQVFHKSISRRCTKSVTS
VVSTLFYDKRMRTTNPKETKIVIDTTGSTKPKQDDLILTCFRGWVKQLQIDYKGNEI
MTAAASQGLTRKGVYAVRYKVNENPLYAPTSEHVNVLLTRTEDRIVWKTLAGDPW
IKILTAKYPGNFTATIEEWQAEHDAIMRHILERPDPTDVFQNKANVCWAKALVPVLK
TAGIDMTTEQWNTVDYFETDKAHSAEIVLNQLCVRFFGLDLDSGLFSAPTVPLSIRN
NHWDNSPSPNMYGLNKEVVRQLSRRYPQLPRAVATGRVYDMNTGTLRNYDPRINL
VPVNRRLPHALVLHHNEHPQSDFSSFVSKLKGRTVLVVGEKLSVPGKKVDWLSDQP
EATFRARLDLGIPGDVPKYDIVFINVRTPYKYHHYQQCEDHAIKLSMLTKKACLHLN
PGGTCVSIGYGYADRASESIIGAIARQFKFSRVCKPKSSHEETEVLFVFIGYDRKARTH
NPYKLSSTLTNIYTGSRLHEAGCAPSYHVVRGDIATATEGVIINAANSKGQPGGGVC
GALYKKFPESFDLQPIEVGKARLVKGAAKHIIHAVGPNFNKVSEVEGDKQLAEAYES
IAKIVNDNNYKSVAIPLLSTGIFSGNKDRLTQSLNHLLTALDTTDADVAIYCRDKKWE
MTLKEAVARREAVEEICISDDSSVTEPDAELVRVHPKSSLAGRKGYSTSDGKTFSYLE
GTKFHQAAKDIAEINAMWPVATEANEQVCMYILGESMSSIRSKCPVEESEASTPPSTL
PCLCIHAMTPERVQRLKASRPEQITVCSSFPLPKYRITGVQKIQCSQPILFSPKVPAYIH
PRKYLVETPPVEETPESPAENQSTEGTPEQPALVNVDATRTRMPEPIIIEEEEEDSISLL
SDGPTHQVLQVEADIHGSPSVSSSSWSIPHASDFDVDSLSILDTLDGASVTSGAVSAET
NSYFARSMEFRARPVPAPRTVFRNPPHPAPRTRTPPLAHSRASSRTSLVSTPPGVNRVI
TREELEALTPSRAPSRSASRTSLVSNPPGVNRVITREEFEAFVAQQQ*RFDAGAYIFSS
DTGQGHLQQKSVRQTVLSEVVLERTELEISYAPRLDQEKEELLRKKLQLNPTPANRS
RYQSRRVENMKAITARRILQGLGHYLKAEGKVECYRTLHPVPLYSSSVNRAFSSPKV
AVEACNAMLKENFPTVASYCIIPEYDAYLDMVDGASCCLDTASFCPAKLRSFPKKHS
YLEPTIRSAVPSAIQNTLQNVLAAATKRNCNVTQMRELPVLDSAAFNVECFKKYACN
NEYWETFKENPIRLTEENVVNYITKLKGPKAAALFAKTHNLNMLQDIPMDRFVMDL
KRDVKVTPGTKHTEERPKVQVIQAADPLATADLCGIHRELVRRLNAVLLPNIHTLFD
MSAEDFDAIIAEHFQPGDCVLETDIASFDKSEDDAMALTALMILEDLGVDAELLTLIE
AAFGEISSIHLPTKTKFKFGAMMKSGMFLTLFVNTVINIVIASRVLRERLTGSPCAAFI
GDDNIVKGVKSDKLMADRCATWLNMEVKIIDAVVGEKAPYFCGGFILCDSVTGTAC
RVADPLKRLFKLGKPLAVDDEHDDDRRRALHEESTRWNRVGILPELCKAVESRYET
VGTSIIVMAMTTLASSVKSFSYLRGAPITLYG*
SEQ ID NO: 82
AGGAAACTTAAGTCAACACAACATATACAAAACAAACGAATCTCAAGCAATCAA
GCATTCTACTTCTATTGCAGCAATTTAAATCATTTCTTTTAAAGCAAAAGCAATTT
TCTGAAAATTTTCACCATTTACGAACGATAGCCACC
SEQ ID NO: 83
ACTCGAGCTAGTGACTGACTAGGATCTGGTTACCACTAAACCAGCCTCAAGAAC
ACCCGAATGGAGTCTCTAAGCTACATAATACCAACTTACACTTACAAAATGTTGT
CCCCCAAAATGTAGCCATTCGTATCTGCTCCTAATAAAAAGAAAGTTTCTTCACA
TTCTAGAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAA
AA
SEQ ID NO: 1
GAGGAAACTT AAGAUGGG
SEQ ID NO: 2
GGAUGGG
SEQ ID NO: 3
GGAUAGG
SEQ ID NO: 4
GGAGAGG
SEQ ID NO: 58
GGGAUGGG
SEQ ID NO: 59
GAGAGG
SEQ ID NO: 60
GAGGG
SEQ ID NO: 61
GAGAUGGG
SEQ ID NO: 62
GAGUGG
SEQ ID NO: 63
GAGGGG
SEQ ID NO: 64
GAGUAGG
SEQ ID NO: 65
GAGUGGG
SEQ ID NO: 66
GAUGGG
(RNA sequence for a construct with two subgenomic promoters, Luc, and E3L)
SEQ ID NO: 117
atgggcggcgcatgagagaagcccagaccaattacctacccaaaatggagaaagttcacgttgacatcgaggaagacagcccatt
cctcagagctttgcagcggagcttcccgcagtttgaggtagaagccaagcaggtcactgataatgaccatgctaatgccagagcgtt
ttcgcatctggcttcaaaactgatcgaaacggaggtggacccatccgacacgatccttgacattggaagtgcgcccgcccgcagaat
gtattctaagcacaagtatcattgtatctgtccgatgagatgtgcggaagatccggacagattgtataagtatgcaactaagctgaa
gaaaaactgtaaggaaataactgataaggaattggacaagaaaatgaaggagctggccgccgtcatgagcgaccctgacctgga
aactgagactatgtgcctccacgacgacgagtcgtgtcgctacgaagggcaagtcgctgtttaccaggatgtatacgcCGTCGAC
GGCCCCACCAGCCTGTACCACCAGGCCAACAAGGGCGTGAGGGTGGCCTACTGGATCGGCTTCGAC
ACCACACCCTTCATGTTCAAGAACCTGGCCGGCGCCTACCCCAGCTACAGCACCAACTGGGCCGACG
AGACCGTGCTGACCGCCAGGAACATCGGCCTGTGCAGCAGCGACGTGATGGAGAGGAGCCGGAGA
GGCATGAGCATCCTGAGGAAGAAATACCTGAAGCCCAGCAACAACGTGCTGTTCAGCGTGGGCAGC
ACCATCTACCACGAGAAGAGGGACCTGCTCAGGAGCTGGCACCTGCCCAGCGTGTTCCACCTGAGG
GGCAAGCAGAACTACACCTGCAGGTGCGAGACCATCGTGAGCTGCGACGGCTACGTGGTGAAGAG
GATCGCCATCAGCCCCGGCCTGTACGGCAAGCCCAGCGGCTACGCCGCTACAATGCACAGGGAGGG
CTTCCTGTGCTGCAAGGTGACCGACACCCTGAACGGCGAGAGGGTGAGCTTCCCCGTGTGCACCTA
CGTGCCCGCCACCCTGTGCGACCAGATGACCGGCATCCTGGCCACCGACGTGAGCGCCGACGACGC
CCAGAAGCTGCTCGTGGGCCTGAACCAGAGGATCGTGGTCAACGGCAGGACCCAGAGGAACACCA
ACACAATGAAGAACTACCTGCTGCCCGTGGTGGCCCAGGCTTTCGCCAGGTGGGCCAAGGAGTACA
AGGAGGACCAGGAAGACGAGAGGCCCCTGGGCCTGAGGGACAGGCAGCTGGTGATGGGCTGCTG
CTGGGCCTTCAGGCGGCACAAGATCACCAGCATCTACAAGAGGCCCGACACCCAGACCATCATCAA
GGTGAACAGCGACTTCCACAGCTTCGTGCTGCCCAGGATCGGCAGCAACACCCTGGAGATCGGCCT
GAGGACCCGGATCAGGAAGATGCTGGAGGAACACAAGGAGCCCAGCCCACTGATCACCGCCGAGG
ACGTGCAGGAGGCCAAGTGCGCTGCCGACGAGGCCAAGGAGGTGAGGGAGGCCGAGGAACTGAG
GGCCGCCCTGCCACCCCTGGCTGCCGACGTGGAGGAACCCACCCTGGAAGCCGACGTGGACCTGAT
GCTGCAGGAGGCCGGCGCCGGAAGCGTGGAGACACCCAGGGGCCTGATCAAGGTGACCAGCTACG
ACGGCGAGGACAAGATCGGCAGCTACGCCGTGCTGAGCCCACAGGCCGTGCTGAAGTCCGAGAAG
CTGAGCTGCATCCACCCACTGGCCGAGCAGGTGATCGTGATCACCCACAGCGGCAGGAAGGGCAG
GTACGCCGTGGAGCCCTACCACGGCAAGGTGGTCGTGCCCGAGGGCCACGCCATCCCCGTGCAGGA
CTTCCAGGCCCTGAGCGAGAGCGCCACCATCGTGTACAACGAGAGGGAGTTCGTGAACAGGTACCT
GCACCATATCGCCACCCACGGCGGAGCCCTGAACACCGACGAGGAATACTACAAGACCGTGAAGCC
CAGCGAGCACGACGGCGAGTACCTGTACGACATCGACAGGAAGCAGTGCGTGAAGAAAGAGCTGG
TGACCGGCCTGGGACTGACCGGCGAGCTGGTGGACCCACCCTTCCACGAGTTCGCCTACGAGAGCC
TGAGGACCAGACCCGCCGCTCCCTACCAGGTGCCCACCATCGGCGTGTACGGCGTGCCCGGCAGCG
GAAAGAGCGGCATCATCAAGAGCGCCGTGACCAAGAAAGACCTGGTGGTCAGCGCCAAGAAAGAG
AACTGCGCCGAGATCATCAGGGACGTGAAGAAGATGAAAGGCCTGGACGTGAACGCGCGCACCGT
GGACAGCGTGCTGCTGAACGGCTGCAAGCACCCCGTGGAGACCCTGTACATCGACGAGGCCTTCGC
TTGCCACGCCGGCACCCTGAGGGCCCTGATCGCCATCATCAGGCCCAAGAAAGCCGTGCTGTGCGG
CGACCCCAAGCAGTGCGGCTTCTTCAACATGATGTGCCTGAAGGTGCACTTCAACCACGAGATCTGC
ACCCAGGTGTTCCACAAGAGCATCAGCAGGCGGTGCACCAAGAGCGTGACCAGCGTCGTGAGCACC
CTGTTCTACGACAAGAAAATGAGGACCACCAACCCCAAGGAGACCAAAATCGTGATCGACACCACA
GGCAGCACCAAGCCCAAGCAGGACGACCTGATCCTGACCTGCTTCAGGGGCTGGGTGAAGCAGCTG
CAGATCGACTACAAGGGCAACGAGATCATGACCGCCGCTGCCAGCCAGGGCCTGACCAGGAAGGG
CGTGTACGCCGTGAGGTACAAGGTGAACGAGAACCCACTGTACGCTCCCACCAGCGAGCACGTGAA
CGTGCTGCTGACCAGGACCGAGGACAGGATCGTGTGGAAGACCCTGGCCGGCGACCCCTGGATCA
AGACCCTGACCGCCAAGTACCCCGGCAACTTCACCGCCACCATCGAAGAGTGGCAGGCCGAGCACG
ACGCCATCATGAGGCACATCCTGGAGAGGCCCGACCCCACCGACGTGTTCCAGAACAAGGCCAACG
TGTGCTGGGCCAAGGCCCTGGTGCCCGTGCTGAAGACCGCCGGCATCGACATGACCACAGAGCAGT
GGAACACCGTGGACTACTTCGAGACCGACAAGGCCCACAGCGCCGAGATCGTGCTGAACCAGCTGT
GCGTGAGGTTCTTCGGCCTGGACCTGGACAGCGGCCTGTTCAGCGCCCCCACCGTGCCACTGAGCAT
CAGGAACAACCACTGGGACAACAGCCCCAGCCCAAACATGTACGGCCTGAACAAGGAGGTGGTCA
GGCAGCTGAGCAGGCGGTACCCACAGCTGCCCAGGGCCGTGGCCACCGGCAGGGTGTACGACATG
AACACCGGCACCCTGAGGAACTACGACCCCAGGATCAACCTGGTGCCCGTGAACAGGCGGCTGCCC
CACGCCCTGGTGCTGCACCACAACGAGCACCCACAGAGCGACTTCAGCTCCTTCGTGAGCAAGCTGA
AAGGCAGGACCGTGCTGGTCGTGGGCGAGAAGCTGAGCGTGCCCGGCAAGATGGTGGACTGGCTG
AGCGACAGGCCCGAGGCCACCTTCCGGGCCAGGCTGGACCTCGGCATCCCCGGCGACGTGCCCAAG
TACGACATCATCTTCGTGAACGTCAGGACCCCATACAAGTACCACCATTACCAGCAGTGCGAGGACC
ACGCCATCAAGCTGAGCATGCTGACCAAGAAGGCCTGCCTGCACCTGAACCCCGGAGGCACCTGCG
TGAGCATCGGCTACGGCTACGCCGACAGGGCCAGCGAGAGCATCATTGGCGCCATCGCCAGGCTGT
TCAAGTTCAGCAGGGTGTGCAAACCCAAGAGCAGCCTGGAGGAAACCGAGGTGCTGTTCGTGTTCA
TCGGCTACGACCGGAAGGCCAGGACCCACAACCCCTACAAGCTGAGCAGCACCCTGACAAACATCT
ACACCGGCAGCAGGCTGCACGAGGCCGGCTGCGCCCCCAGCTACCACGTGGTCAGGGGCGATATC
GCCACCGCCACCGAGGGCGTGATCATCAACGCTGCCAACAGCAAGGGCCAGCCCGGAGGCGGAGT
GTGCGGCGCCCTGTACAAGAAGTTCCCCGAGAGCTTCGACCTGCAGCCCATCGAGGTGGGCAAGGC
CAGGCTGGTGAAGGGCGCCGCTAAGCACATCATCCACGCCGTGGGCCCCAACTTCAACAAGGTGAG
CGAGGTGGAAGGCGACAAGCAGCTGGCCGAAGCCTACGAGAGCATCGCCAAGATCGTGAACGACA
ATAACTACAAGAGCGTGGCCATCCCACTGCTCAGCACCGGCATCTTCAGCGGCAACAAGGACAGGC
TGACCCAGAGCCTGAACCACCTGCTCACCGCCCTGGACACCACCGATGCCGACGTGGCCATCTACTG
CAGGGACAAGAAGTGGGAGATGACCCTGAAGGAGGCCGTGGCCAGGCGGGAGGCCGTGGAAGAG
ATCTGCATCAGCGACGACTCCAGCGTGACCGAGCCCGACGCCGAGCTGGTGAGGGTGCACCCCAAG
AGCTCCCTGGCCGGCAGGAAGGGCTACAGCACCAGCGACGGCAAGACCTTCAGCTACCTGGAGGG
CACCAAGTTCCACCAGGCCGCTAAGGACATCGCCGAGATCAACGCTATGTGGCCCGTGGCCACCGA
GGCCAACGAGCAGGTGTGCATGTACATCCTGGGCGAGAGCATGTCCAGCATCAGGAGCAAGTGCCC
CGTGGAGGAAAGCGAGGCCAGCACACCACCCAGCACCCTGCCCTGCCTGTGCATCCACGCTATGAC
ACCCGAGAGGGTGCAGCGGCTGAAGGCCAGCAGGCCCGAGCAGATCACCGTGTGCAGCTCCTTCCC
ACTGCCCAAGTACAGGATCACCGGCGTGCAGAAGATCCAGTGCAGCCAGCCCATCCTGTTCAGCCCA
AAGGTGCCCGCCTACATCCACCCCAGGAAGTACCTGGTGGAGACCCCACCCGTGGACGAGACACCC
GAGCCAAGCGCCGAGAACCAGAGCACCGAGGGCACACCCGAGCAGCCACCCCTGATCACCGAGGA
CGAGACAAGGACCCGGACCCCAGAGCCCATCATTATCGAGGAAGAGGAAGAGGACAGCATCAGCC
TGCTGAGCGACGGCCCCACCCACCAGGTGCTGCAGGTGGAGGCCGACATCCACGGCCCACCCAGCG
TGTCCAGCTCCAGCTGGAGCATCCCACACGCCAGCGACTTCGACGTGGACAGCCTGAGCATCCTGG
ACACCCTGGAGGGCGCCAGCGTGACCTCCGGCGCCACCAGCGCCGAGACCAACAGCTACTTCGCCA
AGAGCATGGAGTTCCTGGCCAGGCCCGTGCCAGCTCCCAGGACCGTGTTCAGGAACCCACCCCACC
CAGCTCCCAGGACCAGGACCCCAAGCCTGGCTCCCAGCAGGGCCTGCAGCAGGACCAGCCTGGTGA
GCACCCCACCCGGCGTGAACAGGGTGATCACCAGGGAGGAACTGGAGGCCCTGACACCCAGCAGG
ACCCCCAGCAGGTCCGTGAGCAGGACTAGTCTGGTGTCCAACCCACCCGGCGTGAACAGGGTGATC
ACCAGGGAGGAATTCGAGGCCTTCGTGGCCCAGCAACAGAGACGGTTCGACGCCGGCGCCTACATC
TTCAGCAGCGACACCGGCCAGGGACACCTGCAGCAAAAGAGCGTGAGGCAGACCGTGCTGAGCGA
GGTGGTGCTGGAGAGGACCGAGCTGGAAATCAGCTACGCCCCCAGGCTGGACCAGGAGAAGGAG
GAACTGCTCAGGAAGAAACTGCAGCTGAACCCCACCCCAGCCAACAGGAGCAGGTACCAGAGCAG
GAAGGTGGAGAACATGAAGGCCATCACCGCCAGGCGGATCCTGCAGGGCCTGGGACACTACCTGA
AGGCCGAGGGCAAGGTGGAGTGCTACAGGACCCTGCACCCCGTGCCACTGTACAGCTCCAGCGTGA
ACAGGGCCTTCTCCAGCCCCAAGGTGGCCGTGGAGGCCTGCAACGCTATGCTGAAGGAGAACTTCC
CCACCGTGGCCAGCTACTGCATCATCCCCGAGTACGACGCCTACCTGGACATGGTGGACGGCGCCA
GCTGCTGCCTGGACACCGCCAGCTTCTGCCCCGCCAAGCTGAGGAGCTTCCCCAAGAAACACAGCTA
CCTGGAGCCCACCATCAGGAGCGCCGTGCCCAGCGCCATCCAGAACACCCTGCAGAACGTGCTGGC
CGCTGCCACCAAGAGGAACTGCAACGTGACCCAGATGAGGGAGCTGCCCGTGCTGGACAGCGCTG
CCTTCAACGTGGAGTGCTTCAAGAAATACGCCTGCAACAACGAGTACTGGGAGACCTTCAAGGAGA
ACCCCATCAGGCTGACCGAAGAGAACGTGGTGAACTACATCACCAAGCTGAAGGGCCCCAAGGCCG
CTGCCCTGTTCGCTAAGACCCACAACCTGAACATGCTGCAGGACATCCCAATGGACAGGTTCGTGAT
GGACCTGAAGAGGGACGTGAAGGTGACACCCGGCACCAAGCACACCGAGGAGAGGCCCAAGGTG
CAGGTGATCCAGGCCGCTGACCCACTGGCCACCGCCTACCTGTGCGGCATCCACAGGGAGCTGGTG
AGGCGGCTGAACGCCGTGCTGCTGCCCAACATCCACACCCTGTTCGACATGAGCGCCGAGGACTTC
GACGCCATCATCGCCGAGCACTTCCAGCCCGGCGACTGCGTGCTGGAGACCGACATCGCCAGCTTC
GACAAGAGCGAGGATGACGCTATGGCCCTGACCGCTCTGATGATCCTGGAGGACCTGGGCGTGGA
CGCCGAGCTGCTCACCCTGATCGAGGCTGCCTTCGGCGAGATCAGCTCCATCCACCTGCCCACCAAG
ACCAAGTTCAAGTTCGGCGCTATGATGAAAAGCGGAATGTTCCTGACCCTGTTCGTGAACACCGTGA
TCAACATTGTGATCGCCAGCAGGGTGCTGCGGGAGAGGCTGACCGGCAGCCCCTGCGCTGCCTTCA
TCGGCGACGACAACATCGTGAAGGGCGTGAAAAGCGACAAGCTGATGGCCGACAGGTGCGCCACC
TGGCTGAACATGGAGGTGAAGATCATCGACGCCGTGGTGGGCGAGAAGGCCCCCTACTTCTGCGGC
GGATTCATCCTGTGCGACAGCGTGACCGGCACCGCCTGCAGGGTGGCCGACCCCCTGAAGAGGCTG
TTCAAGCTGGGCAAGCCACTGGCCGCTGACGATGAGCACGACGATGACAGGCGGAGGGCCCTGCA
CGAGGAAAGCACCAGGTGGAACAGGGTGGGCATCCTGAGCGAGCTGTGCAAGGCCGTGGAGAGC
AGGTACGAGACCGTGGGCACCAGCATCATCGTGATGGCTATGACCACACTGGCCAGCTCCGTCAAG
AGCTTCTCCTACCTGAGGGGGGCCCCTATAACTCTCTACGGCTAACCTGAATGGACTACGACATAGT
CTAgccaccATGagcaagatctacatcgacgagcggagcaacgccgagatcgtgtgcgaggccatcaagaccatcggcatcga
gggcgccaccgccgcccagctgaccaggcagctgaacatggagaagcgggaggtgaacaaggccctgtacgacctgcagaggag
cgctatggtgtactccagcgacgacatccctccccggtggttcatgaccaccgaggccgacaagcccgacgccgacgctatggccg
acgtgatcatcgacgacgtgagcagggagaagtccatgagggaggaccacaagagcttcgacgacgtgatccccgccaagaaga
tcatcgactggaagggcgccaaccccgtgaccgtgatcaacgagtactgccagatcaccaggagggactggagcttccggatcga
gagcgtgggccccagcaacagccccaccttctacgcctgcgtggacatcgacggcagggtgttcgacaaggccgacggcaagagc
aagcgggacgccaagaacaacgccgccaagctggccgtggacaagctgctgggctacgtgatcatccggttcTAAactcgagcta
gtgactgactaggatctggttaccactaaaccagcctcaagaacacccgaatggagtctctaagctacataataccaacttacactt
acaaaatgttgtcccccaaaatgtagccattcgtatctgctcctaataaaaagaaagtttcttcacattctagAGCTCCGTCAAG
AGCTTCTCCTACCTGAGGGGGGCCCCTATAACTCTCTACGGCTAACCTGAATGGACTACGACATAGT
CTAGCCACCATGGAAGATGCCAAAAACATTAAGAAGGGCCCAGCGCCATTCTACCCACTCGAAGAC
GGGACCGCCGGCGAGCAGCTGCACAAAGCCATGAAGCGCTACGCCCTGGTGCCCGGCACCATCGCC
TTTACCGACGCACATATCGAGGTGGACATTACCTACGCCGAGTACTTCGAGATGAGCGTTCGGCTGG
CAGAAGCTATGAAGCGCTATGGGCTGAATACAAACCATCGGATCGTGGTGTGCAGCGAGAATAGCT
TGCAGTTCTTCATGCCCGTGTTGGGTGCCCTGTTCATCGGTGTGGCTGTGGCCCCAGCTAACGACAT
CTACAACGAGCGCGAGCTGCTGAACAGCATGGGCATCAGCCAGCCCACCGTCGTATTCGTGAGCAA
GAAAGGGCTGCAAAAGATCCTCAACGTGCAAAAGAAGCTACCGATCATACAAAAGATCATCATCAT
GGATAGCAAGACCGACTACCAGGGCTTCCAAAGCATGTACACCTTCGTGACTTCCCATTTGCCACCC
GGCTTCAACGAGTACGACTTCGTGCCCGAGAGCTTCGACCGGGACAAAACCATCGCCCTGATCATGA
ACAGTAGTGGCAGTACCGGATTGCCCAAGGGCGTAGCCCTACCGCACCGCACCGCTTGTGTCCGATT
CAGTCATGCCCGCGACCCCATCTTCGGCAACCAGATCATCCCCGACACCGCTATCCTCAGCGTGGTG
CCATTTCACCACGGCTTCGGCATGTTCACCACGCTGGGCTACTTGATCTGCGGCTTTCGGGTCGTGCT
CATGTACCGCTTCGAGGAGGAGCTATTCTTGCGCAGCTTGCAAGACTATAAGATTCAATCTGCCCTG
CTGGTGCCCACACTATTTAGCTTCTTCGCTAAGAGCACTCTCATCGACAAGTACGACCTAAGCAACTT
GCACGAGATCGCCAGCGGCGGGGCGCCGCTCAGCAAGGAGGTAGGTGAGGCCGTGGCCAAACGC
TTCCACCTACCAGGCATCCGACAGGGCTACGGCCTGACAGAAACAACCAGCGCCATTCTGATCACCC
CCGAAGGGGACGACAAGCCTGGCGCAGTAGGCAAGGTGGTGCCCTTCTTCGAGGCTAAGGTGGTG
GACTTGGACACCGGTAAGACACTGGGTGTGAACCAGCGCGGCGAGCTGTGCGTCCGTGGCCCCATG
ATCATGAGCGGCTACGTTAACAACCCCGAGGCTACAAACGCTCTCATCGACAAGGACGGCTGGCTG
CACAGCGGCGACATCGCCTACTGGGACGAGGACGAGCACTTCTTCATCGTGGACCGGCTGAAGTCC
CTGATCAAATACAAGGGCTACCAGGTAGCCCCAGCCGAACTGGAGAGCATCCTGCTGCAACACCCC
AACATCTTCGACGCCGGGGTCGCCGGCCTGCCCGACGACGATGCCGGCGAGCTGCCCGCCGCAGTC
GTCGTGCTGGAACACGGTAAAACCATGACCGAGAAGGAGATCGTGGACTATGTGGCCAGCCAGGTT
ACAACCGCCAAGAAGCTGCGCGGTGGTGTTGTGTTCGTGGACGAGGTGCCTAAAGGACTGACCGG
CAAGTTGGACGCCCGCAAGATCCGCGAGATTCTCATTAAGGCCAAGAAGGGCGGCAAGATCGCCGT
GTAACTCGAGTATGTTACGTGCAAAGGTGATTGTCACCCCCCGAAAGACCATATTGTGACACACCCT
CAGTATCACGCCCAAACATTTACAGCCGCGGTGTCAAAAACCGCGTGGACGTGGTTAACATCCCTGC
TGGGAGGATCAGCCGTAATTATTATAATTGGCTTGGTGCTGGCTACTATTGTGGCCATGTACGTGCT
GACCAACCAGAAACATAATTGAATACAGCAGCAATTGGCAAGCTGCTTACATAGAACTCGCGGCGA
TTGGCATGCCGCCTTAAAATTTTTATTTTATTTTTTCTTTTCTTTTCCGAATCGGATTTTGTTTTTAATAT
TTCAAAAAAAAAAAAAAAAAAAAAAAAATctagAAAAAAAAAAAAAAAAAAAAAAAAAAAAAAAAA
AAAAAAAAAAAAAAAAAAAAAAAAAAAAAAAAAAAAAAAAAAAAAAAaaaaaaaaaaaaaaaaaaaa
(RNA sequence for STARR Fluc IRES-E3L)
SEQ ID NO: 118
AUGGGCGGCGCAUGAGAGAAGCCCAGACCAAUUACCUACCCAAAAUGGAGAAA
GUUCACGUUGACAUCGAGGAAGACAGCCCAUUCCUCAGAGCUUUGCAGCGGAG
CUUCCCGCAGUUUGAGGUAGAAGCCAAGCAGGUCACUGAUAAUGACCAUGCUA
AUGCCAGAGCGUUUUCGCAUCUGGCUUCAAAACUGAUCGAAACGGAGGUGGA
CCCAUCCGACACGAUCCUUGACAUUGGAAGUGCGCCCGCCCGCAGAAUGUAUU
CUAAGCACAAGUAUCAUUGUAUCUGUCCGAUGAGAUGUGCGGAAGAUCCGGA
CAGAUUGUAUAAGUAUGCAACUAAGCUGAAGAAAAACUGUAAGGAAAUAACU
GAUAAGGAAUUGGACAAGAAAAUGAAGGAGCUGGCCGCCGUCAUGAGCGACC
CUGACCUGGAAACUGAGACUAUGUGCCUCCACGACGACGAGUCGUGUCGCUAC
GAAGGGCAAGUCGCUGUUUACCAGGAUGUAUACGCCGUCGACGGCCCCACCAG
CCUGUACCACCAGGCCAACAAGGGCGUGAGGGUGGCCUACUGGAUCGGCUUCG
ACACCACACCCUUCAUGUUCAAGAACCUGGCCGGCGCCUACCCCAGCUACAGC
ACCAACUGGGCCGACGAGACCGUGCUGACCGCCAGGAACAUCGGCCUGUGCAG
CAGCGACGUGAUGGAGAGGAGCCGGAGAGGCAUGAGCAUCCUGAGGAAGAAA
UACCUGAAGCCCAGCAACAACGUGCUGUUCAGCGUGGGCAGCACCAUCUACCA
CGAGAAGAGGGACCUGCUCAGGAGCUGGCACCUGCCCAGCGUGUUCCACCUGA
GGGGCAAGCAGAACUACACCUGCAGGUGCGAGACCAUCGUGAGCUGCGACGGC
UACGUGGUGAAGAGGAUCGCCAUCAGCCCCGGCCUGUACGGCAAGCCCAGCGG
CUACGCCGCUACAAUGCACAGGGAGGGCUUCCUGUGCUGCAAGGUGACCGACA
CCCUGAACGGCGAGAGGGUGAGCUUCCCCGUGUGCACCUACGUGCCCGCCACC
CUGUGCGACCAGAUGACCGGCAUCCUGGCCACCGACGUGAGCGCCGACGACGC
CCAGAAGCUGCUCGUGGGCCUGAACCAGAGGAUCGUGGUCAACGGCAGGACCC
AGAGGAACACCAACACAAUGAAGAACUACCUGCUGCCCGUGGUGGCCCAGGCU
UUCGCCAGGUGGGCCAAGGAGUACAAGGAGGACCAGGAAGACGAGAGGCCCCU
GGGCCUGAGGGACAGGCAGCUGGUGAUGGGCUGCUGCUGGGCCUUCAGGCGGC
ACAAGAUCACCAGCAUCUACAAGAGGCCCGACACCCAGACCAUCAUCAAGGUG
AACAGCGACUUCCACAGCUUCGUGCUGCCCAGGAUCGGCAGCAACACCCUGGA
GAUCGGCCUGAGGACCCGGAUCAGGAAGAUGCUGGAGGAACACAAGGAGCCCA
GCCCACUGAUCACCGCCGAGGACGUGCAGGAGGCCAAGUGCGCUGCCGACGAG
GCCAAGGAGGUGAGGGAGGCCGAGGAACUGAGGGCCGCCCUGCCACCCCUGGC
UGCCGACGUGGAGGAACCCACCCUGGAAGCCGACGUGGACCUGAUGCUGCAGG
AGGCCGGCGCCGGAAGCGUGGAGACACCCAGGGGCCUGAUCAAGGUGACCAGC
UACGACGGCGAGGACAAGAUCGGCAGCUACGCCGUGCUGAGCCCACAGGCCGU
GCUGAAGUCCGAGAAGCUGAGCUGCAUCCACCCACUGGCCGAGCAGGUGAUCG
UGAUCACCCACAGCGGCAGGAAGGGCAGGUACGCCGUGGAGCCCUACCACGGC
AAGGUGGUCGUGCCCGAGGGCCACGCCAUCCCCGUGCAGGACUUCCAGGCCCU
GAGCGAGAGCGCCACCAUCGUGUACAACGAGAGGGAGUUCGUGAACAGGUACC
UGCACCAUAUCGCCACCCACGGCGGAGCCCUGAACACCGACGAGGAAUACUAC
AAGACCGUGAAGCCCAGCGAGCACGACGGCGAGUACCUGUACGACAUCGACAG
GAAGCAGUGCGUGAAGAAAGAGCUGGUGACCGGCCUGGGACUGACCGGCGAG
CUGGUGGACCCACCCUUCCACGAGUUCGCCUACGAGAGCCUGAGGACCAGACC
CGCCGCUCCCUACCAGGUGCCCACCAUCGGCGUGUACGGCGUGCCCGGCAGCG
GAAAGAGCGGCAUCAUCAAGAGCGCCGUGACCAAGAAAGACCUGGUGGUCAGC
GCCAAGAAAGAGAACUGCGCCGAGAUCAUCAGGGACGUGAAGAAGAUGAAAG
GCCUGGACGUGAACGCGCGCACCGUGGACAGCGUGCUGCUGAACGGCUGCAAG
CACCCCGUGGAGACCCUGUACAUCGACGAGGCCUUCGCUUGCCACGCCGGCAC
CCUGAGGGCCCUGAUCGCCAUCAUCAGGCCCAAGAAAGCCGUGCUGUGCGGCG
ACCCCAAGCAGUGCGGCUUCUUCAACAUGAUGUGCCUGAAGGUGCACUUCAAC
CACGAGAUCUGCACCCAGGUGUUCCACAAGAGCAUCAGCAGGCGGUGCACCAA
GAGCGUGACCAGCGUCGUGAGCACCCUGUUCUACGACAAGAAAAUGAGGACCA
CCAACCCCAAGGAGACCAAAAUCGUGAUCGACACCACAGGCAGCACCAAGCCC
AAGCAGGACGACCUGAUCCUGACCUGCUUCAGGGGCUGGGUGAAGCAGCUGCA
GAUCGACUACAAGGGCAACGAGAUCAUGACCGCCGCUGCCAGCCAGGGCCUGA
CCAGGAAGGGCGUGUACGCCGUGAGGUACAAGGUGAACGAGAACCCACUGUAC
GCUCCCACCAGCGAGCACGUGAACGUGCUGCUGACCAGGACCGAGGACAGGAU
CGUGUGGAAGACCCUGGCCGGCGACCCCUGGAUCAAGACCCUGACCGCCAAGU
ACCCCGGCAACUUCACCGCCACCAUCGAAGAGUGGCAGGCCGAGCACGACGCC
AUCAUGAGGCACAUCCUGGAGAGGCCCGACCCCACCGACGUGUUCCAGAACAA
GGCCAACGUGUGCUGGGCCAAGGCCCUGGUGCCCGUGCUGAAGACCGCCGGCA
UCGACAUGACCACAGAGCAGUGGAACACCGUGGACUACUUCGAGACCGACAAG
GCCCACAGCGCCGAGAUCGUGCUGAACCAGCUGUGCGUGAGGUUCUUCGGCCU
GGACCUGGACAGCGGCCUGUUCAGCGCCCCCACCGUGCCACUGAGCAUCAGGA
ACAACCACUGGGACAACAGCCCCAGCCCAAACAUGUACGGCCUGAACAAGGAG
GUGGUCAGGCAGCUGAGCAGGCGGUACCCACAGCUGCCCAGGGCCGUGGCCAC
CGGCAGGGUGUACGACAUGAACACCGGCACCCUGAGGAACUACGACCCCAGGA
UCAACCUGGUGCCCGUGAACAGGCGGCUGCCCCACGCCCUGGUGCUGCACCAC
AACGAGCACCCACAGAGCGACUUCAGCUCCUUCGUGAGCAAGCUGAAAGGCAG
GACCGUGCUGGUCGUGGGCGAGAAGCUGAGCGUGCCCGGCAAGAUGGUGGAC
UGGCUGAGCGACAGGCCCGAGGCCACCUUCCGGGCCAGGCUGGACCUCGGCAU
CCCCGGCGACGUGCCCAAGUACGACAUCAUCUUCGUGAACGUCAGGACCCCAU
ACAAGUACCACCAUUACCAGCAGUGCGAGGACCACGCCAUCAAGCUGAGCAUG
CUGACCAAGAAGGCCUGCCUGCACCUGAACCCCGGAGGCACCUGCGUGAGCAU
CGGCUACGGCUACGCCGACAGGGCCAGCGAGAGCAUCAUUGGCGCCAUCGCCA
GGCUGUUCAAGUUCAGCAGGGUGUGCAAACCCAAGAGCAGCCUGGAGGAAACC
GAGGUGCUGUUCGUGUUCAUCGGCUACGACCGGAAGGCCAGGACCCACAACCC
CUACAAGCUGAGCAGCACCCUGACAAACAUCUACACCGGCAGCAGGCUGCACG
AGGCCGGCUGCGCCCCCAGCUACCACGUGGUCAGGGGCGAUAUCGCCACCGCC
ACCGAGGGCGUGAUCAUCAACGCUGCCAACAGCAAGGGCCAGCCCGGAGGCGG
AGUGUGCGGCGCCCUGUACAAGAAGUUCCCCGAGAGCUUCGACCUGCAGCCCA
UCGAGGUGGGCAAGGCCAGGCUGGUGAAGGGCGCCGCUAAGCACAUCAUCCAC
GCCGUGGGCCCCAACUUCAACAAGGUGAGCGAGGUGGAAGGCGACAAGCAGCU
GGCCGAAGCCUACGAGAGCAUCGCCAAGAUCGUGAACGACAAUAACUACAAGA
GCGUGGCCAUCCCACUGCUCAGCACCGGCAUCUUCAGCGGCAACAAGGACAGG
CUGACCCAGAGCCUGAACCACCUGCUCACCGCCCUGGACACCACCGAUGCCGA
CGUGGCCAUCUACUGCAGGGACAAGAAGUGGGAGAUGACCCUGAAGGAGGCC
GUGGCCAGGCGGGAGGCCGUGGAAGAGAUCUGCAUCAGCGACGACUCCAGCGU
GACCGAGCCCGACGCCGAGCUGGUGAGGGUGCACCCCAAGAGCUCCCUGGCCG
GCAGGAAGGGCUACAGCACCAGCGACGGCAAGACCUUCAGCUACCUGGAGGGC
ACCAAGUUCCACCAGGCCGCUAAGGACAUCGCCGAGAUCAACGCUAUGUGGCC
CGUGGCCACCGAGGCCAACGAGCAGGUGUGCAUGUACAUCCUGGGCGAGAGCA
UGUCCAGCAUCAGGAGCAAGUGCCCCGUGGAGGAAAGCGAGGCCAGCACACCA
CCCAGCACCCUGCCCUGCCUGUGCAUCCACGCUAUGACACCCGAGAGGGUGCA
GCGGCUGAAGGCCAGCAGGCCCGAGCAGAUCACCGUGUGCAGCUCCUUCCCAC
UGCCCAAGUACAGGAUCACCGGCGUGCAGAAGAUCCAGUGCAGCCAGCCCAUC
CUGUUCAGCCCAAAGGUGCCCGCCUACAUCCACCCCAGGAAGUACCUGGUGGA
GACCCCACCCGUGGACGAGACACCCGAGCCAAGCGCCGAGAACCAGAGCACCG
AGGGCACACCCGAGCAGCCACCCCUGAUCACCGAGGACGAGACAAGGACCCGG
ACCCCAGAGCCCAUCAUUAUCGAGGAAGAGGAAGAGGACAGCAUCAGCCUGCU
GAGCGACGGCCCCACCCACCAGGUGCUGCAGGUGGAGGCCGACAUCCACGGCC
CACCCAGCGUGUCCAGCUCCAGCUGGAGCAUCCCACACGCCAGCGACUUCGAC
GUGGACAGCCUGAGCAUCCUGGACACCCUGGAGGGCGCCAGCGUGACCUCCGG
CGCCACCAGCGCCGAGACCAACAGCUACUUCGCCAAGAGCAUGGAGUUCCUGG
CCAGGCCCGUGCCAGCUCCCAGGACCGUGUUCAGGAACCCACCCCACCCAGCUC
CCAGGACCAGGACCCCAAGCCUGGCUCCCAGCAGGGCCUGCAGCAGGACCAGC
CUGGUGAGCACCCCACCCGGCGUGAACAGGGUGAUCACCAGGGAGGAACUGGA
GGCCCUGACACCCAGCAGGACCCCCAGCAGGUCCGUGAGCAGGACUAGUCUGG
UGUCCAACCCACCCGGCGUGAACAGGGUGAUCACCAGGGAGGAAUUCGAGGCC
UUCGUGGCCCAGCAACAGAGACGGUUCGACGCCGGCGCCUACAUCUUCAGCAG
CGACACCGGCCAGGGACACCUGCAGCAAAAGAGCGUGAGGCAGACCGUGCUGA
GCGAGGUGGUGCUGGAGAGGACCGAGCUGGAAAUCAGCUACGCCCCCAGGCUG
GACCAGGAGAAGGAGGAACUGCUCAGGAAGAAACUGCAGCUGAACCCCACCCC
AGCCAACAGGAGCAGGUACCAGAGCAGGAAGGUGGAGAACAUGAAGGCCAUC
ACCGCCAGGCGGAUCCUGCAGGGCCUGGGACACUACCUGAAGGCCGAGGGCAA
GGUGGAGUGCUACAGGACCCUGCACCCCGUGCCACUGUACAGCUCCAGCGUGA
ACAGGGCCUUCUCCAGCCCCAAGGUGGCCGUGGAGGCCUGCAACGCUAUGCUG
AAGGAGAACUUCCCCACCGUGGCCAGCUACUGCAUCAUCCCCGAGUACGACGC
CUACCUGGACAUGGUGGACGGCGCCAGCUGCUGCCUGGACACCGCCAGCUUCU
GCCCCGCCAAGCUGAGGAGCUUCCCCAAGAAACACAGCUACCUGGAGCCCACC
AUCAGGAGCGCCGUGCCCAGCGCCAUCCAGAACACCCUGCAGAACGUGCUGGC
CGCUGCCACCAAGAGGAACUGCAACGUGACCCAGAUGAGGGAGCUGCCCGUGC
UGGACAGCGCUGCCUUCAACGUGGAGUGCUUCAAGAAAUACGCCUGCAACAAC
GAGUACUGGGAGACCUUCAAGGAGAACCCCAUCAGGCUGACCGAAGAGAACGU
GGUGAACUACAUCACCAAGCUGAAGGGCCCCAAGGCCGCUGCCCUGUUCGCUA
AGACCCACAACCUGAACAUGCUGCAGGACAUCCCAAUGGACAGGUUCGUGAUG
GACCUGAAGAGGGACGUGAAGGUGACACCCGGCACCAAGCACACCGAGGAGAG
GCCCAAGGUGCAGGUGAUCCAGGCCGCUGACCCACUGGCCACCGCCUACCUGU
GCGGCAUCCACAGGGAGCUGGUGAGGCGGCUGAACGCCGUGCUGCUGCCCAAC
AUCCACACCCUGUUCGACAUGAGCGCCGAGGACUUCGACGCCAUCAUCGCCGA
GCACUUCCAGCCCGGCGACUGCGUGCUGGAGACCGACAUCGCCAGCUUCGACA
AGAGCGAGGAUGACGCUAUGGCCCUGACCGCUCUGAUGAUCCUGGAGGACCUG
GGCGUGGACGCCGAGCUGCUCACCCUGAUCGAGGCUGCCUUCGGCGAGAUCAG
CUCCAUCCACCUGCCCACCAAGACCAAGUUCAAGUUCGGCGCUAUGAUGAAAA
GCGGAAUGUUCCUGACCCUGUUCGUGAACACCGUGAUCAACAUUGUGAUCGCC
AGCAGGGUGCUGCGGGAGAGGCUGACCGGCAGCCCCUGCGCUGCCUUCAUCGG
CGACGACAACAUCGUGAAGGGCGUGAAAAGCGACAAGCUGAUGGCCGACAGG
UGCGCCACCUGGCUGAACAUGGAGGUGAAGAUCAUCGACGCCGUGGUGGGCGA
GAAGGCCCCCUACUUCUGCGGCGGAUUCAUCCUGUGCGACAGCGUGACCGGCA
CCGCCUGCAGGGUGGCCGACCCCCUGAAGAGGCUGUUCAAGCUGGGCAAGCCA
CUGGCCGCUGACGAUGAGCACGACGAUGACAGGCGGAGGGCCCUGCACGAGGA
AAGCACCAGGUGGAACAGGGUGGGCAUCCUGAGCGAGCUGUGCAAGGCCGUG
GAGAGCAGGUACGAGACCGUGGGCACCAGCAUCAUCGUGAUGGCUAUGACCAC
ACUGGCCAGCUCCGUCAAGAGCUUCUCCUACCUGAGGGGGGCCCCUAUAACUC
UCUACGGCUAACCUGAAUGGACUACGACAUAGUCUAGUCCGCCAAGGCCGCCA
CCAUGGAAGAUGCCAAAAACAUUAAGAAGGGCCCAGCGCCAUUCUACCCACUC
GAAGACGGGACCGCCGGCGAGCAGCUGCACAAAGCCAUGAAGCGCUACGCCCU
GGUGCCCGGCACCAUCGCCUUUACCGACGCACAUAUCGAGGUGGACAUUACCU
ACGCCGAGUACUUCGAGAUGAGCGUUCGGCUGGCAGAAGCUAUGAAGCGCUA
UGGGCUGAAUACAAACCAUCGGAUCGUGGUGUGCAGCGAGAAUAGCUUGCAG
UUCUUCAUGCCCGUGUUGGGUGCCCUGUUCAUCGGUGUGGCUGUGGCCCCAGC
UAACGACAUCUACAACGAGCGCGAGCUGCUGAACAGCAUGGGCAUCAGCCAGC
CCACCGUCGUAUUCGUGAGCAAGAAAGGGCUGCAAAAGAUCCUCAACGUGCAA
AAGAAGCUACCGAUCAUACAAAAGAUCAUCAUCAUGGAUAGCAAGACCGACU
ACCAGGGCUUCCAAAGCAUGUACACCUUCGUGACUUCCCAUUUGCCACCCGGC
UUCAACGAGUACGACUUCGUGCCCGAGAGCUUCGACCGGGACAAAACCAUCGC
CCUGAUCAUGAACAGUAGUGGCAGUACCGGAUUGCCCAAGGGCGUAGCCCUAC
CGCACCGCACCGCUUGUGUCCGAUUCAGUCAUGCCCGCGACCCCAUCUUCGGC
AACCAGAUCAUCCCCGACACCGCUAUCCUCAGCGUGGUGCCAUUUCACCACGG
CUUCGGCAUGUUCACCACGCUGGGCUACUUGAUCUGCGGCUUUCGGGUCGUGC
UCAUGUACCGCUUCGAGGAGGAGCUAUUCUUGCGCAGCUUGCAAGACUAUAA
GAUUCAAUCUGCCCUGCUGGUGCCCACACUAUUUAGCUUCUUCGCUAAGAGCA
CUCUCAUCGACAAGUACGACCUAAGCAACUUGCACGAGAUCGCCAGCGGCGGG
GCGCCGCUCAGCAAGGAGGUAGGUGAGGCCGUGGCCAAACGCUUCCACCUACC
AGGCAUCCGACAGGGCUACGGCCUGACAGAAACAACCAGCGCCAUUCUGAUCA
CCCCCGAAGGGGACGACAAGCCUGGCGCAGUAGGCAAGGUGGUGCCCUUCUUC
GAGGCUAAGGUGGUGGACUUGGACACCGGUAAGACACUGGGUGUGAACCAGC
GCGGCGAGCUGUGCGUCCGUGGCCCCAUGAUCAUGAGCGGCUACGUUAACAAC
CCCGAGGCUACAAACGCUCUCAUCGACAAGGACGGCUGGCUGCACAGCGGCGA
CAUCGCCUACUGGGACGAGGACGAGCACUUCUUCAUCGUGGACCGGCUGAAGU
CCCUGAUCAAAUACAAGGGCUACCAGGUAGCCCCAGCCGAACUGGAGAGCAUC
CUGCUGCAACACCCCAACAUCUUCGACGCCGGGGUCGCCGGCCUGCCCGACGA
CGAUGCCGGCGAGCUGCCCGCCGCAGUCGUCGUGCUGGAACACGGUAAAACCA
UGACCGAGAAGGAGAUCGUGGACUAUGUGGCCAGCCAGGUUACAACCGCCAAG
AAGCUGCGCGGUGGUGUUGUGUUCGUGGACGAGGUGCCUAAAGGACUGACCG
GCAAGUUGGACGCCCGCAAGAUCCGCGAGAUUCUCAUUAAGGCCAAGAAGGGC
GGCAAGAUCGCCGUGUAACUCGAGCCGGAAACGCAAUAGCCGAAAAACAAAAA
ACAAAAAAAACAAAAAAAAAACCAAAAAAACAAAACACAUUAAAACAGCCUG
UGGGUUGAUCCCACCCACAGGCCCAUUGGGCGCUAGCACUCUGGUAUCACGGU
ACCUUUGUGCGCCUGUUUUAUACCCCCUCCCCCAACUGUAACUUAGAAGUAAC
ACACACCGAUCAACAGUCAGCGUGGCACACCAGCCACGUUUUGAUCAAGCACU
UCUGUUACCCCGGACUGAGUAUCAAUAGACUGCUCACGCGGUUGAAGGAGAA
AGCGUUCGUUAUCCGGCCAACUACUUCGAAAAACCUAGUAACACCGUGGAAGU
UGCAGAGUGUUUCGCUCAGCACUACCCCAGUGUAGAUCAGGUCGAUGAGUCAC
CGCAUUCCCCACGGGCGACCGUGGCGGUGGCUGCGUUGGCGGCCUGCCCAUGG
GGAAACCCAUGGGACGCUCUAAUACAGACAUGGUGCGAAGAGUCUAUUGAGC
UAGUUGGUAGUCCUCCGGCCCCUGAAUGCGGCUAAUCCUAACUGCGGAGCACA
CACCCUCAAGCCAGAGGGCAGUGUGUCGUAACGGGCAACUCUGCAGCGGAACC
GACUACUUUGGGUGUCCGUGUUUCAUUUUAUUCCUAUACUGGCUGCUUAUGG
UGACAAUUGAGAGAUCGUUACCAUAUAGCUAUUGGAUUGGCCAUCCGGUGAC
UAAUAGAGCUAUUAUAUAUCCCUUUGUUGGGUUUAUACCACUUAGCUUGAAA
GAGGUUAAAACAUUACAAUUCAUUGUUAAGUUGAAUACAGCAAAAUGAGCAA
GAUCUACAUCGACGAGCGGAGCAACGCCGAGAUCGUGUGCGAGGCCAUCAAGA
CCAUCGGCAUCGAGGGCGCCACCGCCGCCCAGCUGACCAGGCAGCUGAACAUG
GAGAAGCGGGAGGUGAACAAGGCCCUGUACGACCUGCAGAGGAGCGCUAUGG
UGUACUCCAGCGACGACAUCCCUCCCCGGUGGUUCAUGACCACCGAGGCCGAC
AAGCCCGACGCCGACGCUAUGGCCGACGUGAUCAUCGACGACGUGAGCAGGGA
GAAGUCCAUGAGGGAGGACCACAAGAGCUUCGACGACGUGAUCCCCGCCAAGA
AGAUCAUCGACUGGAAGGGCGCCAACCCCGUGACCGUGAUCAACGAGUACUGC
CAGAUCACCAGGAGGGACUGGAGCUUCCGGAUCGAGAGCGUGGGCCCCAGCAA
CAGCCCCACCUUCUACGCCUGCGUGGACAUCGACGGCAGGGUGUUCGACAAGG
CCGACGGCAAGAGCAAGCGGGACGCCAAGAACAACGCCGCCAAGCUGGCCGUG
GACAAGCUGCUGGGCUACGUGAUCAUCCGGUUCUAAACGUAUGUUACGUGCA
AAGGUGAUUGUCACCCCCCGAAAGACCAUAUUGUGACACACCCUCAGUAUCAC
GCCCAAACAUUUACAGCCGCGGUGUCAAAAACCGCGUGGACGUGGUUAACAUC
CCUGCUGGGAGGAUCAGCCGUAAUUAUUAUAAUUGGCUUGGUGCUGGCUACU
AUUGUGGCCAUGUACGUGCUGACCAACCAGAAACAUAAUUGAAUACAGCAGC
AAUUGGCAAGCUGCUUACAUAGAACUCGCGGCGAUUGGCAUGCCGCCUUAAAA
UUUUUAUUUUAUUUUUUCUUUUCUUUUCCGAAUCGGAUUUUGUUUUUAAUAU
UUCAAAAAAAAAAAAAAAAAAAAAAAAAUCUAGAAAAAAAAAAAAAAAAAAA
AAAAAAAAAAAAAAAAAAAAAAAAAAAAAAAAAAAAAAAAAAAAAAAAAAAA
AAAAAAAAAAAAAAAAAAAAAAAAAAAAA
(RNA sequence for STARR Fluc IRES-E3L (short 3′ UTR))
SEQ ID NO: 119
AUGGGCGGCGCAUGAGAGAAGCCCAGACCAAUUACCUACCCAAAAUGGAGAAA
GUUCACGUUGACAUCGAGGAAGACAGCCCAUUCCUCAGAGCUUUGCAGCGGAG
CUUCCCGCAGUUUGAGGUAGAAGCCAAGCAGGUCACUGAUAAUGACCAUGCUA
AUGCCAGAGCGUUUUCGCAUCUGGCUUCAAAACUGAUCGAAACGGAGGUGGA
CCCAUCCGACACGAUCCUUGACAUUGGAAGUGCGCCCGCCCGCAGAAUGUAUU
CUAAGCACAAGUAUCAUUGUAUCUGUCCGAUGAGAUGUGCGGAAGAUCCGGA
CAGAUUGUAUAAGUAUGCAACUAAGCUGAAGAAAAACUGUAAGGAAAUAACU
GAUAAGGAAUUGGACAAGAAAAUGAAGGAGCUGGCCGCCGUCAUGAGCGACC
CUGACCUGGAAACUGAGACUAUGUGCCUCCACGACGACGAGUCGUGUCGCUAC
GAAGGGCAAGUCGCUGUUUACCAGGAUGUAUACGCCGUCGACGGCCCCACCAG
CCUGUACCACCAGGCCAACAAGGGCGUGAGGGUGGCCUACUGGAUCGGCUUCG
ACACCACACCCUUCAUGUUCAAGAACCUGGCCGGCGCCUACCCCAGCUACAGC
ACCAACUGGGCCGACGAGACCGUGCUGACCGCCAGGAACAUCGGCCUGUGCAG
CAGCGACGUGAUGGAGAGGAGCCGGAGAGGCAUGAGCAUCCUGAGGAAGAAA
UACCUGAAGCCCAGCAACAACGUGCUGUUCAGCGUGGGCAGCACCAUCUACCA
CGAGAAGAGGGACCUGCUCAGGAGCUGGCACCUGCCCAGCGUGUUCCACCUGA
GGGGCAAGCAGAACUACACCUGCAGGUGCGAGACCAUCGUGAGCUGCGACGGC
UACGUGGUGAAGAGGAUCGCCAUCAGCCCCGGCCUGUACGGCAAGCCCAGCGG
CUACGCCGCUACAAUGCACAGGGAGGGCUUCCUGUGCUGCAAGGUGACCGACA
CCCUGAACGGCGAGAGGGUGAGCUUCCCCGUGUGCACCUACGUGCCCGCCACC
CUGUGCGACCAGAUGACCGGCAUCCUGGCCACCGACGUGAGCGCCGACGACGC
CCAGAAGCUGCUCGUGGGCCUGAACCAGAGGAUCGUGGUCAACGGCAGGACCC
AGAGGAACACCAACACAAUGAAGAACUACCUGCUGCCCGUGGUGGCCCAGGCU
UUCGCCAGGUGGGCCAAGGAGUACAAGGAGGACCAGGAAGACGAGAGGCCCCU
GGGCCUGAGGGACAGGCAGCUGGUGAUGGGCUGCUGCUGGGCCUUCAGGCGGC
ACAAGAUCACCAGCAUCUACAAGAGGCCCGACACCCAGACCAUCAUCAAGGUG
AACAGCGACUUCCACAGCUUCGUGCUGCCCAGGAUCGGCAGCAACACCCUGGA
GAUCGGCCUGAGGACCCGGAUCAGGAAGAUGCUGGAGGAACACAAGGAGCCCA
GCCCACUGAUCACCGCCGAGGACGUGCAGGAGGCCAAGUGCGCUGCCGACGAG
GCCAAGGAGGUGAGGGAGGCCGAGGAACUGAGGGCCGCCCUGCCACCCCUGGC
UGCCGACGUGGAGGAACCCACCCUGGAAGCCGACGUGGACCUGAUGCUGCAGG
AGGCCGGCGCCGGAAGCGUGGAGACACCCAGGGGCCUGAUCAAGGUGACCAGC
UACGACGGCGAGGACAAGAUCGGCAGCUACGCCGUGCUGAGCCCACAGGCCGU
GCUGAAGUCCGAGAAGCUGAGCUGCAUCCACCCACUGGCCGAGCAGGUGAUCG
UGAUCACCCACAGCGGCAGGAAGGGCAGGUACGCCGUGGAGCCCUACCACGGC
AAGGUGGUCGUGCCCGAGGGCCACGCCAUCCCCGUGCAGGACUUCCAGGCCCU
GAGCGAGAGCGCCACCAUCGUGUACAACGAGAGGGAGUUCGUGAACAGGUACC
UGCACCAUAUCGCCACCCACGGCGGAGCCCUGAACACCGACGAGGAAUACUAC
AAGACCGUGAAGCCCAGCGAGCACGACGGCGAGUACCUGUACGACAUCGACAG
GAAGCAGUGCGUGAAGAAAGAGCUGGUGACCGGCCUGGGACUGACCGGCGAG
CUGGUGGACCCACCCUUCCACGAGUUCGCCUACGAGAGCCUGAGGACCAGACC
CGCCGCUCCCUACCAGGUGCCCACCAUCGGCGUGUACGGCGUGCCCGGCAGCG
GAAAGAGCGGCAUCAUCAAGAGCGCCGUGACCAAGAAAGACCUGGUGGUCAGC
GCCAAGAAAGAGAACUGCGCCGAGAUCAUCAGGGACGUGAAGAAGAUGAAAG
GCCUGGACGUGAACGCGCGCACCGUGGACAGCGUGCUGCUGAACGGCUGCAAG
CACCCCGUGGAGACCCUGUACAUCGACGAGGCCUUCGCUUGCCACGCCGGCAC
CCUGAGGGCCCUGAUCGCCAUCAUCAGGCCCAAGAAAGCCGUGCUGUGCGGCG
ACCCCAAGCAGUGCGGCUUCUUCAACAUGAUGUGCCUGAAGGUGCACUUCAAC
CACGAGAUCUGCACCCAGGUGUUCCACAAGAGCAUCAGCAGGCGGUGCACCAA
GAGCGUGACCAGCGUCGUGAGCACCCUGUUCUACGACAAGAAAAUGAGGACCA
CCAACCCCAAGGAGACCAAAAUCGUGAUCGACACCACAGGCAGCACCAAGCCC
AAGCAGGACGACCUGAUCCUGACCUGCUUCAGGGGCUGGGUGAAGCAGCUGCA
GAUCGACUACAAGGGCAACGAGAUCAUGACCGCCGCUGCCAGCCAGGGCCUGA
CCAGGAAGGGCGUGUACGCCGUGAGGUACAAGGUGAACGAGAACCCACUGUAC
GCUCCCACCAGCGAGCACGUGAACGUGCUGCUGACCAGGACCGAGGACAGGAU
CGUGUGGAAGACCCUGGCCGGCGACCCCUGGAUCAAGACCCUGACCGCCAAGU
ACCCCGGCAACUUCACCGCCACCAUCGAAGAGUGGCAGGCCGAGCACGACGCC
AUCAUGAGGCACAUCCUGGAGAGGCCCGACCCCACCGACGUGUUCCAGAACAA
GGCCAACGUGUGCUGGGCCAAGGCCCUGGUGCCCGUGCUGAAGACCGCCGGCA
UCGACAUGACCACAGAGCAGUGGAACACCGUGGACUACUUCGAGACCGACAAG
GCCCACAGCGCCGAGAUCGUGCUGAACCAGCUGUGCGUGAGGUUCUUCGGCCU
GGACCUGGACAGCGGCCUGUUCAGCGCCCCCACCGUGCCACUGAGCAUCAGGA
ACAACCACUGGGACAACAGCCCCAGCCCAAACAUGUACGGCCUGAACAAGGAG
GUGGUCAGGCAGCUGAGCAGGCGGUACCCACAGCUGCCCAGGGCCGUGGCCAC
CGGCAGGGUGUACGACAUGAACACCGGCACCCUGAGGAACUACGACCCCAGGA
UCAACCUGGUGCCCGUGAACAGGCGGCUGCCCCACGCCCUGGUGCUGCACCAC
AACGAGCACCCACAGAGCGACUUCAGCUCCUUCGUGAGCAAGCUGAAAGGCAG
GACCGUGCUGGUCGUGGGCGAGAAGCUGAGCGUGCCCGGCAAGAUGGUGGAC
UGGCUGAGCGACAGGCCCGAGGCCACCUUCCGGGCCAGGCUGGACCUCGGCAU
CCCCGGCGACGUGCCCAAGUACGACAUCAUCUUCGUGAACGUCAGGACCCCAU
ACAAGUACCACCAUUACCAGCAGUGCGAGGACCACGCCAUCAAGCUGAGCAUG
CUGACCAAGAAGGCCUGCCUGCACCUGAACCCCGGAGGCACCUGCGUGAGCAU
CGGCUACGGCUACGCCGACAGGGCCAGCGAGAGCAUCAUUGGCGCCAUCGCCA
GGCUGUUCAAGUUCAGCAGGGUGUGCAAACCCAAGAGCAGCCUGGAGGAAACC
GAGGUGCUGUUCGUGUUCAUCGGCUACGACCGGAAGGCCAGGACCCACAACCC
CUACAAGCUGAGCAGCACCCUGACAAACAUCUACACCGGCAGCAGGCUGCACG
AGGCCGGCUGCGCCCCCAGCUACCACGUGGUCAGGGGCGAUAUCGCCACCGCC
ACCGAGGGCGUGAUCAUCAACGCUGCCAACAGCAAGGGCCAGCCCGGAGGCGG
AGUGUGCGGCGCCCUGUACAAGAAGUUCCCCGAGAGCUUCGACCUGCAGCCCA
UCGAGGUGGGCAAGGCCAGGCUGGUGAAGGGCGCCGCUAAGCACAUCAUCCAC
GCCGUGGGCCCCAACUUCAACAAGGUGAGCGAGGUGGAAGGCGACAAGCAGCU
GGCCGAAGCCUACGAGAGCAUCGCCAAGAUCGUGAACGACAAUAACUACAAGA
GCGUGGCCAUCCCACUGCUCAGCACCGGCAUCUUCAGCGGCAACAAGGACAGG
CUGACCCAGAGCCUGAACCACCUGCUCACCGCCCUGGACACCACCGAUGCCGA
CGUGGCCAUCUACUGCAGGGACAAGAAGUGGGAGAUGACCCUGAAGGAGGCC
GUGGCCAGGCGGGAGGCCGUGGAAGAGAUCUGCAUCAGCGACGACUCCAGCGU
GACCGAGCCCGACGCCGAGCUGGUGAGGGUGCACCCCAAGAGCUCCCUGGCCG
GCAGGAAGGGCUACAGCACCAGCGACGGCAAGACCUUCAGCUACCUGGAGGGC
ACCAAGUUCCACCAGGCCGCUAAGGACAUCGCCGAGAUCAACGCUAUGUGGCC
CGUGGCCACCGAGGCCAACGAGCAGGUGUGCAUGUACAUCCUGGGCGAGAGCA
UGUCCAGCAUCAGGAGCAAGUGCCCCGUGGAGGAAAGCGAGGCCAGCACACCA
CCCAGCACCCUGCCCUGCCUGUGCAUCCACGCUAUGACACCCGAGAGGGUGCA
GCGGCUGAAGGCCAGCAGGCCCGAGCAGAUCACCGUGUGCAGCUCCUUCCCAC
UGCCCAAGUACAGGAUCACCGGCGUGCAGAAGAUCCAGUGCAGCCAGCCCAUC
CUGUUCAGCCCAAAGGUGCCCGCCUACAUCCACCCCAGGAAGUACCUGGUGGA
GACCCCACCCGUGGACGAGACACCCGAGCCAAGCGCCGAGAACCAGAGCACCG
AGGGCACACCCGAGCAGCCACCCCUGAUCACCGAGGACGAGACAAGGACCCGG
ACCCCAGAGCCCAUCAUUAUCGAGGAAGAGGAAGAGGACAGCAUCAGCCUGCU
GAGCGACGGCCCCACCCACCAGGUGCUGCAGGUGGAGGCCGACAUCCACGGCC
CACCCAGCGUGUCCAGCUCCAGCUGGAGCAUCCCACACGCCAGCGACUUCGAC
GUGGACAGCCUGAGCAUCCUGGACACCCUGGAGGGCGCCAGCGUGACCUCCGG
CGCCACCAGCGCCGAGACCAACAGCUACUUCGCCAAGAGCAUGGAGUUCCUGG
CCAGGCCCGUGCCAGCUCCCAGGACCGUGUUCAGGAACCCACCCCACCCAGCUC
CCAGGACCAGGACCCCAAGCCUGGCUCCCAGCAGGGCCUGCAGCAGGACCAGC
CUGGUGAGCACCCCACCCGGCGUGAACAGGGUGAUCACCAGGGAGGAACUGGA
GGCCCUGACACCCAGCAGGACCCCCAGCAGGUCCGUGAGCAGGACUAGUCUGG
UGUCCAACCCACCCGGCGUGAACAGGGUGAUCACCAGGGAGGAAUUCGAGGCC
UUCGUGGCCCAGCAACAGAGACGGUUCGACGCCGGCGCCUACAUCUUCAGCAG
CGACACCGGCCAGGGACACCUGCAGCAAAAGAGCGUGAGGCAGACCGUGCUGA
GCGAGGUGGUGCUGGAGAGGACCGAGCUGGAAAUCAGCUACGCCCCCAGGCUG
GACCAGGAGAAGGAGGAACUGCUCAGGAAGAAACUGCAGCUGAACCCCACCCC
AGCCAACAGGAGCAGGUACCAGAGCAGGAAGGUGGAGAACAUGAAGGCCAUC
ACCGCCAGGCGGAUCCUGCAGGGCCUGGGACACUACCUGAAGGCCGAGGGCAA
GGUGGAGUGCUACAGGACCCUGCACCCCGUGCCACUGUACAGCUCCAGCGUGA
ACAGGGCCUUCUCCAGCCCCAAGGUGGCCGUGGAGGCCUGCAACGCUAUGCUG
AAGGAGAACUUCCCCACCGUGGCCAGCUACUGCAUCAUCCCCGAGUACGACGC
CUACCUGGACAUGGUGGACGGCGCCAGCUGCUGCCUGGACACCGCCAGCUUCU
GCCCCGCCAAGCUGAGGAGCUUCCCCAAGAAACACAGCUACCUGGAGCCCACC
AUCAGGAGCGCCGUGCCCAGCGCCAUCCAGAACACCCUGCAGAACGUGCUGGC
CGCUGCCACCAAGAGGAACUGCAACGUGACCCAGAUGAGGGAGCUGCCCGUGC
UGGACAGCGCUGCCUUCAACGUGGAGUGCUUCAAGAAAUACGCCUGCAACAAC
GAGUACUGGGAGACCUUCAAGGAGAACCCCAUCAGGCUGACCGAAGAGAACGU
GGUGAACUACAUCACCAAGCUGAAGGGCCCCAAGGCCGCUGCCCUGUUCGCUA
AGACCCACAACCUGAACAUGCUGCAGGACAUCCCAAUGGACAGGUUCGUGAUG
GACCUGAAGAGGGACGUGAAGGUGACACCCGGCACCAAGCACACCGAGGAGAG
GCCCAAGGUGCAGGUGAUCCAGGCCGCUGACCCACUGGCCACCGCCUACCUGU
GCGGCAUCCACAGGGAGCUGGUGAGGCGGCUGAACGCCGUGCUGCUGCCCAAC
AUCCACACCCUGUUCGACAUGAGCGCCGAGGACUUCGACGCCAUCAUCGCCGA
GCACUUCCAGCCCGGCGACUGCGUGCUGGAGACCGACAUCGCCAGCUUCGACA
AGAGCGAGGAUGACGCUAUGGCCCUGACCGCUCUGAUGAUCCUGGAGGACCUG
GGCGUGGACGCCGAGCUGCUCACCCUGAUCGAGGCUGCCUUCGGCGAGAUCAG
CUCCAUCCACCUGCCCACCAAGACCAAGUUCAAGUUCGGCGCUAUGAUGAAAA
GCGGAAUGUUCCUGACCCUGUUCGUGAACACCGUGAUCAACAUUGUGAUCGCC
AGCAGGGUGCUGCGGGAGAGGCUGACCGGCAGCCCCUGCGCUGCCUUCAUCGG
CGACGACAACAUCGUGAAGGGCGUGAAAAGCGACAAGCUGAUGGCCGACAGG
UGCGCCACCUGGCUGAACAUGGAGGUGAAGAUCAUCGACGCCGUGGUGGGCGA
GAAGGCCCCCUACUUCUGCGGCGGAUUCAUCCUGUGCGACAGCGUGACCGGCA
CCGCCUGCAGGGUGGCCGACCCCCUGAAGAGGCUGUUCAAGCUGGGCAAGCCA
CUGGCCGCUGACGAUGAGCACGACGAUGACAGGCGGAGGGCCCUGCACGAGGA
AAGCACCAGGUGGAACAGGGUGGGCAUCCUGAGCGAGCUGUGCAAGGCCGUG
GAGAGCAGGUACGAGACCGUGGGCACCAGCAUCAUCGUGAUGGCUAUGACCAC
ACUGGCCAGCUCCGUCAAGAGCUUCUCCUACCUGAGGGGGGCCCCUAUAACUC
UCUACGGCUAACCUGAAUGGACUACGACAUAGUCUAGUCCGCCAAGGCCGCCA
CCAUGGAAGAUGCCAAAAACAUUAAGAAGGGCCCAGCGCCAUUCUACCCACUC
GAAGACGGGACCGCCGGCGAGCAGCUGCACAAAGCCAUGAAGCGCUACGCCCU
GGUGCCCGGCACCAUCGCCUUUACCGACGCACAUAUCGAGGUGGACAUUACCU
ACGCCGAGUACUUCGAGAUGAGCGUUCGGCUGGCAGAAGCUAUGAAGCGCUA
UGGGCUGAAUACAAACCAUCGGAUCGUGGUGUGCAGCGAGAAUAGCUUGCAG
UUCUUCAUGCCCGUGUUGGGUGCCCUGUUCAUCGGUGUGGCUGUGGCCCCAGC
UAACGACAUCUACAACGAGCGCGAGCUGCUGAACAGCAUGGGCAUCAGCCAGC
CCACCGUCGUAUUCGUGAGCAAGAAAGGGCUGCAAAAGAUCCUCAACGUGCAA
AAGAAGCUACCGAUCAUACAAAAGAUCAUCAUCAUGGAUAGCAAGACCGACU
ACCAGGGCUUCCAAAGCAUGUACACCUUCGUGACUUCCCAUUUGCCACCCGGC
UUCAACGAGUACGACUUCGUGCCCGAGAGCUUCGACCGGGACAAAACCAUCGC
CCUGAUCAUGAACAGUAGUGGCAGUACCGGAUUGCCCAAGGGCGUAGCCCUAC
CGCACCGCACCGCUUGUGUCCGAUUCAGUCAUGCCCGCGACCCCAUCUUCGGC
AACCAGAUCAUCCCCGACACCGCUAUCCUCAGCGUGGUGCCAUUUCACCACGG
CUUCGGCAUGUUCACCACGCUGGGCUACUUGAUCUGCGGCUUUCGGGUCGUGC
UCAUGUACCGCUUCGAGGAGGAGCUAUUCUUGCGCAGCUUGCAAGACUAUAA
GAUUCAAUCUGCCCUGCUGGUGCCCACACUAUUUAGCUUCUUCGCUAAGAGCA
CUCUCAUCGACAAGUACGACCUAAGCAACUUGCACGAGAUCGCCAGCGGCGGG
GCGCCGCUCAGCAAGGAGGUAGGUGAGGCCGUGGCCAAACGCUUCCACCUACC
AGGCAUCCGACAGGGCUACGGCCUGACAGAAACAACCAGCGCCAUUCUGAUCA
CCCCCGAAGGGGACGACAAGCCUGGCGCAGUAGGCAAGGUGGUGCCCUUCUUC
GAGGCUAAGGUGGUGGACUUGGACACCGGUAAGACACUGGGUGUGAACCAGC
GCGGCGAGCUGUGCGUCCGUGGCCCCAUGAUCAUGAGCGGCUACGUUAACAAC
CCCGAGGCUACAAACGCUCUCAUCGACAAGGACGGCUGGCUGCACAGCGGCGA
CAUCGCCUACUGGGACGAGGACGAGCACUUCUUCAUCGUGGACCGGCUGAAGU
CCCUGAUCAAAUACAAGGGCUACCAGGUAGCCCCAGCCGAACUGGAGAGCAUC
CUGCUGCAACACCCCAACAUCUUCGACGCCGGGGUCGCCGGCCUGCCCGACGA
CGAUGCCGGCGAGCUGCCCGCCGCAGUCGUCGUGCUGGAACACGGUAAAACCA
UGACCGAGAAGGAGAUCGUGGACUAUGUGGCCAGCCAGGUUACAACCGCCAAG
AAGCUGCGCGGUGGUGUUGUGUUCGUGGACGAGGUGCCUAAAGGACUGACCG
GCAAGUUGGACGCCCGCAAGAUCCGCGAGAUUCUCAUUAAGGCCAAGAAGGGC
GGCAAGAUCGCCGUGUAACUCGAGCCGGAAACGCAAUAGCCGAAAAACAAAAA
ACAAAAAAAACAAAAAAAAAACCAAAAAAACAAAACACAUUAAAACAGCCUG
UGGGUUGAUCCCACCCACAGGCCCAUUGGGCGCUAGCACUCUGGUAUCACGGU
ACCUUUGUGCGCCUGUUUUAUACCCCCUCCCCCAACUGUAACUUAGAAGUAAC
ACACACCGAUCAACAGUCAGCGUGGCACACCAGCCACGUUUUGAUCAAGCACU
UCUGUUACCCCGGACUGAGUAUCAAUAGACUGCUCACGCGGUUGAAGGAGAA
AGCGUUCGUUAUCCGGCCAACUACUUCGAAAAACCUAGUAACACCGUGGAAGU
UGCAGAGUGUUUCGCUCAGCACUACCCCAGUGUAGAUCAGGUCGAUGAGUCAC
CGCAUUCCCCACGGGCGACCGUGGCGGUGGCUGCGUUGGCGGCCUGCCCAUGG
GGAAACCCAUGGGACGCUCUAAUACAGACAUGGUGCGAAGAGUCUAUUGAGC
UAGUUGGUAGUCCUCCGGCCCCUGAAUGCGGCUAAUCCUAACUGCGGAGCACA
CACCCUCAAGCCAGAGGGCAGUGUGUCGUAACGGGCAACUCUGCAGCGGAACC
GACUACUUUGGGUGUCCGUGUUUCAUUUUAUUCCUAUACUGGCUGCUUAUGG
UGACAAUUGAGAGAUCGUUACCAUAUAGCUAUUGGAUUGGCCAUCCGGUGAC
UAAUAGAGCUAUUAUAUAUCCCUUUGUUGGGUUUAUACCACUUAGCUUGAAA
GAGGUUAAAACAUUACAAUUCAUUGUUAAGUUGAAUACAGCAAAAUGAGCAA
GAUCUACAUCGACGAGCGGAGCAACGCCGAGAUCGUGUGCGAGGCCAUCAAGA
CCAUCGGCAUCGAGGGCGCCACCGCCGCCCAGCUGACCAGGCAGCUGAACAUG
GAGAAGCGGGAGGUGAACAAGGCCCUGUACGACCUGCAGAGGAGCGCUAUGG
UGUACUCCAGCGACGACAUCCCUCCCCGGUGGUUCAUGACCACCGAGGCCGAC
AAGCCCGACGCCGACGCUAUGGCCGACGUGAUCAUCGACGACGUGAGCAGGGA
GAAGUCCAUGAGGGAGGACCACAAGAGCUUCGACGACGUGAUCCCCGCCAAGA
AGAUCAUCGACUGGAAGGGCGCCAACCCCGUGACCGUGAUCAACGAGUACUGC
CAGAUCACCAGGAGGGACUGGAGCUUCCGGAUCGAGAGCGUGGGCCCCAGCAA
CAGCCCCACCUUCUACGCCUGCGUGGACAUCGACGGCAGGGUGUUCGACAAGG
CCGACGGCAAGAGCAAGCGGGACGCCAAGAACAACGCCGCCAAGCUGGCCGUG
GACAAGCUGCUGGGCUACGUGAUCAUCCGGUUCUAAACAAUUGGCAAGCUGCU
UACAUAGAACUCGCGGCGAUUGGCAUGCCGCCUUAAAAUUUUUAUUUUAUUU
UUUCUUUUCUUUUCCGAAUCGGAUUUUGUUUUUAAUAUUUCAAAAAAAAAAA
AAAAAAAAAAAAAAUCUAGAAAAAAAAAAAAAAAAAAAAAAAAAAAAAAAAA
AAAAAAAAAAAAAAAAAAAAAAAAAAAAAAAAAAAAAAAAAAAAAAAAAAAA
AAAAAAAAAAAAAAA

1. A nucleic acid molecule comprising:
(i) a first polynucleotide encoding one or more viral replication proteins, wherein the first polynucleotide is codon-optimized as compared to a wild-type polynucleotide encoding the one or more viral replication proteins; and
(ii) a second polynucleotide comprising a first transgene encoding a first antigenic protein or a fragment thereof; and
(iii) a 5′ UTR comprising a sequence of SEQ ID NO:73 or SEQ ID NO:74.

2. The nucleic acid molecule of claim 1, wherein the one or more viral replication proteins are alphavirus proteins or rubivirus proteins.

3. The nucleic acid molecule of claim 2, wherein the alphavirus proteins are from Venezuelan Equine Encephalitis Virus (VEEV), Eastern Equine Encephalitis Virus (EEEV), Everglades Virus (EVEV), Mucambo Virus (MUCV), Semliki Forest Virus (SFV), Pixuna Virus (PIXV), Middleburg Virus (MIDV), Chikungunya Virus (CHIKV), O'Nyong-Nyong Virus (ONNV), Ross River Virus (RRV), Barmah Forest Virus (BFV), Getah Virus (GETV), Sagiyama Virus (SAGV), Bebaru Virus (BEBV), Mayaro Virus (MAYV), Una Virus (UNAV), Sindbis Virus (SINV), Aura Virus (AURAV), Whataroa Virus (WHAV), Babanki Virus (BABV), Kyzylagach Virus (KYZV), Western Equine Encephalitis Virus (WEEV), Highland J Virus (HJV), Fort Morgan Virus (FMV), Ndumu Virus (NDUV), Salmonid Alphavirus (SAV), Buggy Creek Virus (BCRV), or any combination thereof.

4. The nucleic acid molecule of claim 1, wherein the first polynucleotide encodes a polyprotein comprising an alphavirus nsP1 protein, an alphavirus nsP2 protein, an alphavirus nsP3 protein, an alphavirus nsP4 protein, or any combination thereof.

5. (canceled)

6. (canceled)

7. (canceled)

8. (canceled)

9. (canceled)

10. (canceled)

11. (canceled)

12. (canceled)

13. (canceled)

14. The nucleic acid molecule of claim 1, further comprising a 3′ untranslated region (UTR).

15. (canceled)

16. (canceled)

17. (canceled)

18. (canceled)

19. (canceled)

20. The nucleic acid molecule of claim 1, wherein the first antigenic protein is a viral protein, a bacterial protein, a fungal protein, a protozoan protein, a parasite protein, or a tumor protein.

21. The nucleic acid molecule of claim 20, wherein the viral protein is an orthomyxovirus protein, a paramyxovirus protein, a picornavirus protein, a flavivirus protein, a filovirus protein, a rhabdovirus protein, a togavirus protein, an arterivirus protein, a bunyavirus protein, an arenavirus protein, a reovirus protein, a bornavirus protein, a retrovirus protein, an adenovirus protein, a herpesvirus protein, a polyomavirus protein, a papillomavirus protein, a poxvirus protein, or a hepadnavirus protein.

22. The nucleic acid molecule of claim 20, wherein the first antigenic protein is an influenza virus protein, a respiratory syncytial virus (RSV) protein, a human immunodeficiency virus (HIV) protein, a hepatitis C virus (HCV) protein, a cytomegalovirus (CMV) protein, a Lassa Fever Virus (LFV) protein, an Ebola Virus (EBOV) protein, a Mycobacterium protein, a Bacillus protein, a Yersinia protein, a Streptococcus protein, a Pseudomonas protein, a Shigella protein, a Campylobacter protein, a Salmonella protein, a Plasmodium protein, or a Toxoplasma protein.

23. The nucleic acid molecule of claim 20, wherein the tumor protein is a kidney cancer, renal cancer, urinary bladder cancer, prostate cancer, uterine cancer, breast cancer, cervical cancer, ovarian cancer, lung cancer, liver cancer, stomach cancer, colon cancer, rectal cancer, oral cavity cancer, pharynx cancer, pancreatic cancer, thyroid cancer, melanoma, skin cancer, head and neck cancer, brain cancer, hematopoietic cancer, leukemia, lymphoma, bone cancer, or sarcoma protein.

24. The nucleic acid molecule of claim 1, wherein the second polynucleotide comprises at least two transgenes.

25. The nucleic acid molecule of claim 24, wherein a second transgene encodes a second antigenic protein or a fragment thereof, in an immunomodulatory protein, or a reporter protein.

26. The nucleic acid molecule of claim 24, wherein the second polynucleotide further comprises a sequence encoding a 2A peptide, an internal ribosomal entry site (IRES), a subgenomic promoter, or a combination thereof, located between transgenes.

27. (canceled)

28. The nucleic acid molecule of claim 24, wherein the first and second transgenes encode viral proteins, bacterial proteins, fungal proteins, protozoan proteins, parasite proteins, tumor proteins, immunomodulatory proteins, reporter proteins, or any combination thereof.

29. The nucleic acid molecule of claim 1, wherein the first polynucleotide is located 5′ of the second polynucleotide.

30. The nucleic acid molecule of claim 29, further comprising an intergenic region located between the first polynucleotide and the second polynucleotide.

31. The nucleic acid molecule of claim 30, wherein the intergenic region comprises a sequence having at least 85% identity to a sequence of SEQ ID NO:77.

32. The nucleic acid molecule of claim 1, wherein the nucleic acid molecule is
(a) a DNA molecule; or
(b) an RNA molecule, wherein T is substituted with U.

33. The nucleic acid molecule of claim 32, wherein the DNA molecule further comprises a promoter located 5′ of the 5′ UTR, wherein the promoter is a T7 promoter, a T3 promoter, or an SP6 promoter.

34. (canceled)

35. (canceled)

36. The nucleic acid molecule of claim 32, wherein the RNA molecule is a self-replicating RNA molecule.

37. The nucleic acid molecule of claim 32, wherein the RNA molecule further comprises a 5′ cap having a Cap 1 structure, a Cap 1 (^{m6}A) structure, a Cap 2 structure, or a Cap 0 structure.

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. A pharmaceutical composition comprising the nucleic acid molecule of claim 1 and a lipid formulation.

77. The pharmaceutical composition of claim 76, wherein the lipid formulation comprises an ionizable cationic lipid.

78. The pharmaceutical composition of claim 77, wherein
(a) the ionizable cationic lipid has a structure of Formula I: 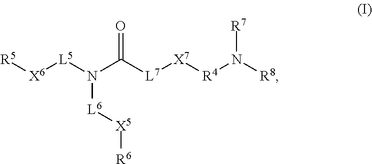
or a pharmaceutically acceptable salt thereof,
wherein R⁵ and R⁶ are each independently selected from the group consisting of a linear or branched C₁-C₃₁ alkyl, C₂-C₃₁ alkenyl or C₂-C₃₁ alkynyl and cholesteryl; L⁵ and L⁶ are each independently selected from the group consisting of a linear C₁-C₂₀ alkyl and C₂-C₂₀ alkenyl; X⁵ is —C(O)O—, whereby —C(O)O—R⁶ is formed or —OC(O)— whereby —OC(O)—R ⁶ is formed; X⁶ is —C(O)O— whereby —C(O)O—R⁵ is formed or —OC(O)— whereby —OC(O)—R ⁵ is formed; X⁷ is S or O; L⁷ is absent or lower alkyl; R⁴ is a linear or branched C₁-C₆ alkyl; and R⁷ and R⁸ are each independently selected from the group consisting of a hydrogen and a linear or branched C₁-C₆ alkyl; or
(b) the ionizable cationic lipid has a structure of 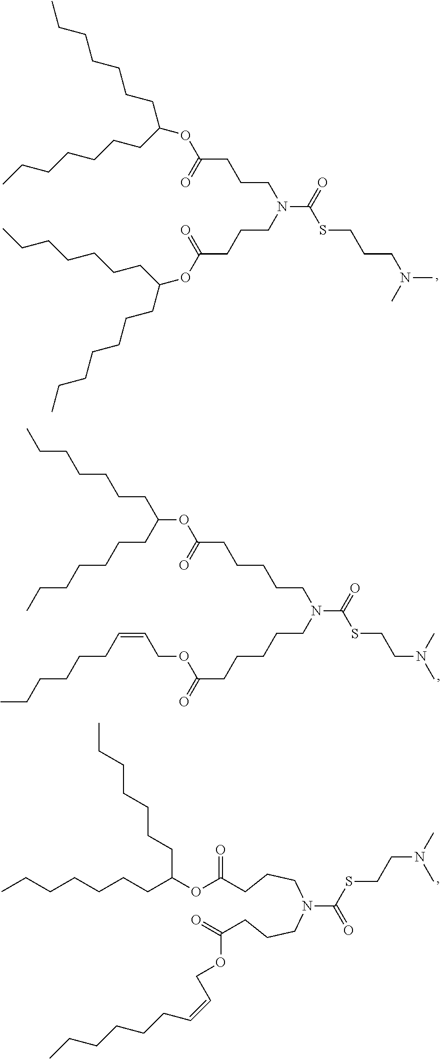
or a pharmaceutically acceptable salt thereof.

79. A method of inducing an immune response in a subject comprising:
administering to the subject an effective amount of a nucleic acid molecule of of claim 1, thereby inducing an immune response to the first antigenic protein.

80. (canceled)

81. (canceled)

82. (canceled)

83. A method of inducing an immune response in a subject comprising:
administering to the subject an effective amount of a pharmaceutical composition of claim 76, thereby inducing an immune response to the first antigenic protein.

84. (canceled)

85. (canceled)

86. (canceled)


SEQUENCE LISTING (119 sequences)

SEQ ID NO: 1 - DNA, 18 bp, synthetic construct
  misc_feature 1..18: note = Synthetic polynucleotide
  source 1..18: mol_type = other DNA
  modified_base 15: mod_base = OTHER; note = uracil
gaggaaacttaagatggg

SEQ ID NO: 2-4 - skipped (3 entries)

SEQ ID NO: 5 - RNA, 129 nt, synthetic construct
  misc_feature 1..129: note = Synthetic polynucleotide
  source 1..129: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatag

SEQ ID NO: 6 - RNA, 28 nt, synthetic construct
  misc_feature 1..28: note = Synthetic polynucleotide
  source 1..28: mol_type = other RNA
attattacatcaaaacaaaaagccgcca

SEQ ID NO: 7 - RNA, 245 nt, synthetic construct
  misc_feature 1..245: note = Synthetic polynucleotide
  source 1..245: mol_type = other RNA
cttaagggggcgctgcctacggaggtggcagccatctccttctcggcatcaagcttaccatggtgccccaggccctgctcttggtcccgctgctggtgttccccctctgcttcggcaagttccccatctacaccatccccgacaagctggggccgtggagccccatcgacatccaccacctgtcctgccccaacaacctcgtggtcgaggacgagggctgcaccaacctgagcgggttctcctac

SEQ ID NO: 8 - RNA, 177 nt, synthetic construct
  misc_feature 1..177: note = Synthetic polynucleotide
  source 1..177: mol_type = other RNA
tgagtgtcgtacagcctccaggcccccccctcccgggagagccatagtggtctgcggaaccggtgagtacaccggaattgccgggaagactgggtcctttcttggataaacccactctatgcccggccatttgggcgtgcccccgcaagactgctagccgagtagtgttgggttgcg

SEQ ID NO: 9 - RNA, 89 nt, synthetic construct
  misc_feature 1..89: note = Synthetic polynucleotide
  source 1..89: mol_type = other RNA
aattattggttaaagaagtatattagtgctaatttccctccgtttgtcctagcttttctcttctgtcaaccccacacgcctttggcaca

SEQ ID NO: 10 - RNA, 569 nt, synthetic construct
  misc_feature 1..569: note = Synthetic polynucleotide
  source 1..569: mol_type = other RNA
ctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacgtgtgtttagtcgaggttaaaaaacgtctaggccccccgaaccacggggacgtggttttcctttgaaaaacacgatgataat

SEQ ID NO: 11 - RNA, 44 nt, synthetic construct
  misc_feature 1..44: note = Synthetic polynucleotide
  source 1..44: mol_type = other RNA
gtcagctttcaaactctttgtttcttgtttgttgattgagaata

SEQ ID NO: 12 - RNA, 54 nt, synthetic construct
  misc_feature 1..54: note = Synthetic polynucleotide
  source 1..54: mol_type = other RNA
ctctcgcctgagaaaaaaaatccacgaaccaatttctcagcaaccagcagcacg

SEQ ID NO: 13 - RNA, 52 nt, synthetic construct
  misc_feature 1..52: note = Synthetic polynucleotide
  source 1..52: mol_type = other RNA
acctgtgagggttcgaaggaagtagcagtgttttttgttcctagaggaagag

SEQ ID NO: 14 - RNA, 71 nt, synthetic construct
  misc_feature 1..71: note = Synthetic polynucleotide
  source 1..71: mol_type = other RNA
acacagaaacattcgcaaaaacaaaatcccagtatcaaaattcttctctttttttcatatttcgcaaagac

SEQ ID NO: 15 - RNA, 52 nt, synthetic construct
  misc_feature 1..52: note = Synthetic polynucleotide
  source 1..52: mol_type = other RNA
cagaaaaatttgctacattgtttcacaaacttcaaatattattcatttattt

SEQ ID NO: 16 - RNA, 158 nt, synthetic construct
  misc_feature 1..158: note = Synthetic polynucleotide
  source 1..158: mol_type = other RNA
ctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacat

SEQ ID NO: 17 - RNA, 166 nt, synthetic construct
  misc_feature 1..166: note = Synthetic polynucleotide
  source 1..166: mol_type = other RNA
tgcaaggctggccggaagcccttgcctgaaagcaagatttcagcctggaagagggcaaagtggacgggagtggacaggagtggatgcgataagatgtggtttgaagctgatgggtgccagccctgcattgctgagtcaatcaataaagagctttcttttgacccat

SEQ ID NO: 18 - RNA, 143 nt, synthetic construct
  misc_feature 1..143: note = Synthetic polynucleotide
  source 1..143: mol_type = other RNA
acgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgca

SEQ ID NO: 19 - RNA, 220 nt, synthetic construct
  misc_feature 1..220: note = Synthetic polynucleotide
  source 1..220: mol_type = other RNA
tagagcggcaaaccctagctacactccatagctagtttcttttttttttgtttttttttttttttttttttttttttttttttttttttcctttcttttccttctttttttcctcttttcttggtggctccatcttagccctagtcacggctagctgtgaaaggtccgtgagccgcatgactgcagagagtgccgtaactggtctctctgcagatcatgt

SEQ ID NO: 20 - RNA, 170 nt, synthetic construct
  misc_feature 1..170: note = Synthetic polynucleotide
  source 1..170: mol_type = other RNA
acacatcacaaccacaaccttctcaggctaccctgagaaaaaaagacatgaagactcaggactcatcttttctgttggtgtaaaatcaacaccctaaggaacacaaatttctttaaacatttgacttcttgtctctgtgctgcaattaataaaaaatggaaagaatctac

SEQ ID NO: 21 - RNA, 110 nt, synthetic construct
  misc_feature 1..110: note = Synthetic polynucleotide
  source 1..110: mol_type = other RNA
gctggagcctcggtagccgttcctcctgcccgctgggcctcccaacgggccctcctcccctccttgcaccggcccttcctggtctttgaataaagtctgagtgggcagca

SEQ ID NO: 22 - RNA, 123 nt, synthetic construct
  misc_feature 1..123: note = Synthetic polynucleotide
  source 1..123: mol_type = other RNA
tagtgcagtcactggcacaacgcgttgcccggtaagccaatcgggtatacacggtcgtcatactgcagacagggttcttctactttgcaagatagtctagagtagtaaaataaatagtataag

SEQ ID NO: 23-24 - skipped (2 entries)

SEQ ID NO: 25 - RNA, 21 nt, synthetic construct
  misc_feature 1..21: note = Synthetic polynucleotide
  source 1..21: mol_type = other RNA
cacaaagagtaaagaagaaca

SEQ ID NO: 26 - RNA, 21 nt, synthetic construct
  misc_feature 1..21: note = Synthetic polynucleotide
  source 1..21: mol_type = other RNA
aacactaaaagtagaagaaaa

SEQ ID NO: 27 - RNA, 21 nt, synthetic construct
  misc_feature 1..21: note = Synthetic polynucleotide
  source 1..21: mol_type = other RNA
ctcagaaagataagatcagcc

SEQ ID NO: 28 - RNA, 21 nt, synthetic construct
  misc_feature 1..21: note = Synthetic polynucleotide
  source 1..21: mol_type = other RNA
aaccaatcgaaagaaaccaaa

SEQ ID NO: 29 - RNA, 21 nt, synthetic construct
  misc_feature 1..21: note = Synthetic polynucleotide
  source 1..21: mol_type = other RNA
ctctaatcaccaggagtaaaa

SEQ ID NO: 30 - RNA, 21 nt, synthetic construct
  misc_feature 1..21: note = Synthetic polynucleotide
  source 1..21: mol_type = other RNA
gagagagatcttaacaaaaaa

SEQ ID NO: 31 - RNA, 21 nt, synthetic construct
  misc_feature 1..21: note = Synthetic polynucleotide
  source 1..21: mol_type = other RNA
tgtgtaacaacaacaacaaca

SEQ ID NO: 32 - RNA, 21 nt, synthetic construct
  misc_feature 1..21: note = Synthetic polynucleotide
  source 1..21: mol_type = other RNA
ccgcagtaggaagagaaagcc

SEQ ID NO: 33 - RNA, 21 nt, synthetic construct
  misc_feature 1..21: note = Synthetic polynucleotide
  source 1..21: mol_type = other RNA
aaaaaaaaaagaaatcataaa

SEQ ID NO: 34 - RNA, 21 nt, synthetic construct
  misc_feature 1..21: note = Synthetic polynucleotide
  source 1..21: mol_type = other RNA
gagagaagaaagaagaagacg

SEQ ID NO: 35 - RNA, 21 nt, synthetic construct
  misc_feature 1..21: note = Synthetic polynucleotide
  source 1..21: mol_type = other RNA
caattaaaaatacttaccaaa

SEQ ID NO: 36 - RNA, 21 nt, synthetic construct
  misc_feature 1..21: note = Synthetic polynucleotide
  source 1..21: mol_type = other RNA
gcaaacagagtaagcgaaacg

SEQ ID NO: 37 - RNA, 21 nt, synthetic construct
  misc_feature 1..21: note = Synthetic polynucleotide
  source 1..21: mol_type = other RNA
gcgaagaagacgaacgcaaag

SEQ ID NO: 38 - RNA, 21 nt, synthetic construct
  misc_feature 1..21: note = Synthetic polynucleotide
  source 1..21: mol_type = other RNA
ttaggactgtattgactggcc

SEQ ID NO: 39 - RNA, 21 nt, synthetic construct
  misc_feature 1..21: note = Synthetic polynucleotide
  source 1..21: mol_type = other RNA
atcatcggaattcggaaaaag

SEQ ID NO: 40 - RNA, 21 nt, synthetic construct
  misc_feature 1..21: note = Synthetic polynucleotide
  source 1..21: mol_type = other RNA
aaaacaaaagttaaagcagac

SEQ ID NO: 41 - RNA, 21 nt, synthetic construct
  misc_feature 1..21: note = Synthetic polynucleotide
  source 1..21: mol_type = other RNA
tttatctcaaataagaaggca

SEQ ID NO: 42 - RNA, 21 nt, synthetic construct
  misc_feature 1..21: note = Synthetic polynucleotide
  source 1..21: mol_type = other RNA
ggtggggaggtgagatttctt

SEQ ID NO: 43 - RNA, 21 nt, synthetic construct
  misc_feature 1..21: note = Synthetic polynucleotide
  source 1..21: mol_type = other RNA
tgattaggaaactacaaagcc

SEQ ID NO: 44 - RNA, 21 nt, synthetic construct
  misc_feature 1..21: note = Synthetic polynucleotide
  source 1..21: mol_type = other RNA
catttttcaatttcataaaac

SEQ ID NO: 45 - RNA, 21 nt, synthetic construct
  misc_feature 1..21: note = Synthetic polynucleotide
  source 1..21: mol_type = other RNA
ttacttttaagcccaacaaaa

SEQ ID NO: 46 - RNA, 21 nt, synthetic construct
  misc_feature 1..21: note = Synthetic polynucleotide
  source 1..21: mol_type = other RNA
ggcgtgtgtgtgtgttgttga

SEQ ID NO: 47 - RNA, 21 nt, synthetic construct
  misc_feature 1..21: note = Synthetic polynucleotide
  source 1..21: mol_type = other RNA
gtggtgaaggggaaggtttag

SEQ ID NO: 48 - RNA, 21 nt, synthetic construct
  misc_feature 1..21: note = Synthetic polynucleotide
  source 1..21: mol_type = other RNA
ttgtttttttttggtttggtt

SEQ ID NO: 49 - DNA, 44 bp, synthetic construct
  misc_feature 1..44: note = Synthetic polynucleotide
  source 1..44: mol_type = other DNA
atgggcggcgcatgagagaagcccagaccaattacctacccaaa

SEQ ID NO: 50 - DNA, 7482 bp, synthetic construct
  misc_feature 1..7482: note = Synthetic polynucleotide
  source 1..7482: mol_type = other DNA
atggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgccgtcgacggccccaccagcctgtaccaccaggccaacaagggcgtgagggtggcctactggatcggcttcgacaccacacccttcatgttcaagaacctggccggcgcctaccccagctacagcaccaactgggccgacgagaccgtgctgaccgccaggaacatcggcctgtgcagcagcgacgtgatggagaggagccggagaggcatgagcatcctgaggaagaaatacctgaagcccagcaacaacgtgctgttcagcgtgggcagcaccatctaccacgagaagagggacctgctcaggagctggcacctgcccagcgtgttccacctgaggggcaagcagaactacacctgcaggtgcgagaccatcgtgagctgcgacggctacgtggtgaagaggatcgccatcagccccggcctgtacggcaagcccagcggctacgccgctacaatgcacagggagggcttcctgtgctgcaaggtgaccgacaccctgaacggcgagagggtgagcttccccgtgtgcacctacgtgcccgccaccctgtgcgaccagatgaccggcatcctggccaccgacgtgagcgccgacgacgcccagaagctgctcgtgggcctgaaccagaggatcgtggtcaacggcaggacccagaggaacaccaacacaatgaagaactacctgctgcccgtggtggcccaggctttcgccaggtgggccaaggagtacaaggaggaccaggaagacgagaggcccctgggcctgagggacaggcagctggtgatgggctgctgctgggccttcaggcggcacaagatcaccagcatctacaagaggcccgacacccagaccatcatcaaggtgaacagcgacttccacagcttcgtgctgcccaggatcggcagcaacaccctggagatcggcctgaggacccggatcaggaagatgctggaggaacacaaggagcccagcccactgatcaccgccgaggacgtgcaggaggccaagtgcgctgccgacgaggccaaggaggtgagggaggccgaggaactgagggccgccctgccacccctggctgccgacgtggaggaacccaccctggaagccgacgtggacctgatgctgcaggaggccggcgccggaagcgtggagacacccaggggcctgatcaaggtgaccagctacgacggcgaggacaagatcggcagctacgccgtgctgagcccacaggccgtgctgaagtccgagaagctgagctgcatccacccactggccgagcaggtgatcgtgatcacccacagcggcaggaagggcaggtacgccgtggagccctaccacggcaaggtggtcgtgcccgagggccacgccatccccgtgcaggacttccaggccctgagcgagagcgccaccatcgtgtacaacgagagggagttcgtgaacaggtacctgcaccatatcgccacccacggcggagccctgaacaccgacgaggaatactacaagaccgtgaagcccagcgagcacgacggcgagtacctgtacgacatcgacaggaagcagtgcgtgaagaaagagctggtgaccggcctgggactgaccggcgagctggtggacccacccttccacgagttcgcctacgagagcctgaggaccagacccgccgctccctaccaggtgcccaccatcggcgtgtacggcgtgcccggcagcggaaagagcggcatcatcaagagcgccgtgaccaagaaagacctggtggtcagcgccaagaaagagaactgcgccgagatcatcagggacgtgaagaagatgaaaggcctggacgtgaacgcgcgcaccgtggacagcgtgctgctgaacggctgcaagcaccccgtggagaccctgtacatcgacgaggccttcgcttgccacgccggcaccctgagggccctgatcgccatcatcaggcccaagaaagccgtgctgtgcggcgaccccaagcagtgcggcttcttcaacatgatgtgcctgaaggtgcacttcaaccacgagatctgcacccaggtgttccacaagagcatcagcaggcggtgcaccaagagcgtgaccagcgtcgtgagcaccctgttctacgacaagaaaatgaggaccaccaaccccaaggagaccaaaatcgtgatcgacaccacaggcagcaccaagcccaagcaggacgacctgatcctgacctgcttcaggggctgggtgaagcagctgcagatcgactacaagggcaacgagatcatgaccgccgctgccagccagggcctgaccaggaagggcgtgtacgccgtgaggtacaaggtgaacgagaacccactgtacgctcccaccagcgagcacgtgaacgtgctgctgaccaggaccgaggacaggatcgtgtggaagaccctggccggcgacccctggatcaagaccctgaccgccaagtaccccggcaacttcaccgccaccatcgaagagtggcaggccgagcacgacgccatcatgaggcacatcctggagaggcccgaccccaccgacgtgttccagaacaaggccaacgtgtgctgggccaaggccctggtgcccgtgctgaagaccgccggcatcgacatgaccacagagcagtggaacaccgtggactacttcgagaccgacaaggcccacagcgccgagatcgtgctgaaccagctgtgcgtgaggttcttcggcctggacctggacagcggcctgttcagcgcccccaccgtgccactgagcatcaggaacaaccactgggacaacagccccagcccaaacatgtacggcctgaacaaggaggtggtcaggcagctgagcaggcggtacccacagctgcccagggccgtggccaccggcagggtgtacgacatgaacaccggcaccctgaggaactacgaccccaggatcaacctggtgcccgtgaacaggcggctgccccacgccctggtgctgcaccacaacgagcacccacagagcgacttcagctccttcgtgagcaagctgaaaggcaggaccgtgctggtcgtgggcgagaagctgagcgtgcccggcaagatggtggactggctgagcgacaggcccgaggccaccttccgggccaggctggacctcggcatccccggcgacgtgcccaagtacgacatcatcttcgtgaacgtcaggaccccatacaagtaccaccattaccagcagtgcgaggaccacgccatcaagctgagcatgctgaccaagaaggcctgcctgcacctgaaccccggaggcacctgcgtgagcatcggctacggctacgccgacagggccagcgagagcatcattggcgccatcgccaggctgttcaagttcagcagggtgtgcaaacccaagagcagcctggaggaaaccgaggtgctgttcgtgttcatcggctacgaccggaaggccaggacccacaacccctacaagctgagcagcaccctgacaaacatctacaccggcagcaggctgcacgaggccggctgcgcccccagctaccacgtggtcaggggcgatatcgccaccgccaccgagggcgtgatcatcaacgctgccaacagcaagggccagcccggaggcggagtgtgcggcgccctgtacaagaagttccccgagagcttcgacctgcagcccatcgaggtgggcaaggccaggctggtgaagggcgccgctaagcacatcatccacgccgtgggccccaacttcaacaaggtgagcgaggtggaaggcgacaagcagctggccgaagcctacgagagcatcgccaagatcgtgaacgacaataactacaagagcgtggccatcccactgctcagcaccggcatcttcagcggcaacaaggacaggctgacccagagcctgaaccacctgctcaccgccctggacaccaccgatgccgacgtggccatctactgcagggacaagaagtgggagatgaccctgaaggaggccgtggccaggcgggaggccgtggaagagatctgcatcagcgacgactccagcgtgaccgagcccgacgccgagctggtgagggtgcaccccaagagctccctggccggcaggaagggctacagcaccagcgacggcaagaccttcagctacctggagggcaccaagttccaccaggccgctaaggacatcgccgagatcaacgctatgtggcccgtggccaccgaggccaacgagcaggtgtgcatgtacatcctgggcgagagcatgtccagcatcaggagcaagtgccccgtggaggaaagcgaggccagcacaccacccagcaccctgccctgcctgtgcatccacgctatgacacccgagagggtgcagcggctgaaggccagcaggcccgagcagatcaccgtgtgcagctccttcccactgcccaagtacaggatcaccggcgtgcagaagatccagtgcagccagcccatcctgttcagcccaaaggtgcccgcctacatccaccccaggaagtacctggtggagaccccacccgtggacgagacacccgagccaagcgccgagaaccagagcaccgagggcacacccgagcagccacccctgatcaccgaggacgagacaaggacccggaccccagagcccatcattatcgaggaagaggaagaggacagcatcagcctgctgagcgacggccccacccaccaggtgctgcaggtggaggccgacatccacggcccacccagcgtgtccagctccagctggagcatcccacacgccagcgacttcgacgtggacagcctgagcatcctggacaccctggagggcgccagcgtgacctccggcgccaccagcgccgagaccaacagctacttcgccaagagcatggagttcctggccaggcccgtgccagctcccaggaccgtgttcaggaacccaccccacccagctcccaggaccaggaccccaagcctggctcccagcagggcctgcagcaggaccagcctggtgagcaccccacccggcgtgaacagggtgatcaccagggaggaactggaggccctgacacccagcaggacccccagcaggtccgtgagcaggactagtctggtgtccaacccacccggcgtgaacagggtgatcaccagggaggaattcgaggccttcgtggcccagcaacagagacggttcgacgccggcgcctacatcttcagcagcgacaccggccagggacacctgcagcaaaagagcgtgaggcagaccgtgctgagcgaggtggtgctggagaggaccgagctggaaatcagctacgcccccaggctggaccaggagaaggaggaactgctcaggaagaaactgcagctgaaccccaccccagccaacaggagcaggtaccagagcaggaaggtggagaacatgaaggccatcaccgccaggcggatcctgcagggcctgggacactacctgaaggccgagggcaaggtggagtgctacaggaccctgcaccccgtgccactgtacagctccagcgtgaacagggccttctccagccccaaggtggccgtggaggcctgcaacgctatgctgaaggagaacttccccaccgtggccagctactgcatcatccccgagtacgacgcctacctggacatggtggacggcgccagctgctgcctggacaccgccagcttctgccccgccaagctgaggagcttccccaagaaacacagctacctggagcccaccatcaggagcgccgtgcccagcgccatccagaacaccctgcagaacgtgctggccgctgccaccaagaggaactgcaacgtgacccagatgagggagctgcccgtgctggacagcgctgccttcaacgtggagtgcttcaagaaatacgcctgcaacaacgagtactgggagaccttcaaggagaaccccatcaggctgaccgaagagaacgtggtgaactacatcaccaagctgaagggccccaaggccgctgccctgttcgctaagacccacaacctgaacatgctgcaggacatcccaatggacaggttcgtgatggacctgaagagggacgtgaaggtgacacccggcaccaagcacaccgaggagaggcccaaggtgcaggtgatccaggccgctgacccactggccaccgcctacctgtgcggcatccacagggagctggtgaggcggctgaacgccgtgctgctgcccaacatccacaccctgttcgacatgagcgccgaggacttcgacgccatcatcgccgagcacttccagcccggcgactgcgtgctggagaccgacatcgccagcttcgacaagagcgaggatgacgctatggccctgaccgctctgatgatcctggaggacctgggcgtggacgccgagctgctcaccctgatcgaggctgccttcggcgagatcagctccatccacctgcccaccaagaccaagttcaagttcggcgctatgatgaaaagcggaatgttcctgaccctgttcgtgaacaccgtgatcaacattgtgatcgccagcagggtgctgcgggagaggctgaccggcagcccctgcgctgccttcatcggcgacgacaacatcgtgaagggcgtgaaaagcgacaagctgatggccgacaggtgcgccacctggctgaacatggaggtgaagatcatcgacgccgtggtgggcgagaaggccccctacttctgcggcggattcatcctgtgcgacagcgtgaccggcaccgcctgcagggtggccgaccccctgaagaggctgttcaagctgggcaagccactggccgctgacgatgagcacgacgatgacaggcggagggccctgcacgaggaaagcaccaggtggaacagggtgggcatcctgagcgagctgtgcaaggccgtggagagcaggtacgagaccgtgggcaccagcatcatcgtgatggctatgaccacactggccagctccgtcaagagcttctcctacctgaggggggcccctataactctctacggctaa

SEQ ID NO: 51 - protein, 2493 aa, synthetic construct
  REGION 1..2493: note = Synthetic polypeptide
MEKVHVDIEEDSPFLRALQRSFPQFEVEAKQVTDNDHANARAFSHLASKLIETEVDPSDTILDIGSAPARRMYSKHKYHCICPMRCAEDPDRLYKYATKLKKNCKEITDKELDKKMKELAAVMSDPDLETETMCLHDDESCRYEGQVAVYQDVYAVDGPTSLYHQANKGVRVAYWIGFDTTPFMFKNLAGAYPSYSTNWADETVLTARNIGLCSSDVMERSRRGMSILRKKYLKPSNNVLFSVGSTIYHEKRDLLRSWHLPSVFHLRGKQNYTCRCETIVSCDGYVVKRIAISPGLYGKPSGYAATMHREGFLCCKVTDTLNGERVSFPVCTYVPATLCDQMTGILATDVSADDAQKLLVGLNQRIVVNGRTQRNTNTMKNYLLPVVAQAFARWAKEYKEDQEDERPLGLRDRQLVMGCCWAFRRHKITSIYKRPDTQTIIKVNSDFHSFVLPRIGSNTLEIGLRTRIRKMLEEHKEPSPLITAEDVQEAKCAADEAKEVREAEELRAALPPLAADVEEPTLEADVDLMLQEAGAGSVETPRGLIKVTSYDGEDKIGSYAVLSPQAVLKSEKLSCIHPLAEQVIVITHSGRKGRYAVEPYHGKVVVPEGHAIPVQDFQALSESATIVYNEREFVNRYLHHIATHGGALNTDEEYYKTVKPSEHDGEYLYDIDRKQCVKKELVTGLGLTGELVDPPFHEFAYESLRTRPAAPYQVPTIGVYGVPGSGKSGIIKSAVTKKDLVVSAKKENCAEIIRDVKKMKGLDVNARTVDSVLLNGCKHPVETLYIDEAFACHAGTLRALIAIIRPKKAVLCGDPKQCGFFNMMCLKVHFNHEICTQVFHKSISRRCTKSVTSVVSTLFYDKKMRTTNPKETKIVIDTTGSTKPKQDDLILTCFRGWVKQLQIDYKGNEIMTAAASQGLTRKGVYAVRYKVNENPLYAPTSEHVNVLLTRTEDRIVWKTLAGDPWIKTLTAKYPGNFTATIEEWQAEHDAIMRHILERPDPTDVFQNKANVCWAKALVPVLKTAGIDMTTEQWNTVDYFETDKAHSAEIVLNQLCVRFFGLDLDSGLFSAPTVPLSIRNNHWDNSPSPNMYGLNKEVVRQLSRRYPQLPRAVATGRVYDMNTGTLRNYDPRINLVPVNRRLPHALVLHHNEHPQSDFSSFVSKLKGRTVLVVGEKLSVPGKMVDWLSDRPEATFRARLDLGIPGDVPKYDIIFVNVRTPYKYHHYQQCEDHAIKLSMLTKKACLHLNPGGTCVSIGYGYADRASESIIGAIARLFKFSRVCKPKSSLEETEVLFVFIGYDRKARTHNPYKLSSTLTNIYTGSRLHEAGCAPSYHVVRGDIATATEGVIINAANSKGQPGGGVCGALYKKFPESFDLQPIEVGKARLVKGAAKHIIHAVGPNFNKVSEVEGDKQLAEAYESIAKIVNDNNYKSVAIPLLSTGIFSGNKDRLTQSLNHLLTALDTTDADVAIYCRDKKWEMTLKEAVARREAVEEICISDDSSVTEPDAELVRVHPKSSLAGRKGYSTSDGKTFSYLEGTKFHQAAKDIAEINAMWPVATEANEQVCMYILGESMSSIRSKCPVEESEASTPPSTLPCLCIHAMTPERVQRLKASRPEQITVCSSFPLPKYRITGVQKIQCSQPILFSPKVPAYIHPRKYLVETPPVDETPEPSAENQSTEGTPEQPPLITEDETRTRTPEPIIIEEEEEDSISLLSDGPTHQVLQVEADIHGPPSVSSSSWSIPHASDFDVDSLSILDTLEGASVTSGATSAETNSYFAKSMEFLARPVPAPRTVFRNPPHPAPRTRTPSLAPSRACSRTSLVSTPPGVNRVITREELEALTPSRTPSRSVSRTSLVSNPPGVNRVITREEFEAFVAQQQRRFDAGAYIFSSDTGQGHLQQKSVRQTVLSEVVLERTELEISYAPRLDQEKEELLRKKLQLNPTPANRSRYQSRKVENMKAITARRILQGLGHYLKAEGKVECYRTLHPVPLYSSSVNRAFSSPKVAVEACNAMLKENFPTVASYCIIPEYDAYLDMVDGASCCLDTASFCPAKLRSFPKKHSYLEPTIRSAVPSAIQNTLQNVLAAATKRNCNVTQMRELPVLDSAAFNVECFKKYACNNEYWETFKENPIRLTEENVVNYITKLKGPKAAALFAKTHNLNMLQDIPMDRFVMDLKRDVKVTPGTKHTEERPKVQVIQAADPLATAYLCGIHRELVRRLNAVLLPNIHTLFDMSAEDFDAIIAEHFQPGDCVLETDIASFDKSEDDAMALTALMILEDLGVDAELLTLIEAAFGEISSIHLPTKTKFKFGAMMKSGMFLTLFVNTVINIVIASRVLRERLTGSPCAAFIGDDNIVKGVKSDKLMADRCATWLNMEVKIIDAVVGEKAPYFCGGFILCDSVTGTACRVADPLKRLFKLGKPLAADDEHDDDRRRALHEESTRWNRVGILSELCKAVESRYETVGTSIIVMAMTTLASSVKSFSYLRGAPITLYG

SEQ ID NO: 52 - DNA, 44 bp, synthetic construct
  misc_feature 1..44: note = Synthetic polynucleotide
  source 1..44: mol_type = other DNA
cctgaatggactacgacatagtctagtccgccaaggccgccacc

SEQ ID NO: 53 - DNA, 468 bp, synthetic construct
  misc_feature 1..468: note = Synthetic polynucleotide
  source 1..468: mol_type = other DNA
actcgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaatctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 54 - DNA, 7485 bp, synthetic construct
  misc_feature 1..7485: note = Synthetic polynucleotide
  source 1..7485: mol_type = other DNA
atgcccgagaaggtgcacgtggacatcgaggaggacagccccttcctgagggccctgcagaggagcttcccacagttcgaagtggaggccaagcaggtgaccgacaacgaccacgccaacgccagggccttcagccacctggccagcaagctgatcgagaccgaggtggaccccagcgacaccatcctggacatcggcagcgccccagccaggagaatgtacagcaagcacaagtaccactgcatctgccccatgaggtgcgccgaggaccccgacaggctgtacaagtacgccaccaaactgaagaagaactgcaaggagatcaccgacaaggagctggacaagaaaatgaaggagctggccgccgtgatgagcgaccccgacctggagaccgagacaatgtgcctgcacgacgacgagagctgcaggtacgagggccaggtggccgtctaccaggacgtgtacgccgtcgacggccccaccagcctgtaccaccaggccaacaagggcgtgagggtggcctactggatcggcttcgacaccacacccttcatgttcaagaacctggccggcgcctaccccagctacagcaccaactgggccgacgagaccgtgctgaccgccaggaacatcggcctgtgcagcagcgacgtgatggagaggagccggagaggcatgagcatcctgaggaagaaatacctgaagcccagcaacaacgtgctgttcagcgtgggcagcaccatctaccacgagaagagggacctgctcaggagctggcacctgcccagcgtgttccacctgaggggcaagcagaactacacctgcaggtgcgagaccatcgtgagctgcgacggctacgtggtgaagaggatcgccatcagccccggcctgtacggcaagcccagcggctacgccgctacaatgcacagggagggcttcctgtgctgcaaggtgaccgacaccctgaacggcgagagggtgagcttccccgtgtgcacctacgtgcccgccaccctgtgcgaccagatgaccggcatcctggccaccgacgtgagcgccgacgacgcccagaagctgctcgtgggcctgaaccagaggatcgtggtcaacggcaggacccagaggaacaccaacacaatgaagaactacctgctgcccgtggtggcccaggctttcgccaggtgggccaaggagtacaaggaggaccaggaagacgagaggcccctgggcctgagggacaggcagctggtgatgggctgctgctgggccttcaggcggcacaagatcaccagcatctacaagaggcccgacacccagaccatcatcaaggtgaacagcgacttccacagcttcgtgctgcccaggatcggcagcaacaccctggagatcggcctgaggacccggatcaggaagatgctggaggaacacaaggagcccagcccactgatcaccgccgaggacgtgcaggaggccaagtgcgctgccgacgaggccaaggaggtgagggaggccgaggaactgagggccgccctgccacccctggctgccgacgtggaggaacccaccctggaagccgacgtggacctgatgctgcaggaggccggcgccggaagcgtggagacacccaggggcctgatcaaggtgaccagctacgacggcgaggacaagatcggcagctacgccgtgctgagcccacaggccgtgctgaagtccgagaagctgagctgcatccacccactggccgagcaggtgatcgtgatcacccacagcggcaggaagggcaggtacgccgtggagccctaccacggcaaggtggtcgtgcccgagggccacgccatccccgtgcaggacttccaggccctgagcgagagcgccaccatcgtgtacaacgagagggagttcgtgaacaggtacctgcaccatatcgccacccacggcggagccctgaacaccgacgaggaatactacaagaccgtgaagcccagcgagcacgacggcgagtacctgtacgacatcgacaggaagcagtgcgtgaagaaagagctggtgaccggcctgggactgaccggcgagctggtggacccacccttccacgagttcgcctacgagagcctgaggaccagacccgccgctccctaccaggtgcccaccatcggcgtgtacggcgtgcccggcagcggaaagagcggcatcatcaagagcgccgtgaccaagaaagacctggtggtcagcgccaagaaagagaactgcgccgagatcatcagggacgtgaagaagatgaaaggcctggacgtgaacgcgcgcaccgtggacagcgtgctgctgaacggctgcaagcaccccgtggagaccctgtacatcgacgaggccttcgcttgccacgccggcaccctgagggccctgatcgccatcatcaggcccaagaaagccgtgctgtgcggcgaccccaagcagtgcggcttcttcaacatgatgtgcctgaaggtgcacttcaaccacgagatctgcacccaggtgttccacaagagcatcagcaggcggtgcaccaagagcgtgaccagcgtcgtgagcaccctgttctacgacaagaaaatgaggaccaccaaccccaaggagaccaaaatcgtgatcgacaccacaggcagcaccaagcccaagcaggacgacctgatcctgacctgcttcaggggctgggtgaagcagctgcagatcgactacaagggcaacgagatcatgaccgccgctgccagccagggcctgaccaggaagggcgtgtacgccgtgaggtacaaggtgaacgagaacccactgtacgctcccaccagcgagcacgtgaacgtgctgctgaccaggaccgaggacaggatcgtgtggaagaccctggccggcgacccctggatcaagaccctgaccgccaagtaccccggcaacttcaccgccaccatcgaagagtggcaggccgagcacgacgccatcatgaggcacatcctggagaggcccgaccccaccgacgtgttccagaacaaggccaacgtgtgctgggccaaggccctggtgcccgtgctgaagaccgccggcatcgacatgaccacagagcagtggaacaccgtggactacttcgagaccgacaaggcccacagcgccgagatcgtgctgaaccagctgtgcgtgaggttcttcggcctggacctggacagcggcctgttcagcgcccccaccgtgccactgagcatcaggaacaaccactgggacaacagccccagcccaaacatgtacggcctgaacaaggaggtggtcaggcagctgagcaggcggtacccacagctgcccagggccgtggccaccggcagggtgtacgacatgaacaccggcaccctgaggaactacgaccccaggatcaacctggtgcccgtgaacaggcggctgccccacgccctggtgctgcaccacaacgagcacccacagagcgacttcagctccttcgtgagcaagctgaaaggcaggaccgtgctggtcgtgggcgagaagctgagcgtgcccggcaagatggtggactggctgagcgacaggcccgaggccaccttccgggccaggctggacctcggcatccccggcgacgtgcccaagtacgacatcatcttcgtgaacgtcaggaccccatacaagtaccaccattaccagcagtgcgaggaccacgccatcaagctgagcatgctgaccaagaaggcctgcctgcacctgaaccccggaggcacctgcgtgagcatcggctacggctacgccgacagggccagcgagagcatcattggcgccatcgccaggctgttcaagttcagcagggtgtgcaaacccaagagcagcctggaggaaaccgaggtgctgttcgtgttcatcggctacgaccggaaggccaggacccacaacccctacaagctgagcagcaccctgacaaacatctacaccggcagcaggctgcacgaggccggctgcgcccccagctaccacgtggtcaggggcgatatcgccaccgccaccgagggcgtgatcatcaacgctgccaacagcaagggccagcccggaggcggagtgtgcggcgccctgtacaagaagttccccgagagcttcgacctgcagcccatcgaggtgggcaaggccaggctggtgaagggcgccgctaagcacatcatccacgccgtgggccccaacttcaacaaggtgagcgaggtggaaggcgacaagcagctggccgaagcctacgagagcatcgccaagatcgtgaacgacaataactacaagagcgtggccatcccactgctcagcaccggcatcttcagcggcaacaaggacaggctgacccagagcctgaaccacctgctcaccgccctggacaccaccgatgccgacgtggccatctactgcagggacaagaagtgggagatgaccctgaaggaggccgtggccaggcgggaggccgtggaagagatctgcatcagcgacgactccagcgtgaccgagcccgacgccgagctggtgagggtgcaccccaagagctccctggccggcaggaagggctacagcaccagcgacggcaagaccttcagctacctggagggcaccaagttccaccaggccgctaaggacatcgccgagatcaacgctatgtggcccgtggccaccgaggccaacgagcaggtgtgcatgtacatcctgggcgagagcatgtccagcatcaggagcaagtgccccgtggaggaaagcgaggccagcacaccacccagcaccctgccctgcctgtgcatccacgctatgacacccgagagggtgcagcggctgaaggccagcaggcccgagcagatcaccgtgtgcagctccttcccactgcccaagtacaggatcaccggcgtgcagaagatccagtgcagccagcccatcctgttcagcccaaaggtgcccgcctacatccaccccaggaagtacctggtggagaccccacccgtggacgagacacccgagccaagcgccgagaaccagagcaccgagggcacacccgagcagccacccctgatcaccgaggacgagacaaggacccggaccccagagcccatcattatcgaggaagaggaagaggacagcatcagcctgctgagcgacggccccacccaccaggtgctgcaggtggaggccgacatccacggcccacccagcgtgtccagctccagctggagcatcccacacgccagcgacttcgacgtggacagcctgagcatcctggacaccctggagggcgccagcgtgacctccggcgccaccagcgccgagaccaacagctacttcgccaagagcatggagttcctggccaggcccgtgccagctcccaggaccgtgttcaggaacccaccccacccagctcccaggaccaggaccccaagcctggctcccagcagggcctgcagcaggaccagcctggtgagcaccccacccggcgtgaacagggtgatcaccagggaggaactggaggccctgacacccagcaggacccccagcaggtccgtgagcaggactagtctggtgtccaacccacccggcgtgaacagggtgatcaccagggaggaattcgaggccttcgtggcccagcaacagagacggttcgacgccggcgcctacatcttcagcagcgacaccggccagggacacctgcagcaaaagagcgtgaggcagaccgtgctgagcgaggtggtgctggagaggaccgagctggaaatcagctacgcccccaggctggaccaggagaaggaggaactgctcaggaagaaactgcagctgaaccccaccccagccaacaggagcaggtaccagagcaggaaggtggagaacatgaaggccatcaccgccaggcggatcctgcagggcctgggacactacctgaaggccgagggcaaggtggagtgctacaggaccctgcaccccgtgccactgtacagctccagcgtgaacagggccttctccagccccaaggtggccgtggaggcctgcaacgctatgctgaaggagaacttccccaccgtggccagctactgcatcatccccgagtacgacgcctacctggacatggtggacggcgccagctgctgcctggacaccgccagcttctgccccgccaagctgaggagcttccccaagaaacacagctacctggagcccaccatcaggagcgccgtgcccagcgccatccagaacaccctgcagaacgtgctggccgctgccaccaagaggaactgcaacgtgacccagatgagggagctgcccgtgctggacagcgctgccttcaacgtggagtgcttcaagaaatacgcctgcaacaacgagtactgggagaccttcaaggagaaccccatcaggctgaccgaagagaacgtggtgaactacatcaccaagctgaagggccccaaggccgctgccctgttcgctaagacccacaacctgaacatgctgcaggacatcccaatggacaggttcgtgatggacctgaagagggacgtgaaggtgacacccggcaccaagcacaccgaggagaggcccaaggtgcaggtgatccaggccgctgacccactggccaccgcctacctgtgcggcatccacagggagctggtgaggcggctgaacgccgtgctgctgcccaacatccacaccctgttcgacatgagcgccgaggacttcgacgccatcatcgccgagcacttccagcccggcgactgcgtgctggagaccgacatcgccagcttcgacaagagcgaggatgacgctatggccctgaccgctctgatgatcctggaggacctgggcgtggacgccgagctgctcaccctgatcgaggctgccttcggcgagatcagctccatccacctgcccaccaagaccaagttcaagttcggcgctatgatgaaaagcggaatgttcctgaccctgttcgtgaacaccgtgatcaacattgtgatcgccagcagggtgctgcgggagaggctgaccggcagcccctgcgctgccttcatcggcgacgacaacatcgtgaagggcgtgaaaagcgacaagctgatggccgacaggtgcgccacctggctgaacatggaggtgaagatcatcgacgccgtggtgggcgagaaggccccctacttctgcggcggattcatcctgtgcgacagcgtgaccggcaccgcctgcagggtggccgaccccctgaagaggctgttcaagctgggcaagccactggccgctgacgatgagcacgacgatgacaggcggagggccctgcacgaggaaagcaccaggtggaacagggtgggcatcctgagcgagctgtgcaaggccgtggagagcaggtacgagaccgtgggcaccagcatcatcgtgatggctatgaccacactggccagctccgtcaagagcttctcctacctgaggggggcccctataactctctacggctaa

SEQ ID NO: 55 - protein, 2494 aa, synthetic construct
  REGION 1..2494: note = Synthetic polypeptide
MPEKVHVDIEEDSPFLRALQRSFPQFEVEAKQVTDNDHANARAFSHLASKLIETEVDPSDTILDIGSAPARRMYSKHKYHCICPMRCAEDPDRLYKYATKLKKNCKEITDKELDKKMKELAAVMSDPDLETETMCLHDDESCRYEGQVAVYQDVYAVDGPTSLYHQANKGVRVAYWIGFDTTPFMFKNLAGAYPSYSTNWADETVLTARNIGLCSSDVMERSRRGMSILRKKYLKPSNNVLFSVGSTIYHEKRDLLRSWHLPSVFHLRGKQNYTCRCETIVSCDGYVVKRIAISPGLYGKPSGYAATMHREGFLCCKVTDTLNGERVSFPVCTYVPATLCDQMTGILATDVSADDAQKLLVGLNQRIVVNGRTQRNTNTMKNYLLPVVAQAFARWAKEYKEDQEDERPLGLRDRQLVMGCCWAFRRHKITSIYKRPDTQTIIKVNSDFHSFVLPRIGSNTLEIGLRTRIRKMLEEHKEPSPLITAEDVQEAKCAADEAKEVREAEELRAALPPLAADVEEPTLEADVDLMLQEAGAGSVETPRGLIKVTSYDGEDKIGSYAVLSPQAVLKSEKLSCIHPLAEQVIVITHSGRKGRYAVEPYHGKVVVPEGHAIPVQDFQALSESATIVYNEREFVNRYLHHIATHGGALNTDEEYYKTVKPSEHDGEYLYDIDRKQCVKKELVTGLGLTGELVDPPFHEFAYESLRTRPAAPYQVPTIGVYGVPGSGKSGIIKSAVTKKDLVVSAKKENCAEIIRDVKKMKGLDVNARTVDSVLLNGCKHPVETLYIDEAFACHAGTLRALIAIIRPKKAVLCGDPKQCGFFNMMCLKVHFNHEICTQVFHKSISRRCTKSVTSVVSTLFYDKKMRTTNPKETKIVIDTTGSTKPKQDDLILTCFRGWVKQLQIDYKGNEIMTAAASQGLTRKGVYAVRYKVNENPLYAPTSEHVNVLLTRTEDRIVWKTLAGDPWIKTLTAKYPGNFTATIEEWQAEHDAIMRHILERPDPTDVFQNKANVCWAKALVPVLKTAGIDMTTEQWNTVDYFETDKAHSAEIVLNQLCVRFFGLDLDSGLFSAPTVPLSIRNNHWDNSPSPNMYGLNKEVVRQLSRRYPQLPRAVATGRVYDMNTGTLRNYDPRINLVPVNRRLPHALVLHHNEHPQSDFSSFVSKLKGRTVLVVGEKLSVPGKMVDWLSDRPEATFRARLDLGIPGDVPKYDIIFVNVRTPYKYHHYQQCEDHAIKLSMLTKKACLHLNPGGTCVSIGYGYADRASESIIGAIARLFKFSRVCKPKSSLEETEVLFVFIGYDRKARTHNPYKLSSTLTNIYTGSRLHEAGCAPSYHVVRGDIATATEGVIINAANSKGQPGGGVCGALYKKFPESFDLQPIEVGKARLVKGAAKHIIHAVGPNFNKVSEVEGDKQLAEAYESIAKIVNDNNYKSVAIPLLSTGIFSGNKDRLTQSLNHLLTALDTTDADVAIYCRDKKWEMTLKEAVARREAVEEICISDDSSVTEPDAELVRVHPKSSLAGRKGYSTSDGKTFSYLEGTKFHQAAKDIAEINAMWPVATEANEQVCMYILGESMSSIRSKCPVEESEASTPPSTLPCLCIHAMTPERVQRLKASRPEQITVCSSFPLPKYRITGVQKIQCSQPILFSPKVPAYIHPRKYLVETPPVDETPEPSAENQSTEGTPEQPPLITEDETRTRTPEPIIIEEEEEDSISLLSDGPTHQVLQVEADIHGPPSVSSSSWSIPHASDFDVDSLSILDTLEGASVTSGATSAETNSYFAKSMEFLARPVPAPRTVFRNPPHPAPRTRTPSLAPSRACSRTSLVSTPPGVNRVITREELEALTPSRTPSRSVSRTSLVSNPPGVNRVITREEFEAFVAQQQRRFDAGAYIFSSDTGQGHLQQKSVRQTVLSEVVLERTELEISYAPRLDQEKEELLRKKLQLNPTPANRSRYQSRKVENMKAITARRILQGLGHYLKAEGKVECYRTLHPVPLYSSSVNRAFSSPKVAVEACNAMLKENFPTVASYCIIPEYDAYLDMVDGASCCLDTASFCPAKLRSFPKKHSYLEPTIRSAVPSAIQNTLQNVLAAATKRNCNVTQMRELPVLDSAAFNVECFKKYACNNEYWETFKENPIRLTEENVVNYITKLKGPKAAALFAKTHNLNMLQDIPMDRFVMDLKRDVKVTPGTKHTEERPKVQVIQAADPLATAYLCGIHRELVRRLNAVLLPNIHTLFDMSAEDFDAIIAEHFQPGDCVLETDIASFDKSEDDAMALTALMILEDLGVDAELLTLIEAAFGEISSIHLPTKTKFKFGAMMKSGMFLTLFVNTVINIVIASRVLRERLTGSPCAAFIGDDNIVKGVKSDKLMADRCATWLNMEVKIIDAVVGEKAPYFCGGFILCDSVTGTACRVADPLKRLFKLGKPLAADDEHDDDRRRALHEESTRWNRVGILSELCKAVESRYETVGTSIIVMAMTTLASSVKSFSYLRGAPITLYG

SEQ ID NO: 56 - RNA, 9739 nt, synthetic construct
  misc_feature 1..9739: note = Synthetic polynucleotide
  source 1..9739: mol_type = other RNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgccgtcgacggccccaccagcctgtaccaccaggccaacaagggcgtgagggtggcctactggatcggcttcgacaccacacccttcatgttcaagaacctggccggcgcctaccccagctacagcaccaactgggccgacgagaccgtgctgaccgccaggaacatcggcctgtgcagcagcgacgtgatggagaggagccggagaggcatgagcatcctgaggaagaaatacctgaagcccagcaacaacgtgctgttcagcgtgggcagcaccatctaccacgagaagagggacctgctcaggagctggcacctgcccagcgtgttccacctgaggggcaagcagaactacacctgcaggtgcgagaccatcgtgagctgcgacggctacgtggtgaagaggatcgccatcagccccggcctgtacggcaagcccagcggctacgccgctacaatgcacagggagggcttcctgtgctgcaaggtgaccgacaccctgaacggcgagagggtgagcttccccgtgtgcacctacgtgcccgccaccctgtgcgaccagatgaccggcatcctggccaccgacgtgagcgccgacgacgcccagaagctgctcgtgggcctgaaccagaggatcgtggtcaacggcaggacccagaggaacaccaacacaatgaagaactacctgctgcccgtggtggcccaggctttcgccaggtgggccaaggagtacaaggaggaccaggaagacgagaggcccctgggcctgagggacaggcagctggtgatgggctgctgctgggccttcaggcggcacaagatcaccagcatctacaagaggcccgacacccagaccatcatcaaggtgaacagcgacttccacagcttcgtgctgcccaggatcggcagcaacaccctggagatcggcctgaggacccggatcaggaagatgctggaggaacacaaggagcccagcccactgatcaccgccgaggacgtgcaggaggccaagtgcgctgccgacgaggccaaggaggtgagggaggccgaggaactgagggccgccctgccacccctggctgccgacgtggaggaacccaccctggaagccgacgtggacctgatgctgcaggaggccggcgccggaagcgtggagacacccaggggcctgatcaaggtgaccagctacgacggcgaggacaagatcggcagctacgccgtgctgagcccacaggccgtgctgaagtccgagaagctgagctgcatccacccactggccgagcaggtgatcgtgatcacccacagcggcaggaagggcaggtacgccgtggagccctaccacggcaaggtggtcgtgcccgagggccacgccatccccgtgcaggacttccaggccctgagcgagagcgccaccatcgtgtacaacgagagggagttcgtgaacaggtacctgcaccatatcgccacccacggcggagccctgaacaccgacgaggaatactacaagaccgtgaagcccagcgagcacgacggcgagtacctgtacgacatcgacaggaagcagtgcgtgaagaaagagctggtgaccggcctgggactgaccggcgagctggtggacccacccttccacgagttcgcctacgagagcctgaggaccagacccgccgctccctaccaggtgcccaccatcggcgtgtacggcgtgcccggcagcggaaagagcggcatcatcaagagcgccgtgaccaagaaagacctggtggtcagcgccaagaaagagaactgcgccgagatcatcagggacgtgaagaagatgaaaggcctggacgtgaacgcgcgcaccgtggacagcgtgctgctgaacggctgcaagcaccccgtggagaccctgtacatcgacgaggccttcgcttgccacgccggcaccctgagggccctgatcgccatcatcaggcccaagaaagccgtgctgtgcggcgaccccaagcagtgcggcttcttcaacatgatgtgcctgaaggtgcacttcaaccacgagatctgcacccaggtgttccacaagagcatcagcaggcggtgcaccaagagcgtgaccagcgtcgtgagcaccctgttctacgacaagaaaatgaggaccaccaaccccaaggagaccaaaatcgtgatcgacaccacaggcagcaccaagcccaagcaggacgacctgatcctgacctgcttcaggggctgggtgaagcagctgcagatcgactacaagggcaacgagatcatgaccgccgctgccagccagggcctgaccaggaagggcgtgtacgccgtgaggtacaaggtgaacgagaacccactgtacgctcccaccagcgagcacgtgaacgtgctgctgaccaggaccgaggacaggatcgtgtggaagaccctggccggcgacccctggatcaagaccctgaccgccaagtaccccggcaacttcaccgccaccatcgaagagtggcaggccgagcacgacgccatcatgaggcacatcctggagaggcccgaccccaccgacgtgttccagaacaaggccaacgtgtgctgggccaaggccctggtgcccgtgctgaagaccgccggcatcgacatgaccacagagcagtggaacaccgtggactacttcgagaccgacaaggcccacagcgccgagatcgtgctgaaccagctgtgcgtgaggttcttcggcctggacctggacagcggcctgttcagcgcccccaccgtgccactgagcatcaggaacaaccactgggacaacagccccagcccaaacatgtacggcctgaacaaggaggtggtcaggcagctgagcaggcggtacccacagctgcccagggccgtggccaccggcagggtgtacgacatgaacaccggcaccctgaggaactacgaccccaggatcaacctggtgcccgtgaacaggcggctgccccacgccctggtgctgcaccacaacgagcacccacagagcgacttcagctccttcgtgagcaagctgaaaggcaggaccgtgctggtcgtgggcgagaagctgagcgtgcccggcaagatggtggactggctgagcgacaggcccgaggccaccttccgggccaggctggacctcggcatccccggcgacgtgcccaagtacgacatcatcttcgtgaacgtcaggaccccatacaagtaccaccattaccagcagtgcgaggaccacgccatcaagctgagcatgctgaccaagaaggcctgcctgcacctgaaccccggaggcacctgcgtgagcatcggctacggctacgccgacagggccagcgagagcatcattggcgccatcgccaggctgttcaagttcagcagggtgtgcaaacccaagagcagcctggaggaaaccgaggtgctgttcgtgttcatcggctacgaccggaaggccaggacccacaacccctacaagctgagcagcaccctgacaaacatctacaccggcagcaggctgcacgaggccggctgcgcccccagctaccacgtggtcaggggcgatatcgccaccgccaccgagggcgtgatcatcaacgctgccaacagcaagggccagcccggaggcggagtgtgcggcgccctgtacaagaagttccccgagagcttcgacctgcagcccatcgaggtgggcaaggccaggctggtgaagggcgccgctaagcacatcatccacgccgtgggccccaacttcaacaaggtgagcgaggtggaaggcgacaagcagctggccgaagcctacgagagcatcgccaagatcgtgaacgacaataactacaagagcgtggccatcccactgctcagcaccggcatcttcagcggcaacaaggacaggctgacccagagcctgaaccacctgctcaccgccctggacaccaccgatgccgacgtggccatctactgcagggacaagaagtgggagatgaccctgaaggaggccgtggccaggcgggaggccgtggaagagatctgcatcagcgacgactccagcgtgaccgagcccgacgccgagctggtgagggtgcaccccaagagctccctggccggcaggaagggctacagcaccagcgacggcaagaccttcagctacctggagggcaccaagttccaccaggccgctaaggacatcgccgagatcaacgctatgtggcccgtggccaccgaggccaacgagcaggtgtgcatgtacatcctgggcgagagcatgtccagcatcaggagcaagtgccccgtggaggaaagcgaggccagcacaccacccagcaccctgccctgcctgtgcatccacgctatgacacccgagagggtgcagcggctgaaggccagcaggcccgagcagatcaccgtgtgcagctccttcccactgcccaagtacaggatcaccggcgtgcagaagatccagtgcagccagcccatcctgttcagcccaaaggtgcccgcctacatccaccccaggaagtacctggtggagaccccacccgtggacgagacacccgagccaagcgccgagaaccagagcaccgagggcacacccgagcagccacccctgatcaccgaggacgagacaaggacccggaccccagagcccatcattatcgaggaagaggaagaggacagcatcagcctgctgagcgacggccccacccaccaggtgctgcaggtggaggccgacatccacggcccacccagcgtgtccagctccagctggagcatcccacacgccagcgacttcgacgtggacagcctgagcatcctggacaccctggagggcgccagcgtgacctccggcgccaccagcgccgagaccaacagctacttcgccaagagcatggagttcctggccaggcccgtgccagctcccaggaccgtgttcaggaacccaccccacccagctcccaggaccaggaccccaagcctggctcccagcagggcctgcagcaggaccagcctggtgagcaccccacccggcgtgaacagggtgatcaccagggaggaactggaggccctgacacccagcaggacccccagcaggtccgtgagcaggactagtctggtgtccaacccacccggcgtgaacagggtgatcaccagggaggaattcgaggccttcgtggcccagcaacagagacggttcgacgccggcgcctacatcttcagcagcgacaccggccagggacacctgcagcaaaagagcgtgaggcagaccgtgctgagcgaggtggtgctggagaggaccgagctggaaatcagctacgcccccaggctggaccaggagaaggaggaactgctcaggaagaaactgcagctgaaccccaccccagccaacaggagcaggtaccagagcaggaaggtggagaacatgaaggccatcaccgccaggcggatcctgcagggcctgggacactacctgaaggccgagggcaaggtggagtgctacaggaccctgcaccccgtgccactgtacagctccagcgtgaacagggccttctccagccccaaggtggccgtggaggcctgcaacgctatgctgaaggagaacttccccaccgtggccagctactgcatcatccccgagtacgacgcctacctggacatggtggacggcgccagctgctgcctggacaccgccagcttctgccccgccaagctgaggagcttccccaagaaacacagctacctggagcccaccatcaggagcgccgtgcccagcgccatccagaacaccctgcagaacgtgctggccgctgccaccaagaggaactgcaacgtgacccagatgagggagctgcccgtgctggacagcgctgccttcaacgtggagtgcttcaagaaatacgcctgcaacaacgagtactgggagaccttcaaggagaaccccatcaggctgaccgaagagaacgtggtgaactacatcaccaagctgaagggccccaaggccgctgccctgttcgctaagacccacaacctgaacatgctgcaggacatcccaatggacaggttcgtgatggacctgaagagggacgtgaaggtgacacccggcaccaagcacaccgaggagaggcccaaggtgcaggtgatccaggccgctgacccactggccaccgcctacctgtgcggcatccacagggagctggtgaggcggctgaacgccgtgctgctgcccaacatccacaccctgttcgacatgagcgccgaggacttcgacgccatcatcgccgagcacttccagcccggcgactgcgtgctggagaccgacatcgccagcttcgacaagagcgaggatgacgctatggccctgaccgctctgatgatcctggaggacctgggcgtggacgccgagctgctcaccctgatcgaggctgccttcggcgagatcagctccatccacctgcccaccaagaccaagttcaagttcggcgctatgatgaaaagcggaatgttcctgaccctgttcgtgaacaccgtgatcaacattgtgatcgccagcagggtgctgcgggagaggctgaccggcagcccctgcgctgccttcatcggcgacgacaacatcgtgaagggcgtgaaaagcgacaagctgatggccgacaggtgcgccacctggctgaacatggaggtgaagatcatcgacgccgtggtgggcgagaaggccccctacttctgcggcggattcatcctgtgcgacagcgtgaccggcaccgcctgcagggtggccgaccccctgaagaggctgttcaagctgggcaagccactggccgctgacgatgagcacgacgatgacaggcggagggccctgcacgaggaaagcaccaggtggaacagggtgggcatcctgagcgagctgtgcaaggccgtggagagcaggtacgagaccgtgggcaccagcatcatcgtgatggctatgaccacactggccagctccgtcaagagcttctcctacctgaggggggcccctataactctctacggctaacctgaatggactacgacatagtctagtccgccaaggccgccaccatgaaggctatcctggtggtgctgctctacacctttgccacagccaatgctgacaccctgtgtattggctaccatgccaacaacagcacagacacagtggacacagtgttggagaagaatgtgacagtgacccactctgtgaacctgttggaggacaaacacaatggcaaactgtgtaaactgaggggagtggctccactgcacctgggcaagtgtaacattgctggctggattctgggcaaccctgagtgtgagtccctgagcacagcctcctcctggtcctacattgtggagacaccatcctctgacaatggcacttgttaccctggagacttcattgactatgaggaactgagggaacaactttcctctgtgtcctcctttgagaggtttgagatttttccaaagacctcctcctggccaaaccatgacagcaacaagggagtgacagcagcctgtccacatgctggagccaagtccttctacaagaacctgatttggctggtgaagaagggcaactcctacccaaaactgagcaagtcctacatcaatgacaagggcaaggaggtgctggtgctgtggggcatccaccacccaagcacctctgctgaccaacagtccctctaccagaatgctgacgcctatgtgtttgtgggctccagcagatacagcaagaagttcaagcctgagattgccatcagaccaaaggtgagggatcaggagggcaggatgaactactactggaccctggtggaacctggagacaagattacctttgaggctacaggcaacctggtggtgccaagatatgcctttgctatggagaggaatgctggctctggcatcatcatctctgacacacctgtccatgactgtaacaccacttgtcagacaccaaagggagccatcaacacctccctgccattccagaacatccacccaatcaccattggcaagtgtccaaaatatgtcaagagcaccaaactgagactggctacaggactgaggaacatcccaagcatccagagcaggggactgtttggagccattgctggcttcattgagggaggctggacagggatggtggatggctggtatggctaccaccaccagaatgaacagggctctggctatgctgctgacctgaaaagcacccagaatgccattgatgagattaccaacaaggtgaactctgtgattgagaagatgaacacccagttcacagcagtgggcaaggagttcaaccacttggagaagaggattgagaacctgaacaagaaggtggatgatggcttcctggacatctggacctacaatgctgaactgctggtgctgttggagaatgagaggaccctggactaccatgacagcaatgtgaagaacctctatgagaaggtgaggagccaacttaaaaacaatgccaaggagattggcaatggctgttttgagttctaccacaagtgtgacaacacttgtatggagtctgtgaagaatggcacctatgactacccaaaatactctgaggaggctaaactgaacagggaggagattgatggagtgaaattggagagcaccaggatttaccagatcctggccatctacagcaccgtggccagcagcctggtgctggtggtgagcctgggcgccatcagcttctggatgtgcagcaacggcagcttgcagtgcaggatctgcatctaaactcgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaatctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 57 - RNA, 448 nt, synthetic construct
  misc_feature 1..448: note = Synthetic polynucleotide
  source 1..448: mol_type = other RNA
gataggcggcgcatgagagaagcccagaccaattacctacccaaataggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaatagtcagcatagtacatttcatctgactaatactacaacaccaccaccatgaatagaggattctttaacatgctcggccgccgccccttcccggcccccactgccatgtggaggccgcggagaaggaggcaggcggccccgggaagcggagctactaacttcagcctgctgaagcaggctggagacgtggaggagaaccctggacct

SEQ ID NO: 58-66 - skipped (9 entries)

SEQ ID NO: 67 - DNA, 30 bp, synthetic construct
  misc_feature 1..30: note = Synthetic polynucleotide
  source 1..30: mol_type = other DNA
atggactacgacatagtctagtccgccaag

SEQ ID NO: 68 - DNA, 16 bp, synthetic construct
  misc_feature 1..16: note = Synthetic polynucleotide
  source 1..16: mol_type = other DNA
atggactacgacatag

SEQ ID NO: 69 - DNA, 15 bp, synthetic construct
  misc_feature 1..15: note = Synthetic polynucleotide
  source 1..15: mol_type = other DNA
atggactacgacata

SEQ ID NO: 70-71 - skipped (2 entries)

SEQ ID NO: 72 - DNA, 7482 bp, synthetic construct; identical to SEQ ID NO: 50
  misc_feature 1..7482: note = Synthetic polynucleotide
  source 1..7482: mol_type = other DNA

SEQ ID NO: 73 - DNA, 44 bp, synthetic construct; identical to SEQ ID NO: 49
  misc_feature 1..44: note = Synthetic polynucleotide
  source 1..44: mol_type = other DNA

SEQ ID NO: 74 - DNA, 45 bp, synthetic construct
  misc_feature 1..45: note = Synthetic polynucleotide
  source 1..45: mol_type = other DNA
gatgggcggcgcatgagagaagcccagaccaattacctacccaaa

SEQ ID NO: 75 - DNA, 45 bp, synthetic construct
  misc_feature 1..45: note = Synthetic polynucleotide
  source 1..45: mol_type = other DNA
gataggcggcgcatgagagaagcccagaccaattacctacccaaa

SEQ ID NO: 76 - DNA, 468 bp, synthetic construct; identical to SEQ ID NO: 53
  misc_feature 1..468: note = Synthetic polynucleotide
  source 1..468: mol_type = other DNA

SEQ ID NO: 77 - DNA, 44 bp, synthetic construct; identical to SEQ ID NO: 52
  misc_feature 1..44: note = Synthetic polynucleotide
  source 1..44: mol_type = other DNA

SEQ ID NO: 78 - DNA, 8038 bp, synthetic construct
  misc_feature 1..8038: note = Synthetic polynucleotide
  source 1..8038: mol_type = other DNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgccgtcgacggccccaccagcctgtaccaccaggccaacaagggcgtgagggtggcctactggatcggcttcgacaccacacccttcatgttcaagaacctggccggcgcctaccccagctacagcaccaactgggccgacgagaccgtgctgaccgccaggaacatcggcctgtgcagcagcgacgtgatggagaggagccggagaggcatgagcatcctgaggaagaaatacctgaagcccagcaacaacgtgctgttcagcgtgggcagcaccatctaccacgagaagagggacctgctcaggagctggcacctgcccagcgtgttccacctgaggggcaagcagaactacacctgcaggtgcgagaccatcgtgagctgcgacggctacgtggtgaagaggatcgccatcagccccggcctgtacggcaagcccagcggctacgccgctacaatgcacagggagggcttcctgtgctgcaaggtgaccgacaccctgaacggcgagagggtgagcttccccgtgtgcacctacgtgcccgccaccctgtgcgaccagatgaccggcatcctggccaccgacgtgagcgccgacgacgcccagaagctgctcgtgggcctgaaccagaggatcgtggtcaacggcaggacccagaggaacaccaacacaatgaagaactacctgctgcccgtggtggcccaggctttcgccaggtgggccaaggagtacaaggaggaccaggaagacgagaggcccctgggcctgagggacaggcagctggtgatgggctgctgctgggccttcaggcggcacaagatcaccagcatctacaagaggcccgacacccagaccatcatcaaggtgaacagcgacttccacagcttcgtgctgcccaggatcggcagcaacaccctggagatcggcctgaggacccggatcaggaagatgctggaggaacacaaggagcccagcccactgatcaccgccgaggacgtgcaggaggccaagtgcgctgccgacgaggccaaggaggtgagggaggccgaggaactgagggccgccctgccacccctggctgccgacgtggaggaacccaccctggaagccgacgtggacctgatgctgcaggaggccggcgccggaagcgtggagacacccaggggcctgatcaaggtgaccagctacgacggcgaggacaagatcggcagctacgccgtgctgagcccacaggccgtgctgaagtccgagaagctgagctgcatccacccactggccgagcaggtgatcgtgatcacccacagcggcaggaagggcaggtacgccgtggagccctaccacggcaaggtggtcgtgcccgagggccacgccatccccgtgcaggacttccaggccctgagcgagagcgccaccatcgtgtacaacgagagggagttcgtgaacaggtacctgcaccatatcgccacccacggcggagccctgaacaccgacgaggaatactacaagaccgtgaagcccagcgagcacgacggcgagtacctgtacgacatcgacaggaagcagtgcgtgaagaaagagctggtgaccggcctgggactgaccggcgagctggtggacccacccttccacgagttcgcctacgagagcctgaggaccagacccgccgctccctaccaggtgcccaccatcggcgtgtacggcgtgcccggcagcggaaagagcggcatcatcaagagcgccgtgaccaagaaagacctggtggtcagcgccaagaaagagaactgcgccgagatcatcagggacgtgaagaagatgaaaggcctggacgtgaacgcgcgcaccgtggacagcgtgctgctgaacggctgcaagcaccccgtggagaccctgtacatcgacgaggccttcgcttgccacgccggcaccctgagggccctgatcgccatcatcaggcccaagaaagccgtgctgtgcggcgaccccaagcagtgcggcttcttcaacatgatgtgcctgaaggtgcacttcaaccacgagatctgcacccaggtgttccacaagagcatcagcaggcggtgcaccaagagcgtgaccagcgtcgtgagcaccctgttctacgacaagaaaatgaggaccaccaaccccaaggagaccaaaatcgtgatcgacaccacaggcagcaccaagcccaagcaggacgacctgatcctgacctgcttcaggggctgggtgaagcagctgcagatcgactacaagggcaacgagatcatgaccgccgctgccagccagggcctgaccaggaagggcgtgtacgccgtgaggtacaaggtgaacgagaacccactgtacgctcccaccagcgagcacgtgaacgtgctgctgaccaggaccgaggacaggatcgtgtggaagaccctggccggcgacccctggatcaagaccctgaccgccaagtaccccggcaacttcaccgccaccatcgaagagtggcaggccgagcacgacgccatcatgaggcacatcctggagaggcccgaccccaccgacgtgttccagaacaaggccaacgtgtgctgggccaaggccctggtgcccgtgctgaagaccgccggcatcgacatgaccacagagcagtggaacaccgtggactacttcgagaccgacaaggcccacagcgccgagatcgtgctgaaccagctgtgcgtgaggttcttcggcctggacctggacagcggcctgttcagcgcccccaccgtgccactgagcatcaggaacaaccactgggacaacagccccagcccaaacatgtacggcctgaacaaggaggtggtcaggcagctgagcaggcggtacccacagctgcccagggccgtggccaccggcagggtgtacgacatgaacaccggcaccctgaggaactacgaccccaggatcaacctggtgcccgtgaacaggcggctgccccacgccctggtgctgcaccacaacgagcacccacagagcgacttcagctccttcgtgagcaagctgaaaggcaggaccgtgctggtcgtgggcgagaagctgagcgtgcccggcaagatggtggactggctgagcgacaggcccgaggccaccttccgggccaggctggacctcggcatccccggcgacgtgcccaagtacgacatcatcttcgtgaacgtcaggaccccatacaagtaccaccattaccagcagtgcgaggaccacgccatcaagctgagcatgctgaccaagaaggcctgcctgcacctgaaccccggaggcacctgcgtgagcatcggctacggctacgccgacagggccagcgagagcatcattggcgccatcgccaggctgttcaagttcagcagggtgtgcaaacccaagagcagcctggaggaaaccgaggtgctgttcgtgttcatcggctacgaccggaaggccaggacccacaacccctacaagctgagcagcaccctgacaaacatctacaccggcagcaggctgcacgaggccggctgcgcccccagctaccacgtggtcaggggcgatatcgccaccgccaccgagggcgtgatcatcaacgctgccaacagcaagggccagcccggaggcggagtgtgcggcgccctgtacaagaagttccccgagagcttcgacctgcagcccatcgaggtgggcaaggccaggctggtgaagggcgccgctaagcacatcatccacgccgtgggccccaacttcaacaaggtgagcgaggtggaaggcgacaagcagctggccgaagcctacgagagcatcgccaagatcgtgaacgacaataactacaagagcgtggccatcccactgctcagcaccggcatcttcagcggcaacaaggacaggctgacccagagcctgaaccacctgctcaccgccctggacaccaccgatgccgacgtggccatctactgcagggacaagaagtgggagatgaccctgaaggaggccgtggccaggcgggaggccgtggaagagatctgcatcagcgacgactccagcgtgaccgagcccgacgccgagctggtgagggtgcaccccaagagctccctggccggcaggaagggctacagcaccagcgacggcaagaccttcagctacctggagggcaccaagttccaccaggccgctaaggacatcgccgagatcaacgctatgtggcccgtggccaccgaggccaacgagcaggtgtgcatgtacatcctgggcgagagcatgtccagcatcaggagcaagtgccccgtggaggaaagcgaggccagcacaccacccagcaccctgccctgcctgtgcatccacgctatgacacccgagagggtgcagcggctgaaggccagcaggcccgagcagatcaccgtgtgcagctccttcccactgcccaagtacaggatcaccggcgtgcagaagatccagtgcagccagcccatcctgttcagcccaaaggtgcccgcctacatccaccccaggaagtacctggtggagaccccacccgtggacgagacacccgagccaagcgccgagaaccagagcaccgagggcacacccgagcagccacccctgatcaccgaggacgagacaaggacccggaccccagagcccatcattatcgaggaagaggaagaggacagcatcagcctgctgagcgacggccccacccaccaggtgctgcaggtggaggccgacatccacggcccacccagcgtgtccagctccagctggagcatcccacacgccagcgacttcgacgtggacagcctgagcatcctggacaccctggagggcgccagcgtgacctccggcgccaccagcgccgagaccaacagctacttcgccaagagcatggagttcctggccaggcccgtgccagctcccaggaccgtgttcaggaacccaccccacccagctcccaggaccaggaccccaagcctggctcccagcagggcctgcagcaggaccagcctggtgagcaccccacccggcgtgaacagggtgatcaccagggaggaactggaggccctgacacccagcaggacccccagcaggtccgtgagcaggactagtctggtgtccaacccacccggcgtgaacagggtgatcaccagggaggaattcgaggccttcgtggcccagcaacagagacggttcgacgccggcgcctacatcttcagcagcgacaccggccagggacacctgcagcaaaagagcgtgaggcagaccgtgctgagcgaggtggtgctggagaggaccgagctggaaatcagctacgcccccaggctggaccaggagaaggaggaactgctcaggaagaaactgcagctgaaccccaccccagccaacaggagcaggtaccagagcaggaaggtggagaacatgaaggccatcaccgccaggcggatcctgcagggcctgggacactacctgaaggccgagggcaaggtggagtgctacaggaccctgcaccccgtgccactgtacagctccagcgtgaacagggccttctccagccccaaggtggccgtggaggcctgcaacgctatgctgaaggagaacttccccaccgtggccagctactgcatcatccccgagtacgacgcctacctggacatggtggacggcgccagctgctgcctggacaccgccagcttctgccccgccaagctgaggagcttccccaagaaacacagctacctggagcccaccatcaggagcgccgtgcccagcgccatccagaacaccctgcagaacgtgctggccgctgccaccaagaggaactgcaacgtgacccagatgagggagctgcccgtgctggacagcgctgccttcaacgtggagtgcttcaagaaatacgcctgcaacaacgagtactgggagaccttcaaggagaaccccatcaggctgaccgaagagaacgtggtgaactacatcaccaagctgaagggccccaaggccgctgccctgttcgctaagacccacaacctgaacatgctgcaggacatcccaatggacaggttcgtgatggacctgaagagggacgtgaaggtgacacccggcaccaagcacaccgaggagaggcccaaggtgcaggtgatccaggccgctgacccactggccaccgcctacctgtgcggcatccacagggagctggtgaggcggctgaacgccgtgctgctgcccaacatccacaccctgttcgacatgagcgccgaggacttcgacgccatcatcgccgagcacttccagcccggcgactgcgtgctggagaccgacatcgccagcttcgacaagagcgaggatgacgctatggccctgaccgctctgatgatcctggaggacctgggcgtggacgccgagctgctcaccctgatcgaggctgccttcggcgagatcagctccatccacctgcccaccaagaccaagttcaagttcggcgctatgatgaaaagcggaatgttcctgaccctgttcgtgaacaccgtgatcaacattgtgatcgccagcagggtgctgcgggagaggctgaccggcagcccctgcgctgccttcatcggcgacgacaacatcgtgaagggcgtgaaaagcgacaagctgatggccgacaggtgcgccacctggctgaacatggaggtgaagatcatcgacgccgtggtgggcgagaaggccccctacttctgcggcggattcatcctgtgcgacagcgtgaccggcaccgcctgcagggtggccgaccccctgaagaggctgttcaagctgggcaagccactggccgctgacgatgagcacgacgatgacaggcggagggccctgcacgaggaaagcaccaggtggaacagggtgggcatcctgagcgagctgtgcaaggccgtggagagcaggtacgagaccgtgggcaccagcatcatcgtgatggctatgaccacactggccagctccgtcaagagcttctcctacctgaggggggcccctataactctctacggctaacctgaatggactacgacatagtctagtccgccaaggccgccaccactcgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaatctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 79 - protein, 2493 aa, synthetic construct; identical to SEQ ID NO: 51
  REGION 1..2493: note = Synthetic polypeptide

SEQ ID NO: 80 - protein, 2494 aa, synthetic construct; identical to SEQ ID NO: 55
  REGION 1..2494: note = Synthetic polypeptide

SEQ ID NO: 81 - protein, 2492 aa, synthetic construct
  REGION 1..2492: note = Synthetic polypeptide
MEKVHVDIEEDSPFLRALQRSFPQFEVEAKQVTDNDHANARAFSHLASKLIETEVDPSDTILDIGSAPARRMYSKHKYHCICPMRCAEDPDRLYKYATKLKKNCKEITDKELDKKMKELAAVMSDPDLETETMCLHDDESCRYEGQVAVYQDVYAVDGPTSLYHQANKGVRVAYWIGFDTTPFMFKNLAGAYPSYSTNWADETVLTARNIGLCSSDVMERSRRGMSILRKKYLKPSNNVLFSVGSTIYHEKRDLLRSWHLPSVFHLRGKQNYTCRCETIVSCDGYVVKRIAISPGLYGKPSGYAATMHREGFLCCKVTDTLNGERVSFPVCTYVPATLCDQMTGILATDVSADDAQKLLVGLNQRIVVNGRTQRNTNTMKNYLLPVVAQAFARWAKEYKEDQEDERPLGLRDRQLVMGCCWAFRRHKITSIYKRPDTQTIIKVNSDFHSFVLPRIGSNTLEIGLRTRIRKMLEEHKEPSPLITAEDIQEAKCAADEAKEVREAEELRAALPPLAADFEEPTLEADVDLMLQEAGAGSVETPRGLIKVTSYAGEDKIGSYAVLSPQAVLKSEKLSCIHPLAEQVIVITHSGRKGRYAVEPYHGKVVVPEGHAIPVQDFQALSESATIVYNEREFVNRYLHHIATHGGALNTDEEYYKTVKPSEHDGEYLYDIDRKQCVKKELVTGLGLTGELVDPPFHEFAYESLRTRPAAPYQVPTIGVYGVPGSGKSGIIKSAVTKKDLVVSAKKENCAEIIRDVKKMKGLDVNARTVDSVLLNGCKHPVETLYIDEAFACHAGTLRALIAIIRPKKAVLCGDPKQCGFFNMMCLKVHFNHEICTQVFHKSISRRCTKSVTSVVSTLFYDKRMRTTNPKETKIVIDTTGSTKPKQDDLILTCFRGWVKQLQIDYKGNEIMTAAASQGLTRKGVYAVRYKVNENPLYAPTSEHVNVLLTRTEDRIVWKTLAGDPWIKILTAKYPGNFTATIEEWQAEHDAIMRHILERPDPTDVFQNKANVCWAKALVPVLKTAGIDMTTEQWNTVDYFETDKAHSAEIVLNQLCVRFFGLDLDSGLFSAPTVPLSIRNNHWDNSPSPNMYGLNKEVVRQLSRRYPQLPRAVATGRVYDMNTGTLRNYDPRINLVPVNRRLPHALVLHHNEHPQSDFSSFVSKLKGRTVLVVGEKLSVPGKKVDWLSDQPEATFRARLDLGIPGDVPKYDIVFINVRTPYKYHHYQQCEDHAIKLSMLTKKACLHLNPGGTCVSIGYGYADRASESIIGAIARQFKFSRVCKPKSSHEETEVLFVFIGYDRKARTHNPYKLSSTLTNIYTGSRLHEAGCAPSYHVVRGDIATATEGVIINAANSKGQPGGGVCGALYKKFPESFDLQPIEVGKARLVKGAAKHIIHAVGPNFNKVSEVEGDKQLAEAYESIAKIVNDNNYKSVAIPLLSTGIFSGNKDRLTQSLNHLLTALDTTDADVAIYCRDKKWEMTLKEAVARREAVEEICISDDSSVTEPDAELVRVHPKSSLAGRKGYSTSDGKTFSYLEGTKFHQAAKDIAEINAMWPVATEANEQVCMYILGESMSSIRSKCPVEESEASTPPSTLPCLCIHAMTPERVQRLKASRPEQITVCSSFPLPKYRITGVQKIQCSQPILFSPKVPAYIHPRKYLVETPPVEETPESPAENQSTEGTPEQPALVNVDATRTRMPEPIIIEEEEEDSISLLSDGPTHQVLQVEADIHGSPSVSSSSWSIPHASDFDVDSLSILDTLDGASVTSGAVSAETNSYFARSMEFRARPVPAPRTVFRNPPHPAPRTRTPPLAHSRASSRTSLVSTPPGVNRVITREELEALTPSRAPSRSASRTSLVSNPPGVNRVITREEFEAFVAQQQRFDAGAYIFSSDTGQGHLQQKSVRQTVLSEVVLERTELEISYAPRLDQEKEELLRKKLQLNPTPANRSRYQSRRVENMKAITARRILQGLGHYLKAEGKVECYRTLHPVPLYSSSVNRAFSSPKVAVEACNAMLKENFPTVASYCIIPEYDAYLDMVDGASCCLDTASFCPAKLRSFPKKHSYLEPTIRSAVPSAIQNTLQNVLAAATKRNCNVTQMRELPVLDSAAFNVECFKKYACNNEYWETFKENPIRLTEENVVNYITKLKGPKAAALFAKTHNLNMLQDIPMDRFVMDLKRDVKVTPGTKHTEERPKVQVIQAADPLATADLCGIHRELVRRLNAVLLPNIHTLFDMSAEDFDAIIAEHFQPGDCVLETDIASFDKSEDDAMALTALMILEDLGVDAELLTLIEAAFGEISSIHLPTKTKFKFGAMMKSGMFLTLFVNTVINIVIASRVLRERLTGSPCAAFIGDDNIVKGVKSDKLMADRCATWLNMEVKIIDAVVGEKAPYFCGGFILCDSVTGTACRVADPLKRLFKLGKPLAVDDEHDDDRRRALHEESTRWNRVGILPELCKAVESRYETVGTSIIVMAMTTLASSVKSFSYLRGAPITLYG

SEQ ID NO: 82 - DNA, 146 bp, synthetic construct
  misc_feature 1..146: note = Synthetic polynucleotide
  source 1..146: mol_type = other DNA
aggaaacttaagtcaacacaacatatacaaaacaaacgaatctcaagcaatcaagcattctacttctattgcagcaatttaaatcatttcttttaaagcaaaagcaattttctgaaaattttcaccatttacgaacgatagccacc

SEQ ID NO: 83 - DNA, 270 bp, synthetic construct
  misc_feature 1..270: note = Synthetic polynucleotide
  source 1..270: mol_type = other DNA
actcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 84 - RNA, 1653 nt, synthetic construct
  misc_feature 1..1653: note = Synthetic polynucleotide
  source 1..1653: mol_type = other RNA
atggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgacagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagtccctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaa

SEQ ID NO: 85 - RNA, 1653 nt, synthetic construct; identical to SEQ ID NO: 84
  misc_feature 1..1653: note = Synthetic polynucleotide
  source 1..1653: mol_type = other RNA

SEQ ID NO: 86 - RNA, 1653 nt, synthetic construct; identical to SEQ ID NO: 84
  misc_feature 1..1653: note = Synthetic polynucleotide
  source 1..1653: mol_type = other RNA

SEQ ID NO: 87 - RNA, 57 nt, synthetic construct
  misc_feature 1..57: note = Synthetic polynucleotide
  source 1..57: mol_type = other RNA
atgaagttggtggttgtgggggccgggggtgttggcaaaagcgcccttacaatttga

SEQ ID NO: 88 - RNA, 66 nt, synthetic construct
  misc_feature 1..66: note = Synthetic polynucleotide
  source 1..66: mol_type = other RNA
atggatcctagacgctacgccccaatgatccgaccagcaaaactcgatgtacttccgaggaactga

SEQ ID NO: 89 - RNA, 837 nt, synthetic construct
  misc_feature 1..837: note = Synthetic polynucleotide
  source 1..837: mol_type = other RNA
atgagagtgacagcccctagaaccttactgcttctgctttggggagctgttgctctgacagagacatgggctggatctctgagcgaggtgaccggccagggcctgtgcatcggcgccgtgcccaagacccaccaggtgctgtgcaacaccacccagaagaccagcgacggcagctactacctggccgctcccaccggcaccacctgggcctgcagcaccggcctgaccccttgcatcagcaccaccatcctgaacctgaccaccgactactgcgtgctggtggagctgtggcccagggtgacctaccacagccccagctacgcctaccaccagttcgagaggagggccaagtacaagagggagcccgtgagcctgaccctggccctgctgctgggcggcctgacaatgggcggcatcgccgccggcgtgggcaccggcaccaccgccctggtggccacccagcagttccagcagctgcaggccgccatgcacgacgacctgaaggaggtggagaagtccatcaccaacctggagaagtccctgaccagcctgagcgaggtggtgctgcagaacaggaggggcctggacctgctgttcctgaaggagggcggcctgtgcgccgccctgaaggaggagtgctgcctgtacgccgaccacaccggcctggtgatcgtgggcattgtcgctggcctggccgtcctcgccgtggtggtgattggagctgtggtcgcagctgttatgtgcagaagaaagtcatccggcggaaagggaggctcctactctcaggctgcttctgctacagtgcctagagctcttatgtgtttatctcagctgtaa

SEQ ID NO: 90 - RNA, 378 nt, synthetic construct
  misc_feature 1..378: note = Synthetic polynucleotide
  source 1..378: mol_type = other RNA
atgagagtgacagcccctagaaccttactgcttctgctttggggagctgttgctctgacagagacatgggctggatcttaccacagccccagctacgcctaccaccagttcgagagggggggaggaggctccgggggaggaggctccctgaagatcagccaggccgtgcacgccgcccacgccgagatcaacgaggccggccgggaggtgatcgtgggcattgtcgctggcctggccgtcctcgccgtggtggtgattggagctgtggtcgcagctgttatgtgcagaagaaagtcatccggcggaaagggaggctcctactctcaggctgcttctgctacagtgcctagagctcttatgtgtttatctcagctgtaa

SEQ ID NO: 91 - RNA, 876 nt, synthetic construct
  misc_feature 1..876: note = Synthetic polynucleotide
  source 1..876: mol_type = other RNA
atgagagtgacagcccctagaaccttactgcttctgctttggggagctgttgctctgacagagacatgggctggatctctgagcgaggtgaccggccagggcctgtgcatcggcgccgtgcccaagacccaccaggtgctgtgcaacaccacccagaagaccagcgacggcagctactacctggccgctcccaccggcaccacctgggcctgcagcaccggcctgaccccttgcatcagcaccaccatcctgaacctgaccaccgactactgcgtgctggtggagctgtggcccagggtgacctaccacagccccagctacgcctaccaccagttcgagaggagggccaagtacaagagggagcccgtgagcctgaccctggccctgctgctgggcggcctgacaatgggcggcatcgccgccggcgtgggcaccggcaccaccgccctggtggccacccagcagttccagcagctgcaggccgccatgcacgacgacctgaaggaggtggagaagtccatcaccaacctggagaagtccctgaccagcctgagcgaggtggtgctgcagaacaggaggggcctggacctgctgttcctgaaggagggcggcctgtgcgccgccctgaaggaggagtgctgcctgtacgccgaccacaccggcctggtgatcgtgggcattgtcgctggcctggccgtcctcgccgtggtggtgattggagctgtggtcgcagctgttatgtgcagaagaaagtcatccggcggaaagggaggctcctactctcaggctgcttctgctacagtgcctagagctcttatgtgtttatctcagctgggcggcggaggcagcgactacaaggacgacgatgacaagtaa

SEQ ID NO: 92 - RNA, 417 nt, synthetic construct
  misc_feature 1..417: note = Synthetic polynucleotide
  source 1..417: mol_type = other RNA
atgagagtgacagcccctagaaccttactgcttctgctttggggagctgttgctctgacagagacatgggctggatcttaccacagccccagctacgcctaccaccagttcgagagggggggaggaggctccgggggaggaggctccctgaagatcagccaggccgtgcacgccgcccacgccgagatcaacgaggccggccgggaggtgatcgtgggcattgtcgctggcctggccgtcctcgccgtggtggtgattggagctgtggtcgcagctgttatgtgcagaagaaagtcatccggcggaaagggaggctcctactctcaggctgcttctgctacagtgcctagagctcttatgtgtttatctcagctgggcggcggaggcagcgactacaaggacgacgatgacaagtaa

SEQ ID NO: 93 - protein, 550 aa, synthetic construct
  REGION 1..550: note = Synthetic polypeptide
MEDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIAV

SEQ ID NO: 94 - protein, 18 aa, synthetic construct
  REGION 1..18: note = Synthetic polypeptide
MKLVVVGAGGVGKSALTI

SEQ ID NO: 95 - protein, 21 aa, synthetic construct
  REGION 1..21: note = Synthetic polypeptide
MDPRRYAPMIRPAKLDVLPRN

SEQ ID NO: 96 - protein, 278 aa, synthetic construct
  REGION 1..278: note = Synthetic polypeptide
MRVTAPRTLLLLLWGAVALTETWAGSLSEVTGQGLCIGAVPKTHQVLCNTTQKTSDGSYYLAAPTGTTWACSTGLTPCISTTILNLTTDYCVLVELWPRVTYHSPSYAYHQFERRAKYKREPVSLTLALLLGGLTMGGIAAGVGTGTTALVATQQFQQLQAAMHDDLKEVEKSITNLEKSLTSLSEVVLQNRRGLDLLFLKEGGLCAALKEECCLYADHTGLVIVGIVAGLAVLAVVVIGAVVAAVMCRRKSSGGKGGSYSQAASATVPRALMCLSQL

SEQ ID NO: 97 - protein, 125 aa, synthetic construct
  REGION 1..125: note = Synthetic polypeptide
MRVTAPRTLLLLLWGAVALTETWAGSYHSPSYAYHQFERGGGGSGGGGSLKISQAVHAAHAEINEAGREVIVGIVAGLAVLAVVVIGAVVAAVMCRRKSSGGKGGSYSQAASATVPRALMCLSQL

SEQ ID NO: 98 - protein, 291 aa, synthetic construct
  REGION 1..291: note = Synthetic polypeptide
MRVTAPRTLLLLLWGAVALTETWAGSLSEVTGQGLCIGAVPKTHQVLCNTTQKTSDGSYYLAAPTGTTWACSTGLTPCISTTILNLTTDYCVLVELWPRVTYHSPSYAYHQFERRAKYKREPVSLTLALLLGGLTMGGIAAGVGTGTTALVATQQFQQLQAAMHDDLKEVEKSITNLEKSLTSLSEVVLQNRRGLDLLFLKEGGLCAALKEECCLYADHTGLVIVGIVAGLAVLAVVVIGAVVAAVMCRRKSSGGKGGSYSQAASATVPRALMCLSQLGGGGSDYKDDDDK

SEQ ID NO: 99 - protein, 138 aa, synthetic construct
  REGION 1..138: note = Synthetic polypeptide
MRVTAPRTLLLLLWGAVALTETWAGSYHSPSYAYHQFERGGGGSGGGGSLKISQAVHAAHAEINEAGREVIVGIVAGLAVLAVVVIGAVVAAVMCRRKSSGGKGGSYSQAASATVPRALMCLSQLGGGGSDYKDDDDK

SEQ ID NO: 100 - RNA, 9690 nt, synthetic construct
  misc_feature 1..9690: note = Synthetic polynucleotide
  source 1..9690: mol_type = other RNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgccgtcgacggccccaccagcctgtaccaccaggccaacaagggcgtgagggtggcctactggatcggcttcgacaccacacccttcatgttcaagaacctggccggcgcctaccccagctacagcaccaactgggccgacgagaccgtgctgaccgccaggaacatcggcctgtgcagcagcgacgtgatggagaggagccggagaggcatgagcatcctgaggaagaaatacctgaagcccagcaacaacgtgctgttcagcgtgggcagcaccatctaccacgagaagagggacctgctcaggagctggcacctgcccagcgtgttccacctgaggggcaagcagaactacacctgcaggtgcgagaccatcgtgagctgcgacggctacgtggtgaagaggatcgccatcagccccggcctgtacggcaagcccagcggctacgccgctacaatgcacagggagggcttcctgtgctgcaaggtgaccgacaccctgaacggcgagagggtgagcttccccgtgtgcacctacgtgcccgccaccctgtgcgaccagatgaccggcatcctggccaccgacgtgagcgccgacgacgcccagaagctgctcgtgggcctgaaccagaggatcgtggtcaacggcaggacccagaggaacaccaacacaatgaagaactacctgctgcccgtggtggcccaggctttcgccaggtgggccaaggagtacaaggaggaccaggaagacgagaggcccctgggcctgagggacaggcagctggtgatgggctgctgctgggccttcaggcggcacaagatcaccagcatctacaagaggcccgacacccagaccatcatcaaggtgaacagcgacttccacagcttcgtgctgcccaggatcggcagcaacaccctggagatcggcctgaggacccggatcaggaagatgctggaggaacacaaggagcccagcccactgatcaccgccgaggacgtgcaggaggccaagtgcgctgccgacgaggccaaggaggtgagggaggccgaggaactgagggccgccctgccacccctggctgccgacgtggaggaacccaccctggaagccgacgtggacctgatgctgcaggaggccggcgccggaagcgtggagacacccaggggcctgatcaaggtgaccagctacgacggcgaggacaagatcggcagctacgccgtgctgagcccacaggccgtgctgaagtccgagaagctgagctgcatccacccactggccgagcaggtgatcgtgatcacccacagcggcaggaagggcaggtacgccgtggagccctaccacggcaaggtggtcgtgcccgagggccacgccatccccgtgcaggacttccaggccctgagcgagagcgccaccatcgtgtacaacgagagggagttcgtgaacaggtacctgcaccatatcgccacccacggcggagccctgaacaccgacgaggaatactacaagaccgtgaagcccagcgagcacgacggcgagtacctgtacgacatcgacaggaagcagtgcgtgaagaaagagctggtgaccggcctgggactgaccggcgagctggtggacccacccttccacgagttcgcctacgagagcctgaggaccagacccgccgctccctaccaggtgcccaccatcggcgtgtacggcgtgcccggcagcggaaagagcggcatcatcaagagcgccgtgaccaagaaagacctggtggtcagcgccaagaaagagaactgcgccgagatcatcagggacgtgaagaagatgaaaggcctggacgtgaacgcgcgcaccgtggacagcgtgctgctgaacggctgcaagcaccccgtggagaccctgtacatcgacgaggccttcgcttgccacgccggcaccctgagggccctgatcgccatcatcaggcccaagaaagccgtgctgtgcggcgaccccaagcagtgcggcttcttcaacatgatgtgcctgaaggtgcacttcaaccacgagatctgcacccaggtgttccacaagagcatcagcaggcggtgcaccaagagcgtgaccagcgtcgtgagcaccctgttctacgacaagaaaatgaggaccaccaaccccaaggagaccaaaatcgtgatcgacaccacaggcagcaccaagcccaagcaggacgacctgatcctgacctgcttcaggggctgggtgaagcagctgcagatcgactacaagggcaacgagatcatgaccgccgctgccagccagggcctgaccaggaagggcgtgtacgccgtgaggtacaaggtgaacgagaacccactgtacgctcccaccagcgagcacgtgaacgtgctgctgaccaggaccgaggacaggatcgtgtggaagaccctggccggcgacccctggatcaagaccctgaccgccaagtaccccggcaacttcaccgccaccatcgaagagtggcaggccgagcacgacgccatcatgaggcacatcctggagaggcccgaccccaccgacgtgttccagaacaaggccaacgtgtgctgggccaaggccctggtgcccgtgctgaagaccgccggcatcgacatgaccacagagcagtggaacaccgtggactacttcgagaccgacaaggcccacagcgccgagatcgtgctgaaccagctgtgcgtgaggttcttcggcctggacctggacagcggcctgttcagcgcccccaccgtgccactgagcatcaggaacaaccactgggacaacagccccagcccaaacatgtacggcctgaacaaggaggtggtcaggcagctgagcaggcggtacccacagctgcccagggccgtggccaccggcagggtgtacgacatgaacaccggcaccctgaggaactacgaccccaggatcaacctggtgcccgtgaacaggcggctgccccacgccctggtgctgcaccacaacgagcacccacagagcgacttcagctccttcgtgagcaagctgaaaggcaggaccgtgctggtcgtgggcgagaagctgagcgtgcccggcaagatggtggactggctgagcgacaggcccgaggccaccttccgggccaggctggacctcggcatccccggcgacgtgcccaagtacgacatcatcttcgtgaacgtcaggaccccatacaagtaccaccattaccagcagtgcgaggaccacgccatcaagctgagcatgctgaccaagaaggcctgcctgcacctgaaccccggaggcacctgcgtgagcatcggctacggctacgccgacagggccagcgagagcatcattggcgccatcgccaggctgttcaagttcagcagggtgtgcaaacccaagagcagcctggaggaaaccgaggtgctgttcgtgttcatcggctacgaccggaaggccaggacccacaacccctacaagctgagcagcaccctgacaaacatctacaccggcagcaggctgcacgaggccggctgcgcccccagctaccacgtggtcaggggcgatatcgccaccgccaccgagggcgtgatcatcaacgctgccaacagcaagggccagcccggaggcggagtgtgcggcgccctgtacaagaagttccccgagagcttcgacctgcagcccatcgaggtgggcaaggccaggctggtgaagggcgccgctaagcacatcatccacgccgtgggccccaacttcaacaaggtgagcgaggtggaaggcgacaagcagctggccgaagcctacgagagcatcgccaagatcgtgaacgacaataactacaagagcgtggccatcccactgctcagcaccggcatcttcagcggcaacaaggacaggctgacccagagcctgaaccacctgctcaccgccctggacaccaccgatgccgacgtggccatctactgcagggacaagaagtgggagatgaccctgaaggaggccgtggccaggcgggaggccgtggaagagatctgcatcagcgacgactccagcgtgaccgagcccgacgccgagctggtgagggtgcaccccaagagctccctggccggcaggaagggctacagcaccagcgacggcaagaccttcagctacctggagggcaccaagttccaccaggccgctaaggacatcgccgagatcaacgctatgtggcccgtggccaccgaggccaacgagcaggtgtgcatgtacatcctgggcgagagcatgtccagcatcaggagcaagtgccccgtggaggaaagcgaggccagcacaccacccagcaccctgccctgcctgtgcatccacgctatgacacccgagagggtgcagcggctgaaggccagcaggcccgagcagatcaccgtgtgcagctccttcccactgcccaagtacaggatcaccggcgtgcagaagatccagtgcagccagcccatcctgttcagcccaaaggtgcccgcctacatccaccccaggaagtacctggtggagaccccacccgtggacgagacacccgagccaagcgccgagaaccagagcaccgagggcacacccgagcagccacccctgatcaccgaggacgagacaaggacccggaccccagagcccatcattatcgaggaagaggaagaggacagcatcagcctgctgagcgacggccccacccaccaggtgctgcaggtggaggccgacatccacggcccacccagcgtgtccagctccagctggagcatcccacacgccagcgacttcgacgtggacagcctgagcatcctggacaccctggagggcgccagcgtgacctccggcgccaccagcgccgagaccaacagctacttcgccaagagcatggagttcctggccaggcccgtgccagctcccaggaccgtgttcaggaacccaccccacccagctcccaggaccaggaccccaagcctggctcccagcagggcctgcagcaggaccagcctggtgagcaccccacccggcgtgaacagggtgatcaccagggaggaactggaggccctgacacccagcaggacccccagcaggtccgtgagcaggactagtctggtgtccaacccacccggcgtgaacagggtgatcaccagggaggaattcgaggccttcgtggcccagcaacagagacggttcgacgccggcgcctacatcttcagcagcgacaccggccagggacacctgcagcaaaagagcgtgaggcagaccgtgctgagcgaggtggtgctggagaggaccgagctggaaatcagctacgcccccaggctggaccaggagaaggaggaactgctcaggaagaaactgcagctgaaccccaccccagccaacaggagcaggtaccagagcaggaaggtggagaacatgaaggccatcaccgccaggcggatcctgcagggcctgggacactacctgaaggccgagggcaaggtggagtgctacaggaccctgcaccccgtgccactgtacagctccagcgtgaacagggccttctccagccccaaggtggccgtggaggcctgcaacgctatgctgaaggagaacttccccaccgtggccagctactgcatcatccccgagtacgacgcctacctggacatggtggacggcgccagctgctgcctggacaccgccagcttctgccccgccaagctgaggagcttccccaagaaacacagctacctggagcccaccatcaggagcgccgtgcccagcgccatccagaacaccctgcagaacgtgctggccgctgccaccaagaggaactgcaacgtgacccagatgagggagctgcccgtgctggacagcgctgccttcaacgtggagtgcttcaagaaatacgcctgcaacaacgagtactgggagaccttcaaggagaaccccatcaggctgaccgaagagaacgtggtgaactacatcaccaagctgaagggccccaaggccgctgccctgttcgctaagacccacaacctgaacatgctgcaggacatcccaatggacaggttcgtgatggacctgaagagggacgtgaaggtgacacccggcaccaagcacaccgaggagaggcccaaggtgcaggtgatccaggccgctgacccactggccaccgcctacctgtgcggcatccacagggagctggtgaggcggctgaacgccgtgctgctgcccaacatccacaccctgttcgacatgagcgccgaggacttcgacgccatcatcgccgagcacttccagcccggcgactgcgtgctggagaccgacatcgccagcttcgacaagagcgaggatgacgctatggccctgaccgctctgatgatcctggaggacctgggcgtggacgccgagctgctcaccctgatcgaggctgccttcggcgagatcagctccatccacctgcccaccaagaccaagttcaagttcggcgctatgatgaaaagcggaatgttcctgaccctgttcgtgaacaccgtgatcaacattgtgatcgccagcagggtgctgcgggagaggctgaccggcagcccctgcgctgccttcatcggcgacgacaacatcgtgaagggcgtgaaaagcgacaagctgatggccgacaggtgcgccacctggctgaacatggaggtgaagatcatcgacgccgtggtgggcgagaaggccccctacttctgcggcggattcatcctgtgcgacagcgtgaccggcaccgcctgcagggtggccgaccccctgaagaggctgttcaagctgggcaagccactggccgctgacgatgagcacgacgatgacaggcggagggccctgcacgaggaaagcaccaggtggaacagggtgggcatcctgagcgagctgtgcaaggccgtggagagcaggtacgagaccgtgggcaccagcatcatcgtgatggctatgaccacactggccagctccgtcaagagcttctcctacctgaggggggcccctataactctctacggctaacctgaatggactacgacatagtctagtccgccaaggccgccaccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgacagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagtccctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaactcgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaatctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 101 - RNA, 9773 nt, synthetic construct
  misc_feature 1..9773: note = Synthetic polynucleotide
  source 1..9773: mol_type = other RNA
attgacggcgtagtacacactattgaatcaaacagccgaccaattgcactaccatcacaatggagaagccagtagtaaacgtagacgtagacccccagagtccgtttgtcgtgcaactgcaaaaaagcttcccgcaatttgaggtagtagcacagcaggtcactccaaatgaccatgctaatgccagagcattttcgcatctggccagtaaactaatcgagctggaggttcctaccacagcgacgatcttggacataggcagcgcaccggctcgtagaatgttttccgagcaccagtatcattgtgtctgccccatgcgtagtccagaagacccggaccgcatgatgaaatatgccagtaaactggcggaaaaagcgtgcaagattacaaacaagaacttgcatgagaagattaaggatctccggaccgtacttgatacgccggatgctgaaacaccatcgctctgctttcacaacgatgttacctgcaacatgcgtgccgaatattccgtcatgcaggacgtgtatatcaacgctcccggaactatctatcatcaggctatgaaaggcgtgcggaccctgtactggattggcttcgacaccacccagttcatgttctcggctatggcaggttcgtaccctgcgtacaacaccaactgggccgacgagaaagtccttgaagcgcgtaacatcggactttgcagcacaaagctgagtgaaggtaggacaggaaaattgtcgataatgaggaagaaggagttgaagcccgggtcgcgggtttatttctccgtaggatcgacactttatccagaacacagagccagcttgcagagctggcatcttccatcggtgttccacttgaatggaaagcagtcgtacacttgccgctgtgatacagtggtgagttgcgaaggctacgtagtgaagaaaatcaccatcagtcccgggatcacgggagaaaccgtgggatacgcggttacacacaatagcgagggcttcttgctatgcaaagttactgacacagtaaaaggagaacgggtatcgttccctgtgtgcacgtacatcccggccaccatatgcgatcagatgactggtataatggccacggatatatcacctgacgatgcacaaaaacttctggttgggctcaaccagcgaattgtcattaacggtaggactaacaggaacaccaacaccatgcaaaattaccttctgccgatcatagcacaagggttcagcaaatgggctaaggagcgcaaggatgatcttgataacgagaaaatgctgggtactagagaacgcaagcttacgtatggctgcttgtgggcgtttcgcactaagaaagtacattcgttttatcgcccacctggaacgcagacctgcgtaaaagtcccagcctcttttagcgcttttcccatgtcgtccgtatggacgacctctttgcccatgtcgctgaggcagaaattgaaactggcattgcaaccaaagaaggaggaaaaactgctgcaggtctcggaggaattagtcatggaggccaaggctgcttttgaggatgctcaggaggaagccagagcggagaagctccgagaagcacttccaccattagtggcagacaaaggcatcgaggcagccgcagaagttgtctgcgaagtggaggggctccaggcggacatcggagcagcattagttgaaaccccgcgcggtcacgtaaggataatacctcaagcaaatgaccgtatgatcggacagtatatcgttgtctcgccaaactctgtgctgaagaatgccaaactcgcaccagcgcacccgctagcagatcaggttaagatcataacacactccggaagatcaggaaggtacgcggtcgaaccatacgacgctaaagtactgatgccagcaggaggtgccgtaccatggccagaattcctagcactgagtgagagcgccacgttagtgtacaacgaaagagagtttgtgaaccgcaaactataccacattgccatgcatggccccgccaagaatacagaagaggagcagtacaaggttacaaaggcagagcttgcagaaacagagtacgtgtttgacgtggacaagaagcgttgcgttaagaaggaagaagcctcaggtctggtcctctcgggagaactgaccaaccctccctatcatgagctagctctggagggactgaagacccgacctgcggtcccgtacaaggtcgaaacaataggagtgataggcacaccggggtcgggcaagtcagctattatcaagtcaactgtcacggcacgagatcttgttaccagcggaaagaaagaaaattgtcgcgaaattgaggccgacgtgctaagactgaggggtatgcagattacgtcgaagacagtagattcggttatgctcaacggatgccacaaagccgtagaagtgctgtacgttgacgaagcgttcgcgtgccacgcaggagcactacttgccttgattgctatcgtcaggccccgcaagaaggtagtactatgcggagaccccatgcaatgcggattcttcaacatgatgcaactaaaggtacatttcaatcaccctgaaaaagacatatgcaccaagacattctacaagtatatctcccggcgttgcacacagccagttacagctattgtatcgacactgcattacgatggaaagatgaaaaccacgaacccgtgcaagaagaacattgaaatcgatattacaggggccacaaagccgaagccaggggatatcatcctgacatgtttccgcgggtgggttaagcaattgcaaatcgactatcccggacatgaagtaatgacagccgcggcctcacaagggctaaccagaaaaggagtgtatgccgtccggcaaaaagtcaatgaaaacccactgtacgcgatcacatcagagcatgtgaacgtgttgctcacccgcactgaggacaggctagtgtggaaaaccttgcagggcgacccatggattaagcagctcactaacatacctaaaggaaactttcaggctactatagaggactgggaagctgaacacaagggaataattgctgcaataaacagccccactccccgtgccaatccgttcagctgcaagaccaacgtttgctgggcgaaagcattggaaccgatactagccacggccggtatcgtacttaccggttgccagtggagcgaactgttcccacagtttgcggatgacaaaccacattcggccatttacgccttagacgtaatttgcattaagtttttcggcatggacttgacaagcggactgttttctaaacagagcatcccactaacgtaccatcccgccgattcagcgaggccggtagctcattgggacaacagcccaggaacccgcaagtatgggtacgatcacgccattgccgccgaactctcccgtagatttccggtgttccagctagctgggaagggcacacaacttgatttgcagacggggagaaccagagttatctctgcacagcataacctggtcccggtgaaccgcaatcttcctcacgccttagtccccgagtacaaggagaagcaacccggcccggtcgaaaaattcttgaaccagttcaaacaccactcagtacttgtggtatcagaggaaaaaattgaagctccccgtaagagaatcgaatggatcgccccgattggcatagccggtgcagataagaactacaacctggctttcgggtttccgccgcaggcacggtacgacctggtgttcatcaacattggaactaaatacagaaaccaccactttcagcagtgcgaagaccatgcggcgaccttaaaaaccctttcgcgttcggccctgaattgccttaacccaggaggcaccctcgtggtgaagtcctatggctacgccgaccgcaacagtgaggacgtagtcaccgctcttgccagaaagtttgtcagggtgtctgcagcgagaccagattgtgtctcaagcaatacagaaatgtacctgattttccgacaactagacaacagccgtacacggcaattcaccccgcaccatctgaattgcgtgatttcgtccgtgtatgagggtacaagagatggagttggagccgcgccgtcataccgcaccaaaagggagaatattgctgactgtcaagaggaagcagttgtcaacgcagccaatccgctgggtagaccaggcgaaggagtctgccgtgccatctataaacgttggccgaccagttttaccgattcagccacggagacaggcaccgcaagaatgactgtgtgcctaggaaagaaagtgatccacgcggtcggccctgatttccggaagcacccagaagcagaagccttgaaattgctacaaaacgcctaccatgcagtggcagacttagtaaatgaacataacatcaagtctgtcgccattccactgctatctacaggcatttacgcagccggaaaagaccgccttgaagtatcacttaactgcttgacaaccgcgctagacagaactgacgcggacgtaaccatctattgcctggataagaagtggaaggaaagaatcgacgcggcactccaacttaaggagtctgtaacagagctgaaggatgaagatatggagatcgacgatgagttagtatggatccatccagacagttgcttgaagggaagaaagggattcagtactacaaaaggaaaattgtattcgtacttcgaaggcaccaaattccatcaagcagcaaaagacatggcggagataaaggtcctgttccctaatgaccaggaaagtaatgaacaactgtgtgcctacatattgggtgagaccatggaagcaatccgcgaaaagtgcccggtcgaccataacccgtcgtctagcccgcccaaaacgttgccgtgcctttgcatgtatgccatgacgccagaaagggtccacagacttagaagcaataacgtcaaagaagttacagtatgctcctccaccccccttcctaagcacaaaattaagaatgttcagaaggttcagtgcacgaaagtagtcctgtttaatccgcacactcccgcattcgttcccgcccgtaagtacatagaagtgccagaacagcctaccgctcctcctgcacaggccgaggaggcccccgaagttgtagcgacaccgtcaccatctacagctgataacacctcgcttgatgtcacagacatctcactggatatggatgacagtagcgaaggctcacttttttcgagctttagcggatcggacaactctattactagtatggacagttggtcgtcaggacctagttcactagagatagtagaccgaaggcaggtggtggtggctgacgttcatgccgtccaagagcctgcccctattccaccgccaaggctaaagaagatggcccgcctggcagcggcaagaaaagagcccactccaccggcaagcaatagctctgagtccctccacctctcttttggtggggtatccatgtccctcggatcaattttcgacggagagacggcccgccaggcagcggtacaacccctggcaacaggccccacggatgtgcctatgtctttcggatcgttttccgacggagagattgatgagctgagccgcagagtaactgagtccgaacccgtcctgtttggatcatttgaaccgggcgaagtgaactcaattatatcgtcccgatcagccgtatcttttcctctacgcaagcagagacgtagacgcaggagcaggaggactgaatactgactaaccggggtaggtgggtacatattttcgacggacacaggccctgggcacttgcaaaagaagtccgttctgcagaaccagcttacagaaccgaccttggagcgcaatgtcctggaaagaattcatgccccggtgctcgacacgtcgaaagaggaacaactcaaactcaggtaccagatgatgcccaccgaagccaacaaaagtaggtaccagtctcgtaaagtagaaaatcagaaagccataaccactgagcgactactgtcaggactacgactgtataactctgccacagatcagccagaatgctataagatcacctatccgaaaccattgtactccagtagcgtaccggcgaactactccgatccacagttcgctgtagctgtctgtaacaactatctgcatgagaactatccgacagtagcatcttatcagattactgacgagtacgatgcttacttggatatggtagacgggacagtcgcctgcctggacactgcaaccttctgccccgctaagcttagaagttacccgaaaaaacatgagtatagagccccgaatatccgcagtgcggttccatcagcgatgcagaacacgctacaaaatgtgctcattgccgcaactaaaagaaattgcaacgtcacgcagatgcgtgaactgccaacactggactcagcgacattcaatgtcgaatgctttcgaaaatatgcatgtaatgacgagtattgggaggagttcgctcggaagccaattaggattaccactgagtttgtcaccgcatatgtagctagactgaaaggccctaaggccgccgcactatttgcaaagacgtataatttggtcccattgcaagaagtgcctatggatagattcgtcatggacatgaaaagagacgtgaaagttacaccaggcacgaaacacacagaagaaagaccgaaagtacaagtgatacaagccgcagaacccctggcgactgcttacttatgcgggattcaccgggaattagtgcgtaggcttacggccgtcttgcttccaaacattcacacgctttttgacatgtcggcggaggattttgatgcaatcatagcagaacacttcaagcaaggcgacccggtactggagacggatatcgcatcattcgacaaaagccaagacgacgctatggcgttaaccggtctgatgatcttggaggacctgggtgtggatcaaccactactcgacttgatcgagtgcgcctttggagaaatatcatccacccatctacctacgggtactcgttttaaattcggggcgatgatgaaatccggaatgttcctcacactttttgtcaacacagttttgaatgtcgttatcgccagcagagtactagaggagcggcttaaaacgtccagatgtgcagcgttcattggcgacgacaacatcatacatggagtagtatctgacaaagaaatggctgagaggtgcgccacctggctcaacatggaggttaagatcatcgacgcagtcatcggtgagagaccaccttacttctgcggcggatttatcttgcaagattcggttacttccacagcgtgccgcgtggcggatcccctgaaaaggctgtttaagttgggtaaaccgctcccagccgacgacgagcaagacgaagacagaagacgcgctctgctagatgaaacaaaggcgtggtttagagtaggtataacaggcactttagcagtggccgtgacgacccggtatgaggtagacaatattacacctgtcctactggcattgagaacttttgcccagagcaaaagagcattccaagccatcagaggggaaataaagcatctctacggtggtcctaaatagtcagcatagtacatttcatctgactaatactacaacaccaccaccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgacagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagtccctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaaacgcgtgctagaccatggatcctagacgctacgccccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgcataacttttattatttcttttattaatcaacaaaattttgtttttaacatttcaaaaaaaaaaaaaaaaaaaaaaaaatctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 102 - RNA, 2086 nt, synthetic construct
  misc_feature 1..2086: note = Synthetic polynucleotide
  source 1..2086: mol_type = other RNA
aggaaacttaagtcaacacaacatatacaaaacaaacgaatctcaagcaatcaagcattctacttctattgcagcaatttaaatcatttcttttaaagcaaaagcaattttctgaaaattttcaccatttacgaacgatagccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgacagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagtccctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 103 - RNA, 8095 nt, synthetic construct
  misc_feature 1..8095: note = Synthetic polynucleotide
  source 1..8095: mol_type = other RNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgccgtcgacggccccaccagcctgtaccaccaggccaacaagggcgtgagggtggcctactggatcggcttcgacaccacacccttcatgttcaagaacctggccggcgcctaccccagctacagcaccaactgggccgacgagaccgtgctgaccgccaggaacatcggcctgtgcagcagcgacgtgatggagaggagccggagaggcatgagcatcctgaggaagaaatacctgaagcccagcaacaacgtgctgttcagcgtgggcagcaccatctaccacgagaagagggacctgctcaggagctggcacctgcccagcgtgttccacctgaggggcaagcagaactacacctgcaggtgcgagaccatcgtgagctgcgacggctacgtggtgaagaggatcgccatcagccccggcctgtacggcaagcccagcggctacgccgctacaatgcacagggagggcttcctgtgctgcaaggtgaccgacaccctgaacggcgagagggtgagcttccccgtgtgcacctacgtgcccgccaccctgtgcgaccagatgaccggcatcctggccaccgacgtgagcgccgacgacgcccagaagctgctcgtgggcctgaaccagaggatcgtggtcaacggcaggacccagaggaacaccaacacaatgaagaactacctgctgcccgtggtggcccaggctttcgccaggtgggccaaggagtacaaggaggaccaggaagacgagaggcccctgggcctgagggacaggcagctggtgatgggctgctgctgggccttcaggcggcacaagatcaccagcatctacaagaggcccgacacccagaccatcatcaaggtgaacagcgacttccacagcttcgtgctgcccaggatcggcagcaacaccctggagatcggcctgaggacccggatcaggaagatgctggaggaacacaaggagcccagcccactgatcaccgccgaggacgtgcaggaggccaagtgcgctgccgacgaggccaaggaggtgagggaggccgaggaactgagggccgccctgccacccctggctgccgacgtggaggaacccaccctggaagccgacgtggacctgatgctgcaggaggccggcgccggaagcgtggagacacccaggggcctgatcaaggtgaccagctacgacggcgaggacaagatcggcagctacgccgtgctgagcccacaggccgtgctgaagtccgagaagctgagctgcatccacccactggccgagcaggtgatcgtgatcacccacagcggcaggaagggcaggtacgccgtggagccctaccacggcaaggtggtcgtgcccgagggccacgccatccccgtgcaggacttccaggccctgagcgagagcgccaccatcgtgtacaacgagagggagttcgtgaacaggtacctgcaccatatcgccacccacggcggagccctgaacaccgacgaggaatactacaagaccgtgaagcccagcgagcacgacggcgagtacctgtacgacatcgacaggaagcagtgcgtgaagaaagagctggtgaccggcctgggactgaccggcgagctggtggacccacccttccacgagttcgcctacgagagcctgaggaccagacccgccgctccctaccaggtgcccaccatcggcgtgtacggcgtgcccggcagcggaaagagcggcatcatcaagagcgccgtgaccaagaaagacctggtggtcagcgccaagaaagagaactgcgccgagatcatcagggacgtgaagaagatgaaaggcctggacgtgaacgcgcgcaccgtggacagcgtgctgctgaacggctgcaagcaccccgtggagaccctgtacatcgacgaggccttcgcttgccacgccggcaccctgagggccctgatcgccatcatcaggcccaagaaagccgtgctgtgcggcgaccccaagcagtgcggcttcttcaacatgatgtgcctgaaggtgcacttcaaccacgagatctgcacccaggtgttccacaagagcatcagcaggcggtgcaccaagagcgtgaccagcgtcgtgagcaccctgttctacgacaagaaaatgaggaccaccaaccccaaggagaccaaaatcgtgatcgacaccacaggcagcaccaagcccaagcaggacgacctgatcctgacctgcttcaggggctgggtgaagcagctgcagatcgactacaagggcaacgagatcatgaccgccgctgccagccagggcctgaccaggaagggcgtgtacgccgtgaggtacaaggtgaacgagaacccactgtacgctcccaccagcgagcacgtgaacgtgctgctgaccaggaccgaggacaggatcgtgtggaagaccctggccggcgacccctggatcaagaccctgaccgccaagtaccccggcaacttcaccgccaccatcgaagagtggcaggccgagcacgacgccatcatgaggcacatcctggagaggcccgaccccaccgacgtgttccagaacaaggccaacgtgtgctgggccaaggccctggtgcccgtgctgaagaccgccggcatcgacatgaccacagagcagtggaacaccgtggactacttcgagaccgacaaggcccacagcgccgagatcgtgctgaaccagctgtgcgtgaggttcttcggcctggacctggacagcggcctgttcagcgcccccaccgtgccactgagcatcaggaacaaccactgggacaacagccccagcccaaacatgtacggcctgaacaaggaggtggtcaggcagctgagcaggcggtacccacagctgcccagggccgtggccaccggcagggtgtacgacatgaacaccggcaccctgaggaactacgaccccaggatcaacctggtgcccgtgaacaggcggctgccccacgccctggtgctgcaccacaacgagcacccacagagcgacttcagctccttcgtgagcaagctgaaaggcaggaccgtgctggtcgtgggcgagaagctgagcgtgcccggcaagatggtggactggctgagcgacaggcccgaggccaccttccgggccaggctggacctcggcatccccggcgacgtgcccaagtacgacatcatcttcgtgaacgtcaggaccccatacaagtaccaccattaccagcagtgcgaggaccacgccatcaagctgagcatgctgaccaagaaggcctgcctgcacctgaaccccggaggcacctgcgtgagcatcggctacggctacgccgacagggccagcgagagcatcattggcgccatcgccaggctgttcaagttcagcagggtgtgcaaacccaagagcagcctggaggaaaccgaggtgctgttcgtgttcatcggctacgaccggaaggccaggacccacaacccctacaagctgagcagcaccctgacaaacatctacaccggcagcaggctgcacgaggccggctgcgcccccagctaccacgtggtcaggggcgatatcgccaccgccaccgagggcgtgatcatcaacgctgccaacagcaagggccagcccggaggcggagtgtgcggcgccctgtacaagaagttccccgagagcttcgacctgcagcccatcgaggtgggcaaggccaggctggtgaagggcgccgctaagcacatcatccacgccgtgggccccaacttcaacaaggtgagcgaggtggaaggcgacaagcagctggccgaagcctacgagagcatcgccaagatcgtgaacgacaataactacaagagcgtggccatcccactgctcagcaccggcatcttcagcggcaacaaggacaggctgacccagagcctgaaccacctgctcaccgccctggacaccaccgatgccgacgtggccatctactgcagggacaagaagtgggagatgaccctgaaggaggccgtggccaggcgggaggccgtggaagagatctgcatcagcgacgactccagcgtgaccgagcccgacgccgagctggtgagggtgcaccccaagagctccctggccggcaggaagggctacagcaccagcgacggcaagaccttcagctacctggagggcaccaagttccaccaggccgctaaggacatcgccgagatcaacgctatgtggcccgtggccaccgaggccaacgagcaggtgtgcatgtacatcctgggcgagagcatgtccagcatcaggagcaagtgccccgtggaggaaagcgaggccagcacaccacccagcaccctgccctgcctgtgcatccacgctatgacacccgagagggtgcagcggctgaaggccagcaggcccgagcagatcaccgtgtgcagctccttcccactgcccaagtacaggatcaccggcgtgcagaagatccagtgcagccagcccatcctgttcagcccaaaggtgcccgcctacatccaccccaggaagtacctggtggagaccccacccgtggacgagacacccgagccaagcgccgagaaccagagcaccgagggcacacccgagcagccacccctgatcaccgaggacgagacaaggacccggaccccagagcccatcattatcgaggaagaggaagaggacagcatcagcctgctgagcgacggccccacccaccaggtgctgcaggtggaggccgacatccacggcccacccagcgtgtccagctccagctggagcatcccacacgccagcgacttcgacgtggacagcctgagcatcctggacaccctggagggcgccagcgtgacctccggcgccaccagcgccgagaccaacagctacttcgccaagagcatggagttcctggccaggcccgtgccagctcccaggaccgtgttcaggaacccaccccacccagctcccaggaccaggaccccaagcctggctcccagcagggcctgcagcaggaccagcctggtgagcaccccacccggcgtgaacagggtgatcaccagggaggaactggaggccctgacacccagcaggacccccagcaggtccgtgagcaggactagtctggtgtccaacccacccggcgtgaacagggtgatcaccagggaggaattcgaggccttcgtggcccagcaacagagacggttcgacgccggcgcctacatcttcagcagcgacaccggccagggacacctgcagcaaaagagcgtgaggcagaccgtgctgagcgaggtggtgctggagaggaccgagctggaaatcagctacgcccccaggctggaccaggagaaggaggaactgctcaggaagaaactgcagctgaaccccaccccagccaacaggagcaggtaccagagcaggaaggtggagaacatgaaggccatcaccgccaggcggatcctgcagggcctgggacactacctgaaggccgagggcaaggtggagtgctacaggaccctgcaccccgtgccactgtacagctccagcgtgaacagggccttctccagccccaaggtggccgtggaggcctgcaacgctatgctgaaggagaacttccccaccgtggccagctactgcatcatccccgagtacgacgcctacctggacatggtggacggcgccagctgctgcctggacaccgccagcttctgccccgccaagctgaggagcttccccaagaaacacagctacctggagcccaccatcaggagcgccgtgcccagcgccatccagaacaccctgcagaacgtgctggccgctgccaccaagaggaactgcaacgtgacccagatgagggagctgcccgtgctggacagcgctgccttcaacgtggagtgcttcaagaaatacgcctgcaacaacgagtactgggagaccttcaaggagaaccccatcaggctgaccgaagagaacgtggtgaactacatcaccaagctgaagggccccaaggccgctgccctgttcgctaagacccacaacctgaacatgctgcaggacatcccaatggacaggttcgtgatggacctgaagagggacgtgaaggtgacacccggcaccaagcacaccgaggagaggcccaaggtgcaggtgatccaggccgctgacccactggccaccgcctacctgtgcggcatccacagggagctggtgaggcggctgaacgccgtgctgctgcccaacatccacaccctgttcgacatgagcgccgaggacttcgacgccatcatcgccgagcacttccagcccggcgactgcgtgctggagaccgacatcgccagcttcgacaagagcgaggatgacgctatggccctgaccgctctgatgatcctggaggacctgggcgtggacgccgagctgctcaccctgatcgaggctgccttcggcgagatcagctccatccacctgcccaccaagaccaagttcaagttcggcgctatgatgaaaagcggaatgttcctgaccctgttcgtgaacaccgtgatcaacattgtgatcgccagcagggtgctgcgggagaggctgaccggcagcccctgcgctgccttcatcggcgacgacaacatcgtgaagggcgtgaaaagcgacaagctgatggccgacaggtgcgccacctggctgaacatggaggtgaagatcatcgacgccgtggtgggcgagaaggccccctacttctgcggcggattcatcctgtgcgacagcgtgaccggcaccgcctgcagggtggccgaccccctgaagaggctgttcaagctgggcaagccactggccgctgacgatgagcacgacgatgacaggcggagggccctgcacgaggaaagcaccaggtggaacagggtgggcatcctgagcgagctgtgcaaggccgtggagagcaggtacgagaccgtgggcaccagcatcatcgtgatggctatgaccacactggccagctccgtcaagagcttctcctacctgaggggggcccctataactctctacggctaacctgaatggactacgacatagtctagtccgccaaggccgccacccatgaagttggtggttgtgggggccgggggtgttggcaaaagcgcccttacaatttgactcgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaatctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 104 - RNA, 8120 nt, synthetic construct
  misc_feature 1..8120: note = Synthetic polynucleotide
  source 1..8120: mol_type = other RNA
attgacggcgtagtacacactattgaatcaaacagccgaccaattgcactaccatcacaatggagaagccagtagtaaacgtagacgtagacccccagagtccgtttgtcgtgcaactgcaaaaaagcttcccgcaatttgaggtagtagcacagcaggtcactccaaatgaccatgctaatgccagagcattttcgcatctggccagtaaactaatcgagctggaggttcctaccacagcgacgatcttggacataggcagcgcaccggctcgtagaatgttttccgagcaccagtatcattgtgtctgccccatgcgtagtccagaagacccggaccgcatgatgaaatatgccagtaaactggcggaaaaagcgtgcaagattacaaacaagaacttgcatgagaagattaaggatctccggaccgtacttgatacgccggatgctgaaacaccatcgctctgctttcacaacgatgttacctgcaacatgcgtgccgaatattccgtcatgcaggacgtgtatatcaacgctcccggaactatctatcatcaggctatgaaaggcgtgcggaccctgtactggattggcttcgacaccacccagttcatgttctcggctatggcaggttcgtaccctgcgtacaacaccaactgggccgacgagaaagtccttgaagcgcgtaacatcggactttgcagcacaaagctgagtgaaggtaggacaggaaaattgtcgataatgaggaagaaggagttgaagcccgggtcgcgggtttatttctccgtaggatcgacactttatccagaacacagagccagcttgcagagctggcatcttccatcggtgttccacttgaatggaaagcagtcgtacacttgccgctgtgatacagtggtgagttgcgaaggctacgtagtgaagaaaatcaccatcagtcccgggatcacgggagaaaccgtgggatacgcggttacacacaatagcgagggcttcttgctatgcaaagttactgacacagtaaaaggagaacgggtatcgttccctgtgtgcacgtacatcccggccaccatatgcgatcagatgactggtataatggccacggatatatcacctgacgatgcacaaaaacttctggttgggctcaaccagcgaattgtcattaacggtaggactaacaggaacaccaacaccatgcaaaattaccttctgccgatcatagcacaagggttcagcaaatgggctaaggagcgcaaggatgatcttgataacgagaaaatgctgggtactagagaacgcaagcttacgtatggctgcttgtgggcgtttcgcactaagaaagtacattcgttttatcgcccacctggaacgcagacctgcgtaaaagtcccagcctcttttagcgcttttcccatgtcgtccgtatggacgacctctttgcccatgtcgctgaggcagaaattgaaactggcattgcaaccaaagaaggaggaaaaactgctgcaggtctcggaggaattagtcatggaggccaaggctgcttttgaggatgctcaggaggaagccagagcggagaagctccgagaagcacttccaccattagtggcagacaaaggcatcgaggcagccgcagaagttgtctgcgaagtggaggggctccaggcggacatcggagcagcattagttgaaaccccgcgcggtcacgtaaggataatacctcaagcaaatgaccgtatgatcggacagtatatcgttgtctcgccaaactctgtgctgaagaatgccaaactcgcaccagcgcacccgctagcagatcaggttaagatcataacacactccggaagatcaggaaggtacgcggtcgaaccatacgacgctaaagtactgatgccagcaggaggtgccgtaccatggccagaattcctagcactgagtgagagcgccacgttagtgtacaacgaaagagagtttgtgaaccgcaaactataccacattgccatgcatggccccgccaagaatacagaagaggagcagtacaaggttacaaaggcagagcttgcagaaacagagtacgtgtttgacgtggacaagaagcgttgcgttaagaaggaagaagcctcaggtctggtcctctcgggagaactgaccaaccctccctatcatgagctagctctggagggactgaagacccgacctgcggtcccgtacaaggtcgaaacaataggagtgataggcacaccggggtcgggcaagtcagctattatcaagtcaactgtcacggcacgagatcttgttaccagcggaaagaaagaaaattgtcgcgaaattgaggccgacgtgctaagactgaggggtatgcagattacgtcgaagacagtagattcggttatgctcaacggatgccacaaagccgtagaagtgctgtacgttgacgaagcgttcgcgtgccacgcaggagcactacttgccttgattgctatcgtcaggccccgcaagaaggtagtactatgcggagaccccatgcaatgcggattcttcaacatgatgcaactaaaggtacatttcaatcaccctgaaaaagacatatgcaccaagacattctacaagtatatctcccggcgttgcacacagccagttacagctattgtatcgacactgcattacgatggaaagatgaaaaccacgaacccgtgcaagaagaacattgaaatcgatattacaggggccacaaagccgaagccaggggatatcatcctgacatgtttccgcgggtgggttaagcaattgcaaatcgactatcccggacatgaagtaatgacagccgcggcctcacaagggctaaccagaaaaggagtgtatgccgtccggcaaaaagtcaatgaaaacccactgtacgcgatcacatcagagcatgtgaacgtgttgctcacccgcactgaggacaggctagtgtggaaaaccttgcagggcgacccatggattaagcagctcactaacatacctaaaggaaactttcaggctactatagaggactgggaagctgaacacaagggaataattgctgcaataaacagccccactccccgtgccaatccgttcagctgcaagaccaacgtttgctgggcgaaagcattggaaccgatactagccacggccggtatcgtacttaccggttgccagtggagcgaactgttcccacagtttgcggatgacaaaccacattcggccatttacgccttagacgtaatttgcattaagtttttcggcatggacttgacaagcggactgttttctaaacagagcatcccactaacgtaccatcccgccgattcagcgaggccggtagctcattgggacaacagcccaggaacccgcaagtatgggtacgatcacgccattgccgccgaactctcccgtagatttccggtgttccagctagctgggaagggcacacaacttgatttgcagacggggagaaccagagttatctctgcacagcataacctggtcccggtgaaccgcaatcttcctcacgccttagtccccgagtacaaggagaagcaacccggcccggtcgaaaaattcttgaaccagttcaaacaccactcagtacttgtggtatcagaggaaaaaattgaagctccccgtaagagaatcgaatggatcgccccgattggcatagccggtgcagataagaactacaacctggctttcgggtttccgccgcaggcacggtacgacctggtgttcatcaacattggaactaaatacagaaaccaccactttcagcagtgcgaagaccatgcggcgaccttaaaaaccctttcgcgttcggccctgaattgccttaacccaggaggcaccctcgtggtgaagtcctatggctacgccgaccgcaacagtgaggacgtagtcaccgctcttgccagaaagtttgtcagggtgtctgcagcgagaccagattgtgtctcaagcaatacagaaatgtacctgattttccgacaactagacaacagccgtacacggcaattcaccccgcaccatctgaattgcgtgatttcgtccgtgtatgagggtacaagagatggagttggagccgcgccgtcataccgcaccaaaagggagaatattgctgactgtcaagaggaagcagttgtcaacgcagccaatccgctgggtagaccaggcgaaggagtctgccgtgccatctataaacgttggccgaccagttttaccgattcagccacggagacaggcaccgcaagaatgactgtgtgcctaggaaagaaagtgatccacgcggtcggccctgatttccggaagcacccagaagcagaagccttgaaattgctacaaaacgcctaccatgcagtggcagacttagtaaatgaacataacatcaagtctgtcgccattccactgctatctacaggcatttacgcagccggaaaagaccgccttgaagtatcacttaactgcttgacaaccgcgctagacagaactgacgcggacgtaaccatctattgcctggataagaagtggaaggaaagaatcgacgcggcactccaacttaaggagtctgtaacagagctgaaggatgaagatatggagatcgacgatgagttagtatggatccatccagacagttgcttgaagggaagaaagggattcagtactacaaaaggaaaattgtattcgtacttcgaaggcaccaaattccatcaagcagcaaaagacatggcggagataaaggtcctgttccctaatgaccaggaaagtaatgaacaactgtgtgcctacatattgggtgagaccatggaagcaatccgcgaaaagtgcccggtcgaccataacccgtcgtctagcccgcccaaaacgttgccgtgcctttgcatgtatgccatgacgccagaaagggtccacagacttagaagcaataacgtcaaagaagttacagtatgctcctccaccccccttcctaagcacaaaattaagaatgttcagaaggttcagtgcacgaaagtagtcctgtttaatccgcacactcccgcattcgttcccgcccgtaagtacatagaagtgccagaacagcctaccgctcctcctgcacaggccgaggaggcccccgaagttgtagcgacaccgtcaccatctacagctgataacacctcgcttgatgtcacagacatctcactggatatggatgacagtagcgaaggctcacttttttcgagctttagcggatcggacaactctattactagtatggacagttggtcgtcaggacctagttcactagagatagtagaccgaaggcaggtggtggtggctgacgttcatgccgtccaagagcctgcccctattccaccgccaaggctaaagaagatggcccgcctggcagcggcaagaaaagagcccactccaccggcaagcaatagctctgagtccctccacctctcttttggtggggtatccatgtccctcggatcaattttcgacggagagacggcccgccaggcagcggtacaacccctggcaacaggccccacggatgtgcctatgtctttcggatcgttttccgacggagagattgatgagctgagccgcagagtaactgagtccgaacccgtcctgtttggatcatttgaaccgggcgaagtgaactcaattatatcgtcccgatcagccgtatcttttcctctacgcaagcagagacgtagacgcaggagcaggaggactgaatactgactaaccggggtaggtgggtacatattttcgacggacacaggccctgggcacttgcaaaagaagtccgttctgcagaaccagcttacagaaccgaccttggagcgcaatgtcctggaaagaattcatgccccggtgctcgacacgtcgaaagaggaacaactcaaactcaggtaccagatgatgcccaccgaagccaacaaaagtaggtaccagtctcgtaaagtagaaaatcagaaagccataaccactgagcgactactgtcaggactacgactgtataactctgccacagatcagccagaatgctataagatcacctatccgaaaccattgtactccagtagcgtaccggcgaactactccgatccacagttcgctgtagctgtctgtaacaactatctgcatgagaactatccgacagtagcatcttatcagattactgacgagtacgatgcttacttggatatggtagacgggacagtcgcctgcctggacactgcaaccttctgccccgctaagcttagaagttacccgaaaaaacatgagtatagagccccgaatatccgcagtgcggttccatcagcgatgcagaacacgctacaaaatgtgctcattgccgcaactaaaagaaattgcaacgtcacgcagatgcgtgaactgccaacactggactcagcgacattcaatgtcgaatgctttcgaaaatatgcatgtaatgacgagtattgggaggagttcgctcggaagccaattaggattaccactgagtttgtcaccgcatatgtagctagactgaaaggccctaaggccgccgcactatttgcaaagacgtataatttggtcccattgcaagaagtgcctatggatagattcgtcatggacatgaaaagagacgtgaaagttacaccaggcacgaaacacacagaagaaagaccgaaagtacaagtgatacaagccgcagaacccctggcgactgcttacttatgcgggattcaccgggaattagtgcgtaggcttacggccgtcttgcttccaaacattcacacgctttttgacatgtcggcggaggattttgatgcaatcatagcagaacacttcaagcaaggcgacccggtactggagacggatatcgcatcattcgacaaaagccaagacgacgctatggcgttaaccggtctgatgatcttggaggacctgggtgtggatcaaccactactcgacttgatcgagtgcgcctttggagaaatatcatccacccatctacctacgggtactcgttttaaattcggggcgatgatgaaatccggaatgttcctcacactttttgtcaacacagttttgaatgtcgttatcgccagcagagtactagaggagcggcttaaaacgtccagatgtgcagcgttcattggcgacgacaacatcatacatggagtagtatctgacaaagaaatggctgagaggtgcgccacctggctcaacatggaggttaagatcatcgacgcagtcatcggtgagagaccaccttacttctgcggcggatttatcttgcaagattcggttacttccacagcgtgccgcgtggcggatcccctgaaaaggctgtttaagttgggtaaaccgctcccagccgacgacgagcaagacgaagacagaagacgcgctctgctagatgaaacaaaggcgtggtttagagtaggtataacaggcactttagcagtggccgtgacgacccggtatgaggtagacaatattacacctgtcctactggcattgagaacttttgcccagagcaaaagagcattccaagccatcagaggggaaataaagcatctctacggtggtcctaaatagtcagcatagtacatttcatctgactaatactacaacaccaccaccacgcgtgctagaccatggatcctagacgctacgccccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgcataacttttattatttcttttattaatcaacaaaattttgtttttaacatttcaaaaaaaaaaaaaaaaaaaaaaaaatctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 105 - RNA, 8875 nt, synthetic construct
  misc_feature 1..8875: note = Synthetic polynucleotide
  source 1..8875: mol_type = other RNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgccgtcgacggccccaccagcctgtaccaccaggccaacaagggcgtgagggtggcctactggatcggcttcgacaccacacccttcatgttcaagaacctggccggcgcctaccccagctacagcaccaactgggccgacgagaccgtgctgaccgccaggaacatcggcctgtgcagcagcgacgtgatggagaggagccggagaggcatgagcatcctgaggaagaaatacctgaagcccagcaacaacgtgctgttcagcgtgggcagcaccatctaccacgagaagagggacctgctcaggagctggcacctgcccagcgtgttccacctgaggggcaagcagaactacacctgcaggtgcgagaccatcgtgagctgcgacggctacgtggtgaagaggatcgccatcagccccggcctgtacggcaagcccagcggctacgccgctacaatgcacagggagggcttcctgtgctgcaaggtgaccgacaccctgaacggcgagagggtgagcttccccgtgtgcacctacgtgcccgccaccctgtgcgaccagatgaccggcatcctggccaccgacgtgagcgccgacgacgcccagaagctgctcgtgggcctgaaccagaggatcgtggtcaacggcaggacccagaggaacaccaacacaatgaagaactacctgctgcccgtggtggcccaggctttcgccaggtgggccaaggagtacaaggaggaccaggaagacgagaggcccctgggcctgagggacaggcagctggtgatgggctgctgctgggccttcaggcggcacaagatcaccagcatctacaagaggcccgacacccagaccatcatcaaggtgaacagcgacttccacagcttcgtgctgcccaggatcggcagcaacaccctggagatcggcctgaggacccggatcaggaagatgctggaggaacacaaggagcccagcccactgatcaccgccgaggacgtgcaggaggccaagtgcgctgccgacgaggccaaggaggtgagggaggccgaggaactgagggccgccctgccacccctggctgccgacgtggaggaacccaccctggaagccgacgtggacctgatgctgcaggaggccggcgccggaagcgtggagacacccaggggcctgatcaaggtgaccagctacgacggcgaggacaagatcggcagctacgccgtgctgagcccacaggccgtgctgaagtccgagaagctgagctgcatccacccactggccgagcaggtgatcgtgatcacccacagcggcaggaagggcaggtacgccgtggagccctaccacggcaaggtggtcgtgcccgagggccacgccatccccgtgcaggacttccaggccctgagcgagagcgccaccatcgtgtacaacgagagggagttcgtgaacaggtacctgcaccatatcgccacccacggcggagccctgaacaccgacgaggaatactacaagaccgtgaagcccagcgagcacgacggcgagtacctgtacgacatcgacaggaagcagtgcgtgaagaaagagctggtgaccggcctgggactgaccggcgagctggtggacccacccttccacgagttcgcctacgagagcctgaggaccagacccgccgctccctaccaggtgcccaccatcggcgtgtacggcgtgcccggcagcggaaagagcggcatcatcaagagcgccgtgaccaagaaagacctggtggtcagcgccaagaaagagaactgcgccgagatcatcagggacgtgaagaagatgaaaggcctggacgtgaacgcgcgcaccgtggacagcgtgctgctgaacggctgcaagcaccccgtggagaccctgtacatcgacgaggccttcgcttgccacgccggcaccctgagggccctgatcgccatcatcaggcccaagaaagccgtgctgtgcggcgaccccaagcagtgcggcttcttcaacatgatgtgcctgaaggtgcacttcaaccacgagatctgcacccaggtgttccacaagagcatcagcaggcggtgcaccaagagcgtgaccagcgtcgtgagcaccctgttctacgacaagaaaatgaggaccaccaaccccaaggagaccaaaatcgtgatcgacaccacaggcagcaccaagcccaagcaggacgacctgatcctgacctgcttcaggggctgggtgaagcagctgcagatcgactacaagggcaacgagatcatgaccgccgctgccagccagggcctgaccaggaagggcgtgtacgccgtgaggtacaaggtgaacgagaacccactgtacgctcccaccagcgagcacgtgaacgtgctgctgaccaggaccgaggacaggatcgtgtggaagaccctggccggcgacccctggatcaagaccctgaccgccaagtaccccggcaacttcaccgccaccatcgaagagtggcaggccgagcacgacgccatcatgaggcacatcctggagaggcccgaccccaccgacgtgttccagaacaaggccaacgtgtgctgggccaaggccctggtgcccgtgctgaagaccgccggcatcgacatgaccacagagcagtggaacaccgtggactacttcgagaccgacaaggcccacagcgccgagatcgtgctgaaccagctgtgcgtgaggttcttcggcctggacctggacagcggcctgttcagcgcccccaccgtgccactgagcatcaggaacaaccactgggacaacagccccagcccaaacatgtacggcctgaacaaggaggtggtcaggcagctgagcaggcggtacccacagctgcccagggccgtggccaccggcagggtgtacgacatgaacaccggcaccctgaggaactacgaccccaggatcaacctggtgcccgtgaacaggcggctgccccacgccctggtgctgcaccacaacgagcacccacagagcgacttcagctccttcgtgagcaagctgaaaggcaggaccgtgctggtcgtgggcgagaagctgagcgtgcccggcaagatggtggactggctgagcgacaggcccgaggccaccttccgggccaggctggacctcggcatccccggcgacgtgcccaagtacgacatcatcttcgtgaacgtcaggaccccatacaagtaccaccattaccagcagtgcgaggaccacgccatcaagctgagcatgctgaccaagaaggcctgcctgcacctgaaccccggaggcacctgcgtgagcatcggctacggctacgccgacagggccagcgagagcatcattggcgccatcgccaggctgttcaagttcagcagggtgtgcaaacccaagagcagcctggaggaaaccgaggtgctgttcgtgttcatcggctacgaccggaaggccaggacccacaacccctacaagctgagcagcaccctgacaaacatctacaccggcagcaggctgcacgaggccggctgcgcccccagctaccacgtggtcaggggcgatatcgccaccgccaccgagggcgtgatcatcaacgctgccaacagcaagggccagcccggaggcggagtgtgcggcgccctgtacaagaagttccccgagagcttcgacctgcagcccatcgaggtgggcaaggccaggctggtgaagggcgccgctaagcacatcatccacgccgtgggccccaacttcaacaaggtgagcgaggtggaaggcgacaagcagctggccgaagcctacgagagcatcgccaagatcgtgaacgacaataactacaagagcgtggccatcccactgctcagcaccggcatcttcagcggcaacaaggacaggctgacccagagcctgaaccacctgctcaccgccctggacaccaccgatgccgacgtggccatctactgcagggacaagaagtgggagatgaccctgaaggaggccgtggccaggcgggaggccgtggaagagatctgcatcagcgacgactccagcgtgaccgagcccgacgccgagctggtgagggtgcaccccaagagctccctggccggcaggaagggctacagcaccagcgacggcaagaccttcagctacctggagggcaccaagttccaccaggccgctaaggacatcgccgagatcaacgctatgtggcccgtggccaccgaggccaacgagcaggtgtgcatgtacatcctgggcgagagcatgtccagcatcaggagcaagtgccccgtggaggaaagcgaggccagcacaccacccagcaccctgccctgcctgtgcatccacgctatgacacccgagagggtgcagcggctgaaggccagcaggcccgagcagatcaccgtgtgcagctccttcccactgcccaagtacaggatcaccggcgtgcagaagatccagtgcagccagcccatcctgttcagcccaaaggtgcccgcctacatccaccccaggaagtacctggtggagaccccacccgtggacgagacacccgagccaagcgccgagaaccagagcaccgagggcacacccgagcagccacccctgatcaccgaggacgagacaaggacccggaccccagagcccatcattatcgaggaagaggaagaggacagcatcagcctgctgagcgacggccccacccaccaggtgctgcaggtggaggccgacatccacggcccacccagcgtgtccagctccagctggagcatcccacacgccagcgacttcgacgtggacagcctgagcatcctggacaccctggagggcgccagcgtgacctccggcgccaccagcgccgagaccaacagctacttcgccaagagcatggagttcctggccaggcccgtgccagctcccaggaccgtgttcaggaacccaccccacccagctcccaggaccaggaccccaagcctggctcccagcagggcctgcagcaggaccagcctggtgagcaccccacccggcgtgaacagggtgatcaccagggaggaactggaggccctgacacccagcaggacccccagcaggtccgtgagcaggactagtctggtgtccaacccacccggcgtgaacagggtgatcaccagggaggaattcgaggccttcgtggcccagcaacagagacggttcgacgccggcgcctacatcttcagcagcgacaccggccagggacacctgcagcaaaagagcgtgaggcagaccgtgctgagcgaggtggtgctggagaggaccgagctggaaatcagctacgcccccaggctggaccaggagaaggaggaactgctcaggaagaaactgcagctgaaccccaccccagccaacaggagcaggtaccagagcaggaaggtggagaacatgaaggccatcaccgccaggcggatcctgcagggcctgggacactacctgaaggccgagggcaaggtggagtgctacaggaccctgcaccccgtgccactgtacagctccagcgtgaacagggccttctccagccccaaggtggccgtggaggcctgcaacgctatgctgaaggagaacttccccaccgtggccagctactgcatcatccccgagtacgacgcctacctggacatggtggacggcgccagctgctgcctggacaccgccagcttctgccccgccaagctgaggagcttccccaagaaacacagctacctggagcccaccatcaggagcgccgtgcccagcgccatccagaacaccctgcagaacgtgctggccgctgccaccaagaggaactgcaacgtgacccagatgagggagctgcccgtgctggacagcgctgccttcaacgtggagtgcttcaagaaatacgcctgcaacaacgagtactgggagaccttcaaggagaaccccatcaggctgaccgaagagaacgtggtgaactacatcaccaagctgaagggccccaaggccgctgccctgttcgctaagacccacaacctgaacatgctgcaggacatcccaatggacaggttcgtgatggacctgaagagggacgtgaaggtgacacccggcaccaagcacaccgaggagaggcccaaggtgcaggtgatccaggccgctgacccactggccaccgcctacctgtgcggcatccacagggagctggtgaggcggctgaacgccgtgctgctgcccaacatccacaccctgttcgacatgagcgccgaggacttcgacgccatcatcgccgagcacttccagcccggcgactgcgtgctggagaccgacatcgccagcttcgacaagagcgaggatgacgctatggccctgaccgctctgatgatcctggaggacctgggcgtggacgccgagctgctcaccctgatcgaggctgccttcggcgagatcagctccatccacctgcccaccaagaccaagttcaagttcggcgctatgatgaaaagcggaatgttcctgaccctgttcgtgaacaccgtgatcaacattgtgatcgccagcagggtgctgcgggagaggctgaccggcagcccctgcgctgccttcatcggcgacgacaacatcgtgaagggcgtgaaaagcgacaagctgatggccgacaggtgcgccacctggctgaacatggaggtgaagatcatcgacgccgtggtgggcgagaaggccccctacttctgcggcggattcatcctgtgcgacagcgtgaccggcaccgcctgcagggtggccgaccccctgaagaggctgttcaagctgggcaagccactggccgctgacgatgagcacgacgatgacaggcggagggccctgcacgaggaaagcaccaggtggaacagggtgggcatcctgagcgagctgtgcaaggccgtggagagcaggtacgagaccgtgggcaccagcatcatcgtgatggctatgaccacactggccagctccgtcaagagcttctcctacctgaggggggcccctataactctctacggctaacctgaatggactacgacatagtctagtccgccaaggccgccaccatgagagtgacagcccctagaaccttactgcttctgctttggggagctgttgctctgacagagacatgggctggatctctgagcgaggtgaccggccagggcctgtgcatcggcgccgtgcccaagacccaccaggtgctgtgcaacaccacccagaagaccagcgacggcagctactacctggccgctcccaccggcaccacctgggcctgcagcaccggcctgaccccttgcatcagcaccaccatcctgaacctgaccaccgactactgcgtgctggtggagctgtggcccagggtgacctaccacagccccagctacgcctaccaccagttcgagaggagggccaagtacaagagggagcccgtgagcctgaccctggccctgctgctgggcggcctgacaatgggcggcatcgccgccggcgtgggcaccggcaccaccgccctggtggccacccagcagttccagcagctgcaggccgccatgcacgacgacctgaaggaggtggagaagtccatcaccaacctggagaagtccctgaccagcctgagcgaggtggtgctgcagaacaggaggggcctggacctgctgttcctgaaggagggcggcctgtgcgccgccctgaaggaggagtgctgcctgtacgccgaccacaccggcctggtgatcgtgggcattgtcgctggcctggccgtcctcgccgtggtggtgattggagctgtggtcgcagctgttatgtgcagaagaaagtcatccggcggaaagggaggctcctactctcaggctgcttctgctacagtgcctagagctcttatgtgtttatctcagctgtaaactcgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaatctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 106 - RNA, 8416 nt, synthetic construct
  misc_feature 1..8416: note = Synthetic polynucleotide
  source 1..8416: mol_type = other RNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgccgtcgacggccccaccagcctgtaccaccaggccaacaagggcgtgagggtggcctactggatcggcttcgacaccacacccttcatgttcaagaacctggccggcgcctaccccagctacagcaccaactgggccgacgagaccgtgctgaccgccaggaacatcggcctgtgcagcagcgacgtgatggagaggagccggagaggcatgagcatcctgaggaagaaatacctgaagcccagcaacaacgtgctgttcagcgtgggcagcaccatctaccacgagaagagggacctgctcaggagctggcacctgcccagcgtgttccacctgaggggcaagcagaactacacctgcaggtgcgagaccatcgtgagctgcgacggctacgtggtgaagaggatcgccatcagccccggcctgtacggcaagcccagcggctacgccgctacaatgcacagggagggcttcctgtgctgcaaggtgaccgacaccctgaacggcgagagggtgagcttccccgtgtgcacctacgtgcccgccaccctgtgcgaccagatgaccggcatcctggccaccgacgtgagcgccgacgacgcccagaagctgctcgtgggcctgaaccagaggatcgtggtcaacggcaggacccagaggaacaccaacacaatgaagaactacctgctgcccgtggtggcccaggctttcgccaggtgggccaaggagtacaaggaggaccaggaagacgagaggcccctgggcctgagggacaggcagctggtgatgggctgctgctgggccttcaggcggcacaagatcaccagcatctacaagaggcccgacacccagaccatcatcaaggtgaacagcgacttccacagcttcgtgctgcccaggatcggcagcaacaccctggagatcggcctgaggacccggatcaggaagatgctggaggaacacaaggagcccagcccactgatcaccgccgaggacgtgcaggaggccaagtgcgctgccgacgaggccaaggaggtgagggaggccgaggaactgagggccgccctgccacccctggctgccgacgtggaggaacccaccctggaagccgacgtggacctgatgctgcaggaggccggcgccggaagcgtggagacacccaggggcctgatcaaggtgaccagctacgacggcgaggacaagatcggcagctacgccgtgctgagcccacaggccgtgctgaagtccgagaagctgagctgcatccacccactggccgagcaggtgatcgtgatcacccacagcggcaggaagggcaggtacgccgtggagccctaccacggcaaggtggtcgtgcccgagggccacgccatccccgtgcaggacttccaggccctgagcgagagcgccaccatcgtgtacaacgagagggagttcgtgaacaggtacctgcaccatatcgccacccacggcggagccctgaacaccgacgaggaatactacaagaccgtgaagcccagcgagcacgacggcgagtacctgtacgacatcgacaggaagcagtgcgtgaagaaagagctggtgaccggcctgggactgaccggcgagctggtggacccacccttccacgagttcgcctacgagagcctgaggaccagacccgccgctccctaccaggtgcccaccatcggcgtgtacggcgtgcccggcagcggaaagagcggcatcatcaagagcgccgtgaccaagaaagacctggtggtcagcgccaagaaagagaactgcgccgagatcatcagggacgtgaagaagatgaaaggcctggacgtgaacgcgcgcaccgtggacagcgtgctgctgaacggctgcaagcaccccgtggagaccctgtacatcgacgaggccttcgcttgccacgccggcaccctgagggccctgatcgccatcatcaggcccaagaaagccgtgctgtgcggcgaccccaagcagtgcggcttcttcaacatgatgtgcctgaaggtgcacttcaaccacgagatctgcacccaggtgttccacaagagcatcagcaggcggtgcaccaagagcgtgaccagcgtcgtgagcaccctgttctacgacaagaaaatgaggaccaccaaccccaaggagaccaaaatcgtgatcgacaccacaggcagcaccaagcccaagcaggacgacctgatcctgacctgcttcaggggctgggtgaagcagctgcagatcgactacaagggcaacgagatcatgaccgccgctgccagccagggcctgaccaggaagggcgtgtacgccgtgaggtacaaggtgaacgagaacccactgtacgctcccaccagcgagcacgtgaacgtgctgctgaccaggaccgaggacaggatcgtgtggaagaccctggccggcgacccctggatcaagaccctgaccgccaagtaccccggcaacttcaccgccaccatcgaagagtggcaggccgagcacgacgccatcatgaggcacatcctggagaggcccgaccccaccgacgtgttccagaacaaggccaacgtgtgctgggccaaggccctggtgcccgtgctgaagaccgccggcatcgacatgaccacagagcagtggaacaccgtggactacttcgagaccgacaaggcccacagcgccgagatcgtgctgaaccagctgtgcgtgaggttcttcggcctggacctggacagcggcctgttcagcgcccccaccgtgccactgagcatcaggaacaaccactgggacaacagccccagcccaaacatgtacggcctgaacaaggaggtggtcaggcagctgagcaggcggtacccacagctgcccagggccgtggccaccggcagggtgtacgacatgaacaccggcaccctgaggaactacgaccccaggatcaacctggtgcccgtgaacaggcggctgccccacgccctggtgctgcaccacaacgagcacccacagagcgacttcagctccttcgtgagcaagctgaaaggcaggaccgtgctggtcgtgggcgagaagctgagcgtgcccggcaagatggtggactggctgagcgacaggcccgaggccaccttccgggccaggctggacctcggcatccccggcgacgtgcccaagtacgacatcatcttcgtgaacgtcaggaccccatacaagtaccaccattaccagcagtgcgaggaccacgccatcaagctgagcatgctgaccaagaaggcctgcctgcacctgaaccccggaggcacctgcgtgagcatcggctacggctacgccgacagggccagcgagagcatcattggcgccatcgccaggctgttcaagttcagcagggtgtgcaaacccaagagcagcctggaggaaaccgaggtgctgttcgtgttcatcggctacgaccggaaggccaggacccacaacccctacaagctgagcagcaccctgacaaacatctacaccggcagcaggctgcacgaggccggctgcgcccccagctaccacgtggtcaggggcgatatcgccaccgccaccgagggcgtgatcatcaacgctgccaacagcaagggccagcccggaggcggagtgtgcggcgccctgtacaagaagttccccgagagcttcgacctgcagcccatcgaggtgggcaaggccaggctggtgaagggcgccgctaagcacatcatccacgccgtgggccccaacttcaacaaggtgagcgaggtggaaggcgacaagcagctggccgaagcctacgagagcatcgccaagatcgtgaacgacaataactacaagagcgtggccatcccactgctcagcaccggcatcttcagcggcaacaaggacaggctgacccagagcctgaaccacctgctcaccgccctggacaccaccgatgccgacgtggccatctactgcagggacaagaagtgggagatgaccctgaaggaggccgtggccaggcgggaggccgtggaagagatctgcatcagcgacgactccagcgtgaccgagcccgacgccgagctggtgagggtgcaccccaagagctccctggccggcaggaagggctacagcaccagcgacggcaagaccttcagctacctggagggcaccaagttccaccaggccgctaaggacatcgccgagatcaacgctatgtggcccgtggccaccgaggccaacgagcaggtgtgcatgtacatcctgggcgagagcatgtccagcatcaggagcaagtgccccgtggaggaaagcgaggccagcacaccacccagcaccctgccctgcctgtgcatccacgctatgacacccgagagggtgcagcggctgaaggccagcaggcccgagcagatcaccgtgtgcagctccttcccactgcccaagtacaggatcaccggcgtgcagaagatccagtgcagccagcccatcctgttcagcccaaaggtgcccgcctacatccaccccaggaagtacctggtggagaccccacccgtggacgagacacccgagccaagcgccgagaaccagagcaccgagggcacacccgagcagccacccctgatcaccgaggacgagacaaggacccggaccccagagcccatcattatcgaggaagaggaagaggacagcatcagcctgctgagcgacggccccacccaccaggtgctgcaggtggaggccgacatccacggcccacccagcgtgtccagctccagctggagcatcccacacgccagcgacttcgacgtggacagcctgagcatcctggacaccctggagggcgccagcgtgacctccggcgccaccagcgccgagaccaacagctacttcgccaagagcatggagttcctggccaggcccgtgccagctcccaggaccgtgttcaggaacccaccccacccagctcccaggaccaggaccccaagcctggctcccagcagggcctgcagcaggaccagcctggtgagcaccccacccggcgtgaacagggtgatcaccagggaggaactggaggccctgacacccagcaggacccccagcaggtccgtgagcaggactagtctggtgtccaacccacccggcgtgaacagggtgatcaccagggaggaattcgaggccttcgtggcccagcaacagagacggttcgacgccggcgcctacatcttcagcagcgacaccggccagggacacctgcagcaaaagagcgtgaggcagaccgtgctgagcgaggtggtgctggagaggaccgagctggaaatcagctacgcccccaggctggaccaggagaaggaggaactgctcaggaagaaactgcagctgaaccccaccccagccaacaggagcaggtaccagagcaggaaggtggagaacatgaaggccatcaccgccaggcggatcctgcagggcctgggacactacctgaaggccgagggcaaggtggagtgctacaggaccctgcaccccgtgccactgtacagctccagcgtgaacagggccttctccagccccaaggtggccgtggaggcctgcaacgctatgctgaaggagaacttccccaccgtggccagctactgcatcatccccgagtacgacgcctacctggacatggtggacggcgccagctgctgcctggacaccgccagcttctgccccgccaagctgaggagcttccccaagaaacacagctacctggagcccaccatcaggagcgccgtgcccagcgccatccagaacaccctgcagaacgtgctggccgctgccaccaagaggaactgcaacgtgacccagatgagggagctgcccgtgctggacagcgctgccttcaacgtggagtgcttcaagaaatacgcctgcaacaacgagtactgggagaccttcaaggagaaccccatcaggctgaccgaagagaacgtggtgaactacatcaccaagctgaagggccccaaggccgctgccctgttcgctaagacccacaacctgaacatgctgcaggacatcccaatggacaggttcgtgatggacctgaagagggacgtgaaggtgacacccggcaccaagcacaccgaggagaggcccaaggtgcaggtgatccaggccgctgacccactggccaccgcctacctgtgcggcatccacagggagctggtgaggcggctgaacgccgtgctgctgcccaacatccacaccctgttcgacatgagcgccgaggacttcgacgccatcatcgccgagcacttccagcccggcgactgcgtgctggagaccgacatcgccagcttcgacaagagcgaggatgacgctatggccctgaccgctctgatgatcctggaggacctgggcgtggacgccgagctgctcaccctgatcgaggctgccttcggcgagatcagctccatccacctgcccaccaagaccaagttcaagttcggcgctatgatgaaaagcggaatgttcctgaccctgttcgtgaacaccgtgatcaacattgtgatcgccagcagggtgctgcgggagaggctgaccggcagcccctgcgctgccttcatcggcgacgacaacatcgtgaagggcgtgaaaagcgacaagctgatggccgacaggtgcgccacctggctgaacatggaggtgaagatcatcgacgccgtggtgggcgagaaggccccctacttctgcggcggattcatcctgtgcgacagcgtgaccggcaccgcctgcagggtggccgaccccctgaagaggctgttcaagctgggcaagccactggccgctgacgatgagcacgacgatgacaggcggagggccctgcacgaggaaagcaccaggtggaacagggtgggcatcctgagcgagctgtgcaaggccgtggagagcaggtacgagaccgtgggcaccagcatcatcgtgatggctatgaccacactggccagctccgtcaagagcttctcctacctgaggggggcccctataactctctacggctaacctgaatggactacgacatagtctagtccgccaaggccgccaccatgagagtgacagcccctagaaccttactgcttctgctttggggagctgttgctctgacagagacatgggctggatcttaccacagccccagctacgcctaccaccagttcgagagggggggaggaggctccgggggaggaggctccctgaagatcagccaggccgtgcacgccgcccacgccgagatcaacgaggccggccgggaggtgatcgtgggcattgtcgctggcctggccgtcctcgccgtggtggtgattggagctgtggtcgcagctgttatgtgcagaagaaagtcatccggcggaaagggaggctcctactctcaggctgcttctgctacagtgcctagagctcttatgtgtttatctcagctgtaaactcgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaatctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 107 - RNA, 8914 nt, synthetic construct
  misc_feature 1..8914: note = Synthetic polynucleotide
  source 1..8914: mol_type = other RNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgccgtcgacggccccaccagcctgtaccaccaggccaacaagggcgtgagggtggcctactggatcggcttcgacaccacacccttcatgttcaagaacctggccggcgcctaccccagctacagcaccaactgggccgacgagaccgtgctgaccgccaggaacatcggcctgtgcagcagcgacgtgatggagaggagccggagaggcatgagcatcctgaggaagaaatacctgaagcccagcaacaacgtgctgttcagcgtgggcagcaccatctaccacgagaagagggacctgctcaggagctggcacctgcccagcgtgttccacctgaggggcaagcagaactacacctgcaggtgcgagaccatcgtgagctgcgacggctacgtggtgaagaggatcgccatcagccccggcctgtacggcaagcccagcggctacgccgctacaatgcacagggagggcttcctgtgctgcaaggtgaccgacaccctgaacggcgagagggtgagcttccccgtgtgcacctacgtgcccgccaccctgtgcgaccagatgaccggcatcctggccaccgacgtgagcgccgacgacgcccagaagctgctcgtgggcctgaaccagaggatcgtggtcaacggcaggacccagaggaacaccaacacaatgaagaactacctgctgcccgtggtggcccaggctttcgccaggtgggccaaggagtacaaggaggaccaggaagacgagaggcccctgggcctgagggacaggcagctggtgatgggctgctgctgggccttcaggcggcacaagatcaccagcatctacaagaggcccgacacccagaccatcatcaaggtgaacagcgacttccacagcttcgtgctgcccaggatcggcagcaacaccctggagatcggcctgaggacccggatcaggaagatgctggaggaacacaaggagcccagcccactgatcaccgccgaggacgtgcaggaggccaagtgcgctgccgacgaggccaaggaggtgagggaggccgaggaactgagggccgccctgccacccctggctgccgacgtggaggaacccaccctggaagccgacgtggacctgatgctgcaggaggccggcgccggaagcgtggagacacccaggggcctgatcaaggtgaccagctacgacggcgaggacaagatcggcagctacgccgtgctgagcccacaggccgtgctgaagtccgagaagctgagctgcatccacccactggccgagcaggtgatcgtgatcacccacagcggcaggaagggcaggtacgccgtggagccctaccacggcaaggtggtcgtgcccgagggccacgccatccccgtgcaggacttccaggccctgagcgagagcgccaccatcgtgtacaacgagagggagttcgtgaacaggtacctgcaccatatcgccacccacggcggagccctgaacaccgacgaggaatactacaagaccgtgaagcccagcgagcacgacggcgagtacctgtacgacatcgacaggaagcagtgcgtgaagaaagagctggtgaccggcctgggactgaccggcgagctggtggacccacccttccacgagttcgcctacgagagcctgaggaccagacccgccgctccctaccaggtgcccaccatcggcgtgtacggcgtgcccggcagcggaaagagcggcatcatcaagagcgccgtgaccaagaaagacctggtggtcagcgccaagaaagagaactgcgccgagatcatcagggacgtgaagaagatgaaaggcctggacgtgaacgcgcgcaccgtggacagcgtgctgctgaacggctgcaagcaccccgtggagaccctgtacatcgacgaggccttcgcttgccacgccggcaccctgagggccctgatcgccatcatcaggcccaagaaagccgtgctgtgcggcgaccccaagcagtgcggcttcttcaacatgatgtgcctgaaggtgcacttcaaccacgagatctgcacccaggtgttccacaagagcatcagcaggcggtgcaccaagagcgtgaccagcgtcgtgagcaccctgttctacgacaagaaaatgaggaccaccaaccccaaggagaccaaaatcgtgatcgacaccacaggcagcaccaagcccaagcaggacgacctgatcctgacctgcttcaggggctgggtgaagcagctgcagatcgactacaagggcaacgagatcatgaccgccgctgccagccagggcctgaccaggaagggcgtgtacgccgtgaggtacaaggtgaacgagaacccactgtacgctcccaccagcgagcacgtgaacgtgctgctgaccaggaccgaggacaggatcgtgtggaagaccctggccggcgacccctggatcaagaccctgaccgccaagtaccccggcaacttcaccgccaccatcgaagagtggcaggccgagcacgacgccatcatgaggcacatcctggagaggcccgaccccaccgacgtgttccagaacaaggccaacgtgtgctgggccaaggccctggtgcccgtgctgaagaccgccggcatcgacatgaccacagagcagtggaacaccgtggactacttcgagaccgacaaggcccacagcgccgagatcgtgctgaaccagctgtgcgtgaggttcttcggcctggacctggacagcggcctgttcagcgcccccaccgtgccactgagcatcaggaacaaccactgggacaacagccccagcccaaacatgtacggcctgaacaaggaggtggtcaggcagctgagcaggcggtacccacagctgcccagggccgtggccaccggcagggtgtacgacatgaacaccggcaccctgaggaactacgaccccaggatcaacctggtgcccgtgaacaggcggctgccccacgccctggtgctgcaccacaacgagcacccacagagcgacttcagctccttcgtgagcaagctgaaaggcaggaccgtgctggtcgtgggcgagaagctgagcgtgcccggcaagatggtggactggctgagcgacaggcccgaggccaccttccgggccaggctggacctcggcatccccggcgacgtgcccaagtacgacatcatcttcgtgaacgtcaggaccccatacaagtaccaccattaccagcagtgcgaggaccacgccatcaagctgagcatgctgaccaagaaggcctgcctgcacctgaaccccggaggcacctgcgtgagcatcggctacggctacgccgacagggccagcgagagcatcattggcgccatcgccaggctgttcaagttcagcagggtgtgcaaacccaagagcagcctggaggaaaccgaggtgctgttcgtgttcatcggctacgaccggaaggccaggacccacaacccctacaagctgagcagcaccctgacaaacatctacaccggcagcaggctgcacgaggccggctgcgcccccagctaccacgtggtcaggggcgatatcgccaccgccaccgagggcgtgatcatcaacgctgccaacagcaagggccagcccggaggcggagtgtgcggcgccctgtacaagaagttccccgagagcttcgacctgcagcccatcgaggtgggcaaggccaggctggtgaagggcgccgctaagcacatcatccacgccgtgggccccaacttcaacaaggtgagcgaggtggaaggcgacaagcagctggccgaagcctacgagagcatcgccaagatcgtgaacgacaataactacaagagcgtggccatcccactgctcagcaccggcatcttcagcggcaacaaggacaggctgacccagagcctgaaccacctgctcaccgccctggacaccaccgatgccgacgtggccatctactgcagggacaagaagtgggagatgaccctgaaggaggccgtggccaggcgggaggccgtggaagagatctgcatcagcgacgactccagcgtgaccgagcccgacgccgagctggtgagggtgcaccccaagagctccctggccggcaggaagggctacagcaccagcgacggcaagaccttcagctacctggagggcaccaagttccaccaggccgctaaggacatcgccgagatcaacgctatgtggcccgtggccaccgaggccaacgagcaggtgtgcatgtacatcctgggcgagagcatgtccagcatcaggagcaagtgccccgtggaggaaagcgaggccagcacaccacccagcaccctgccctgcctgtgcatccacgctatgacacccgagagggtgcagcggctgaaggccagcaggcccgagcagatcaccgtgtgcagctccttcccactgcccaagtacaggatcaccggcgtgcagaagatccagtgcagccagcccatcctgttcagcccaaaggtgcccgcctacatccaccccaggaagtacctggtggagaccccacccgtggacgagacacccgagccaagcgccgagaaccagagcaccgagggcacacccgagcagccacccctgatcaccgaggacgagacaaggacccggaccccagagcccatcattatcgaggaagaggaagaggacagcatcagcctgctgagcgacggccccacccaccaggtgctgcaggtggaggccgacatccacggcccacccagcgtgtccagctccagctggagcatcccacacgccagcgacttcgacgtggacagcctgagcatcctggacaccctggagggcgccagcgtgacctccggcgccaccagcgccgagaccaacagctacttcgccaagagcatggagttcctggccaggcccgtgccagctcccaggaccgtgttcaggaacccaccccacccagctcccaggaccaggaccccaagcctggctcccagcagggcctgcagcaggaccagcctggtgagcaccccacccggcgtgaacagggtgatcaccagggaggaactggaggccctgacacccagcaggacccccagcaggtccgtgagcaggactagtctggtgtccaacccacccggcgtgaacagggtgatcaccagggaggaattcgaggccttcgtggcccagcaacagagacggttcgacgccggcgcctacatcttcagcagcgacaccggccagggacacctgcagcaaaagagcgtgaggcagaccgtgctgagcgaggtggtgctggagaggaccgagctggaaatcagctacgcccccaggctggaccaggagaaggaggaactgctcaggaagaaactgcagctgaaccccaccccagccaacaggagcaggtaccagagcaggaaggtggagaacatgaaggccatcaccgccaggcggatcctgcagggcctgggacactacctgaaggccgagggcaaggtggagtgctacaggaccctgcaccccgtgccactgtacagctccagcgtgaacagggccttctccagccccaaggtggccgtggaggcctgcaacgctatgctgaaggagaacttccccaccgtggccagctactgcatcatccccgagtacgacgcctacctggacatggtggacggcgccagctgctgcctggacaccgccagcttctgccccgccaagctgaggagcttccccaagaaacacagctacctggagcccaccatcaggagcgccgtgcccagcgccatccagaacaccctgcagaacgtgctggccgctgccaccaagaggaactgcaacgtgacccagatgagggagctgcccgtgctggacagcgctgccttcaacgtggagtgcttcaagaaatacgcctgcaacaacgagtactgggagaccttcaaggagaaccccatcaggctgaccgaagagaacgtggtgaactacatcaccaagctgaagggccccaaggccgctgccctgttcgctaagacccacaacctgaacatgctgcaggacatcccaatggacaggttcgtgatggacctgaagagggacgtgaaggtgacacccggcaccaagcacaccgaggagaggcccaaggtgcaggtgatccaggccgctgacccactggccaccgcctacctgtgcggcatccacagggagctggtgaggcggctgaacgccgtgctgctgcccaacatccacaccctgttcgacatgagcgccgaggacttcgacgccatcatcgccgagcacttccagcccggcgactgcgtgctggagaccgacatcgccagcttcgacaagagcgaggatgacgctatggccctgaccgctctgatgatcctggaggacctgggcgtggacgccgagctgctcaccctgatcgaggctgccttcggcgagatcagctccatccacctgcccaccaagaccaagttcaagttcggcgctatgatgaaaagcggaatgttcctgaccctgttcgtgaacaccgtgatcaacattgtgatcgccagcagggtgctgcgggagaggctgaccggcagcccctgcgctgccttcatcggcgacgacaacatcgtgaagggcgtgaaaagcgacaagctgatggccgacaggtgcgccacctggctgaacatggaggtgaagatcatcgacgccgtggtgggcgagaaggccccctacttctgcggcggattcatcctgtgcgacagcgtgaccggcaccgcctgcagggtggccgaccccctgaagaggctgttcaagctgggcaagccactggccgctgacgatgagcacgacgatgacaggcggagggccctgcacgaggaaagcaccaggtggaacagggtgggcatcctgagcgagctgtgcaaggccgtggagagcaggtacgagaccgtgggcaccagcatcatcgtgatggctatgaccacactggccagctccgtcaagagcttctcctacctgaggggggcccctataactctctacggctaacctgaatggactacgacatagtctagtccgccaaggccgccaccatgagagtgacagcccctagaaccttactgcttctgctttggggagctgttgctctgacagagacatgggctggatctctgagcgaggtgaccggccagggcctgtgcatcggcgccgtgcccaagacccaccaggtgctgtgcaacaccacccagaagaccagcgacggcagctactacctggccgctcccaccggcaccacctgggcctgcagcaccggcctgaccccttgcatcagcaccaccatcctgaacctgaccaccgactactgcgtgctggtggagctgtggcccagggtgacctaccacagccccagctacgcctaccaccagttcgagaggagggccaagtacaagagggagcccgtgagcctgaccctggccctgctgctgggcggcctgacaatgggcggcatcgccgccggcgtgggcaccggcaccaccgccctggtggccacccagcagttccagcagctgcaggccgccatgcacgacgacctgaaggaggtggagaagtccatcaccaacctggagaagtccctgaccagcctgagcgaggtggtgctgcagaacaggaggggcctggacctgctgttcctgaaggagggcggcctgtgcgccgccctgaaggaggagtgctgcctgtacgccgaccacaccggcctggtgatcgtgggcattgtcgctggcctggccgtcctcgccgtggtggtgattggagctgtggtcgcagctgttatgtgcagaagaaagtcatccggcggaaagggaggctcctactctcaggctgcttctgctacagtgcctagagctcttatgtgtttatctcagctgggcggcggaggcagcgactacaaggacgacgatgacaagtaaactcgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaatctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 108 - RNA, 8455 nt, synthetic construct
  misc_feature 1..8455: note = Synthetic polynucleotide
  source 1..8455: mol_type = other RNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgccgtcgacggccccaccagcctgtaccaccaggccaacaagggcgtgagggtggcctactggatcggcttcgacaccacacccttcatgttcaagaacctggccggcgcctaccccagctacagcaccaactgggccgacgagaccgtgctgaccgccaggaacatcggcctgtgcagcagcgacgtgatggagaggagccggagaggcatgagcatcctgaggaagaaatacctgaagcccagcaacaacgtgctgttcagcgtgggcagcaccatctaccacgagaagagggacctgctcaggagctggcacctgcccagcgtgttccacctgaggggcaagcagaactacacctgcaggtgcgagaccatcgtgagctgcgacggctacgtggtgaagaggatcgccatcagccccggcctgtacggcaagcccagcggctacgccgctacaatgcacagggagggcttcctgtgctgcaaggtgaccgacaccctgaacggcgagagggtgagcttccccgtgtgcacctacgtgcccgccaccctgtgcgaccagatgaccggcatcctggccaccgacgtgagcgccgacgacgcccagaagctgctcgtgggcctgaaccagaggatcgtggtcaacggcaggacccagaggaacaccaacacaatgaagaactacctgctgcccgtggtggcccaggctttcgccaggtgggccaaggagtacaaggaggaccaggaagacgagaggcccctgggcctgagggacaggcagctggtgatgggctgctgctgggccttcaggcggcacaagatcaccagcatctacaagaggcccgacacccagaccatcatcaaggtgaacagcgacttccacagcttcgtgctgcccaggatcggcagcaacaccctggagatcggcctgaggacccggatcaggaagatgctggaggaacacaaggagcccagcccactgatcaccgccgaggacgtgcaggaggccaagtgcgctgccgacgaggccaaggaggtgagggaggccgaggaactgagggccgccctgccacccctggctgccgacgtggaggaacccaccctggaagccgacgtggacctgatgctgcaggaggccggcgccggaagcgtggagacacccaggggcctgatcaaggtgaccagctacgacggcgaggacaagatcggcagctacgccgtgctgagcccacaggccgtgctgaagtccgagaagctgagctgcatccacccactggccgagcaggtgatcgtgatcacccacagcggcaggaagggcaggtacgccgtggagccctaccacggcaaggtggtcgtgcccgagggccacgccatccccgtgcaggacttccaggccctgagcgagagcgccaccatcgtgtacaacgagagggagttcgtgaacaggtacctgcaccatatcgccacccacggcggagccctgaacaccgacgaggaatactacaagaccgtgaagcccagcgagcacgacggcgagtacctgtacgacatcgacaggaagcagtgcgtgaagaaagagctggtgaccggcctgggactgaccggcgagctggtggacccacccttccacgagttcgcctacgagagcctgaggaccagacccgccgctccctaccaggtgcccaccatcggcgtgtacggcgtgcccggcagcggaaagagcggcatcatcaagagcgccgtgaccaagaaagacctggtggtcagcgccaagaaagagaactgcgccgagatcatcagggacgtgaagaagatgaaaggcctggacgtgaacgcgcgcaccgtggacagcgtgctgctgaacggctgcaagcaccccgtggagaccctgtacatcgacgaggccttcgcttgccacgccggcaccctgagggccctgatcgccatcatcaggcccaagaaagccgtgctgtgcggcgaccccaagcagtgcggcttcttcaacatgatgtgcctgaaggtgcacttcaaccacgagatctgcacccaggtgttccacaagagcatcagcaggcggtgcaccaagagcgtgaccagcgtcgtgagcaccctgttctacgacaagaaaatgaggaccaccaaccccaaggagaccaaaatcgtgatcgacaccacaggcagcaccaagcccaagcaggacgacctgatcctgacctgcttcaggggctgggtgaagcagctgcagatcgactacaagggcaacgagatcatgaccgccgctgccagccagggcctgaccaggaagggcgtgtacgccgtgaggtacaaggtgaacgagaacccactgtacgctcccaccagcgagcacgtgaacgtgctgctgaccaggaccgaggacaggatcgtgtggaagaccctggccggcgacccctggatcaagaccctgaccgccaagtaccccggcaacttcaccgccaccatcgaagagtggcaggccgagcacgacgccatcatgaggcacatcctggagaggcccgaccccaccgacgtgttccagaacaaggccaacgtgtgctgggccaaggccctggtgcccgtgctgaagaccgccggcatcgacatgaccacagagcagtggaacaccgtggactacttcgagaccgacaaggcccacagcgccgagatcgtgctgaaccagctgtgcgtgaggttcttcggcctggacctggacagcggcctgttcagcgcccccaccgtgccactgagcatcaggaacaaccactgggacaacagccccagcccaaacatgtacggcctgaacaaggaggtggtcaggcagctgagcaggcggtacccacagctgcccagggccgtggccaccggcagggtgtacgacatgaacaccggcaccctgaggaactacgaccccaggatcaacctggtgcccgtgaacaggcggctgccccacgccctggtgctgcaccacaacgagcacccacagagcgacttcagctccttcgtgagcaagctgaaaggcaggaccgtgctggtcgtgggcgagaagctgagcgtgcccggcaagatggtggactggctgagcgacaggcccgaggccaccttccgggccaggctggacctcggcatccccggcgacgtgcccaagtacgacatcatcttcgtgaacgtcaggaccccatacaagtaccaccattaccagcagtgcgaggaccacgccatcaagctgagcatgctgaccaagaaggcctgcctgcacctgaaccccggaggcacctgcgtgagcatcggctacggctacgccgacagggccagcgagagcatcattggcgccatcgccaggctgttcaagttcagcagggtgtgcaaacccaagagcagcctggaggaaaccgaggtgctgttcgtgttcatcggctacgaccggaaggccaggacccacaacccctacaagctgagcagcaccctgacaaacatctacaccggcagcaggctgcacgaggccggctgcgcccccagctaccacgtggtcaggggcgatatcgccaccgccaccgagggcgtgatcatcaacgctgccaacagcaagggccagcccggaggcggagtgtgcggcgccctgtacaagaagttccccgagagcttcgacctgcagcccatcgaggtgggcaaggccaggctggtgaagggcgccgctaagcacatcatccacgccgtgggccccaacttcaacaaggtgagcgaggtggaaggcgacaagcagctggccgaagcctacgagagcatcgccaagatcgtgaacgacaataactacaagagcgtggccatcccactgctcagcaccggcatcttcagcggcaacaaggacaggctgacccagagcctgaaccacctgctcaccgccctggacaccaccgatgccgacgtggccatctactgcagggacaagaagtgggagatgaccctgaaggaggccgtggccaggcgggaggccgtggaagagatctgcatcagcgacgactccagcgtgaccgagcccgacgccgagctggtgagggtgcaccccaagagctccctggccggcaggaagggctacagcaccagcgacggcaagaccttcagctacctggagggcaccaagttccaccaggccgctaaggacatcgccgagatcaacgctatgtggcccgtggccaccgaggccaacgagcaggtgtgcatgtacatcctgggcgagagcatgtccagcatcaggagcaagtgccccgtggaggaaagcgaggccagcacaccacccagcaccctgccctgcctgtgcatccacgctatgacacccgagagggtgcagcggctgaaggccagcaggcccgagcagatcaccgtgtgcagctccttcccactgcccaagtacaggatcaccggcgtgcagaagatccagtgcagccagcccatcctgttcagcccaaaggtgcccgcctacatccaccccaggaagtacctggtggagaccccacccgtggacgagacacccgagccaagcgccgagaaccagagcaccgagggcacacccgagcagccacccctgatcaccgaggacgagacaaggacccggaccccagagcccatcattatcgaggaagaggaagaggacagcatcagcctgctgagcgacggccccacccaccaggtgctgcaggtggaggccgacatccacggcccacccagcgtgtccagctccagctggagcatcccacacgccagcgacttcgacgtggacagcctgagcatcctggacaccctggagggcgccagcgtgacctccggcgccaccagcgccgagaccaacagctacttcgccaagagcatggagttcctggccaggcccgtgccagctcccaggaccgtgttcaggaacccaccccacccagctcccaggaccaggaccccaagcctggctcccagcagggcctgcagcaggaccagcctggtgagcaccccacccggcgtgaacagggtgatcaccagggaggaactggaggccctgacacccagcaggacccccagcaggtccgtgagcaggactagtctggtgtccaacccacccggcgtgaacagggtgatcaccagggaggaattcgaggccttcgtggcccagcaacagagacggttcgacgccggcgcctacatcttcagcagcgacaccggccagggacacctgcagcaaaagagcgtgaggcagaccgtgctgagcgaggtggtgctggagaggaccgagctggaaatcagctacgcccccaggctggaccaggagaaggaggaactgctcaggaagaaactgcagctgaaccccaccccagccaacaggagcaggtaccagagcaggaaggtggagaacatgaaggccatcaccgccaggcggatcctgcagggcctgggacactacctgaaggccgagggcaaggtggagtgctacaggaccctgcaccccgtgccactgtacagctccagcgtgaacagggccttctccagccccaaggtggccgtggaggcctgcaacgctatgctgaaggagaacttccccaccgtggccagctactgcatcatccccgagtacgacgcctacctggacatggtggacggcgccagctgctgcctggacaccgccagcttctgccccgccaagctgaggagcttccccaagaaacacagctacctggagcccaccatcaggagcgccgtgcccagcgccatccagaacaccctgcagaacgtgctggccgctgccaccaagaggaactgcaacgtgacccagatgagggagctgcccgtgctggacagcgctgccttcaacgtggagtgcttcaagaaatacgcctgcaacaacgagtactgggagaccttcaaggagaaccccatcaggctgaccgaagagaacgtggtgaactacatcaccaagctgaagggccccaaggccgctgccctgttcgctaagacccacaacctgaacatgctgcaggacatcccaatggacaggttcgtgatggacctgaagagggacgtgaaggtgacacccggcaccaagcacaccgaggagaggcccaaggtgcaggtgatccaggccgctgacccactggccaccgcctacctgtgcggcatccacagggagctggtgaggcggctgaacgccgtgctgctgcccaacatccacaccctgttcgacatgagcgccgaggacttcgacgccatcatcgccgagcacttccagcccggcgactgcgtgctggagaccgacatcgccagcttcgacaagagcgaggatgacgctatggccctgaccgctctgatgatcctggaggacctgggcgtggacgccgagctgctcaccctgatcgaggctgccttcggcgagatcagctccatccacctgcccaccaagaccaagttcaagttcggcgctatgatgaaaagcggaatgttcctgaccctgttcgtgaacaccgtgatcaacattgtgatcgccagcagggtgctgcgggagaggctgaccggcagcccctgcgctgccttcatcggcgacgacaacatcgtgaagggcgtgaaaagcgacaagctgatggccgacaggtgcgccacctggctgaacatggaggtgaagatcatcgacgccgtggtgggcgagaaggccccctacttctgcggcggattcatcctgtgcgacagcgtgaccggcaccgcctgcagggtggccgaccccctgaagaggctgttcaagctgggcaagccactggccgctgacgatgagcacgacgatgacaggcggagggccctgcacgaggaaagcaccaggtggaacagggtgggcatcctgagcgagctgtgcaaggccgtggagagcaggtacgagaccgtgggcaccagcatcatcgtgatggctatgaccacactggccagctccgtcaagagcttctcctacctgaggggggcccctataactctctacggctaacctgaatggactacgacatagtctagtccgccaaggccgccaccatgagagtgacagcccctagaaccttactgcttctgctttggggagctgttgctctgacagagacatgggctggatcttaccacagccccagctacgcctaccaccagttcgagagggggggaggaggctccgggggaggaggctccctgaagatcagccaggccgtgcacgccgcccacgccgagatcaacgaggccggccgggaggtgatcgtgggcattgtcgctggcctggccgtcctcgccgtggtggtgattggagctgtggtcgcagctgttatgtgcagaagaaagtcatccggcggaaagggaggctcctactctcaggctgcttctgctacagtgcctagagctcttatgtgtttatctcagctgggcggcggaggcagcgactacaaggacgacgatgacaagtaaactcgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaatctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 109 - protein, 2512 aa, synthetic construct
  REGION 1..2512: note = Synthetic polypeptide
MEKPVVNVDVDPQSPFVVQLQKSFPQFEVVAQQVTPNDHANARAFSHLASKLIELEVPTTATILDIGSAPARRMFSEHQYHCVCPMRSPEDPDRMMKYASKLAEKACKITNKNLHEKIKDLRTVLDTPDAETPSLCFHNDVTCNMRAEYSVMQDVYINAPGTIYHQAMKGVRTLYWIGFDTTQFMFSAMAGSYPAYNTNWADEKVLEARNIGLCSTKLSEGRTGKLSIMRKKELKPGSRVYFSVGSTLYPEHRASLQSWHLPSVFHLNGKQSYTCRCDTVVSCEGYVVKKITISPGITGETVGYAVTHNSEGFLLCKVTDTVKGERVSFPVCTYIPATICDQMTGIMATDISPDDAQKLLVGLNQRIVINGRTNRNTNTMQNYLLPIIAQGFSKWAKERKDDLDNEKMLGTRERKLTYGCLWAFRTKKVHSFYRPPGTQTCVKVPASFSAFPMSSVWTTSLPMSLRQKLKLALQPKKEEKLLQVSEELVMEAKAAFEDAQEEARAEKLREALPPLVADKGIEAAAEVVCEVEGLQADIGAALVETPRGHVRIIPQANDRMIGQYIVVSPNSVLKNAKLAPAHPLADQVKIITHSGRSGRYAVEPYDAKVLMPAGGAVPWPEFLALSESATLVYNEREFVNRKLYHIAMHGPAKNTEEEQYKVTKAELAETEYVFDVDKKRCVKKEEASGLVLSGELTNPPYHELALEGLKTRPAVPYKVETIGVIGTPGSGKSAIIKSTVTARDLVTSGKKENCREIEADVLRLRGMQITSKTVDSVMLNGCHKAVEVLYVDEAFACHAGALLALIAIVRPRKKVVLCGDPMQCGFFNMMQLKVHFNHPEKDICTKTFYKYISRRCTQPVTAIVSTLHYDGKMKTTNPCKKNIEIDITGATKPKPGDIILTCFRGWVKQLQIDYPGHEVMTAAASQGLTRKGVYAVRQKVNENPLYAITSEHVNVLLTRTEDRLVWKTLQGDPWIKQLTNIPKGNFQATIEDWEAEHKGIIAAINSPTPRANPFSCKTNVCWAKALEPILATAGIVLTGCQWSELFPQFADDKPHSAIYALDVICIKFFGMDLTSGLFSKQSIPLTYHPADSARPVAHWDNSPGTRKYGYDHAIAAELSRRFPVFQLAGKGTQLDLQTGRTRVISAQHNLVPVNRNLPHALVPEYKEKQPGPVEKFLNQFKHHSVLVVSEEKIEAPRKRIEWIAPIGIAGADKNYNLAFGFPPQARYDLVFINIGTKYRNHHFQQCEDHAATLKTLSRSALNCLNPGGTLVVKSYGYADRNSEDVVTALARKFVRVSAARPDCVSSNTEMYLIFRQLDNSRTRQFTPHHLNCVISSVYEGTRDGVGAAPSYRTKRENIADCQEEAVVNAANPLGRPGEGVCRAIYKRWPTSFTDSATETGTARMTVCLGKKVIHAVGPDFRKHPEAEALKLLQNAYHAVADLVNEHNIKSVAIPLLSTGIYAAGKDRLEVSLNCLTTALDRTDADVTIYCLDKKWKERIDAALQLKESVTELKDEDMEIDDELVWIHPDSCLKGRKGFSTTKGKLYSYFEGTKFHQAAKDMAEIKVLFPNDQESNEQLCAYILGETMEAIREKCPVDHNPSSSPPKTLPCLCMYAMTPERVHRLRSNNVKEVTVCSSTPLPKHKIKNVQKVQCTKVVLFNPHTPAFVPARKYIEVPEQPTAPPAQAEEAPEVVATPSPSTADNTSLDVTDISLDMDDSSEGSLFSSFSGSDNSITSMDSWSSGPSSLEIVDRRQVVVADVHAVQEPAPIPPPRLKKMARLAAARKEPTPPASNSSESLHLSFGGVSMSLGSIFDGETARQAAVQPLATGPTDVPMSFGSFSDGEIDELSRRVTESEPVLFGSFEPGEVNSIISSRSAVSFPLRKQRRRRRSRRTEYLTGVGGYIFSTDTGPGHLQKKSVLQNQLTEPTLERNVLERIHAPVLDTSKEEQLKLRYQMMPTEANKSRYQSRKVENQKAITTERLLSGLRLYNSATDQPECYKITYPKPLYSSSVPANYSDPQFAVAVCNNYLHENYPTVASYQITDEYDAYLDMVDGTVACLDTATFCPAKLRSYPKKHEYRAPNIRSAVPSAMQNTLQNVLIAATKRNCNVTQMRELPTLDSATFNVECFRKYACNDEYWEEFARKPIRITTEFVTAYVARLKGPKAAALFAKTYNLVPLQEVPMDRFVMDMKRDVKVTPGTKHTEERPKVQVIQAAEPLATAYLCGIHRELVRRLTAVLLPNIHTLFDMSAEDFDAIIAEHFKQGDPVLETDIASFDKSQDDAMALTGLMILEDLGVDQPLLDLIECAFGEISSTHLPTGTRFKFGAMMKSGMFLTLFVNTVLNVVIASRVLEERLKTSRCAAFIGDDNIIHGVVSDKEMAERCATWLNMEVKIIDAVIGERPPYFCGGFILQDSVTSTACRVADPLKRLFKLGKPLPADDEQDEDRRRALLDETKAWFRVGITGTLAVAVTTRYEVDNITPVLLALRTFAQSKRAFQAIRGEIKHLYGGPK

SEQ ID NO: 110 - protein, 9 aa, synthetic construct
  REGION 1..9: note = Synthetic polypeptide
SPSYVYHQF

SEQ ID NO: 111 - protein, 9 aa, synthetic construct
  REGION 1..9: note = Synthetic polypeptide
SPSYAYHQF

SEQ ID NO: 112 - protein, 9 aa, synthetic construct
  REGION 1..9: note = Synthetic polypeptide
TPHPARIGL

SEQ ID NO: 113 - RNA, 1701 nt, synthetic construct
  misc_feature 1..1701: note = Synthetic polynucleotide
  source 1..1701: mol_type = other RNA
atgaaggctatcctggtggtgctgctctacacctttgccacagccaatgctgacaccctgtgtattggctaccatgccaacaacagcacagacacagtggacacagtgttggagaagaatgtgacagtgacccactctgtgaacctgttggaggacaaacacaatggcaaactgtgtaaactgaggggagtggctccactgcacctgggcaagtgtaacattgctggctggattctgggcaaccctgagtgtgagtccctgagcacagcctcctcctggtcctacattgtggagacaccatcctctgacaatggcacttgttaccctggagacttcattgactatgaggaactgagggaacaactttcctctgtgtcctcctttgagaggtttgagatttttccaaagacctcctcctggccaaaccatgacagcaacaagggagtgacagcagcctgtccacatgctggagccaagtccttctacaagaacctgatttggctggtgaagaagggcaactcctacccaaaactgagcaagtcctacatcaatgacaagggcaaggaggtgctggtgctgtggggcatccaccacccaagcacctctgctgaccaacagtccctctaccagaatgctgacgcctatgtgtttgtgggctccagcagatacagcaagaagttcaagcctgagattgccatcagaccaaaggtgagggatcaggagggcaggatgaactactactggaccctggtggaacctggagacaagattacctttgaggctacaggcaacctggtggtgccaagatatgcctttgctatggagaggaatgctggctctggcatcatcatctctgacacacctgtccatgactgtaacaccacttgtcagacaccaaagggagccatcaacacctccctgccattccagaacatccacccaatcaccattggcaagtgtccaaaatatgtcaagagcaccaaactgagactggctacaggactgaggaacatcccaagcatccagagcaggggactgtttggagccattgctggcttcattgagggaggctggacagggatggtggatggctggtatggctaccaccaccagaatgaacagggctctggctatgctgctgacctgaaaagcacccagaatgccattgatgagattaccaacaaggtgaactctgtgattgagaagatgaacacccagttcacagcagtgggcaaggagttcaaccacttggagaagaggattgagaacctgaacaagaaggtggatgatggcttcctggacatctggacctacaatgctgaactgctggtgctgttggagaatgagaggaccctggactaccatgacagcaatgtgaagaacctctatgagaaggtgaggagccaacttaaaaacaatgccaaggagattggcaatggctgttttgagttctaccacaagtgtgacaacacttgtatggagtctgtgaagaatggcacctatgactacccaaaatactctgaggaggctaaactgaacagggaggagattgatggagtgaaattggagagcaccaggatttaccagatcctggccatctacagcaccgtggccagcagcctggtgctggtggtgagcctgggcgccatcagcttctggatgtgcagcaacggcagcttgcagtgcaggatctgcatctaa

SEQ ID NO: 114 - protein, 566 aa, synthetic construct
  REGION 1..566: note = Synthetic polypeptide
MKAILVVLLYTFATANADTLCIGYHANNSTDTVDTVLEKNVTVTHSVNLLEDKHNGKLCKLRGVAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGSSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNIPSIQSRGLFGAIAGFIEGGWTGMVDGWYGYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGVKLESTRIYQILAIYSTVASSLVLVVSLGAISFWMCSNGSLQCRICI

SEQ ID NO: 115 - RNA, 9911 nt, synthetic construct
  misc_feature 1..9911: note = Synthetic polynucleotide
  source 1..9911: mol_type = other RNA
gataggcggcgcatgagagaagcccagaccaattacctacccaaataggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaatagtcagcatagtacatttcatctgactaatactacaacaccaccaccatgaatagaggattctttaacatgctcggccgccgccccttcccggcccccactgccatgtggaggccgcggagaaggaggcaggcggccccgggaagcggagctactaacttcagcctgctgaagcaggctggagacgtggaggagaaccctggacctgagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctctgaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgctcactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatatcgcaccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgacagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagtccctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaaggcgcgccgtttaaacggccggccttaattaagtaacgatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaatctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 116 - RNA, 2117 nt, synthetic construct
  misc_feature 1..2117: note = Synthetic polynucleotide
  source 1..2117: mol_type = other RNA
aggaaacttaagtcaacacaacatatacaaaacaaacgaatctcaagcaatcaagcattctacttctattgcagcaatttaaatcatttcttttaaagcaaaagcaattttctgaaaattttcaccatttacgaacgatagccaccatgaaggctatcctggtggtgctgctctacacctttgccacagccaatgctgacaccctgtgtattggctaccatgccaacaacagcacagacacagtggacacagtgttggagaagaatgtgacagtgacccactctgtgaacctgttggaggacaaacacaatggcaaactgtgtaaactgaggggagtggctccactgcacctgggcaagtgtaacattgctggctggattctgggcaaccctgagtgtgagtccctgagcacagcctcctcctggtcctacattgtggagacaccatcctctgacaatggcacttgttaccctggagacttcattgactatgaggaactgagggaacaactttcctctgtgtcctcctttgagaggtttgagatttttccaaagacctcctcctggccaaaccatgacagcaacaagggagtgacagcagcctgtccacatgctggagccaagtccttctacaagaacctgatttggctggtgaagaagggcaactcctacccaaaactgagcaagtcctacatcaatgacaagggcaaggaggtgctggtgctgtggggcatccaccacccaagcacctctgctgaccaacagtccctctaccagaatgctgacgcctatgtgtttgtgggctccagcagatacagcaagaagttcaagcctgagattgccatcagaccaaaggtgagggatcaggagggcaggatgaactactactggaccctggtggaacctggagacaagattacctttgaggctacaggcaacctggtggtgccaagatatgcctttgctatggagaggaatgctggctctggcatcatcatctctgacacacctgtccatgactgtaacaccacttgtcagacaccaaagggagccatcaacacctccctgccattccagaacatccacccaatcaccattggcaagtgtccaaaatatgtcaagagcaccaaactgagactggctacaggactgaggaacatcccaagcatccagagcaggggactgtttggagccattgctggcttcattgagggaggctggacagggatggtggatggctggtatggctaccaccaccagaatgaacagggctctggctatgctgctgacctgaaaagcacccagaatgccattgatgagattaccaacaaggtgaactctgtgattgagaagatgaacacccagttcacagcagtgggcaaggagttcaaccacttggagaagaggattgagaacctgaacaagaaggtggatgatggcttcctggacatctggacctacaatgctgaactgctggtgctgttggagaatgagaggaccctggactaccatgacagcaatgtgaagaacctctatgagaaggtgaggagccaacttaaaaacaatgccaaggagattggcaatggctgttttgagttctaccacaagtgtgacaacacttgtatggagtctgtgaagaatggcacctatgactacccaaaatactctgaggaggctaaactgaacagggaggagattgatggagtgaaattggagagcaccaggatttaccagatcctggccatctacagcaccgtggccagcagcctggtgctggtggtgagcctgggcgccatcagcttctggatgtgcagcaacggcagcttgcagtgcaggatctgcatctaaactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 117 - DNA, 10508 bp, synthetic construct
  misc_feature 1..10508: note = Synthetic polynucleotide
  source 1..10508: mol_type = other DNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgccgtcgacggccccaccagcctgtaccaccaggccaacaagggcgtgagggtggcctactggatcggcttcgacaccacacccttcatgttcaagaacctggccggcgcctaccccagctacagcaccaactgggccgacgagaccgtgctgaccgccaggaacatcggcctgtgcagcagcgacgtgatggagaggagccggagaggcatgagcatcctgaggaagaaatacctgaagcccagcaacaacgtgctgttcagcgtgggcagcaccatctaccacgagaagagggacctgctcaggagctggcacctgcccagcgtgttccacctgaggggcaagcagaactacacctgcaggtgcgagaccatcgtgagctgcgacggctacgtggtgaagaggatcgccatcagccccggcctgtacggcaagcccagcggctacgccgctacaatgcacagggagggcttcctgtgctgcaaggtgaccgacaccctgaacggcgagagggtgagcttccccgtgtgcacctacgtgcccgccaccctgtgcgaccagatgaccggcatcctggccaccgacgtgagcgccgacgacgcccagaagctgctcgtgggcctgaaccagaggatcgtggtcaacggcaggacccagaggaacaccaacacaatgaagaactacctgctgcccgtggtggcccaggctttcgccaggtgggccaaggagtacaaggaggaccaggaagacgagaggcccctgggcctgagggacaggcagctggtgatgggctgctgctgggccttcaggcggcacaagatcaccagcatctacaagaggcccgacacccagaccatcatcaaggtgaacagcgacttccacagcttcgtgctgcccaggatcggcagcaacaccctggagatcggcctgaggacccggatcaggaagatgctggaggaacacaaggagcccagcccactgatcaccgccgaggacgtgcaggaggccaagtgcgctgccgacgaggccaaggaggtgagggaggccgaggaactgagggccgccctgccacccctggctgccgacgtggaggaacccaccctggaagccgacgtggacctgatgctgcaggaggccggcgccggaagcgtggagacacccaggggcctgatcaaggtgaccagctacgacggcgaggacaagatcggcagctacgccgtgctgagcccacaggccgtgctgaagtccgagaagctgagctgcatccacccactggccgagcaggtgatcgtgatcacccacagcggcaggaagggcaggtacgccgtggagccctaccacggcaaggtggtcgtgcccgagggccacgccatccccgtgcaggacttccaggccctgagcgagagcgccaccatcgtgtacaacgagagggagttcgtgaacaggtacctgcaccatatcgccacccacggcggagccctgaacaccgacgaggaatactacaagaccgtgaagcccagcgagcacgacggcgagtacctgtacgacatcgacaggaagcagtgcgtgaagaaagagctggtgaccggcctgggactgaccggcgagctggtggacccacccttccacgagttcgcctacgagagcctgaggaccagacccgccgctccctaccaggtgcccaccatcggcgtgtacggcgtgcccggcagcggaaagagcggcatcatcaagagcgccgtgaccaagaaagacctggtggtcagcgccaagaaagagaactgcgccgagatcatcagggacgtgaagaagatgaaaggcctggacgtgaacgcgcgcaccgtggacagcgtgctgctgaacggctgcaagcaccccgtggagaccctgtacatcgacgaggccttcgcttgccacgccggcaccctgagggccctgatcgccatcatcaggcccaagaaagccgtgctgtgcggcgaccccaagcagtgcggcttcttcaacatgatgtgcctgaaggtgcacttcaaccacgagatctgcacccaggtgttccacaagagcatcagcaggcggtgcaccaagagcgtgaccagcgtcgtgagcaccctgttctacgacaagaaaatgaggaccaccaaccccaaggagaccaaaatcgtgatcgacaccacaggcagcaccaagcccaagcaggacgacctgatcctgacctgcttcaggggctgggtgaagcagctgcagatcgactacaagggcaacgagatcatgaccgccgctgccagccagggcctgaccaggaagggcgtgtacgccgtgaggtacaaggtgaacgagaacccactgtacgctcccaccagcgagcacgtgaacgtgctgctgaccaggaccgaggacaggatcgtgtggaagaccctggccggcgacccctggatcaagaccctgaccgccaagtaccccggcaacttcaccgccaccatcgaagagtggcaggccgagcacgacgccatcatgaggcacatcctggagaggcccgaccccaccgacgtgttccagaacaaggccaacgtgtgctgggccaaggccctggtgcccgtgctgaagaccgccggcatcgacatgaccacagagcagtggaacaccgtggactacttcgagaccgacaaggcccacagcgccgagatcgtgctgaaccagctgtgcgtgaggttcttcggcctggacctggacagcggcctgttcagcgcccccaccgtgccactgagcatcaggaacaaccactgggacaacagccccagcccaaacatgtacggcctgaacaaggaggtggtcaggcagctgagcaggcggtacccacagctgcccagggccgtggccaccggcagggtgtacgacatgaacaccggcaccctgaggaactacgaccccaggatcaacctggtgcccgtgaacaggcggctgccccacgccctggtgctgcaccacaacgagcacccacagagcgacttcagctccttcgtgagcaagctgaaaggcaggaccgtgctggtcgtgggcgagaagctgagcgtgcccggcaagatggtggactggctgagcgacaggcccgaggccaccttccgggccaggctggacctcggcatccccggcgacgtgcccaagtacgacatcatcttcgtgaacgtcaggaccccatacaagtaccaccattaccagcagtgcgaggaccacgccatcaagctgagcatgctgaccaagaaggcctgcctgcacctgaaccccggaggcacctgcgtgagcatcggctacggctacgccgacagggccagcgagagcatcattggcgccatcgccaggctgttcaagttcagcagggtgtgcaaacccaagagcagcctggaggaaaccgaggtgctgttcgtgttcatcggctacgaccggaaggccaggacccacaacccctacaagctgagcagcaccctgacaaacatctacaccggcagcaggctgcacgaggccggctgcgcccccagctaccacgtggtcaggggcgatatcgccaccgccaccgagggcgtgatcatcaacgctgccaacagcaagggccagcccggaggcggagtgtgcggcgccctgtacaagaagttccccgagagcttcgacctgcagcccatcgaggtgggcaaggccaggctggtgaagggcgccgctaagcacatcatccacgccgtgggccccaacttcaacaaggtgagcgaggtggaaggcgacaagcagctggccgaagcctacgagagcatcgccaagatcgtgaacgacaataactacaagagcgtggccatcccactgctcagcaccggcatcttcagcggcaacaaggacaggctgacccagagcctgaaccacctgctcaccgccctggacaccaccgatgccgacgtggccatctactgcagggacaagaagtgggagatgaccctgaaggaggccgtggccaggcgggaggccgtggaagagatctgcatcagcgacgactccagcgtgaccgagcccgacgccgagctggtgagggtgcaccccaagagctccctggccggcaggaagggctacagcaccagcgacggcaagaccttcagctacctggagggcaccaagttccaccaggccgctaaggacatcgccgagatcaacgctatgtggcccgtggccaccgaggccaacgagcaggtgtgcatgtacatcctgggcgagagcatgtccagcatcaggagcaagtgccccgtggaggaaagcgaggccagcacaccacccagcaccctgccctgcctgtgcatccacgctatgacacccgagagggtgcagcggctgaaggccagcaggcccgagcagatcaccgtgtgcagctccttcccactgcccaagtacaggatcaccggcgtgcagaagatccagtgcagccagcccatcctgttcagcccaaaggtgcccgcctacatccaccccaggaagtacctggtggagaccccacccgtggacgagacacccgagccaagcgccgagaaccagagcaccgagggcacacccgagcagccacccctgatcaccgaggacgagacaaggacccggaccccagagcccatcattatcgaggaagaggaagaggacagcatcagcctgctgagcgacggccccacccaccaggtgctgcaggtggaggccgacatccacggcccacccagcgtgtccagctccagctggagcatcccacacgccagcgacttcgacgtggacagcctgagcatcctggacaccctggagggcgccagcgtgacctccggcgccaccagcgccgagaccaacagctacttcgccaagagcatggagttcctggccaggcccgtgccagctcccaggaccgtgttcaggaacccaccccacccagctcccaggaccaggaccccaagcctggctcccagcagggcctgcagcaggaccagcctggtgagcaccccacccggcgtgaacagggtgatcaccagggaggaactggaggccctgacacccagcaggacccccagcaggtccgtgagcaggactagtctggtgtccaacccacccggcgtgaacagggtgatcaccagggaggaattcgaggccttcgtggcccagcaacagagacggttcgacgccggcgcctacatcttcagcagcgacaccggccagggacacctgcagcaaaagagcgtgaggcagaccgtgctgagcgaggtggtgctggagaggaccgagctggaaatcagctacgcccccaggctggaccaggagaaggaggaactgctcaggaagaaactgcagctgaaccccaccccagccaacaggagcaggtaccagagcaggaaggtggagaacatgaaggccatcaccgccaggcggatcctgcagggcctgggacactacctgaaggccgagggcaaggtggagtgctacaggaccctgcaccccgtgccactgtacagctccagcgtgaacagggccttctccagccccaaggtggccgtggaggcctgcaacgctatgctgaaggagaacttccccaccgtggccagctactgcatcatccccgagtacgacgcctacctggacatggtggacggcgccagctgctgcctggacaccgccagcttctgccccgccaagctgaggagcttccccaagaaacacagctacctggagcccaccatcaggagcgccgtgcccagcgccatccagaacaccctgcagaacgtgctggccgctgccaccaagaggaactgcaacgtgacccagatgagggagctgcccgtgctggacagcgctgccttcaacgtggagtgcttcaagaaatacgcctgcaacaacgagtactgggagaccttcaaggagaaccccatcaggctgaccgaagagaacgtggtgaactacatcaccaagctgaagggccccaaggccgctgccctgttcgctaagacccacaacctgaacatgctgcaggacatcccaatggacaggttcgtgatggacctgaagagggacgtgaaggtgacacccggcaccaagcacaccgaggagaggcccaaggtgcaggtgatccaggccgctgacccactggccaccgcctacctgtgcggcatccacagggagctggtgaggcggctgaacgccgtgctgctgcccaacatccacaccctgttcgacatgagcgccgaggacttcgacgccatcatcgccgagcacttccagcccggcgactgcgtgctggagaccgacatcgccagcttcgacaagagcgaggatgacgctatggccctgaccgctctgatgatcctggaggacctgggcgtggacgccgagctgctcaccctgatcgaggctgccttcggcgagatcagctccatccacctgcccaccaagaccaagttcaagttcggcgctatgatgaaaagcggaatgttcctgaccctgttcgtgaacaccgtgatcaacattgtgatcgccagcagggtgctgcgggagaggctgaccggcagcccctgcgctgccttcatcggcgacgacaacatcgtgaagggcgtgaaaagcgacaagctgatggccgacaggtgcgccacctggctgaacatggaggtgaagatcatcgacgccgtggtgggcgagaaggccccctacttctgcggcggattcatcctgtgcgacagcgtgaccggcaccgcctgcagggtggccgaccccctgaagaggctgttcaagctgggcaagccactggccgctgacgatgagcacgacgatgacaggcggagggccctgcacgaggaaagcaccaggtggaacagggtgggcatcctgagcgagctgtgcaaggccgtggagagcaggtacgagaccgtgggcaccagcatcatcgtgatggctatgaccacactggccagctccgtcaagagcttctcctacctgaggggggcccctataactctctacggctaacctgaatggactacgacatagtctagccaccatgagcaagatctacatcgacgagcggagcaacgccgagatcgtgtgcgaggccatcaagaccatcggcatcgagggcgccaccgccgcccagctgaccaggcagctgaacatggagaagcgggaggtgaacaaggccctgtacgacctgcagaggagcgctatggtgtactccagcgacgacatccctccccggtggttcatgaccaccgaggccgacaagcccgacgccgacgctatggccgacgtgatcatcgacgacgtgagcagggagaagtccatgagggaggaccacaagagcttcgacgacgtgatccccgccaagaagatcatcgactggaagggcgccaaccccgtgaccgtgatcaacgagtactgccagatcaccaggagggactggagcttccggatcgagagcgtgggccccagcaacagccccaccttctacgcctgcgtggacatcgacggcagggtgttcgacaaggccgacggcaagagcaagcgggacgccaagaacaacgccgccaagctggccgtggacaagctgctgggctacgtgatcatccggttctaaactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctagagctccgtcaagagcttctcctacctgaggggggcccctataactctctacggctaacctgaatggactacgacatagtctagccaccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgacagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagtccctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaactcgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaatctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 118 - RNA, 11075 nt, synthetic construct
  misc_feature 1..11075: note = Synthetic polynucleotide
  source 1..11075: mol_type = other RNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgccgtcgacggccccaccagcctgtaccaccaggccaacaagggcgtgagggtggcctactggatcggcttcgacaccacacccttcatgttcaagaacctggccggcgcctaccccagctacagcaccaactgggccgacgagaccgtgctgaccgccaggaacatcggcctgtgcagcagcgacgtgatggagaggagccggagaggcatgagcatcctgaggaagaaatacctgaagcccagcaacaacgtgctgttcagcgtgggcagcaccatctaccacgagaagagggacctgctcaggagctggcacctgcccagcgtgttccacctgaggggcaagcagaactacacctgcaggtgcgagaccatcgtgagctgcgacggctacgtggtgaagaggatcgccatcagccccggcctgtacggcaagcccagcggctacgccgctacaatgcacagggagggcttcctgtgctgcaaggtgaccgacaccctgaacggcgagagggtgagcttccccgtgtgcacctacgtgcccgccaccctgtgcgaccagatgaccggcatcctggccaccgacgtgagcgccgacgacgcccagaagctgctcgtgggcctgaaccagaggatcgtggtcaacggcaggacccagaggaacaccaacacaatgaagaactacctgctgcccgtggtggcccaggctttcgccaggtgggccaaggagtacaaggaggaccaggaagacgagaggcccctgggcctgagggacaggcagctggtgatgggctgctgctgggccttcaggcggcacaagatcaccagcatctacaagaggcccgacacccagaccatcatcaaggtgaacagcgacttccacagcttcgtgctgcccaggatcggcagcaacaccctggagatcggcctgaggacccggatcaggaagatgctggaggaacacaaggagcccagcccactgatcaccgccgaggacgtgcaggaggccaagtgcgctgccgacgaggccaaggaggtgagggaggccgaggaactgagggccgccctgccacccctggctgccgacgtggaggaacccaccctggaagccgacgtggacctgatgctgcaggaggccggcgccggaagcgtggagacacccaggggcctgatcaaggtgaccagctacgacggcgaggacaagatcggcagctacgccgtgctgagcccacaggccgtgctgaagtccgagaagctgagctgcatccacccactggccgagcaggtgatcgtgatcacccacagcggcaggaagggcaggtacgccgtggagccctaccacggcaaggtggtcgtgcccgagggccacgccatccccgtgcaggacttccaggccctgagcgagagcgccaccatcgtgtacaacgagagggagttcgtgaacaggtacctgcaccatatcgccacccacggcggagccctgaacaccgacgaggaatactacaagaccgtgaagcccagcgagcacgacggcgagtacctgtacgacatcgacaggaagcagtgcgtgaagaaagagctggtgaccggcctgggactgaccggcgagctggtggacccacccttccacgagttcgcctacgagagcctgaggaccagacccgccgctccctaccaggtgcccaccatcggcgtgtacggcgtgcccggcagcggaaagagcggcatcatcaagagcgccgtgaccaagaaagacctggtggtcagcgccaagaaagagaactgcgccgagatcatcagggacgtgaagaagatgaaaggcctggacgtgaacgcgcgcaccgtggacagcgtgctgctgaacggctgcaagcaccccgtggagaccctgtacatcgacgaggccttcgcttgccacgccggcaccctgagggccctgatcgccatcatcaggcccaagaaagccgtgctgtgcggcgaccccaagcagtgcggcttcttcaacatgatgtgcctgaaggtgcacttcaaccacgagatctgcacccaggtgttccacaagagcatcagcaggcggtgcaccaagagcgtgaccagcgtcgtgagcaccctgttctacgacaagaaaatgaggaccaccaaccccaaggagaccaaaatcgtgatcgacaccacaggcagcaccaagcccaagcaggacgacctgatcctgacctgcttcaggggctgggtgaagcagctgcagatcgactacaagggcaacgagatcatgaccgccgctgccagccagggcctgaccaggaagggcgtgtacgccgtgaggtacaaggtgaacgagaacccactgtacgctcccaccagcgagcacgtgaacgtgctgctgaccaggaccgaggacaggatcgtgtggaagaccctggccggcgacccctggatcaagaccctgaccgccaagtaccccggcaacttcaccgccaccatcgaagagtggcaggccgagcacgacgccatcatgaggcacatcctggagaggcccgaccccaccgacgtgttccagaacaaggccaacgtgtgctgggccaaggccctggtgcccgtgctgaagaccgccggcatcgacatgaccacagagcagtggaacaccgtggactacttcgagaccgacaaggcccacagcgccgagatcgtgctgaaccagctgtgcgtgaggttcttcggcctggacctggacagcggcctgttcagcgcccccaccgtgccactgagcatcaggaacaaccactgggacaacagccccagcccaaacatgtacggcctgaacaaggaggtggtcaggcagctgagcaggcggtacccacagctgcccagggccgtggccaccggcagggtgtacgacatgaacaccggcaccctgaggaactacgaccccaggatcaacctggtgcccgtgaacaggcggctgccccacgccctggtgctgcaccacaacgagcacccacagagcgacttcagctccttcgtgagcaagctgaaaggcaggaccgtgctggtcgtgggcgagaagctgagcgtgcccggcaagatggtggactggctgagcgacaggcccgaggccaccttccgggccaggctggacctcggcatccccggcgacgtgcccaagtacgacatcatcttcgtgaacgtcaggaccccatacaagtaccaccattaccagcagtgcgaggaccacgccatcaagctgagcatgctgaccaagaaggcctgcctgcacctgaaccccggaggcacctgcgtgagcatcggctacggctacgccgacagggccagcgagagcatcattggcgccatcgccaggctgttcaagttcagcagggtgtgcaaacccaagagcagcctggaggaaaccgaggtgctgttcgtgttcatcggctacgaccggaaggccaggacccacaacccctacaagctgagcagcaccctgacaaacatctacaccggcagcaggctgcacgaggccggctgcgcccccagctaccacgtggtcaggggcgatatcgccaccgccaccgagggcgtgatcatcaacgctgccaacagcaagggccagcccggaggcggagtgtgcggcgccctgtacaagaagttccccgagagcttcgacctgcagcccatcgaggtgggcaaggccaggctggtgaagggcgccgctaagcacatcatccacgccgtgggccccaacttcaacaaggtgagcgaggtggaaggcgacaagcagctggccgaagcctacgagagcatcgccaagatcgtgaacgacaataactacaagagcgtggccatcccactgctcagcaccggcatcttcagcggcaacaaggacaggctgacccagagcctgaaccacctgctcaccgccctggacaccaccgatgccgacgtggccatctactgcagggacaagaagtgggagatgaccctgaaggaggccgtggccaggcgggaggccgtggaagagatctgcatcagcgacgactccagcgtgaccgagcccgacgccgagctggtgagggtgcaccccaagagctccctggccggcaggaagggctacagcaccagcgacggcaagaccttcagctacctggagggcaccaagttccaccaggccgctaaggacatcgccgagatcaacgctatgtggcccgtggccaccgaggccaacgagcaggtgtgcatgtacatcctgggcgagagcatgtccagcatcaggagcaagtgccccgtggaggaaagcgaggccagcacaccacccagcaccctgccctgcctgtgcatccacgctatgacacccgagagggtgcagcggctgaaggccagcaggcccgagcagatcaccgtgtgcagctccttcccactgcccaagtacaggatcaccggcgtgcagaagatccagtgcagccagcccatcctgttcagcccaaaggtgcccgcctacatccaccccaggaagtacctggtggagaccccacccgtggacgagacacccgagccaagcgccgagaaccagagcaccgagggcacacccgagcagccacccctgatcaccgaggacgagacaaggacccggaccccagagcccatcattatcgaggaagaggaagaggacagcatcagcctgctgagcgacggccccacccaccaggtgctgcaggtggaggccgacatccacggcccacccagcgtgtccagctccagctggagcatcccacacgccagcgacttcgacgtggacagcctgagcatcctggacaccctggagggcgccagcgtgacctccggcgccaccagcgccgagaccaacagctacttcgccaagagcatggagttcctggccaggcccgtgccagctcccaggaccgtgttcaggaacccaccccacccagctcccaggaccaggaccccaagcctggctcccagcagggcctgcagcaggaccagcctggtgagcaccccacccggcgtgaacagggtgatcaccagggaggaactggaggccctgacacccagcaggacccccagcaggtccgtgagcaggactagtctggtgtccaacccacccggcgtgaacagggtgatcaccagggaggaattcgaggccttcgtggcccagcaacagagacggttcgacgccggcgcctacatcttcagcagcgacaccggccagggacacctgcagcaaaagagcgtgaggcagaccgtgctgagcgaggtggtgctggagaggaccgagctggaaatcagctacgcccccaggctggaccaggagaaggaggaactgctcaggaagaaactgcagctgaaccccaccccagccaacaggagcaggtaccagagcaggaaggtggagaacatgaaggccatcaccgccaggcggatcctgcagggcctgggacactacctgaaggccgagggcaaggtggagtgctacaggaccctgcaccccgtgccactgtacagctccagcgtgaacagggccttctccagccccaaggtggccgtggaggcctgcaacgctatgctgaaggagaacttccccaccgtggccagctactgcatcatccccgagtacgacgcctacctggacatggtggacggcgccagctgctgcctggacaccgccagcttctgccccgccaagctgaggagcttccccaagaaacacagctacctggagcccaccatcaggagcgccgtgcccagcgccatccagaacaccctgcagaacgtgctggccgctgccaccaagaggaactgcaacgtgacccagatgagggagctgcccgtgctggacagcgctgccttcaacgtggagtgcttcaagaaatacgcctgcaacaacgagtactgggagaccttcaaggagaaccccatcaggctgaccgaagagaacgtggtgaactacatcaccaagctgaagggccccaaggccgctgccctgttcgctaagacccacaacctgaacatgctgcaggacatcccaatggacaggttcgtgatggacctgaagagggacgtgaaggtgacacccggcaccaagcacaccgaggagaggcccaaggtgcaggtgatccaggccgctgacccactggccaccgcctacctgtgcggcatccacagggagctggtgaggcggctgaacgccgtgctgctgcccaacatccacaccctgttcgacatgagcgccgaggacttcgacgccatcatcgccgagcacttccagcccggcgactgcgtgctggagaccgacatcgccagcttcgacaagagcgaggatgacgctatggccctgaccgctctgatgatcctggaggacctgggcgtggacgccgagctgctcaccctgatcgaggctgccttcggcgagatcagctccatccacctgcccaccaagaccaagttcaagttcggcgctatgatgaaaagcggaatgttcctgaccctgttcgtgaacaccgtgatcaacattgtgatcgccagcagggtgctgcgggagaggctgaccggcagcccctgcgctgccttcatcggcgacgacaacatcgtgaagggcgtgaaaagcgacaagctgatggccgacaggtgcgccacctggctgaacatggaggtgaagatcatcgacgccgtggtgggcgagaaggccccctacttctgcggcggattcatcctgtgcgacagcgtgaccggcaccgcctgcagggtggccgaccccctgaagaggctgttcaagctgggcaagccactggccgctgacgatgagcacgacgatgacaggcggagggccctgcacgaggaaagcaccaggtggaacagggtgggcatcctgagcgagctgtgcaaggccgtggagagcaggtacgagaccgtgggcaccagcatcatcgtgatggctatgaccacactggccagctccgtcaagagcttctcctacctgaggggggcccctataactctctacggctaacctgaatggactacgacatagtctagtccgccaaggccgccaccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgacagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagtccctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaactcgagccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatgagcaagatctacatcgacgagcggagcaacgccgagatcgtgtgcgaggccatcaagaccatcggcatcgagggcgccaccgccgcccagctgaccaggcagctgaacatggagaagcgggaggtgaacaaggccctgtacgacctgcagaggagcgctatggtgtactccagcgacgacatccctccccggtggttcatgaccaccgaggccgacaagcccgacgccgacgctatggccgacgtgatcatcgacgacgtgagcagggagaagtccatgagggaggaccacaagagcttcgacgacgtgatccccgccaagaagatcatcgactggaagggcgccaaccccgtgaccgtgatcaacgagtactgccagatcaccaggagggactggagcttccggatcgagagcgtgggccccagcaacagccccaccttctacgcctgcgtggacatcgacggcagggtgttcgacaaggccgacggcaagagcaagcgggacgccaagaacaacgccgccaagctggccgtggacaagctgctgggctacgtgatcatccggttctaaacgtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaatctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 119 - RNA, 10851 nt, synthetic construct
  misc_feature 1..10851: note = Synthetic polynucleotide
  source 1..10851: mol_type = other RNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgccgtcgacggccccaccagcctgtaccaccaggccaacaagggcgtgagggtggcctactggatcggcttcgacaccacacccttcatgttcaagaacctggccggcgcctaccccagctacagcaccaactgggccgacgagaccgtgctgaccgccaggaacatcggcctgtgcagcagcgacgtgatggagaggagccggagaggcatgagcatcctgaggaagaaatacctgaagcccagcaacaacgtgctgttcagcgtgggcagcaccatctaccacgagaagagggacctgctcaggagctggcacctgcccagcgtgttccacctgaggggcaagcagaactacacctgcaggtgcgagaccatcgtgagctgcgacggctacgtggtgaagaggatcgccatcagccccggcctgtacggcaagcccagcggctacgccgctacaatgcacagggagggcttcctgtgctgcaaggtgaccgacaccctgaacggcgagagggtgagcttccccgtgtgcacctacgtgcccgccaccctgtgcgaccagatgaccggcatcctggccaccgacgtgagcgccgacgacgcccagaagctgctcgtgggcctgaaccagaggatcgtggtcaacggcaggacccagaggaacaccaacacaatgaagaactacctgctgcccgtggtggcccaggctttcgccaggtgggccaaggagtacaaggaggaccaggaagacgagaggcccctgggcctgagggacaggcagctggtgatgggctgctgctgggccttcaggcggcacaagatcaccagcatctacaagaggcccgacacccagaccatcatcaaggtgaacagcgacttccacagcttcgtgctgcccaggatcggcagcaacaccctggagatcggcctgaggacccggatcaggaagatgctggaggaacacaaggagcccagcccactgatcaccgccgaggacgtgcaggaggccaagtgcgctgccgacgaggccaaggaggtgagggaggccgaggaactgagggccgccctgccacccctggctgccgacgtggaggaacccaccctggaagccgacgtggacctgatgctgcaggaggccggcgccggaagcgtggagacacccaggggcctgatcaaggtgaccagctacgacggcgaggacaagatcggcagctacgccgtgctgagcccacaggccgtgctgaagtccgagaagctgagctgcatccacccactggccgagcaggtgatcgtgatcacccacagcggcaggaagggcaggtacgccgtggagccctaccacggcaaggtggtcgtgcccgagggccacgccatccccgtgcaggacttccaggccctgagcgagagcgccaccatcgtgtacaacgagagggagttcgtgaacaggtacctgcaccatatcgccacccacggcggagccctgaacaccgacgaggaatactacaagaccgtgaagcccagcgagcacgacggcgagtacctgtacgacatcgacaggaagcagtgcgtgaagaaagagctggtgaccggcctgggactgaccggcgagctggtggacccacccttccacgagttcgcctacgagagcctgaggaccagacccgccgctccctaccaggtgcccaccatcggcgtgtacggcgtgcccggcagcggaaagagcggcatcatcaagagcgccgtgaccaagaaagacctggtggtcagcgccaagaaagagaactgcgccgagatcatcagggacgtgaagaagatgaaaggcctggacgtgaacgcgcgcaccgtggacagcgtgctgctgaacggctgcaagcaccccgtggagaccctgtacatcgacgaggccttcgcttgccacgccggcaccctgagggccctgatcgccatcatcaggcccaagaaagccgtgctgtgcggcgaccccaagcagtgcggcttcttcaacatgatgtgcctgaaggtgcacttcaaccacgagatctgcacccaggtgttccacaagagcatcagcaggcggtgcaccaagagcgtgaccagcgtcgtgagcaccctgttctacgacaagaaaatgaggaccaccaaccccaaggagaccaaaatcgtgatcgacaccacaggcagcaccaagcccaagcaggacgacctgatcctgacctgcttcaggggctgggtgaagcagctgcagatcgactacaagggcaacgagatcatgaccgccgctgccagccagggcctgaccaggaagggcgtgtacgccgtgaggtacaaggtgaacgagaacccactgtacgctcccaccagcgagcacgtgaacgtgctgctgaccaggaccgaggacaggatcgtgtggaagaccctggccggcgacccctggatcaagaccctgaccgccaagtaccccggcaacttcaccgccaccatcgaagagtggcaggccgagcacgacgccatcatgaggcacatcctggagaggcccgaccccaccgacgtgttccagaacaaggccaacgtgtgctgggccaaggccctggtgcccgtgctgaagaccgccggcatcgacatgaccacagagcagtggaacaccgtggactacttcgagaccgacaaggcccacagcgccgagatcgtgctgaaccagctgtgcgtgaggttcttcggcctggacctggacagcggcctgttcagcgcccccaccgtgccactgagcatcaggaacaaccactgggacaacagccccagcccaaacatgtacggcctgaacaaggaggtggtcaggcagctgagcaggcggtacccacagctgcccagggccgtggccaccggcagggtgtacgacatgaacaccggcaccctgaggaactacgaccccaggatcaacctggtgcccgtgaacaggcggctgccccacgccctggtgctgcaccacaacgagcacccacagagcgacttcagctccttcgtgagcaagctgaaaggcaggaccgtgctggtcgtgggcgagaagctgagcgtgcccggcaagatggtggactggctgagcgacaggcccgaggccaccttccgggccaggctggacctcggcatccccggcgacgtgcccaagtacgacatcatcttcgtgaacgtcaggaccccatacaagtaccaccattaccagcagtgcgaggaccacgccatcaagctgagcatgctgaccaagaaggcctgcctgcacctgaaccccggaggcacctgcgtgagcatcggctacggctacgccgacagggccagcgagagcatcattggcgccatcgccaggctgttcaagttcagcagggtgtgcaaacccaagagcagcctggaggaaaccgaggtgctgttcgtgttcatcggctacgaccggaaggccaggacccacaacccctacaagctgagcagcaccctgacaaacatctacaccggcagcaggctgcacgaggccggctgcgcccccagctaccacgtggtcaggggcgatatcgccaccgccaccgagggcgtgatcatcaacgctgccaacagcaagggccagcccggaggcggagtgtgcggcgccctgtacaagaagttccccgagagcttcgacctgcagcccatcgaggtgggcaaggccaggctggtgaagggcgccgctaagcacatcatccacgccgtgggccccaacttcaacaaggtgagcgaggtggaaggcgacaagcagctggccgaagcctacgagagcatcgccaagatcgtgaacgacaataactacaagagcgtggccatcccactgctcagcaccggcatcttcagcggcaacaaggacaggctgacccagagcctgaaccacctgctcaccgccctggacaccaccgatgccgacgtggccatctactgcagggacaagaagtgggagatgaccctgaaggaggccgtggccaggcgggaggccgtggaagagatctgcatcagcgacgactccagcgtgaccgagcccgacgccgagctggtgagggtgcaccccaagagctccctggccggcaggaagggctacagcaccagcgacggcaagaccttcagctacctggagggcaccaagttccaccaggccgctaaggacatcgccgagatcaacgctatgtggcccgtggccaccgaggccaacgagcaggtgtgcatgtacatcctgggcgagagcatgtccagcatcaggagcaagtgccccgtggaggaaagcgaggccagcacaccacccagcaccctgccctgcctgtgcatccacgctatgacacccgagagggtgcagcggctgaaggccagcaggcccgagcagatcaccgtgtgcagctccttcccactgcccaagtacaggatcaccggcgtgcagaagatccagtgcagccagcccatcctgttcagcccaaaggtgcccgcctacatccaccccaggaagtacctggtggagaccccacccgtggacgagacacccgagccaagcgccgagaaccagagcaccgagggcacacccgagcagccacccctgatcaccgaggacgagacaaggacccggaccccagagcccatcattatcgaggaagaggaagaggacagcatcagcctgctgagcgacggccccacccaccaggtgctgcaggtggaggccgacatccacggcccacccagcgtgtccagctccagctggagcatcccacacgccagcgacttcgacgtggacagcctgagcatcctggacaccctggagggcgccagcgtgacctccggcgccaccagcgccgagaccaacagctacttcgccaagagcatggagttcctggccaggcccgtgccagctcccaggaccgtgttcaggaacccaccccacccagctcccaggaccaggaccccaagcctggctcccagcagggcctgcagcaggaccagcctggtgagcaccccacccggcgtgaacagggtgatcaccagggaggaactggaggccctgacacccagcaggacccccagcaggtccgtgagcaggactagtctggtgtccaacccacccggcgtgaacagggtgatcaccagggaggaattcgaggccttcgtggcccagcaacagagacggttcgacgccggcgcctacatcttcagcagcgacaccggccagggacacctgcagcaaaagagcgtgaggcagaccgtgctgagcgaggtggtgctggagaggaccgagctggaaatcagctacgcccccaggctggaccaggagaaggaggaactgctcaggaagaaactgcagctgaaccccaccccagccaacaggagcaggtaccagagcaggaaggtggagaacatgaaggccatcaccgccaggcggatcctgcagggcctgggacactacctgaaggccgagggcaaggtggagtgctacaggaccctgcaccccgtgccactgtacagctccagcgtgaacagggccttctccagccccaaggtggccgtggaggcctgcaacgctatgctgaaggagaacttccccaccgtggccagctactgcatcatccccgagtacgacgcctacctggacatggtggacggcgccagctgctgcctggacaccgccagcttctgccccgccaagctgaggagcttccccaagaaacacagctacctggagcccaccatcaggagcgccgtgcccagcgccatccagaacaccctgcagaacgtgctggccgctgccaccaagaggaactgcaacgtgacccagatgagggagctgcccgtgctggacagcgctgccttcaacgtggagtgcttcaagaaatacgcctgcaacaacgagtactgggagaccttcaaggagaaccccatcaggctgaccgaagagaacgtggtgaactacatcaccaagctgaagggccccaaggccgctgccctgttcgctaagacccacaacctgaacatgctgcaggacatcccaatggacaggttcgtgatggacctgaagagggacgtgaaggtgacacccggcaccaagcacaccgaggagaggcccaaggtgcaggtgatccaggccgctgacccactggccaccgcctacctgtgcggcatccacagggagctggtgaggcggctgaacgccgtgctgctgcccaacatccacaccctgttcgacatgagcgccgaggacttcgacgccatcatcgccgagcacttccagcccggcgactgcgtgctggagaccgacatcgccagcttcgacaagagcgaggatgacgctatggccctgaccgctctgatgatcctggaggacctgggcgtggacgccgagctgctcaccctgatcgaggctgccttcggcgagatcagctccatccacctgcccaccaagaccaagttcaagttcggcgctatgatgaaaagcggaatgttcctgaccctgttcgtgaacaccgtgatcaacattgtgatcgccagcagggtgctgcgggagaggctgaccggcagcccctgcgctgccttcatcggcgacgacaacatcgtgaagggcgtgaaaagcgacaagctgatggccgacaggtgcgccacctggctgaacatggaggtgaagatcatcgacgccgtggtgggcgagaaggccccctacttctgcggcggattcatcctgtgcgacagcgtgaccggcaccgcctgcagggtggccgaccccctgaagaggctgttcaagctgggcaagccactggccgctgacgatgagcacgacgatgacaggcggagggccctgcacgaggaaagcaccaggtggaacagggtgggcatcctgagcgagctgtgcaaggccgtggagagcaggtacgagaccgtgggcaccagcatcatcgtgatggctatgaccacactggccagctccgtcaagagcttctcctacctgaggggggcccctataactctctacggctaacctgaatggactacgacatagtctagtccgccaaggccgccaccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgacagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagtccctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaactcgagccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatgagcaagatctacatcgacgagcggagcaacgccgagatcgtgtgcgaggccatcaagaccatcggcatcgagggcgccaccgccgcccagctgaccaggcagctgaacatggagaagcgggaggtgaacaaggccctgtacgacctgcagaggagcgctatggtgtactccagcgacgacatccctccccggtggttcatgaccaccgaggccgacaagcccgacgccgacgctatggccgacgtgatcatcgacgacgtgagcagggagaagtccatgagggaggaccacaagagcttcgacgacgtgatccccgccaagaagatcatcgactggaagggcgccaaccccgtgaccgtgatcaacgagtactgccagatcaccaggagggactggagcttccggatcgagagcgtgggccccagcaacagccccaccttctacgcctgcgtggacatcgacggcagggtgttcgacaaggccgacggcaagagcaagcgggacgccaagaacaacgccgccaagctggccgtggacaagctgctgggctacgtgatcatccggttctaaacaattggcaagctgcttacatagaactcgcggcgattggcatgccgccttaaaatttttattttattttttcttttcttttccgaatcggattttgtttttaatatttcaaaaaaaaaaaaaaaaaaaaaaaaatctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa